SEQUENCE LISTING (199 sequences)

SEQ ID NO: 1 - protein, 6 aa, Artificial Sequence
  Synthesized
XXXXRF

SEQ ID NO: 2 - protein, 6 aa, Artificial Sequence
  Synthesized
XXXXFH

SEQ ID NO: 3 - protein, 6 aa, Artificial Sequence
  Synthesized
XXXXKF

SEQ ID NO: 4 - protein, 6 aa, Artificial Sequence
  Synthesized
XXXXQR

SEQ ID NO: 5 - protein, 6 aa, Artificial Sequence
  Synthesized
XXXXRM

SEQ ID NO: 6 - protein, 6 aa, Artificial Sequence
  Synthesized
XXXXHM

SEQ ID NO: 7 - protein, 6 aa, Artificial Sequence
  Synthesized
XXXXKL

SEQ ID NO: 8 - protein, 6 aa, Artificial Sequence
  Synthesized
XXXXRL

SEQ ID NO: 9 - protein, 6 aa, Artificial Sequence
  Synthesized
XXXLRF

SEQ ID NO: 10 - protein, 6 aa, Artificial Sequence
  Synthesized
XXXIRF

SEQ ID NO: 11 - protein, 6 aa, Artificial Sequence
  Synthesized
XXXFRF

SEQ ID NO: 12 - protein, 6 aa, Artificial Sequence
  Synthesized
XXXWRF

SEQ ID NO: 13 - protein, 6 aa, Artificial Sequence
  Synthesized
XXXMRF

SEQ ID NO: 14 - protein, 6 aa, Artificial Sequence
  Synthesized
XXKLRF

SEQ ID NO: 15 - protein, 6 aa, Artificial Sequence
  Synthesized
XXRLRF

SEQ ID NO: 16 - protein, 6 aa, Artificial Sequence
  Synthesized
XXHLRF

SEQ ID NO: 17 - protein, 6 aa, Artificial Sequence
  Synthesized
XXTLRF

SEQ ID NO: 18 - protein, 6 aa, Artificial Sequence
  Synthesized
XXFLRF

SEQ ID NO: 19 - protein, 6 aa, Artificial Sequence
  Synthesized
XXSLRF

SEQ ID NO: 20 - protein, 6 aa, Artificial Sequence
  Synthesized
XXILRF

SEQ ID NO: 21 - protein, 6 aa, Artificial Sequence
  Synthesized
XXLLRF

SEQ ID NO: 22 - protein, 6 aa, Artificial Sequence
  Synthesized
XXALRF

SEQ ID NO: 23 - protein, 6 aa, Artificial Sequence
  Synthesized
XXWLRF

SEQ ID NO: 24 - protein, 6 aa, Artificial Sequence
  Synthesized
XXMLRF

SEQ ID NO: 25 - protein, 3 aa, Artificial Sequence
  Synthesized
FRF

SEQ ID NO: 26 - protein, 3 aa, Artificial Sequence
  Synthesized
LRF

SEQ ID NO: 27 - protein, 3 aa, Artificial Sequence
  Synthesized
WRF

SEQ ID NO: 28 - protein, 3 aa, Artificial Sequence
  Synthesized
HRF

SEQ ID NO: 29 - protein, 4 aa, Artificial Sequence
  Synthesized
FLRF

SEQ ID NO: 30 - protein, 4 aa, Artificial Sequence
  Synthesized
WLRF

SEQ ID NO: 31 - protein, 5 aa, Artificial Sequence
  Synthesized
FHLRF

SEQ ID NO: 32 - protein, 6 aa, Artificial Sequence
  Synthesized
FFKLRF

SEQ ID NO: 33 - protein, 6 aa, Artificial Sequence
  Synthesized
VFKLRF

SEQ ID NO: 34 - protein, 6 aa, Artificial Sequence
  Synthesized
HFKLRF

SEQ ID NO: 35 - protein, 6 aa, Artificial Sequence
  Synthesized
IFKLRF

SEQ ID NO: 36 - protein, 6 aa, Artificial Sequence
  Synthesized
KRKLRF

SEQ ID NO: 37 - protein, 6 aa, Artificial Sequence
  Synthesized
LFKLRF

SEQ ID NO: 38 - protein, 6 aa, Artificial Sequence
  Synthesized
YFKLRF

SEQ ID NO: 39 - protein, 7 aa, Artificial Sequence
  Synthesized
FHFKLRF

SEQ ID NO: 40 - protein, 7 aa, Artificial Sequence
  Synthesized
IHFKLRF

SEQ ID NO: 41 - protein, 6 aa, Artificial Sequence
  Synthesized
FRLKFH

SEQ ID NO: 42 - protein, 6 aa, Artificial Sequence
  Synthesized
RFKLRF

SEQ ID NO: 43 - protein, 6 aa, Artificial Sequence
  Synthesized
SFKLRF

SEQ ID NO: 44 - protein, 6 aa, Artificial Sequence
  Synthesized
MFKLRF

SEQ ID NO: 45 - protein, 6 aa, Artificial Sequence
  Synthesized
TFKLRF

SEQ ID NO: 46 - protein, 6 aa, Artificial Sequence
  Synthesized
QFKLRF

SEQ ID NO: 47 - protein, 6 aa, Artificial Sequence
  Synthesized
WFKLRF

SEQ ID NO: 48 - protein, 44 aa, Tachypleus tridentatus
  misc_feature: Tachystatin A Peptide
YSRCQLQGFNCVVRSYGLPTIPCCRGLTCRSYFPGSTYGRCQRY

SEQ ID NO: 49 - protein, 25 aa, Androctonus australis
RSVCRQIKICRRRGGCYYKCTNRPY

SEQ ID NO: 50 - protein, 13 aa, Unknown
  Synthetic Tritrpticin
VRRFPWWWPFLRR

SEQ ID NO: 51 - protein, 30 aa, Homo sapiens
  misc_feature: HNP-3 Defensin
DCYCRIPACIAGERRYGTCIYQGRLWAFCC

SEQ ID NO: 52 - protein, 38 aa, Phytolacca americana
AGCIKNGGRCNASAGPPYCCSSYCFQIAGQSYGVCKNR

SEQ ID NO: 53 - protein, 23 aa, Unknown
  Synthetic construct Magainin 2
GIGKYLHSAKKFGKAWVGEIMNS

SEQ ID NO: 54 - protein, 13 aa, Bos taurus
ILPWKWPWWPWRR

SEQ ID NO: 55 - protein, 44 aa, Heliothis virescens
DKLIGSCVWGAVNYTSDCNGECKRRGYKGGHCGSFANVNCWCET

SEQ ID NO: 56 - protein, 44 aa, Heliothis virescens; identical to SEQ ID NO: 55

SEQ ID NO: 57 - protein, 46 aa, Pisum sativum
  misc_feature: Seed of pea defensin 1 (psd1)
KTCEHLADTYRGVCFTNASCDDHCKNKAHLISGTCHNWKCFCTQNC

SEQ ID NO: 58 - protein, 18 aa, Unknown
  Synthetic Gomesin
QCRRLCYKQRCVTYCRGR

SEQ ID NO: 59 - protein, 25 aa, Bos taurus
  misc_feature: Lactoferricin B
FKCRRWQWRMKKLGAPSITCVRRAF

SEQ ID NO: 60 - protein, 12 aa, Unknown
  Synthetic PW2
HPLKQYWWRPSI

SEQ ID NO: 61 - protein, 20 aa, Homo sapiens
  misc_feature: Hepcidin 20
ICIFCCGCCHRSKCGMCCKT

SEQ ID NO: 62 - protein, 25 aa, Homo sapiens
  misc_feature: Hepcidin 25
DTHFPICIFCCGCCHRSKCGMCCKT

SEQ ID NO: 63 - protein, 30 aa, Amaranthus caudatus
VGECVRGRCPSGMCCSQFGYCGKGPKYCGR

SEQ ID NO: 64 - protein, 78 aa, Amaranthus caudatus
GYFCESCRKIIQKLEDMVGPQPNEDTVTQAASQVCDKLKILRGLCKKIMRSFLRRISWDILTGKKPQAICVDIKICKE

SEQ ID NO: 65 - protein, 23 aa, Xenopus laevis
  misc_feature: Magainin 2
GIGKFLHSAKKFGKAFVGEIMNS

SEQ ID NO: 66 - protein, 26 aa, Apis mellifera
  misc_feature: venom Melittin B
GIGAVLKVLTTGLPALISWIKRKRQQ

SEQ ID NO: 67 - protein, 21 aa, Podisus maculiventris
GSKKPVPIIYCNRRTGKCQRM

SEQ ID NO: 68 - protein, 38 aa, Mesembryanthemum crystallinum
  misc_feature: Antimicrobial peptide 1
AKCIKNGKGCREDQGPPFCCSGFCYRQVGWARGYCKNR

SEQ ID NO: 69 - protein, 13 aa, Bos taurus
  misc_feature: Melanotropin alpha (Alpha-MSH)
SYSMEHFRWGKPV

SEQ ID NO: 70 - protein, 33 aa, Oryctolagus cuniculus
  misc_feature: Corticostatin III (MCP-1)
VVCACRRALCLPRERRAGFCRIRGRIHPLCCRR

SEQ ID NO: 71 - protein, 33 aa, Oryctolagus cuniculus
  misc_feature: Corticostatin IV (MCP-2)
VVCACRRALCLPLERRAGFCRIRGRIHPLCCRR

SEQ ID NO: 72 - protein, 35 aa, Antheraea pernyi
  misc_feature: Cecropin B
KWKIFKKIEKVGRNIRNGIIKAGPAVAVLGEAKAL

SEQ ID NO: 73 - protein, 48 aa, Bos taurus
  misc_feature: Seminalplasmin
SDEKASPDKHHRFSLSRYAKLANRLANPKLLETFLSKWIGDRGNRSVK

SEQ ID NO: 74 - protein, 34 aa, Oryctolagus cuniculus
  misc_feature: NP-3A defensin
GICACRRRFCPNSERFSGYCRVNGARYVRCCSRR

SEQ ID NO: 75 - protein, 30 aa, Homo sapiens
  misc_feature: HNP-1 Defensin
ACYCRIPACIAGERRYGTCIYQGRLWAFCC

SEQ ID NO: 76 - protein, 29 aa, Homo sapiens
  misc_feature: HNP-2 Defensin
CYCRIPACIAGERRYGTCIYQGRLWAFCC

SEQ ID NO: 77 - protein, 33 aa, Homo sapiens
  misc_feature: HNP-4 Defensin
VCSCRLVFCRRTELRVGNCLIGGVSFTYCCTRV

SEQ ID NO: 78 - protein, 24 aa, Homo sapiens
  misc_feature: Histatin 5
DSHAKRHHGYKRKFHEKHHSHRGY

SEQ ID NO: 79 - protein, 32 aa, Homo sapiens
  misc_feature: Histatin 3
DSHAKRHHGYKRKFHEKHHSHRGYRSNYLYDN

SEQ ID NO: 80 - protein, 12 aa, Unknown
  Histatin 8
KFHEKHHSHRGY

SEQ ID NO: 81 - protein, 38 aa, Bos taurus
  misc_feature: Tracheal antimicrobial peptide
NPVSCVRNKGICVPIRCPGSMKQIGTCVGRAVKCCRKK

SEQ ID NO: 82 - protein, 37 aa, Mirabilis jalapa
  misc_feature: antimicrobial peptidic agent1 (MJ-antimicrobial peptidic agent1)
QCIGNGGRCNENVGPPYCCSGFCLRQPGQGYGYCKNR

SEQ ID NO: 83 - protein, 36 aa, Mirabilis jalapa
  misc_feature: antimicrobial peptidic agent2 (MJ-antimicrobial peptidic agent2)
CIGNGGRCNENVGPPYCCSGFCLRQPNQGYGVCRNR

SEQ ID NO: 84 - protein, 33 aa, Zea mays
  misc_feature: MBP-1
RSGRGECRRQCLRRHEGQPWETQECMRRCRRRG

SEQ ID NO: 85 - protein, 23 aa, Brassica napus
  misc_feature: AFP2
QKLCERPSGTWSGVCGNNNACKN

SEQ ID NO: 86 - protein, 27 aa, Brassica rapa
  misc_feature: AFP1
QKLCERPSGTWSGVCGNNNACKNQCIN

SEQ ID NO: 87 - protein, 27 aa, Brassica rapa
  misc_feature: AFP2
QKLCERPSGTWSGVCGNNNACKNQCIR

SEQ ID NO: 88 - protein, 33 aa, Phyllomedusa bicolor
  misc_feature: Adenoregulin
GLWSKIKEVGKEAAKAAAKAAGKAALGAVSEAV

SEQ ID NO: 89 - protein, 16 aa, Sus scrofa
  misc_feature: Protegrin 2
RGGRLCYCRRRFCICV

SEQ ID NO: 90 - protein, 18 aa, Sus scrofa
  misc_feature: Protegrin 3
RGGGLCYCRRRFCVCVGR

SEQ ID NO: 91 - protein, 38 aa, Macaca fascicularis
  misc_feature: Histatin 1
DSHEERHHGRHGHHKYGRKFHEKHHSHRGYRSNYLYDN

SEQ ID NO: 92 - protein, 24 aa, Xenopus laevis
  misc_feature: Peptide PGQ
GVLSNVIGYLKKLGTGALNAVLKQ

SEQ ID NO: 93 - protein, 20 aa, Rana catesbeiana
  misc_feature: Ranalexin
FLGGLIKIVPAMICAVTKKC

SEQ ID NO: 94 - protein, 30 aa, Cavia cutleri
  misc_feature: GNCP-2
RCICTTRTCRFPYRRLGTCLFQNRVYTFCC

SEQ ID NO: 95 - protein, 18 aa, Sus scrofa
  misc_feature: Protegrin 4
RGGRLCYCRGWICFCVGR

SEQ ID NO: 96 - protein, 18 aa, Sus scrofa
  misc_feature: Protegrin 5
RGGRLCYCRPRFCVCVGR

SEQ ID NO: 97 - protein, 27 aa, Bos taurus
  misc_feature: BMAP-27
GRFKRFRKKFKKLFKKLSPVIPLLHLG

SEQ ID NO: 98 - protein, 28 aa, Bos taurus
  misc_feature: BMAP-28
GGLRSLGRKILRAWKKYGPIIVPIIRIG

SEQ ID NO: 99 - protein, 39 aa, Bufo bufo gargarizans
  misc_feature: Buforin 1
AGRGKQGGKVRAKAKTRSSRAGLQFPVGRVHRLLRKGNY

SEQ ID NO: 100 - protein, 21 aa, Bufo bufo gargarizans
  misc_feature: Buforin II
TRSSRAGLQFPVGRVHRLLRK

SEQ ID NO: 101 - protein, 34 aa, Bos taurus
  misc_feature: BMAP-34
GLFRRLRDSIRRGQQKILEKARRIGERIKDIFRG

SEQ ID NO: 102 - protein, 19 aa, Trichoderma longibrachiatum
  misc_feature: Tricholongin
AGFAAQAAASLAPVAAQQL

SEQ ID NO: 103 - protein, 34 aa, Phyllomedusa sauvagei
  misc_feature: Dermaseptin 1
ALWKTMLKKLGTMALHAGKAALGAAADTISQGTQ

SEQ ID NO: 104 - protein, 45 aa, Hevea brasiliensis
  misc_feature: Pseudo-hevein (Minor hevein)
EQCGRQAGGKLCPNNLCCSQYGWCGSSDDYCSPSKNCQSNCKGGG

SEQ ID NO: 105 - protein, 33 aa, Rana rugosa
  misc_feature: Gaegurin-1
SLFSLIKAGAKFLGKNLLKQGACYAACKASKQC

SEQ ID NO: 106 - protein, 36 aa, Phyllomedusa bicolor
  misc_feature: Skin peptide tyrosine-tyrosine
YPPKPESPGEDASPEEMNKYLTALRHYINLVTRQRY

SEQ ID NO: 107 - protein, 50 aa, Penaeus vannamei
  misc_feature: Penaeidin-1
YRGGYTGPIPRPPPIGRPPLRLVVCACYRLSVSDARNCCIKFGSCCHLVK

SEQ ID NO: 108 - protein, 33 aa, Mesocricetus auratus
  misc_feature: Neutrophil defensin 1 (HANP-1)
VTCFCRRRGCASRERHIGYCRFGNTIYRLCCRR

SEQ ID NO: 109 - protein, 33 aa, Mesocricetus auratus
  misc_feature: Neutrophil defensin 3 (HANP-3)
VTCFCRRRGCASRERLIGYCRFGNTIYGLCCRR

SEQ ID NO: 110 - protein, 21 aa, Misgurnus anguillicaudatus
  misc_feature: Misgurin
RQRVEELSKFSKKGAAARRRK

SEQ ID NO: 111 - protein, 41 aa, Pharbitis nil
  misc_feature: PN-antimicrobial peptidic agent
QQCGRQASGRLCGNRLCCSQWGYCGSTASYCGAGCQSQCRS

SEQ ID NO: 112 - protein, 19 aa, Oncorhynchus mykiss
  misc_feature: Histone H2B-1(HLP-1)(Fragment)
PDPAKTAPKKGSKKAVTKA

SEQ ID NO: 113 - protein, 17 aa, Oncorhynchus mykiss
  misc_feature: Histone H2B-3(HLP-3)(Fragment)
PDPAKTAPKKKSKKAVT

SEQ ID NO: 114 - protein, 30 aa, Macaca mulatta
  misc_feature: neutrophil defensin 2 (RMAD-2)
ACYCRIPACLAGERRYGTCFYMGRVWAFCC

SEQ ID NO: 115 - protein, 36 aa, Pseudacanthotermes spiniger
  misc_feature: Termicin
ACNFQSCWATCQAQHSIYFRRAFCDRSQCKCVFVRG

SEQ ID NO: 116 - protein, 25 aa, Pseudacanthotermas spiniger
  misc_feature: Spingerin
HVDKKVADKVLLLKQLRIMRLLTRL

SEQ ID NO: 117 - protein, 13 aa, Litoria raniformis
  misc_feature: Aurein 1.1
GLFDIIKKIAESI

SEQ ID NO: 118 - protein, 30 aa, Pachycondyla goeldii
  misc_feature: Ponericin G1
GWKDWAKKAGGWLKKKGPGMAKAALKAAMQ

SEQ ID NO: 119 - protein, 24 aa, Rana berlandieri
  misc_feature: Brevinin-1BB
FLPAIAGMAAKFLPKIFCAISKKC

SEQ ID NO: 120 - protein, 20 aa, Rana clamitans
  misc_feature: Ranalexin-1CB
FLGGLMKAFPAIICAVTKKC

SEQ ID NO: 121 - protein, 31 aa, Rana clamitans
  misc_feature: Ranatuerin-2CA
GLFLDTLKGAAKDVAGKLLEGLKCKIAGCKP

SEQ ID NO: 122 - protein, 27 aa, Rana clamitans
  misc_feature: Ranatuerin-2CB
GLFLDTLKGLAGKLLQGLKCIKAGCKP

SEQ ID NO: 123 - protein, 40 aa, Ginkgo biloba
  misc_feature: Ginkbilobin
ANTAFVSSAHNTQKIPAGAPFNRNLRAMLADLRQNAAFAG

SEQ ID NO: 124 - protein, 20 aa, Basella alba
  misc_feature: Alpha-basrubrin (Fragment)
GADFQECMKEHSQKQHQHQG

SEQ ID NO: 125 - protein, 24 aa, Pseudis paradoxa
  misc_feature: Pseudin 1
GLNTLKKVFQGLHEAIKLINNHVQ

SEQ ID NO: 126 - protein, 45 aa, Parabuthus schlechteri
  misc_feature: Parabutoporin
FKLGSFLKKAWKSKLAKKLRAKGKEMLKDYAKGLLEGGSEEVPGQ

SEQ ID NO: 127 - protein, 44 aa, Opistophthalmus carinatus
  misc_feature: Opistoporin 1
GKVWDWIKSTAKKLWNSEPVKELKNTALNAAKNLVAEKIGATPS

SEQ ID NO: 128 - protein, 44 aa, Opistophthalmus carinatus
  misc_feature: Opistoporin 2
GKVWDWIKSTAKKLWNSEPVKELKNTALNAAKNFVAEKIGATPS

SEQ ID NO: 129 - protein, 12 aa, Oncorhynchus mykiss
  misc_feature: Histone H2A (Fragment)
AERVGAGAPVYL

SEQ ID NO: 130 - protein, 33 aa, Dolabella auricularia
  misc_feature: Dolabellanin B2
SHQDCYEALHKCMASHSKPFSCSMKFHMCLQQQ

SEQ ID NO: 131 - protein, 35 aa, Heliothis virescens
  misc_feature: Cecropin A
RWKVFKKIEKVGRNIRDGVIKAAPAIEVLGQAKAL

SEQ ID NO: 132 - protein, 35 aa, Homo sapiens
  misc_feature: HNP-5 Defensin
QARATCYCRTGRCATRESLSGVCEISGRLYRLCCR

SEQ ID NO: 133 - protein, 35 aa, Homo sapiens
  misc_feature: HNP-6 Defensin
STRAFTCHCRRSCYSTEYSYGTCTVMGINHRFCCL

SEQ ID NO: 134 - protein, 84 aa, Holotrichia diomphalia
  misc_feature: Holotricin 3
YGPGDGHGGGHGGGHGGGHGNGQGGGHGHGPGGGFGGGHGGGHGGGGRGGGGSGGGGSPGHGAGGGYPGGHGGGHHGGYQTHGY

SEQ ID NO: 135 - protein, 45 aa, Bos taurus
  misc_feature: Lingual antimicrobial peptide
GFTQGVRNSQSCRRNKGICVPIRCPGSMRQIGTCLGAQVKCCRRK

SEQ ID NO: 136 - protein, 29 aa, Rattus norvegicus
  misc_feature: RatNP-3
CSCRTSSCRFGERLSGACRLNGRIYRLCC

SEQ ID NO: 137 - protein, 31 aa, Cavia cutleri
  misc_feature: GNCP-1
RRCICTTRTCRFPYRRLGTCIFQNRVYTFCC

SEQ ID NO: 138 - protein, 47 aa, Penaeus vannamei
  misc_feature: Penaeidin-4a
HSSGYTRPLPKPSRPIFIRPIGCDVCYGIPSSTARLCCFRYGDCCHR

SEQ ID NO: 139 - protein, 6 aa, Bos taurus
  misc_feature: hexapeptide
RRWQWR

SEQ ID NO: 140 - protein, 18 aa, Penaeus vannamei
KWKLFKKIPKFLHLAKKF

SEQ ID NO: 141 - protein, 20 aa, Homo sapiens
  misc_feature: MUC7 20-Mer
LAHQKPFIRKSYKCLHKRCR

SEQ ID NO: 142 - protein, 21 aa, Rana nigromaculata
  misc_feature: Nigrocin 2
GLLSKVLGVGKKVLCGVSGLC

SEQ ID NO: 143 - protein, 33 aa, Rana nigromaculata
  misc_feature: Nigrocin 1
GLLDSIKGMAISAGKGALQNLLKVASCKLDKTC

SEQ ID NO: 144 - protein, 10 aa, Unknown
  lactoferrin (Lf) peptide 2
FKCRRWQWRM

SEQ ID NO: 145 - protein, 18 aa, Impatiens balsamina
  misc_feature: Ib-antimicrobial peptidic agent3
RHRCCAWGPGRKYCKRWC

SEQ ID NO: 146 - protein, 18 aa, Impatiens balsamina
  misc_feature: Ib-antimicrobial peptidic agent4
GRRCCGWGPGRRYCRRWC

SEQ ID NO: 147 - protein, 14 aa, Unknown
  Synthesis dhvar4
KRLFKKLLFSLRKY

SEQ ID NO: 148 - protein, 14 aa, Unknown
  Synthesis dhvar5
LLLFLLKKRKKRKY

SEQ ID NO: 149 - protein, 6 aa, Artificial Sequence
  Synthesized
XXXXXC

SEQ ID NO: 150 - protein, 6 aa, Artificial Sequence
  Synthesized
XXXXFC

SEQ ID NO: 151 - protein, 6 aa, Artificial Sequence
  Synthesized
XXXXNC

SEQ ID NO: 152 - protein, 6 aa, Artificial Sequence
  Synthesized
XXXXWC

SEQ ID NO: 153 - protein, 6 aa, Artificial Sequence
  Synthesized
XXXXIC

SEQ ID NO: 154 - protein, 6 aa, Artificial Sequence
  Synthesized
XXXXTC

SEQ ID NO: 155 - protein, 6 aa, Artificial Sequence
  Synthesized
XXXXYC

SEQ ID NO: 156 - protein, 6 aa, Artificial Sequence
  Synthesized
XXXXVC

SEQ ID NO: 157 - protein, 6 aa, Artificial Sequence
  Synthesized
XXXXMC

SEQ ID NO: 158 - protein, 6 aa, Artificial Sequence
  Synthesized
XXXXGC

SEQ ID NO: 159 - protein, 6 aa, Artificial Sequence
  Synthesized
XXXXEC

SEQ ID NO: 160 - protein, 6 aa, Artificial Sequence
  Synthesized
XXXXSC

SEQ ID NO: 161 - protein, 6 aa, Artificial Sequence
  Synthesized
XXXXLC

SEQ ID NO: 162 - protein, 6 aa, Artificial Sequence
  Synthesized
XXXXPC

SEQ ID NO: 163 - protein, 6 aa, Artificial Sequence
  Synthesized
XXXXHC

SEQ ID NO: 164 - protein, 6 aa, Artificial Sequence
  Synthesized
XXXXCC

SEQ ID NO: 165 - protein, 6 aa, Artificial Sequence
  Synthesized
XXXXAC

SEQ ID NO: 166 - protein, 6 aa, Artificial Sequence
  Synthesized
XXXXKC

SEQ ID NO: 167 - protein, 6 aa, Artificial Sequence
  Synthesized
XXXXQC

SEQ ID NO: 168 - protein, 6 aa, Artificial Sequence
  Synthesized
XXXXRC

SEQ ID NO: 169 - protein, 6 aa, Artificial Sequence
  Synthesized
XXXXDC

SEQ ID NO: 170 - protein, 6 aa, Artificial Sequence
  Synthesized
XXXXCY

SEQ ID NO: 171 - protein, 6 aa, Artificial Sequence
  Synthesized
XXXXCQ

SEQ ID NO: 172 - protein, 6 aa, Artificial Sequence
  Synthesized
XXXXYS

SEQ ID NO: 173 - protein, 6 aa, Artificial Sequence
  Synthesized
XXXXYD

SEQ ID NO: 174 - protein, 6 aa, Artificial Sequence
  Synthesized
XXXXTQ

SEQ ID NO: 175 - protein, 6 aa, Artificial Sequence
  Synthesized
WTFRYC

SEQ ID NO: 176 - protein, 6 aa, Artificial Sequence
  Synthesized
CYRFTW

SEQ ID NO: 177 - protein, 11 aa, Glomerella cingulata
GYFSYPHGNLF

SEQ ID NO: 178 - protein, 13 aa, Saccharomyces cerevisiae
WHWLQLKPGQPMY

SEQ ID NO: 179 - protein, 13 aa, Saccharomyces kluyveri
WHWLSFSKGQPMY

SEQ ID NO: 180 - protein, 19 aa, Artificial Sequence
  Synthesized
YNLEDHPQGDHPKLQLWHW

SEQ ID NO: 181 - protein, 15 aa, Artificial Sequence
  Synthesized
YNLEPQGPKLQLWHW

SEQ ID NO: 182 - protein, 13 aa, Artificial Sequence
  Synthesized
YMPQGPKLQLFHW

SEQ ID NO: 183 - protein, 12 aa, Artificial Sequence
  Synthesized
YMPQGPKLQLWH

SEQ ID NO: 184 - protein, 13 aa, Artificial Sequence
  Synthesized
YMPQGPRLNLWHW

SEQ ID NO: 185 - protein, 26 aa, Magnaporthe grisea
MSPSTKNIPAPVAGARAGPIHYCVIM

SEQ ID NO: 186 - protein, 24 aa, Neurospora crassa
MPSTAASTRVPQTTMNFNGYCVVM

SEQ ID NO: 187 - protein, 23 aa, Cryphonectria parasitica
MPSNTQTSNSSMGVNGYSYCVVM

SEQ ID NO: 188 - protein, 11 aa, Magnaporthe grisea
QWCPRRGQPCW

SEQ ID NO: 189 - protein, 11 aa, Neurospora crassa
QWCRIHGQSCW

SEQ ID NO: 190 - protein, 13 aa, Saccharomyces cerevisiae
WHWLQLKPGQPMY

SEQ ID NO: 191 - protein, 10 aa, Cryphonectria parasitica
WCLFHGEGCW

SEQ ID NO: 192 - protein, 4 aa, Artificial Sequence
  Synthesized
XAAC

SEQ ID NO: 193 - protein, 10 aa, Artificial Sequence
  Synthesized
WCXXGXXXCW

SEQ ID NO: 194 - protein, 6 aa, Artificial Sequence
  Synthesized
XXXXCI

SEQ ID NO: 195 - protein, 10 aa, Fusarium graminearum
WCQQKGQPCW

SEQ ID NO: 196 - protein, 10 aa, Fusarium graminearum
WCTWKGQPCW

SEQ ID NO: 197 - protein, 7 aa, Artificial Sequence
  Synthesized
FRLKFHF

SEQ ID NO: 198 - protein, 6 aa, Artificial Sequence
  Synthesized
FRLKHI

SEQ ID NO: 199 - protein, 5 aa, Artificial Sequence
  Synthesized
FRLHF

(19) United States
(12) Patent Application Publication
(10) Pub. No.: US 2009/0238811 A1
(43) Pub. Date: Sep. 24, 2009

(54) Enzymatic Antimicrobial and Antifouling Coatings and Polymeric Materials

(76) Inventors: C. Steven McDaniel, Austin, TX (US); Melinda E. Wales, Bryan, TX (US); Juan Carlo Carvajal, Austin, TX (US)

Correspondence Address: C. Steven McDaniel;c/o Daffer McDaniel, LLP, P.O. Box 684908, Austin, TX, 78768-4908, US

(21) Appl. No.: 12/243,755

(22) Filed: Oct. 1, 2008

Related U.S. Application Data
(63) Continuation-in-part of application No. 10/655,345, filed on Sep. 4, 2003.
(60) Provisional application No. 60/976,676, filed on Oct. 1, 2007.
(60) Provisional application No. 60/409,102, filed on Sep. 9, 2002.

(51) Int. Cl. A61K 38/54; C12N 9/14; C12N 9/48

(52) U.S. Cl. 424/94.2; 435/195; 435/212

(57) ABSTRACT

Disclosed herein are a coating, a textile finish, a wax, elastomer, a filler, an adhesive, or a sealant, as well as polymeric materials such as a plastic, a laminate, a composite, that includes an enzyme that degrades cell wall or cell membrane components (e.g., a lysozyme, lytic transgrycosylase) alone or in combination with other enzymes such as a lipolytic enzyme, a sulfuric ester hydrolase, an organophosphorus compound degradation enzyme, or an antimicrobial peptide. Also disclosed herein are methods of retarding or preventing microbial growth on or in a coating, paint, textile finish, wax, elastomer, adhesive, sealant, filler, or a polymeric material, where such a surface material includes an enzyme that degrades cell wall or cell membrane components (e.g., a lysozyme, lytic transgrycosylase).

### PRIORITY CLAIM

This application claims priority to U.S. Provisional Application No. 60/976,676 filed Oct. 1, 2007 and is a Continuation-in-Part of U.S. application Ser. No. 10/655,345 filed Sep. 4, 2003, which claims priority to U.S. Provisional Application No. 60/409,102 filed Sep. 9, 2002.

### BACKGROUND OF THE INVENTION

A. Field of the Invention

The invention relates generally to surface treatments such as a coating (e.g., a paint, a clear coat), a textile finish, a wax, elastomer, a filler, an adhesive, or a sealant, as well as polymeric materials such as a plastic, a laminate, a composite, comprising an enzyme that degrades cell wall or cell membrane components (e.g., a lysozyme, lytic transgrycosylase) alone or in combination with other enzymes (e.g., lipolytic enzyme, sulfuric ester hydrolase, organophosphorus compound degradation enzyme) that may confer additional properties to a coating or polymeric material.

B. Description of the Related Art

A coating (e.g., a paint, a clear coat), a textile finish, a wax, an elastomer, an adhesive, a sealant, or a filler generally are compositions used, for example, to protect, decorate, attach, or seal one or more surfaces, and underlying material(s), that they are applied. Such materials are commonly used in commercial and industrial applications. For example, a coating such as paint typically forms a solid protective, decorative, or functional adherent film on a surface. Polymeric materials comprise molecular polymers to form various shaped materials typically for consumer or industrial products, and whose surface of the polymeric material is subject to addition of a coating.

Biomolecules are molecules often produced and isolated from organisms, such as an enzyme which catalyzes a chemical reaction. Alexander Fleming discovered lysozyme (ca. 1922) during a search for antibiotics conducted over a period of years, when he added a drop of mucus to a growing bacterial culture and discovered it killed the bacteria. Lysozymes have widespread distribution in animals and plants, where it serves as a “natural antibiotic” protecting fluids and tissues that are rich in potential food for bacterial growth, such as egg white. As a part of the innate defense mechanism, lysozyme is found in many mammalian secretions and tissues, saliva, tears, milk, cervical mucus, leucocytes, kidneys, etc.

Lipolytic enzymes catalyze a reaction on a lipid substrate, such as a vegetable oil, a phospholipid, a sterol, and other hydrophobic molecules, generally to hydrolyze or move (e.g., intraesterification) an ester bond. Often lipolytic enzyme (e.g., lipase) catalyzed reactions are used for industrial or commercial purposes, such as alcohol or acid esterification, interesterification, and transesterification reactions, acidolysis, alcoholysis, and resolution of racemic alcohol and organic acid mixtures.

Various enzymes have been identified that detoxify organophosphorus compounds (“organophosphate compounds” or “OP compounds”) compounds, such as organophosphorus hydrolase (“OPH”), organophosphorus acid anhydrolase (“OPAA”), and DFPase, which detoxifies O,O-diisopropyl phosphorofluoridate (“DFP”). Organophosphorus compounds and organosulfur (“OS”) compounds are used extensively as insecticides and are toxic to many organisms, including humans. OP compounds function as nerve agents. OP compounds have been used both as pesticides and chemical warfare agents.

Sulfuric ester hydrolase can catalyze reactions at sulfuric ester bonds. A peptidase catalyzes reactions at a peptide bond, and is reactions on peptides, polypeptides and proteins.

### SUMMARY OF THE INVENTION

In general, the invention features a coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises an antimicrobial enzyme, an antifouling enzyme, a peptidase, an esterase, a petroleum lipolytic enzyme, or a combination thereof. In specific embodiments, the antimicrobial enzyme or antifouling enzyme comprises a lysozyme, a lysostaphin, a libiase, a lysyl endopeptidase, a mutanolysin, a cellulase, a chitinase, an α-agarase, an β-agarase, an N-acetylmuramoyl-L-alanine amidase, a lytic transglycosylase, a glucan endo-1,3-β-D-glucosidase, an endo-1,3(4)-β-glucanase, a β-lytic metalloendopeptidase, a 3-deoxy-2-octulosonidase, a peptide-N4-(N-acetyl-β-glucosaminyl)asparagine amidase, a mannosyl-glycoprotein endo-β-N-acetylglucosaminidase, a ι-carrageenase, a κ-carrageenase, a λ-carrageenase, an α-neoagaro-oligosaccharide hydrolase, an endolysin, an autolysin, a mannoprotein protease, a glucanase, a mannase, a zymolase, a lyticase a lipolytic enzyme, or a combination thereof. In specific embodiments, the antimicrobial enzyme or antifouling enzyme catalyzes a reaction that degrades a cell wall or cell membrane. In specific embodiments, the lipolytic enzyme comprises a phospholipase.

In some aspects the antimicrobial enzyme or antifouling enzyme comprises a lysozyme. In some aspects the antimicrobial enzyme or antifouling enzyme comprises a lysostaphin. In some aspects the antimicrobial enzyme or antifouling enzyme comprises a libiase. In some aspects the antimicrobial enzyme or antifouling enzyme comprises a lysyl endopeptidase. In some aspects the antimicrobial enzyme or antifouling enzyme comprises a mutanolysin. In some aspects the antimicrobial enzyme or antifouling enzyme comprises a cellulase. The coating of claim 8, wherein the cellulase comprises a α-cellulase. IThe coating of claim 8, wherein the cellulase comprises a β-cellulase. In some aspects the antimicrobial enzyme or antifouling enzyme comprises a chitinase. In some aspects the antimicrobial enzyme or antifouling enzyme comprises an α-agarase. In some aspects the antimicrobial enzyme or antifouling enzyme comprises an β-agarase. In some aspects the antimicrobial enzyme or antifouling enzyme comprises an N-acetylmuramoyl-L-alanine amidase. In some aspects the antimicrobial enzyme or antifouling enzyme comprises a lytic transglycosylase. In some aspects the antimicrobial enzyme or antifouling enzyme comprises a glucan endo-1,3-β-D-glucosidase. In some aspects the antimicrobial enzyme or antifouling enzyme comprises an endo-1,3(4)-β-glucanase. In some aspects the antimicrobial enzyme or antifouling enzyme comprises a β-lytic metalloendopeptidase. In some aspects the antimicrobial enzyme or antifouling enzyme comprises a 3-deoxy-2-octulosonidase. In some aspects the antimicrobial enzyme or antifouling enzyme comprises a peptide-N4-(N-acetyl-β-glucosaminyl)asparagine amidase. In some aspects the antimicrobial enzyme or antifouling enzyme comprises a mannosyl-glycoprotein endo-β-N-acetylglucosaminidase. In some aspects the antimicrobial enzyme or antifouling enzyme comprises a ι-carrageenase. In some aspects the antimicrobial enzyme or antifouling enzyme comprises a κ-carrageenase. In some aspects the antimicrobial enzyme or antifouling enzyme comprises a λ-carrageenase. In some aspects the antimicrobial enzyme or antifouling enzyme comprises an α-neoagaro-oligosaccharide hydrolase. In some aspects the antimicrobial enzyme or antifouling enzyme comprises an endolysin. In some aspects the antimicrobial enzyme or antifouling enzyme comprises an autolysin. In some aspects the antimicrobial enzyme or antifouling enzyme comprises a mannoprotein protease. In some aspects the antimicrobial enzyme or antifouling enzyme comprises a glucanase. In some aspects the antimicrobial enzyme or antifouling enzyme comprises a mannase. In some aspects the antimicrobial enzyme or antifouling enzyme comprises a zymolase. In some aspects the antimicrobial enzyme or antifouling enzyme comprises a lyticase. In some aspects the antimicrobial enzyme or antifouling enzyme comprises a lipolytic enzyme. In some embodiments, the esterase comprises a lipolytic enzyme, a sulfuric ester hydrolase, phosphoric triester hydrolase, or a combination thereof.

In some embodiments, the peptidase possessing esterase activity.

In some embodiments, the active enzyme comprises a plurality of active enzymes.

In some embodiments, the coating comprises an interior coating.

In some embodiments, the lipolytic enzyme catalyzes a reaction on a substrate comprising a fatty acid.

In some embodiments, the lipolytic enzyme comprises a carboxylesterase, a lipase, a lipoprotein lipase, an acylglycerol lipase, a hormone-sensitive lipase, a phospholipase A₁, a phospholipases A₂, a phosphatidylinositol deacylase, a phospholipase C, a phospholipase D, a phosphoinositide phospholipase C, a phosphatidate phosphatase, a lysophospholipase, a sterol esterase, a galactolipase, a sphingomyelin phosphodiesterase, a sphingomyelin phosphodiesterases D, a ceramidase, a wax-ester hydrolase, a fatty-acyl-ethyl-ester synthase, a retinyl-palmitate esterase, a 11-cis-retinyl-palmitate hydrolase, an all-trans-retinyl-palmitate hydrolase, a cutinase, an acyloxyacyl hydrolase, or a combination thereof.

In some embodiments, the lipolytic enzyme comprises a combination of lipolytic enzymes.

In some embodiments, the lipolytic enzyme comprises a carboxylesterase derived from Actinidia deliciosa, Aedes aegypti, Aeropyrum pernix, Alicyclobacillus acidocaldarius, Aphis gossypii, Arabidopsis thaliana, Archaeoglobus fulgidus, Aspergillus clavatus, Athalia rosae, Bacillus acidocaldarius, Bombyx mandarina, Bombyx mori, Bos taurus, Burkholderia gladioli, Caenorhabditis elegans, Canis familiaris, Cavia porcellus, Chloroflexus aurantiacus, Felis catus, Fervidobacterium nodosum, Helicoverpa armigera, Homo sapiens, Macacafascicularis, Malus pumila, Mesocricetus auratus, Mus musculus, Musca domestica, Mycoplasma hyopneumoniae, Myxococcus xanthus, Neosartoryafischeri, Oryctolagus cuniculus, Paeonia suffruticosa, Pseudomonas aeruginosa, Rattus norvegicus, Rubrobacter xylanophilus, Spodoptera exigua, Spodoptera litura, Sulfolobus acidocaldarius, Sulfolobus shibatae, Sulfolobus solfataricus, Sus scrofa, Thermotoga maritime, Thermus thermophilus, Vaccinium corymbosum, Vibrio harveyi, Xenopsylla cheopis, Yarrowia lipolytica, or a combination thereof.

In some embodiments, the lipolytic enzyme comprises a thermophilic carboxylesterase derived from Aeropyrum pernix, Alicyclobacillus acidocaldarius, Archaeoglobus fulgidus, Bacillus acidocaldarius, Pseudomonas aeruginosa, Sulfolobus shibatae, Sulfolobus solfataricus, Thermotoga maritime, or a combination thereof.

In some embodiments, the lipolytic enzyme comprises a lipase derived from Acinetobacter, Aedes aegypti, Anguillajaponica, Antrodia cinnamomea, Arabidopsis rosette, Arabidopsis thaliana, Arxula adeninivorans, Aspergillus niger, Aspergillus oryzae, Aspergillus tamarii, Aureobasidium pullulans, Avena sativa, Bacillus licheniformis, Bacillus sphaericus, Bacillus stearothermophilus, Bacillus subtilis, Bacillus thermocatenulatus, Bacillus thermoleovorans, Bombyx mandarina, Bombyx mori, Bos Taurus, Brassica napus, Brassica rapa, Burkholderia cepacia, Caenorhabditis elegans, Candida albicans, Candida antarctica, Candida deformans, Candida parapsilosis, Candida rugosa, Candida thermophila, Canis domesticus, Chenopodium rubrum, Clostridium beijerinckii, Clostridium botulinum, Clostridium novyi, Danio rerio, Galactomyces geotrichum, Gallus gallus, Geobacillus, Gibberella zeae, Gossypium hirsutum, Homo sapiens, Kurtzmanomyces sp., Leishmania infantum, Lycopersicon esculentum L, Malasseziafurfur, Methanosarcina acetivorans, Mus musculus, Mus spretus, Mycobacterium tuberculosis, Mycoplasma hyopneumoniae, Myxococcus xanthus, Neosartorya fischeri, Oryctolagus cuniculus, Oryza sativa, Penicillium cyclopium, Phlebotomus papatasi, Pseudomonas aeruginosa, Pseudomonasfluorescens, Pseudomonasfragi, Pseudomonas sp, Rattus norvegicus, Rhizomucor miehei, Rhizopus oryzae, Rhizopus stolonifer, Ricinus communis, Samia cynthia ricini, Schizosaccharomyces pombe, Serratia marcescens, Spermophilus tridecemlineatus, Staphylococcus simulans, Staphylococcus xylosus, Sulfolobus solfataricus, Sus scrofa, Thermomyces lanuginosus, Trichomonas vaginalis, Vibrio harveyi, Xenopus laevis, Yarrowia lipolytica, or a combination thereof.

In some embodiments, the lipase comprises a themophilic lipase derived from Acinetobacter calcoaceticus, Acinetobacter sp., Bacillus sphaericus, Bacillus stearothermophilus, Bacillus thermocatenulatus, Bacillus thermoleovorans, Candida rugosa, Candida thermophila, GeoBacillus thermoleovorans Toshki, Pseudomonas fragi, Staphylococcus xylosus, Sulfolobus solfataricus, or a combination thereof.

In some embodiments, the lipase comprises a psychrophilic lipase derived from Pseudomonas fluorescens.

In some embodiments, the lipolytic enzyme comprises a lipoprotein lipase derived from Capra hircus, Danio rerio, Felis catus, Homo sapiens, Mesocricetus auratus, Mus musculus, Oncorhynchus mykiss, Pagrus major, Papio Anubis, Rattus norvegicus, Sparus aurata, Sus scrofa, Thunnus orientalis, or a combination thereof.

In some embodiments, the lipolytic enzyme comprises an acylglycerol lipase derived from Bacillus sp., Danio rerio, Homo sapiens, Leishmania infantum, Mus musculus, Mycobacterium tuberculosis, Penicillium camembertii, Rattus norvegicus, Solanum tuberosum, or a combination thereof.

In some embodiments, the lipolytic enzyme comprises a hormone sensitive lipase derived from Bos Taurus, Homo sapiens, Mus musculus, Rattus norvegicus, Spermophilus tridecemlineatus, Sus scrofa, Tetrahymena thermophila, or a combination thereof.

In some embodiments, the lipolytic enzyme comprises a phospholipase A₁ derived from Arabidopsis, Aspergillus oryzae, Bos Taurus, Brassica rapa, Caenorhabditis elegans, Capsicum annuum, Danio rerio, Homo sapiens, Mus musculus, Nicotiana tabacum, Polistes annularis, Polybia paulista, Rattus norvegicus, Serratia sp., Vespula vulgaris, or a combination thereof.

In some embodiments, the lipolytic enzyme comprises a phospholipase A₂ derived from Acanthaster planci, Adamsia carciniopado, Aedes aegypti, Aeropyrum pernix, Aipysurus eydouxii, Apis mellifera, Arabidopsis thaliana, Aspergillus nidulans, Austrelaps superbus, Bitis gabonica, Bos taurus, Bothriechis schlegelii, Bothropsjararacussu, BrachyDanio rerio, Bungarus caeruleus, Bungarus fasciatus, Canis familiaris, Cavia sp., Cerrophidion godmani, Chlamydomonas reinhardtii, Chrysophrys major, Crotalus viridis viridis, Daboia russellii, Danio rerio, Drosophila melanogaster, Echis carinatus, Echis ocellatus, Echis pyramidum leakeyi, Emericella nidulans, Equus caballus, Gallus gallus, Homo sapiens, Lapemis hardwickii, Laticauda semifasciata, Micrurus corallines, Mus musculus, Mytilus edulis, Naja kaouthia, Naja naja, Naja naja sputatrix, Nicotiana tabacum, Ophiophagus hannah, Ornithodoros parkeri, Oryctolagus cuniculus, Pagrus major, Patiria pectinifera, Polyandrocarpa misakiensis, Protobothrops mucrosquamatus, Rattus norvegicus, Sistrurus catenatus tergeminus, Trimeresurus borneensis, Trimeresurusflavoviridis, Trimeresurus gracilis, Trimeresurus gram ineus, Trim eresurus okinavensis, Trimeresurus puniceus, Trimeresurus stejnegeri, Tuber borchii, Urticina crassicornis, Vipera russelli siamensis, Xenopus laevis, Xenopus tropicalis, or a combination thereof.

In some embodiments, the phospholipase A₂ comprises a thermophilic phospholipase A₂ derived from Aeropyrum pernix.

In some embodiments, the lipolytic enzyme comprises a phospholipase C derived from Aedes aegypti, Aplysia californica, Arabidopsis thaliana, Asterina miniata, Bacillus cereus, Bacillus thuringiensis, Bos taurus, Caenorhabditis elegans, Chaetopterus pergamentaceus, Chlamydomonas reinhardtii, Coturnix japonica, Danio rerio, Dictyostelium discoideum, Drosophila melanogaster, Gallus gallus, Homarus americanus, Homo sapiens, Loligo pealei, Lytechinus pictus, Meleagris gallopavo, Misgurnus mizolepis, Mus musculus, Nicotiana tabacum, Oryza sativa, Oryzias latipes, Petunia inflate, Pichia stipitis, Pisum sativum, Plasmodium falciparum, Rattus norvegicus, Strongylocentrotus purpuratus, Sus scrofa, Torenia fournieri, Toxoplasma gondii, Watasenia scintillans, Xenopus laevis, Zea mays, or a combination thereof.

In some embodiments, the phospholipase C comprises a thermophilic phospholipase C derived from Bacillus cereus.

In some embodiments, the lipolytic enzyme comprises a phospholipase D derived from Aedes aegypti, Arabidopsis thaliana, Arachis hypogaea, Bos taurus, Brassica oleracea, Caenorhabditis elegans, Cricetulus griseus, Cucumis melo var. inodorus, Cucumis sativus, Dictyostelium discoideum, Drosophila melanogaster, Emericella nidulans, Fragaria ananassa, Gossypium hirsutum, Homo sapiens, Lolium temulentum, Lycopersicon esculentum, Mus musculus, Oryza sativa, Papaver somniferum, Paralichthys olivaceus, Pichia stipitis, Pimpinella brachycarpa, Rattus norvegicus, Ricinus communis, Streptoverticillium cinnamoneum, Vigna unguiculata, Vitis vinifera, Zea mays, or a combination thereof.

In some embodiments, the lipolytic enzyme comprises a phosphoinositide phospholipase C derived from Arabidopsis thaliana, Aspergillus clavatus, Aspergillus fumigatus, Brassica napus, Homo sapiens, Leishmania infantum, Mus musculus, Neosartoryafischeri, Physcomitrella patens, Pichia stipitis, Rattus norvegicus, Toxoplasma gondii, Trypanosoma brucei, Vigna unguiculata, Xenopus tropicalis, Zea mays, or a combination thereof.

In some embodiments, the lipolytic enzyme comprises a phosphatidate phosphatase derived from Saccharomyces cerevisiae, or a combination thereof.

In some embodiments, the lipolytic enzyme comprises a lysophospholipase derived from Aedes aegypti, Argas monolakensis, Aspergillus clavatus, Aspergillusfumigatus, Bos Taurus, Cavia porcellus, Clonorchis sinensis, Danio rerio, Dictyostelium discoideum, Emericella nidulans, Giardia lamblia, Homo sapiens, Monodelphis domestica, Mus musculus, Neosartoryafischeri, Pichia jadinii, Pichia stipitis, Rattus norvegicus, Schistosoma japonicum, Schizosaccharomyces pombe, Sclerotinia sclerotiorum, Xenopus tropicalis, or a combination thereof.

In some embodiments, the lipolytic enzyme comprises a sterol esterase derived from Candida rugosa, Homo sapiens, Melanocarpus albomyces, Rattus norvegicus, or a combination thereof.

In some embodiments, the lipolytic enzyme comprises a galactolipase derived from Homo sapiens, Solanum tuberosum, Vigna unguiculata, or a combination thereof.

In some embodiments, the lipolytic enzyme comprises a sphingomyelin phosphodiesterase derived from Bacillus cereus, Homo sapiens, Pseudomonas sp., or a combination thereof.

In some embodiments, the lipolytic enzyme comprises a ceramidase derived from Homo sapiens, Pseudomonas, or a combination thereof.

In some embodiments, the lipolytic enzyme comprises a retinyl palmitate esterase derived from Bos Taurus.

In some embodiments, the lipolytic enzyme comprises a cutinase derived from Fusarium solani pisi, Monilinia fructicola, Pseudomonas putida, or a combination thereof.

In some embodiments, the active enzyme comprises a mesophilic enzyme, a psychrophilic enzyme, a thermophilic enzyme, a halophilic enzyme, or a combination thereof.

In some embodiments, the active enzyme comprises a combination of enzymes.

In some embodiments, the thermophilic enzyme is derived from a thermophilic organism.

In some embodiments, the thermophilic organism comprises Acidianus, Archaeoglobus, Desulfurococcus, Hyperthermus, Metallosphaera, Methanobacterium, Methanococcus, Methanohalobium, Methanosarcina, Methanothermus, Methanosaeta, Methanothrix, Pyrobaculum, Pyrococcus, Pyrodictium, Staphylothermus, Sulfolobus, Thermococcus, Thermofilum, Thermoproteus, Clostridium, Desulfotomaculum, Rubrobacter, Saccharococcus, Sphaerobacter, Thermacetogenium, Thermoanaerobacter, Thermoanaerobium, or a combination thereof.

In some embodiments, the psychrophilic enzyme is derived a psychrophilic organism.

In some embodiments, the psychrophilic organism comprises Moritella, Leifsonia aurea, Methanococcoides burtonii, or a combination thereof.

In some embodiments, the halophilic enzyme is derived a halophilic organism.

In some embodiments, the halophilic organism comprises Halobacterium, Halococcus, Haloferax, Halogeometricum, Haloterrigena, Halorubrum, Haloarcula, or a combination thereof.

In some embodiments, the coating comprises a stimulator of enzyme activity.

In some embodiments, the stimulator comprises a co-lipase, an apolipoprotein, sodium taurocholate, bile salts, Ca²⁺, Fe²⁺, Fe³⁺, K⁺, Mg²⁺, Mn²⁺, Sr²⁺, Zn²⁺, dimethylsulfoxide, methanol, p-xylene, n-decane, a detergent, or a combination thereof.

In some embodiments, the active enzyme comprises an immobilization carrier.

In some embodiments, the immobilization carrier comprises a reverse micelle, zeolite, Celite Hyflo Supercel, a resin, diatomaceous earth, a polyurethane foam particle, macroporous polypropylene Accurel® EP 100, a macroporous anionic resin bead, polypropylene membrane, acrylic membrane, nylon membrane, cellulose ester membrane, polyvinylidene difuoride membrane, filter paper, teflon membrane, ceramic membrane, a macroporous packing particulate, polyamide, cellulose hollow fibre, a polypropylene membrane pretreated with a blocked copolymer, an immunoglobin, agarose, a gel, or a combination thereof.

In some embodiments, the active enzyme comprises a purified active enzyme.

In some embodiments, the active enzyme comprises a cell-based particulate material.

In some embodiments, the cell-based particulate material comprises a cell wall, a test, a frustule, a pellicle, a viral proteinaceous outer coat, or a combination thereof.

In some embodiments, the cell-based material comprises a multicellular-based particulate material.

In some embodiments, the cell-based particulate material comprises a microorganism-based particulate material.

In some embodiments, the cell-based particulate material is a whole cell particulate material.

In some embodiments, the cell-based particulate material is a cell fragment particulate material.

In some embodiments, the active enzyme is prepared in a material that is attenuated.

In some embodiments, the active enzyme is prepared in a material that is sterilized.

In some embodiments, the active enzyme comprises a petroleum lipolytic enzyme.

In some embodiments, the active enzyme comprises a plurality of petroleum lipolytic enzymes.

In some embodiments, the petroleum lipolytic enzyme is derived from an organism that degrades a petroleum lipid.

In some embodiments, the petroleum lipolytic enzyme is derived from Azoarcus, Blastochloris, Burkholderia, Dechloromonas, Desulfobacterium, Desulfobacula, Geobacter, Mycobacterium, Pseudomonas, Rhodococcus, Sphingomonas, Thauera, Vibrio, or a combination thereof.

In some embodiments, the petroleum lipolytic enzyme is derived from Azoarcus sp. strain EB1, Azoarcus sp. strain T, Azoarcus tolulyticus Td15, Azoarcus tolulyticus To 14, Blastochloris sulfoviridis ToP1, Blastochloris ToP1, Burkholderia sp. strain RP007, Dechloromonas sp. strain JJ, Dechloromonas sp. strain RCB, Desulfobacterium cetonicum strain AK-01, Desulfobacterium cetonicum strain Hxd3, Desulfobacterium cetonicum strain mXyS1, Desulfobacterium cetonicum strain NaphS2, Desulfobacterium cetonicum strain oXyS1, Desulfobacterium cetonicum strain Pnd3, Desulfobacterium cetonicum strain PRTOL1, Desulfobacterium cetonicum strain TD3, Desulfobacterium cetonicum, Desulfobacula toluolica To12, Geobacter 7210 metallireducens GS15, Geobacter grbiciae TACP-2T, Geobacter grbiciae TACP-5, Geobacter metallireducens GS15, Mycobacterium sp. strain PYR-1, Pseudomonas putida NCIB9816, Pseudomonas putida OUS82, Pseudomonas sp. strain C18, Pseudomonas sp. strain EbN1, Pseudomonas sp. strain HdN1, Pseudomonas sp. strain H×N1, Pseudomonas sp. strain M3, Pseudomonas sp. strain mXyN1, Pseudomonas sp. strain NAP-3, Pseudomonas sp. strain OcN1, Pseudomonas sp. strain PbN1, Pseudomonas sp. strain pCyN1, Pseudomonas sp. strain pCyN2, Pseudomonas sp. strain T3, Pseudomonas sp. strain ToN1, Pseudomonas stutzeri AN10, Rhodococcus sp. strain 124, Sphingomonas paucimobilis var. EPA505, Thauera aromatica K172, Thauera aromatica T1, Vibrio sp. strain NAP-4, or a combination thereof.

In some embodiments, the active enzyme comprises about 0.1% to about 80% of the coating by weight or volume.

In some embodiments, the active enzyme comprises about 1% to about 80% of the coating by weight or volume.

In some embodiments, the active enzyme comprises about 2% to about 80% of the coating by weight or volume.

In some embodiments, the active enzyme comprises about 3% to about 80% of the coating by weight or volume.

In some embodiments, the active enzyme comprises about 4% to about 80% of the coating by weight or volume.

In some embodiments, the active enzyme comprises about 5% to about 80% of the coating by weight or volume.

In some embodiments, the active enzyme comprises about 6% to about 80% of the coating by weight or volume.

In some embodiments, the active enzyme comprises about 7% to about 80% of the coating by weight or volume.

In some embodiments, the active enzyme comprises about 8% to about 80% of the coating by weight or volume.

In some embodiments, the active enzyme comprises about 9% to about 80% of the coating by weight or volume.

In some embodiments, the active enzyme comprises about 10% to about 80% of the coating by weight or volume.

In some embodiments, the active enzyme comprises a particulate material.

In some embodiments, the average wet molecular weight or dry molecular weight of a primary particle of the particulate material is about 50 kDa to about 1.5×10¹⁴ kDa.

In some embodiments, the average wet molecular weight or dry molecular weight of a primary particle of the particulate material is about 151 kDa to about 1.5×10¹⁴ kDa.

In some embodiments, the average wet molecular weight or dry molecular weight of a primary particle of the particulate material is about 241 kDa to about 1.5×10¹⁴ kDa.

In some embodiments, the average wet molecular weight or dry molecular weight of a primary particle of the particulate material is about 482 kDa to about 1.5×10¹⁴ kDa.

In some embodiments, the average wet molecular weight or dry molecular weight of a primary particle of the particulate material is about 753 kDa to about 1.5×10¹⁴ kDa.

In some embodiments, the average wet molecular weight or dry molecular weight of a primary particle of the particulate material is about 1,000 kDa to about 1.5×10¹⁴ kDa.

In some embodiments, the average wet molecular weight or dry molecular weight of a primary particle of the particulate material is about 1,506 kDa to about 1.5×10¹⁴ kDa.

In some embodiments, the average wet molecular weight or dry molecular weight of a primary particle of the particulate material is about 2,108 kDa to about 1.5×10¹⁴ kDa.

In some embodiments, the average wet molecular weight or dry molecular weight of a primary particle of the particulate material is about 3,613 kDa to about 1.5×10¹⁴ kDa.

In some embodiments, the average wet molecular weight or dry molecular weight of a primary particle of the particulate material is about 4,818 kDa to about 1.5×10¹⁴ kDa.

In some embodiments, the average wet molecular weight or dry molecular weight of a primary particle of the particulate material is about 6,022 kDa to about 1.5×10¹⁴ kDa.

In some embodiments, the average active enzyme content per primary particle of the particulate material is about 0.0000001% to about 100%.

In some embodiments, the average active enzyme content per primary particle of the particulate material is about 0.001% to about 100%.

In some embodiments, the average active enzyme content per primary particle of the particulate material is about 0.1% to about 100%.

In some embodiments, the average active enzyme content per primary particle of the particulate material is about 1.0% to about 100%.

In some embodiments, the average active enzyme content per primary particle of the particulate material is about 2.0% to about 100%.

In some embodiments, the average active enzyme content per primary particle of the particulate material is about 5.0% to about 100%.

In some embodiments, the average active enzyme content per primary particle of the particulate material is about 7.5% to about 100%.

In some embodiments, the average active enzyme content per primary particle of the particulate material is about 10.0% to about 100%.

In some embodiments, the average active enzyme content per primary particle of the particulate material is about 20.0% to about 100%.

In some embodiments, the average active enzyme content per primary particle of the particulate material is about 30.0% to about 100%.

In some embodiments, the coating is about 5 um to about 5000 um thick upon a surface.

In some embodiments, the coating is about 15 um to about 500 um thick upon a surface.

In some embodiments, the coating is about 15 um to about 150 um thick upon a surface.

In some embodiments, the coating comprises a paint.

In some embodiments, the coating comprises a clear coating.

In some embodiments, the clear coating comprises a lacquer, a varnish, a shellac, a stain, a water repellent coating, or a combination thereof.

In some embodiments, the coating comprises a multicoat system.

In some embodiments, the multicoat system comprises 2 to 10 layers.

In some embodiments, one layer of the multicoat system comprises the active enzyme.

In some embodiments, a plurality of layers of the multicoat system comprise the active enzyme.

In some embodiments, at least one layer of said plurality of layers comprises a different preparation of the active enzyme than at least a second layer of said plurality of layers that comprises the active enzyme.

In some embodiments, each layer of the multicoat system is coating is about 5 um to about 5000 um thick upon a surface.

In some embodiments, each layer of the multicoat system is coating is about 15 um to about 500 um thick upon a surface.

In some embodiments, each layer of the multicoat system is coating is about 15 um to about 150 um thick upon a surface.

In some embodiments, the multicoat system comprises a sealer, a water repellent, a primer, an undercoat, a topcoat, or a combination thereof.

In some embodiments, the multicoat system comprises a topcoat.

In some embodiments, the topcoat comprises the active enzyme.

In some embodiments, the coating is a coating that is capable of film formation.

In some embodiments, film formation occurs between about −10° C. to about 40° C.

In some embodiments, film formation occurs at baking conditions.

In some embodiments, baking conditions is between about 40° C. and about 50° C.

In some embodiments, baking conditions is between about 40° C. and about 65° C.

In some embodiments, baking conditions is between about 40° C. and about 110° C.

In some embodiments, the coating comprises a volatile component and a non-volatile component.

In some embodiments, the coating undergoes film formation by loss of part of the volatile component.

In some embodiments, the volatile component comprises a volatile liquid component.

In some embodiments, the volatile liquid component comprises a solvent, a thinner, a diluent, or a combination thereof.

In some embodiments, the non-volatile component comprises a binder, a colorant, a plasticizer, a coating additive, or a combination thereof.

In some embodiments, film formation occurs by crosslinking of a binder.

In some embodiments, film formation occurs by crosslinking of a plurality of binders.

In some embodiments, film formation occurs by irradiating the coating.

In some embodiments, the coating produces a self-cleaning film.

In some embodiments, the coating produces a temporary film.

In some embodiments, the temporary film has a poor resistance to a coating remover.

In some embodiments, the temporary film has a poor abrasion resistance, a poor solvent resistance, a poor water resistance, a poor weathering property, a poor adhesion property, a poor microorganism/biological resistance property, or a combination thereof.

In some embodiments, the coating is a non-film forming coating.

In some embodiments, the non-film forming coating comprises a non-film formation binder.

In some embodiments, the non-film forming coating comprises a coating component in a concentration that is insufficient to produce a solid film.

In some embodiments, the coating component comprises a binder that contributes to thermoplastic film formation.

In some embodiments, the coating component contributes to thermosetting film formation.

In some embodiments, the coating component comprises a binder, catalyst, initiator, or combination thereof.

In some embodiments, the coating component has a concentration of about 0%.

In some embodiments, the coating comprises architectural coating.

In some embodiments, the coating comprises an architectural wood coating, an architectural masonry coating, an architectural artist's coating, an architectural plastic coating, an architectural metal coating, or a combination thereof.

In some embodiments, the coating has a pot life of at least 12 months at about −10° C. to about 40° C.

In some embodiments, the coating undergoes film formation between about −10° C. to about 40° C.

In some embodiments, the coating comprises an automotive coating, a can coating, a sealant coating, or a combination thereof.

In some embodiments, the coating undergoes film formation at baking conditions.

In some embodiments, the coating comprises a chemical agent resistant coating, a camouflage coating, a pipeline coating, a traffic marker coating, an aircraft coating, a nuclear power plant coating, or a combination thereof.

In some embodiments, the coating comprises a plastic coating.

In some embodiments, the coating comprises a water-borne coating.

In some embodiments, the water-borne coating is a latex coating.

In some embodiments, the water-borne coating has a density of about 1.20 kg/L to about 1.50 kg/L.

In some embodiments, the coating comprises a solvent-borne coating.

In some embodiments, the solvent-borne coating has a density of about 0.90 kg/L to about 1.2 kg/L.

In some embodiments, the coating has a low-shear viscosity of about 100 P to about 3000 P.

In some embodiments, the coating has a low-shear viscosity of about 100 P to about 1000 P.

In some embodiments, the coating has a medium-shear viscosity of about 60 Ku and about 140 Ku.

In some embodiments, the coating has a medium-shear viscosity of about 72 Ku to about 95 Ku.

In some embodiments, the coating has a high-shear viscosity of about 0.5 P to about 2.5 P.

In some embodiments, the coating comprises a binder, a liquid component, a colorant, an additive, or a combination thereof.

In some embodiments, the coating comprises a binder.

In some embodiments, the binder comprises a thermoplastic binder, a thermosetting binder, or a combination thereof.

In some embodiments, the coating comprises a thermoplastic binder.

In some embodiments, the coating is a coating capable of producing a film by thermoplastic film formation.

In some embodiments, the coating comprises a thermosetting binder.

In some embodiments, the coating is a coating capable of producing a film by thermosetting film formation.

In some embodiments, the binder comprises an oil-based binder.

In some embodiments, the oil-based binder comprises an oil, an alkyd, an oleoresinous binder, a fatty acid epoxide ester, or a combination thereof.

In some embodiments, the coating produces a layer about 15 um to about 25 μm thick upon the vertical surface or about 15 um to about 40 μm thick upon the horizontal surface.

In some embodiments, the binder comprises a polyester resin.

In some embodiments, the polyester resin comprises a hydroxy-terminated polyester or a carboxylic acid-terminated polyester.

In some embodiments, the coating comprises a urethane, an amino resin, or a combination thereof.

In some embodiments, the binder comprises a modified cellulose.

In some embodiments, the modified cellulose comprises a cellulose ester or a nitrocellulose.

In some embodiments, the coating comprises an amino binder, an acrylic binder, a urethane binder, or a combination thereof.

In some embodiments, the binder comprises a polyamide.

In some embodiments, the coating comprises an epoxide.

In some embodiments, the binder comprises an amino resin.

In some embodiments, the coating comprises an acrylic binder, an alkyd resin, a polyester binder, or a combination thereof.

In some embodiments, the binder comprises a urethane binder.

In some embodiments, the coating comprises a polyol, an amine, an epoxide, a silicone, a vinyl, a phenolic, a triacrylate, or a combination thereof.

In some embodiments, the binder comprises a phenolic resin.

In some embodiments, the coating comprises an alkyd resin, an amino resin, a blown oil, an epoxy resin, a polyamide, a polyvinyl resin, or a combination thereof.

In some embodiments, the binder comprises an epoxy resin.

In some embodiments, the coating comprises an amino resin, a phenolic resin, a polyamide, a ketimine, an aliphatic amine, or a combination thereof.

In some embodiments, the epoxy resin comprises a cycloaliphatic epoxy binder.

In some embodiments, the coating comprises a polyol.

In some embodiments, the binder comprises a polyhydroxyether binder.

In some embodiments, the coating comprises an epoxide, a polyurethane comprising an isocyanate moiety, an amino resin, or a combination thereof.

In some embodiments, the binder comprises an acrylic resin.

In some embodiments, the coating comprises an epoxide, a polyurethane comprising an isocyanate moiety, an amino resin, or a combination thereof.

In some embodiments, the binder comprises a polyvinyl binder

In some embodiments, the coating comprises an alkyd, a urethane, an amino-resin, or a combination thereof.

In some embodiments, the binder comprises a rubber resin.

In some embodiments, the rubber resin comprises a chlorinated rubber resin, a synthetic rubber resin, or a combination thereof.

In some embodiments, the coating comprises an acrylic resin, an alkyd resin, a bituminous resin, or a combination thereof.

In some embodiments, the binder comprises a bituminous binder.

In some embodiments, the coating comprises an epoxy resin.

In some embodiments, the binder comprises a polysulfide binder.

In some embodiments, the coating comprises a peroxide, a binder comprising an isocyanate moiety, or a combination thereof.

In some embodiments, the binder comprises a silicone binder.

In some embodiments, the coating comprises an organic binder.

In some embodiments, the coating comprises a liquid component.

In some embodiments, the liquid component comprises a solvent, a thinner, a diluent, a plasticizer, or a combination thereof.

In some embodiments, the liquid component comprises a liquid organic compound, an inorganic compound, water, or a combination thereof.

In some embodiments, the liquid component comprises a liquid organic compound.

In some embodiments, the liquid organic compound comprises a hydrocarbon, an oxygenated compound, a chlorinated hydrocarbon, a nitrated hydrocarbon, a miscellaneous organic liquid, a plasticizer, or a combination thereof.

In some embodiments, the liquid organic compound comprises a hydrocarbon.

In some embodiments, the hydrocarbon comprises an aliphatic hydrocarbon, a cycloaliphatic hydrocarbon, a terpene, an aromatic hydrocarbon, or a combination thereof.

In some embodiments, the hydrocarbon comprises a petroleum ether, pentane, hexane, heptane, isododecane, a kerosene, a mineral spirit, a VMP naphtha, cyclohexane, methylcyclohexane, ethylcyclohexane, tetrahydronaphthalene, decahydronaphthalene, wood terpentine oil, pine oil, α-pinene, β-pinene, dipentene, D-limonene, benzene, toluene, ethylbenzene, xylene, cumene, a type I high flash aromatic naphtha, a type II high flash aromatic naphtha, mesitylene, pseudocumene, cymol, styrene, or a combination thereof.

In some embodiments, the liquid organic compound comprises an oxygenated compound.

In some embodiments, the oxygenated compound comprises an alcohol, an ester, a glycol ether, a ketone, an ether, or a combination thereof.

In some embodiments, the oxygenated compound comprises methanol, ethanol, propanol, isopropanol, 1-butanol, isobutanol, 2-butanol, tert-butanol, amyl alcohol, isoamyl alcohol, hexanol, methylisobutylcarbinol, 2-ethylbutanol, isooctyl alcohol, 2-ethylhexanol, isodecanol, cylcohexanol, methylcyclohexanol, trimethylcyclohexanol, benzyl alcohol, methylbenzyl alcohol, furfuryl alcohol, tetrahydrofurfuryl alcohol, diacetone alcohol, trimethylcyclohexanol, methyl formate, ethyl formate, butyl formate, isobutyl formate, methyl acetate, ethyl acetate, propyl acetate, isopropyl acetate, butyl acetate, isobutyl acetate, sec-butyl acetate, amyl acetate, isoamyl acetate, hexyl acetate, cyclohexyl acetate, benzyl acetate, methyl glycol acetate, ethyl glycol acetate, butyl glycol acetate, ethyl diglycol acetate, butyl diglycol acetate, 1-methoxypropyl acetate, ethoxypropyl acetate, 3-methoxybutyl acetate, ethyl 3-ethoxypropionate, isobutyl isobutyrate, ethyl lactate, butyl lactate, butyl glycolate, dimethyl adipate, glutarate, succinate, ethylene carbonate, propylene carbonate, butyrolactone, methyl glycol, ethyl glycol, propyl glycol, isopropyl glycol, butyl glycol, methyl diglycol, ethyl diglycol, butyl diglycol, ethyl triglycol, butyl triglycol, diethylene glycol dimethyl ether, methoxypropanol, isobutoxypropanol, isobutyl glycol, propylene glycol monoethyl ether, 1-isopropoxy-2-propanol, propylene glycol mono-n-propyl ether, propylene glycol n-butyl ether, methyl dipropylene glycol, methoxybutanol, acetone, methyl ethyl ketone, methyl propyl ketone, methyl isopropyl ketone, methyl butyl ketone, methyl isobutyl ketone, methyl amyl ketone, methyl isoamyl ketone, diethyl ketone, ethyl amyl ketone, dipropyl ketone, diisopropyl ketone, cyclohexanone, methylcylcohexanone, trimethylcyclohexanone, mesityl oxide, diisobutyl ketone, isophorone, diethyl ether, diisopropyl ether, dibutyl ether, di-sec-butyl ether, methyl tert-butyl ether, tetrahydrofuran, 1,4-dioxane, metadioxane, or a combination thereof.

In some embodiments, the liquid organic compound comprises a chlorinated hydrocarbon.

In some embodiments, the chlorinated hydrocarbon comprises methylene chloride, trichloromethane, tetrachloromethane, ethyl chloride, isopropyl chloride, 1,2-dichloroethane, 1,1,1-trichloroethane, trichloroethylene, 1,1,2,2-tetrachlorethane, 1,2-dichloroethylene, perchloroethylene, 1,2-dichloropropane, chlorobenzene, or a combination thereof.

In some embodiments, the liquid organic compound comprises a nitrated hydrocarbon.

In some embodiments, the nitrated hydrocarbon comprises a nitroparaffin, N-methyl-2-pyrrolidone, or a combination thereof.

In some embodiments, the liquid organic compound comprises a miscellaneous organic liquid.

In some embodiments, the miscellaneous organic liquid comprises carbon dioxide; acetic acid, methylal, dimethylacetal, N,N-dimethylformamide, N,N-dimethylacetamide, dimethylsulfoxide, tetramethylene suflone, carbon disulfide, 2-nitropropane, N-methylpyrrolidone, hexamethylphosphoric triamide, 1,3-dimethyl-2-imidazolidinone, or a combination thereof.

In some embodiments, the liquid organic compound comprises a plasticizer.

In some embodiments, the plasticizer comprises di(2-ethylhexyl) azelate; di(butyl) sebacate; di(2-ethylhexyl) phthalate; di(isononyl) phthalate; dibutyl phthalate; butyl benzyl phthalate; di(isooctyl) phthalate; di(idodecyl) phthalate; tris(2-ethylhexyl) trimellitate; tris(isononyl) trimellitate; di(2-ethylhexyl) adipate; di(isononyl) adipate; acetyl tri-n-butyl citrate; an epoxy modified soybean oil; 2-ethylhexyl epoxytallate; isodecyl diphenyl phosphate; tricresyl phosphate; isodecyl diphenyl phosphate; tri-2-ethylhexyl phosphate; an adipic acid polyester; an azelaic acid polyester; a bisphenoxyethylformal, or a combination thereof.

In some embodiments, the plasticizer comprises an adipate, an azelate, a citrate, a chlorinated plasticizer, an epoxide, a phosphate, a sebacate, a phthalate, a polyester, a trimellitate, or a combination thereof.

In some embodiments, the liquid component comprises an inorganic compound.

In some embodiments, the inorganic compound comprises ammonia, hydrogen cyanide, hydrogen fluoride, hydrogen cyanide, sulfur dioxide, or a combination thereof.

In some embodiments, the liquid component comprises water.

In some embodiments, the liquid component further comprises methanol, ethanol, propanol, isopropyl alcohol, tert-butanol, ethylene glycol, methyl glycol, ethyl glycol, propyl glycol, butyl glycol, ethyl diglycol, methoxypropanol, methyldipropylene glycol, dioxane, tetrahydrorfuran, acetone, diacetone alcohol, dimethylformamide, dimethyl sulfoxide, ethylbenzene, tetrachloroethylene, p-xylene, toluene, diisobutyl ketone, tricholorethylene, trimethylcyclohexanol, cyclohexyl acetate, dibutyl ether, trimethylcyclohexanone, 1,1,1-tricholoroethane, hexane, hexanol, isobutyl acetate, butyl acetate, isophorone, nitropropane, butyl glycol acetate, 2-nitropropane, methylene chloride, methyl isobutyl ketone, cyclohexanone, isopropyl acetate, methylbenzyl alcohol, cyclohexanol, nitroethane, methyl tert-butyl ether, ethyl acetate, diethyl ether, butanol, butyl glycolate, isobutanol, 2-butanol, propylene carbonate, ethyl glycol acetate, methyl acetate, methyl ethyl ketone, or a combination thereof.

In some embodiments, the coating comprises a colorant.

In some embodiments, the colorant comprises a pigment, a dye, or a combination thereof.

In some embodiments, the colorant comprises a pigment.

In some embodiments, the active enzyme comprises a particulate material that comprises about 0.000001% to about 100% of the pigment.

In some embodiments, the pigment volume concentration of the coating is about 20% to about 70%.

In some embodiments, the pigment comprises a corrosion resistance pigment, a camouflage pigment, a color property pigment, an extender pigment, or a combination thereof.

In some embodiments, the pigment comprises a corrosion resistance pigment.

In some embodiments, the corrosion resistance pigment comprises aluminum flake, aluminum triphosphate, aluminum zinc phosphate, ammonium chromate, barium borosilicate, barium chromate, barium metaborate, basic calcium zinc molybdate, basic carbonate white lead, basic lead silicate, basic lead silicochromate, basic lead silicosulfate, basic zinc molybdate, basic zinc molybdate-phosphate, basic zinc molybdenum phosphate, basic zinc phosphate hydrate, bronze flake, calcium barium phosphosilicate, calcium borosilicate, calcium chromate, calcium plumbate, calcium strontium phosphosilicate, calcium strontium zinc phosphosilicate, dibasic lead phosphite, lead chromosilicate, lead cyanamide, lead suboxide, lead sulfate, mica, micaceous iron oxide, red lead, steel flake, strontium borosilicate, strontium chromate, tribasic lead phophosilicate, zinc borate, zinc borosilicate, zinc chromate, zinc dust, zinc hydroxy phosphite, zinc molybdate, zinc oxide, zinc phosphate, zinc potassium chromate, zinc silicophosphate hydrate, zinc tetraoxylchromate, or a combination thereof.

In some embodiments, the coating is a metal surface coating.

In some embodiments, the coating comprises a primer.

In some embodiments, the pigment comprises a camouflage pigment.

In some embodiments, the camouflage pigment comprises an anthraquinone black, a chromium oxide green, the active enzyme that comprises a particulate material, or a combination thereof.

In some embodiments, the camouflage pigment reduces the ability of the coating to be detected by a devise that measures infrared radiation.

In some embodiments, the pigment comprises a color property pigment.

In some embodiments, the color property pigment comprises a black pigment, a brown pigment, a white pigment, a pearlescent pigment, a violet pigment, a blue pigment, a green pigment, a yellow pigment, an orange pigment, a red pigment, a metallic pigment, the active enzyme that comprises a particulate material, or a combination thereof.

In some embodiments, the color property pigment comprises aniline black; anthraquinone black; carbon black; copper carbonate; graphite; iron oxide; micaceous iron oxide; manganese dioxide, azo condensation, metal complex brown; antimony oxide; basic lead carbonate; lithopone; titanium dioxide; white lead; zinc oxide; zinc sulphide; titanium dioxide and ferric oxide covered mica, bismuth oxychloride crystal, dioxazine violet, carbazole Blue; cobalt blue; indanthrone; phthalocyanine blue; Prussian blue; ultramarine; chrome green; hydrated chromium oxide; phthalocyanine green; anthrapyrimidine; arylamide yellow; barium chromate; benzimidazolone yellow; bismuth vanadate; cadmium sulfide yellow; complex inorganic color; diarylide yellow; disazo condensation; flavanthrone; isoindoline; isoindolinone; lead chromate; nickel azo yellow; organic metal complex; yellow iron oxide; zinc chromate; perinone orange; pyrazolone orange; anthraquinone; benzimidazolone; BON arylamide; cadmium red; cadmium selenide; chrome red; dibromanthrone; diketopyrrolo-pyrrole; lead molybdate; perylene; pyranthrone; quinacridone; quinophthalone; red iron oxide; red lead; toluidine red; tonor; β-naphthol red; aluminum flake; aluminum non-leafing, gold bronze flake, zinc dust, stainless steel flake, nickel flake, nickel powder, or a combination thereof.

In some embodiments, the pigment comprises an extender pigment.

In some embodiments, the extender pigment comprises a barium sulphate, a calcium carbonate, a kaolin, a calcium sulphate, a silicate, a silica, an alumina trihydrate, the active enzyme that comprises a particulate material, or a combination thereof.

In some embodiments, the pigment comprises barium ferrite; borosilicate; burnt sienna; burnt umber; calcium ferrite; cerium; chrome orange; chrome yellow; chromium phosphate; cobalt-containing iron oxide; fast chrome green; gold bronze powder; luminescent; magnetic; molybdate orange; molybdate red; oxazine; oxysulfide; polycyclic; raw sienna; surface modified pigment; thiazine; thioindigo; transparent cobalt blue; transparent cobalt green; transparent iron blue; transparent zinc oxide; triarylcarbonium; zinc cyanamide; zinc ferrite; or a combination thereof.

In some embodiments, the coating comprises an additive.

In some embodiments, the additive comprises 0.000001% to 20.0% by weight, of the coating.

In some embodiments, the additive comprises an accelerator, an adhesion promoter, an antifoamer, anti-insect additive, an antioxidant, an antiskinning agent, a buffer, a catalyst, a coalescing agent, a corrosion inhibitor, a defoamer, a dehydrator, a dispersant, a drier, electrical additive, an emulsifier, a filler, a flame/fire retardant, a flatting agent, a flow control agent, a gloss aid, a leveling agent, a marproofing agent, a preservative, a silicone additive, a slip agent, a surfactant, a light stabilizer, a rheological control agent, a wetting additive, a cryopreservative, a xeroprotectant, or a combination thereof.

In some embodiments, the additive comprises a preservative.

In some embodiments, the preservative comprises an in-can preservative, an in-film preservative, or a combination thereof.

In some embodiments, the preservative comprises a biocide, a biostatic, or a combination thereof.

In some embodiments, the biocide comprises a bactericide, a fungicide, an algaecide, or a combination thereof.

In some embodiments, the preservative comprises 1-(3-chloroallyl)-3,5,7-triaza-1-azoniaadamantane chloride; 1,2-benzisothiazoline-3-one; 1,2-dibromo-2,4-dicyanobutane; 1,3-bis(hydroxymethyl)-5,5-dimethylhydantoin; 1-methyl-3,5,7-triaza-1-azonia-adamantane chloride; 2-bromo-2-nitropropane-1,3-diol; 2-(4-thiazolyl)benzimidazole; 2-(hydroxymethyl)-amino-2-methyl-1-propanol; 2(hydroxymethyl)-aminoethanol; 2,2-dibromo-3-nitrilopropionamide; 2,4,5,6-tetrachloro-isophthalonitrile; 2-mercaptobenzo-thiazole; 2-methyl-4-isothiazolin-3-one; 2-n-octyl-4-isothiazoline-3-one; 3-iodo-2-propynl N-butyl carbamate; 4,5-dichloro-2-N-octyl-3(2H)-isothiazolone; 4,4-dimethyloxazolidine; 5-chloro-2-methyl-4-isothiazolin-3-one; 5-hydroxy-methyl-1-aza-3,7-dioxabicylco(3.3.0.)octane; 6-acetoxy-2,4-dimethyl-1,3-dioxane; 7-ethyl bicyclooxazolidine; a combination of 1,2-benzisothiazoline-3-one and hexahydro-1,3,5-tris(2-hydroxyethyl)-s-triazine; a combination of 1,2-benzisothiazoline-3-one and zinc pyrithione; a combination of 2-(thiocyanomethyl-thio)benzothiozole and methylene bis(thiocyanate); a combination of 4-(2-nitrobutyl)-morpholine and 4,4′-(2-ethylnitrotrimethylene)dimorpholine; a combination of 4,4-dimethyl-oxazolidine and 3,4,4-trimethyloxazolidine; a combination of 5-chloro-2-methyl-4-isothiazolin-3-one and 2-methyl-4-isothiazolin-3-one; a combination of carbendazim and 3-iodo-2-propynl N-butyl carbamate; a combination of carbendazim, 3-iodo-2-propynl N-butyl carbamate and diuron; a combination of chlorothalonil and 3-iodo-2-propynl N-butyl carbamate; a combination of chlorothalonil and a triazine compound; a combination of tributyltin benzoate and alkylamine hydrochlorides; a combination of zinc-dimethyldithiocarbamate and zinc 2-mercaptobenzothiazole; a copper soap; a metal soap; a mercury soap; a mixture of bicyclic oxazolidines; a tin soap; an alkylamine hydrochloride; an amine reaction product; barium metaborate; butyl parahydroxybenzoate; carbendazim; copper(II) 8-quinolinolate; diiodomethyl-p-tolysulfone; dithio-2,2-bis(benzmethylamide); diuron; ethyl parahydroxybenzoate; glutaraldehyde; hexahydro-1,3,5-triethyl-s-triazine; hexahydro-1,3,5-tris(2-hydroxyethyl)-s-triazine; hydroxymethyl-5,5-dimethylhydantoin; methyl parahydroxybenzoate; N-butyl-1,2-benzisothiazolin-3-one; N-(trichloromethylthio)phthalimide; N-cyclopropyl-N-(1-dimethylethyl)-6-(methylthio)-1,3,5-triazine-2,4-diamine; N-trichloromethyl-thio-4-cyclohexene-1,2-dicarboximide; p-chloro-m-cresol; phenoxyethanol; phenylmercuric acetate; poly(hexamethylene biguanide) hydrochloride; potassium dimethyldithiocarbamate; potassium N-hydroxy-methyl-N-methyl-dithiocarbamate; propyl parahydroxybenzoate; sodium 2-pyridinethiol-1-oxide; tetra-hydro-3,5-di-methyl-2H-1,3,5-thiadiazine-2-thione; tributyltin benzoate; tributyltin oxide; tributyltin salicylate; zinc pyrithione; sodium pyrithione; copper pyrithione; zinc oxide; a zinc soap; or a combination thereof.

In some embodiments, the additive comprises a wetting additive, a dispersant, or a combination thereof.

In some embodiments, the additive comprises a combination of an unsaturated polyamine amide salt and a lower molecular weight acid; a polycarboxylic acid polymer alkylolammonium salt; a combination of a long chain polyamine amide salt and a polar acidic ester; a hydroxyfunctional carboxylic acid ester; a non-ionic wetting agent; or a combination thereof.

In some embodiments, the additive comprises a wetting additive.

In some embodiments, the wetting additive comprises an ethylene oxide molecule comprising a hydrophobic moiety; a surfactant; pine oil; a metal soap; calcium octoate; zinc octoate; aluminum stearate; zinc stearate; bis(2-ethylhexyl)sulfosuccinate; (octylphenoxy)polyethoxyethanol octylphenyl-polyethylene glycol; nonyl phenoxy poly (ethylene oxy)ethanol; ethylene glycol octyl phenyl ether; or a combination thereof.

In some embodiments, the additive comprises a dispersant.

In some embodiments, the dispersant comprises tetra-potassium pyrophosphate, a phosphate ester surfactant; a particulate material, a calcium carbonate coated with fatty acid, a modified montmorillonite clay, a caster wax, or a combination thereof.

In some embodiments, the additive comprises an antifoamer, a defoamer, or a combination thereof.

In some embodiments, the additive comprises an oil; a mineral oil; a silicon oil; a fatty acid ester; dibutyl phosphate; a metallic soap; a siloxane; a wax; an alcohol comprising six to ten carbons; a pine oil; or a combination thereof.

In some embodiments, the coating further comprises an emulsifier, a hydrophobic silica, or a combination thereof.

In some embodiments, the additive comprises a rheological control agent.

In some embodiments, the rheology control agent comprises a silicate; a montmorillonite silicate; aluminum silicate, a bentonite, magnesium silicate, a cellulose ether, a hydrogenated oil, a polyacrylate, a polyvinylpyrrolidone, a urethane, a methyl cellulose, a hydroxyethyl cellulose, hydrogenated castor oil; a hydrophobically modified ethylene oxide urethane; a titanium chelate, a zirconium chelate, the active enzyme that comprises a particulate material, or a combination thereof.

In some embodiments, the rheological control agent comprises a thickener, a viscosifier, or a combination thereof.

In some embodiments, the additive comprises a corrosion inhibitor.

In some embodiments, the corrosion inhibitor comprises a chromate, a phosphate, a molybdate, a wollastonite, a calcium ion-exchanged silica gel, a zinc compound, a borosilicate, a phosphosilicate, a hydrotalcite, or a combination thereof.

In some embodiments, said corrosion inhibitor comprises an in-can corrosion inhibitor, a flash corrosion inhibitor, or a combination thereof.

In some embodiments, the corrosion inhibitor comprises sodium nitrate, sodium benzoate, ammonium benzoate, 2-amino-2-methyl-propan-1-ol, or a combination thereof.

In some embodiments, the additive comprises a light stabilizer.

In some embodiments, the light stabilizer comprises a UV absorber, a radical scavenger, or a combination thereof.

In some embodiments, the light stabilizer comprises a UV absorber.

In some embodiments, the UV absorber comprises a hydroxybenzophenone, a hydroxyphenylbenzotriazole, a hydrozyphenyl-S-triazine, an oxalic anilide, yellow iron oxide, the active enzyme, or a combination thereof.

In some embodiments, the light stabilizer comprises a radical scavenger.

In some embodiments, the radical scavenger comprises a sterically hindered amine; bis(1,2,2,6,6,-pentamethyl-4-poperidinyl)ester, bis(2,2,6,6,-tetramethyl-1-isooctyloxy-4-piperidinyl)ester, or a combination thereof.

In some embodiments, said additive comprises a buffer.

In some embodiments, the buffer comprises a bicarbonate, a monobasic phosphate buffer, a dibasic phosphate buffer, Trizma base, a 5 zwitterionic buffer, triethanolamine, or a combination thereof.

In some embodiments, the buffer comprises a bicarbonate.

In some embodiments, the bicarbonate comprises an ammonium bicarbonate.

In some embodiments, the concentration of the buffer in the coating is about 0.000001 M to about 2.0 M.

In some embodiments, said additive comprises a cryopreservative, a xeroprotectant, or a combination thereof.

In some embodiments, the additive comprises a cryopreservative.

In some embodiments, the cryopreservative comprises glycerol, DMSO, a protein, a sugar of 4 to 10 carbons, or a combination thereof.

In some embodiments, the additive comprises a xeroprotectant.

In some embodiments, the xeroprotectant comprises glycerol, a glycol, a mineral oil, a bicarbonate, DMSO, a sugar of 4 to 10 carbons, or a combination thereof.

In some embodiments, the active enzyme comprises 0.000001% to 80%, by weight or volume, a cryopreservative, a xeroprotectant, or a combination thereof.

In some embodiments, the coating is a multi-pack coating.

In some embodiments, the multi-pack coating is stored in a two to five containers prior to application to a surface.

In some embodiments, about 0.000001% to about 100% of the active enzyme is stored in a container of the multi-pack coating, and at least one coating component is stored in another container of the multi-pack coating.

In some embodiments, the container that stores the active enzyme further stores an additional coating component.

In some embodiments, the additional coating component comprises a preservative, a wetting agent, a dispersing agent, a buffer, a liquid component, a rheological modifier, a cryopreservative, a xeroprotectant, or a combination thereof.

In some embodiments, the coating is a coating capable of being applied to a surface by a spray applicator.

In some embodiments, the active enzyme is microencapsulated.

In some embodiments, the coating comprises a pH indicator.

In some embodiments, the pH indicator is a colormetric indicator.

In some embodiments, the colormetric indicator comprises Alizarin, Alizarin S, Brilliant Yellow, Lacmoid, Neutral Red, Rosolic Red, or a combination thereof.

In some embodiments, the pH indicator is a fluorimetric indicator.

In some embodiments, the fluorimetric indicator comprises SNARF-1, BCECF, HPTS, Fluoroescein, or a combination thereof.

In some embodiments, the pH indicator is a pH indicator that undergoes a color or fluorescence change between about pH 8 to about pH 9.

In some embodiments, the lipolytic enzyme possesses phosphoric triester hydrolase activity, the ability to bind an organophosphorus compound, or a combination thereof.

In some embodiments, the lipolytic enzyme comprises a carboxylesterase.

In some embodiments, the carboxylesterase is derived from Anisopteromalus calandrae, Aphis gossypii, Homo sapiens, Myzus persicae, Rattus norvegicus, or a combination thereof.

In some embodiments, the esterase comprises a phosphoric triester hydrolase.

In some embodiments, the esterase comprises a plurality of phosphoric triester hydrolases.

In some embodiments, the phosphoric triester hydrolase comprises an aryldialkylphosphatase, a diisopropyl-fluorophosphatase, or a combination thereof.

In some embodiments, the phosphoric triester hydrolase comprises a combination of phosphoric triester hydrolases.

In some embodiments, the phosphoric triester hydrolase comprises an aryldialkylphosphatase.

In some embodiments, the aryldialkylphosphatase comprises an organophosphorus hydrolase, a human paraoxonase, an animal carboxylase, or a combination thereof.

In some embodiments, the aryldialkylphosphatase comprises an organophosphorus hydrolase.

In some embodiments, the organophosphorus hydrolase comprises an Agrobacterium radiobacter P230 organophosphate hydrolase, a Flavobacterium balustinum parathion hydrolase, a Pseudomonas diminuta phosphotriesterase, a Flavobacterium sp opd gene product, a Flavobacterium sp. parathion hydrolase opd gene product, or a combination thereof.

In some embodiments, the aryldialkylphosphatase comprises a human paraoxonase.

In some embodiments, the aryldialkylphosphatase comprises an animal carboxylase.

In some embodiments, the animal carboxylase comprises an insect carboxylase.

In some embodiments, the insect carboxylase comprises a Plodia interpunctella carboxylase, Chrysomya putoria carboxylase, Lucilia cuprina carboxylase, Musca domestica carboxylase carboxylase, or a combination thereof.

In some embodiments, the phosphoric triester hydrolase comprises a diisopropyl-fluorophosphatase.

In some embodiments, the diisopropyl-fluorophosphatase comprises an organophosphorus acid anhydrolase, a squid-type DFPase, a Mazur-type DFPase, or a combination thereof.

In some embodiments, the diisopropyl-fluorophosphatase comprises an organophosphorus acid anhydrolase.

In some embodiments, the organophosphorus acid anhydrolase comprises an Altermonas organophosphorus acid anhydrolase, a prolidase, or a combination thereof.

In some embodiments, the organophosphorus acid anhydrolase comprises an Altermonas organophosphorus acid anhydrolase.

In some embodiments, the Altermonas organophosphorus acid anhydrolase comprises an Alteromonas sp JD6.5 organophosphorus acid anhydrolase, an Alteromonas haloplanktis organophosphorus acid anhydrolase, an Altermonas undina organophosphorus acid anhydrolase, or a combination thereof.

In some embodiments, the organophosphorus acid anhydrolase comprises a prolidase.

In some embodiments, the prolidase comprises a human prolidase, a Mus musculus prolidase, a Lactobacillus helveticus prolidase, an Escherichia coli prolidase, an Escherichia coli aminopeptidase P, or a combination thereof.

In some embodiments, the diisopropyl-fluorophosphatase comprises a squid-type DFPase.

In some embodiments, the squid-type DFPase comprises a Loligo vulgaris DFPase, a Loligo pea lei DFPase, a Loligo opalescens DFPase, or a combination thereof.

In some embodiments, the diisopropyl-fluorophosphatase comprises a Mazur-type DFPase, or a combination thereof.

In some embodiments, the Mazur-type DFPase comprises a mouse liver DFPase, a hog kidney DFPase, a Bacillus stearothermophilus strain OT DFPase, an Escherichia coli DFPase, or a combination thereof.

In some embodiments, the phosphoric triester hydrolase comprises a Plesiomonas sp. strain M6 mpd gene product, a Xanthomonas sp. phosphoric triester hydrolase, a Tetrahymena phosphoric triester hydrolase, or a combination thereof.

In some embodiments, the active enzyme comprises an esterase.

In some embodiments, the active enzyme comprises a plurality of esterases.

In some embodiments, the active enzyme comprises a peptidase.

In some embodiments, the active enzyme comprises a plurality of peptidases.

In some embodiments, the peptidase comprises an alpha-amino-acyl-peptide hydrolase, a peptidyl-amino-acid hydrolase, a dipeptide hydrolase, a peptidyl peptide hydrolase, a peptidylamino-acid hydrolase, an acylamino-acid hydrolase, an aminopeptidase, a dipeptidase, a dipeptidyl-peptidase, a tripeptidyl-peptidase, a peptidyl-dipeptidase, a serine-type carboxypeptidase, a metallocarboxypeptidase, a cysteine-type carboxypeptidase, an omega peptidase, a serine endopeptidase, a cysteine endopeptidase, an aspartic endopeptidase, a metalloendopeptidase, a threonine endopeptidase, an endopeptidases of unknown catalytic mechanism, or a combination thereof.

In some embodiments, the peptidase comprises a chymotrypsin, a typsin, or a combination thereof.

In some embodiments, the esterase comprises a sulfuric ester hydrolase.

In some embodiments, the esterase comprises a plurality of sulfuric ester hydrolases.

In some embodiments, the sulfuric ester hydrolase comprises an arylsulfatase, a steryl-sulfatase, a glycosulfatase, a N-acetylgalactosamine-6-sulfatase, a choline-sulfatase, a cellulose-polysulfatase, cerebroside-sulfatase, a chondro-4-sulfatase, a chondro-6-sulfatase, a disulfoglucosamine-6-sulfatase, a N-acetylgalactosamine-4-sulfatase, an iduronate-2-sulfatase, an N-acetylglucosamine-6-sulfatase, a N-sulfoglucosamine-3-sulfatase, a monomethyl-sulfatase, a D-lactate-2-sulfatase, a glucuronate-2-sulfatase, or a combination thereof.

In some embodiments, the sulfuric ester hydrolase comprises an arylsulfatase.

Some embodiments provide a coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises an esterase.

Some embodiments provide a coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises a petroleum lipolytic enzyme.

Some embodiments provide a coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises a peptidase.

Some embodiments provide a coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises a lipolytic enzyme.

Some embodiments provide a coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises a lipolytic enzyme.

Some embodiments provide a coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises a sulfuric ester hydrolase,

Some embodiments provide an architectural coating composition, comprising an architectural coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide an architectural wood coating composition, comprising an architectural wood coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide an architectural masonry coating composition, comprising an architectural masonry coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide an architectural artist's coating composition, comprising an architectural artist's coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide an architectural plastic coating composition, comprising an architectural plastic coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide an architectural metal coating composition, comprising an architectural metal coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a clear coating composition, comprising a clear coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide an automotive coating composition, comprising an automotive coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a can coating composition, comprising a can coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a sealant coating composition, comprising a sealant coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a chemical agent resistant coating coating composition, comprising a chemical agent resistant coating coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a camouflage coating composition, comprising a camouflage coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a specification coating composition, comprising a specification coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a pipeline coating composition, comprising a pipeline coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a traffic marker coating composition, comprising a traffic marker coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide an aircraft coating composition, comprising an aircraft coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a nuclear power plant coating composition, comprising a nuclear power plant coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a marine coating composition, comprising a marine coating that comprises an active enzyme, wherein the active enzyme comprises an antimicrobial enzyme, an antifouling enzyme, a peptidase, an esterase, a petroleum lipolytic enzyme, or a combination thereof.

Some embodiments provide a water-borne coating composition, comprising a water-borne coating that comprises an active enzyme, wherein the active enzyme comprises an antimicrobial enzyme, an antifouling enzyme, a peptidase, an esterase, a petroleum lipolytic enzyme, or a combination thereof.

In some embodiments, the water-borne coating comprises an architectural coating.

Some embodiments provide a solvent-borne coating composition, comprising a solvent-borne coating that comprises an active enzyme, wherein the active enzyme comprises an antimicrobial enzyme, an antifouling enzyme, a peptidase, an esterase, a petroleum lipolytic enzyme, or a combination thereof.

In some embodiments, the solvent-borne coating comprises an architectural coating.

Some embodiments provide a powder coating composition, comprising a powder coating and an active enzyme, wherein the active enzyme comprises an antimicrobial enzyme, an antifouling enzyme, a peptidase, an esterase, a petroleum lipolytic enzyme, or a combination thereof.

Some embodiments provide a multi-pack coating composition, comprising a plurality of containers, wherein at least one container comprises an active enzyme, and wherein the coating comprises an architectural wood coating, an architectural masonry coating, an architectural artist coating, an automotive coating, a can coating, a sealant coating, a chemical agent resistant coating, a camouflage coating, a pipeline coating, a traffic marker coating, an aircraft coating, a nuclear power plant coating, a marine coating, or a combination thereof, and wherein the active enzyme comprises an antimicrobial enzyme, an antifouling enzyme, a peptidase, an esterase, a petroleum lipolytic enzyme, or a combination thereof.

Some embodiments provide a non-film forming coating, comprising a non-film forming coating and an active enzyme, wherein the active enzyme comprises an antimicrobial enzyme, an antifouling enzyme, a peptidase, an esterase, a petroleum lipolytic enzyme, or a combination thereof.

Some embodiments provide an elastomer composition, comprising an elastomer and an active enzyme comprises an active enzyme, wherein the active enzyme comprises an antimicrobial enzyme, an antifouling enzyme, a peptidase, an esterase, a petroleum lipolytic enzyme, or a combination thereof.

Some embodiments provide a filler composition, comprising a filler and an active enzyme, wherein the active enzyme comprises an antimicrobial enzyme, an antifouling enzyme, a peptidase, an esterase, a petroleum lipolytic enzyme, or a combination thereof.

Some embodiments provide an adhesive composition, comprising an adhesive and an active enzyme, wherein the active enzyme comprises an antimicrobial enzyme, an antifouling enzyme, a peptidase, an esterase, a petroleum lipolytic enzyme, or a combination thereof.

Some embodiments provide a sealant composition, comprising a sealant and an active enzyme, wherein the active enzyme comprises an antimicrobial enzyme, an antifouling enzyme, a peptidase, an esterase, a petroleum lipolytic enzyme, or a combination thereof.

Some embodiments provide a textile finish composition, comprising a textile finish and an active enzyme, wherein the active enzyme comprises an antimicrobial enzyme, an antifouling enzyme, a peptidase, an esterase, a petroleum lipolytic enzyme, or a combination thereof.

Some embodiments provide a wax, comprising a wax and an active lipolytic enzyme, wherein the active enzyme comprises an antimicrobial enzyme, an antifouling enzyme, a peptidase, an esterase, a petroleum lipolytic enzyme, or a combination thereof. Some embodiments provide a polymeric material, comprising a polymeric material an active enzyme, wherein the active enzyme comprises an antimicrobial enzyme, an antifouling enzyme, a peptidase, an esterase, a petroleum lipolytic enzyme, or a combination thereof.

Some embodiments provide a method of inhibiting microbical grown on a surface, comprising the steps of: contacting a surface contaminated with a microorganism with a coating comprising an active enzyme, wherein the active enzyme comprises an antimicrobical enzyme or antifouling enzyme. Some embodiments provide a method of cleaning a surface contaminated with a chemical and/or a microorganims, comprising the steps of: contacting a surface contaminated with a chemical and/or a microorganism with a coating comprising an active enzyme, wherein the active enzyme comprises an antimicrobial enzyme, an antifouling enzyme, an esterase, a petroleum lipolytic enzyme, a peptidase, or a combination thereof, wherein the chemical comprises an ester linkage, a peptide linkage, or a lipid.

In some embodiments, the surface is located in a kitchen or food preparation area.

Some embodiments provide a method of reducing the concentration of a chemical and/or a microorganism on a surface, comprising the steps of: applying a coating to the surface, wherein the coating comprises an architectural wood coating, an architectural masonry coating, an architectural artist coating, an automotive coating, a can coating, a sealant coating, a camouflage coating, a pipeline coating, a traffic marker coating, an aircraft coating, a nuclear power plant coating, a maring coating, or a combination thereof, and wherein the coating comprises an active enzyme, wherein the active enzyme comprises an antimicrobial enzyme, an antifouling enzyme, an esterase, a petroleum lipolytic enzyme, a peptidase, or a combination thereof, and contacting the surface with a chemical and/or microorganism, wherein the chemical comprises an ester linkage, a peptide linkage, or a lipid. In some embodiments, the step of applying to the surface a coating occurs prior to contacting the surface with the chemical and/or a microorganism. In some embodiments, the surface is located in a kitchen or food preparation area. In some embodiments, the surface is located in a marine environment. In some embodiments, the surface is located on a stove, a sink, a drain pipe, a counter top, a floor, a wall, a cabinet, an appliance, or a combination thereof. In some embodiments, the coating is formulated as an interior coating. In some embodiments, the method further comprises the step of: applying a cleaning material to the surface, and removing the chemical, a product of the reaction of the chemical with the active enzyme, or a combination thereof. In some embodiments, the cleaning material comprises a cleaning solution, a cleaning devise, a disinfectant, or a combination thereof.

Some embodiments provide a method of cleaning a surface contaminated with a chemical and/or a microorganism, comprising the steps of: contacting a surface contaminated with a chemical and/or a microorganism, with a surface treatment comprising an active enzyme, wherein the active enzyme comprises an antimicrobial enzyme, an antifouling enzyme, an esterase, a petroleum lipolytic enzyme, a peptidase, or a combination thereof, wherein the chemical comprises an ester linkage, a peptide linkage, or a lipid.

Some embodiments provide a method of reducing the concentration of a chemical and/or a microorganism on a surface, comprising the steps of: applying a surface treatment to the surface, wherein the surface treatment comprises an active enzyme, wherein the active enzyme comprises an antimicrobial enzyme, an antifouling enzyme, an esterase, a petroleum lipolytic enzyme, a peptidase, or a combination thereof, and contacting the surface with a chemical and/or microorganism, wherein the chemical comprises an ester linkage, a peptide linkage, or a lipid.

Some embodiments provide a method of preparing an enzymatically active surface treatment or polymeric material, comprising the steps of: obtaining an active enzyme, and admixing at least one component of a surface treatment or polymeric material with the active enzyme, wherein the active enzyme comprises an antimicrobial enzyme, an antifouling enzyme, a peptidase, an esterase, a petroleum lipolytic enzyme, or a combination thereof.

In some embodiments the component of the surface treatment or polymeric material comprises all non-enzyme components of the surface treatment or polymeric material.

Some embodiments provide a method of preparing an enzymatically active surface treatment or polymeric material, comprising the steps of: obtaining an active enzyme, and admixing a surface treatment or polymeric material with the active enzyme, wherein the active enzyme comprises an antimicrobial enzyme, an antifouling enzyme, a peptidase, an esterase, a petroleum lipolytic enzyme, or a combination thereof.

Some embodiments provide a surface treatment or polymeric material, prepared in accordance with the methods described herein.

Some embodiments provide a kit having component parts capable of being assembled comprising a container comprising an active enzyme, and a container comprising at least one component of a surface treatment or polymeric material, wherein the active enzyme comprises an antimicrobial enzyme, an antifouling enzyme, a peptidase, an esterase, a petroleum lipolytic enzyme, or a combination thereof.

Some embodiments provide a kit having component parts capable of being assembled comprising a container comprising an active enzyme, and a container comprising at least one surface treatment polymeric material, wherein the active enzyme comprises an antimicrobial enzyme, an antifouling enzyme, a peptidase, an esterase, a petroleum lipolytic enzyme, or a combination thereof.

Some embodiments provide an article of manufacture having enzyme activity, comprising a manufactured object, wherein at least one surface or component of the object comprises a surface treatment, wherein the surface treatment comprises an active enzyme comprising an antimicrobial enzyme, an antifouling enzyme, a peptidase, an esterase, a petroleum lipolytic enzyme, or a combination thereof. Some embodiments provide an article of manufacture having enzyme activity, comprising a manufactured object, wherein at least one surface or component of the object comprises a polymeric material, wherein the polymeric material comprises an active enzyme comprising an antimicrobial enzyme, an antifouling enzyme, a peptidase, an esterase, a petroleum lipolytic enzyme, or a combination thereof. In some embodiments the surface of the object is part of an interior or exterior component of the object.

Some embodiments provide a coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises an antimicrobial enzyme. In some aspects, the active enzyme comprises a peptidase, an esterase, a petroleum lipolytic enzyme, or a combination thereof.

### DETAILED DESCRIPTION OF THE INVENTION

For a further understanding of the nature and function, reference should now be made to the following detailed description taken in conjunction with the accompanying drawings. Detailed descriptions of the embodiments are provided herein, as well as, the best mode of carrying out and employing the present invention. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

One skilled in the art will readily appreciate that the present invention is well adapted to carry out and obtain the ends and features mentioned as well as those inherent therein. It should be understood, however, that the biomolecular compositions, compounds, coatings, paints, films, methods, procedures, and techniques described herein are presently representative of various embodiments. These techniques are intended to be exemplary, are given by way of illustration only, and are not intended as limitations on the scope. Other features will be readily apparent to one skilled in the art from the following detailed description; specific examples and claims; and various changes, substitutions, other uses and modifications that may be made to the invention disclosed herein without departing from the scope and spirit of the invention or as defined by the scope of the appended claims.

As used herein other than the claims, the terms “a,” “an,” “the,” and “said” means one or more. As used herein in the claim(s), when used in conjunction with the words “comprises” or “comprising,” the words “a,” “an,” “the,” or “said” may mean one or more than one. As used herein “another” may mean at least a second or more. As used in the claims, “about” refers to any inherent measurement error or a rounding of digits for a measured or calculated value (e.g., ratio), and thus the term “about” may be used with any value or range. Various genera and sub-genera described herein are contemplated both as individual components, as well as and mixtures and combinations that may be described in the claims as “at least one selected from,” “a mixture thereof” and/or “a combination thereof.” For example, compositions described as a coating suitable for plastic surfaces described in different sections of the specification may be claimed individually or as a combination, as they are part of the same genera of plastic coatings. All patents and publications mentioned in this specification are herein incorporated by reference to the same extent as if each individual publication was specifically and individually indicated to be incorporated by reference.

In some cases, compositions and methods described herein may be used for prophylactic protection of buildings (e.g., restaurants, household food preparation areas), equipment (e.g., a stove, a sink, a drain pipe, a counter top, a floor, a wall, a cabinet, an appliance, etc.), and personnel that contact from spills and splatters (e.g., chemical spills). For example, the compositions and methods described herein have use in aiding the cleanup of unwanted lipid spills, splatters and contamination of lipids that comprise a fatty acid, such as the fats, oils, and waxes and their commercial products (e.g., cosmetics) used systemically in daily life. In some aspects, the compositions and methods described herein may be combined with others, such as a cleaning material (e.g., water, detergent solutions, mops, sponges, power washers, etc.) and methods of application and removal of such solutions and compositions after incorporation of the contaminating material, to aid in the removal of a contaminating material (e.g., a chemical). An example of such a composition is a “surface treatment” which refers to compositions applied to a surface, and examples of such compositions specifically contemplated include a coating (e.g., a paint, a clear coat), a textile finish, a wax, elastomer, an adhesive, a filler, or a sealant, or other compositions described herein.

In some embodiments, the average weight per single particle (“primary particle”) of a biomolecular composition (e.g., a lipolytic enzyme) may be measured in “wet weight,” which refers to the weight of the particle prior to a drying or an extraction step that would remove the liquid component of a cell (e.g., the aqueous component of the cell's cytoplasm). In certain aspects, the “wet weight” of a biomolecular composition (e.g., a whole cell particulate material) that has its liquid component replaced by some other liquid (e.g., an organic solvent) may also be measured in “wet weight.” The “dry weight” refers to the average per particle weight of a biomolecular composition after the majority of the liquid component has been removed. The term “majority” refers 50% to 100%, including all intermediate ranges and combinations thereof, with the greater values (e.g., 85% to 100%) contemplated in some aspects. In general embodiments, it is contemplated that the dry weight of a biomolecular composition will typically be 5% to 30% the wet weight, including all intermediate ranges and combinations thereof, as it is usual for 70% to 95% of a cell to be water. Any technique for measuring cell or particle size, volume, density, etc. used for various insoluble particulate materials (e.g., pigments) used as coating, paint, or surface treatment components may be applied to a biomolecular composition to determine wet or dry weight values, particle size, particle density, etc. Additionally, various examples of specific techniques are described herein. Further, such measurements of cell size, shape, density, numbers, etc. is used in the art of microbiology. For example, the average number of particles, size, shape, etc. of a biomolecular composition may be microscopically determined for a given volume and weight of material, whether prepared as a “wet weight” or “dry weight material,” and the average particle weight, density, volume, etc. calculated.

Many variations of nomenclature are commonly used to refer to a specific chemical composition. Accordingly, several common alternative names may be provided herein in quotations and parentheses/brackets, or other grammatical technique, adjacent to a chemical composition's preferred designation when referred to herein. Additionally, many chemical compositions referred to herein are further identified by a Chemical Abstracts Service registration number. The Chemical Abstracts Service provides a unique numeric designation, denoted herein as “CAS No.,” for specific chemicals and some chemical mixtures, which unambiguously identifies a chemical composition's molecular structure.

In various embodiments described herein, exemplary values are specified as a range. Examples of such ranges cited herein include, for example, a size of a biomolecule, a temperature for growth and/or preparation of a microorganism, a chemical moiety's content in a coating component, a coating component's content in a coating composition and/or film, a coating component's mass, a glass transition temperature (“T_{g}”), a temperature for a chemical reaction (e.g., film formation, chemical modification of a coating component), the thickness of a coating and/or film upon a surface, etc. It will be understood that herein the phrase “including all intermediate ranges and combinations thereof” associated with a given range includes all integers and sub-ranges comprised within a cited range. For example, citation of a range “0.03% to 0.07%, including all intermediate ranges and combinations thereof” provides specific values within the cited range, such as, for example, 0.03%, 0.04%, 0.05%, 0.06%, and 0.07%, as well as various combinations of such specific values, such as, for example, 0.03%, 0.06% and 0.07%, 0.04% and 0.06%, or 0.05% and 0.07%, as well as sub-ranges such as 0.03% to 0.05%, 0.04% to 0.07%, or 0.04% to 0.06%, etc. Additionally, Example 36 provides additional descriptions of specific numeric values within a cited range. The phrase “or a combination thereof” refers to any combination (e.g., any sub-set) of a set of listed components.

### A. Biomolecules

As used herein, a “biomolecular composition” or “biomolecule composition” refers to a composition comprising a biomolecule. As used herein, a “biomolecule” refers to a compound comprising of one or more chemical moieties typically synthesized in living organisms, including but not limited to, an amino acid, a nucleotide, a polysaccharide or simple sugar, a lipid, or a combination thereof. A biomoleculetypically comprises a proteinaceous molecule. As used herein a “proteinaceous molecule” comprises a polymer formed from amino acids, such as a peptide or a polypeptide. Examples of proteinaceous molecules include an enzyme, an antibody, a receptor, a transport protein, structural protein, or a combination thereof. Examples of a peptide include inhibitory peptides of 3 to 15 amino acids, as well as peptides of 3 to 100 amino acids.

In some embodiments, a biomolecular composition comprises a cell and/or cell debris (a “cell-based” material), in contrast to a purified biomolecule (e.g., a purified enzyme). In general embodiments, a cell used in a cell-based particulate material comprises a durable structure at the cell-external environment interface, such as, for example, a cell wall, a silica based shell (“test”), a silica based exoskeleton (“frustule”), a pellicle, proteinaceous outer coat, or a combination thereof. In typical embodiments, a cell is obtained from an organism is a unicellular and/or oligocellular organism, as it is contemplated that particulate matter may be prepared from such an organism without a step to separate one or more cells from a multicellular tissue or organism (e.g., a plant) into a smaller average particle size suitable for preparation of a coating or other surface treatment.

It is contemplated that one may obtain biological materials such as viruses (e.g., bacteriophages), cells (e.g., microorganisms), tissues, and organisms (e.g., plants) from an environmental source using conventional procedures [see, for example, “Environmental Biotechnology Isolation of Biotechnological Organisms From Nature (Labeda, D. P., Ed.), 1990]. However, many live cultures, seeds, organisms, etc. of previously isolated and characterized biological materials have been conveniently cataloged and stored by public depositories and/or commercial vendors for the ease of use. Additionally, the identification of a biological material, particularly microorganisms, usually comprises characterization of suitable growth conditions for the cell, such as energy source (e.g., a digestible organic molecule), vitamin requirements, mineral requirements, pH conditions, light conditions, temperature, etc. [see, for example, “Bergey's Manual of Determinative Bacteriology Ninth Edition” (Hensyl, W. R., Ed.), 1994”; “The Yeasts—A Taxonomic Study—Fourth Revised and Enlarged Edition” (Kurtzman, C. P. and Fell, J. W., Eds.), 1998”; and “The Springer Index of Viruses” (Tidona, C. A. and Darai, G., Eds.), 2001]. Such biological materials and information about appropriate growth conditions is readily obtainable from the biological culture collection and/or commercial vendor that stores the biological material. Hundreds of such biological culture collections currently exist, and the location of a specific biological material may be identified using a database such as that maintained by the World Data Center for Microorganisms (National Institute of Genetics, WFCC-MIRCEN World Data Center for Microorganisms, 1111 Yata, Mishima, Shizuoka, 411-8540 JAPAN). Specific examples of biological culture collections referred to herein include the American Type Culture Collection (“ATCC” “; P.O. Box 1549, Manassas, Va. 20108-1549, U.S.A), the Culture Collection of Algae and Protozoa (“CCAP” “; CEH Windermere, The Ferry House, Far Sawrey, Ambleside, Cumbria LA22 0LP, United Kingdom), the Collection de l'Institut Pasteur (“CIP” “; Institut Pasteur, 28 Rue du Docteur Roux, 75724 Paris Cedex 15, France), the Deutsche Sammlung von Mikroorganismen und Zellkulturen (“DSMZ” “; GmbH, Mascheroder Weg 1B, D-38124 Braunschweig, Germany), the IHEM Biomedical Fungi and Yeasts Collection (“IHEM” “; Scientific Institute of Public Health—Louis Pasteur, Mycology Section, Rue J. Wytsmanstraat 14, B-1050 Brussels), the Japan Collection of Microorganisms (“JCM” “; Institute of Physical and Chemical Research (RIKEN), Wako, Saitama 351-0198, Japan), the Collection of the Laboratorium voor Microbiologie en Microbiele Genetica (“LMG” “; Rijksuniversiteit, Ledeganckstraat 35, B-9000, Gent, Belgium), the MUCL (Agro)Industrial Fungi & Yeasts Collection (“MUCL,” Mycothéque de l'Universite catholique de Louvain, Place Croix du Sud 3, B-1348 Louvain-la-Neuve), the Pasteur Culture Collection of Cyanobacteria (“PCC” “; Unité de Physiologie Microbienne, Institut Pasteur, 28 rue du Docteur Roux, 75724 Paris Cedex 15, France), the All-Russian Collection of Microorganisms (“VKM” “; Russian Academy of Sciences, Institute of Biochemistry and Physiology of Microorganisms, 142292 Pushchino, Moscow Region, Russia), and the University of Texas (“UTEX” “; Department of Botany, The University of Texas at Austin, Austin, Tex. 78713-7640).

As used herein, “unicellular” refers to 1 cell that generally does not live in contact with a second cell. As used herein, “oligocellular” refers to 2 to 100 cells, including all intermediate ranges and combinations thereof, which generally live in contiguous contact with each other. Common specific types of oligocellular biological material includes 2 contacting cells (“dicellular”), three contacting cells (“tricellular”) and four contacting cells (“tetracellular”). As used herein, “multicellular” refers to 101 or more (e.g., hundreds, thousands, millions, billions, trillions), including all intermediate ranges and combinations thereof, which generally live in contiguous contact with each other. In embodiments wherein the cellular material is derived from a unicellular biological material (e.g., many microorganisms), the composition is known herein as a “unicellular-based particulate material.” In embodiments wherein the cellular material is derived from an oligocellular biological material (e.g., certain microorganisms, tissues), the composition is known herein as an “oligocellular-based particulate material,” as well as a “dicellular-based particulate material,” tricellular-based particulate material,” or “tetracellular-based particulate material,” as appropriate. In embodiments wherein the cellular material is derived from a multicellular biological material (e.g., many eukaryotic organisms such as visible plants), the composition is known herein as a “multicellular-based particulate material.” A cell-based particulate materialmay be referred to herein based upon the type of biological material from which it was derived, including taxonomic/phylogenetic classification or biochemical composition, as well as one or more processing steps used in its preparation. Examples of such lexography for a cell-based particulate materialinclude a “eurkaryotic-based particulate material,” a “prokaryotic-based particulate material,” a “plant-based particulate material,” a “microorganism-based particulate material,” a “Eubacteria-based particulate material,” an “Archaea-based particulate material,” a “fungi-based particulate material,” a “yeast-based particulate material,” a “Protista-based particulate material,” an “algae-based particulate material,” a “Chrysophyta-based particulate material,” a “Methanolacinia-based particulate material,” a “Microscilla aggregans-based particulate material,” a “bacteriophage HER-6 [44Lindberg]-based particulate material,” a “bacteria and algae-based particulate material,” a “peptidoglycan-based particulate material,” a “pellicle-based particulate material,” an “attenuated viral-based particulate material,” a “sterilized microorganism-based particulate material,” an “encapsulated Streptomyces-based particulate material,” etc.

Certain cells are capable of growth in environmental conditions typically harmful to many other types of cells (“extremophiles”), such as conditions of extreme temperature, salt or pH. The biomolecules derived from such cells makes them useful in certain embodiments for durability, activity, or other property of a biomolecular composition in a coating or other surface treatment composition that undergoes conditions similar to (e.g., the same or overlapping ranges) as those found in the cell's growth environment. For example, it is contemplated that a hyperthermophile-based biomolecular composition will find particular usefulness in coatings where high temperature thermal extremes may occur, including extremes of temperature that may occur during film formation or use of a film near a heat source. For example, a “hyperthermophile” or “thermophile” typically grows in temperatures considered herein to be a baking temperature for a coating (e.g., >40° C., often up to 120° C. or more), and some compositions may comprise biomolecules with derived from thermophiles. In other embodiments, a biomolecular composition with prolonged stability, enzymatic activity, or a combination thereof at other temperature ranges is contemplated depending upon the application. As used herein, a “psychrophile” typically grows at about −10° C. to 20° C., and a “mesophile” typically grows at about 20° C. to about 40° C., and may be used to obtain a biomolecular composition for applications in temperature ranges within or overlapping those (.e.g., ambient conditions). As used herein, an “extreme halophile” is capable of living in salt-water conditions of about 1.5 M (8.77% w/v) sodium chloride to about 2.7 M (15.78% w/v) or more sodium chloride. It is contemplated that an extreme halophile's biomolecule components will be relatively resistant to ionic-salt components of a coating or other surface treatment. As used herein, an “extreme acidophile” is capable of growing in about pH 1 to about pH6, while an “extreme alkaliphile” is capable of growing in about pH 8 to about pH 14. One or more biomolecules such as enzymes that are derived from such cells may be selected on the basis the cell's growth conditions for incorporation into the compositions described herein.

In addition to the sources described herein for biomolecules, reagents, living cells, etc., such materials and/or chemical formulas thereof may be obtained from convenient source such as a public database, a biological depository, and/or a commercial vendor. For example, various nucleotide sequences, including those that encode amino acid sequences, may be obtained at a public database, such as the Entrez Nucleotides database, which includes sequences from other databases including GenBank (e.g., CoreNucleotide), RefSeq, and PDB. Another example of a public databank for nucleotide and amino acid sequences includes the Kyoto Encyclopedia of Genes and Genomes (“KEEG”) (Kanehisa, M. et al., 2008; Kanehisa, M. et al., 2006; Kanehisa, M. and Goto, S., 2000). In another example, various amino acid sequences may be obtained at a public database, such as the Entrez databank, which includes sequences from other databases including SwissProt, PIR, PRF, PDB, Gene, GenBank, and RefSeq. Numerous nucleic acid sequences and/or encoded amino acid sequences can be obtained from such sources. In a further example, biological materials that comprise, or are capable of comprising such biomolecules (including living cells), may be obtained from a depository such as the American Type Culture Collection (“ATCC”), P.O. Box 1549 Manassas, Va. 20108, USA. In an additional example, biomolecules, chemical reagents, biological materials, and equipment may be obtained from commercial vendors such as Amersham Biosciences®, 800 Centennial Avenue, P.O. Box 1327, Piscataway, N.J. 08855-1327 USA”; BD Biosciences including Clontech®, Discovery Labware®, Immunocytometry Systems® and Pharmingen, 1020 East Meadow Circle, Palo Alto, Calif. 94303-4230 USA”; Invitrogen™, 1600 Faraday Avenue, PO Box 6482, Carlsbad, Calif. 92008 USA”; New England Biolabs®, 32 Tozer Road, Beverly, Mass. 01915-5599 USA”; Merck®, One Merck Drive, P.O. Box 100, Whitehouse Station, N.J. 08889-0100 USA”; Novagene®, 441 Charmany Dr., Madison, Wis. 53719-1234 USA”; Promega®, 2800 Woods Hollow Road, Madison Wis. 53711 USA”; Pfizer®, including Pharmacia®, 235 East 42nd Street, New York, N.Y. 10017 USA”; Quiagen®, 28159 Avenue Stanford, Valencia, Calif. 91355 USA”; Sigma-Aldrich®, including Sigma, Aldrich, Fluka, Supelco and Sigma-Aldrich Fine Chemicals, PO Box 14508, Saint Louis, Mo. 63178 USA”; Wako Pure Chemical Industries, Ltd, 1-2 Doshomachi 3-Chome, Chuo-ku, Osaka 540-8605, Japan; TCI America, 9211 N. Harborgate Street, Portland, Oreg. 97203, U.S.A.; Reactive Surfaces, Ltd, 300 West Avenue Step #1316, Austin, Tex. 78701; Stratagene®, 11011 N. Torrey Pines Road, La Jolla, Calif. 92037 USA, etc. In an further example, biomolecules, chemical reagents, biological materials, and equipment may be obtained from commercial vendors such as Amersham Biosciences®, 800 Centennial Avenue, P.O. Box 1327, Piscataway, N.J. 08855-1327 USA”; Allen Bradley, 1201 South Second Street, Milwaukee, Wis. 53204-2496, USA”; BD Biosciences®, including Clontech®, Discovery Labware®, Immunocytometry Systems® and Pharmingen, 1020 East Meadow Circle, Palo Alto, Calif. 94303-4230 USA”; Baker, Mallinckrodt Baker, Inc., 222 Red School Lane, Phillipsburg N.J. 08865, U.S.A.”; Bioexpression and Fermentation Facility, Life Sciences Building, 1057 Green Street, University of Georgia, Athens, Ga. 30602, USA”; Bioxpress Scientific, PO Box 4140, Mulgrave Victoria 3170”; Boehringer Ingelheim GmbH, Corporate Headquarters, Binger Str. 173, 55216 Ingelheim, Germany Chem Service, Inc, PO Box 599, West Chester, Pa. 19381-0599, USA”; Difco, Voigt Global Distribution Inc., P.O. Box 1130, Lawrence, Kans. 66044-8130, USA”; Fisher Scientific, 2000 Park Lane Drive, Pittsburgh, Pa. 15275, USA”; Invitrogen™, 1600 Faraday Avenue, PO Box 6482, Carlsbad, Calif. 92008 USA”; Ferro Pfanstiehl Laboratories, Inc., 1219 Glen Rock Avenue, Waukegan, Ill. 60085-0439, USA”; New England Biolabs®, 32 Tozer Road, Beverly, Mass. 01915-5599 USA”; Merck®, One Merck Drive, P.O. Box 100, Whitehouse Station, N.J. 08889-0100 USA”; Millipore Corporate Headquarters, 290 Concord Rd., Billerica, Mass. 01821, USA”; Nalgene® Labware, Nalge Nunc International, International Department, 75 Panorama Creek Drive, Rochester, N.Y. 14625. U.S.A.”; New Brunswick Scientific Co., Inc., 44 Talmadge Road, Edison, N.J. 08817 USA”; Novagene®, 441 Charmany Dr., Madison, Wis. 53719-1234 USA”; NCSRT, Inc., 1000 Goodworth Drive, Apex, N.C. 27539, USA”; Promega®, 2800 Woods Hollow Road, Madison Wis. 53711 USA”; Pfizer®, including Pharmacia®, 235 East 42nd Street, New York, N.Y. 10017 USA”; Quiagen®, 28159 Avenue Stanford, Valencia, Calif. 91355 USA”; SciLog, Inc., 8845 South Greenview Drive, Suite 4, Middleton, Wis. 53562, USA”; Sigma-Aldrich®, including Sigma, Aldrich, Fluka, Supelco, and Sigma-Aldrich Fine Chemicals, PO Box 14508, Saint Louis”; USB Corporation, 26111 Miles Road, Cleveland, Ohio 44128, USA”; Sherwin Williams Company, 101 Prospect Ave., Cleveland, Ohio, USA”; Lightnin, 135 Mt. Read Blvd., Rochester, N.Y. 14611 U.S.A.”; Amano Enzyme, USA Co., Ltd. 2150 Point Boulevard Suite 100 Elgin, Ill. 60123U.S.A.”; Novozymes North America Inc., 77 Perry Chapel Church Road, Franklinton, N.C. 27525, U.S.A.”; and WB Moore, Inc., 1049 Bushkill Drive, Easton, Pa. 18042.

In addition to those techniques specifically described herein a cell, nucleic acid sequence, amino acid sequence, and the like, may be manipulated in light of the present disclosures, using standard techniques [see, for example, In “Molecular Cloning” (Sambrook, J., and Russell, D. W., Eds.) 3rd Edition, Cold Spring Harbor, N.Y.: Cold Spring Harbor Laboratory Press, 2001”; In “Current Protocols in Molecular Biology” (Chanda, V. B. Ed.) John Wiley & Sons, 2002”; In “Current Protocols in Nucleic Acid Chemistry” (Harkins, E. W. Ed.) John Wiley & Sons, 2002”; In “Current Protocols in Protein Science” (Taylor, G. Ed.) John Wiley & Sons, 2002”; In “Current Protocols in Cell Biology” (Morgan, K. Ed.) John Wiley & Sons, 2002”; In “Current Protocols in Pharmacology” (Taylor, G. Ed.) John Wiley & Sons, 2002”; In “Current Protocols in Cytometry” (Robinson, J. P. Ed.) John Wiley & Sons, 2002”; In “Current Protocols in Immunology” (Coico, R. Ed.) John Wiley & Sons, 2002].

### B. Enzymes

Selection of a biomolecule for use depends on the property that is to be conferred to a composition. In specific embodiments, a biomolecule comprises an enzyme, as enzymatic activity is a property to be conferred to, for example, a biomolecular composition, polymeric material, coating and/or paint. As used herein, the term “enzyme” refers to a molecule that possesses the ability to accelerate a chemical reaction, and comprises one or more chemical moieties typically synthesized in living organisms, including but not limited to, an amino acid, a nucleotide, a polysaccharide or simple sugar, a lipid, or a combination thereof. Enzymes are identified by a numeric classification system [See, for example, IUBM B (1992) Enzyme Nomenclature: Recommendations (1992) of the Nomenclature Committee of the International Union of Biochemistry and Molecular Biology. (NC—ICBMB and Edwin C. Webb Eds.) Academic Press, San Diego, Calif.; Enzyme nomenclature. Recommendations 1992, 1994; Enzyme nomenclature. Recommendations 1992, 1995; Enzyme nomenclature. Recommendations 1992, 1996; Enzyme nomenclature. Recommendations 1992, 1997; and Enzyme nomenclature. Recommendations 1992, 1999].

Enzymes are typically capable of catalyzing a reaction in both directions (a “reversible reaction”), where substrate and product are converted back and forth from one to the other. The net direction of such a reversible reaction is generally dependent on the concentration of the substrate/product(s) and reaction environment, and it is contemplated that the enzyme described herein may be used in either or both reaction directions. Enzymes may function in both synthesis and degradation, catabolic and anabolic, and other types of reversible reactions. For example, an enzyme normally described as an esterase may function as an ester synthetase depending upon the concentration of the substrate(s) and/or the product(s), such as an excess of hydrolyzed esters (typically considered the product of an esterase reaction) relative to unhydrolyzed esters (typically considered the substrate of the esterase reaction). In another example, a lipase may function as a lipid synthetase depending due to a relative abundance of free fatty acids and alcohol moieties to catalyze the synthesis of fatty acid esters. Any reaction that an enzyme is capable of is contemplated, such as, for example, transesterifications, interesterifications, intraesterifications and the like being conducted by an esterase. As used herein, the term “bioactive” or “active” refers to the ability of an enzyme to accelerate a chemical reaction differentiating such activity from a like ability of a composition, and/or a method that does not comprise an enzyme to accelerate a chemical reaction. For example, a surface treatment comprising lysozyme that displays lysozyme activity would comprise an active enzyme (e.g., lysozyme EC 3.2.1.17).

In some embodiments, an enzyme comprises a proteinaceous molecule. It is contemplated that any proteinaceous molecule that functions as an enzyme, whether identical to the wild-type amino acid sequence encoded by an isolated gene, a functional equivalent of such a sequence, or a combination thereof, may be used. As used herein, a “wild-type enzyme” refers to an amino acid sequence that functions as an enzyme and is identical to the sequence encoded by an isolated gene from a natural source. As used herein, a “functional equivalent” to the wild-type enzyme generally comprises a proteinaceous molecule comprising a sequence and/or a structural analog of a wild-type enzyme's sequence and/or structure and functions as an enzyme. The functional equivalent enzyme may possess similar or the same enzymatic properties, such as catalyzing chemical reactions of the wild-type enzyme's EC classification, or may possess other enzymatic properties, such as catalyzing the chemical reactions of an enzyme that is related to the wild-type enzyme by sequence and/or structure. An enzyme encompasses its functional equivalents that catalyze the reaction catalyzed by the wild-type form of the enzyme (e.g., the reaction used for EC Classification). For example, any functional equivalent of a lipase that retains lipase activity (e.g., catalyzes the reaction: triacylglycerol+H₂O=diacylglycerol+a carboxylate), though the activity may be altered (e.g., increased reaction rates, decreased reaction rates, altered substrate preference, etc.), is encompassed by the term “lipase” (i.e., in the claims, “lipase” encompasses such functional equivalents, “human lipase” encompasses functional equivalents of a wild-type human lipase, etc.). Examples of a functional equivalent of a wild-type enzyme are described herein, and include mutations to a wild-type enzyme sequence, such as a sequence truncation, an amino acid substitution, an amino acid modification, a fusion protein, or a combination thereof, wherein the altered sequence functions as an enzyme. As used herein, the term “derived” or “obtained” refers to a biomolecule's (e.g., an enzyme) progenitor source, though the biomolecule may be wild-type or a functional equivalent of the original source biomolecule, and thus the term “derived” or “obtained” encompasses both wild-type and functional equivalents. For example, a coding sequence for a Homo sapiens enzyme may be mutated and recombinantly expressed in bacteria, and the bacteria comprising the enzyme processed into a composition for use, but the enzyme, whether isolated or comprising other bacterial cellular materials, would be “derived” from Homo sapiens. In another example, a wild-type enzyme isolated from an endogenous biological source, such as, for example, a Pseudomonas putida lipase isolated from Pseudomonas putida, would be “derived” from Pseudomonas putida.

In certain embodiments, an enzyme may comprise a simple enzyme, a complex enzyme, or a combination thereof. As known herein, a “simple enzyme” is an enzyme wherein the chemical properties of moieties found in its amino acid sequence is sufficient for producing enzymatic activity. As known herein, a “complex enzyme” is an enzyme whose catalytic activity functions when an apo-enzyme is combined with a prosthetic group, a co-factor, or a combination thereof. An “apo-enzyme” is a proteinaceous molecule and is catalytically inactive without the prosthetic group and/or co-factor. As known herein, a “prosthetic group” or “co-enzyme” is non-proteinaceous molecule that is attached to the apo-enzyme to produce a catalytically active complex enzyme. As known herein, a “holo-enzyme” is a complex enzyme that comprises an apo-enzyme and a co-enzyme. As known herein, a “co-factor” is a molecule that acts in combination with the apo-enzyme to produce a catalytically active complex enzyme. In some aspects, a prosthetic group is one or more bound metal atoms, a vitamin derivative, or a combination thereof. Examples of metal atoms that may be used as a prosthetic group and/or a co-factor include Ca, Cd, Co, Cu, Fe, Mg, Mn, Ni, Zn, or a combination thereof. Usually the metal atom is an ion, such as Ca²⁺, Cd²⁺, Co²⁺, Cu²⁺, Fe⁺², Mg²¹, Mn²⁺, Ni²¹, Zn²⁺, or a combination thereof. As known herein, a “metalloenzyme” is a complex enzyme that comprises an apo-enzyme and a prosthetic group, wherein the prosthetic group comprises a metal atom. As known herein, a “metal activated enzyme” is a complex enzyme that comprises an apo-enzyme and a co-factor, wherein the co-factor comprises a metal atom.

A chemical that binds a proteinaceous molecule is known herein as a “ligand.” As used herein, “bind” or “binding” refers to a physical contact between the proteinaceous molecule at a specific region of the proteinaceous molecule and the ligand in a reversible fashion. Examples of binding interactions include such interactions as a ligand known as an “antigen” binding an antibody, a ligand binding a receptor, and the like. A portion of the proteinaceous molecule wherein substrate binding occurs is known herein as a “binding site.” A ligand that is acted upon by the enzyme in the accelerated chemical reaction is known herein as a “substrate.” A contact between the enzyme and a substrate in a fashion suitable for the accelerated chemical reaction to proceed is known herein as “substrate binding.” A portion of the enzyme involved in the chemical interactions that contributed to the accelerated chemical reaction is known herein as an “active site.”

A chemical that slows or prevents the enzyme from conducting the accelerated chemical reaction is known herein as an “inhibitor.” A contact between the enzyme and the inhibitor in a fashion suitable for slowing or preventing the accelerated chemical reaction to proceed upon a target substrate is known herein as “inhibitor binding.” In some embodiments, inhibitor binding occurs at a binding site, an active site, or a combination thereof. In some aspects, an inhibitor's binding occurs without the inhibitor undergoing the chemical reaction. In specific aspects, the inhibitor may also be a substrate such as in the case of an inhibitor that precludes the enzyme from catalyzing the chemical reaction of a target substrate for the period of time inhibitor binding occurs at an active and/or binding site. In other aspects, an inhibitor undergoes the chemical reaction at a rate that is slower relative to a target substrate.

In some embodiments, enzymes may be described by the classification system of The International Union of Biochemistry and Molecular Biology (“IUBMB”). The IUBMB classifies enzymes by the type of reaction catalyzed and enumerates each sub-class by a designated enzyme commission number (“EC”). Based on these broad categories, an enzyme may comprise an oxidoreductase (EC 1), a transferase (EC 2), a hydrolase (EC 3), a lyase (EC 4), an isomerase (EC 5), a ligase (EC 6), or a combination thereof. Often, an enzyme may be able to catalyze multiple reactions, and thus have multiple EC classifications.

Generally, the chemical reaction catalyzed by an enzyme alters a moiety of a substrate. As used herein, a “moiety” or “group,” in the context of the field of chemistry, refers to a chemical sub-structure that is a part of a larger molecule. Examples of moiety include an acid halide, an acid anhydride, an alcohol, an aldehyde, an alkane, an alkene, an alkyl halide, an alkyne, an amide, an amine, an arene, an aryl halide, a carboxylic acid, an ester, an ether, a ketone, a nitrile, a phenol, a sulfide, a sulfonic acid, a thiol, etc.

An oxidoreductase catalyzes an oxido-reduction of a substrate, wherein the substrate is either a hydrogen donor and/or an electron donor. An oxidoreductase is generally classified by the substrate moiety that is the donor or acceptor. Examples of oxidoreductases include an oxidoreductase that acts on a donor CH—OH moiety, (EC 1.1); an donor aldehyde or a donor oxo moiety, (EC 1.2); a donor CH—CH moiety, (EC 1.3); a donor CH—NH₂ moiety, (EC 1.4); a donor CH—NH moiety, (EC 1.5); a donor nicotinamide adenine dinucleotide (“NADH”) or a donor nicotinamide adenine dinucleotide phosphate (“NADPH”), (EC 1.6); a donor nitrogenous compound, (EC 1.7); a donor sulfur moiety, (EC 1.8); a donor heme moiety, (EC 1.9); a donor diphenol or a related moiety as donor, (EC 1.10); a peroxide as an acceptor, (EC 1.11); a donor hydrogen, (EC 1.12); a single donor with incorporation of molecular oxygen (“oxygenase”), (EC 1.13); a paired donor, with incorporation or reduction of molecular oxygen, (EC 1.14); a superoxide radical as an acceptor, (EC 1.15); an oxidoreductase that oxidises a metal ion, (EC 1.16); an oxidoreductase that acts on a donor CH₂ moiety, (EC 1.17); a donor iron-sulfur protein, (EC 1.18); a donor reduced flavodoxin, (EC 1.19); a donor phosphorus or donor arsenic moiety, (EC 1.20); an oxidoreductase that acts on an X—H and an Y—H to form an X—Y bond, (EC 1.21); as well as a other oxidoreductase, (EC 1.97); or a combination thereof.

A transferase catalyzes the transfer of a moiety from a donor compound to an acceptor compound. A transferase is generally classified based on the chemical moiety transferred. Examples of transferases include a transferase that catalyzes the transfer of a one-carbon moiety, (EC 2.1); an aldehyde or a ketonic moiety, (EC 2.2); an acyl moiety, (EC 2.3); a glycosyl moiety, (EC 2.4); an alkyl or an aryl moiety other than a methyl moiety, (EC 2.5); a nitrogenous moiety, (EC 2.6); a phosphorus-containing moiety, (EC 2.7); a sulfur-containing moiety, (EC 2.8); a selenium-containing moiety, (EC 2.9); or a combination thereof.

A hydrolase catalyses the hydrolysis of a chemical bond. A hydrolase is generally classified based on the chemical bond cleaved or the moiety released or transferred by the hydrolysis reaction. Examples of hydrolases include a hydrolase that catalyzes the hydrolysis of an ester bond, (EC 3.1); a glycosyl released/transferred moiety, (EC 3.2); an ether bond, (EC 3.3); a peptide bond, (EC 3.4); a carbon-nitrogen bond, other than a peptide bond, (EC 3.5); an acid anhydride, (EC 3.6); a carbon-carbon bond, (EC 3.7); a halide bond, (EC 3.8); a phosphorus-nitrogen bond, (EC 3.9); a sulfur-nitrogen bond, (EC 3.10); a carbon-phosphorus bond, (EC 3.11); a sulfur-sulfur bond, (EC 3.12); a carbon-sulfur bond, (EC 3.13); or a combination thereof. Specific examples of a hydrolase include an esterase such as, for example, a lipase, a subtilisin (EC 3.4.21.62), a protease, such as, for example, a trypsin (EC 3.4.21.4), etc.

A lyase catalyzes the cleavage of a chemical bond by reactions other than hydrolysis or oxidation. A lyase is generally classified based on the chemical bond cleaved. Examples of lyases include a lyase that catalyzes the cleavage of a carbon-carbon bond, (EC 4.1); a carbon-oxygen bond, (EC 4.2); a carbon-nitrogen bond, (EC 4.3); a carbon-sulfur bond, (EC 4.4); a carbon-halide bond, (EC 4.5); a phosphorus-oxygen bond, (EC 4.6); an other lyase, (EC 4.99); or a combination thereof.

An isomerase catalyzes a change within one molecule. Examples of isomerases include a racemase or an epimerase, (EC 5.1); a cis-trans-isomerases, (EC 5.2); an intramolecular isomerase, (EC 5.3); an intramolecular transferase, (EC 5.4); an intramolecular lyase, (EC 5.5); an other isomerases, (EC 5.99); or a combination thereof.

A ligase catalyses the formation of a chemical bond between two substrates with the hydrolysis of a diphosphate bond of a triphosphate such as ATP. A ligase is generally classified based on the chemical bond created. Examples of lyases include a ligase that form a carbon-oxygen bond, (EC 6.1); a carbon-sulfur bond, (EC 6.2); a carbon nitrogen bond, (EC 6.3); a carbon-carbon bond, (EC 6.4); a phosphoric ester bond, (EC 6.5); or a combination thereof.

1. Antimicrobial and Antifouling Compositions

In many embodiments, a surface treatment (e.g., a coating) or a polymeric material (e.g., a plastic) comprises an antimicrobial enzyme, an antifouling enzyme, or a combination thereof. In many embodiments, an antimicrobial or an antifoulng enzyme (an enzyme that acts against a marine cell that produces fouling) acts to lyse a cell or inhibit the growth of a cell that contacts (e.g., surface contact, internal incorporation or infiltration of a material) a surface treatment or polymeric material. An antimicrobial or antifouling enzyme may act as a biocide and/or biostatic.

In some aspects, a coating comprises an antimicrobial enzyme, an antifouling enzyme, or a combination thereof. In particular aspects, the coating comprises an antimicrobial agent. In more particular aspects, the antimicrobial agent comprises an enzymatic antimicrobial agent. In certain facets, the enzymatic antimicrobial agent comprises a hydrolytic enzyme. In particular facets, the hydrolytic enzyme comprises, for example, a lysozyme. For example, a lysozyme active in a coating confers catalytic, antimicrobial activity to a coating. In alternative embodiments, a lysozyme may be used in cream or ointments, and as a pharmaceutical, primarily due to its size (14.4 kDa).

In many aspects, an enzyme catalyzes a reaction that cleaves a chemical bond in a cell wall and/or cell membrane component, allowing ease of cell lysis as the cell wall and/or membrane becomes weaker (e.g, permeabilized). While it has been previously demonstrated that ProteCoat™ (an antimicrobial peptide) is efficacious against Gram positive organisms, a combination of an antimicrobial or an antifouling enzyme (e.g., a lysozyme) demonstrates activity against cells. It is contemplated that the antimicrobial peptide compromises the membrane to allow for cell wall disruption. For example, surface treatment or polymeric material may comprise a lipolytic enzyme such as a phospholipase, cholesterol esterase, or combination thereof, that act to compromise the integrity of a cell membrane, allowing ease of access for one or more enzymes that degrade cell wall components and/or a preservative to act as well. Any combination of enzymes (e.g., antimicrobial, organophosphorous compound degrading, etc) described herein are contemplated for incorporation into a biomolecular composition, as well as incorporation into a surface treatment or a polymeric material, and may be used to confer one or more properties (e.g., one or more enzyme activities) to such compositions. Further, any such enzyme (e.g., an antimicrobial enzyme or antifouling enzyme) or enzyme combinations may be used alone or in combination with one or more other antimicrobial or antifouling agent (again in any combination), such as, for example, an antimicrobial peptide or other preservative, biocide or biostatic agent (see for example, Baldridge, G. D. et al, 2005; Hancock, R. E. W. and Scott, M. G., 2000). In particular aspects, the other antimicrobial peptide comprises ProteCoat® (Reactive Surfaces, Ltd.; also described in U.S. patent application Ser. Nos. 10/884,355; 11/368,086; and 11/865,514, each incorporated by reference). In some aspects, the other preservative, biocide, biostatic agent comprises a non-peptidic antimicrobial agent, a non-amino based antimicrobial agent, a compounded peptide antimicrobial agent, an enzyme-based antimicrobial agent, or combination thereof, described in U.S. patent application Ser. No. 11/865,514 filed Oct. 1, 2007, incorporated by reference. In some aspects, an additional antimicrobial or antifouling agent comprises a detergent (e.g., a nonionic detergent, a zwitterionic detergent, an ionic detergent), such as CHAPS (zwitterionic), a Triton X series detergent (nonionic), or SDS (ionic); a basic protein such as a protamine; a cationic polysaccharide such as chitosan; a metal ion chelator such as EDTA, all of which have particular effectiveness against lipid cellular membranes. Any combination of antimicrobial or antifouling enzyme(s) and other antimicrobial or antifouling agents may be used, though in some embodiments (e.g., Protecoat® combined a non-peptidic antimicrobial agent, a non-amino based antimicrobial agent, a compounded peptide antimicrobial agent, an enzyme-based antimicrobial agent, or combination thereof) with a improved (e.g., synergistic) effects may occur, so that the concentration of an enzyme or other antimicrobial agent may be reduced relative to use alone or in a combination with fewer antimicrobial or antifouling components. In some embodiments, the concentration of any individual antimicrobial or antifouling component comprises about 0.000000001%, 0.000000002%, 0.000000003%, 0.000000004%, 0.000000005%, 0.000000006%, 0.000000007%, 0.000000008%, 0.000000009%, 0.00000001%, 0.00000001%, 0.00000002%, 0.00000003%, 0.00000004%, 0.00000005%, 0.00000006%, 0.00000007%, 0.00000008%, 0.00000009%, 0.0000001%, 0.0000001%, 0.0000002%, 0.0000003%, 0.0000004%, 0.0000005%, 0.0000006%, 0.0000007%, 0.0000008%, 0.0000009%, 0.000001%, 0.000001%, 0.000002%, 0.000003%, 0.000004%, 0.000005%, 0.000006%, 0.000007%, 0.000008%, 0.000009%, 0.00001%, 0.00002%, 0.00003%, 0.00004%, 0.00005%, 0.00006%, 0.00007%, 0.00008%, 0.00009%, 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.11%, 1.12%, 1.13%, 1.14%, 1.15%, 1.16%, 1.17%, 1.18%, 1.19%, 1.20%, 1.21%, 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46%, 1.47%, 1.48%, 1.49%, 1.50%, 1.51%, 1.52%, 1.53%, 1.54%, 1.55%, 1.56%, 1.57%, 1.58%, 1.59%, 1.60%, 1.61%, 1.62%, 1.63%, 1.64%, 1.65%, 1.66%, 1.67%, 1.68%, 1.69%, 1.70%, 1.71%, 1.72%, 1.73%, 1.74%, 1.75%, 1.76%, 1.77%, 1.78%, 1.79%, 1.80%, 1.81%, 1.82%, 1.83%, 1.84%, 1.85%, 1.86%, 1.87%, 1.88%, 1.89%, 1.90%, 1.91%, 1.92%, 1.93%, 1.94%, 1.95%, 1.96%, 1.97%, 1.98%, 1.99%, 2.00%, 2.01%, 2.02%, 2.03%, 2.04%, 2.05%, 2.06%, 2.07%, 2.08%, 2.09%, 2.10%, 2.11%, 2.12%, 2.13%, 2.14%, 2.15%, 2.16%, 2.17%, 2.18%, 2.19%, 2.20%, 2.21%, 2.22%, 2.23%, 2.24%, 2.25%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 11%, 12%, 13%, 14%, 15%, 16%, 17%, 18%, 19%, to about 20% or more of a surface treatment or polymeric material.

A surface treatment or a polymeric material may undergo a chemical reaction or comprise a component that may partly or fully damage, inhibit, or inactivate an active biomolecule such as an enzyme. For example, a surface treatment such as a coating (e.g, a polyurethane) may cure by a chemical reaction, or a polymeric material such as an thermoset plastic may undergo a chemical cross-linking reaction. In some embodiments, the biomolecular composition (e.g., on comprising an enzyme) may be added after the bulk of a chemical reaction in a surface treatment or polymeric material has occurred. The bulk of these reactions occur during typically material preparation, are known as “body time,” “curing,” “cure time,” etc, with some residual reactions occurring after cure that is not considered significant to the potential detrimental influence on a biomolecular composition. Incorporation of the material (e.g., admixing, absorption) after part or the majority of this main cure time will serve to protect the biomolecular composition from these reactions. These cure times are typically know (e.g., described in manufacturer instructions) or readily determined by standard assays for material and/or enzyme properties. In some embodiments, the biomolecular composition is incorporated after about 1%, 2%, 3%, 5%, 6%, 7%, 8%, 9%, 10.0%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% of the cure time has passed. For example, an enzyme such as a lysozyme may be incorporated by admixing after about 80% or more of a body time as passed for a polyurethane coating. In other embodiments, a biomolecular composition may be added after 100% of the cure time has passed. For example, a biomolecular composition may be contacted with a surface treatment or polymeric material an absorbed into and/or retained on the surface of the surface treatment or polymeric material. For example, contacting a liquid component comprising a biomolecular composition that is absorbed by a surface treatment or polymeric material to incorporate the biomolecular composition into and/or on the surface of the surface treatment or polymeric material. In some embodiments, the liquid component expands the molecular pore size of the material, such as expanding a polymer matrix in a thermoplasting, allowing ease of incorporation of the biomolecular composition (e.g., a biomolecular composition comprising an enzyme) through the pores in the material. Subsequent removal of the liquid component (e.g., evaporation, heating, addition of an additional liquid component to extract the liquid component) may promote retention of the biomolecular composition by reducing the pore size, thus physically entrapping the biomolecular composition into the material. Alternatively, a biomolecular composition may undergo a chemical reaction with a component of the surface treatment or polymeric material chemically linking (e.g., a cross-linkage, a molecular tethering) the biomolecular composition to and/or within the surface treatment or polymeric material. An additional material (e.g., a catalyst, an enzyme, a reactive chemical linker, etc) may be added that undergoes or promotes a chemical reaction to chemically link a component of the surface treatment or polymeric material with a biomolecular composition. Additionally, a biomolecular composition may comprise a plurality of biomolecules or an additional material to protect the desired biomolecule from damage by chemical reactions or other components of a surface treatment or polymeric material. For example, an enzyme such as a lysozyme may comprise an additional egg white protein that protects the enzyme from loss of activity by a chemical reaction. In another example, a partly purified enzyme, cell-fragment particulate material, whole cell particulate material, encapsulated biomolecular composition (e.g., an encapsulated purified enzyme, an encapsulated cell-fragment particulate material, etc) and the like are used as they provide additional biomolecules or materials (e.g., an encapsulation material) that may protect the desired biomolecule from a chemical reaction or component of the surface treatment or polymeric material, as well as protect the desired biomolecule from damage during normal use of the surface treatment or polymeric material (e.g., environmental damage, washings, etc). In specific aspects, the desired biomolecule (e.g., an enzyme) comprises about 0.00000001%, 0.00000002%, 0.00000003%, 0.00000004%, 0.00000005%, 0.00000006%, 0.00000007%, 0.00000008%, 0.00000009%, 0.0000001%, 0.0000001%, 0.0000002%, 0.0000003%, 0.0000004%, 0.0000005%, 0.0000006%, 0.0000007%, 0.0000008%, 0.0000009%, 0.000001%, 0.000001%, 0.000002%, 0.000003%, 0.000004%, 0.000005%, 0.000006%, 0.000007%, 0.000008%, 0.000009%, 0.00001%, 0.00002%, 0.00003%, 0.00004%, 0.00005%, 0.00006%, 0.00007%, 0.00008%, 0.00009%, 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.11%, 1.12%, 1.13%, 1.14%, 1.15%, 1.16%, 1.17%, 1.18%, 1.19%, 1.20%, 1.21%, 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46%, 1.47%, 1.48%, 1.49%, 1.50%, 1.51%, 1.52%, 1.53%, 1.54%, 1.55%, 1.56%, 1.57%, 1.58%, 1.59%, 1.60%, 1.61%, 1.62%, 1.63%, 1.64%, 1.65%, 1.66%, 1.67%, 1.68%, 1.69%, 1.70%, 1.71%, 1.72%, 1.73%, 1.74%, 1.75%, 1.76%, 1.77%, 1.78%, 1.79%, 1.80%, 1.81%, 1.82%, 1.83%, 1.84%, 1.85%, 1.86%, 1.87%, 1.88%, 1.89%, 1.90%, 1.91%, 1.92%, 1.93%, 1.94%, 1.95%, 1.96%, 1.97%, 1.98%, 1.99%, 2.00%, 2.01%, 2.02%, 2.03%, 2.04%, 2.05%, 2.06%, 2.07%, 2.08%, 2.09%, 2.10%, 2.11%, 2.12%, 2.13%, 2.14%, 2.15%, 2.16%, 2.17%, 2.18%, 2.19%, 2.20%, 2.21%, 2.22%, 2.23%, 2.24%, 2.25%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.10%, 99.20%, 99.30%, 99.40%, 99.50%, 99.60%, 99.70%, 99.80%, 99.90%, 99.91%, 99.92%, 99.93%, 99.94%, 99.95%, 99.96%, 99.97%, 99.98%, 99.99%, 99.999%, 99.9999%, 99.99999%, 99.999999%, 99.9999999%, to about 100%, of the wet or dry weight of a biomolecular composition (e.g., an purified or partly purified enzyme) and/or the average content in the primary particles of a biomolecular composition, such as the primary particles of a cell-based particulate material (e.g., a cell-fragment particulate material, a whole cell particulate material, etc).

a. Antimicrobial and Antifouling Enzyme Substrates

Examples of a cell that may be targeted by such an enzyme includes a prokaryotic cell, a eukaryotic cell, or a combination thereof. In particular aspects, a prokaryotic cell, a eukaryotic cell, or combination thereof comprises a microorganism, a marine fouling organism, or a combination thereof. In certain embodiments, a target of an antimicrobial or antifouling enzyme comprises an an Archaea, a Eubacteria, a fungi, a Protista, a virus, or a combination thereof. Prokaryotic organisms are generally classified in the Kingdom Monera as Archaea (“Archaebacteria”) or Eubacteria (“bacteria”).

The Eubacteria comprises a Gram-positive Eubacteria., a Gram-negative Eubacteria., or a combination thereof A “Gram-positive Eubacteria” refers to Eubacteria comprising a cell wall that typically stains positive with Gram stain reaction (see, for example, Scherrer, R., 1984) and/or generally is not surrounded by a phospholipid bilayer (“outer cell membrane”). Gram positive bacteria generally have a cell wall composed of a thick layer of peptidoglycan overlaid by a thinner layer of techoic acid. In contrast, Gram negative bacteria have a thinner layer of peptidoglycan which is enclosed in a second lipid bilayer. A “Gram-negative Eubacteria” refers to Eubacteria comprising a cell wall that typically stains negative with Gram stain reaction and/or generally is surrounded by an outer cell membrane. A few types of “Gram-negative Eubacteria” do not stain well using a standard Gram stain procedure, however, these bacteria can be classified as a Gram-negative Eubacteria by the presence of an outer cell membrane, a morphological feature typically not present in a Gram-positive Eubacteria.

Organisms of the Kingdom Protista (“protists”) are a heterogenous set of eukaryotic unicellular, oligocellular and/or multicellular organisms that have not been classified as belonging to the other eukaryotic Kingdoms, though they typically may have features related to the Plant Kingdom (e.g., algae, which are photosynthetic), the Fungi Kingdom (e.g., Oomycota) and/or the Animal Kingdom (e.g., protozoa). Organisms of certain Protista Phyla, particularly those organisms commonly known as “algae,” comprise a cell wall, silica based shell or exoskeleton (e.g., a test, a frustule), or other durable material at the cell-external environment interface.

In many embodiments, a component of a cell wall and/or a cell membrane may both be a component of a cell-based particulate material, as well as a target of an antimicrobial or antifouling enzyme. Examples of such cell wall and/or cell membrane components comprise a peptidoglycan, a pseudopeptidoglycan, a teichoic acid, a teichuronic acid, a cellulose, a neutral polysaccharide, a chitin, a mannin, a glucan, a proteinaceous molecule, a lipid, or a combination thereof, and are described below as examples of target antimicrobial or antifouling enzyme substrates as well as possible components of a cell-based particulate material.

1. Peptidoglycans and Pseudopeptidoglycans

Eubacteria cell walls typically comprise peptidoglycan (“mucopeptide,” “murein”), as well as glycoprotein, protein, polysachamide, lipid, or a combination thereof. Peptidoglycan generally comprises alternating monomers of the amino-sugars N-acetylglucosamine and N-acetylmuramic acid. The N-acetylmuramic acid monomers often further comprise a tetra-peptide of the sequence L-alanine-D-glutamic acid-L-diamino acid-D-alanine covalently bonded to the muramic acid. The attached tetrapeptides of peptidoglycan participate in cross-linking a plurality of polymers to contribute to the cell wall structure. Depending on the species, the tetrapeptides may form the cross-linkages by direct covalent bonds, or one or more amino acids may form the cross-linking bonds between the tetrapeptides. Peptidoglycan is contemplated as being a biomolecule used in many embodiments for conferring particulate nature and durability to various cell-based particulate materials, given the general ease of growth of Eubacteria.

Archaea do not possess peptidoglycan, but many Archaea may contain pseudopeptidoglycan, which comprises N-acetyltalosaminuronic acid, instead of N-acetylmuramic in peptidoglycan. The cell wall of Archaea typically comprises pseudopeptidoglycan, as well as glycoprotein, protein, polysachamide, or a combination thereof.

2. Teichoic Acids and Teichuronic Acids

A cell wall, particularly of Gram-positive Eubacteria, may comprise up to 50% teichoic acid. Teichoic acid is an acidic polymer comprising monomers of a phosphate and glycerol; phosphate and ribitol; or N-acetylglucosamine and glycerol. A sugar (e.g., glucose) and/or an amino acid (e.g., D-alanine) is usually attached to the glycerol or ribitol of a teichoic acid. In addition to direct association with or integration into a cell wall, a teichoic acid may be associated with a phospholipid bilayer adjacent to a cell wall. Often, a teichoic acid is covalently bonded to a glycolipid of a cell membrane, and is known as a “lipoteichoic acid.” Teichic acids are common in the genera Staphylococcus, Micrococcus, Bacillus, and Lactobacillus.

A cell wall of certain species of Gram-positive Eubacteria may comprise teichuronic acid. Teichuronic acid is a polymer comprising N-acetylglucosamine and glucuronic acid or glucose and amino-mannuronic acid. However, it is thought that acidic conditions damage this cell wall component, as uronic acids such as glucuronic acid, and particularly amino-mannuronic acid, are hydrolyzed in acid. It is contemplated that exposure to acid during processing or in a surface treatment may reduce this component from a cell based particulate matter.

3. Neutral Polysaccharides

A cell wall, particularly of Gram-positive Eubacteria, may comprise a neutral polysaccharide, other than those described for a peptidoglycan, teichoic acid, cellulose, or lipopolysacharide. As used herein, a “neutral polysaccharide” is a polymer comprising a majority of neutral sugars, wherein the neutral sugar is typically a hexose or a pentose, and/or an aminosugar thereof. Examples of neutral sugars found in neutral polysaccharides include arabinose, galactose, 3-O-methyl-D-galactose, mannose, xylose, rhamnose, glucose, fructose, or a combination thereof. Examples of amino sugars found in neutral polysaccharides include glucosamine, galactosamine, or a combination thereof.

4. Proteinaceous Molecules

A cell wall may comprise a proteinaceous molecule, such as, for example, a polypeptide, a peptide, a protein, other than those described for a peptidoglycan, teichoic acid, or lipopolysacharide. As used herein, a “peptide” comprises 3 to 100 amino acids as monomers, while a “polypeptide” is a polymer comprising 101 amino acids or more as monomers. As used herein a “protein” is a proteinaceous molecule comprising a contiguous molecular sequence three amino acids or greater in length, matching the length of a biologically produced proteinaceous molecule encoded by the genome of an organism. Such proteinaceous materials may dominate the structural integrity that confers particulate material durability to a virus or a cell comprising a pellicle. Additionally, peptide linkages are common throughout peptidoglycan and pseudopeptidoglycan. However, it is contemplated that in most embodiments, a peptide or polypeptide is not the biomolecule component that dominates the overall structural integrity and/or composition of most cell walls.

5. Lipids

A cell wall may comprise a lipid, other than those described for a peptidoglycan, teichoic acid, or lipopolysacharide. Typically, a cell comprises various lipid biomolecules, which generally comprise fatty acids. It is contemplated that in embodiments wherein a processing step comprises contacting the cell with a non-aqueous solvent, most lipids will be removed from the cell and/or or cell wall. However, it is contemplated that in embodiments wherein such a processing step does not occur, the lipid components of a cell and/or cell wall remaining in the particulate matter may affect coating or other surface treatment reactions wherein lipid (e.g., fatty acid double bond) cross-linking activity contributes to film-formation. Lipids of particular relevance for such potential cross-linking reactions include those of the outer membrane, which comprise fatty acids, the cell wall, or a combination thereof.

For example, Gram-negative cells comprise a phospholipid bilayer known as the “outer cell membrane” that surrounds the cell wall. A “phospholipid bilayer” comprises two layers of phospholipid molecules, wherein the fatty acids components of each layer's phospholipids contact each other, thereby creating a hydrophobic inner region, and the head groups of each layer's phospholipids, which are generally hydrophilic, contact the external environment. Examples of a phospholipid include a glycerophospholipid, which comprises two fatty acids and one hydrophilic moiety called a “head group” covalently connected to a trihydroxyl alcohol glycerol. Non-limiting examples of a head group include choline, ethanolamine, serine, inositol, an additional glycerol or a combination thereof. Additionally, a phospholipid bilayer generally comprises a plurality of peptides and polypeptides with hydrophobic regions that are retained in the phospholipid bilayer's hydrophobic inner region. The cell wall peptidoglycan is linked to the phospholipid membrane by periplasmic space lipoprotein.

Gram-positive Eubacteria cell walls generally 0% to 2% lipid. However, certain categories of Gram-positive Eubacteria can comprise up to 50% or more lipid content as part of the cell wall. Such Eubacteria include different species of Gordonia, Mycobacterium, Nocardia, and Rhodococcus. Additionally, the lipids of such Eubacteria generally comprise a branched chain fatty acid, particularly mycolic acids (Barry, C. E. et al., Prog Lipid Res 37:143, 1998). It is though that mycolic acids are covalently bound or loosely associated with cell wall sugars. The type of Eubacteria is sometimes used to identify the carbon-backbone length of the mycolic acids. For example, an eumycolic acid is isolated from a Mycobacterium, and generally comprises 60 to 90 carbon atoms. A corynomycolic acid is isolated from a Corynobacterium, and generally comprises 22 to 36 carbons. A nocardomycoic acid is isolated from a Nocardia, and generally comprises 44 to 60 carbons. http://www.cyberlipid.org/accueil.htm; http://www.cyberlipid.org/fattyt/fatt0001.htm. A mycolic acid generally comprises a fatty acid branch (“alpha branch”) and an aldehyde (“meromycolate branch”). A mycolic acid may further comprise a carbon double bond, an epoxy ester moiety, a cyclopropane ring moiety, a keto moiety, a methoxy moiety or a combination thereof, generally located on meromycolate branch. A mycolic acid may comprise an α-mycolic acid, a methoxymycolic acid, a ketomycolic acid, an epoxymycolic acid, a wax ester or a combination thereof. A α-mycolic acid comprises a cis or trans carbon double bond and/or a cyclopropane, and may further comprise a methyl branch adjacent to such a moiety. A methoxymycolic acid comprises a methoxy moiety and a double bond or a cyclopropane. A ketomycolic acid comprises a-methyl-branched ketone. An epoxymycolic acid comprises an α-methyl-branch epoxide. A wax ester comprises an internal ester group and a carbon double bond or a cyclopropane ring.

In certain facets, a cell lipid may comprise a glycolipid, which refers to a glycan covalently attached to a lipid. Non-limiting examples of a glycolipid include a dolichyl phosphoryl glycan, a pyrophosphoryl glycan, an undecaprenyl phosphoryl glycan, a pryophosphoryl glycan, a retinyl phosphoryl glycan, a glycosphingolipid (e.g., a ceramide, a galactosphingolipid, a glucosphingolipid including a ganlioside), a glycoglycerolipid (e.g., a monogalactosyldiacylglycerol), a steroidal glycoside (e.g., ouabain, digoxin, digitonin), a glycosylated phosphoinositide (e.g., a GPI anchor, a lipophosphoglycan, a lipopeptidophosphoglycan, a glycoinositol phospholipid), or a combination thereof.

The phospholipid bilayers of Archaea are biochemically distinct from the lipids described above, as they comprise branched hydrocarbon chains attached to glycerol by ether linkages instead of fatty acids attached to glycerol by ester linkages.

6. Celluloses

A cell wall of organisms, primarily of the Kingdom Planta, comprises cellulose. Cellulose, a polysaccharide polymer composed of hundreds to thousands of glucose monomer units, is recognized as the most common organic compound on earth. It is commonly known as the structural component of the primary cell wall of green plants, as well as many forms of algae. It is typically a lnear polymer. In addition, some bacteria form a biofilm by secreting celluloe, and some Ascomycota fungal species (Ophiostomataceae) comprise cell walls made of cellulose.

7. Chitins

Fungi cells and spore wall components typically include beta-1,4-linked homopolymers of N-acetylglucosamine (“chitin”) and a glucan. Chitin is similar to cellulose, though acetylamine moiety (N-acetylglucosamine) substitutes for a hydroxyl moeity on each glucose monomer. The relative increase in hydrogen bonding between chitin polymer chains enhances the strength of a chitin-polymer matrix. The glucan is usually an alpha-glucan, such as a polymer comprising an alpha-1,3- and alpha-1,6-linkage (Griffin, 1993).

8. Agaroses

Agarose and porphyran are polysaccharide polymers, and are components of some algae (e.g., red algae).

9. Mannins and Glucans

A fungal cell wall (e.g., a yeast cell wall) may comprise oligo-mannan, helical β(1-6)-D-glucans β(1-3)-D-glucans, as well as chitin, lipids and proteins. A linkage (e.g, a β(1-4)-linkage) may occur, for example between the nonreducing ends of glucans and glycoproteins; and the reducing ends of chitin (Kollàr, R., et al., 1995; Kapteyn, J. C., et al., 1996).

b. Antimicrobial and Antifouling Enzymes

In many embodiments, an enzyme that possesses antimicrobial or antifouling activity comprises a hydrolase (EC 3). In specific embodiments, the enzyme comprises a glycosylase (EC 3.2). In more specific embodiments, the enzyme comprises a glycosidase (i.e., an enzyme that hydrolyses an O— glycosyl compound, a S-glycosyl compound, or a combination thereof) (EC 3.2.1). In particular aspects, the glycosidase acts on an O-glycosyl compound, and examples of such an enzyme includes an lysozyme, an agarase (e.g., an, a cellulose, a chitinase, or a combination thereof. In other embodiments, an antimicrobial or anti-fouling enzymes acts on a cell wall or cell membrane component, and examples of such enzymes include a lysozyme, a lysostaphin, a libiase, a lysyl endopeptidase, a mutanolysin, a cellulase, a chitinase, an α-agarase, an β-agarase, an N-acetylmuramoyl-L-alanine amidase, a lytic transglycosylase, a glucan endo-1,3-β-D-glucosidase, an endo-1,3(4)-β-glucanase, a β-lytic metalloendopeptidase, a 3-deoxy-2-octulosonidase, a peptide-N4-(N-acetyl-β-glucosaminyl)asparagine amidase, a mannosyl-glycoprotein endo-β-N-acetylglucosaminidase, a ι-carrageenase, a κ-carrageenase, a λ-carrageenase, an α-neoagaro-oligosaccharide hydrolase, an endolysin, an autolysin, a mannoprotein protease, a glucanase, a mannose, a zymolase, a lyticase. a lipolytic enzyme, or a combination thereof.

1. Lysozymes

Lysozyme (EC 3.2.1.17; CAS registry number: 9001-63-2) has been also referred to in that art as “peptidoglycan N-acetylmuramoylhydrolase,” “1,4-N-acetylmuramidase,” “globulin G,” “globulin G1,” “L-7001,” “lysozyme g,” “mucopeptide glucohydrolase,” “mucopeptide N-acetylmuramoylhydrolase,” “muramidase,” “N,O-diacetylmuramidase,” and “PR1-lysozyme.” Lysozyme catalyzes the reaction: in a peptidoglycan, hydrolyzes a (1,4)-β-linkage between N-acetylmuramic acid and a N-acetyl-D-glucosamine; in a chitodextrin (a polymer of (1,4)-β-linked N-acetyl-D-glucosamine monomers), hydrolyzes the (1,4)-β-linkage. A lysozyme demonstrates endo-N-acetylmuramidase activity, and can cleave glycan comprising linked peptides, but has little or no activity toward a glycan that lack linked peptides. In many embodiments, a lysozyme comprises a single chain protein with a MW of 14.3 kD. Lysozyme producing cells and methods for isolating a lysozyme from cellular materials and biological sources have been described [see, for example, Blade, C. C. F. et al., 1967a; Blake, C. C. F. et al., 1967b; Jolles, P., 8:227-239, 1969; Rupley, J. A., 83:245-255, 1964; Holler, H., et al., 14:2377-2385, 1975; Canfield, R. E., 238:2698-2707, 1963; Davies, R. C., et al., 178:294-305, 1969), and may be used in conjunction with the disclosures herein. A common example of a lysozyme comprises a chicken egg white lysozyme (“CEWL”). The general activity range of a CEWL lysozyme is about pH 6.0 to about 9.0, with maximal activity of lysozyme at pH 6.2 is at ionic strengths of about 0.02 M to about 0.100 M, while at pH 9.2 the maximal activity is between the ionic strengths of about 0.01 M to about 0.06 M. Lysozyme is commercially available (e.g., Sigma Aldrich).

Lysozymes comprise proteins with similar folding structures, generally divided into 9 classes. Four classes are noted for having particular effectiveness in cleaving peptidoglycan: bacteriophage T4 lysozyme, goose egg-white lysozyme, hen egg-white lysozyme, and Chaloropsis lysozyme. Two domains connected by an alpha helix form the active site, with a glutamic acid located in the N-terminal half of the protein, in the C-terminal end of an alpha-helix. Another active site residue typically is an aspartic acid. An example of a Chalaropsis lysozyme is cellosyl, differs in having an active site comprising a single, flattened ellipsoid domain with a beta/alpha fold with a long groove comprising an electronegative hole on the C-terminal face. Cellosyl is produced from Streptomyces coelicolor. An additional Chalaropsis lysozyme comprises LytC produced from Streptomyces pneumonia. Examples of autolytic lysozymes include a SF muramidase from an Enterococus faecium (“Enterococcus hirae”; ATCC 9790). Another autolytic lysozyme comprises pesticin, encoded by the pst gene on the pPCP1 plasmid from Yersinia pestis. A lysozyme has been recombinantly expressed in Aspergillus niger (Gheshlaghi et al, 2005; Archer et al. 1990; Gyamerah et al. 2002; Mainwaring et al. 1999). Examples of modifications to lysoszyme include denaturation of the lysozyme, attachement of polysaccharides or hydrophobic polypeptides to enhance effectiveness against Gram negative bacterial (Touch et al., 2003; Aminlari et al., 2005; Ibrahim et al., 1994).

In some embodiments, a lysozyme damages or destroys a bacterial cell wall, and is exemplary of the action many antimicrobial or antifouling enzymes a surface treatment or polymeric material in undermining cellular function by damaging a cell wall or membrane. A lysozyme catalyzes cleavage of a peptidoglycan's glycosidic bond between an N-acetylmuramic acid (“NAM”) and an N-acetylglucosamine (“NAG”) that often are part of a cell wall. This glycosidic crosslink braces a relatively delicate cell membrane against a cell's high osmotic pressure. As a lysozyme acts, the structural integrity of the cell wall is reduced (e.g., destroyed), and the bacteria cell bursts (“lysis”) under internal osmotic pressure. Additional mechanisms of action other than enzymatic by lysozyme may be triggered by contact with a cell may occur, such as cell membrane damage, induction of an autolysin's activity, or a combination thereof (Masschalck and Michiels, 2003). In many embodiments, a lysozyme is effective against a Gram positive bacteria since the peptidoglycan layer is relatively accessible to the enzyme, although a lysozyme is also effective against Gram negative bacteria that possess relatively less peptidoglycan in a cell wall, particularly after the outer membrane has been compromised, such as by contact with an anti-cellular membrane agent such as an antimicrobial or antifouling peptide, a detergent, a metal chelator (e.g., a metal ion chelator, EDTA) or a combination thereof.

Structural information for wild-type lysozyme and/or mutant/functional equivalent amino acid sequences for producing a lysozyme or a functional equivalent include Protein database bank entries: 102l, 103l, 104l, 107l, 108l, 109l, 110l, 111l, 112l, 113l, 114l, 115l, 116l, 118l, 119l, 120l, 122l, 123l, 125l, 126l, 127l, 128l, 129l, 130l, 131l, 132l, 133l, 134l, 135l, 137l, 138l, 139l, 140l, 141l, 142l, 143l, 144l, 145l, 146l, 147l, 148l, 149l, 150l, 151l, 152l, 153l, 154l, 155l, 156l, 157l, 158l, 159l, 160l, 161l, 162l, 163l, 164l, 165l, 166l, 167l, 168l, 169l, 170l, 171l, 1ior, 1ios, 1iot, 1ip1, 1ip2, 1ip3, 1ip4, 1ip5, 1ip6, 1ip7, 1ir7, 1ir8, 1ir9, 1ivm, 1iwt, 1iwu, 1iwv, 1iww, 1iwx, 1iwy, 1iwz, 1ix0, 1iy3, 1iy4, 1j1o, 1j1p, 1j1x, 1ja2, 1ja4, 1ja6, 1ja7, 1jef, 1jfx, 1jhl, 1jis, 1jit, 1jiy, 1jj0, 1jj1, 1jj3, 1jka, 1jkb, 1jkc, 1jkd, 1joz, 1jpo, 1jqu, 1jse, 1jsf, 1jtm, 1jtn, 1jto, 1jtp, 1jtt, 1jug, 1jwr, 1k28, 1kip, 1kiq, 1kir, 1kni, 1kqy, 1kqz, 1kr0, 1kr1, 1ks3, 1kw5, 1kw7, 1kxw, 1kxx, 1kxy, 1ky0, 1ky1, 1l00, 1l01, 1l02, 1l03, 1l04, 1l05, 1l06, 1l07, 1l08, 1l09, 1l0j, 1l0k, 1l10, 1l11, 1l12, 1l13, 1l14, 1l15, 1l16, 1l17, 1l18, 1l19, 1l20, 1l21, 1l22, 1l23, 1l24, 1l25, 1l26, 1l27, 1l28, 1l29, 1l30, 1l31, 1l32, 1l33, 1l34, 1l35, 1l36, 1l37, 1l38, 1l39, 1owz, 1oyu, 1p2c, 1p2l, 1p2r, 1p36, 1p37, 1p3n, 1p46, 1p56, 1p5c, 1p64, 1p6y, 1p7s, 1pdl, 1yil, 1ykx, 1yky, 1ykz, 1yl0, 1yl1, 1yqv, 1z55, 1zmy, 1zur, 1zv5, 1zvh, 1zvy, 1zwn, 1zyt, 200l, 201l, 205l, 206l, 207l, 208l, 209l, 210l, 211l, 212l, 213l, 214l, 215l, 216l, 217l, 2dqj, 2eiz, 2eks, 2epe, 2eql, 2f2n, 2f2q, 2f30, 2f32, 2f47, 2f4a, 2f4g, 2fbb, 2fbd, 2g4p, 2rbq, 2rbr, 2rbs, 2vbl, 2yss, 2yvb, 2z12, 2z18, 2z19, 2z2e, 2z2f, 2z6b, 3b61, 3b72, 3d3d, 3d9a, 3hfl, 3hfm, 3lhm, 3lym, 3lyo, 3lyt, 3lyz, 3lz2, 3lzm, 8lyz, 8lyz, 8lyz, 8lyz, 8lyz, 8lyz, 8lyz, 8lyz, 8lyz, 8lyz, 8lyz, 8lyz, 8lyz, 8lyz, 8lyz, 8lyz, and 8lyz. Examples of protein structure for lysozyme available in these entries include bacteriophage T4 from Escherichia coli expression; mutant T4 lysozymes (e.g., comprising an engineered metal-binding site; engineered thermostable lysozyme; 199a; 199a and m102q; cavity producing mutants; engineered salt bridge stability mutant; engineered disulfide bond mutant; g28a/i29a/g30a/c54t/c97a; 132a/133a/t34a/c54t/c97a/e108v; r14a/k16a/i17a/k19a/t21a/e22a/c54t/c97a; y24a/y25a/t26a/i27a/c54t/c97a; alternative hydrophobic core packing amino acids), sometimes from expression in Escherichia coli; mutant (e.g., i56t; asp67his; w64c and c65a; surface residue substitution; N-terminal peptide additions; i56t: t152a; t152c; t152i; t152s; t152v; v149c; v149g; v149i; v149s; synthetic lysozyme dimer; unnatural amino acid p-iodo-1-phenylalanine at position 153; engineered calcium binding site) human lysozyme, sometimes from Spodoptera frugiperda, Saccharomyces cerevisiae, or Pichia pastoris expression; Gallus gallus (chicken) lysozyme including mutant forms (e.g., d52s), including from Escherichia coli or Saccharomyces cerevisiae expression; Colinus virginianus (Bobwhite quail) lysozyme; guinea-fowl lysozyme; bacteriophage p22 lysozyme mutant (e.g., 187m) from Escherichia coli expression; Cygnus atratus (black swan goose) lysozyme; canine lysozyme from Pichia pastoris expression; Mus musculus from expressed in Escherichia coli; bacteriophage p22 mutant (e.g., 186m) from Escherichia coli expression; Streptomyces coelicolor lysozyme; turkey lysozyme; Equus caballus lysozyme; etc.

Nucleotide and protein sequences for lyozymes from various organisms are available via database such as, for example, KEGG. Examples of lyozyme and/or mutant/functional equivalent KEEG sequences for production of wild-type and/or mutant/functional equivalent nucleotide and protein sequence include: HSA-4069(LYZ); PTR-450190(LYZ); MCC-718361(LYZ); MMU-17105(Lyz2) 17110(Lyz1); RNO-25211(Lyz2); DPO-Dpse_GA11118 Dpse_GA20595; AGA-AgaP_AGAP005717 AgaP_AGAP007343 AgaP_AGAP007344 AgaP_AGAP007345 AgaP_AGAP007347 AgaP_AGAP007385; AAG-AaeL_AAEL003712 AaeL_AAEL003723 AaeL_AAEL005988 AaeL_AAEL009670 AaeL_AAEL010100 AaeL_AAEL015404; DBMO-Bmb021130; TCA-658610(LOC658610); ECC-c1436 c1562(ybcS) c3180 c4109(chiA); ECI-UTI89_C1303(ybcS1) UTI89_C1490 UTI89_C2660 UTI89_C3793(yheB) UTI89_C5112(ybcS2); ECP-ECP_{—}1160; ECV-APECO1_{—}1029 APECO1_{—}2033(ydfQ) APECO1_{—}242(ybcS2) APECO1_{—}3115(yheB) APECO1_{—}392 APECO1_{—}4196 APECO1_{—}514; ECW-EcE24377A_{—}0827; ECX-EcHS_A0304 EcHS_A0931 EcHS_A1644; ECM-EcSMS35_{—}1183; ECL-EcolC_{—}2083 EcolC_{—}2770; STY-STY2044 STY3682(nucD) STY4620(nucD2); STT-t3424(nucD) t4314(nucD); XFT-PD0996(lycV) PD1113; XFM-Xfasm12_{—}0912 Xfasm12_{—}1158; XFN-XfasM23_{—}1053 XfasM23_{—}1178; XAC-XAC1063(p13); XOP-PXO_{—}00139 PXO_{—}00141; SML-Smlt1054 Smlt1851 Smlt1944; SMT-Smal_{—}2511; VCO-VC0395_{—}1046; VHA-VIBHAR_{—}01975; PAP-PSPA7_{—}0693 PSPA7_{—}5063; PPG-PputGB1_{—}3388; PAR-Psyc_{—}1032; ABM-ABSDF0706 ABSDF1811; SON-SO_{—}0659; SDN-Sden 3256; SFR-Sfri 1671; SBL-Sbal_{—}1293 Sbal_{—}3605; SBM-Shew185_{—}2082; SBN-Sbal195_{—}0780 Sbal195_{—}2129; SDE-Sde_{—}2761; LSA-LSA1788; LSL-LSL_{—}0296 LSL_{—}0304 LSL_{—}0797 LSL_{—}0805 LSL_{—}1310; LRE-Lreu_{—}1367 Lreu_{—}1853; LRF-LAR_{—}1286; LFE-LAF_{—}1820; OOE-OEOE_{—}1199; CAC-CAC0554(lyc); CNO-NT01CX 2099; CBA-CLB_{—}2952; CBT-CLH_{—}0905 CLH_{—}2072; SEN-SACE_{—}3764 SACE_{—}7138; SYG-sync_{—}1433 sync_{—}1864; SYX-SynWH7803_{—}0779; MAR-MAE_{—}54690; ANA-alr1167; AVA-Ava_{—}4421; PMF-P9303_{—}18641; TER-Tery_{—}4180; AMR-AM1_{—}0818; CCH-Cag_{—}0702; and PPH-Ppha_{—}0875 Protein.

2. Lysostaphins

Lysostaphin (EC 3.4.24.75; CAS registry number: 9011-93-2) has been also referred to in that art as “glycyl-glycine endopeptidase.” Lysostaphin catalyzes the reaction: in a staphylococcal (e.g., S. aureus) peptidoglycan, hydrolyzes a -GlyGly- bond in a pentaglycine inter-peptide link (e.g., cleaves the polyglycine cross-links in the peptidoglycan layer of the cell wall of Staphylococcus sp.). Lysostaphin typically comprises a zinc-dependent, 25-kDa endopeptidase with an activity optimum of about pH 7.5. Lysostaphin producing cells (e.g., Staphylococcus simulans, ATCC 67080, 69764, 67079, 67076, and 67078) and methods for isolating a lysostaphin from cellular materials and biological sources have been described [see, for example, Recsei, P. A., et al., 1987; Thumm, G. and Gotz, F. 1997; Trayer, H. R., and Buckley, C. E., 1970; Browder, H. P., et al., 19, 383, 1965; Baba, T. and Schneewind, 1996], and may be used in conjunction with the disclosures herein. A lysostaphin is commercially available (e.g., Sigma Aldrich).

Structural information for wild-type lysostaphin and/or mutant/functional equivalent amino acid sequences for producing a lysostaphin or a functional equivalent include Protein database bank entries: 1QWY, 2B0P, 2B13, and 2B44. Examples of lysostaphin and/or mutant/functional equivalent KEEG sequences for production of wild-type and/or mutant/functional equivalent nucleotide and protein sequence include: HAR: HEAR2799; SAU: SA0265(lytM); SAV: SAV0276(lytM); SAW: SAHV_{—}0274(lytM); SAM: MW0252(lytM); SAR: SAR0273(lytM); SAS: SAS0252; SAC: SACOL0263(lytM); SAB: SAB0215(lytM); SAA: SAUSA300_{—}0270(lytM); SAX: USA300HOU_{—}0289(lytM); SAO: SAOUHSC_{—}00248; SAJ: SaurJH9_{—}0260; SAH: SaurJH1_{—}0267; SAE: NWMN_{—}0210(lytM); NPU: Npun_F1058 Npun_F4149 Npun_F4637 Npun_F5024 Npun_F6078; AVA: Ava_{—}0183 Ava_{—}2410 Ava_{—}3195 Ava_{—}4756 Ava_{—}4929 Ava_C0210; AMR: AM1_{—}4073 AM1_{—}5374 and AM1_B0175.

3. Libiases

Libiase comprises an enzyme obtained from Streptomyces fulvissimus (e.g, Streptomyces fulvissimus TU-6) that it typically used to promote the lysis of Gram-positive bacteria (e.g., a Lactobacillus, an Aerococcus, a Listeria, a Pneumococcus, a Streptococcus). Libiase possesses lysozyme and β-N-acetyl-D-glucosaminidase activity, with activity optimum of about pH 4, and a stability optimum of about pH 4 to about pH 8. Commercial preparations of a libiase are available (Sigma-Aldrich). Libiase producing cells and methods for isolating a libiase from cellular materials and biological sources have been described (see, for example, Niwa et al. 2005; Ohbuchi, K. et al., J. Biosci. Bioeng. 91:487, 2001), and may be used in conjunction with the disclosures herein.

4. Lysyl Endopeptidases

Lysyl endopeptidase (EC 3.4.21.50; CAS registry number: 123175-82-6) has been also referred to in that art as “Achromobacter lyticus alkaline proteinase I”; “Achromobacter proteinase I”; “achromopeptidase”; “lysyl bond specific proteinase”; and “protease I,” A lysyl endopeptidase catalyzes the peptide cleavage reaction at: Lys, including -LysPro-. In many embodiments, the lysyl endopeptidase comprises a (trypsin family) family SI peptidase. Lysyl endopeptidase producing cells and methods for isolating a lysyl endopeptidase from cellular materials and biological sources (e.g., Achromobacter lyticus-ATCC 21457; Lysobacter enzymogenes ATCC 29488, 29487, 29486, Pseudomonas aeruginosa-ATCC 29511, 21472) have been described (see, for example, Ahmed et al, 2003; Chohnan et al. 2002; Elliott, B. W. and Cohen, C. 1986; Ezaki, T. and Suzuki, S., 1982; Jekel, P. A., et al., 1983; Li et al. 1998; Masaki, T. et al. 660:51-55, 1981; Masaki, T. et al., 1981; Ohara, T. et al., 1989; Tsunasawa, S. et al., 1989), and may be used in conjunction with the disclosures herein.

An example of a lysyl endopeptidase comprises a 27 kDa “achromopeptidase” obtained from Achromobacter lyticus M497-1 may be used to promote lysis of a Gram positive bacterium typically resistant to a lysozyme. The achromopeptidase has an activity optimum of about pH 8.5 to about pH 9, and is commercially available (e.g., Sigma Aldrich; Wako Pure Chemical Industries, Ltd.). Structural information for wild-type lysyl endopeptidase and/or mutant/functional equivalent amino acid sequences for producing a lysyl endopeptidase or a functional equivalent include Protein database bank entries: 1arb and 1arc. Examples of lysyl endopeptidase and/or mutant/functional equivalent KEEG sequences for production of wild-type and/or mutant/functional equivalent nucleotide and protein sequence include: SRU: SRU_{—}1622.

5. Mutanolysins

Mutanolysin (EC 3.4.99.-) comprises a 23 kD N-acetyl muramidase obtained from Streptomyces globisporus (e.g., (ATCC 21553). Mutanolysin catalyzes the reaction: in a cell wall peptidoglycan-polysaccharide, cleavage of a N-acetylmuramyl-β(1-4)—N-acetylglucosamine bond. Examples of cells that mutanolysin acts on include Gram positive bacteria (e.g., Listeria, Lactobacillus or Lactococcus). Mutanolysin producing cells and methods for isolating a mutanolysin from cellular materials and biological sources have been described (see, for example, Assaf, N. A., and Dick, W. A., 1993; Calandra, G. B., and Cole, R. M., 1980; Fliss, I., et al., Biotechniques, 1991; Yokogawa, K., et al., 1975), and may be used in conjunction with the disclosures herein.

A mutanolysin's binding of cell wall polymers uses carboxy terminal moieties of the enzyme, so mutagenesis or truncation of those amino acids may effect binding and enzyme activity. A mutanolysin is commercially available (e.g., Sigma Aldrich).

6. Cellulases

Cellulase (EC 3.2.1.4; CAS registry number: 9012-54-8) has been also referred to in that art as “4-(1,3;1,4)-β-D-glucan 4-glucanohydrolase,” “1,4-(1,3;1,4)-β-D-glucan 4-glucanohydrolase,” “9.5 cellulase,” “alkali cellulase,” “avicelase,” “celluase A; cellulosin AP,” “celludextrinase,” “cellulase A 3,” “endo-1,4-β-D-glucanase,” “endoglucanase D,” “pancellase SS,” “β3-1,4-endoglucan hydrolase,” and “β-1,4-glucanase.” Cellulase catalyzes the reaction: in a cellulose, endohydrolysis of a (1,4)-β-D-glucosidic linkage; in a lichenin, endohydrolysis of a (1,4)-β-D-glucosidic linkage; and in a cereal β-D-glucan, endohydrolysis of a (1,4)-β-D-glucosidic linkage. In additional aspects, a cellulase often will possess the catalytic activity of: hydrolyse 1,4-linkages in β-D-glucans also containing 1,3-linkage. Cellulase producing cells and methods for isolating a cellulase from cellular materials and biological sources have been described [see, for example, Datta, P. K., et al., 1963; Myers, F. L. and Northcote, D. H., 1959; Whitaker, D. R. et al., 1963; Hatfield, R. and Nevins, D. J., 1986; Inohue, M. et al., 1999], and may be used in conjunction with the disclosures herein. A commercially available cellulase preparation (e.g., Sigma-Aldrich), often comprises an additional enzyme or retained or added during preparation, such as a hemicellulase, to aid digestion of cellulose comprising substrates.

Structural information for wild-type cellulase and/or mutant/functional equivalent amino acid sequences for producing a cellulase or a functional equivalent include Protein database bank entries: 1A39; 1A3H; 1AIW; 1CEC; 1CEM; 1CEN; 1CEO; 1CLC; 1CX1; 1DAQ; 1DAV; 1DYM; 1DYS; 1E5J; 1ECE; 1EDG; 1EG1; 1EGZ; 1F9D; 1F9O; 1FAE; 1FBO; 1FBW; 1FCE; 1G01; 1G0C; 1G87; 1G9G; 1G9J; 1GA2; 1GU3; 1GZJ; 1H0B; 1H11; 1H1N; 1H2J; 1H5V; 1H8V; 1HD5; 1HF6; 1IA6; 1IA7; 1IS9; 1J83; 1J84; 1JS4; 1K72; 1KFG; 1KS4; 1KS5; 1KS8; 1KSC; 1KSD; 1KWF; 1L1Y; 1L2A; 1L8F; 1LF1; 1NLR; 1OA2; 1OA3; 1OA4; 1O A7; 1OA9; 1OCQ; 1OJI; 1OJJ; 1OJK; 1OLQ; 1OLR; 1OVW; 1QHZ; 1QI0; 1QI2; 1TF4; 1TML; 1TVN; 1TVP; 1ULO; 1ULP; 1UT9; 1UU4; 1UU5; 1UU6; 1UWW; 1V0A; 1VJZ; 1VRX; 1W2U; 1W3K; 1W3L; 1WC2; 1WZZ; 2A39; 2A3H; 2BOD; 2BOE; 2BOF; 2BOG; 2BV9; 2BVD; 2BW8; 2BWA; 2BWC; 2CIP; 2CIT; 2CKR; 2CKS; 2DEP; 2EOP; 2E4T; 2EEX; 2EJ1; 2ENG; 2EO7; 2EQD; 2JEM; 2JEN; 2NLR; 20VW; 2QNO; 2UWA; 2UWB; 2UWC; 2V38; 2V3G; 3A3H; 3B7M; 3ENG; 3OVW; 3TF4; 4A3H; 4ENG; 4OVW; 4TF4; 5A3H; 6A3H; 7A3H; and 8A3H. Examples of cellulase and/or mutant/functional equivalent KEEG sequences for production of wild-type and/or mutant/functional equivalent nucleotide and protein sequence include: DFRU: 144551(NEWSINFRUG00000162829) 157531(NEWSINFRUG00000148215) 180346(NEWSINFRUG00000163275); DBMO: Bmb02157; CNE: CNH00790; CNB: CNBL0740; DPCH: 121193(e_gwh2.5.359.1) 129325(e_gwh2.2.646.1) 139079(e_gww2.2.208.1); LBC: LACBIDRAFT_{—}294705 LACBIDRAFT_{—}311963; DDI: DDB_{—}0215351(celA) DDB_{—}0230001; DPKN: PK11_{—}3250w; ECO: b3531(bcsZ); ECJ: JW3499(bcsZ); ECD: ECDH10B_{—}3708(bcsZ); ECE: Z4946(yhjM); ECS: ECs4411; ECC: c4343(yhjM); ECI: UTI89_C4063(yhjM); ECP: ECP_{—}3631; ECV: APECO1_{—}2917(bcsZ); ECW: EcE24377A 4019(bcsZ); ECM: EcSMS35_{—}3840(bcsZ); ECL: EcolC_{—}0186; STY: STY4183(yhjM); STT: t3900(yhjM); SPT: SPA3473(yhjM); SEK: SSPA3243; SPQ: SPAB_{—}04494; SEC: SC3551; SEH: SeHA C3933(bcsZ); SEE: SNSL254 A3889(bcsZ); SEW: SeSA_A3812(bcsZ); SEA: SeAg_B3825(bcsZ); SED: SeD_A3993(bcsZ); SEG: SG3819(bcsZ); BCN: Bcen_{—}0898; BCH: Bcen2424_{—}1380; BCM: Bcenmc03_{—}1358; BAM: Bamb_{—}1259; BAC: BamMC406_{—}1292; BMU: Bmul_{—}1925; BMJ: BMULJ_{—}01315(egl); BPS: BPSS1581(bcsZ); BPM: BURPS1710b_A0632(bcsZ); BPL: BURPS1106A_A2145; BPD: BURPS668_A2231; BTE: BTH_{—}110792; BPH: Bphy_{—}3254; BPY: Bphyt_{—}5838; PNU: Pnuc_{—}1167; BAV: BAV2628(bcsZ); AAV: Aave_{—}2102; LCH: Lcho_{—}2071 Lcho_{—}2344; AZO: azo2236(eglA); GSU: GSU2196; GME: Gmet 2294; GUR: Gura_{—}3125; GBM: Gbem 0763; PCA: Pcar 1216(sgcX); MXA: MXAN_{—}4837(celA); MTC: MT0067(celA); MRA: MRA_{—}0064(celA1) MRA_{—}1100(celA2a) MRA_{—}1101(celA2b); MTF: TBFG_{—}10061 TBFG_{—}11111; MBO: Mb0063(celA1) Mb119(celA2a) Mb1120(celA2b); MBB: BCG_{—}0093(celA1) BCG_{—}1149(celA2a) BCG_{—}1150(celA2b); MAV: MAV_{—}0326; MSM: MSMEG_{—}6752; AAS: Aasi_{—}0590; CCH: Cag_{—}0339; PLT: Plut 0993; RRS: RoseRS_{—}0349; RCA: Rcas_{—}0232; CAU: Caur_{—}1697; HAU: Haur_{—}1902; EMI: Emin_{—}0354; DRA: DR-0229; MBA: Mbar_A0214; MMA: MM_{—}0673; MBU: Mbur 0712; MEM: Memar 1505; MBN: Mboo_{—}1201; MSI: Msm 0134; MKA: MK0383; AFU: AF1795(celM); HAL: VNG1498G(celM); HSL: OE3143R; HMA: rrnAC0799(cdlM); HWA: HQ2923A(celM); NPH: NP4306A(celM); PHO: PH1171 PH1527; PAB: PAB0437 PAB0632(celB-like); PFU: PF1547; TKO: TK0781; SMR: Smar_{—}0057; HBU: Hbut 1154; PAI: PAE1385; PIS: Pisl_{—}1432; PCL: Pcal_{—}0842; PAS: Pars_{—}0452; CMA: Cmaq_{—}0206 Cmaq_{—}0950; TNE: Tneu_{—}0542; TPE: Tpen 0002 Tpen 0177; and KCR: Kcr_{—}0883 Kcr_{—}1258.

7. Chitinases

Chitinase (EC 3.2.1.14; CAS registry number: 9001-06-3) has been also referred to in that art as “(1→4)-2-acetamido-2-deoxy-β-D-glucan glycanohydrolase,” “1,4-β-poly-N-acetylglucosaminidase,” “chitodextrinase,” “poly[1,4-(N-acetyl-β-D-glucosaminide)] glycanohydrolase,” “poly-β-glucosaminidase,” and “β-1,4-poly-N-acetyl glucosamidinase.” Chitinase catalyzes the reaction: random hydrolysis of a N-acetyl-β-D-glucosaminide (1→4)-β-linkage in a chitin; and random hydrolysis of a N-acetyl-β-D-glucosaminide (1→4)-β-linkage in a chitodextrin. In additional aspects, a chitinase will possess the catalytic activity of a lysozyme. Chitinase producing cells and methods for isolating a chitinase from cellular materials and biological sources have been described [see, for example, Fischer, E. H. and Stein, E. A. Cleavage of O- and S-glycosidic bonds (survey), in Boyer, P. D., Lardy, H. and Myrbäck, K. (Eds.), The Enzymes, 2nd edn., vol. 4, pp. 301-312, 1960; Tracey, M. V. Biochem. J. 61:579-586, 1955], and may be used in conjunction with the disclosures herein. A chitinase is commercially available (e.g., Sigma Aldrich).

Structural information for wild-type chitinase and/or mutant/functional equivalent amino acid sequences for producing a chitinase or a functional equivalent include Protein database bank entries: 1CNS; 1CTN; 1D2K; 1DXJ; 1E6Z; 1ED7; 1EDQ; 1EHN; 1EIB; 1FFQ; 1FFR; 1GOI; 1GPF; 1H0G; 1H0I; 1HKI; 1HKJ; 1HKK; 1HKM; 1HVQ; 1ITX; 1K85; 1K9T; 1KFW; 1KQY; 1KQZ; 1KR0; 1KR1; 1LL4; 1LL6; 1LL7; 1LLO; 1NH6; 1O6I; 1OGB; 1OGG; 1RD6; 1UR8; 1UR9; 1W1P; 1W1T; 1W1V; 1W1Y; 1W9P; 1W9U; 1W9V; 1WAW; 1WB0; 1WNO; 1WVU; 1WVV; 1X6L; 1×6N; 2A3A; 2A3B; 2A3C; 2A3E; 2CJL; 2CWR; 2CZN; 2D49; 2 DBT; 2DKV; 2DSK; 2HVM; 21UZ; 2UY2; 2UY3; 2UY4; 2UY5; 2Z37; 2Z38; 2Z39; 3B8S; 3B9A; 3B9D; 3B9E; 3CH9; 3CHC; 3CHD; 3CHE; 3CHF; and 3CQL. Examples of chitinase and/or mutant/functional equivalent KEEG sequences for production of wild-type and/or mutant/functional equivalent nucleotide and protein sequence include: HSA: 1118(CHIT1) 27159(CHIA); PTR: 457641(CHIT1); MCC: 703284(CHIA) 703286(CHIT1); MMU: 71884(Chit1) 81600(Chia); CFA: 479904(CHIA); BTA: 282645(CHIA); DECB: 100065255(LOC100065255); MDO: 100015954(LOC100015954) 100030396(LOC100030396) 100030417(LOC100030417) 100033109(LOC100033109) 100033117(LOC100033117) 100033119(LOC100033119); OAA: 100089089(LOC100089089); GGA: 395072(CHIA); XLA: 444170(MGC80644); XTR: 448265(chitl); TCA: 641592(Chi-3) 641601(Chi-1) 652967(Cht10) 655022(Idgf4) 655122(Idgf2) 656175(LOC656175) 658736(LOC658736) 660881(Cht7) 661383(Cht4) 661428(Cht8) 661938(LOC661938); CEL: C04F6.3(cht-1); CBR: CBG14201; BMY: Bm1_{—}17035; ATH: AT3G12500(ATHCHIB) AT3G54420(ATEP3) AT5G24090; PPP: PHYPADRAFT_{—}138151 PHYPADRAFT_{—}153222 PHYPADRAFT_{—}219988 PHYPADRAFT_{—}52893 PHYPADRAFT_{—}55609; DOTA: Ot10g03210; CRE: CHLREDRAFT_{—}113089; SCE: YLR286C(CTS1); DSRD: 15784; DSMI: 15288; DSBA: 16756 26379; KLA: KLLA0C04730g; DKWA: Kwal_{—}23320; DHA: DEHA0F18073g DEHA0G06655g DEHA0G09636g; PIC: PICST_{—}31390(CHT4) PICST_{—}48142(CHT2) PICST_{—}68871(CHT3) PICST_{—}91537(CHT1); VPO: Kpol_{—}1009p7 Kpol_{—}1062p25; CGR: CAGL0A02904g CAGL0M09779g; YLI: YALI0D22396g YALI0FO4532g; NCR: NCU01393 NCU02184 NCU03026 NCU03209 NCU04500 NCU04554; PAN: PODANSgO9468 PODANSgl 191 PODANSg3325 PODANSg3488 PODANSg4492 PODANSg5997 PODANSg6135 PODANSg7650 PODANSg8762; YPG: YpAngola_A2570; YPI: YpsIP31758_{—}0611 YpsIP31758_{—}1757; YPY: YPK_{—}0693 YPK_{—}1864; YPB: YPTS_{—}3503; SSN: SSON_{—}1501(ydhO); ESA: ESA_{—}02015; KPN: KPN_{—}01993(ydhO); CKO: CKO_{—}02217; SAE: NWMN_{—}0931; LMF: LMOf2365_{—}0123(chiB); LWE: lwe0093; LLM: llmg_{—}2199(chiC); LBR: LVIS_{—}1777; CPR: CPR_{—}0949; CTH: Cthe_{—}0270; MMI: MMAR_{—}2010 MMAR_{—}2951; SGR: SGR_{—}2458; ART: Arth_{—}1229; AAU: AAur_{—}3218; TFU: Tfu_{—}0580 Tfu 0868; ACE: Acel_{—}1458 Acel_{—}1460 Acel_{—}2033; SEN: SACE_{—}2232(chiB) SACE_{—}3887(chiC) SACE_{—}5287(chiC) SACE_{—}6557 SACE_{—}6558; STP: Strop_{—}4405; SAQ: Sare_{—}3672; OTE: Oter 0638 Oter 3591; CTA: CTA_{—}0134(ydhO); CTB: CTL0382; CTL: CTLon_{—}0378; SRU: SRU_{—}2812; and HAU: Haur_{—}2750.

8. α-Agarases

α-agarase (EC 3.2.1.158; CAS no. 63952-00-1) has been also referred to in that art as “agarose 3-glycanohydrolase;” “agarase;” “agaraseA33.” α-agarase catalyzes the reaction: in agarose, endohydrolysis of a 1,3-α-L-galactosidic linkage, producing agarotetraose. Porphyran, a sulfated agarose, can also be cleaved. In additional aspects, an α-agarase obtained from a Thalassomonas sp. will possess the catalytic activity on substrates such as a neoagarohexaose (“3,6-anhydro-α-L-galactopyranosyl-(1,3)-D-galactose”) and a agarohexaose. α-agarase is enhanced by Ca²⁺. α-agarase producing cells and methods for isolating a α-agarase from cellular materials and biological sources have been described (see, for example, Ohta, Y., et al., 2005; Potin, P., et al., 1993), and may be used in conjunction with the disclosures herein.

9. β-agarases

β-agarase (EC 3.2.1.81; CAS registry number: 37288-57-6) has been also referred to in that art as “agarose 4-glycanohydrolase;” “AgaA;” “AgaB;” “agarase;” “agarose 3-glycanohydrolase;” “endo-β-agarase.” β-agarase catalyzes the reaction: in agarose, hydrolysis of a 1,4-β-D-galactosidic linkage, producing a tetramer. AgaA derived from Zobellia galactanivorans produces neoagarohexaose and neoagarotetraose, while AgaB produces neoagarobiose and neoagarotetraose. β-anomers are produced by the cleavage reaction. β-agarasealso cleaves porphyran. β-agarase producing cells and methods for isolating a β-agarase from cellular materials and biological sources have been described (see, for example, Allouch, J., et al., 2003; Duckworth, M. and Turvey, J. R. 1969; Jam, M. et al., 2005; Ohta, Y. et al., 2004a; Ohta, Y. et al., 2004b; Sugano, Y. et al., 1993), and may be used in conjunction with the disclosures herein. Structural information for wild-type β-agarase and/or mutant/functional equivalent amino acid sequences for producing a β-agarase or a functional equivalent include Protein database bank entries: 1O4Y, 1O4Z, and 1URX. Examples of β-agarase and/or mutant/functional equivalent KEEG sequences for production of wild-type and/or mutant/functional equivalent nucleotide and protein sequence include: PPF: Pput_{—}1162; PAT: Patl_{—}1904 Patl_{—}1971 Patl_{—}2341 Patl_{—}2640 Patl_{—}2642; SDE: Sde_{—}1175 Sde_{—}1176 Sde_{—}2644 Sde_{—}2650 Sde_{—}2655; RPB: RPB_{—}3029; RPD: RPD_{—}2419; RPE: RPE_{—}4620; SCO: SCO3471 (dagA); and RBA: RB3421 (agrA).

10. N-Acetylmuramoyl-L-Alanine Amidases

N-acetylmuramoyl-L-alanine amidase (EC 3.5.1.28; CAS registry number: 9013-25-6) has been also referred to in that art as “peptidoglycan amidohydrolase,” “acetylmuramoyl-alanine amidase,” “acetylmuramyl-alanine amidase,” “acetylmuramyl-L-alanine amidase,” “murein hydrolase,” “N-acetylmuramic acid L-alanine amidase,” “N-acetylmuramoyl-L-alanine amidase type I,” “N-acetylmuramoyl-L-alanine amidase type II,” “N-acetylmuramylalanine amidase,” “N-acetylmuramyl-L-alanine amidase,” and “N-acylmuramyl-L-alanine amidase” N-acetylmuramoyl-L-alanine amidase catalyzes the reaction: hydrolysis of a link between an L-amino acid residue and an N-acetylmuramoyl residue in some cell-wall glycopeptides. N-acetylmuramoyl-L-alanine amidase producing cells and methods for isolating a N-acetylmuramoyl-L-alanine amidase from cellular materials and biological sources have been described [see, for example, Ghuysen, J.-M. et al. 1969; Herbold, D. R. and Glaser, L. 1975; Ward, J. B. et al., 1982), and may be used in conjunction with the disclosures herein. Structural information for wild-type N-acetylmuramoyl-L-alanine amidase and/or mutant/functional equivalent amino acid sequences for producing a N-acetylmuramoyl-L-alanine amidase or a functional equivalent include Protein database bank entries: 1ARO, 1GVM, 1H8G, 1HCX, 1J3G, 1JWQ, 1LBA, 1X60, 1XOV, 2AR3, 2BGX, 2BH7, and 2BML. Examples of acetylmuramoyl-L-alanine amidase and/or mutant/functional equivalent KEEG sequences for production of wild-type and/or mutant/functional equivalent nucleotide and protein sequence include: HSA: 114770(PGLYRP2) 114771(PGLYRP3) 57115(PGLYRP4) 8993(PGLYRP1); PTR: 455797(PGLYRP2) 737434(PGLYRP3) 737562(PGLYRP4); MCC: 714583(LOC714583) 718287(PGLYRP2) 718480(LOC718480); MMU: 21946(Pglyrp 1) 242100(Pglyrp3) 57757(Pglyrp2); RNO: 295180(Pglyrp3b) 310611 (Pglyrp4) 499658(Pglyrp3); CFA: 610405(PGLYRP2) 612209(PGLYRP1); BTA: 282305(PGLYRP1) 510803(PGLYRP2) 532575(PGLYRP3); SSC: 396557(pPGRP-LB) 397213(PGLYRP1); GGA: 693263(PGRPL); XLA: 496035(LOC496035); ECW: EcE24377A 0941(amiD) EcE24377A 2721(amiA); ECX: EcHS_A0971(amiD) EcHS_A2572(amiA) EcHS_A2963(amiC) EcHS_A4411; SFL: SF0822 SF2488(amiA) SF2828 SF4324(amiB); SFX: S0863 S2636(amiA) S3025 S4592(amiB); SFV: SFV_{—}0855 SFV_{—}2487(amiA) SFV_{—}2895 SFV_{—}4327(amiB); SSN: SSON_{—}0853 SSON_{—}2524(amiA) SSON_{—}2974 SSON_{—}4354(amiB); SBO: SBO_{—}0800 SBO_{—}2460(amiA) SBO_{—}2707 SBO_{—}4287(amiB); PLU: pluO645(amiC) plu2790 plu4584(amiB); BUC: BU576(amiB); BAS: BUsg555(amiB); HSO: HS_{—}1082(amiB); XCV: XCV1630 XCV1812(amiC) XCV2603(amiC) XCV3978(ampD); XAC: XAC1589 XAC1780(amiC) XAC2406(amiC) XAC3860; XOO: X002368(amiC) X002445×002733(amiC) X004100; VFI: VF2326; SAE: NWMN_{—}0309 NWMN_{—}1035 NWMN_{—}1534 NWMN_{—}1773 NWMN_{—}1881; SEP: SE0750 SE1313; SPS: SPs0332; EFA: EF1293(ply-1) EF1486(ply-2); CAC: CAC0686 CAC3092(231); RCA: Rcas_{—}0212; HAU: Haur_{—}0094 Haur_{—}3648 Haur_{—}4245; EMI: Emin_{—}0232 Emin_{—}1374; RSD: TGRD_{—}681; TLE: Tlet_{—}1670; PMO: Pmob_{—}0199; and MMA: MM_{—}2290

11. Lytic Transglycosylases

A lytic transglycosylase's (“lytic murein transglycosylase,” EC 3.2.1.-) demonstrates exo-N-acetylmuramidase activity, and can cleave glycan strands comprising linked peptides or glycan strands that lack linked peptides with similar efficiency. A lysozyme and a lytic transglycosylase cleaves the β1,4-glycosidic bond between GlcNAc and MurNAc, but a lytic transglycosylase has an transglycosylation reaction producing a 1,6-anhydro ring at the MurNAc. A lytic transgrlycosylase may be inhibited by N-acetylyglucosamine thiazoline. An example of a lytic transglycosylase includes a MltB produced from Psudomonas aeruginosa. A lytic transglycosylase generally may be classified as a family 1, family 2 (e.g., MltA), family 3 (e.g., MltB) or family 4 lytic glycosylase (i.e., generally bacteriophage), based on similar amino acid sequences, particularly the conserved amino acids. Family 1 lytic transglycosylases generally are classified as 1A type (e.g, Slt70), 1B type (e.g., MltC), 1C type (e.g., EmtA), 1D type (e.g., MltD), or 1E type (e.g., YfhD). Lytic transglycosylases producing cells and methods for isolating a N-acetylmuramoyl-L-alanine amidase from cellular materials and biological sources have been described [see, for example, Holtje et al, 1975; Thunnissen et al. 1994; Scheurwater et al, 2007; Reid et al., 2004; Blackburn and Clark, 2001), and may be used in conjunction with the disclosures herein.

Crystal structures for various lytic transglycosylases include those for Neisseria gonorrhoeae MltA and E. coli MltA; E. coli Slt70; a phage X lytic transglycosylase; and E. coli Slt35 (Powell et al., 2006; van Straaten et al., 2005; van Straaten et al., 2007; van Asselt et al., 1999a; Thunnissen et al., 1994; Leung et al., 2001; van Asselt et al., 1999b). A lytic transglysosylase active site generally comprises a glutamic acid (e.g., Glu162 of Slt35; Glu478 of Slt70), with a relatively more hydrophobic active site than a goose egg white lysozyme. Another active site residue may comprise an aspartic acid (e.g., Asp308 of MltA). Structural information for wild-type lytic transglycosylase and/or mutant/functional equivalent amino acid sequences for producing a lytic transglycosylase or a functional equivalent include Protein database bank entries: 1Q2R, 1Q2S, 2PJJ, 2PIC, 1QSA, 2PNW, 1QTE, 1QUS, 1QUT, 1QDR, 1SLY, 1D0K, 1D0L, 1D0M, 3BKH, 3BKV, and 2AE0. Examples of lytic transglycosylase and/or mutant/functional equivalent KEEG sequences for production of wild-type and/or mutant/functional equivalent nucleotide and protein sequence include: ECO: b2701(mltB); ECJ: JW2671(mltB); ECE: Z4004(mltB); ECS: ECs3558; ECC: c3255(mltB); YPY: YPK_{—}1464; YEN: YE1242(mltB); SFL: SF2724(mltB); SFX: S2915(mltB); SFV: SFV_{—}2804(mltB); SSN: SSON_{—}2845(mltB); SBO: SBO_{—}2817(mltB); SBC: SbBS512_E3176(mltB); SDY: SDY_{—}2897(mltB); ECA: ECA1083(mltB); ENT: Ent638_{—}3179; ACB: AlS_{—}2316; ABM: ABSDF1210(mltB); ABY: ABAYE1161; SON: SO_{—}1166; SDN: Sden_{—}0853; SFR: Sfri 0697; SAZ: Sama_{—}2590; SBL: Sbal_{—}3277; CVI: CV_{—}1609(mltB); RSO: RSc0918(mltB); REU: Reut_A2556; REH: H16 A0808(mltB); RME: Rmet_{—}0732; BMA: BMA0417; BMV: BMASAVP1_A2561; BML: BMA10229_A0937; BMN: BMA10247_{—}0212; BXE: Bxe_A0991; BVI: Bcep1808_{—}0977; POL: Bpro_{—}3149; PNA: Pnap_{—}1216; AAV: Aave_{—}2160; AJS: Ajs_{—}2817; VEI: Veis_{—}2099; MPT: Mpe_A1242; HAR: HEAR2564(mltB); NEU: NE1033(mltB2); NET: Neut_{—}2477; YPM: YP_{—}3487(mltC); YPA: YPA 0310(mltC); YPN: YPN_{—}3152(mltC); YPS: YPTB3226(mltC); YEN: YE3445(mltC); SFL: SF2960(mltC); SFX: S3163(mltC); SFV: SFV_{—}3022(mltC); SSN: SSON_{—}3233(mltC); SBO: SBO_{—}3027(mltC); ILO: IL0198(mltC); TCX: Tcr_{—}0080; AHA: AHA_{—}3789; ASA: ASA_{—}0511(mltC); BCI: BCI_{—}0477(mltC); HHE: HH1830(mltC); WSU: WS1277; DVU: DVU1536; DVL: Dvul_{—}1595; DDE: Dde_{—}1786; LIP: LI174(mltC); ECO: b0211(mltD); ECJ: JW5018(mltD); ECE: Z0235(dniR); SBO: SBO_{—}0200(dniR); SBC: SbBS512_E0207(mltD); SDY: SDY_{—}0230(dniR); ECA: ECA3343(mltD); PLU: plu0939(mltD); SGL: SG0588; ENT: Ent638_{—}0745; CKO: CKO_{—}02972; SPE: Spro_{—}0908; VCH: VC2237; VCO: VC0395_A1829(mltD); SPC: Sputcn32_{—}1775; SSE: Ssed_{—}1988; SHE: Shewmr4_{—}2162; SHM: Shewmr7_{—}2239; SHN: Shewana3_{—}2370; SHW: Sputw3181_{—}2250; ILO: IL1698(dniR); CPS: CPS_{—}1998; NMN: NMCC_{—}1210; RSO: RSc1516(RS03787); REU: Reut_A2186; BPE: BP3214; BPA: BPP3837; BBR: BB4281; RFR: Rfer_{—}1461; DVU: DVU0041; DVL: Dvul_{—}2920; DDE: Dde_{—}3580; LIP: LI0055(mltD); FJO: Fjoh 0976; CTE: CT0979; CCH: Cag_{—}1379; CPH: Cpha266_{—}1087; PVI: Cvib_{—}0782; YPE: YP02438; YPK: y1898(mltE); YPM: YP_{—}2226(mltE1); YPA: YPA_{—}1782; YPN: YPN_{—}1892; YPS: YPTB2346; YEN: YE1901; ECI: UTI89_C5165(slt); ECP: ECP_{—}4778; SFL: SF4424(slt); SFX: S4695(slt); SFV: SFV_{—}4426(slt); SSN: SSON_{—}4542(slt); XOO: XOO0820(slt); XOM: XOO_{—}0746(XOO0746); VCH: VC0700; VCO: VC0395_A0230(slt); VVU: VV1_{—}0490; VVY: VV0706; VPA: VP0552; VFI: VF0558; VHA: VIBHAR_{—}00998; PPR: PBPRA0641; SFR: Sfri_{—}2529; SAZ: Sama_{—}1895; SBL: Sbal_{—}2273; SLO: Shew_{—}2125; SPC: Sputcn32_{—}2105; SSE: Ssed_{—}1979; SHE: Shewmr4_{—}2111; SHM: Shewmr7_{—}1863; FTL: FTL_{—}0466; FTH: FTH_{—}0463(slt); FTN: FTN_{—}0496(slt); TCX: Tcr 0924; AEH: Mlg_{—}1378; HHA: Hhal_{—}1135; ABO: ABO_{—}1587; BPS: BPSL0262; BPM: BURPS1710b_{—}0453(slt); BPL: BURPS1106A 0269; BPD: BURPS668_{—}0257; BTE: BTH_{—}10233; PNU: Pnuc_{—}1999; RFR: Rfer_{—}1088; POL: Bpro_{—}0652; PNA: Pnap_{—}0527; AAV: Aave_{—}4203; ECE: Z4130(mltA); ECS: ECs3673(mltA); ECC: c3384(mltA); ECI: UTI89_C3186(mltA); ECP: ECP_{—}2796(mltA); YPK: y3159(mltA); YPM: YP_{—}2826(mltA); YPA: YPA_{—}0496(mltA); YPN: YPN_{—}2977(mltA); YPG: YpAngola_A3225(mltA); PLU: pluO648(mltA); BUC: BU458(mltA); BAS: BUsg442(mltA); ENT: Ent638_{—}3259(mltA); CKO: CKO_{—}04178; SPE: Spro_{—}3810; HIN: HI0117(mltA); HIT: NTHI0205(mltA); CBU: CBU_{—}1111; LPN: lpg1994; LPF: lpl1970(mltA); LPP: lpp1975(mltA); BCN: Bcen_{—}2567; BCH: Bcen2424_{—}0538; BAM: Bamb_{—}0443; BMU: Bmul_{—}2856; BPS: BPSL3046; BPM: BURPS1710b_{—}3570(mltA); BPL: BURPS1106A 3578(mltA); BPD: BURPS668_{—}3551(mltA); BTE: BTH_I2905; PNU: Pnuc_{—}0151; PNE: Pnec_{—}0165; BPE: BP3268; BPA: BPP4152; BJA: blr0643; BRA: BRADO0205; MAG: amb4542; MGM: Mmc1_{—}0484; and SYP: SYNPCC7002 A2370(mltA).

12. Glucan Endo-1,3-β-D-Glucosidases

Glucan endo-1,3-β-D-glucosidase (EC 3.2.1.39; CAS registry number: 9025-37-0) has been also referred to in that art as “3-β-D-glucan glucanohydrolase,” “(1→3)-β-glucan 3-glucanohydrolase,” “1,3-β-D-glucan 3-glucanohydrolase,” “1,3-β-D-glucan glucanohydrolase,” “callase,” “endo-(1,3)-β-D-glucanase,” “endo-1,3-β-D-glucanase,” “endo-1,3-β-glucanase,” “endo-1,3-β-glucosidase,” “kitalase,” “laminaranase,” “laminarinase,” “oligo-1,3-glucosidase,” and “β-1,3-glucanase.” Glucan endo-1,3-β-D-glucosidase catalyzes the reaction: hydrolysis of (1,3)-β-D-glucosidic linkages in (1,3)-β-D-glucans. In additional aspects, a glucan endo-1,3-β-D-glucosidase often will possess the catalytic activity of hydrolyzing a laminarin, pachyman, paramylon, or combination thereof, and also have a limited hydrolysis activity against a mixed-link (1,3-1,4-)-β-D-glucan. A glucan endo-1,3-β-D-glucosidase may be useful particularly against fungal cell walls. Glucan endo-1,3-β-D-glucosidase producing cells and methods for isolating a glucan endo-1,3-β-D-glucosidase from cellular materials and biological sources have been described [see, for example, Chesters, C. G. C. and Bull, A. T., 1963; Reese, E. T. and Mandels, M., 1959; Tsuchiya, D., and Taga, M., 2001; Petit, J., et al., 10:4-5, 1994], and may be used in conjunction with the disclosures herein. An enzyme preparation comprising a glucan endo-1,3-β-D-glucosidase prepared from Rhizoctonia solani (“Kitalase”), or Trichoderma harzianum (Glucanex®), is commercially available (Sigma-Aldrich). Structural information for wild-type glucan endo-1,3-β-D-glucosidase and/or mutant/functional equivalent amino acid sequences for producing a glucan endo-1,3-β-D-glucosidase or a functional equivalent include Protein database bank entries: 1GHS, 2CYG, 2HYK, and 3DGT. Examples ofendo-1,3-β-D-glucosidase and/or mutant/functional equivalent KEEG sequences for production of wild-type and/or mutant/functional equivalent nucleotide and protein sequence include: DBMO: Bmb007310; ATH: AT3G57260(BGL2); DPOP: 769807(fgenesh4_pg.C_LG_X001297); MGR: MGG_{—}09733; TET: TTHERM_{—}00243770 TTHERM_{—}00637420 TTHERM_{—}00956460 TTHERM_{—}00956480; SFR: Sfri_{—}1319; SAZ: Sama_{—}1396; SDE: Sde_{—}3121; PIN: Ping_{—}0554; RLE: RL3815; MMR: Mmar10_{—}0247; NAR: Saro_{—}1608; SAL: Sala_{—}0919; RHA: RHA1_roO5769 RHA1 ro05771; and FJO: Fjoh_{—}2435.

13. Endo-1,3(4)-β-Glucanases

Endo-1,3(4)-β-glucanase (EC 3.2.1.6; CAS registry number: 62213-14-3) has been also referred to in that art as “3-(1→3;1→4)-β-D-glucan 3(4)-glucanohydrolase,” “1,3-(1,3;1,4)-β-D-glucan 3(4)-glucanohydrolase;” “endo-1,3-1,4-β-D-glucanase,” “endo-1,3-β-D-glucanase,” “endo-1,3-β-D-glucanase,” “endo-1,3-β-glucanase,” “endo-β-(1→3)-D-glucanase,” “endo-β-(1-3)-D-glucanase,” “endo-β-1,3(4)-glucanase,” “endo-β-1,3-1,4-glucanase,” “endo-β-1,3-glucanase IV,” “laminaranase,” “laminarinase,” “β-1,3-1,4-glucanase,” and “β-1,3-glucanase.” Endo-1,3(4)-β-glucanase catalyzes the reaction: endohydrolysis of (1,3)-linkages in β-D-glucans and/or (1,4)-linkages in β-D-glucans, wherein the hydrolyzed link's glucose residue is substituted at a C-3 of the reducing moiety that is part of the substrate chemical linkage. Endo-1,3(4)-β-glucanase producing cells and methods for isolating an endo-1,3(4)-β-glucanase from cellular materials and biological sources have been described [see, for example, Barras, D. R. and Stone, B. A., 1969a; Barras, D. R. and Stone, B. A., 1969b; Cunningham, L. W. and Manners, D. J., 1961; Reese, E. T. and Mandels, M., 1959; Sova, V. V., Elyakova, L. A. and Vaskovsky, V. E., 1970], and may be used in conjunction with the disclosures herein. Structural information for wild-type endo-1,3(4)-β-glucanase and/or mutant/functional equivalent amino acid sequences for producing an endo-1,3(4)-β-glucanase or a functional equivalent include Protein database bank entries: 1UP4, 1UP6, 1UP7, and 2CL2. Examples of endo-1,3(4)-β-glucanase and/or mutant/functional equivalent KEEG sequences for production of wild-type and/or mutant/functional equivalent nucleotide and protein sequence include: NCR: NCU04431 NCU07076; PAN: PODANSg699 PODANSg9033; FGR: FG04768.1 FG06119.1 FG08757.1; AFM: AFUA_{—}1G04260AFUA_{—}1G05290AFUA_{—}3G03080AFUA_{—}4G13360; AFUA_{—}5G02280 AFUA_{—}5G13990 AFUA_{—}5G14030 AFUA_{—}6G14540; ANG: An01g03090; DPCH: 10833(fgenesh1_pm.C_scaffold_{—}14000004) 123909(e_gwh2.6.417.1); LBC: LACBIDRAFT_{—}174636 LACBIDRAFT_{—}191735 LACBIDRAFT_{—}250640; LACBIDRAFT_{—}255995; PFA: PFL0285w; PFH: PFHG_{—}03986; PYO: PY01776; DPKN: PK12_{—}0440w; BCL: ABC2683 ABC2776; OIH: OB2143; CBE: Cbei_{—}2710; HWA: HQ2923A(celM); and NPH: NP4306A(celM).

14. β-Lytic Metalloendopeptidases

β3-lytic metalloendopeptidase (EC 3.4.24.32; CAS no. 37288-92-9) has been also referred to in that art as “achromopeptidase component;” “Myxobacter β-lytic proteinase;” “Myxobacter495 β-lytic proteinase; “Myxobacterium sorangium β-lytic proteinase;” “β-lytic metalloproteinase;” or “β-lytic protease.” β-lytic metalloendopeptidase catalyzes the reaction: N-acetylmuramoylAla cleavage, as well as insulin B chain cleavage. β-lytic metalloendopeptidase may be used, for example, against bacterial cell walls. β-lytic metalloendopeptidase producing cells and methods for isolating a β-lytic metalloendopeptidase from cellular materials and biological sources (e.g., Achromobacter lyticus; Lysobacter enzymogenes) have been described [see, for example, Whitaker, D. R. et al., 1965; Whitaker, D. R. and Roy, C., 1967; Li, S. L. et al., 1990; Altmann, F. et al., 1986; Plummer, T. H., Jr. and Tarentino, A. L., 1981; Takahashi, N., 1977; Takahashi, N. and Nishibe, H., 1978; Tarentino, A. L. et al., 1985.], and may be used in conjunction with the disclosures herein.

15. 3-Deoxy-2-Octulosonidases

3-deoxy-2-octulosonidase (EC 3.2.1.124; CAS no. 103171-48-8) has been also referred to in that art as “capsular-polysaccharide 3-deoxy-D-manno-2-octulosonohydrolase;” “2-keto-3-deoxyoctonate hydrolase;” “octulofuranosylono hydrolase;” or “octulopyranosylonohydrolase; or “octulosylono hydrolase.” 3-deoxy-2-octulosonidase catalyzes the reaction: endohydrolysis of the β-ketopyranosidic linkages of 3-deoxy-D-manno-2-octulosonate in capsular polysaccharides. 3-deoxy-2-octulosonidase acts on polysaccharises of bacterial (e.g., Escherichia coli) cell walls. 3-deoxy-2-octulosonidase producing cells and methods for isolating a 3-deoxy-2-octulosonidase from cellular materials and biological sources have been described [see, for example, Altmann, F. et al., 1986], and may be used in conjunction with the disclosures herein.

16. Peptide-N4-(N-acetyl-β-Glucosaminyl)asparagine Amidases

Peptide-N4-(N-acetyl-β-glucosaminyl)asparagine amidase (EC 3.5.1.52; CAS no. 83534-39-8) has been also referred to in that art as “N-linked-glycopeptide-(N-acetyl-β-D-glucosaminyl)-L-asparagine amidohydrolase;” “glycopeptidase;” “glycopeptide N-glycosidase;” “Jack-bean glycopeptidase;” “N-glycanase;” “N-oligosaccharide glycopeptidase;” “PNGase A;” or “PNGase F.” Peptide-N4-(N-acetyl-β-glucosaminyl)asparagine amidase catalyzes the reaction: hydrolysis of an N4-(acetyl-β-D-glucosaminyl)asparagine residue. The reaction may promote the glycosylation of the glyglucosamine residue, and preduce a peptide comprising an aspartate and a substituted N-acetyl-β-D-glucosaminylamine. Peptide-N4-(N-acetyl-β-glucosaminyl)asparagine amidase does not substantively act on (GlcNAc)Asn, as 3 or more amino acids in the substrate allows the reaction to proceed. Peptide-N4-(N-acetyl-β-glucosaminyl)asparagine amidase producing cells and methods for isolating a eptide-N4-(N-acetyl-β-glucosaminyl)asparagine amidase from cellular materials and biological sources have been described [see, for example, Plummer, T. H., Jr. and Tarentino, A. L., 1981; Takahashi, N. and Nishibe, H., 1978; Takahashi, N., 1977; Tarentino, A. L. et al., 1985], and may be used in conjunction with the disclosures herein. Structural information for wild-type peptide-N4-(N-acetyl-β-glucosaminyl)asparagine amidase and/or mutant/functional equivalent amino acid sequences for producing a peptide-N4-(N-acetyl-β-glucosaminyl)asparagine amidase or a functional equivalent include Protein database bank entries: 1PGS, 1PNF, 1PNG, 1X3W, 1X3Z, 2D5U, 2F4M, 2F40, 2G9F, 2G9G, 2HPJ, 2HPL, and 2I74. Examples of peptide-N4-(N-acetyl-β-glucosaminyl)asparagine amidase and/or mutant/functional equivalent KEEG sequences for production of wild-type and/or mutant/functional equivalent nucleotide and protein sequence include: HSA: 55768(NGLY1); PTR: 460233(NGLY1); MCC: 700842(LOC700842); DECB: 100059456(LOC100059456); OAA: 100075786(LOC100075786); GGA: 420655(NGLY1); DRE: 553627(zgc:110561); DFRU: 139051(NEWSINFRUG00000131342); DTNI: 33706; DOLA: 10847(ENSORLG00000008647); DCIN: 289359(estExt_fgenesh3_pg.C_chr_{—}05q0441); DME: Dmel_CG7865(PNGase); DPO: Dpse_GA20643; AGA: AgaP_AGAP007390; AAG: AaeL_AAEL014507; DAME: 9653(ENSAPMG00000005556); DBMO: Bmb025391; TCA: 664307(LOC664307); BMY: Bm1_{—}49720; ATH: AT5G49570(ATPNG1); DPOP: 241215(gw1.XIII.1464.1); DVVI: GSVIVP00031149001(GSVIVT00031149001); OSA: 4343301(Os07g0497-400); PPP: PHYPADRAFT_{—}151482; OLU: OSTLU 5312; DOTA: Ot14g02360; CRE: CHLREDRAFT_{—}146964; DHA: DEHA0E22572g; VPO: Kpol_{—}1074p3; CGR: CAGLOH05753g; YLI: YALI0C23562g; NCR: NCU00651; FGR: FG01650.1; MBR: MONBRDRAFT_{—}8805; and DTPS: 35410(e_gw1.7.250.1).

17. Mannosyl-Glycoprotein Endo-β-N-Acetylglucosaminidases

Mannosyl-glycoprotein endo-β-N-acetylglucosaminidase (EC EC 3.2.1.96; CAS no. 37278-88-9) has been also referred to in that art as “glycopeptide-D-mannosyl-N4-(N-acetyl-D-glucosaminyl)-2-asparagine 1,4-N-acetyl-β-glucosaminohydrolase;” “di-N-acetylchitobiosyl β-N-acetylglucosaminidase;” “endoglycosidase S;” “endo-N-acetyl-β-D-glucosaminidase;” “endo-N-acetyl-β-glucosaminidase;” “endo-β-(14)-N-acetylglucosaminidase;” “endo-β-acetylglucosaminidase;” “endo-β-N-acetylglucosaminidase D;” “endo-β-N-acetylglucosaminidase F;” “endo-β-N-acetylglucosaminidase H;” “endo-β-N-acetylglucosaminidase L; “endo-β-N-acetylglucosaminidase;” “mannosyl-glycoprotein 1,4-N-acetamidodeoxy-β-D-glycohydrolase;” “mannosyl-glycoprotein endo-β-N-acetylglucosamidase;” or “N,N′-diacetylchitobiosyl β-N-acetylglucosaminidase.” Mannosyl-glycoprotein endo-β-N-acetylglucosaminidase catalyzes the reaction: N,N′-diacetylchitobiosyl unit endohydrolysis in high-mannose glycoproteins and glycopeptides comprising -[Man(GlcNAc)₂]Asn- structure, wherein the intact oligoshaccharid is released and a N-acetyl-D-glucosamine residue is still attached to the protein. Mannosyl-glycoprotein endo-β-N-acetylglucosaminidase producing cells and methods for isolating a mannosyl-glycoprotein endo-β-N-acetylglucosaminidase from cellular materials and biological sources have been described [see, for example, Chien, S., et al., 1977; Koide, N. and Muramatsu, T., 1974; Pierce, R. J. et al., 1979; Pierce, R. J. et al., 1980; Tai, T. et al., 1975; Tarentino, A. L., et al., 1974.], and may be used in conjunction with the disclosures herein. Structural information for wild-type mannosyl-glycoprotein endo-β-N-acetylglucosaminidase and/or mutant/functional equivalent amino acid sequences for producing a mannosyl-glycoprotein endo-β-N-acetylglucosaminidase or a functional equivalent include Protein database bank entries: 1C3F, 1C8X, 1C₈Y, 1C90, 1C91, 1C92, 1C93, 1EDT, 1EOK, 1EOM, and 2EBN. Examples of mannosyl-glycoprotein endo-β-N-acetylglucosaminidase and/or mutant/functional equivalent KEEG sequences for production of wild-type and/or mutant/functional equivalent nucleotide and protein sequence include: HSA: 64772(FLJ21865); OAA: 100089364(LOC100089364); DCIN: 254322(gw1.55.22.1); DAME: 24424(ENSAPMGOOOOOO15707) 33583(ENSAPMGOOOOOO15707); DBMO: BmbO29819; TCA: 658146(LOC658146); BMY: Bm1_{—}17595; DHA: DEHAO020174g; PIC: PICST_{—}32069(HEX1); MBR: MONBRDRAFT_{—}34057; TBR: Tb09.160.2050; BCL: ABC3097; LSP: Bsph 1040; SAU: SA0905(atl); SAV: SAV1052; SAW: SAHV_{—}1045; SAM: MW0936(atl); SAR: SAR1026(atl); SAS: SAS0988; SAC: SACOL1062(atl); SHA: SH1911(atl); SSP: SSP1741; LLM: llmg_{—}1087(acmC) llmg_{—}2165(acmB); SPZ: M5005_Spy_{—}1540(endoS); SPH: MGAS10270_Spy1607(endoS); SPI: MGAS10750_Spy1599(endoS); SPJ: MGAS2096_Spy1565(endoS); SPK: MGAS9429_Spy1544(endoS); SPF: SpyM50309; SPA: M6_Spy1530; SPB: M28_Spy1527(endoS); LBR: LVIS_{—}1883; OOE: OEOE_{—}0144; CNO: NT01CX_{—}0726; CBA: CLB_{—}3142; BLJ: BLD_{—}0197; and CHU: CHU_{—}1472(flgJ).

18. ι-Carrageenases

ι-carrageenase (EC 3.2.1.157) has been also referred to in that art as “ι-carrageenan 4-β-D-glycanohydrolase (configuration-inverting).” ι-carrageenase catalyzes the reaction: in ι-carrageenan, endohydrolysis of a 1,4-β-D-linkage between 3,6-anhydro-D-galactose-2-sulfate and D-galactose 4-sulfate. ι-carrageenase producing cells and methods for isolating an 1-carrageenase from cellular materials and biological sources have been described [see, for example, Barbeyron, T. et al., 2000; Michel, G. et al., 2001; Michel, G. et al., 2003], and may be used in conjunction with the disclosures herein. Structural information for wild-type ι-carrageenase and/or mutant/functional equivalent amino acid sequences for producing a ι-carrageenase or a functional equivalent include Protein database bank entries: 1H80 and 1KTW.

19. κ-Carrageenases

κ-carrageenase (EC 3.2.1.83; CAS no. 37288-59-8) has been also referred to in that art as “κ-carrageenan 4-β-D-glycanohydrolase;” “κ-carrageenan 4-β-D-glycanohydrolase (configuration-retaining).” κ-carrageenase catalyzes the reaction: in κ-carrageenans, endohydrolysis of a 1,4-β-D-linkage between a 3,6-anhydro-D-galactose and a D-galactose 4-sulfate. κ-carrageenase often acts against algae (e.g, red algae). κ-carrageenase producing cells and methods for isolating a κ-carrageenase from cellular materials and biological sources have been described [see, for example, Weigl, J. and Yashe, W., 1966; Potin, P. et al., 1991; Potin, P. et al., 1995; Michel, G. et al., 1999; Michel, G., et al., 2001.], and may be used in conjunction with the disclosures herein. Structural information for wild-type κ-carrageenase and/or mutant/functional equivalent amino acid sequences for producing a κ-carrageenase or a functional equivalent include Protein database bank entries: 1DYP. Examples of κ-carrageenase and/or mutant/functional equivalent KEEG sequences for production of wild-type and/or mutant/functional equivalent nucleotide and protein sequence include: RBA: RB2702.

20. λ-Carrageenases

λ-carrageenase (EC 3.2.1.162) has been also referred to in that art as “endo-(1→4)-β-carrageenose 2,6,2′-trisulfate-hydrolase;” or “endo-β-1,4-carrageenose 2,6,2′-trisulfate-hydrolase.” λ-carrageenase catalyzes the reaction: in λ-carrageenan, endohydrolysis of a (1,4)-β-linkage, producing a α-D-Galp2,6S2-(1,3)-β-D-Galp2S-(1,4)-α-D-Galp-2,6S2-(1,3)-D-Galp2S tetrasaccharide. λ-carrageenase producing cells and methods for isolating a λ-carrageenase from cellular materials (e.g, Pseudoalteromonas sp) and biological sources have been described [see, for example, Ohta, Y. and Hatada, 2006], and may be used in conjunction with the disclosures herein.

21. α-Neoagaro-Oligosaccharide Hydrolases

α-neoagaro-oligosaccharide hydrolase (EC 3.2.1.159) has been also referred to in that art as “α-neoagaro-oligosaccharide 3-glycohydrolase;” “α-neoagarooligosaccharide hydrolase;” or “α-NAOS hydrolase.” α-neoagaro-oligosaccharide hydrolase catalyzes the reaction: hydrolysis of a 1,3-α-L-galactosidic linkage in a neoagaro-oligosaccharide, wherein the substrate is a pentamer or smaller, producing D-galactose and 3,6-anhydro-L-galactose. α-neoagaro-oligosaccharide hydrolase producing cells and methods for isolating a NAME from cellular materials and biological sources have been described [see, for example, Sugano, Y., et al. 1994], and may be used in conjunction with the disclosures herein.

22. Additional Antimicrobial or Antifouling Enzymes

An endolysin may be used particularly for a Gram positive bacteria, particularly one that may be resistant to a lysozyme. An endolysin is a phage encoded enzyme that foster release of new phage by destruction of a cell wall. An endolysin may be an N-acetylmuramidase, an N-acetylglucosaminidae, an emdopeptidase, or an amidase. Endolysin are typically translocated by phage encoded holin protein disrupting the cytosolic membrane (Wang et al., 2000). LysK lysine from phage k and Listeria monocytogenes bacteriophage-lysin have been recombinately expressed in Lactoccus lactus and/or E. coli (Loessner et al. 1995; Gaeng et al. 2000; O'Flaherty et al. 2005). An autolysin such as, for example, from Staphylococcus aureus, Bacillus subtilis, or Streptococcus pneumonia, may also be used as an antimicrobial or antifouling enzyme (Smith et al, 2000; Lopez et al. 2000; Foster et al. 1995).

A protease may be used to cleave the mannoprotein outer cell wall layer, particularly for a fungi such as yeast. An glucanase such as, for example, a beta(1->6) glucanase, a glucan endo-1,3-β-D-glucosidase, or an endo-1,3(4)-β-glucanase can then can more easily cleave glucan from the inner cell wall layers. Combinations of a protease and a glucanase may be used to produce improved lytic activity. A reducing agent, such as a dithiothreitol of beta-mercaptoethanol may aid in allowing enzyme contact with the inner cell wall by breaking disulfide linkages, such as between a cell wall protein and a mannose. Mannase and/or chitinase may also aid cell wall component cleavage. A proteinase, a pectinase, an amylase, or a combination thereof may also be used. Examples of enzymes that degrade fungal cell walls include those produced by an Arthrobacter sp., Celluloseimicrobium cellulans (“Oerskovia xanthineolytica LL G109”) (DSM 10297), Cellulosimicrobium cellulans (“Arthobacter lueus 73/14”) (ATCC 21606), Cellulosimicrobium cellulans TK-1, Rarobacter faecitabidus, Rhizoctonia sp., or a combination thereof. An Arthrobacter sp. produces a protease with functional optimums of about pH 11 and about 55° C. (Adamitsch et al., 2003). A Celluloseimicrobium cellulans (ATCC 21606) produces a protease and a glucanase (“lyticase”) with functional optimums of about pH 10 and about pH 8.0, respectively (Scott and Schekman, 1980; Shen et al., 1991). A Celluloseimicrobium cellulans (DSM 10297) produces a protease with functional optimums of about pH 9.5 to about pH 10, and a glucanase with a functional optimum of about pH 8.0 and about 40° C. (Salazar et al. 2001; Ventom and Asenjo, 1990). A Rarobacter faecitabidus produces a protease effective against cell wall a component (Shimoi et al, 1992). A Rarobacter sp. produces a glucanase with a functional optimum of about pH 6 to about pH 7, and 40° C. (Kobayashi et al. 1981). In specific aspects, commercially available enzyme preparations such as a zymolase or lyticase (Sigma-Aldrich), generally comprising a 1,3-glucanase and other enzymes, may be used.

In other embodiments, an antimicrobial or antifouling enzyme is combined with another enzyme (i.e., an additional enzyme), for the purpose to confer an additional catalytic or binding property to a surface treatment, or polymeric composition. Examples of an additional enzyme comprise a lipolytic enzyme, though some lipolytic enzymes may have antimicrobial or antifouling activity; a phosphoric triester hydrolase; a sulfuric ester hydrolase; a peptidase, some of which may have an antimicrobial or antifouling activity; a peroxidase, or a combination thereof. Alternatively, in several embodiments, an additional enzyme may be used with little or no an antimicrobial or antifouling enzyme. For example, a surface treatment or a polymeric composition may comprise a combination of active additional enzymes with little or no active antimarine or active an antifouling enzyme present.

2. Additional Enzymes: Lipolytic Enzymes

An additional enzyme for use comprises a lipolytic enzyme, which as used herein comprises an enzyme that catalyzes a reaction or series of reactions on a lipid substrate that produces one or more products that are more aqueous soluble, absorb easier into a coating or film, or an effective combination thereof, than the lipid substrate. In some embodiments, the enzyme catalyzes hydrolysis of a fatty acid bond, usually an ester bond. In other embodiments, the products produced are a free fatty acid, an alcohol moiety comprising product, or a combination thereof. In specific embodiments, at least one produce is soluble in an aqueous media such as water comprising detergent.

As used herein, a “lipid” is a hydrophobic or amphipathic organic molecule extractable with a non-aqueous solvent, such as, for example, a triglyceride, a diglyceride, a monoglyceride, a phospholipid, a glycolipid (e.g., galactolipid), a steroid (e.g., cholesterol), a wax, a fat-soluble vitamin (e.g., vitamin A, D, E, K), a petroleum based material, such as, for example, a hydrocarbon composition such as gasoline, a crude petroleum oil, grease greases, etc., or an combination thereof. A lipid may comprise a combination (mixture) of lipids, such as a grease that comprises both a fatty acid based lipid and a petroleum based lipid. Some lipids are apolar (e.g., a hydrocarbons, a carotene), others are polar (e.g., triacylglycerol, a retinol, a wax, a sterol), and some polar lipids may have partial solubility in water (e.g., a lysophospholipid). Because of the prevalence of these types of lipids in activities such as, for example, restaurant food preparation and counterpart use in household applications, a coating and/or surface treatment will be formulated to comprise one or more lipolytic enzymes to promote lipid removal from surfaces contaminated with a lipid in these environments.

Lipolytic enzymes have been identified in cells across the phylogenetic categories, and purified for analysis or use in commercial applications (Brockerhoff, Hans and Jensen, Robert G. “Lipolytic Enzymes,” 1974). Further, numerous nucleotide sequences for lipolytic enzymes have been isolated, the encoded protein sequence determined, and in many cases the nucleotide sequences recombinantly expressed for high level production of lipolytic enzymes (e.g., lipases), particularly for isolation, purification and subsequent use in industrial/commercial applications [“Lipases their Structure, Biochemistry and Application” (Paul Woolley and Steffen B. Peterson, Eds.) 1994].

Many lipolytic enzymes are classified as an alpha/beta fold hydrolase (“alpha/beta hydrolase”), due to a structural configuration generally comprising an 8 member beta pleated sheet, where many sheets are parallel, with several alpha helices on both sides of the sheet. A lipolytic enzyme's amino acid sequence commonly has Ser, Glu/Asp, His active site residues (e.g., Ser152, Asp176, and His263 by human pancreatic numbering). The Ser is comprised in a GXSXG substrate binding consensus sequence for many types of lipolytic enzymes, with a GGYSQGXA sequence being present in a cutinase. The active site serine is generally at a turn between a beta-strand and an alpha helix, and these lipolytic enzymes are classified as serine esterases. A substitution at the 1^{st} position Gly (e.g., Thr) has been identified in some lipolytic enzymes. Often a Pro residue is found at the residues 1 and 4 down from the Asp, and the His is typically within a CXHXR sequence. A lipolytic enzyme will generally have an alpha helix flap (a.k.a. “lid”) region (around amino acid residues 240-260 by human pancreatic lipase numbering) covering the active site, with a conserved tryptophan in this region in proximity of the active site serine in many lipolytic enzymes [In “Advances in Protein Chemistry, Volume 45 Lipoproteins, Apolipoproteins, and Lipases.” (Anfinsen, C. B., Edsall, J. T., Richards, Frederic, R. M., Eisenberg, D. S., and Schumaker, V. N. Eds.) Academic Press, Inc., San Diego, Calif., pp. 1-152, 1994; “Lipases their Structure, Biochemistry and Application” (Paul Woolley and Steffen B. Peterson, Eds.), pp. 1-243-270, 337-354, 1994.]. It is contemplated that all such alpha/beta hydrolases, particularly those possessing lipolytic activity, may be used.

A lipolytic alpha/beta hydrolase's catalysis is usually dependent upon or stimulated by interfacial activation, which is the contact of a lipase with an interface where two layers of materials with differing hydrophobic/hydrophilic character meet, such as a water/oil interface of a micelle or emulsion, an air/water interface, or a solid carrier/organic solvent interface of an immobilized lipase. Interfacial activation is thought to result from lipid substrate forming an ordered confirmation in a localized hydrophobic environment, so that the substrate is more easily bound by a lipolytic enzyme than a lipid substrate's conformation in a hydrophilic environment. A conformational change in the flap region due to contact with the interface allows substrate binding in many alpha/beta hydrolases. Cutinase is lipolytic alpha/beta hydrolase that is not substantially enhanced by interfacial activation. The apparent difference in a cutinase is a lack of a lid, and the ability to bury the aliphatic FA chains in the active site cleft without the charge effects of an interface prompting a conformational change in the enzyme [In “Engineering of/with Lipases” (F. Xavier Malcata., Ed.), pp. 125-142, 1996].

In general embodiments, lipolytic enzymes contemplated for use hydrolyze esters of glycerol based lipids (e.g., a triglyceride, a phospholipid). Glycerol is a naturally produced alcohol having a 3 carbon backbone with 3 alcohol moieties (positions 1, 2, and 3). One or more of these positions are often esterified with a fatty acid in many naturally produced or synthetic lipids. Common examples of triglycerides include a fat, which is solid at ambient conditions, or an oil, which is liquid at ambient conditions. As used herein, a “fatty acid” (“FA”) refers to saturated, monounsaturated or polyunsaturated aliphatic acids. They may be “short chain” (2-6 carbons), “medium chain” (8-14 carbons) or “long chain” (16 or more carbons, e.g., 40 carbons) aliphatic acids. Lipolytic enzymes hydrolyze esters at one or more of glycerol's alcohol positions (e.g., a 1, 3 lipase), though lipolytic enzymes often can hydrolyze non-glycerol esters of an alcohol other than glycerol. For example, naturally produced waxes are fatty acid esters of ethylene glycol, which has a 2 carbon backbone and 2 alcohol moieties, where one or both of the alcohol moieties are esterified with a fatty acid.

In other lipids, a fatty acid is esterified to an alcohol group of a non-glycerol or ethylene glycol molecule, such as sterol lipid (e.g., cholesterol), and an enzyme that catalyzes that linkage is considered herein (and in the art) to be a lipolytic enzyme (e.g., a sterol hydrolase). Conversely, in some cases, one or more positions of a glycerol, ethylene glycol or other alcohol comprise a fatty acid and other comprise an esters of a chemical structure other than fatty acids, and an enzyme that catalyzes hydrolysis or cleavage that non-FA linkage is considered herein (and in the art) a lipolytic enzyme (e.g., a phospholipase). For example, a phospholipid (“phosphoglyceride”) comprises a diglyceride with the 3 remaining position esterified to a phosphate group, which is esterified to a hydrophilic moiety such as a polyhydroxyl alcohol (e.g., glycerol, inositol) or amino alcohol (e.g., choline, serine, ethanolamine). Examples of phospholipids that lipolytic enzymes act on include phosphatidic acid (“PA”), phosphatidylcholine (“PC,” “lecithin”), phosphotidyl ethanolamine (“PE,” “cephalin”), phosphotidylglycerol (“PG”), phosphotidylinositol (“PI,” “monophosphoinositide”), phosphotidylserine (“PE,” “serine”), phosphotidylinositol 4,5-diphosphate (“PIP2,” “triphosphoinositide”), and diphosphotidylglycerol (“DPG,” “cardiolipin”). In some cases, a glycerol, ethylene glycol or other alcohol comprise a non-ester linkage to a fatty acid, and a lipolytic enzyme may act on that substrate to hydrolyze or cleave that linkage. For example, sphingomyelin comprises a glycerol having a fatty acid amide bond and 2 phosphate ester bonds, and a lipolytic enzyme may cleave the amide linkage.

Enzymes are identified and referred to by their primary catalytic function, but often catalyze other reactions, and multiple examples of such enzymes are referred to herein (e.g., a carboxylesterase/lipase). In some embodiments, one or more lipolytic enzymes in a surface treatment will possess the ability to cleave (e.g., hydrolyze) all positions of an alcohol for ease of removal of the products of the reaction. Mixture of lipolytic enzymes may be used to broaden the range of effective activity against various substrates or environmental conditions. In some embodiments, a multifunction enzyme may be used instead a plurality of enzymes to expand the range of different substrates that are hydrolyzed or degraded, though a plurality of single and/or multifunctional enzymes may be used as well.

Though lipolytic enzymes often produce a product that is more aqueous soluble or removable after a single chemical reaction, in some aspects, a series of enzyme reactions is needed to release a fatty acid or degrade a lipid, such as in the case of a combination of a sphingomyelin phosphodiesterase that produces a N-acylsphingosine from a sphingomyelin phospholipid, followed by a ceramidase hydrolyzing an amide bond in a N-acylsphingosine to produce a free fatty acid and a sphingosine.

Often a lipolytic enzyme will have preference for an isomer or enantiomer for a particular lipid (e.g., a triglyceride comprising one sequence of different fatty acids esters out of many that are possible), but in some embodiments one or more lipolytic enzymes in a coating or surface treatment will possess the ability to hydrolyze a plurality of lipid isomers or enantiomers for a broader range of substrates acted upon by a composition.

Petroleum hydrocarbons generally comprise a mixture of various alkanes, cycloalkanes, aromatic hydrocarbons, and polycyclic aromatic hydrocarbons. These lipids differ from the lipids catalyzed by alpha/beta hydrolases, in that they lack ester bonds, and lack chemical moieties such as an alcohol or acid. Some microorganisms are capable of digesting one or more of these types of lipids, generally by adding one or more oxygen moieties prior to integration of the lipid into cellular metabolic pathways. Often petroleum degradation occurs via a metabolic pathway comprising numerous enzymes and proteins, in some cases bound to cellular membranes. Such an enzyme or series of enzyme(s) and/or protein(s) that improves a petroleum hydrocarbon's aqueous solubility, absorption into a coating or film, or an effective combination thereof, is considered herein to be a lipolytic enzyme, and is known herein as a “petroleum lipolytic enzyme” to differentiate it from lipolytic enzymes that act on non-petroleum substrates described above.

Biomolecular compositions may be made from cells that produce such a petroleum lipolytic enzyme. A type of petroleum lipolytic enzyme is one that first adds, rather than modifies, an aqueous solubility enhancing moiety (e.g., an alcohol, an acid), as that initial modification in a degradation pathway is contemplated to be sufficient to improve the aqueous solubility and/or absorptive properties of the target petroleum lipid. As exemplified by the Pseudomonas putida alkane degradation pathway encoded by an alkBFGHIJKL operon, a petroleum alkane substrate undergoes catalysis by a plurality of enzymes (e.g., an alkane hydroxylase, a rubredoxins, an aldehyde dehydrogenase, an alcohol dehydrogenase, an acyl-CoA synthetase) and proteins (e.g., an outer membrane protein, a methyl-accepting transducer protein), that convert the alkane into an aldehyde and an acid with the participation of additional enzymes and proteins not encoded by the operon. A membrane bound monooxygenase, a rubredioxin, and a soluble rubredioxin add an alcohol moiety to the petroleum alkane by shunting electrons through a NADH compound to a hydroxylase. These initial enzymatic activities that result in improvement of aqueous solubility by addition of an alcohol may be used to select an enzyme. The alcohol is further catalyzed into an aldehyde, then an acid, before entering regular cellular metabolic pathways (e.g., energy production). Other pathways are thought to use a dioxygenase to initially produce an n-alkyl hydroperoxide that is converted into an aldehyde, using a flavin adenine dinucleotide, but not a NADPH or a rubredoxin (Van Hamme, J. D., 2003).

Another example of petroleum product degradation is a polycyclic aromatic hydrocarbon having oxygenated moieties added by the enzymes and proteins expressed from the nahAaAbAcAdBFCED operon for naphthalene degradation. These enzymes and proteins include: a reductase (nahAa), a ferredoxin (nahAb), an iron sulfur protein large subunit (nahAc), an iron sulfur protein small subunit (nahAd), a cis-naphthalene dihydrodiol dehydrogenase (nahB), a salicyaldehyde dehydrogenase (nahf), a 1,2-dihydroxynaphthalene oxygenase (nahC), a 2-hydroxybenzalpyruvate aldolase (nahE), a 2-hydroxychromene-2-carboxylate isomerase (nahD). The naphthalene dioxygenase is encoded by the nahAa to nahAd genes. Pseudomonas putida strains may also have the salicylate degradation pathway, which includes the following enzymes: salicylate hydroxylase (nahG), chloroplast-type ferredoxin (nahT), catechol oxygenase (nahH), 2-Hydroxymuconic semialdehyde dehydrogenase (nahI), 2-Hydroxymuconic semialdehyde dehydrogenase (nahN), 2-Oxo-4-pentenoate hydratase (nahL), 4-Hydroxy-2-oxovalerate aldolase (nahO), acetaldehyde dehydrogenase (nahM), 4-oxalocrotonate decarboxylase (nahK), and 2-hydroxymuconate tautomerase (nahJ). Both operons are regulated by salicylate induction of the nahR gene from another operon (Van Hamme, J. D., 2003).

As petroleum products are often mixtures of various linear and cyclical hydrocarbons, a plurality of petroleum lipolytic enzymes in a biomolecular composition (e.g., a plurality of cells that act one or more petroleum substrates, a plurality of semipurified or purified petroleum lipolytic enzymes, etc.) are contemplated to improve the solubility of many or all components of the petroleum product. In some embodiments, complete conversion of the petroleum product through all the steps of a petroleum degradation pathway is contemplated (e.g., via a cell-based composition comprising all the degradation pathway's enzymes).

In general embodiments, a lipolytic enzyme comprises a hydrolase. A hydrolase generally comprises an esterase, a ceramidase (EC 3.5.1.23), or a combination thereof. Examples of an esterase is those identified by enzyme commission number (EC 3.1): a carboxylic ester hydrolase, (EC 3.1.3), a phosphoric monoester hydrolase (EC 3.1.3), a phosphoric diester hydrolase (EC 3.1.4), or a combination thereof. As used herein, a “carboxylic ester hydrolase” catalyzes the hydrolytic cleavage of an ester to produce an alcohol and a carboxylic acid anion product. As used herein, a “phosphoric monoester hydrolase” catalyzes the hydrolytic cleavage of an O—P ester bond. As used herein, a “phosphoric diester hydrolase” catalyzes the hydrolytic cleavage of a phosphate group's phosphorus atom and two other moieties over two ester bonds. As used herein a “ceramidase” hydrolyzes the N-acyl bond of ceramide to release a fatty acid and sphingosine. Examples of a lipolytic esterase and a ceramidase include a carboxylesterase (EC 3.1.1.1), a lipase (EC 3.1.1.3), a lipoprotein lipase (EC 3.1.1.34), an acylglycerol lipase (EC 3.1.1.23), a hormone-sensitive lipase (EC 3.1.1.79), a phospholipase A₁ (EC 3.1.1.32), a phospholipase A₂ (EC 3.1.1.4), a phosphatidylinositol deacylase (EC 3.1.1.52), a phospholipase C (EC 3.1.4.3), a phospholipase D (EC 3.1.4.4), a phosphoinositide phospholipase C (EC 3.1.4.11), a phosphatidate phosphatase (EC 3.1.3.4), a lysophospholipase (EC 3.1.1.5), a sterol esterase (EC 3.1.1.13), a galactolipase (EC 3.1.1.26), a sphingomyelin phosphodiesterase (EC 3.1.4.12), a sphingomyelin phosphodiesterase D (EC 3.1.4.41), a ceramidase (EC 3.5.1.23), a wax-ester hydrolase (EC 3.1.1.50), a fatty-acyl-ethyl-ester synthase (EC 3.1.1.67), a retinyl-palmitate esterase (EC 3.1.1.21), a 11-cis-retinyl-palmitate hydrolase (EC 3.1.1.63), a all-trans-retinyl-palmitate hydrolase (EC 3.1.1.64), a cutinase (EC 3.1.1.74), an acyloxyacyl hydrolase (EC 3.1.1.77), a petroleum lipolytic enzyme, or a combination thereof.

a. Carboxylesterases

Carboxylesterase (EC 3.1.1.1) has been also referred to in that art as “carboxylic-ester hydrolase,” “ali-esterase,” “B-esterase,” “monobutyrase,” “cocaine esterase,” “procaine esterase,” “methylbutyrase,” “vitamin A esterase,” “butyryl esterase,” “carboxyesterase,” “carboxylate esterase,” “carboxylic esterase,” “methylbutyrate esterase,” “triacetin esterase,” “carboxyl ester hydrolase,” “butyrate esterase,” “methylbutyrase,” “α-carboxylesterase,” “propionyl esterase,” “nonspecific carboxylesterase,” “esterase D,” “esterase B,” “esterase A,” “serine esterase,” “carboxylic acid esterase,” and “cocaine esterase.” Carboxylesterase catalyzes the reaction: carboxylic ester+H₂O=an alcohol+a carboxylate. In many embodiments, the carboxylate comprises a fatty acid. In additional aspects, the FA comprises 10 or less carbons, to differentiate its preferred substrate and classification from a lipase, though a carboxylesterase (e.g., a microsome carboxylesterase) often will possess the catalytic activity of an arylesterase, a lysophospholipase, an acetylesterase, an acylglycerol lipase, an acylcarnitine hydrolase, a palmitoyl-CoA hydrolase, an amidase, an aryl-acylamidase, a vitamin A esterase, or combination thereof. Carboxylesterase producing cells and methods for isolating a carboxylesterase from cellular materials and biological sources have been described [see, for example, Augusteyn, R. C. et al., 1969; Horgan, D. J., et al., 1969; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for wild-type carboxylesterase and/or mutant/functional equivalent amino acid sequences for producing a carboxylesterase or a functional equivalent include Protein database bank entries: 1AUO, 1AUR, 1CI8, 1CI9, 1EVQ, 1JJI, 1K4Y, 1L7Q, 1L7R, 1MX1, 1MX5, 1MX9, 1QZ3, 1RID, 1TQH, 1U4N, 1YA4, 1YA8, 1YAH, 1YAJ, 2C7B, 2DQY, 2DQZ, 2DRO, 2FJO, 2H1I, 2H7C, 2HM7, 2HRQ, 2HRR, 2JEY, 2JEZ, 2JF0, 2O7R, 2O7V, 2OGS, 2OGT, and 2R11.

b. Lipases

Lipase (EC 3.1.1.3) has been also referred to in that art as “triacylglycerol acylhydrolase,” “triacylglycerol lipase,” “triglyceride lipase,” “tributyrase,” “butyrinase,” “glycerol ester hydrolase,” “tributyrinase,” “Tween hydrolase,” “steapsin,” “triacetinase,” “tributyrin esterase,” “Tweenase,” “amno N-AP,” “Takedo 1969-4-9,” “Meito MY 30,” “Tweenesterase,” “GA 56,” “capalase L,” “triglyceride hydrolase,” “triolein hydrolase,” “tween-hydrolyzing esterase,” “amano CE,” “cacordase,” “triglyceridase,” “triacylglycerol ester hydrolase,” “amano P.” “amano AP,” “PPL,” “glycerol-ester hydrolase,” “GEH,” “meito Sangyo OF lipase,” “hepatic lipase,” “lipazin,” “post-heparin plasma protamine-resistant lipase,” “salt-resistant post-heparin lipase,” “heparin releasable hepatic lipase,” “amano CES,” “amano B,” “tributyrase,” “triglyceride lipase,” “liver lipase,” and “hepatic monoacylglycerol acyltransferase.” Lipase catalyzes the reaction: triacylglycerol+H₂O=diacylglycerol+a carboxylate. In many embodiments, the carboxylate comprises a fatty acid. Lipase and/or co-lipase producing cells and methods for isolating a lipase and/or a co-lipase from cellular materials and biological sources have been described, [see, for example, Kom, E. D. and Quigley., 1957; Lynn, W. S, and Perryman, N. C., 1960; Tani, T. and Tominaga, Y. J., 1991; Sugihara, A. et al., 1992; in “Methods and Molecular Biology, Volume 109 Lipase and Phospholipase Protocols.” (Mark Doolittle and Karen Reue, Eds.), pp. 157-164, 1999; pancreatic lipase via recombinant expression in a baculoviral system in “Methods and Molecular Biology, Volume 109 Lipase and Phospholipase Protocols.” (Mark Doolittle and Karen Reue, Eds.), pp. 187-213, 1999; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974; “Lipases” (Borgstrom, B. and Brockman, H. L., Eds), p. 49-262, 307-328, 365-416, 1984; In “Lipases and Phospholipases in Drug Development from Biochemistry to Molecular Pharmacology.” (Müller, G. and Petry, S. Eds.) pp. 1-22, 2004], and may be used in conjunction with the disclosures herein.

A lipase can often catalyze the hydrolysis of short or medium chain FAs less than 12 carbons (“12C”), but has a preference or specificity for 12C or greater (e.g., long chain) FAs. In contrast, a lipolytic enzyme classified as a carboxylesterase prefers short or medium chain FAs, though some carboxylesterases can also hydrolyze esters of longer FAs. The chain length preference for lipase is generally applicable to the other preferred lipolytic FA esterases and ceramidase described herein, other than carboxylesterases unless otherwise noted.

Mammalian lipases are generally classified into four groups, gastric, hepatic, lingual, and pancreatic, and have homology to lipoprotein lipase. Pancreatic lipases are inactivated by bile salts, amphiphilic molecules found in animal intestines that are thought to bind lipids and confer a negative charge that inhibits a pancreatic lipase. Colipase is a protein that binds pancreatic lipase and reactivates it in the presence of bile salts [In “Engineering of/with Lipases” (F. Xavier Malcata., Ed.) p. 168, 1996]. In some embodiments, it is contemplated that a co-lipase will be combined with a pancreatic lipase in a composition to promote lipase (e.g., a pancreatic lipase) activity.

Structural information for wild-type lipase and/or mutant/functional equivalent amino acid sequences for producing a lipase or a functional equivalent include Protein database bank entries: 1AKN, 1BU8, 1CRL, 1CUA, 1CUB, 1CUC, 1CUD, 1CUE, 1CUF, 1CUG, 1CUH, 1CUI, 1CUJ, 1CUU, 1CUV, 1CUW, 1CUX, 1CUY, 1CUZ, 1CVL, 1DT3, 1DT5, 1DTE, 1DU4, 1EIN, 1ETH, 1EX9, 1F6W, 1FFA, 1FFB, 1FFC, 1FFD, 1FFE, 1GPL, 1GT6, 1GZ7, 1HLG, 1HPL, 1HQD, 1I6W, 1ISP, 1J13, 1JMY, 1K8Q, 1KUO, 1LBS, 1LBT, ILGY, 1LLF, 1LPA, 1LPB, 1LPM, 1LPN, 1LPO, 1LPP, 1LPS, 1N8S, 1OIL, 1QGE, 1R4Z, 1R50, 1RP1, 1T2N, 1T4M, 1TAH, 1TCA, 1TCB, 1TCC, 1TGL, 1THG, 1TIA, 1TIB, 1TIC, 1TRH, 1YS1, 1YS2, 2DSN, 2ES4, 2FX5, 2HIH, 2LIP, 2NW6, 2ORY, 2OXE, 2PPL, 2PVS, 2QUA, 2QUB, 2QXT, 2QXU, 2VEO, 2Z5G, 2Z8X, 2Z8Z, 3D2A, 3D2B, 3D2C, 3LIP, 3TGL, 4LIP, 4TGL, 5LIP, and 5TGL.

c. Lipoprotein Lipases

Lipoprotein lipase (EC 3.1.1.134) has been also referred to in that art as “triacylglycero-protein acylhydrolase,” “clearing factor lipase,” “diglyceride lipase,” “diacylglycerol lipase,” “postheparin esterase,” “diglyceride lipase,” “postheparin lipase,” “diacylglycerol hydrolase,” and “lipemia-clearing factor.” A lipoprotein lipase's biological function is to hydrolyze triglycerides found in animal lipoproteins. Lipoprotein lipase catalyzes the reaction: triacylglycerol+H₂O=diacylglycerol+a carboxylate. This enzyme also acts on diacylglycerol to produce a monoacylglycerol. Apolipoprotein activates lipoprotein lipase [“Lipases” (Borgstrom, B. and Brockman, H. L., Eds), p. 228-230, 1984]. In some embodiments, a protein such as apolipoprotein may be combined with a lipoprotein lipase. Lipoprotein lipase producing cells and methods for isolating a lipoprotein lipase from cellular materials and biological sources have been described, [see, for example, Egelrud, T. and Olivecrona, T., 1973; Greten, H. et al., 1970; in “Methods and Molecular Biology, Volume 109 Lipase and Phospholipase Protocols.” (Mark Doolittle and Karen Reue, Eds.), pp. 133-143, 1999; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974; “Lipases” (Borgstrom, B. and Brockman, H. L., Eds), p. 263-306, 1984], and may be used in conjunction with the disclosures herein.

d. Acylglycerol Lipases

Acylglycerol lipase (EC 3.1.1.23) has been also referred to in that art as “glycerol-ester acylhydrolase,” “monoacylglycerol lipase,” “monoacylglycerolipase,” “monoglyceride lipase,” “monoglyceride hydrolase,” “fatty acyl monoester lipase,” “monoacylglycerol hydrolase,” “monoglyceridyllipase,” and “monoglyceridase.” Acylglycerol lipase catalyzes a glycerol monoester's hydrolysis, particularly a FA ester's hydrolysis. Acylglycerol lipase producing cells and methods for isolating an acylglycerol lipase from cellular materials and biological sources have been described, [see, for example, Mentlein, R. et al., 1980; Pope, J. L. et al., 1966; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

e. Hormone-Sensitive Lipases

Hormone-sensitive lipase (EC 3.1.1.79) has been also referred to in that art as “diacylglycerol acylhydrolase” and “HSL.” Hormone-sensitive lipase catalyzes the reactions, in order of catalytic preference: diacylglycerol+H₂O=monoacylglycerol+a carboxylate; triacylglycerol+H₂O=diacylglycerol+a carboxylate; and monoacylglycerol+H₂O=glycerol+a carboxylate. A hormone-sensitive lipase generally is also active against steroid fatty acid esters and retinyl esters, and/or has a preference for 1- or 3-ester bond of acylglycerol substrates. Hormone-sensitive lipase producing cells and methods for isolating a hormone-sensitive lipase from cellular materials and biological sources have been described, [see, for example, Tsujita, T. et al., 1989; Fredrikson, G., et al., 1981; via recombinant expression in a baculoviral system in “Methods and Molecular Biology, Volume 109 Lipase and Phospholipase Protocols.” (Mark Doolittle and Karen Reue, Eds.), pp. 165-175, 1999; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

f. Phospholipases A

Phospholipase A₁ (EC 3.1.1.32) has been also referred to in that art as “phosphatidylcholine 1-acylhydrolase.” Phospholipase A₁ catalyzes the reaction: phosphatidylcholine+H2O=2-acylglycerophosphocholine+a carboxylate. Phospholipases A₁ substrate specificity generally is broader than phospholipase A₂, and typically needs Ca²⁺ for improved activity. Phospholipase A₁ producing cells and methods for isolating a phospholipase A₁ from cellular materials and biological sources have been described [see, for example, Gatt, S., 1968; van den Bosch, H., et al., 1974; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for wild-type phospholipase A₁ and/or mutant/functional equivalent amino acid sequences for producing a phospholipase A₁ or a functional equivalent include Protein database bank entries: 1FW2, 1FW3, 1ILD, 1ILZ, 1IM0, 1QD5, and 1QD6.

g. Phospholipases A

Phospholipase A₂ (EC 3.1.1.4) has been also referred to in that art as “phosphatidylcholine 2-acylhydrolase,” “lecithinase A,” “phosphatidase,” “phosphatidolipase,” ad “phospholipase A.” Phospholipase A₂ catalyzes the reaction: phosphatidylcholine+H₂O=1-acylglycerophosphocholine+a carboxylate. Phospholipases A₂ also catalyzes reactions on phosphatidylethanolamine, choline plasmalogen and phosphatides, and acts on the 2-position ester bonds. Ca²⁺ is generally needed for improved enzyme function. Phospholipase A₂ producing cells and methods for isolating a phospholipase A₂ from cellular materials and biological sources have been described, [see, for example, Saito, K. and Hanahan, D. J., 1962; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for wild-type phospholipase A₂ and/or mutant/functional equivalent amino acid sequences for producing a phospholipase A₂ or a functional equivalent include Protein database bank entries: 1A2A, 1A3D, 1A3F, 1AE7, 1AOK, 1AYP, 1B4W, 1BBC, 1BCI, 1BJJ 1BK9, 1BP2, 1BPQ, 1BUN, 1BVM, 1C1J, 1C74, 1CEH, 1CJY, 1CL51CLP, 1DB4, 1DB5, 1DCY, 1DPY, 1FAZ, 1FDK, 1FE5, 1FX9, 1FXF 1G0Z, 1G2X, 1G41, 1 GH4, 1GMZ, IGOD, 1GP7, 1HN4, 1IJL, 1IRB 1IT4, 1IT5, 1J1A, 1J1A, 1JLT, 1JQ8, 1JQ9, 1KP4, 1KPM, 1KQU 1KVO, 1KVW, 1KVX, 1KVY, 1L8S, 1LE6, 1LE7, 1LN8, 1LWB, 1M8R 1M8S, 1M8T, 1MF4, 1MG6, 1 MH2, 1 MH7, 1 MH8, 1MKS, 1MKT, 1MKU 1MKV, 1N28, 1N29, 1O2E, 1O3W, 1OQS, 1OWS, 1OXL, 1OXR, 1OYF 1OZ6, 1OZY, 1P2P, 1P7O, 1PA0, 1PC9, 1PIR, 1PIS, 1P08, 1POA 1POB, 1POC, 1POD, 1POE, 1PP2, 1PPA, 1PSH, 1PSJ, 1PWO, 1Q6V 1Q7A, 1QLL, 1RGB, 1RLW, 1S6B, 1S8G, 1S8H, 1S81, 1SFV, 1SFW 1SKG, 1SQZ, 1SV3, 1SV9, 1SXK, 1SZ8, 1T37, 1TC8, 1TD7, 1TDV 1TG1, 1TG4, 1TGM, 1TH6, 1TJ9, 1TJK, 1TJQ, 1TK4, 1TP2, 1U4J1U73, 1UNE, IVAP, 1VIP, 1VKQ, IVL9, 1XXS, 1XXW, 1Y38, 1Y4L 1Y60, 1Y6P, 1Y75, 1YXH, 1YXL, 1Z76, 1ZL7, 1ZLB, 1ZM6, 1ZR81ZWP, 1ZYX, 2ARM, 2AZY, 2AZZ, 2B00, 2B01, 2B03, 2B04, 2B17 2B96, 2BAX, 2BCH, 2BD1, 2BPP, 2DO2, 2DPZ, 2DV8, 2FNX, 2G58 2GNS, 2H₄C, 210U, 2NOT, 2O1N, 2OL1, 2OQD, 2OSH, 2OSN, 2OTF 2OTH, 2OUB, 2OYF, 2PB8, 2PHI, 2PMJ, 2PVT, 2PWS, 2PYC, 2Q1P 2QHD, 2QHE, 2QHW, 2QOG, 2QU9, 2QUE, 2QVD, 2RD4, 2ZBH, 3BJW 3BP2, 3CBI, 3P2P, 4BP2, 4P2P, and 5P2P.

h. Phosphatidylinositol Deacylases

Phosphatidylinositol deacylase (EC 3.1.1.152) has been also referred to in that art as “1-phosphatidyl-D-myo-inositol 2-acylhydrolase,” “phosphatidylinositol phospholipase A₂,” and “phospholipase A2.” Phosphatidylinositol deacylase catalyzes the reaction: 1-phosphatidyl-D-myo-inositol+H₂O=1-acylglycerophosphoinositol+a carboxylate. Phosphatidylinositol deacylase producing cells and methods for isolating a phosphatidylinositol deacylase from cellular materials and biological sources have been described, [see, for example, Gray, N. C. C. and Strickland, K. P., 1982; Gray, N. C. C. and Strickland, K. P., 1982; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

i. Phospholipases C

Phospholipase C (EC 3.1.4.3) has been also referred to in that art as “phosphatidylcholine cholinephosphohydrolase,” “lipophosphodiesterase I,” “lecithinase C,” “Clostridium welchii α-toxin,” “Clostridium oedematiens β- and γ-toxins,” “lipophosphodiesterase C,” “phosphatidase C,” “heat-labile hemolysin,” and “α-toxin.” Phospholipase C catalyzes the reaction: phosphatidylcholine+H₂O=1,2-diacylglycerol+choline phosphate. A bacterial phospholipase C may have activity against sphingomyelin and phosphatidylinositol. Phospholipase C producing cells and methods for isolating a phospholipase C from cellular materials and biological sources have been described [see, for example, Sheiknejad, R. G. and Srivastava, P. N., 1986; Takahashi, T., et al., 1974; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for wild-type phospholipase C and/or mutant/functional equivalent amino acid sequences for producing a phospholipase C or a functional equivalent include Protein database bank entries: 1AH7, 1CA1, 1GYG, 1IHJ, 1OLP, 1P5X, 1P6D, 1P6E, 1 QM6, 1 QMD, 2FFZ, 2FGN, and 2HUC.

j. Phospholipases D

Phospholipase D (EC 3.1.4.4) has been also referred to in that art as “phosphatidylcholine phosphatidohydrolase,” “lipophosphodiesterase II,” “lecithinase D,” and “choline phosphatase.” Phospholipase D catalyzes the reaction: phosphatidylcholine+H₂O=choline+a phosphatidate. A phospholipase D may have activity against other phosphatidyl esters. Phospholipase D producing cells and methods for isolating a phospholipase D from cellular materials and biological sources have been described, [see, for example, Astrachan, L. 1973; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for wild-type phospholipase D and/or mutant/functional equivalent amino acid sequences for producing a phospholipase D or a functional equivalent include Protein database bank entries: 1F0I, 1V0R, 1V0S, 1V0T, 1V0U, 1V0V, 1V0W, 1V0Y, 2ZE4, and 2ZE9.

k. Phosphoinositide Phospholipase C

Phosphoinositide phospholipase C (EC 3.1.4.11) has been also referred to in that art as “1-phosphatidyl-1D-myo-inositol-4,5-bisphosphate inositoltrisphosphohydrolase,” “triphosphoinositide phosphodiesterase,” “phosphoinositidase C,” “1-phosphatidylinositol-4,5-bisphosphate phosphodiesterase,” “monophosphatidylinositol phosphodiesterase,” “phosphatidylinositol phospholipase C,” “PI-PLC,” and “1-phosphatidyl-D-myo-inositol-4,5-bisphosphate inositoltrisphosphohydrolase.” Phosphoinositide phospholipase C catalyzes the reaction: 1-phosphatidyl-1D-myo-inositol 4,5-bisphosphate+H₂O=1D-myo-inositol 1,4,5-trisphosphate+diacylglycerol. A phosphoinositide phospholipase C may have activity against other phosphatidyl esters. Phosphoinositide phospholipase C producing cells and methods for isolating a phosphoinositide phospholipase C from cellular materials and biological sources have been described, [see, for example, Downes, C. P. and Michell, R. H.1981; Rhee, S. G. and Bae, Y. S. 1997; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for wild-type phosphoinositide phospholipase C and/or mutant/functional equivalent amino acid sequences for producing a phosphoinositide phospholipase C or a functional equivalent include Protein database bank entries: 1DJG, 1DJH, 1DJI, 1DJW, 1DJX, 1DJY, 1DJZ, 1HSQ, 1JAD, 1MAI, 1QAS, 1QAT, 1Y0M, 1YWO, 1YWP, 2C5L, 2EOB, 2FCI, 2FJL, 2FJU, 2HSP, 2ISD, 2K2J, 2PLD, 2PLE, and 2ZKM.

l. Phosphatidate Phosphatases

Phosphatidate phosphatase (EC 3.1.3.4) has been also referred to in that art as “3-sn-phosphatidate phosphohydrolase,” “phosphatic acid phosphatase,” “acid phosphatidyl phosphatase,” and “phosphatic acid phosphohydrolase.” A phosphatidate phosphatase catalyzes the reaction: 3-sn-phosphatidate+H₂O=a 1,2-diacyl-sn-glycerol+phosphate. A phosphatidate phosphatase may have activity against other phosphatidyl esters. Phosphatidate phosphatase producing cells and methods for isolating a phosphatidate phosphatase from cellular materials and biological sources have been described, [see, for example, Smith, S. W., et al., 1957; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

m. Lysophospholipases

Lysophospholipase (EC 3.1.1.5) has been also referred to in that art as “2-lysophosphatidylcholine acylhydrolase,” “lecithinase B.” “lysolecithinase,” “phospholipase B,” “lysophosphatidase,” “lecitholipase,” “phosphatidase B.” “lysophosphatidylcholine hydrolase,” “lysophospholipase A1,” “lysophopholipase L2,” “lysophospholipaseDtransacylase,” “neuropathy target esterase,” “NTE,” “NTE-LysoPLA,” and “NTE-lysophospholipase.” A lysophospholipase catalyzes the reaction: 2-lysophosphatidylcholine+H₂O=glycerophosphocholine+a carboxylate. Lysophospholipase producing cells and methods for isolating a lysophospholipase from cellular materials and biological sources have been described, [see, for example, van den Bosch, H., et al., 1981; van den Bosch, H., et al., 1973; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein. Structural information for wild-type lysophospholipase and/or mutant/functional equivalent amino acid sequences for producing a lysophospholipase or a functional equivalent include Protein database bank entries: 1G86, 1HDK, 1TVN, 1J00, 1JRL, 1LCL, 1QKQ, 1U8U, 1V2G, 2G07, 2G08, 2G09, and 2GOA.

n. Sterol Esterases

Sterol esterase (EC 3.1.1.13) has been also referred to in that art as “lysosomal acid lipase,” “sterol esterase,” “cholesterol esterase,” “cholesteryl ester synthase,” “triterpenol esterase,” “cholesteryl esterase,” “cholesteryl ester hydrolase,” “sterol ester hydrolase,” “cholesterol ester hydrolase,” “cholesterase,” and “acylcholesterol lipase.” A sterol esterase catalyzes the reaction: steryl ester+H₂O=a sterol+a fatty acid. A sterol esterase is often active against triglycerides as well. Cholesterol is generally the substrate used to characterize a sterol esterase, though the enzyme also hydrolyzes lipid vitamin esters (e.g., Vitamin E acetate, Vitamin E palmate, Vitamin D₃ acetate). Bile salts often activate the enzyme. Sterol esterase producing cells and methods for isolating a sterol esterase from cellular materials and biological sources have been described [see, for example, Okawa, Y. and Yamaguchi, T., 1977; via recombinant expression in a baculoviral system in “Methods and Molecular Biology, Volume 109 Lipase and Phospholipase Protocols.” (Mark Doolittle and Karen Reue, Eds.), pp. 177-186, 203-213, 1999; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974; “Lipases” (Borgstrom, B. and Brockman, H. L., Eds), p. 329-364, 1984.], and may be used in conjunction with the disclosures herein. Structural information for wild-type sterol esterase and/or mutant/functional equivalent amino acid sequences for producing a sterol esterase or a functional equivalent include Protein database bank entries: 1AQL, and 2BCE.

o. Galactolipases

Galactolipase (EC 3.1.1.26) has been also referred to in that art as “1,2-diacyl-3-β-D-galactosyl-sn-glycerol acylhydrolase,” “galactolipid lipase,” “polygalactolipase,” and “galactolipid acylhydrolase.” A galactolipase catalyzes the reaction: 1,2-diacyl-3-β-D-galactosyl-sn-glycerol+2H₂O=3-β-D-galactosyl-sn-glycerol+2 carboxylates. A galactolipase also may have activity against phospholipids. The substrate for galactolipase is the galactolipids abundantly found in plant cells, and organisms that digest plant material (e.g., animals) also produce this enzyme. Galactolipase producing cells and methods for isolating a galactolipase from cellular materials and biological sources have been descibed, [see, for example, Helmsing, 1969; Hirayama, O., et al., 1975 In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

p. Sphingomyelin Phosphodiesterases

Sphingomyelin phosphodiesterase (EC 3.1.4.12) has been also referred to in that art as “sphingomyelinase,” “neutral sphingomyelinase,” “sphingomyelin cholinephosphohydrolase,” and “sphingomyelin N-acylsphingoosine-hydrolase.” A sphingomyelin phosphodiesterase catalyzes the reaction: sphingomyelin+H₂O=N-acylsphingosine+choline phosphate. A sphingomyelin phosphodiesterase also may have activity against phospholipids. Sphingomyelin phosphodiesterase producing cells and methods for isolating a sphingomyelin phosphodiesterase from cellular materials and biological sources have been described, [see, for example, Chatterjee, S, and Ghosh, N. 1989; Kanfer, J. N., et al., 1966; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

q. Sphingomyelin Phosphodiesterases D

Sphingomyelin phosphodiesterase D (EC 3.1.4.41) has been also referred to in that art as “sphingomyelin ceramide-phosphohydrolase” and “sphingomyelinase D.” A sphingomyelin phosphodiesterase D catalyzes the reaction: sphingomyelin+H₂O=ceramide phosphate+choline. A sphingomyelin phosphodiesterase D also may catalyze the reaction: hydrolyses 2-lysophosphatidylcholine to choline and 2-lysophosphatidate. Sphingomyelin phosphodiesterase D producing cells and methods for isolating a sphingomyelin phosphodiesterase D from cellular materials and biological sources have been described, [see, for example, Soucek, A. et al., 1971; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

r. Ceramidases

Ceramidase (EC 3.5.1.23) has been also referred to in that art as “N-acylsphingosine amidohydrolase,” “acylsphingosine deacylase,” and “glycosphingolipid ceramide deacylase sphingomyelin.” A ceramidase catalyzes the reaction: N-acylsphingosine+H₂O=a carboxylate+sphingosine. Ceramidase producing cells and methods for isolating a ceramidase from cellular materials and biological sources have been described [see, for example, E. and Gatt, S., 1969; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

s. Wax-Ester Hydrolases

Wax-ester hydrolase (EC 3.1.1.50) has been also referred to in that art as “wax-ester acylhydrolase,” and “jojoba wax esterase,” and “WEH.” A wax-ester hydrolase catalyzes the reaction: wax ester+H₂O=a long-chain alcohol+a long-chain carboxylate. A wax-ester hydrolase may also hydrolyze a long-chain acylglycerol. Wax-ester hydrolase producing cells and methods for isolating a wax-ester hydrolase from cellular materials and biological sources have been described, [see, for example, Huang, A. H. C. et al., 1978; Moreau, R. A. and Huang, A. H. C., 1981; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

t. Fatty-Acyl-Ethyl-Ester Synthases

Fatty-acyl-ethyl-ester synthase (EC 3.1.1.67) has been also referred to in that art as “long-chain-fatty-acyl-ethyl-ester acylhydrolase,” and “FAEES.” A fatty-acyl-ethyl-ester synthase catalyzes the reaction: long-chain-fatty-acyl ethyl ester+H₂O=a long-chain-fatty acid+ethanol. Fatty-acyl-ethyl-ester synthase producing cells and methods for isolating a fatty-acyl-ethyl-ester synthase from cellular materials and biological sources have been described [see, for example, Mogelson, S, and Lange, L. G. 1984; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

u. Retinyl-Palmitate Esterases

Retinyl-palmitate esterase (EC 3.1.1.21) has been also referred to in that art as “retinyl-palmitate palmitohydrolase,” “retinyl palmitate hydrolase,” “retinyl palmitate hydrolyase,” and “retinyl ester hydrolase.” A retinyl-palmitate esterase catalyzes the reaction: retinyl palmitate+H₂O=retinol+palmitate. A retinyl-palmitate esterase may also hydrolyze a long-chain acylglycerol. Retinyl-palmitate esterase producing cells and methods for isolating a retinyl-palmitate esterase from cellular materials and biological sources have been described, [see, for example, T. et al., 2005; Gao, J. and Simon, 2005; Brockerhoff, Hans and Jensen, Robert G. “Lipolytic Enzymes,” 1974], and may be used in conjunction with the disclosures herein.

v. 11-cis-Retinyl-Palmitate Hydrolases

11-cis-retinyl-palmitate hydrolase (EC 3.1.1.63) has been also referred to in that art as “11-cis-retinyl-palmitate acylhydrolase,” “11-cis-retinol palmitate esterase,” and “RPH.” An 11-cis-retinyl-palmitate hydrolase catalyzes the reaction: 11-cis-retinyl palmitate+H₂O=11-cis-retinol+palmitate. 11-cis-retinyl-palmitate hydrolase producing cells and methods for isolating a 11-cis-retinyl-palmitate hydrolase from cellular materials and biological sources have been described, [see, for example, Blaner, W. S., et al., 1987; Blaner, W. S., et al., 1984; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

w. All-Trans-Retinyl-Palmitate Hydrolases

All-trans-retinyl-palmitate hydrolase (EC 3.1.1.64) has been also referred to in that art as “all-trans-retinyl-palmitate acylhydrolase.” All-trans-retinyl-palmitate hydrolase catalyzes the reaction: all-trans-retinyl palmitate+H₂O=all-trans-retinol+palmitate. Detergent generally promotes this enzyme's activity. All-trans-retinyl-palmitate hydrolase producing cells and methods for isolating a All-trans-retinyl-palmitate hydrolase from cellular materials and biological sources have been described, [see, for example, Blaner, W. S., Das, et al., 1987; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

x. Cutinases

Cutinase (EC 3.1.1.74) has been also referred to in that art as “cutin hydrolase.” A cutinase catalyzes the reaction: cutin+H₂O=cutin monomers. A cutinase also has lipase and/or carboxylesterase activity noted for not needing interfacial activation. Cutinase isolation producing cells and methods for isolating a cutinase from cellular materials and biological sources have been described, [see, for example, Garcia-Lepe, R., et al., 1997; Purdy, R. E. and Kolattukudy, P. E., 1975; Sebastian, J., and Kolattukudy, P. E., 1988; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974; “Lipases” (Borgstrom, B. and Brockman, H. L., Eds), p. 471-504, 1984], and may be used in conjunction with the disclosures herein.

y. Acyloxyacyl Hydrolases

An acyloxyacyl hydrolase (EC 3.1.1.77) catalyzes the reaction: 3-(acyloxy)acyl group of bacterial toxin=3-hydroxyacyl group of bacterial toxin+a fatty acid. An acyloxyacyl hydrolase generally prefers Salmonella typhimurium and related organisms lipopolysaccharides as substrates. However, an acyloxyacyl hydrolase may also possess phospholipase, acyltransferase, phospholipase A₂, lysophospholipase, phospholipase A₁, phosphatidylinositol deacylase, diacylglycerol lipase, and/or phosphatidyl lipase activity. An acyloxyacyl hydrolase generally prefers saturated C₁₂-C₁₆ FA esters. Acyloxyacyl hydrolase producing cells and methods for isolating an acyloxyacyl hydrolase from cellular materials and biological sources have been descibed, [see, for example, Hagen, F. S., et al., 1991; Munford, R. S, and Hunter, J. P., 1992; In “Lipases their Structure, Biochemistry and Application” (Paul Woolley and Steffen B. Peterson, Eds.), pp. 243-270, 1994; Brockerhoff, Hans and Jensen, Robert G. “Lipolytic Enzymes,” 1974], and may be used in conjunction with the disclosures herein.

3. Additional Enzymes: Phosphoric Triester Hydrolases

In some embodiments, the additional enzyme comprises a hydrolase. An additional hydrolase may comprise an esterase. A type of additional esterase comprises an esterase that catalyzes the hydrolysis of an organophosphorus compound. Examples of such additional esterases are those identified by enzyme commission number EC 3.1.8, the phosphoric triester hydrolases. As used herein, a phosphoric triester hydrolase catalyzes the hydrolytic cleavage of an ester from a phosphorus moiety. Examples of a phosphoric triester hydrolase include an aryldialkylphosphatase, a diisopropyl-fluorophosphatase, or a combination thereof. It is contemplated that a coating or surface treatment with dual functions, ease of lipid and organophosphorus compound removal/detoxification, will be of benefit depending upon the type of compounds that contact such a composition.

In some embodiments, an enzyme possesses both a lipolytic and an organophosphorus compound binding or organophosphorus compound hydrolytic activities, and such a multifunctional enzyme is contemplated for use. For example, some carboxylesterases (e.g., Rattus norvegicus ES4, ES10; enzyme isolates from Myzus persicae; and Homo sapiens liver cells) have demonstrated this binding and/or catalytic property against soman or malathion. Often the organophosphorus compound acts as an inhibitor of the carboxylesterase, though hydrolysis occurs in some instances [In “Esterases, Lipases, and Phospholipases from Structure to Clinical Significance.” (Mackness, M. I. and Clerc, M., Eds.), pp. 91-98, 1994]. As some carboxylesterases are contemplated as being more suitable for lipolytic activity, and others for organophosphorus compound binding or hydrolytic activities, they are differentiated herein by the use of “carboxylesterase” when referring to an enzyme as a lipolytic enzyme, and a “carboxylase” when referring to an enzyme as an organophosphorus compound degrading enzyme, particularly in the claims.

An aryldialkylphosphatase (EC 3.1.8.1) is also known by its systemic name “aryltriphosphate dialkylphosphohydrolase,” and various enzymes in this category have been known in the art by names such as “organophosphate hydrolase”; “paraoxonase”; “A-esterase”; “aryltriphosphatase”; “organophosphate esterase”; “esterase B1”; “esterase E4”; “paraoxon esterase”; “pirimiphos-methyloxon esterase”; “OPA anhydrase”; “organophosphorus hydrolase”; “phosphotriesterase”; “PTE”; “paraoxon hydrolase”; “OPH”; and “organophosphorus acid anhydrase.” An aryldialkylphosphatase catalyzes the following reaction: aryl dialkyl phosphate+H₂O=an aryl alcohol+dialkyl phosphate. Examples of an aryl dialkyl phosphate include an organophosphorus compound comprising a phosphonic acid ester, a phosphinic acid ester, or a combination thereof.

A diisopropyl-fluorophosphatase (EC 3.1.8.2) is also known by its systemic name “diisopropyl-fluorophosphate fluorohydrolase,” and various enzymes in this category have been known in the art by names such as “DFPase”; “tabunase”; “somanase”; “organophosphorus acid anhydrolase”; “organophosphate acid anhydrase”; “OPA anhydrase”; “diisopropylphosphofluoridase”; “dialkylfluorophosphatase”; “diisopropyl phosphorofluoridate hydrolase”; “isopropylphosphorofluoridase”; and “diisopropylfluorophosphonate dehalogenase.” A diisopropyl-fluorophosphatase catalyzes the following reaction: diisopropyl fluorophosphate+H₂O=fluoride+diisopropyl phosphate. Examples of a diisopropyl fluorophosphates include an organophosphorus compound comprising a phosphorus-halide, a phosphorus-cyanide, or a combination thereof.

Examples of phosphoric triester hydrolases and cleaved OP compounds and bond types are shown at Table 1.

TABLE 1
Phosphoric Triester Hydrolases

| | OP Compound Phosphoryl Bond-Type and | | | | |
|---|---|---|---|---|---|
| | Phosphoryl Bond Types Cleaved by Enzyme | | | | |
| | | Various OP | Sarin, | VX, | |
| | | Pesticides | Soman | R-VX | Tabun |
| Enzyme | P—C | P—O | P—F | P—S | P—CN |
| OPH^{a,b,c,d,e,f,g} | − | + | + | + | + |
| Human | + | + | + | − | + |
| Paraoxonase^{h,i,j} | | | | | |
| OPAA-2^{k,l} | − | + | + | − | + |
| Squid DFPase^{m} | − | − | + | − | − |

^{a}Dumas D. P. et al., 1989a;
^{b}Dumas, D. P. et al., 1989b;
^{c}Dumas D. P. et al., 1990;
^{d}Dave, K. I. et al., 1993;
^{e}Chae, M. Y. et al., 1994;
^{f}Lai, K. et al., 1995;
^{g}Kolakowski, J. E. et al., 1997;
^{h}Hassett, C. et al., 1991;
ⁱJosse, D. et al., 2001;
^{j}Josse, D. et al., 1999;
^{k}DeFrank, J. J. et al. 1993;
^{l}Cheng, T.-C. et al., 1996;
^{m}Hoskin, F. C. G. and Roush, A. H., 1982.

An additional substrate for a composition comprises an organophosphorus compound. As used herein, an “organophosphorus compound” is a compound comprising a phosphoryl center, and further comprises two or three ester linkages. In some aspects, the type of phosphoester bond and/or additional covalent bond at the phosphoryl center classifies an organophosphorus compound. In embodiments wherein the phosphorus is linked to an oxygen by a double bond (P═O), the OP compound is known as an “oxon OP compound” or “oxon organophosphorus compound.” In embodiments wherein the phosphorus is linked to a sulfur by a double bond (P═S), the OP compound is known as a “thion OP compound” or “thion organophosphorus compound.” Additional examples of bond-type classified OP compounds include a phosphonocyanate, which comprises a P—CN bond; a phosphoroamidate, which comprises a P—N bond; a phosphotriester, which comprises a P—O bond; a phosphodiester, which comprises a P—O bond; a phosphonofluoridate, which comprises a P—F bond; and a phosphonothiolate, which comprises a P—S bond. A “dimethyl OP compound” comprises two methyl moieties covalently bonded to the phosphorus atom, such as, for example, malathion. A “diethyl OP compound” comprises two ethoxy moieties covalently bonded to the phosphorus atom, such as, for example, diazinon.

In general embodiments, an OP compound comprises an organophosphorus nerve agent or an organophosphorus pesticide. As used herein, a “nerve agent” is an inhibitor of a cholinesterase, including but not limited to, an acetyl cholinesterase, a butyl cholinesterase, or a combination thereof. The toxicity of an OP compound depends on the rate of release of its phosphoryl center (e.g., P—C, P—O, P—F, P—S, P—CN) from the target enzyme (Millard, C. B. et al., 1999). In specific embodiments, a nerve agent is an inhibitor of a cholinesterase (e.g., acetyl cholinesterase) whose catalytic activity is often needed for health and survival in animals, including humans.

Certain OP compounds are so toxic to humans that they have been adapted for use as chemical warfare agents, such as tabun, soman, sarin, cyclosarin, VX, and R-VX. A CWA may be in airborne form and such a formulation is known herein as an “OP-nerve gas.” Examples of airborne forms include a gas, a vapor, an aerosol, a dust, or a combination thereof. Examples of an OP compounds that may be formulated as an OP nerve gas include tabun, sarin, soman, VX, GX, or a combination thereof.

In addition to the initial inhalation route of exposure common to such agents, CWAs, especially persistent agents such as VX and thickened soman, pose threats through dermal absorption [In “Chemical Warfare Agents: Toxicity at Low Levels,” (Satu M. Somani and James A. Romano, Jr., Eds.) p. 414, 2001]. As used herein, a “persistent agent” is a CWA formulated to be non-volatile and thus remain as a solid or liquid while exposed to the open air for more than three hours. Often after release, a persistent agent may convert from an airborne dispersal form to a solid or liquid residue on a surface, thus providing the opportunity to contact the skin of a human. The toxicities for common OP chemical warfare agents after contact with skin are shown at Table 2.

TABLE 2
LD₅₀ Values* of Common Organophosphorus
Chemical Warfare Agents

| | Estimated human LD₅₀ - percutaneous (skin) |
|---|---|
| Common OP CWA | administration |
| Tabun | 1000 milligrams (“mg”) |
| Sarin | 1700 mg |
| Soman | 100 mg |
| VX | 10 mg |

*LD₅₀ - the dose need to kill 50% of individuals in a population after administration, wherein the individuals weigh approximately 70 kg.

In some embodiments, an OP compound may be a particularly poisonous organophosphorus nerve agent. As used herein, a “particularly poisonous” agent is a composition with a LD₅₀ of 35 mg/kg or less for an organism after percutaneous (“skin”) administration of the agent. Examples of a particularly poisonous OP nerve agent include tabun, sarin, cyclosarin, soman, VX, R-VX, or a combination thereof.

As used herein, “detoxification,”. “detoxify,” “detoxified,” “degradation,” “degrade,” and “degraded” refers to a chemical reaction of a compound that produces a chemical byproduct that is less harmful to the health or survival of a target organism contacted with the chemical product relative to contact with the parent compound. OP compounds may be detoxified using chemical hydrolysis or through enzymatic hydrolysis (Yang, Y.-C. et al., 1992; Yang, Y.-C. et al., 1996; Yang, Y.-C. et al., 1990; LeJeune, K. E. et al., 1998a). In general embodiments, the enzymatic hydrolysis is a specifically targeted reaction wherein the OP compound is cleaved at the phosphoryl center's chemical bond resulting in predictable byproducts that are acidic in nature but benign from a neurotoxicity perspective (Kolakowski, J. E. et al., 1997; Rastogi, V. K. et al., 1997; Dumas, D. P. et al., 1990; Raveh, L. et al., 1992). By comparison, chemical hydrolysis can be much less specific, and in the case of VX may produce some quantity of byproducts that approach the toxicity of the intact agent (Yang, Y.-C. et al., 1996; Yang, Y.-C. et al., 1990). In facets, an enzyme composition degrades a CWA, a particularly poisonous organophosphorus nerve agent, or a combination thereof into byproduct that is not particularly poisonous.

Many OP compounds are pesticides that are not particularly poisonous to humans, though they do possess varying degrees of toxicity to humans and other animals. Examples of an OP pesticide include bromophos-ethyl, chlorpyrifos, chlorfenvinphos, chlorothiophos, chlorpyrifos-methyl, coumaphos, crotoxyphos, crufomate, cyanophos, diazinon, dichlofenthion, dichlorvos, dursban, EPN, ethoprop, ethyl-parathion, etrimifos, famphur, fensulfothion, fenthion, fenthrothion, isofenphos, jodfenphos, leptophos-oxon, malathion, methyl-parathion, mevinphos, paraoxon, parathion, parathion-methyl, pirimiphos-ethyl, pirimiphos-methyl, pyrazophos, quinalphos, ronnel, sulfopros, sulfotepp, trichloronate, or a combination thereof. In some embodiments, a composition degrades a pesticide into a byproduct that is less toxic to an organism. In specific aspects, the organism is an animal, such as a human.

a. OPH

Organophosphorus hydrolase (E.C.3.1.8.1) has been also referred to in that art as “organophosphate-hydrolyzing enzyme,” “phosphotriesterase,” “PTE,” “organophosphate-degrading enzyme,” “OP anhydrolase,” “OP hydrolase,” “OP thiolesterase,” “organophosphorus triesterase,” “parathion hydrolase,” “paraoxonase,” “DFPase,” “somanase,” “VXase,” and “sarinase.” As used herein, this type of enzyme will be referred to herein as “organophosphorus hydrolase” or “OPH.”

The initial discovery of OPH was from two bacterial strains from the closely related genera: Pseudomonas diminuta and Flavobacterium spp. (McDaniel, S. et al., 1988; Harper, L. et al., 1988), which encoded identical organophosphorus degrading opd genes on large plasmids (Genbank accession no. M20392 and Genbank accession no. M22863) (copending U.S. patent application Ser. No. 07/898,973, incorporated herein in its entirety by reference). It is likely that Pseudomonas diminuta was derived from the Flavobacterium spp. Subsequently, other such OPH encoding genes have been discovered. The use of any opd gene or their gene product in the described compositions and methods is contemplated. Examples of opd genes and gene products that may be used include the Agrobacterium radiobacter P230 organophosphate hydrolase gene, opdA (Genbank accession no. AY043245; Entrez databank no. AAK85308); the Flavobacterium balustinum opd gene for parathion hydrolase (Genbank accession no. AJ426431; Entrez databank no. CAD19996); the Pseudomonas diminuta phosphodiesterase opd gene (Genbank accession no. M20392; Entrez databank no. AAA98299; Protein Data Bank entries 1JGM, 1DPM, 1EYW, 1EZ2, 1HZY, 1IOB, 1IOD, 1PSC and 1PTA); the Flavobacterium sp opd gene (Genbank accession no. M22863; Entrez databank no. AAA24931; ATCC 27551); the Flavobacterium sp. parathion hydrolase opd gene (Genbank accession no. M29593; Entrez databank no. AAA24930; ATCC 27551); or a combination thereof (Home, I. et al., 2002; Somara, S. et al., 2002; McDaniel, C. S. et al., 1988a; Harper, L. L. et al., 1988; Mulbry, W. W. and Karns, J. S., 1989).

Because OPH possesses the property of cleaving a broad range of OP compounds (Table 1), it is the OP detoxifying enzyme that has been often studied and characterized, with the enzyme obtained from Pseudomonas being the target of focus for many studies. This OPH was initially purified following expression from a recombinant baculoviral vector in insect tissue culture of the Fall Armyworm, Spodoptera frugiperda (Dumas, D. P. et al., 1989b). Purified enzyme preparations have been shown to be able to detoxify via hydrolysis a wide spectrum of structurally related insect and mammalian neurotoxins that function as acetylcholinesterase inhibitors. Of great interest, this detoxification ability included a number of organophosphorofluoridate nerve agents such as sarin and soman. This was the first recombinant DNA construction encoding an enzyme capable of degrading these potent nerve gases. This enzyme was capable of degrading the common organophosphorus insecticide analog (paraoxon) at rates exceeding 2×10⁷ M⁻¹ (mole enzyme)⁻¹, which is equivalent to the catalytically efficient enzymes observed in nature. The purified enzyme preparations are capable of detoxifying sarin and the less toxic model mammalian neurotoxin O,O-diisopropyl phosphorofluoridate (“DFP”) at the equivalent rates of 50-60 molecules per molecule of enzyme-dimer per second. In addition, the enzyme can hydrolyze soman and VX at approximately 10% and 1% of the rate of sarin, respectively. The breadth of substrate utility (e.g., V agents, sarin, soman, tabun, cyclosarin, OP pesticides) and the efficiency for the hydrolysis exceeds the known abilities of other prokaryotic and eukaryotic organophosphorus acid anydrases, and it is clear that this detoxification is due to a single enzyme rather than a family of related, substrate-limited proteins.

The X-ray crystal structure of Pseudomonas OPH has been determined (Benning, M. M. et al., 1994; Benning, M. M. et al., 1995; Vanhooke, J. L. et al., 1996). Each OPH monomer's active site binds two atoms of Zn²⁺; however, OPH is usually prepared wherein Co²⁺ replaces Zn²⁺, which enhances catalytic rates. Examples of the catalytic rates (k_{cat}) and specificities (k_{cat}/Kₘ) for Co²⁺ substituted OPH against various OP compounds are shown at Table 3 below.

TABLE 3
Catalytic Activity of Wild-Type OPH binding Co²⁺

| | k_{cat} (s⁻¹) | k_{cat}/Kₘ (M⁻¹ s⁻¹) |
|---|---|---|
| OP Pesticide Substrate | | |
| Paraoxon | 15000^{a} | 1.3 × 10⁸ |
| OP CWA Substrates | | |
| Sarin | 56^{b} | 8 × 10⁴ |
| Soman | 5^{b} | 1 × 10⁴ |
| VX | 0.3^{b} | 7.5 × 10² |
| R-VX | 0.5^{c} | 105 |
| Tabun* | 77^{d} | 7.6 × 10⁵ |

*Wild-type Zn²⁺ OPH was used in obtaining these kinetic parameters;
^{a}diSioudi, B. et al., 1999a;
^{b}Kolakoski, J. E. et al., 1997;
^{c}Rastogi, V. K. et al., 1997;
^{d}Raveh, L. et al., 1992.

The phosphoryl center of OP compounds is chiral, and Pseudomonas OPH preferentially binds and/or cleaves Sₚ enantiomers over Rₚ enantiomers of the chiral phosphorus in various substrates by a ratio of about 10:1 to about 90:1 (Chen-Goodspeed, M. et al., 2001a; Hong, S.-B. and Raushel, F. M., 1999a; Hong, S.-B. and Raushel, F. M., 1999b). CWAs such as VX, sarin, and soman are usually prepared and used as a mixture of sterioisomers of varying toxicity, with VX and sarin having two enantomers each, with the chiral center around the phosphorus of the cleavable bond. Soman possesses four enantomers, with one chiral center based on the phosphorus and an additional chiral center based on a pinacolyl moeity [In “Chemical Warfare Agents: Toxicity at Low Levels” (Satu M. Somani and James A. Romano, Jr., Eds.) pp 26-29, 2001; Li, W.-S. et al., 2001; Yang, Y.-C. et al., 1992; Benshop, H. P. et al., 1988]. The S_{P} enantiomer of sarin is about 10⁴ times faster in inactivating acetylcholinesterase than the R_{P} enantiomer (Benschop, H. P. and De Jong, L. P. A. 1988), while the two Sₚ enantiomers of soman is about 10⁵ times faster in inactivating acetylcholinesterase than the R_{P} enantiomers (Li, W.-S. et al., 2001; Benschop, H. P. et al., 1984). Wild-type organophosphorus hydrolase seems to have greater specificity for the less toxic enantiomers of sarin and soman. OPH is about 9-fold faster cleaving an analog of the R_{P} enantiomer of sarin relative to an analog of the Sₚ enantiomer, and about 10-fold faster in cleaving analogs of the R_{c} enantiomers of soman relative to analogs of the S_{c} enantiomers (Li, W.-S. et al., 2001).

b. Paraoxonase

Human paraoxonase (EC 3.1.8.1), is a calcium dependent protein, and is also known as an “arylesterase” or aryl-ester hydrolase” (Josse, D. et al., 1999; Vitarius, J. A. and Sultanos, L. G., 1995). Examples of the human paraoxonase (“HPON1”) gene and gene products can be accessed at (Genbank accession no. M63012; Entrez databank no. AAB59538) (Hassett, C. et al., 1991).

c. Carboxylases

It is contemplated that a carboxylase gene isolated from an animal may be used as an organophosphate hydrolase. As used herein, a “carboxylase” or “ali-esterase” (EC 3.1.1.1) is an enzyme that hydrolytically cleaves carboxylic esters (e.g., C—O bonds). Many genes in eukaryatic organisms have multiple alleles which comprise varient nucleotide and/or expressed protein sequences for a particular gene. Certain insect species have been identified with reduced carboxylase activity and enhanced resistance to OP compounds such as malathion or diazinon. Examples of insect species include Plodia interpunctella, Chrysomya putoria, Lucilia cuprina, and Musca domestica. In particular, an allele of a carboxylase gene possessing organophosphate hydrolase (EC 3.1.8.1) activity is thought to be responsible for OP compound resistance. Examples of such carboxylase genes include alleles isolated from Lucilia cuprina (Genbank accession no. U56636; Entrez databank no. AAB67728), Musca domestica (Genbank accession no. AF133341; Entrez databank no. AAD29685), or a combination thereof (Claudianos, C. et al., 1999; Campbell, P. M. et al., 1998; Newcomb, R. D. et al., 1997). Additionally, carboxylases or carbamoyl lyases are useful against the carbamate nerve agents, and are specifically contemplated for use in biomolecular composition for use against such agents.

d. OPAAs, Prolidases, Aminopeptideases and PepQ

Organophosphorus acid anhydrolases (E.C.3.1.8.2), known as “OPAAs,” have been isolated from microorganisms and identified as enzymes that detoxify OP compounds (Serdar, C. M. and Gibson, D. T., 1985; Mulbry, W. W. et al., 1986; DeFrank, J. J. and Cheng, T.-C., 1991). The better-characterized OPAAs have been isolated from Altermonas species, such as Alteromonas sp JD6.5, Alteromonas haloplanktis and Altermonas undina (ATCC 29660) (Cheng, T.-C. et al., 1996; Cheng, T.-C. et al., 1997; Cheng, T. C. et al., 1999; Cheng, T.-C. et al., 1993). Examples of OPAA genes and gene products that may be used include the Alteromonas sp JD6.5 opaA gene, (GeneBank accession no. U29240; Entrez databank no. AAB05590); the Alteromonas haloplanktis prolidase gene (GeneBank accession no. U56398; Entrez databank AAA99824; ATCC 23821); or a combination thereof (Cheng, T. C. et al., 1996; Cheng, T.-C. et al., 1997). The wild-type encoded OPAA from Alteromonas sp JD6.5 is 517 amino acids, while the wild-type encoded OPAA from Alteromonas haloplanktis is 440 amino acids (Cheng, T. C. et al., 1996; Cheng, T.-C. et al., 1997). The Alteromonas OPAAs accelerates the hydrolysis of phosphotriesters and phosphofluoridates, including cyclosarin, sarin and soman (Table 4).

TABLE 4
Catalytic Activity of Wild-Type OPAAS

| | k_{cat} (s⁻¹) per species | | |
|---|---|---|---|
| | OPAA per OP Substrate | | |
| | A. sp JD6.5 | A. haloplanktis | A. undina |
| OP Compound Substrate | | | |
| DFP | 1650^{a} | 575^{a} | 1239^{a} |
| OP CWA Substrates | | | |
| Sarin | 611^{a} | 257^{a} | 376^{a} |
| Cyclosarin | 1650^{a} | 269^{a} | 1586^{a} |
| Soman | 3145^{a} | 1389^{a} | 2496^{a} |
| Tabun | 85^{a} | 113^{a} | 292^{a} |

^{a}Cheng, T. C. et al.,

Similar to OPH, OPAA from Alteromonas sp JD6.5 (“OPAA-2”) has a general binding and cleavage preference up to 112:1 for the Sp enantiomers of various p-nitrophenyl phosphotriesters (Hill, C. M. et al., 2000). Additionally, OPAA from Alteromonas sp JD6.5 is over 2 fold faster at cleaving an Sₚ enantiomer of a sarin analog, and over 15-fold faster in cleaving analogs of the R_{c} enantiomers of soman relative to analogs of the S_{c} enantiomers (Hill, C. M. et al., 2001).

Additionally, a prolidase (“imidodipeptidase,” “proline dipeptidase,” “peptidase D,” “g-peptidase”), PepQ and/or aminopeptidase P gene or gene product with OPAA activity, or a functional equivalent thereof may be used. OPAAs possess sequence and structural similarity to human prolidase, Escherichia coli aminopeptidase P and Escherichia coli PepQ (Cheng, T.-C. et al., 1997; Cheng, T.-C. et al., 1996). A prolidase or a PepQ protein (E.C. 3.4.13.9) hydrolyzes a C—N bond of a dipeptide with a prolyl residue at the carboxyl-terminus, and OPAAs are also classified as prolidases. An aminopeptidase P (EC 3.4.11.9) hydrolyzes the C—N amino bond of a proline at the penultimate position from the amino terminus of an amino acid sequence. Partly purified human and porcine prolidase demonstrated the ability to cleave DFP and G-type nerve agents (Cheng, T.-C. et. al., 1997). Examples of prolidase genes and gene products include the Mus musculus prolidase gene (GeneBank accession no. D82983; Entrez databank no. BAB11685); the Homo sapien prolidase gene (GeneBank accession no. J04605; Entrez databank AAA60064); the Lactobacillus helveticus prolidase (“PepQ”) gene (GeneBank accession no. AF012084; Entrez databank AAC24966); the Escherichia coli prolidase (“pepQ”) gene (GeneBank accession no. X54687; Entrez databank CAA38501); the Escherichia coli aminopeptidase P (“pepP”) gene (GeneBank accession no. D00398; Entrez databank BAA00299; Protein Data Bank entries 1A16, 1AZ9, 1JAW and 1M35); or a combination thereof (Ishii, T. et al., 1996; Endo, F. et al., 1989; Nakahigashi, K. and Inokuchi, H., 1990; Yoshimoto, T. et al., 1989).

e. Squid-Type DFPases

As used herein, a “squid-type DFPase” (EC 3.1.8.2) refers to an enzyme that catalyzes the cleavage of both DFP and soman, and is isolated from organisms of the Loligo genus. Generally, a squid-type DFPase cleaves DFP at a faster rate than soman. Squid-type DFPases include, for example, a DFPase from Loligo vulgaris, Loligo pealei, Loligo opalescens, or a combination thereof (Hoskin, F. C. G. et al., 1984; Hoskin, F. C. G. et al., 1993; Garden, J. M. et al., 1975).

A well-characterized example of a squid-type DFPase includes the DFPase that has been isolated from the optical ganglion of Loligo vulgaris (Hoskin, F. C. G. et al., 1984). This squid-type DFPase cleaves a variety of OP compounds, including DFP, sarin, cyclosarin, soman, and tabun (Hartleib, J. and Ruterjans, H., 2001a). The gene encoding this squid-type DFP has been isolated, and can be accessed at GeneBank accession no. AX018860 (International patent publication: WO 9943791-A). Further, this enzyme's X-ray crystal structure has been determined (Protein Data Bank entry 1E1A) (Koepke, J. et al., 2002; Scharff, E. I. et al., 2001). This squid-type DFPase binds two Ca²⁺ ions, which function in catalytic activity and enzyme stability (Hartleib, J. et al., 2001). Both the DFPase from Loligo vulgaris and Loligo pealei are susceptible to proteolytic cleavage into a 26-kDa and 16 kDa fragments, and the fragments from Loligo vulgaris are capable of forming active enzyme when associated together (Hartleib, J. and Ruterjans, H., 2001a).

f. Mazur-Type DFPases

As used herein, a “Mazur-type DFPase” (EC 3.1.8.2) refers to an enzyme that catalyzes the cleavage of both DFP and soman. Generally, Mazur-type DFPases cleaves soman at a faster rate than DFP. Examples of a Mazur-type DFPases include the DFPase isolated from mouse liver (Billecke, S. S. et al., 1999), which may be the same as the DFPase known as SMP-30 (Fujita, T. et al., 1996; Billecke, S. S. et al., 1999; Genebank accession no. U28937; Entrez databank AAC52721); a DFPase isolated from rat liver (Little, J. S. et al., 1989); a DFPase isolated from hog kidney; a DFPase isolated from Bacillus stearothermophilus strain OT, a DFPase isolated from Escherichia coli (ATCC25922) (Hoskin, F. C. G. et al., 1993; Hoskin, F. C. G, 1985); or a combination thereof.

g. Other Phosphoric Triester Hydrolases

It is contemplated that any phosphoric triester hydrolase that is known in the art may be used in additional embodiments. An example of an additional phosphoric triester hydrolase includes the product of the gene, mpd, (GenBank accession number AF338729; Entrez databank AAK14390) isolated from Plesiomonas sp. strain M6 (Zhongli, C. et al., 2001). Other examples include the phosphoric triester hydrolase identified in a Xanthomonas sp. (Tchelet, R. et al., 1993); Tetrahymena (Landis, W. G. et al., 1987); certain plants such as Myriophyllum aquaticum, Spirodela origorrhiza L, Elodea Canadensis and Zea mays (Gao, J. et al., 2000; Edwards, R. and Owen, W. J., 1988); and in hen liver and brain (Diaz-Alejo, N. et al., 1998). Additional, cholinesterases (e.g., an acetyl cholinesterase) with OP degrading activity have been identified in insects resistant OP pesticides (see, for example, Baxter, G. D. et al., 1998; Baxter, G. D. et al., 2002; Rodrigo, L., et al., 1997, Vontas, J. G., et al., 2002; Walsh, S. B., et al., 2001; Zhu, K. Y., et al., 1995), and are contemplate for use a bimolecular composition.

4. Additional Enzymes: Sulfuric Ester Hydrolases

A sulfuric ester hydrolase (EC 3.1.6) catalyzes the hydrolysis of a sulfuric ester bond. Examples of a sulfuric ester hydrolase include an arylsulfatase (EC 3.1.6.1), a steryl-sulfatase (EC 3.1.6.2), a glycosulfatase (EC 3.1.6.3), aN-acetylgalactosamine-6-sulfatase (EC 3.1.6.4), a choline-sulfatase (EC 3.1.6.6), a cellulose-polysulfatase (EC 3.1.6.7), a cerebroside-sulfatase (EC 3.1.6.8), a chondro-4-sulfatase (EC 3.1.6.9), a chondro-6-sulfatase (EC 3.1.6.10), a disulfoglucosamine-6-sulfatase (EC 3.1.6.11), a N-acetylgalactosamine-4-sulfatase (EC 3.1.6.12), an iduronate-2-sulfatase (EC 3.1.6.13), a N-acetylglucosamine-6-sulfatase (EC 3.1.6.14), a N-sulfoglucosamine-3-sulfatase (EC 3.1.6.15), a monomethyl-sulfatase (EC 3.1.6.16), a D-lactate-2-sulfatase (EC 3.1.6.17), a glucuronate-2-sulfatase (EC 3.1.6.18), or a combination thereof. An example of a sulfuric ester hydrolase is a an arylsulfatase (EC 3.1.6.1), which has been also referred to as “sulfatase,” “nitrocatechol sulfatase,” “phenolsulfatase,” phenylsulfatase,” “p-nitrophenyl sulfatase,” “arylsulfohydrolase,” “4-methylumbelliferyl sulfatase,” “estrogen sulfatase,” “arylsulfatase C,” “arylsulfatase B,” “arylsulfatase A,” and aryl-sulfate sulfohydrolase.” Arylsulfatase catalyzes the reaction: phenol sulfate+H₂O=a phenol+sulfate. As with other sulfuric ester hydrolases, arylsulfatase producing cells and methods for isolating an arylsulfatase from cellular materials and biological sources have been descibed, [see, for example, Dodgson, K. S. et al., 1956; Roy, A. B. 1960; Roy, A. B., 1976; Webb, E. C. and Morrow, P. F. W., 1959), and may be used in conjunction with the disclosures herein.

5. Additional Enzymes: Peptidases

A peptidase catalyzes a reaction on a peptide bond, though other reactions (e.g., esterase activity) may also be catalyzed in some cases. A peptidase generally may be categorized as either an exopeptidase (EC 3.4.11-19) or an endopeptidase (EC 3.4.21-24 and EC 3.4.99). Examples of a peptidase include an alpha-amino-acyl-peptide hydrolase (EC 3.4.11), a peptidyl-amino-acid hydrolase (EC 3.4.17), a dipeptide hydrolase (EC 3.4.13), a peptidyl peptide hydrolase (EC 3.4), a peptidylamino-acid hydrolase (EC 3.4), an acylamino-acid hydrolase (EC 3.4), an aminopeptidase (EC 3.4.11), a dipeptidase (EC 3.4.13), a dipeptidyl-peptidase (EC 3.4.14), a tripeptidyl-peptidase (EC 3.4.14), a peptidyl-dipeptidase (EC 3.4.15), a serine-type carboxypeptidase (EC 3.4.16), a metallocarboxypeptidase (EC 3.4.17), a cysteine-type carboxypeptidase (EC 3.4.18), an omega peptidase (EC 3.4.19), a serine endopeptidase (EC 3.4.21), a cysteine endopeptidase (EC 3.4.22), an aspartic endopeptidase (EC 3.4.23), a metalloendopeptidase (EC 3.4.24), a threonine endopeptidase (EC 3.4.25), an endopeptidases of unknown catalytic mechanism (EC 3.4.99), or a combination thereof. An example of a peptidase is chymotrypsin (EC 3.4.21.1) which has been also referred to as “chymotrypsins A and B,” “α-chymar ophth,” “avazyme,” “chymar,” “chymotest,” “enzeon,” “quimar,” “quimotrase,” “α-chymar,” “α-chymotrypsin A,” “α-chymotrypsin.” Chymotrypsin generally cleaves peptide bonds at the carboxyl side of amino acids, with a preference for Tyr, Trp, Phe, or Leu comprising substrates. As with other peptidases, chymotrypsin producing cells and methods for isolating an acyloxyacyl hydrolase from cellular materials and biological sources have been descibed, [see, for example, Dodgson, K. S. et al., 1956; Roy, A. B. 1960; Roy, A. B., 1976; Webb, E. C. and Morrow, P. F. W., 1959), and may be used in conjunction with the disclosures herein.

6. Additional Enzymes: Peroxidases

Peroxidase (EC 1.11.1.7; CAS registry number: 9003-99-0) has been also referred to as “myeloperoxidase,” “lactoperoxidase,” “verdoperoxidase,” “guaiacol peroxidase,” “thiocyanate peroxidase,” “eosinophil peroxidase,” “Japanese radish peroxidase,” “horseradish peroxidase (HRP),” “extensin peroxidase,” “heme peroxidase,” “MPO,” “oxyperoxidase,” “protoheme peroxidase,” “pyrocatechol peroxidase,” “scopoletin peroxidase,” and “donor:hydrogen-peroxide oxidoreductase.” Peroxidase catalyzes a reaction of hydrogen peroxide on a substrate (“donor”) to add an oxygen moiety via the reaction: donor+H2O2=oxidized donor+2H2O. Peroxidase is generally a hemoprotein. Peroxidase isolation producing cells and methods for isolating a peroxidase from cellular materials and biological sources have been described, [see, for example, Kenten, R. H. and Mann, P. J. G. Biochem. J. 57:347-348, 1954; Morrison, M. et al., J. Biol. Chem. 228:767-776, 1957; Paul, K. G. Peroxidases. In: Boyer, P. D., Lardy, H. and Myrbäck, K. (Eds.), The Enzymes, 2nd ed., vol. 8, Academic Press, New York, p. 227-274, 1963; Tagawa, K. et al., Nature (Lond.) 183:111, 1959; Theorell, H. Ark. Kemi Mineral. Geol. 16A No. 2. 11pp, 1943.], and may be used in conjunction with the disclosures herein.

7. Additional Enzymes: Trypsin

Trypsin (EC 3.4.21.4; CAS registry number: 9002-07-7) has been also referred to in that art as “α-trypsin;” “β-trypsin;” “cocoonase;” “parenzyme;” “parenzymol;” “tryptar;” “trypure;” “pseudotrypsin;” “tryptase;” “tripcellim;” “sperm receptor hydrolase.” Trypsin catalyzes the reaction: a preferential cleavage at Arg or Lys residues. Trypsin producing cells and methods for isolating a trypsin from cellular materials and biological sources have been described [see, for example, Huber, R. and Bode, W., 1978; Walsh, K. A., 1970; Read, R. J. et al., 1984; Fiedler, F. 1987; Fletcher, T. S. et al., 1987; Polgár, L. Structure and function of serine proteases. In New Comprehensive Biochemistry Vol. 16, Hydrolytic Enzymes (Neuberger, A. and Brocklehurst, K. eds), pp. 159-200, 1987; Tani, T., et al. 1990), and may be used in conjunction with the disclosures herein.

7. Functional Equivalents of Wild-Type Enzymes

It is possible to improve a proteinaceous molecule with a defined amino acid sequence and/or length for one or more properties. An alteration in a property is possible because such molecules can be manipulated, for example, by chemical modification, including but not limted to modifications described herein. As used herein “alter” or “alteration” may result in an increase or a decrease in the measured value for a particular property. As used herein a “property,” in the context of an proteinaceous molecule, includes, but is not limited to, a ligand binding property, a catalytic property, a stability property, a property related to environmental safety, or a combination thereof. Examples of a catalytic property that may be altered include a kinetic parameter, such as Kₘ, a catalytic rate (k_{cat}) for a substrate, an enzyme's specificity for a substrate (k_{cat}/Kₘ), or a combination thereof. Examples of a stability property that may be altered include thermal stability, half-life of activity, stability after exposure to a weathering condition, or a combination thereof. Examples of a property related to environmental safety include an alteration in toxicity, antigenicity, biodegradability, or a combination thereof. However, an alteration to increase an enzyme's catalytic rate for a substrate, an enzyme's specificity for a substrate, a proteinaceous molecule's thermal stability, a proteinaceous molecule's half-life of activity, or a proteinaceous molecule's stability after exposure to a weathering condition may be selected for some applications, while a decrease in toxicity and/or antigenicity for a proteinaceous molecule may be selected in additional applications. An enzyme comprising a chemical modification that functions as an enzyme is a “functional equivalent” to, and “in accordance” with, an unmodified enzyme.

It is also understood by those of skill in the art that there is a limit to the number of chemical modifications that can be made to an enzyme before a property is undesirably altered. However, in light of the disclosures herein of assays for determining whether a composition possesses one or more properties, including, for example, a enzymatic activity, a stability property, etc., using, but not limited to the assays described herein, to determine whether a given chemical modification to an enzyme produces a molecule that still possesses a suitable set of properties for use in a particular application. In certain aspects, a functional equivalent enzyme comprising a plurality of different chemical modifications can be produced.

It is particularly contemplated that a functional equivalent enzyme comprising a structural analog and/or sequence analog may possess an enhanced property and/or a reduced undesirable property, in comparison to the enzyme upon which it is based. As used herein, a “structural analog” refers to one or more chemical modifications to the peptide backbone or non-side chain chemical moieties of a proteinaceous molecule. In certain aspects, a subcomponent of an enzyme such as an apo-enzyme, a prosthetic group, a co-factor, or a combination thereof, may be modified to produce a functional equivalent structural analog. In particular facets, such an enzyme sub-component that does not comprise a proteinaceous molecule may be altered to produce a functional equivalent structural analog of an enzyme when combined with the other sub-components. As used herein, a “sequence analog” refers to one or more chemical modifications to the side chain chemical moieties, also known herein as a “residue” of one or more amino acids that define a proteinaceous molecule's sequence. Often such a “sequence analog” comprises an amino acid substitution, which is generally produced by recombinant expression of a nucleic acid comprising a genetic mutation to produce a mutation in the expressed amino acid sequence.

As used herein, an “amino acid’ may be a common or uncommon amino acid. The common amino acids include: alanine (Ala, A); arginine (Arg, R); aspartic acid (a.k.a. aspartate; Asp, D); asparagine (Asn, N); cysteine (Cys, C); glutamic acid (a.k.a. glutamate; Glu, E); glutamine (Gln, Q); glycine (Gly, G); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V). Common amino acids are often biologically produced in the biological synthesis of a peptide or a polypeptide. An uncommon amino acid refers to an analog of a common amino acid, as well as a synthetic amino acid whose side chain is chemically unrelated to the side chains of the common amino acids. Various uncommon amino acids may be used, though it is contemplated that in general embodiments, an enzyme will be biologically produced, and thus lack or possess relatively few uncommon amino acids prior to any subsequent non-mutation based chemical modifications.

The side chains of amino acids comprise moieties with specific chemical and physical properties. Certain side chains contribute to a ligand binding property, a catalytic property, a stability property, a property related to environmental safety, or a combination thereof. For example, cysteines can form covalent bonds between different parts of a contiguous amino acid sequence, or between non-contiguous amino acid sequences to confer enhanced stability to a secondary, tertiary or quaternary structure. In an additional example, the presence of hydrophobic or hydrophilic side chains exposed to the outer environment can alter the hydrophobicity or hydrophilicity of part of a proteinaceous sequence such as in the case of a transmembrane domain that is embedded in a lipid layer of a membrane. In another example, hydrophilic side chains may be exposed to the environment surrounding a proteinaceous molecule, which can enhance the overall solubility of a proteinaceous molecule in a polar liquid, such as water or a liquid component of a coating. In a further example, various acidic, basic, hydrophobic, hydrophilic, and/or aromatic side chains present at or near a binding site of a proteinaceous structure can affect the affinity for a proteinaceous sequence for binding a ligand and/or a substrate, based on the covalent, ionic, Van der Waal forces, hydrogen bond, hydrophilic, hydrophobic, and/or aromatic interactions at a binding site. Such interactions by residues at or near an active site also contribute to a chemical reaction that occurs at the active site of an enzyme to produce enzymatic activity upon a substrate. As used herein, a residue is “at or near” another residue or group of residues when it is within 15 Å, 14 Å, 13 Å, 12 Å, 11 Å, 10 Å, 9 Å, 8 Å, 7 Å, 6 Å, 5 Å, 4 Å, 3 Å, 2 Å, or 1 Å the residue or group of residues such as residues identified as contributing to the active site and/or binding site.

Identification of an amino acid whose chemical modification would likely change a property of a proteinaceous molecule can be accomplished using such methods as a chemical reaction, mutation, X-ray crystallography, nuclear magnetic resonance (“NMR”), computer based modeling or a combination thereof. Selection of an amino acid on the basis of such information can then be used in the rational design of a mutant proteinaceous sequence that would possess an altered property. Alterations include those that alter enzymatic activity to produce a functional equivalent of an enzyme.

For example, many residues of a proteinaceous molecule that contribute to the properties of a proteinaceous molecule comprise chemically reactive moieties. These residues are often susceptible to chemical reactions that can inhibit their ability to contribute to a property of the proteinaceous molecule. Thus, a chemical reaction can be used to identify one or more amino acids comprised within the proteinaceous molecule that may contribute to a property. The identified amino acids then can be subject to modifications such as amino acid substitutions to produce a functional equivalent. Examples of amino acids that can be so chemically reacted include Arg, which can be reacted with butanedione; Arg and/or Lys, which can be reacted with phenylglyoxal; Asp and/or Glu, which can be reacted with carbodiimide and HCl; Asp and/or Glu, which can be reacted with N-ethyl-5-phenylisoxazolium-3′-sulfonate (“Woodward's reagent K”); Asp and/or Glu, which can be reacted with 1,3-dicyclohexyl carbodiimide; Asp and/or Glu, which can be reacted with 1-ethyl-3-(3-dimethylaminopropyl)carbodiimide (“EDC”); Cys, which can be reacted with p-hydroxy mercuribenzoate; Cys, which can be reacted with dithiobisnitrobenzoate (“DTNB”); Cys, which can be reacted with iodoacetamide; His, which can be reacted with diethylpyrocarbonate (“DEPC”); His, which can be reacted with diazobenzenesulfonic acid (“DBS”); His, which can be reacted with 3,7-bis(dimethylamino)phenothiazin-5-ium chloride (“methylene blue”); Lys, which can be reacted with dimethylsuberimidate; Lys and/or Arg, which can be reacted with 2,4-dinitrofluorobenzene; Lys and/or Arg, which can be reacted with trinitrobenzene sulfonic acid (“TNBS”); Trp, which can be reacted with 2-hydroxy-5-nitrobenzyl bromide 1-ethyl-3 (3-dimethylaminopropyl); Trp, which can be reacted with 2-acetoxy-5-nitrobenzyl chloride; Trp, which can be reacted with N-bromosucinimide; Tyr, which can be reacted with N-acetyl]midazole (“NAI”); or a combination thereof (Hartleib, J. and Ruterjans, H., 2001b; Josse, D. et al., 1999; Josse, D. et al., 2001).

In an additional example, the secondary, tertiary and/or quaternary structure of a proteinaceous molecule may be modeled using techniques known in the art, including X-ray crystallography, nuclear magnetic resonance, computer based modeling, or a combination thereof to aid in the identification of active-site, binding site, and other residues for the design and production of a mutant form of an enzyme (Bugg, C. E. et al., 1993; Cohen, A. A. and Shatzmiller, S. E., 1993; Hruby, V. J., 1993; Moore, G. J., 1994; Dean, P. M., 1994; Wiley, R. A. and Rich, D. H., 1993). The secondary, tertiary and/or quaternary structures of a proteinaceous molecule may be directly determined by techniques such as X-ray crystallography and/or nuclear magnetic resonance to identify amino acids likely to effect one or more properties. Additionally, many primary, secondary, tertiary, and/or quaternary structures of proteinaceous molecules can be obtained using a public computerized database. An example of such a databank that may be used for this purpose is the Protein Data Bank (PDB), which is an international repository of the 3-dimensional structures of many biological macromolecules.

Computer modeling can be used to identify amino acids likely to affect one or more properties. Often, a structurally related proteinaceous molecule comprises primary, secondary, tertiary and/or quaternary structures that are evolutionarily conserved in the wild-type protein sequences of various organisms. The secondary, tertiary and/or quaternary structure of a proteinaceous molecule can be modeled using a computer to overlay the proteinaceous molecule's amino acid sequence, which is also known as the “primary structure,” onto the computer model of a described primary, secondary, tertiary, and/or quaternary structure of another, structurally related proteinaceous molecule. Often the amino acids that may participate in an active site, a binding site, a transmembrane domain, the general hydrophobicity and/or hydrophilicity of a proteinaceous molecule, the general positive and/or negative charge of a proteinaceous molecule, etc, may be identified by such comparative computer modeling.

In embodiments wherein an amino acid of particular interest has been identified using such techniques, functional equivalents may be created using mutations that substitute a different amino acid for the identified amino acid of interest. Examples of substitutions of an amino acid side chain to produce a “functional equivalent” proteinaceous molecule are also known in the art, and may involve a conservative side chain substitution a non-conservative side chain substitution, or a combination thereof, to rationally alter a property of a proteinaceous molecule. Examples of conservative side chain substitutions include, when applicable, replacing an amino acid side chain with one similar in charge (e.g., an arginine, a histidine, a lysine); similar in hydropathic index; similar in hydrophilicity; similar in hydrophobicity; similar in shape (e.g., a phenylalanine, a tryptophan, a tyrosine); similar in size (e.g., an alanine, a glycine, a serine); similar in chemical type (e.g., acidic side chains, aromatic side chains, basic side chains); or a combination thereof. Conversely, when a change to produce a non-conservative substitution is contemplated to alter a property of proteinaceous molecule, and still produce a “functional equivalent” proteinaceous molecule, these guidelines can be used to select an amino acid whose side-chains relatively non-similar in charge, hydropathic index, hydrophilicity, hydrophobicity, shape, size, chemical type, or a combination thereof. Various amino acids have been given a numeric quantity based on the characteristics of charge and hydrophobicity, called the hydropathic index (Kyte, J. and Doolittle, R. F. 1982), which can be used as a criterion for a substitution. The hydropathic index of the common amino acids are: Arg (−4.5); Lys (−3.9); Asn (−3.5); Asp (−3.5); Gln (−3.5); Glu (−3.5); His (−3.2); Pro (−1.6); Tyr (−1.3); Trp (−0.9); Ser (−0.8); Thr (−0.7); Gly (−0.4); Ala (+1.8); Met (+1.9); Cys (+2.5); Phe (+2.8); Leu (+3.8); Val (+4.2); and Ile (+4.5). Additionally, a value has also been given to various amino acids based on hydrophilicity, which can also be used as a criterion for substitution (U.S. Pat. No. 4,554,101). The hydrophilicity values for the common amino acids are: Trp (−3.4); Phe (−2.5); Tyr (−2.3); Ile (−1.8); Leu (−1.8); Val (−1.5); Met (−1.3); Cys (−1.0); Ala (−0.5); His (−0.5); Pro (−0.5+/−0.1); Thr (−0.4); Gly (0); Asn (+0.2); Gln (+0.2); Ser (+0.3); Asp (+3.0+/−0.1); Glu (+3.0+/−0.1); Arg (+3.0); and Lys (+3.0). In aspects wherein an amino acid is being conservatively substituted for an amino acid whose hydropathic index or hydrophilic value is similar, the difference between the respective index and/or value is preferably within +/−2, more preferably within +/−1, and most preferably within +/−0.5. In aspects wherein an amino acid is being non-conservatively substituted for an amino acid whose hydropathic index or hydrophilic value is similar, the difference between the respective index and/or value is preferably greater than +/−0.5, more preferably greater than +/−1, and most preferably greater than +/−2.

In certain embodiments, a functional equivalent may be produced by a non-mutation based chemical modification to an amino acid, a peptide or a polypeptide. Examples of chemical modifications include, when applicable, a hydroxylation of a proline or a lysine; a phosphorylation of a hydroxyl group of a serine and/or a threonine; a methylation of an alpha-amino group of a lysine, an arginine and/or a histidine (Creighton, T. E., 1983); adding a detectable label such as a fluorescein isothiocyanate compound (“FITC”) to a lysine side chain and/or a terminal amine (Rogers, K. R. et al., 1999); covalent attachment of a poly ethylene glycol (Yang, Z. et al., 1995; Kim, C. et al., 1999; Yang, Z. et al., 1996; Mijs, M. et al., 1994); an acylatylation of an amino acid, particularly at the N-terminus; an amination of an amino acid, particularly at the C-terminus (Greene, T. W. and Wuts, P. G. M. “Productive Groups in Organic Synthesis,” Second Edition, pp. 309-315, John Wiley & Sons, Inc., USA, 1991); a deamidation of an asparagine or a glutamine to an aspartic acid or glutamic acid, respectively; a derivation of an amino acid by a sugar moiety, a lipid, a phosphate, or a farnysyl group; an aggregation (e.g., a dimerization) of a plurality of proteinaceous molecules, whether of identical sequence or varying sequences; a cross-linking of a plurality of proteinaceous molecules using a cross-linking agent [e.g., a 1,1-bis(diazoacetyl)-2-phenylethane; a glutaraldehyde; a N-hydroxysuccinimide ester; a 3,3′-dithiobis (succinimidyl-propionate); a bis-N-maleimido-1,8-octane]; an ionization of an amino acid into an acidic, basic or neutral salt form; an oxidation of an amino acid; or a combination thereof of any of the forgoing. Such modifications may produce an alteration in a property of a proteinaceous molecule. For example, it is contemplated that a N-terminal glycosylation may enhance a proteinaceous molecule's stability (Powell, M. F. et al., 1993). In an additional example, it is contemplated that substitution of a beta-amino acid isoserine for a serine may enhance the aminopeptidase resistance a proteinaceous molecule (Coller, B. S. et al., 1993).

A proteinaceous molecule may comprise a proteinaceous molecule longer or shorter than the wild-type amino acid sequences. For example, an enzyme comprising longer or shorter sequences is encompassed, insofar as it retains enzymatic activity. In some embodiments, a proteinaceous molecule may comprise one or more peptide and/or polypeptide sequences. In certain embodiments, a modification to a proteinaceous molecule may add and/or subtract one or two amino acids from a peptide and/or polypeptide sequence. In other embodiments, a change to a proteinaceous molecule may add and/or remove one or more peptide and/or polypeptide sequences. Often a peptide or a polypeptide sequence may be added or removed to confer or remove a specific property from the proteinaceous molecule, and numerous examples of such modifications to a proteinaceous molecule are described herein, particularly in reference to fusion proteins. In particular, the native OPH of Pseudomonas diminuta is produced with a short amino acid sequence at its N-terminals that promotes the exportation of the protein through the cell membrane and is later cleaned. Thus, in certain embodiment, this signal sequence amino acid sequence is deleted by genetic modification in the DNA construction placed into Escherichia coli host cells to enhance its production.

As used herein, a “peptide” comprises a contiguous molecular sequence from 3 to 100 amino acids in length, including all intermediate ranges and combinations thereof. A sequence of a peptide may be 3 to 100 amino acids in length, including all intermediate ranges and combinations thereof. As used herein a “polypeptide” comprises a contiguous molecular sequence 101 amino acids or greater. Examples of a sequence length of a polypeptide include 101 to 10,000 amino acids, including all intermediate ranges and combinations thereof. As used herein a “protein” is a proteinaceous molecule comprising a contiguous molecular sequence three amino acids or greater in length, matching the length of a biologically produced proteinaceous molecule encoded by the genome of an organism.

It is recognized that removal of one or more amino acids from an enzyme's sequence may reduce or eliminate a detectableproperty such as enzymatic activity. However, it is further contemplated that a longer sequence, particularly a proteinaceous molecule may consecutively or non-consecutively comprises or even repeats one or more enzymatic sequences, including but not limited to those disclosed herein. Additionally, fusion proteins may be bioengineered to comprise a wild-type sequence and/or a functional equivalent of an enzyme sequence and an additional peptide or polypeptide sequence that confers a property and/or function.

a. Lipolytic Enzymes Functional Equivalents

Using recombinant DNA technology, wild-type and mutant forms of numerous lipolytic genes have been expressed in various cell types and expression systems, for further characterization and analysis, as well as large scale production of lipolytic enzymes for industrial or commercial use. Often signaling sequences are added, deleted or modified to redirect an expressed enzyme's targeting to extracellular secretion to allow rapid purification from cellular material, and additional sequences, particularly tags (e.g., a poly His tag) are added to aid in purification. In other cases, an enzyme is targeted to the cell surface or to intercellular expression. Codon optimization is often used to enhance yield of enzyme produced in a host cell. For example, mutations converting one or more residues of a protease cleavage site can enhance resistance to protease digestion. In one example, chymotrypsin cleavage site residues 149-156 identified in Pseudomonas glumae lipase can be converted into proline, arginine or other residues for enhance enzyme stability against protease inactivation.

To improve stability, particularly thermostability, it is contemplated that mutations may be made that mimic the differences between a thermophilic lipolytic enzyme and a psychrophilic or mesophilic lipolytic enzyme. Examples of such mutations to improve stability, particularly thermostability, would be ones that improve the hydrophobic core packaging (i.e., enhance the ratio of the residues' volume within the van der Waals distances to total residues' volume; reduce the total enzyme surface-to-volume ratio); increases the percentage of arginine as charged residues, as arginine forms stabilizing ion-pairs; mutating peptide bonds that are liable to spontaneous or chemical (i.e., asn-gln, asp-pro) breakage; replaces residues susceptible to oxidation, such as methionine (e.g., met with leu) and aromatic residues, particularly those on the surface; and make such changes isomorphic (e.g., by use of residues of similar size during substitution mutations) to prevent voids from being created [In “Engineering of/with Lipases” (F. Xavier Malcata., Ed.) pp. 193-197, 1996].

The X-ray crystal structures for various lipolytic enzymes (e.g., Rhizomucor miehei lipase, Humicola lanugnosa lipase, Penicillium camemberti lipase, Geotrichum candidum lipase, human pancreatic lipase, Fusarium solani cutinase, Psuedomonas glumae lipase, human nonpancreatic phospholipase A_{2,} Naja Naja atra phospholipase A₂) have been solved, allowing comparison of lipolytic enzymes' structures and identification residues involved in function [In “Advances in Protein Chemistry, Volume 45 Lipoproteins, Apolipoproteins, and Lipases.” (Anfinsen, C. B., Edsall, J. T., Richards, Frederic, R. M., Eisenberg, D. S., and Schumaker, V. N. Eds.) Academic Press, Inc., San Diego, Calif., pp. 1-152, 1994; “Lipases their Structure, Biochemistry and Application” (Paul Woolley and Steffen B. Peterson, Eds.), pp. 1-243-270, 337-354, 1994.]. For example, comparison of lipolytic enzymes has identified interfacial activation induced conformational changes in the lid structure of many enzymes producing increases in hydrophobic surface area of the enzyme and formation of an oxyanion transition state binding site (“oxyanion hole”) that promotes catalysis. In contrast, cutinase lacks a lid structure and has a preformed oxyanion hole, so it does not need interfacial activation for lipolytic activity (Martinez, C. et al., 1994; Nicolas, A. et al., 1996).

As would be known to those of skill in the art, the availability of these crystal structures and computer modeling of sequences onto existing crystal structures allows targeted mutations and alterations to be made to residues identified as belonging to regions of the enzyme with specific functions (e.g., surface residues for solubility or substrate interactions, active site binding residues, lid domain residues, etc.) For example, cutinase Arg196Glu and Arg17Glu surface residues mutations improved stability in lithium dodecylsulphate, by mutating the charged surface residues to ones that are similarly charged as the detergent's hydrophilic head group, reducing detergent binding that destabilizes the enzyme. Substrate preference can be changed by alterations to binding site residues or residues of domains near the binding site. For example, the preference for cutinase for 4-5C FAs esters was shifted to 7-8C FA esters by a binding site A85F mutation. In another example, a Phe139Trp mutation of the lid domain of Candida antartica lipase improved activity against tributyrine substrate 4-fold after comparison to the crystal structures of the more active lipases from Rhizomucor miehei and Humicola lanuginosa. In an additional example, enantioselectivity for Humicola lanuginosa lipase was increased for 1-heptyl 2-methyldcanoate and decreased for phenyl 2-methyldecanoate by mutation to alter the open-lid conformation's electrostatic stability (In “Engineering of/with Lipases” (F. Xavier Malcata., Ed.) pp. 197-202, 1996).

In a further example, Lipolase™ and Lipolase Ultra™ are industrial lipases produced by multiple mutations to improve enzyme properties of temperature stability, proteolytic cleavage resistance, oxidation resistance, detergent resistance, and pH optimization. These lipases are mutated forms of the lipase isolated from Humicola lanuginsa, where negatively charged residues on the lid domain were replaced with positive or hydrophobic residues (e.g., D96L) to reduce repulsion of negatively charged FAs or surfactants associated with lipids, resulting in a 4-5 fold or greater improvement in multicycle activity tests. Mutations at Savinase™ cleavage sites (e.g., residues 160-169 and 206-215) also improved resistance to proteolytic digestion. As an alternative to such rational design of mutations based on comparison of similar enzymes sequences, crystal structures, etc., bulk mutations via random mutation libraries may be used directed domain sequences implicated with stability or activity (e.g., lid domain in lipolytic enzymes, active site regions) to generate large numbers of mutants under selective screening protocols to mimic evolution and identify modified enzymes (In “Engineering of/with Lipases” (F. Xavier Malcata., Ed.) pp. 203-217, 1996).

Additional non-limiting examples of such recombinant expression of lipolytic enzymes, particularly enzymes having one or more mutations from the wild-type sequence (e.g., tags, signal sequences, mutations altering activity, etc.), know to those of skill in the art are shown on the table below.

TABLE 5
Examples of Recombinantly Expressed Lipolytic Enzymes

| Lipolytic Enzyme | Characteristics | Source/Host Cell | References |
|---|---|---|---|
| Carboxylesterase | lipA gene; preference for | Archaeoglobus fulgidus | Rusnak, M. et al., |
| | short chain FA ester; | DSM 4304/ | 2005. |
| | optimum activity 70° C., pH | Escherichia coli | |
| | 10-11 | | |
| Carboxylesterase | broad specificity, preference | Sulfolobus solfataricus | Park, Y. J. et al., |
| | for C8 FA esters; optimums | P1/Escherichia coli | 2006. |
| | 85° C., pH 8.0; detergent, urea | | |
| | and organic solvent resistant | | |
| Carboxylesterase | optimums 60° C., pH 7.5; Ca²⁺ | Thermotoga maritima | Kakugawa, S. et al., |
| | dependent | (tm0053)/Escherichia | 2007. |
| | | coli expressed as N- | |
| | | terminal hydrophobic | |
| | | region truncation | |
| Carboxylesterase | preference for C6 or less | Pseudomonas | Choi, G. S. et al., |
| | short chain FA esters | fluorescens/ | 2003. |
| | | Escherichia coli | |
| | | expression as a fusion | |
| | | protein with a N- | |
| | | terminal hexahistidine | |
| | | tag | |
| Carboxylesterase | active at 70° C., pH 7.1; some | Bacillus acidocaldarius/ | Manco, G. et al., |
| | enantioselectivity; strong | Escherichia coli | 1998. |
| | preference for short chain FA | | |
| | esters | | |
| Carboxylesterase | EstA gene | Burkholderia gladioli/ | Breinig, F. et al., |
| | | Saccharomyces | 2006. |
| | | cerevisiae, expressed as | |
| | | fusion protein on cell | |
| | | wall | |
| Carboxylesterase | preference for short chain FA | Pseudomonas | Pesaresi, A. et al., |
| | esters optimum activity 55° C., | aeruginosa PAO1/ | 2005. |
| | pH 9.0 | Escherichia coli | |
| Carboxylesterase | optimum activity pH 6.5-7.0; | Sulfolobus solfataricus | Morana, A. et al., |
| | preference for C2-C8 short | strain MT4/ | 2002. |
| | chain FA esters | Escherichia coli | |
| Carboxylesterase | estB gene; preference for C2-C6 | Burkholderia gladioli/ | Petersen, E. I. et al., |
| | short chain FA esters | Escherichia coli | 2001. |
| Carboxylesterase | EST2 gene; active at 70° C., | Archaeoglobus fulgidus/ | Manco, G. et al., |
| | pH 7.1 | Escherichia coli | 2000. |
| Carboxylesterase | lip8 gene; selective against | Pseudomonas | Ogino, H. et al., |
| | methyl and short chain FAs | aeruginosa LST- | 2004. |
| | esters | 03/Pseudomonas | |
| | | aeruginosa LST-03 | |
| Carboxylesterase | Thermoacidophilic | Sulfolobus shibatae/ | Huddleston, S. et al., |
| | | | 1995. |
| Carboxylesterase | stable at 90° C.; activity | Sulfolobus shibatae | Ejima, K. et al., |
| | against C2-C16 FA esters, | DSM5389/Escherichia | 2004. |
| | though not discernibly active | coli JM109 | |
| | against triacylglycerol | | |
| Carboxylesterase | Optimum activity 70° C.; | Alicyclobacillus | De Simone, G. et al., |
| | preference for 6C-8C FA | (formerly Bacillus) | 2000. |
| | esters | acidocaldarius/ | |
| | | Escherichia coli strain | |
| | | 834 (DE3) | |
| Carboxylesterase | active between 30° C.-90° C.; | Environment source | Rhee, J. K. et al., |
| | optimum activity pH 6.0, | library/Escherichia | 2005. |
| | good activity pH 5.5-7.5; | coli | |
| | preference for 10C or shorter | | |
| | FA esters | | |
| Carboxylesterase | estD gene; optimum activity | Thermotoga maritima/ | Levisson, M. et al., |
| | 95° C., pH 7; preference for | Escherichia coli | 2007. |
| | C4-C8 short chain FA esters | | |
| Carboxylesterase/ | Est3 gene; broad substrate | Sulfolobus solfataricus | Kim, S. and Lee, S. B., |
| Lipase | range - C2-C16; optimum | P2/Escherichia coli | 2004. |
| | 80° C., pH 7.4; some | | |
| | enantioselectivity | | |
| Carboxylesterase/ | p65 enzyme; preference for | Mycoplasma | Schmidt, J. A. et al., |
| Lipase | short chain fatty acids; | hyopneumoniae/ | 2004. |
| | optimums greater than 39° C., | Escherichia coli | |
| | pH 9.2-10.2 | expressed as glutathione | |
| | | S-transferase (GST)-p65 | |
| | | fusion protein after | |
| | | truncation of signal | |
| | | sequence | |
| Carboxylesterases/ | many isolates selective for | Fosmid and microbial | Lee, S. W. et al., |
| Lipases | short over long chain FA | DNA from forest | 2004. |
| | esters | topsoil/Escherichia coli | |
| | | secretion expression of | |
| | | 6 lipolytic enzymes with | |
| | | homology to hormone | |
| | | sensitive lipase and | |
| | | identified by library | |
| | | screening of tributyrin | |
| | | hydrolyzing isolates. | |
| Carboxylesterase/ | SSoNDelta and | Sulfolobus solfataricus/ | Mandrich, L. et al., |
| Lipases | SSoNDeltalong genes; | Escherichia coli strains | 2007. |
| | optimums pH 7.2, 70° C. and | Top10 and BL21(DE3) | |
| | pH 6.5, 85° C., respectively; | strains | |
| | both active against C4-C18 | | |
| | FA esters | | |
| Carboxylesterases/ | 3 enzymes expressed, | Myxococcus xanthus/ | Moraleda-Muñoz, A. |
| Lipases | preference for short chain FA | Escherichia coli BL21 | and Shimkets, L. J., |
| | esters | Star (DE3) expressed as | 2007. |
| | | lacZ fusion protein in | |
| | | (pET102/D-TOPO) | |
| | | vector system | |
| Carboxylesterase/ | Met(423)Ile, Met(423) Ile, | Rattus norvegicus/ | Wallace, T. J. et al., |
| Sterol esterase | Thr(444) Met mutations to | COS-7 expression of | 2001. |
| | mimic sequence of | mutant enzyme | |
| | cholesterol esterase in | | |
| | carboxylesterase conferred | | |
| | cholesterol esterase activity | | |
| Lipase | | Candida antarctica, A. oryzae | Tamalampudi, S. et |
| | | niaD300/ | al., 2007. |
| | | Aspergillus oryzae | |
| | | expressed in whole cells | |
| | | under improved glaA | |
| | | and pNo-8142 | |
| | | promoters and plasmids | |
| | | pNGA142 and | |
| | | pNAN8142, | |
| | | respectively, as fusion | |
| | | proteins with secretion | |
| | | signals and FLAG tags | |
| Lipase | Hepatic | Homo sapiens/rabbits | Rizzo, M. et al., |
| | | (transgenic) | 2004. |
| Lipase | | Geobacillus sp. strain | Rahman, R. N. et al., |
| | | T1/Escherichia coli | 2005. |
| | | Top10, TG1, XL1-Blue, | |
| | | BL21(De3)plysS, and | |
| | | Origami B, secretion | |
| | | expression via plasmid | |
| | | pGEX/T1S and pJL3 | |
| | | vectors | |
| Lipase | optimums 60-65° C., pH 9.0-10.0 | Bacillus | Kim, H. K. et al., |
| | | stearothermophilus L1/ | 1998. |
| | | Escherichia coli, Ala | |
| | | replaces the 1st Gly in | |
| | | the GlyXaaSerXaaGly | |
| | | sequence | |
| Lipase | bile salt stimulated | Homo sapiens/Pichia | Sahasrabudhe, A. V. |
| | | pastoris secretion | et al., 1998. |
| | | expression | |
| Lipase | optimum 68° C.; stability noted | Bacillus | Kim, M. H. et al., |
| | at 55° C.; stability increased | stearothermophilus L1/ | 2000. |
| | 8° C.⁺ by Ca²⁺. | Escherichia coli | |
| | | secretion expression via | |
| | | pET-22b(+) vector | |
| Lipase | stable at 60° C., pH 8.0; active | GeoBacillus | Abdel-Fattah, Y, R., |
| | at 100° C. | thermoleovorans Toshki/ | and Gaballa AA., |
| | | Escherichia coli via | 2008. |
| | | T7 promoter and pET | |
| | | 15b vector | |
| Lipase | bile salt stimulated | Homo sapiens/ | Downs, D. et al., |
| | | Escherichia coli via T7 | 1994. |
| | | expression system, N- | |
| | | terminus truncated. | |
| Lipase | | Homo sapiens (hepatic | Rashid, S. et al., |
| | | lipase)/rabbit | 2003. |
| | | transfected with | |
| | | adenovirus expressing | |
| | | lipase gene | |
| Lipase | alkaline lipase | Penicillium cyclopium | Wu, M. et al., 2003. |
| | | PG37/Escherichia coli | |
| | | expression in pET-30a | |
| Lipase | microsomal; S221A, E354A, | Homo sapiens/SF-9 | Alam, M. et al., |
| | and H468A mutants inactive; | cells secretion | 2002. |
| | N-glycosylation site N79A | expression | |
| | mutant not glycosylated; C- | | |
| | terminal endoplasmic | | |
| | reticulum retrieval signal | | |
| | deletion prevented secretion | | |
| Lipase | | Rhizopus oryzae/ | Washida, M. et al., |
| | | Saccharomyces | 2001. |
| | | cerevisiae expressed as | |
| | | a cell surface fusion | |
| | | protein of the pre-alpha- | |
| | | factor leader sequence | |
| | | and a C-terminal alpha- | |
| | | agglutinin segment | |
| | | including a | |
| | | glycosylphosphatidylinositol- | |
| | | anchor | |
| Lipase | bile salt-stimulated | Homo sapiens/Pichia | Murasugi, A. et al., |
| | | pastoris, expressed | 2001. |
| | | under AOX1 gene | |
| | | promoter, C-terminus | |
| | | truncated to enhance | |
| | | secretion | |
| Lipase | | Candida antarctica/ | Gustavsson, M. et |
| | | Pichia pastoris, | al., 2001. |
| | | expressed as a cellulose- | |
| | | binding domain fusion | |
| | | protein for | |
| | | immobilization onto | |
| | | cellulose | |
| Lipase | Thermostable | Bacillus | Sinchaikul, S. et al., |
| | | stearothermophilus P1/ | 2002. |
| | | Escherichia coli | |
| Lipase | CpLIP2 | Candida parapsilosis/ | Neugnot, V. et al., |
| | | Saccharomyces | 2002. |
| | | cerevisiae, including C- | |
| | | terminal histidine tag | |
| Lipase | L167V mutation increased | Burkholderia cepacia | Yang, J. et al., 2002. |
| | preference for short chain | KWI-56/in vitro | |
| | esters; F119A/L167M | expression with | |
| | mutation increased preference | Escherichia coli S30 | |
| | for long-chain ester | transcription/translation | |
| | | system | |
| Lipase | preference for C2-C4 short | Acinetobacter species | Han, S. J. et al., 2003. |
| | chain esters; able to | SY-01/Bacillus subtilis | |
| | hydrolyze a wide range of | 168 | |
| | esters and monoesters; | | |
| | optimum 50° C., pH 10; stable | | |
| | pH 9-11, optimum | | |
| Lipase | | Serratia marcescens/S. marcescens | Idei, A. et al., 2002. |
| | | via lipA | |
| | | gene in pUC19 | |
| | | coexpressed with an | |
| | | ATP-binding cassette | |
| | | (ABC) exporter to | |
| | | enhance secretion in a | |
| | | feed batch system | |
| Lipases | endothelial cell-derived, | Homo sapiens/Homo | Ishida, T. et al., |
| | several isoforms | sapiens tissue cells, | 2004. |
| | | including endothelial | |
| | | cells, secreted isoform | |
| | | active. | |
| Lipase | lip1 | Kurtzmanomyces sp. I- | Kakugawa, K. et al., |
| | | 11/Pichia pastoris | 2002. |
| Lipase | optimums 50° C., pH 7.0; | Acinetobacter | Dharmsthiti, S. et al., |
| | stable at 37° C.; stable in the | calcoaceticus LP009/ | 1998. |
| | presence of 0.1% Triton X- | Aeromonas sobria | |
| | 100, Tween-80 or Tween-20, | | |
| | enhanced by Fe³⁺ | | |
| Lipases | CdLIP1, CdLIP2 and | Candida deformans | Bigey, F. et al., |
| | CdLIP3, EMBL Accession | CBS 2071/ | 2003. |
| | Nos AJ428393, AJ428394 | Saccharomyces | |
| | and AJ428395 | cerevisiae | |
| Lipase | BTL2 gene; stable in the | Bacillus | Quyen, D. T. et al., |
| | presence of detergents and | thermocatenulatus/ | 2003. |
| | organic solvents | Pichia pastoris GS115 | |
| | | secreted enzyme | |
| Lipase | Thermoalkaophilic | Bacillus | Schlieben, N. H. et |
| | | thermocatenulatus/ | al., 2004. |
| | | Escherichia coli | |
| | | secretion expression of | |
| | | His-tagged enzyme for | |
| | | metal affinity | |
| | | chromatography | |
| | | purification | |
| Lipase | | Y. lipolytica/Yarrowia | Nicaud, J. M. et al., |
| | | lipolytica expression by | 2002. |
| | | the hp4d promoter in | |
| | | fed batch culture | |
| Lipase | | Bacillus subtilis/ | Sánchez, M. et al., |
| | | Escherichia coli, | 2002. |
| | | Saccharomyces | |
| | | cerevisiae and Bacillus | |
| | | subtilis via pBR322, | |
| | | YEplac112 and | |
| | | pUB110-derived | |
| | | vectors. | |
| Lipase | lipF gene, effective on short | Mycobacterium | Zhang, M. et al., |
| | chain FAs esters | tuberculosis/ | 2005. |
| | | Escherichia coli, | |
| | | expressed as fusion | |
| | | protein, site directed | |
| | | mutation of Ser90, | |
| | | Glu189, His219 active | |
| | | site residues. | |
| Lipase | | Oryza sativa/ | Kim, Y., 2004. |
| | | Escherichia coli | |
| | | expression by a pET | |
| | | expression system, | |
| | | enzyme associated with | |
| | | cell rather than secreted | |
| Lipases | ipla2epsilon, ipla2zeta, and | Homo sapiens/ | Jenkins, C. M. et al., |
| | ipla2eta | Spodoptera frugiperda | 2005. |
| | | SF9 cell | |
| Lipase | lipB52 gene; optimums: | Pseudomonas | Jiang, Z. et al., 2005. |
| | 40° C., pH 8.0 | fluorescens/Pichia | |
| | | pastoris KM71, secreted | |
| | | via pPIC9K vector | |
| | | expression | |
| Lipase | lip1 gene; thermostable | Candida rugosa/Pichia | Chang, S. W. et al., |
| | | after conversion of 19 | 2005. |
| | | CTG non-universal | |
| | | codons into universal | |
| | | codons to enhance | |
| | | enzyme production. | |
| Lipase | lip2 gene | Yarrowia lipolytica/ | Fickers, P. et al., |
| | | Yarrowia lipolytica | 2005. |
| | | strain LgX64.81 batch | |
| | | of fed batch | |
| | | extracellular expression | |
| Lipase | | Bacillus | Ahn, J. O. et al., |
| | | stearothermophilus L1/ | 2004. |
| | | Saccharomyces | |
| | | cerevisiae secreted | |
| | | under the galactose- | |
| | | inducible GAL10 | |
| | | promoter as a cellulose- | |
| | | binding domain fusion | |
| | | protein, the alpha- | |
| | | amylase signal peptide | |
| | | after fed batch | |
| | | production | |
| Lipase | | Rhizopus oryzae/ | Resina, D. et al., |
| | | Pichia pastoris | 2005. |
| | | expressed by FLD1 | |
| | | promoter in fed batch | |
| | | culture. | |
| Lipase | specificity for long chain | Lycopersicon | Matsui, K. et al., |
| | FAs; optimum pH 8.0 | esculentum L/ | 2004. |
| | | Escherichia coli | |
| | | SG13009 [pREP4], | |
| | | M15 [pREP4], Y1090, | |
| | | or Origami (DE3) | |
| | | strains used for | |
| | | intercellular expression | |
| Lipase | optimum 40° C., active up to | Geobacillus sp. | Li, H., Zhang X. et |
| | 90° C.; optimum pH 7.0-8.0, | TW1/Escherichia coli | al., 2005. |
| | pH range 6.0-9.0; stable in | as glutathione S- | |
| | 0.1% detergents Tween 20, | transferase fusion | |
| | Chaps, Triton X-100; | protein. | |
| | enhanced by Ca²⁺, Mg²⁺, | | |
| | Zn²⁺, Fe²⁺ or Fe³⁺; inhibited | | |
| | by Cu²⁺, Mn²⁺, and Li⁺ | | |
| Lipase | alip1 gene; optimums 30° C., | Arxula adeninivorans/ | Böer, E. et al., 2005. |
| | pH 7.5; selective toward | Arxula adeninivorans | |
| | medium chain FAs esters of | using strong TEF1 | |
| | 8-10 carbons over short and | promoter | |
| | long chain FAs esters | | |
| Lipase | lipJ02 gene and lipJ03 gene; | Environmental DNA/ | Jiang, Z. et al., 2006. |
| | optimums 30° C. and 35° C., | Pichia pastoris KM71 | |
| | respectively; function at pH | via pPIC9K vector | |
| | 8.0 | secretion expression. | |
| Lipase | activators, Ca²⁺, K⁺, and | Bacillus subtilis strain | Ma, J. et al., 2006. |
| | Mg²⁺, 7 mM sodium | IFFI10210/B. subtilis | |
| | taurocholate; inhibitors, Fe²⁺, | strain IFFI10210 via | |
| | Cu²⁺, and Co²⁺, 10 mM | pBSR2 plasmid | |
| | sodium taurocholate | expression | |
| Lipase | Calip4 gene, selective for | Candida albicans/ | Roustan, J. L. et al., |
| | unsaturated over saturated FA | Saccharomyces | 2005. |
| | | cerevisiae secretion via | |
| | | codon change from | |
| | | CUG serine codon into | |
| | | a universal codon. | |
| Lipase | glip1 gene | Arabidopsis thaliana/ | Oh, I. S. et al., 2005. |
| | | Escherichia coli, | |
| | | secretion expression via | |
| | | a pGEX6P-1 vector | |
| Lipase | | Geobacillus sp. strain | Rahman, R. N. et al., |
| | | T1/Escherichia coli | 2005. |
| | | Origami B strain | |
| | | secretion after | |
| | | recombinant plasmid | |
| | | pGEX/T1S and pJL3 | |
| | | vector expression. | |
| Lipase | lipA gene | Serratia marcescens | Kawai, E. et al., |
| | | 8000 mutated by N- | 2001. |
| | | methyl-N′-nitro-N- | |
| | | nitrosoguanidine into a | |
| | | high expression strain | |
| | | GE14, extracellular | |
| | | enzyme | |
| Lipase | | Candida rugosa/Pichia | Passolunghi, S. et al., |
| | | pastoris enzyme | 2003. |
| | | secretion in batch | |
| | | culture, also expressed | |
| | | as a green fluorescent | |
| | | fusion protein to tract | |
| | | extracellular secretion | |
| | | pathway. | |
| Lipase | Ala substituted for the 1st Gly | Geobacillus sp. strain | Leow, T. C. et al., |
| | of the GlyXaaSerXaaGly | T1/E. coli intercellular | 2004. |
| | substrate binding site; | expression under | |
| | optimums 65° C., pH 9.0; | araBAD, T7, T7 lac, | |
| | active range pH 6-11 | and tac promoters in | |
| | | pBAD, pRSET, pET, | |
| | | and pGEX expression | |
| | | vectors. | |
| Lipase | | Bacillus subtilis/ | Narita, J. et al., 2006. |
| | | Escherichia coli via cell | |
| | | surface expression as a | |
| | | FLAG peptide-fusion | |
| | | protein | |
| Lipase | chimeric enzyme of 3 lipases; | Candida antarctica | Suen, W. C. et al., |
| | active at 45° C., a higher | ATCC 32657 + | 2004. |
| | temperature than parent | Hyphozyma sp. CBS | |
| | enzymes | 648.91 + Crytococcus | |
| | | tsukubaensis ATCC | |
| | | 24555/Saccharomyces | |
| | | cerevisiae | |
| Lipase | tglA gene | Aspergillus oryzae | Kaieda, M. et al., |
| | | niaD300/Aspergillus | 2004. |
| | | oryzae expression under | |
| | | a glaA promoter of | |
| | | plasmid pNGA142, | |
| | | whole-cells | |
| | | immobilized to | |
| | | biomass-support | |
| | | particles. | |
| Lipase | Ca²⁺-dependent, Mn²⁺ and | Pseudomonas sp./ | Rashid, N. et al., |
| | Sr²⁺ also enhances activity; | Escherichia coli | 2001. |
| | preference for C10 FAs and | | |
| | 1, 3 esters; optimum 35° C. | | |
| Lipase | | Thermomyces | Prathumpai, W. et |
| | | lanuginosus/ | al., 2004. |
| | | Aspergillus niger (strain | |
| | | NW 297-14 and | |
| | | NW297-24) expressed | |
| | | with Aspergillus oryzae | |
| | | TAKA amylase | |
| | | promoter, bound to cell | |
| | | wall after production | |
| Lipase | lipA gene | Pseudomonas | Kojima, Y., et al., |
| | | fluorescens HU380/ | 2003. |
| | | Escherichia coli, | |
| | | refolded from inclusion | |
| | | bodies | |
| Lipase | Liver lysosomal acid lipase | Homo sapiens/ | Zschenker, O. et al., |
| | | Spodoptera frugiperda | 2004. |
| | | insect cells by | |
| | | expression without the | |
| | | signal peptide sequence; | |
| | | mutation G50A inhibit | |
| | | activity possibly by | |
| | | preventing cleavage of | |
| | | preprotein | |
| Lipase | | Phlebotomus papatasi/ | Belardinelli, M. et |
| | | Escherichia coli via | al., 2005. |
| | | pQE30 vector | |
| | | expression. | |
| Lipase | active at 65° C. when absorbed | Bacillus | Palomo, J. M. et al., |
| | onto hydrophobic support | thermocatenulatus | 2004. |
| | | (BTL2)/Escherichia | |
| | | coli expressed, secreted | |
| | | enzyme absorbed onto | |
| | | hydrophobic support | |
| | | (octadecyl-Sepabeads) | |
| | | increased | |
| | | thermostability 10° C. | |
| Lipase | | Rhizopus oryzae/ | Resina, D. et al., |
| | | Pichia pastoris | 2004. |
| | | secretion expression | |
| | | under the formaldehyde | |
| | | dehydrogenase 1 | |
| | | promoter | |
| Lipase | | Homo sapiens | Broedl, U. C. et al., |
| | | (endothelial)/ | 2004. |
| | | transgenic mice | |
| Lipase | | Candida parapsilosis/ | Brunel, L. et al., |
| | | Pichia pastoris feed | 2004. |
| | | batch secretion | |
| | | expression by a | |
| | | methanol inducible | |
| | | alcohol oxidase 1 gene | |
| Lipase | | Homo sapiens (bile salt- | Trimble, R. B. et al., |
| | | stimulated lipase)/ | 2004. |
| | | Pichia pastoris secreted | |
| | | as glycoprotein | |
| Lipase | optimums pH 8.0, 29° C.; | Pseudomonas fragi | Alquati, C. et al., |
| | active at 10° C. and 50° C.; 3D | strain IFO 3458/ | 2002. |
| | computer modeling against | Escherichia coli | |
| | other lipases verified catalytic | SG13009 intercellular | |
| | triad: S83, D238 and H260, | expression | |
| | and oxyanion hole: L17, Q84 | | |
| Lipase | TliA gene | Pseudomonas | Song, J. K. et al., |
| | | fluorescens/Serratia | 2007. |
| | | marcescen coexpression | |
| | | of cognate ABC | |
| | | transporter improved | |
| | | production/secretion | |
| | | using pTliDEFA-223 | |
| | | plasmid. | |
| Lipase | lipI gene | Galactomyces | Fernández, L. et al., |
| | | geotrichum BT107/ | 2006. |
| | | Pichia pastoris | |
| | | secretion expression | |
| Lipase | optimums 40° C., pH 7.0-8.0; | Geobacillus sp. TW1/ | Li, H., and Zhang X., |
| | active up to 90° C. at pH 7.5; | Escherichia coli | 2005. |
| | stable at pH 6.0-9.0; stable in | expression as a | |
| | 0.1% detergents Tween 20, | glutathione S- | |
| | Chaps or Triton X-100; | transferase fusion | |
| | activity enhanced by Ca²⁺, | protein | |
| | Mg²⁺, Zn²⁺, Fe²⁺ or Fe³⁺, | | |
| | inhibited by Cu²⁺, Mn²⁺, or | | |
| | Li⁺ | | |
| Lipase | Gastric | Canis domesticus/corn | Zhong, Q. et al., |
| | | transgenic expression | 2006. |
| Lipase | BTL2 gene | Bacillus | Rua, M. L. et al., |
| | | thermocatenulatus/ | 1998. |
| | | Escherichia colicellular | |
| | | expression as fusion | |
| | | protein with OmpA | |
| | | outermembrane signal | |
| | | peptide in pCYT-EXP1 | |
| | | (pT1) expression vector | |
| Lipase | hybrid protein lost | Staphylococcus aureus | Nikoleit, K. et al., |
| | phospholipase activity but | NCTC8530 + | 1995. |
| | retained Ca²⁺ stimulation | Staphylococcus hyicus/ | |
| | relative to S. hyicus enzyme | Staphylococcus | |
| | | carnosus, secretion | |
| | | expression of a hybrid | |
| | | lipase having S. hyicus | |
| | | 146 residues) | |
| Lipase | lipCE gene; optimum 30° C. | Environmental source | Elend, C. et al., |
| | and pH 7.0; active at −5° C.; | isolation/Escherichia | 2007. |
| | preference for C10 FA ester, | coli, refolded from | |
| | but large range of substrates; | inclusion bodies | |
| | steriospecific for (R)- | | |
| | ibuprofen esters | | |
| Lipase | optimum 75° C. | Bacillus | Cho, A. R. et al., |
| | | thermoleovorans ID-1/ | 2000. |
| | | Escherichia coli | |
| | | expression via T7 | |
| | | promoter in pET-22b(+) | |
| | | vector | |
| Lipase | blie salt inhibited | Homo sapiens/Pichia | Sebban-Kreuzer, C. |
| | | pastoris secretion | et al., 2006. |
| | | expression via a | |
| | | pPIC9K vector | |
| Lipase | | Rhizopus oryzae/ | Resina, D. et al., |
| | | Pichia pastoris | 2007. |
| | | expression under the | |
| | | formaldehyde | |
| | | dehydrogenase | |
| | | promoter in fed-batch | |
| | | cultivation | |
| Lipase | | Thermomyces | Haack, M. B. et al., |
| | | lanuginosus/ | 2007. |
| | | Aspergillus oryzae | |
| | | expression in batch and | |
| | | fed-batch cultivation | |
| Lipase | | Aspergillus niger F044/ | Shu, Z. Y. et al., |
| | | Escherichia coli | 2007. |
| | | BL21(De3), refolded for | |
| | | activity after expression | |
| Lipase | Lysosomal acid | Homo sapiens/Homo | Pariyarath, R. et al., |
| | | sapiens HeLa cells | 1996. |
| | | expression via vaccinia | |
| | | T7 system | |
| Lipase | Hepatic | Homo sapiens/mice | Dugi, K. A. et al., |
| | | transgenic expression | 1997. |
| Lipase | | Candida rugosa/Pichia | Chang, S. W. et al., |
| | | pastoris, expression of a | 2006^{A}. |
| | | N-terminal peptide | |
| | | truncated with 18 non- | |
| | | universal CTG codons | |
| | | converted to TCT | |
| | | improved expression | |
| | | 52-fold | |
| Lipase | CtLIP gene; preference for 2- | Candida thermophila/ | Thongekkaew, J., |
| | position esters, optimum | Saccharomyces | Boonchird C., 2007. |
| | 55° C. | cerevisiae and Pichia | |
| | | pastoris as secreted | |
| | | enzyme under the | |
| | | alcohol oxidase gene | |
| | | (AOX1) promoter | |
| Lipase | active against broad range of | Staphylococcus | Sayari, A. et al., |
| | FA chain lengths; Asp290Ala | simulans/Escherichia | 2007. |
| | mutant preference for short | coli BL21 (DE3) | |
| | FA esters | expressed using a pET- | |
| | | 14b vector as a His- | |
| | | tagged enzyme | |
| Lipases | LIPY7 and LIPY8 genes | Yarrowia lipolytica/ | Jiang, Z. B. et al., |
| | | Pichia pastoris KM71 | 2007. |
| | | cell surface expression | |
| | | as fusion protein with | |
| | | Saccharomyces | |
| | | cerevisiae FLO- | |
| | | flocculation domain | |
| | | sequence, use of whole | |
| | | cell biocatalyst or | |
| | | cleaved enzyme | |
| Lipase | lipC gene | Bacillus subtilis ycsK/ | Masayama, A. et al., |
| | | Escherichia coli | 2007. |
| Lipase | optimums 55° C., pH 8.5; | Bacillus | Sinchaikul, S. et al., |
| | stable 30-65° C.; stable in | stearothermophlius P1/ | 2001. |
| | detergents 0.1% Chaps or | Escherichia coli | |
| | Triton X-100 | M15[EP4]; additional | |
| | | expression of site | |
| | | directed Ser-113, Asp- | |
| | | 317, and His-358 | |
| | | mutants confirmed | |
| | | active site residues | |
| Lipase | Asp290Ala mutant had | Staphylococcus xylosus/ | Mosbah, H. et al., |
| | altered FA chain length | Escherichia coli BL21 | 2006. |
| | specificity | (DE3) using pET-14b | |
| | | vector, strong T7 | |
| | | promoter, and 6 N- | |
| | | terminal histidines | |
| Lipase | LIP4 mutations A296I, | Candida rugosa/Pichia | Lee, L. C. et al., |
| | V344Q, and V344H | pastoris | 2007. |
| | improved activity against | | |
| | short chain FA esters; A296I | | |
| | and V344Q mutations | | |
| | improved activity toward | | |
| | medium and long chain FA | | |
| | esters | | |
| Lipase | preference for C16-C18 long | Candida rugosa/Pichia | Tang, S. J. et al., |
| | chain FA esters; stable at | pastoris and | 2001. |
| | 58° C. when glycosylated in P. pastoris | Escherichia coli | |
| | expression; 52° C. | expression improved by | |
| | unglycosylated in | mutation of 19 non- | |
| | Escherichia coli expression; | universal CUG codons | |
| | no interfacial activation | into universal codons. | |
| Lipase | Phe94Gly mutant has | Rhizomucor miehei/ | Gaskin, D. J. et al., |
| | increased preference for short | Escherichia coli | 2001. |
| | chain FA esters | expression of mutants | |
| Lipase | broad substrate specificity, | Bacillus licheniformis/ | Nthangeni, M. B. et |
| | but preference for C6-C8 FA | Escherichia coli | al., 2001. |
| | esters | expression a secreted | |
| | | fusion protein with 6 C- | |
| | | terminal histidines. | |
| Lipase | Lysosomal acid | Homo sapiens/ | Ikeda, S. et al., 2004. |
| | | Schizosaccharomyces | |
| | | pombes as secreted | |
| | | protein via feed batch | |
| | | growth | |
| Lipase | Gly311Val mutant stable at | Staphylococcus xylosus/ | Mosbah, H. et al., |
| | 50° C.; G311D mutant | Escherichia coli BL21 | 2007. |
| | optimum pH 6.5; G311K | (DE3) | |
| | mutant optimum pH 9.5 | | |
| Lipase | F417A mutation in neutral | Homo sapiens/ | Alam, M. et al., |
| | lipid binding domain | Spodoptera frugiperda | 2006. |
| | FLXLXXXn reduces ester | SF9 cells | |
| | hydrolysis rate | | |
| Lipase | | Rhizopus oryzae/ | Di Lorenzo, M. et |
| | | Escherichia coli | al., 2005. |
| | | Origami(DE3) using | |
| | | pET-11d vector | |
| | | expression. | |
| Lipase | LIP1 gene | Candida rugosa/Pichia | Chang, S. W. et al., |
| | | pastoris | 2006^{B}. |
| Lipase | optimums 40° C., pH 5.8 | Malassezia furfur/ | Brunke, S., and |
| | | Pichia pastoris | Hube B. et al., 2006. |
| Lipase | optimums 60-70° C., pH 8.0-9.0; | Bacillus | Schmidt-Dannert, C. |
| | stable at pH 9.0-11.0; | thermocatenulatus./ | et al., 1996. |
| | stable in contact with | Escherichia coli | |
| | detergents and organic | DH5alpha expression | |
| | solvents | via pUC18 vector, Ala | |
| | | replaces 1st Gly of Gly- | |
| | | X-Ser-X-Gly consensus | |
| | | sequence | |
| Lipase | OST gene; 1, 3 position | Bacillus sphaericus | Sulong, M. R. et al., |
| | specificity; organic solvent | 205y/Escherichia coli | 2006. |
| | tolerance; optimums 55° C., | | |
| | pH 7.0-8.0; range 5.0-13.0 at | | |
| | 37° C.; activity enhance by | | |
| | Ca²⁺, Mg²⁺, | | |
| | dimethylsulfoxide (DMSO), | | |
| | methanol, p-xylene and n- | | |
| | decane | | |
| Lipase | lipB68 gene; optimum 20° C.; | Pseudomonas | Luo, Y. et al., 2006. |
| | 1, 3 FA ester preference | fluorescens strain B68/ | |
| Lipases | LIPY7 and LIPY8 genes | Yarrowia lipolytica/ | Song, H. T. et al., |
| | | Pichia pastoris KM71 | 2006. |
| | | secreted expression in | |
| | | the expression vector | |
| | | pPIC9K with 6 x | |
| | | Histidine tag sequence | |
| Lipase | Lip9 gene, stable in contact | Pseudomonas | Ogino, H. et al., |
| | with organic solvents | aeruginosa LST-03/ | 2007. |
| | | Escherichia coli | |
| | | coexpression with | |
| | | lipase-specific foldase | |
| | | (Lif9), T7 promoter | |
| | | used, lipase signal | |
| | | peptide deleted, | |
| | | overexpression | |
| | | inclusion bodies | |
| | | refolded | |
| Lipases | lipase A and lipase B | Bacillus subtilis/ | Detry, J. et al., 2006. |
| | | Escherichia coli | |
| | | purified or crude cell | |
| | | lyophilizate | |
| | | preparations by batch | |
| | | and repetitive batch | |
| | | growth. | |
| Lipase | YlLip2 gene; optimums 40° C., | Yarrowia lipolytica/ | Yu, M et al., 2007. |
| | pH 8.0; preference for C12-C16 | Pichia pastoris X-33, | |
| | long chain FA esters | secretion expression as | |
| | | fusion protein with | |
| | | Saccharomyces | |
| | | cerevisiae secretion | |
| | | signal peptide, under | |
| | | methanol inducible | |
| | | promoter of the alcohol | |
| | | oxidase 1 gene in | |
| | | pPICZalphaA vector, | |
| | | fed batch growth | |
| Lipase | | Candida rugosa/Pichia | Chang, S. W. et al., |
| | | pastoris expression | 2006^{C}. |
| | | increased over 4 fold by | |
| | | mutating codons into P. pastoris | |
| | | preferred | |
| | | codons | |
| Lipase/ | vst gene; preference for C12 | Vibrio harveyi strain | Teo, J. W. et al., |
| Carboxylesterase | long chain FA esters, able to | AP6/Escherichia coli | 2003. |
| | hydrolyze short, medium and | TOP10 cell expression | |
| | longer chain FA esters | as a carboxy-terminal 6 | |
| | | x His tagged enzyme | |
| Lipase/ | broad specificity for 2C-18C | Oil-degrading | Mizuguchi, S. et al., |
| Carboxylesterase | FA esters | bacterium, strain HD-1/ | 1999. |
| | | Escherichia coli | |
| Lipases/ | multiple isolates | Lipase/esterase libraries/ | Ahn, J. M. et al., |
| Carboxylesterases | | Escherichia coli | 2004. |
| | | secretion expression | |
| Lipase/ | S-enantioselective; preference | Yarrowia lipolytica | Kim, J. T. et al., |
| Carboxylesterase | for <=10C FA esters; | CL180/Escherichia | 2007. |
| | optimum pH 7.5, 35° C. | coli | |
| Co-lipase | | Homo sapiens/Pichia | D'Silva, S. et al., |
| | | pastoris | 2007. |
| Phospholipase/ | selective for phospholipids | Arabidopsis rosette/ | Lo, M. et al., 2004. |
| Lipase | | Escherichia coli | |
| Lipases/Cutinase | Bacillus subtilis and Serratia | Bacillus subtilis, | Becker, S. et al., |
| | marcescens lipases, and | Fusarium solani pisi, | 2005. |
| | cutinase from Fusarium | Serratia marcescens/ | |
| | solani pisi | Escherichia coli | |
| | | expressed lipolytic on | |
| | | cell surface as a | |
| | | membrane anchored | |
| | | fusion proteins | |
| Lipoprotein lipase | | Homo sapiens/rabbits | Fan, J. et al., 2001. |
| | | (transgenic) | |
| Lipoprotein lipase | multiple mutations to alter | Avian/Chinese hamster | Sendak, R. A., and |
| | protein surface charge mildly | ovary cells expression, | Bensadoun A. J, |
| | reduced activity | multiple site-directed | 1998. |
| | | mutations Lys 321, Arg | |
| | | 405, Arg 407, Lys 409, | |
| | | Lys 415, and Lys 416 | |
| | | for alter heparin- | |
| | | Sepharose binding | |
| Lipoprotein lipase | | Homo sapiens/insect | Zhang, L. et al., |
| | | cells (sf21) | 2003. |
| Acylglycerol | | Mus musculus/African | Karlsson, M. et al., |
| lipase | | green monkey COS | 1997. |
| | | cells | |
| Acylglycerol | | Mus musculus/Sf9 | Karlsson, M. et al., |
| lipase | | cells via an baculovirus- | 2000. |
| | | insect expression | |
| | | system | |
| Acylglycerol | diacylglycerol lipase activity | Penicillium camembertii | Yamaguchi, S. et al., |
| lipase | | U-150/Aspergillus | 1997. |
| | | oryzae, expressed using | |
| | | own promoter | |
| Acylglycerol | | Bacillus sp. strain H- | Kitaura, S. et al., |
| lipase | | 257/Escherichia coli | 2001. |
| | | via a pACYC184 | |
| | | plasmid vector | |
| Acylglycerol | Rv0183 gene; preference for | Mycobacterium | Côtes, K. et al., |
| lipase | monoacylglycerol over di- or | tuberculosis/ | 2007. |
| | triacylglycerol; optimum pH | Escherichia coli | |
| | 7.7-9.0 | | |
| Acylglycerol | | Homo sapiens/mice | Coulthard, M. G. et |
| lipase | | expression via | al., 1996. |
| | | adenovirus vector | |
| Acylglycerol | rHPLRP2 gene, active pH 5-7+ | Homo sapiens/Pichia | Eydoux, C. et al., |
| lipase/ | range | pastoris secreted | 2007. |
| Galactolipase | | | |
| Phospholipase/ | patatin protein has multi- | Solanum tuberosum/ | Andrews, D. L. et al., |
| Acylglycerol | enzyme activity; strong | Spodoptera frugiperda | 1988. |
| lipase/ | preference for | SF9 cells | |
| Galactolipase | monacylglycerol over di- or | | |
| | tri-acylglycerols | | |
| Hormone Sensitive | | Homo sapiens/ | Contreras, J. A. et al., |
| Lipase | | Spodoptera frugiperda | 1998. |
| | | SF9 cells | |
| Hormone Sensitive | | Mus musculus/THP-1 | Okazaki, H. et al., |
| Lipase | | macrophage-like cells | 2002. |
| | | by adenovirus-mediated | |
| | | gene delivery | |
| Hormone Sensitive | | Rattus norvegicus/ | Kraemer, F. B. et al., |
| Lipase/Sterol | | Escherichia coli | 1993. |
| esterase | | expression of truncated | |
| | | enzyme fusion protein | |
| | | via a pET expression | |
| | | system | |
| Phospholipase A₁ | | Serratia sp. MK1/ | Song, J. K et al., |
| | | Escherichia coli, | 1999. |
| | | expression improved by | |
| | | promoter with lower | |
| | | strength, lower | |
| | | temperature, enriched | |
| | | medium. | |
| Phospholipase A₁ | | Aspergillus oryzae/ | Shiba, Y. et al., |
| | | Saccharomyces | 2001. |
| | | cerevisiae and A. oryzae | |
| Phospholipase A₁ | mPAPLA1alpha and | Homo sapiens (testes)/ | Hiramatsu, T. et al., |
| | mPAPLA1beta, selective for | Homo sapiens HeLa | 2003. |
| | phosphatidic acid | cells secretion | |
| | | expression for mPA- | |
| | | PLA1alpha, cell | |
| | | membrane association | |
| | | for mPA-PLA1beta | |
| Phospholipase A₁ | dad1 | Arabidopsis/ | Ishiguro, S. et al., |
| | | Escherichia coli and in | 2001. |
| | | Arabidopsis as a fusion | |
| | | with green fluorescent | |
| | | protein | |
| Phospholipase A₂ | optimum pH 8-10 | Nicotiana tabacum/ | Fujikawa, R. et al., |
| | | Escherichia coli | 2005. |
| | | expression as a | |
| | | thioredoxin fusion | |
| | | protein within cells | |
| Phospholipase A₂ | cytosolic; cPLA₂delta, | Mus musculus/Homo | Ohto, T. et al., 2005. |
| | cPLA₂epsilon and cPLA₂zeta | sapiens embryonic | |
| | genes; Ca²⁺ dependant | kidney 293 cells | |
| | activity | | |
| Phospholipase A₂ | plaA gene; substrates PC and | Aspergillus nidulans/ | Hong, S. et al., 2005. |
| | PE | yeast cells expression of | |
| | | N-truncated enzyme | |
| Phospholipase A₂ | Lipoprotein-associated | Homo sapiens/Pichia | Zhang, F et al., 2006. |
| | | pastoris secretion | |
| | | expression | |
| Phospholipase A₂ | Ca²⁺ activated | Arabidopsis thaliana/ | Mansfeld, J. et al., |
| | | Escherichia coli | 2006. |
| Phospholipase A₂ | Ca⁺² dependent, optimum pH | Drosophila | Ryu, Y. et al., 2003. |
| | 5.0 | melanogaster/ | |
| | | Escherichia coli | |
| Phospholipase A₂ | 3 isoforms expressed | Naja naja sputatrix/ | Armugam, A. et al., |
| | | Escherichia coli | 1997. |
| Phospholipase A₂ | Calcium independent, | Mus musculus, Bos | Hiraoka, M. et al., |
| | AXSXG catalytic site | taurus, and Homo | 2002. |
| | sequence. | sapiens (kidney)/COS- | |
| | | 7 cells via pcDNA3 | |
| | | vector, producing | |
| | | carboxyl-terminally | |
| | | tagged proteins | |
| Phospholipase A₂/ | optimum 90° C. | Aeropyrum pernix K1 | Wang, B. et al., |
| Carboxylesterase | | APE2325/Escherichia | 2004. |
| | | coli BL21 (DE3) Codon | |
| | | Plus-RIL | |
| Phospholipase B | | Guinea pig/Monkey | Nauze, M. et al.,” |
| | | Kidney COS-7 cells | 2002. |
| | | expressed including | |
| | | mutants identifying | |
| | | serine 399 as | |
| | | functioning in activity | |
| | | and truncation mutants. | |
| Phospholipase C | active at 70° C. +, pH 3.5-6.0 | Bacillus cereus/ | Durban, M. A. et al., |
| | | Bacillus subtilis | 2007. |
| | | expression via a | |
| | | acetoin-controlled | |
| | | expression system | |
| Phospholipase C | phosphatidylinositol-specific | Bacillus thuringiensis/ | Kobayashi, T. et al., |
| | | Bacillus brevis 47 | 1996. |
| | | expression system | |
| Phospholipase C | broad specificity for | Bacillus cereus/ | Tan, C. A. et al., |
| | phospholipids | Escherichia coli via a | 1997. |
| | | T7 expression system, | |
| | | refolded form inclusion | |
| | | bodies | |
| Phospholipase C | phosphoinositide-specific | Zea mays/Escherichia | Zhai, S. et al., 2005. |
| | | coli | |
| Phospholipase C | plc gene; stable at 75° C., | Bacillus cereus/Pichia | Seo, K. H., Rhee JI., |
| | optimum pH 4.0-5.0 | pastoris secretion | 2004. |
| | | expression as a alpha- | |
| | | factor secretion signal | |
| | | peptide fusion protein | |
| Phospholipases C | Phosphoinositide-specific | Pisum sativum/ | Venkataraman, G. et |
| | | Escherichia coli | al., 2003. |
| Phosphatidate | Mg²⁺⁻independent, lyso-PA | Saccharomyces | Toke, D. A. et al., |
| phosphatase | phosphatase and | cerevisiae/Sf-9 insect | 1998. |
| | diacylglycerol pyrophosphate | cells | |
| | phosphatase activity | | |
| Lysophospholipase | | Clonorchis sinensis/ | Ma, C. et al., 2007. |
| | | Escherichia coli | |
| Sterol esterase | | Homo sapiens/COS-7 | Zhao, B. et al., 2005. |
| | | cell expression | |
| Sterol esterase | hncCEH gene, hepatic | Rattus norvegicus/ | Langston, T. B. et al., |
| | | mice infected with | 2005. |
| | | AdCEH adenovirus | |
| | | vector under Homo | |
| | | sapiens | |
| | | cytomegalovirus | |
| | | promoter, liver cell | |
| | | enzyme expression | |
| | | evaluated | |
| Sterol esterase | | Rattus norvegicus/ | DiPersio, L. P. et al., |
| | | Spodoptera frugiperda | 1992. |
| | | (Sf9) insect cells | |
| | | secretion expression via | |
| | | a Baculovirus transfer | |
| | | vector pVL1392 | |
| Sterol esterase | | Homo sapiens/COS-1 | Ghosh, S., 2000. |
| | | and COS-7 cells | |
| | | expression via | |
| | | expression vector, | |
| | | pcDNA3.1/V5/His- | |
| | | TOPO, | |
| Sterol esterase | CLR1, CRL3 and CRL4 | Candida rugosa/Pichia | Brocca, S. et al., |
| | isozymes used to make | pastoris X33 expression | 2003. |
| | hybrid enzymes by switching | of hybrid protein under | |
| | lid sequence into CLR1, | the he methanol- | |
| | conferring cholesterol | inducible alcohol | |
| | esterase activity and detergent | oxidase promoter | |
| | sensitivity, but no change in | | |
| | chain length preference | | |
| Sterol esterase | | Rattus norvegicus/Hep | Hall, E. et al., 2001. |
| | | G2 cells and Chinese | |
| | | hamster ovary cells via | |
| | | a replication-defective | |
| | | recombinant adenovirus | |
| | | vector | |
| Sterol esterase | ste1 | Melanocarpus | Kontkanen, H. et al., |
| | | albomyces/Pichia | 2006. |
| | | pastoris and T. reesei | |
| | | under inducible AOX1 | |
| | | promoter, under the | |
| | | inducible cbh1 | |
| | | promoter, respectively | |
| Galactolipase | Vupat1 gene; active on | Vigna unguiculata/ | Matos, A. R. et al., |
| | monogalactosyldiacylglycerol, | Spodoptera frugiperda | 2000. |
| | digalactosyldiacylglycerol | SF9 cells | |
| | and | | |
| | sulphoquinovosyldiacylglycerol | | |
| Galactolipase | | Homo sapiens/Pichia | Sias, B. et al., 2004. |
| | | pastoris and insect cells | |
| Galactolipase | | Homo sapiens/Pichia | Sias, B. et al., 2004. |
| | | pastoris and insect cells | |
| Sphingomyelin | | Bacillus cereus/ | Tamura, H. et al., |
| phosphodiesterase | | Bacillus brevis 47 | 1992. |
| | | expression as a cell wall | |
| | | signal sequence fusion | |
| | | protein U211 vector | |
| Sphingomyelin | | Homo sapiens/ | Lee, C. Y. et al., |
| phosphodiesterase | | secretion expression in | 2007. |
| | | Chinese hamster ovary | |
| | | cells, N-terminal | |
| | | truncations prevented | |
| | | secretion and enzyme | |
| | | activity | |
| Sphingomyelin | | Homo sapiens/COS-7 | Wu, J. et al., 2005. |
| phosphodiesterase | | cell expression of | |
| | | glycosylation mutants | |
| | | demonstrated less | |
| | | activity | |
| Sphingomyelin | | Bacillus cereus/ | Nishiwaki, H. et al., |
| phosphodiesterase | | Escherichia coli, | 2004. |
| | | His151Ala mutant | |
| | | inactive | |
| Sphingomyelin | Sphingomyelin-specific | Pseudomonas sp. strain | Sueyoshi, N. et al., |
| phosphodiesterase | sphingomyelinase C; able to | TK4/Escherichia coli | 2002. |
| | hydrolyze short FA ester | Dhalpha and | |
| | chain containing | BL21(DE3)pLysS | |
| | sphingomyelin; optimum pH | | |
| | 8.0, activated by Mn²⁺ | | |
| Phospholipase D | | Homo sapiens/COS-7 | Lehman, N. et al., |
| | | cells with a myc- | 2007. |
| | | (pcDNA)-PLD2 vector | |
| Phospholipase D | | Arabidopsis thaliana/ | Qin, C. et al., 2006. |
| | | Escherichia coli | |
| Phospholipase D | | Streptoverticillium | Ogino, C. et al., |
| | | cinnamoneum/ | 2004. |
| | | Streptomyces lividans | |
| | | via an Escherichia coli | |
| | | shuttle vector-pUC702 | |
| Phospholipase D | | Homo sapiens/COS7 | Di Fulvio, M. et al., |
| | | cells | 2007. |
| Phospholipase D | | Vigna unguiculata L. Walp/ | Ben, Ali Y. et al., |
| | | Pichia pastoris | 2007. |
| | | secretion expression | |
| Ceramidase | | Pseudomonas | Nieuwenhuizen, W. F. |
| | | aeruginosa PA01/ | et al., 2003. |
| | | Escherichia coli | |
| | | DH5alpha intracellular | |
| | | expression under lac- | |
| | | promoter, Escherichia | |
| | | coli BL21 intracellular | |
| | | expression under T7- | |
| | | promoter forming | |
| | | refoldable inclusion | |
| | | bodies without signal, | |
| | | Pseudomonas putida | |
| | | extracellular expression | |
| Ceramidase | | Pseudomonas | Okino, N. et al., |
| | | aeruginosa strain AN17/ | 1999. |
| | | Escherichia coli | |
| | | intracellular expression | |
| Ceramidase | calcium may alter activity | Pseudomonas/ | Wu, B. X. et al., |
| | | Escherichia coli | 2006. |
| Ceramidase | | Homo sapiens/Homo | Ferlinz, K. et al., |
| | | sapiens fibroblasts, | 2001. |
| | | glycosylation mutants | |
| | | activity not effected | |
| Cutinase | stable at 50° C., pH 7.0-9.2 | Fusarium solani pisi/ | Baptista, R. P. et al., |
| | | Escherichia coli WK-6, | 2003. |
| | | adsorption onto 100 nm | |
| | | diameter poly(methyl | |
| | | methacrylate) (PMMA) | |
| | | latex particles' surface | |
| Cutinase | | Fusarium solani pisi/ | Calado, C. R. et al., |
| | | Saccharomyces | 2004. |
| | | cerevisiae SU50 | |
| | | cultivation via batch or | |
| | | fed-batch cultures | |
| Cutinase | | Fusarium solani pisi/ | Calado, C. R. et al., |
| | | Saccharomyces | 2003.; Calado CR, et |
| | | cerevisiae SU50 fed- | al., 2002. |
| | | batch cultivation for | |
| | | secreted enzyme | |
| | | production | |
| Cutinase | | Fusarium solani pisi/ | Kepka, C. et al., |
| | | Escherichia coli | 2005. |
| | | intracellular expression | |
| | | as a typtophan-proline | |
| | | peptide tag fusion | |
| | | protein | |
| Cutinase | | Monilinia fructicola/ | Wang et al., 2002. |
| | | Pichia pastoris | |
| | | expression as a His- | |
| | | tagged fusion protein | |

Chemical modification of lipases, particularly the surface of such enzymes, has been used to improve organic solvent solubility, enhance activity, modify enantioselectivity, or a combination thereof. Such functional equivalents may be produced by reactions with stearic acid, polyethylene glycol (e.g., bonds to the free amino groups), pyridoxy]phosphate, tetranitromethane (sometimes followed by Na₂S₂O₄), glutaraldehyde (e.g., crosslinking to produce a crosslinked enzyme crystal know as a “CLEC”), polystyrene, polyacrylate, (R)-1-phenylethanol in combination with coating the enzyme's surface with a lipid at the molecular level; coating the enzyme's surface with a lipid or surfactant at the molecular level (e.g., didodecyl N-D-glucono-L-glutamate), forming a non-covalent complex formation with a surfactant (e.g., an ionic surfactant, a non-ionic surfactant), or any combination thereof as would be known to one of skill in the art [see, for example, “Methods in non-aqueous enzymology” (Gupta, M. N., Ed.) p. 85-89, 95 2000; Kurt Faber, “Biotransformations in Organic Chemistry, a Textbook, Third Edition.” pp. 357-376, 1997] For example, coupling a Pseudomonas sp., lipase with polyethylene glycol improved enzyme solubility in chlorinated hydrocarbons, benzene, and toluene (Okahata, Y. et al., 1995). In another example, coating a Rhizopus sp. lipase with didodecyl N-D-glucono-L-glutamate enhanced activity 100-fold and improved organic solubility, presumably because the surfactant acted as an interface to alter the lid conformation. (Okahata, Y. and Ijiro, K., 1992; Okahata, Y, Ijiro, K., 1988). Production of a Psuedomonas cepacia and Candida rugosa lipase CLECs enhanced stability, and the C. rugosa CLEC has enhanced enantioselectivity for ketoprofen (Lalonde, J. J. et al., 1995; Persichetti, R. A., 1996). The presence of some chemicals can also enhance stability, such as hexanol, which has been described as improving cutinase's stability (In “Engineering of/with Lipases” (F. Xavier Malcata., Ed.) p. 308, 1996). Chemical modification, such as for example, alkylation of lysine amino moieties with pyridoxal phosphate, nitration with tetranitromethane, with or without sodium hydrosulfite, improved enantiomeric selectivity of Candida rugosa lipase (Kurt Faber, “Biotransformations in Organic Chemistry, a Textbook, Third Edition.” Springer-verlag Berlin Heidelberg, pp. 114-115, 1997).

Other modifications that may be used are described herein, particularly in the processing of a biomolecular composition from a cell or biological material into a form for incorporation in a coating or surface treatment. All such techniques and compositions as are known to those of skill in the art and as described herein may be used in preparing a biomolecular composition, particularly in preparation of those compositions that comprise lipolytic enzyme (e.g., a cell-based particulate material comprising a lipolytic enzyme, a purified lipolytic enzyme).

b. OPH Functional Equivalents

Recombinant wild-type and mutant forms of the opd gene have been expressed, predominantly in Escherichia coli, for further characterization and analysis. Unless otherwise noted, the various OPH enzymes, whether wild-type or mutants, that act as functional equivalents were prepared using the OPH genes and encoded enzymes first isolated from Pseudomonas diminuta and Flavobacterium spp.

OPH normally binds two atoms of Zn²⁺ per monomer when endogenously expressed. While binding Zn²⁺, this enzyme is a stable dimeric enzyme, with a thermal temperature of melting (“Tₘ”) of approximately 75° C. and a conformational stability of approximately 40 killocalorie per mole (“kcal/mol”) (Grimsley, J. K. et al., 1997). However, structural analogs have been made wherein Co²⁺, Fe²⁺, Cu²⁺, Mn²⁺, Cd²⁺, or Ni²⁺ are bound instead to produce enzymes with altered stability and rates of activity (Omburo, G. A. et al., 1992). For example, Co²⁺ substituted OPH does possess a reduced conformational stability (˜22 kcal/mol). But this reduction in thermal stability is offset by the superior catalytic activity of Co²⁺ substituted OPH in degrading various OP compounds. For example, five-fold or greater rates of detoxification of sarin, soman, and VX were measured for Co²⁺ substituted OPH relative to OPH binding Zn²⁺ (Kolakoski, J. E. et al., 1997). It is contemplated that structural analogs of an OPH sequence may be prepared comprising a Zn²⁺, Co²⁺, Fe²⁺, Cu²⁺, Mn²⁺, Cd²⁺, Ni²⁺, or a combination thereof. Generally, changes in the bound metal can be achieved by using cell growth media during cell expression of the enzyme wherein the concentration of a metal present is defined, and/or removing the bound metal with a chelator (e.g., 1,10-phenanthroline; 8-hydroxyquinoline-5-sulfphonic acid; ethylenediaminetetraacetic acid) to produce an apo-enzyme, followed by reconstitution of a catalytically active enzyme by contact with a selected metal (Omburo, G. A. et al., 1992; Watkins, L. M. et al., 1997a; Watkins, L. M. et al., 1997b). It is further contemplated that structural analogs of an OPH sequence may be prepared to comprise one metal atom per monomer.

In an additional example, OPH structure analysis has been conducted using NMR (Omburo, G. A. et al., 1993). In a further example, the X-ray crystal structure for OPH has been determined (Benning, M. M. et al., 1994; Benning, M. M. et al., 1995; Vanhooke, J. L. et al., 1996), including the structure of the enzyme while binding a substrate, further identifying residues involved in substrate binding and catalytic activity (Benning, M. M. et al., 2000). From these structure evaluations, the amino acids His55, His57, His201, His 230, Asp301, and the carbamylated lysine, Lys169, have been identified as coordinating the binding of the active site metal. Additionally, the positively charged amino acids His55, His57, His201, His230, His254, and His257 are counter-balanced by the negatively charged amino acids Asp232, Asp233, Asp235, Asp 253, Asp301, and the carbamylated lysine Lys169 at the active site area. A water molecule and amino acids His55, His57, Lys169, His201, His230, and Asp301 are thought to be involved in direct metal binding. The amino acid Asp301 is thought to aid a nucleophilic attack by a bound hydroxide upon the phosphorus to promote cleavage of an OP compound, while the amino acid His354 may aid the transfer of a proton from the active site to the surrounding liquid in the latter stages of the reaction (Raushel, F. M., 2002). The amino acids His254 and His257 are not thought to be direct metal binding amino acids, but may be residues that interact (e.g., a hydrogen bond, a Van der Waal interaction) with each other and other active site residues, such as residues that directly contact a substrate or bind a metal atom. In particular, amino acid His254 is thought to interact with the amino acids His230, Asp232, Asp233, and Asp301. Amino acid His257 is thought to be a participant in a hydrophobic substrate-binding pocket. The active site pocket comprises various hydrophobic amino acids, Trp131, Phe132, Leu271, Phe306, and Tyr309. These amino acids may aid the binding of hydrophobic OP compounds (Benning, M. M. et al., 1994; Benning, M. M. et al., 1995; Vanhooke, J. L. et al., 1996). Electrostatic interactions may occur between phosphoryl oxygen, when present, and the side chains of Trp 131 and His201. Additionally, the side chains of amino acids Trp131, Phe132, and Phe306 are thought to be orientated toward the atom of the cleaved substrate's leaving group that was previously bonded to the phosphorus atom (Watkins, L. M. et al., 1997a).

Substrate binding subsites known as the small subsite, the large subsite, and the leaving group subsite have been identified (Benning, M. M. et al., 2000; Benning, M. M. et al., 1994; Benning, M. M. et al., 1995; Vanhooke, J. L. et al., 1996). The amino acids Gly6O, Ile106, Leu303, and Ser308 are thought to comprise the small subsite. The amino acids Cys59 and Ser61 are near the small subsite, but with the side chains thought to be orientated away from the subsite. The amino acids His254, His257, Leu271, and Met317 are thought to comprise the large subsite. The amino acids Trp131, Phe132, Phe306, and Tyr309 are thought to comprise the leaving group subsite, though Leu271 is sometimes considered part of this subsite as well (Watkins, L. M. et al., 1997a). Comparison of this opd product with the encoded sequence of the opdA gene from Agrobacterium radiobacter P230 revealed that the large subsite possessed generally larger residues that affected activity, specifically the amino acids Arg254, Tyr257, and Phe271 (Home, I. et al., 2002). Few electrostatic interactions are apparent from the X-ray crystal structure of the inhibitor bound by OPH, and it is thought that hydrophobic interactions and the size of the subsites affect substrate specificity, including steriospecificity for a stereoisomer, such as a specific enantiomer of an OP compound's chiral chemical moiety (Chen-Goodspeed, M. et al., 2001b).

Using the sequence and structural knowledge of OPH, numerous mutants of OPH comprising a sequence analog have been specifically produced to alter one or more properties relative to a substrate's cleavage rate (k_{cat}) and/or specificity (k_{cat}/Kₘ). Examples of OPH sequence analog mutants include H55C, H57C, C59A, G60A, S61A, I106A, I106G, W131A, W131F, W131K, F132A, F132H, F132Y, L136Y, L140Y, H201C, H230C, H254A, H254R, H254S, H257A, H257L, H257Y, L271A, L271Y, L303A, F306A, F306E, F306H, F306K, F306Y, S308A, S308G, Y309A, M317A, M317H, M317K, M317R, H55C/H57C, H55C/H201C, H55C/H230C, H57C/H201C, H57C/H230C, A80V/S365P, I106A/F132A, I106A/S308A, I106G/F132G, I106G/S308G, F132Y/F306H, F132H/F306H, F132H/F306Y, F132Y/F306Y, F132A/S308A, F132G/S308G, L182S/V310A, H201C/H230C, H254R/H257L, H55C/H57C/H201C, H55C/H57C/H230C, H55C/H201C/H230C, I106A/F132A/H257Y, I106A/F132A/H257W, I106G/F132G/S308G, L130M/H257Y/1274N, H257Y/1274N/S365P, H55C/H57C/H201C/H230C, I106G/F132G/H257Y/S308G or A14T/A80V/L185R/H257Y/1274N (Li, W.-S. et al., 2001; Gopal, S. et al., 2000; Chen-Goodspeed, M. et al., 2001a; Chen-Goodspeed, M. et al., 2001b; Watkins, L. M. et al., 1997a; Watkins, L. M. et al., 1997b; diSioudi, B. et al., 1999; Cho, C. M.-H. et al., 2002; Shim, H. et al., 1996; Raushel, F. M., 2002; Wu, F. et al., 2000a; diSioudi, B. D. et al., 1999).

For example, the sequence and structural information has been used in production of mutants of OPH possessing cysteine substitutions at the metal binding histidines His55, His57, His 201, and His230. OPH mutants H55C, H57C, H201C, H230C, H55C/H57C, H55C/H201C, H55C/H230C, H57C/H201C, H57C/H230C, H201C/H230C, H55C/H57C/H201C, H55C/H57C/H230C, H55C/H201C/H230C, H57C/H201C/H230C, and H55C/H57C/H201C/H230C were produced binding either Zn²⁺; Co²⁺ or Cd²⁺. The H57C mutant had between 50% (i.e., binding Cd²⁺, Zn²⁺) and 200% (i.e., binding Co²⁺) wild-type OPH activity for paraoxon cleavage. The H201C mutant had about 10% activity, the H230C mutant had less than 1% activity, and the H55C mutant bound one atom of Co²⁺ and possessed little detectable activity, but may still be useful if possessing a desirable property (e.g., enhanced stability) (Watkins, L. M., 1997b).

In an additional example, the sequence and structural information has been used in production of mutants of OPH possessing altered metal binding and/or bond-type cleavage properties. OPH mutants H254R, H257L, and H254R/H257L have been made to alter amino acids that are thought to interact with nearby metal-binding amino acids. These mutants also reduced the number of metal ions (i.e., Co²⁺, Zn²⁺) binding the enzyme dimer from four to two, while still retaining 5% to greater than 100% catalytic rates for the various substrates. These reduced metal mutants possess enhanced specificity for larger substrates such as NPPMP and demeton-S, and reduced specificity for the smaller substrate diisopropyl fluorophosphonate (diSioudi, B. et al., 1999). In a further example, the H254R mutant and the H257L mutant each demonstrated a greater than four-fold increase in catalytic activity and specificity against VX and its analog demeton S. The H257L mutant also demonstrated a five-fold enhanced specificity against soman and its analog NPPMP (diSioudi, B. D. et al., 1999).

In an example, specific mutants of OPH (a phosphotriesterase), were designed and produced to aid phosphodiester substrates to bind and be cleaved by OPH. These substrates either comprised a negative charge and/or a large amide moiety. A M317A mutant was created to enlarge the size of the large subsite, and M317H, M317K, and M317R mutants were created to incorporate a cationic group in the active site. The M317A mutant demonstrated a 200-fold cleavage rate enhancement in the presence of alkylamines, which were added to reduce the substrate's negative charge. The M317H, M317K, and M317R mutants demonstrated modest improvements in rate and/or specificity, including a 7-fold k_{cat}/Kₘ improvement for the M317K mutant (Shim, H. et al., 1998).

In a further example, the W131K, F132Y, F132H, F306Y, F306H, F306K, F306E, F132H/F306H, F132Y/F306Y, F132Y/F306H, and F132H/F306Y mutants were made to add or change the side chain of active site residues to form a hydrogen bond and/or donate a hydrogen to a cleaved substrate's leaving group, to enhance the rate of cleavage for certain substrates, such as phosphofluoridates. The F132Y, F132H, F306Y, F306H, F132H/F306H, F132Y/F306Y, F132Y/F306H, and F132H/F306Y mutants all demonstrated enhanced enzymatic cleavage rates, of about three- to ten-fold improvement, against the phosphonofluoridate, diisopropyl fluorophosphonate (Watkins, L. M. et al., 1997a).

In an additional example, OPH mutants W131F, F132Y, L136Y, L140Y, L271Y and H257L were designed to modify the active site size and placement of amino acid side chains to refine the structure of binding subsites to specifically fit the binding of a VX substrate. The refinement of the active site structure produced a 33% increase in cleavage activity against VX in the L136Y mutant (Gopal, S. et al., 2000).

Various mutants of OPH have been made to alter the steriospecificity, and in some cases, the rate of reaction, by substitutions in substrate binding subsites. For example, the C59A, G60A, S61A, I106A, W131A, F132A, H254A, H257A, L271A, L303A, F306A, S308A, Y309A, and M317A mutants of OPH have been produced to alter the size of various amino acids associated with the small subsite, the large subsite and the leaving group subsite, to alter enzyme activity and selectivity, including sterioselectivity, for various OP compounds. The G60A mutant reduced the size of the small subsite, and decreased both rate (k_{cat}) and specificity (k_{cat}/Kₐ) for Rₚ-enantiomers, thereby enhancing the overall specificity for some Sp-enantiomers to over 11,000:1. Mutants I106A and S308A, which enlarged the size of the small subsite, as well as mutant F132A, which enlarged the leaving group subsite, all increased the reaction rates for Rₚ-enantiomers and reduced the specificity for Sₚ-enantiomers (Chen-Goodspeed, M. et al., 2001a).

Additional mutants I106A/F132A, I106A/S308A, F132A/S308A, I106G, F132G, S308G, I106G/F132G, I106G/S308G, F132G/S308G, and I106G/F132G/S308G were produced to further enlarge the small subsite and leaving group subsite. These OPH mutants demonstrated enhanced selectivity for Rₚ-enantiomers. Mutants H254Y, H254F, H257Y, H257F, H257W, H257L, L271Y, L271F, L271W, M317Y, M317F, and M317W were produced to shrink the large subsite, with the H257Y mutant, for example, demonstrating a reduced selectivity for Sp-enantiomers (Chen-Goodspeed, M. et al., 2001b). Further mutants I106A/H257Y, F132A/H257Y, I106A/F132A/H257Y, I106A/H257Y/S308A, I106A/F132A/H257W, F132A/H257Y/S308A, I106G/H257Y, F132G/H257Y, I106G/F132G/H257Y, I106G/H257Y/S308G, and I106G/F132G/H257Y/S308G were made to simultaneously enlarge the small subsite and shrink the large subsite. Mutants such as H257Y, I106A/H257Y, I106G, I106A/F132A, and I106G/F132G/S308G were effective in altering steriospecificity for Sₚ:Rₚ enantiomer ratios of some substrates to less than 3:1 ratios. Mutants including F132A/H257Y, I106A/F132A/H257W, I106G/F132G/H257Y, and I106G/F132G/H257Y/S308G demonstrated a reversal of selectivity for Sₚ:Rₚ enantiomer ratios of some substrates to ratios from 3.6:1 to 460:1. In some cases, such a change in steriospecificity was produced by enhancing the rate of catalysis of a Rₚ enantiomer with little change on the rate of Sₚ enantiomer cleavage (Chen-Goodspeed, M. et al., 2001b; Wu, F. et al., 2000a).

Such alterations in sterioselectivity can enhance OPH performance against a specific OP compound that is a target of detoxification, including a CWA. Enlargement of the small subsite by mutations that substitute the IIle106 and Phe132 residues with the less bulky amino acid alanine and/or reduction of the large subsite by a mutation that substitutes His257 with the bulkier amino acid phenylalanine increased catalytic rates for the Sₚ-isomer; anddecreased the catalytic rates for the Rₚ-isomers of a sarin analog, thus resulting in a triple mutant, I106A/F132A4H257Y, with a reversed sterioselectivity such as a Sₚ:Rₚ preference of 30:1 for the isomers of the sarin analog. A mutant of OPH designated G60A has also been created with enhanced steriospecificity relative to specific analogs of enantiomers of sarin and soman (Li, W.-S. et al., 2001; Raushel, F. M., 2002). Of greater interest, these mutant forms of OPH have been directly assayed against sarin and soman nerve agents, and demonstrated enhanced detoxification rates for racemic mixtures of sarin or soman enantiomers. Wild-type OPH has a k_{cat} for sarin of 56 s⁻¹, while the I106A/F132A/H257Y mutant has k_{cat} for sarin of 1000 s⁻¹. Additionally, wild-type OPH has a k_{cat} for soman of 5 s⁻¹, while the G60A Mutant has k_{cat} for soman of 10 s⁻¹ (Kolakoski, Jan E. et al. 1997; Li, W.-S. et al., 2001).

It is also possible to produce a mutant enzyme with an enhanced enzymatic property against a specific substrate by evolutionary selection rather than rational design. Such techniques can screen hundreds or thousands of mutants for enhanced cleavage rates against a specific substrate. The mutants identified may possess substitutions at amino acids that have not been identified as directly comprising the active site, or its binding subsites, using techniques such as NMR, X-ray crystallography and computer structure analysis, but still contribute to activity for one or more substrates. For example, selection of OPH mutants based upon enhanced cleavage of methyl parathion identified the A80V/S365P, L182S/V310A, I274N, H257Y, H257Y/I274N/S365P, L130M/H257Y/I274N, and A14T/A80V/L185R/H257Y/I274N mutants as having enhanced activity. Amino acids Ile274 and Val310 are within 10 Å of the active site, though not originally identified as part of the active site from X-ray and computer structure analysis. However, mutants with substitutions at these amino acids demonstrated improved activity, with mutants comprising the I274N and H257Y substitutions particularly active against methyl parathion. Additionally, the mutant, A14T/A80V/L185R/H257Y/I274N, further comprising a L185R substitution, was active having a 25-fold improvement against methyl parathion (Cho, C. M.-H. et al., 2002).

In an example, a functional equivalent of OPH may be prepared that lacks the first 29-31 amino acids of the wild-type enzyme. The wild-type form of OPH endogenously or recombinantly expressed in Pseudomonas or Flavobacterium removes the first N-terminal 29 amino acids from the precursor protein to produce the mature, enzymatically active protein (Mulbry, W. and Karns, J., 1989; Serdar, C. M. et al., 1989). Recombinant expressed OPH in Gliocladium virens apparently removes part or all of this sequence (Dave, K. I. et al., 1994b). Recombinant expressed OPH in Streptomyces lividans primarily has the first 29 or 30 amino acids removed during processing, with a few percent of the functional equivalents having the first 31 amino acids removed (Rowland, S. S. et al., 1992). Recombinant expressed OPH in Spodoptera frugiperda cells has the first 30 amino acids removed during processing (Dave, K. I. et al., 1994a).

The 29 amino acid leader peptide sequence targets OPH enzyme to the cell membrane in Escherichia coli, and this sequence is partly or fully removed during cellular processing (Dave, K. I. et al., 1994a; Miller, C. E., 1992; Serdar, C. M. et al., 1989; Mulbry, W. and Karns, J., 1989). The association of OPH comprising the leader peptide sequence with the cell membrane in Escherichia coli expression systems seems to be relatively weak, as brief 15 second sonication releases most of the activity into the extracellular environment (Dave, K. I. et al., 1994a). For example, recombinant OPH often is expressed without this leader peptide sequence to enhance enzyme stability and expression efficiency in Escherichia coli (Serdar, C. M., et al. 1989). In another example, recombinant expression efficiency in Pseudomonas putida for OPH was improved by retaining this sequence, indicating that different species of bacteria may have varying preferences for a signal sequence (Walker, A. W. and Keasling, J. D., 2002). However, it is contemplated that the length of an enzymatic sequence may be readily modified to improve expression or other properties in a particular organism, or select a cell with a relatively good ability to express a biomolecule, in light of the present disclosures and methods in the art (see U.S. Pat. Nos. 6,469,145, 5,589,386 and 5,484,728)

In an example, recombinant OPH sequence-length mutants have been expressed wherein the first 33 amino acids of OPH have been removed, and a peptide sequence M-I-T-N-S added at the N-terminus (Omburo, G. A. et al., 1992; Mulbry, W. and Karns, J., 1989). Often removal of the 29 amino acid sequence is used when expressing mutants of OPH comprising one or more amino acid substitutions such as the C59A, G60A, S61A, I106A, W131A, F132A, H254A, H257A, L271A, L303A, F306A, S308A, Y309A, M317A, I106A/F132A, I106A/S308A, F132A/S308A, I106G, F132G, S308G, I106G/F132G, I106G/S308G, F132G/S308G, I106G/F132G/S308G, H254Y, H254F, H257Y, H257F, H257W, H257L, L271Y, L271W, M317Y, M317F, M317W, I106A/H257Y, F132A/H257Y, I106A/F132A/H257Y, I106A/H257Y/S308A, I106A/F132A/H257W, F132A/H257Y/S308A, I106G/H257Y, F132G/H257Y, I106G/F132G/H257Y, I106G/H257Y/S308G, and I106G/F132G/H257Y/S308G mutants (Chen-Goodspeed, M. et al., 2001a). In a further example, LacZ-OPH fusion protein mutants lacking the 29 amino acid leader peptide sequence and comprising an amino acid substitution mutant such as W131F, F132Y, L136Y, L140Y, H257L, L271L, L271Y, F306A, or F306Y have been recombinantly expressed (Gopal, S. et al., 2000).

In an additional example, OPH mutants that comprise additional amino acid sequences are also known in the art. An OPH fusion protein lacking the 29 amino acid leader sequence and possessing an additional C-terminal flag octapeptide sequence was expressed and localized in the cytoplasm of Escherichia coli (Wang, J. et al., 2001). In another example, nucleic acids encoding truncated versions of the ice nucleation protein (“InaV”) from Pseudomonas syringae have been used to construct vectors that express OPH-InaV fusion proteins in Escherichia coli. The InaV sequences targeted and anchored the OPH-InaV fusion proteins to the cells' outer membrane (Shimazu, M. et al., 2001a; Wang, A. A. et al., 2002). In a further example, a vector encoding a similar fusion protein was expressed in Moraxella sp., and demonstrated a 70-fold improved OPH activity on the cell surface compared to Escherichia coli expression (Shimazu, M. et al., 2001b). In a further example, fusion proteins comprising the signal sequence and first nine amino acids of lipoprotein, a transmembrane domain of outer membrane protein A (“Lpp-OmpA”), and either a wild-type OPH sequence or an OPH truncation mutant lacking the first 29 amino acids has been expressed in Escherichia coli. These OPH-Lpp-OmpA fusion proteins were targeted and anchored to the Escherichia coli cell membrane, though the OPH truncation mutant had 5% to 10% the activity of the wild-type OPH sequence (Richins, R. D. et al., 1997; Kaneva, I. et al., 1998). In one example, a fusion protein comprising N-terminus to C-terminus, a (His)6 polyhistidine tag, a green fluorescent protein (“GFP”), an enterokinase recognition site, and an OPH sequence lacking the 29 amino acid leader sequence has been expressed within Escherichia coli cells (Wu, C.-F. et al., 2000b, Wu, C.-F. et al., 2002). A similar fusion protein a (His)6 polyhistidine tag, an enterokinase recognition site, and an OPH sequence lacking the 29 amino acid leader sequence has also been expressed within Escherichia coli cells (Wu, C.-F. et al., 2002). Additionally, variations of these GFP-OPH fusion proteins have been expressed within Escherichia coli cells where an second enterokinase recognition site was placed at the C-terminus of the OPH gene fragment sequence, followed by a second OPH gene fragment sequence (Wu, C.-F. et al., 2001b). The GFP sequence produced fluorescence that was proportional to both the quantity of the fusion protein, and the activity of the OPH sequence, providing a fluorescent assay of enzyme activity and stability in GFP-OPH fusion proteins (Wu, C.-F. et al., 2000b, Wu, C.-F. et al., 2002).

In a further example, a fusion protein comprising an elastin-like polypeptide (“ELP”) sequence, a polyglycine linker sequence, and an OPH sequence was expressed in Escherichia coli (Shimazu, M. et al., 2002). In an additional example, a cellulose-binding domain at the N-terminus of an OPH fusion protein lacking the 29 amino acid leader sequence, and a similar fusion protein wherein OPH possessed the leader sequence, where both predominantly excreted into the external medium as soluble proteins by recombinant expression in Escherichia coli (Richins, R. D. et al., 2000).

c. Paraoxonase Functional Equivalents

Various chemical modifications to the amino acid residues of the recombinantly expressed human paraoxonase have been used to identify specific residues including tryptophans, histidines, aspartic acids, and glutamic acids as functioning in enzymatic activity for the cleavage of phenylacetate, paraoxon, chlorpyrifosoxon. and diazoxon. Additionally, comparison to conserved residues in human, mouse, rabbit, rat dog, chicken, and turkey paraoxonase enzymes was used to further identify amino acids for the production of specific mutants. Site-directed mutagenesis was used to alter the enzymatic activity of human paraoxonase through conservative and non-conservative substitutions, and thus clarify the specific amino acids functioning in enzymatic activity. Specific paraoxonase mutants include the sequence analogs E32A, E48A, E52A, D53A, D88A, D107A, H114N, D121A, H133N, H154N, H160N, W193A, W193F, W201A, W201F, H242N, H245N, H250N, W253A, W253F, D273A, W280A, W280F, H284N, or H347N.

The various paraoxonase mutants generally had different enzymatic properties. For example, W253A had a 2-fold greater k_{cat}; and W201F, W253A and W253F each had a 2 to 4 fold increase in k_{cat}, though W201F also had a lower substrate affinity. A non-conservative substitution mutant W280A had 1% wild-type paraoxonase activity, but the conservative substitution mutant W280F had similar activity as the wild-type paraoxonase (Josse, D. et al., 1999; Josse, D. et al., 2001).

d. Squid-Type DFPase Functional Equivalents

Various chemical modifications to the amino acid residues of the recombinantly expressed squid-type DFPase from Loligo vulgaris has been used to identify which specific types of residues of modified arginines, aspartates, cysteines, glutamates, histidines, lysines, and tyrosines, function in enzymatic activity for the cleavage of DFP. Modification of histidines generally reduced enzyme activity, and site-directed mutagenesis was used to clarify which specific histidines function in enzymatic activity. Specific squid-type DFPase mutants include the sequence analogs H181N, H224N, H274N, H219N, H248N, or H287N.

The H287N mutant lost about 96% activity, and is thought to act as a hydrogen acceptor in active site reactions. The H181N and H274N mutants lost between 15% and 19% activity, and are thought to help stabilize the enzyme. The H224N mutant gained about 14% activity, indicating that alterations to this residue may also affect activity (Hartleib, J. and Ruterjans, H., 2001b).

In a further example of squid-type DFPase functional equivalents, recombinant squid-type DFPase sequence-length mutants have been expressed wherein a (His)6 tag sequence and a thrombin cleavage site has been added to the squid-type DFPase (Hartleib, J. and Ruteijans, H., 2001a). In an additional example, a polypeptide comprising amino acids 1-148 of squid-type DFPase has been admixed with a polypeptide comprising amino acids 149-314 of squid-type DFPase to produce an active enzyme (Hartleib, J. and Rutejrans, H., 2001a).

8. Combinations of Biomolecules

It is contemplated that in various embodiments, a composition may comprise one or more selected biomolecules, with an enzyme being a type of biomolecule in certain facets. It is contemplated that in specific embodiments, a compositionmay comprise an endogenously expressed wild-type enzyme, a recombinant enzyme, or a combination thereof. In specific aspects, a recombinant enzyme comprises a wild-type enzyme, a functional equivalent enzyme, or a combination thereof. Numerous examples of enzymes with different properties are described herein, and any such enzyme in the art is contemplated for inclusion in a composition.

It is contemplated that a combination of biomolecules may be selected for inclusion in the biomolecular composition, coating and/or paint, to improve one or more properties of such a composition. Thus, a composition may comprise 1 to 1000 or more different selected biomolecules of interest, including all intermediate ranges and combinations thereof. For example, as various enzymes have differing binding properties, catalytic properties, stability properties, properties related to environmental safety, etc, one may select a combination of enzymes to confer a more desirable range of properties to a composition. In a specific example, it is contemplated that lipolytic enzymes, with differing but desirable abilities to cleave the lipid substrates, may be admixed to confer a more desirable range of catalytic properties to a composition than would be achieved by the selection of a single lipolytic enzyme. In a specific example, a coating may comprise a plurality of biomolecular compositions. In an additional specific example, one or more layers of a multicoat system comprise one or more different biomolecular compositions to confer differing properties between one layer and at least a second layer of the multicoat system.

### C. Recombinantly Produced Enzymes

In certain aspects, an enzymemay be biologically produced in cell, tissue and/or organism transformed with a genetic expression vector. As used herein, an “expression vector” refers to a carrier nucleic acid molecule, into which a nucleic acid sequence can be inserted, wherein the nucleic acid sequence is capable of being transcribed into a ribonucleic acid (“RNA”) molecule after introduction into a cell. Usually an expression vector comprises deoxyribonucleic acid (“DNA”). As used herein, an “expression system” refers to an expression vector, and may further comprise additional reagents needed to promote insertion of a nucleic acid sequence, introduction into a cell, transcription and/or translation. As used herein, a “vector,” refers to a carrier nucleic acid molecule into which a nucleic acid sequence can be inserted for introduction into a cell. Certain vectors are capable of replication of the vector and/or any inserted nucleic acid sequence in a cell. For example, a viral vector may be used in conjunction with either an eukaryotic or prokaryotic host cell, particularly one that is permissive for replication or expression of the vector. A cell that is capable of being transformed with a vector is known herein as a “host cell.”

In general embodiments, the inserted nucleic acid sequence encodes for at least part of a gene product. In some embodiments wherein the nucleic acid sequence is transcribed into an RNA molecule, the RNA molecule is then translated into a proteinaceous molecule. As used herein, a “gene” refers to a nucleic acid sequence isolated from an organism, and/or man-made copies or mutants thereof, that comprises a nucleic acid sequence capable of being transcribed and/or translated in an organism. A “gene product” is the transcribed RNA and/or translated proteinaceous molecule from a gene. Often, partial nucleic acid sequences of a gene, known herein as a “gene fragment,” are used to produce a part of the gene product. Many gene and gene fragment sequences are known in the art, and are both commercially available and/or publicly disclosed at a database such as Genbank. It is contemplated that a gene and/or a gene fragment can be used to recombinantly produce an enzyme. It is further contemplated that a gene and/or a gene fragment can be use in construction of a fusion protein comprising an enzyme.

In certain embodiments, a nucleic acid sequence such as a nucleic acid sequence encoding an enzyme, or any other desired RNA or proteinaceous molecule (as well as a nucleic acid sequence comprising a promoter, a ribosome binding site, an enhancer, a transcription terminator, an origin of replication, or other nucleic acid sequences, including but not limited to those described herein may be recombinantly produced or synthesized using any method or technique in the art in various combinations. [In “Molecular Cloning” (Sambrook, J., and Russell, D. W., Eds.) 3rd Edition, Cold Spring Harbor, N.Y.: Cold Spring Harbor Laboratory Press, 2001; In “Current Protocols in Molecular Biology” (Chanda, V. B. Ed.) John Wiley & Sons, 2002; In “Current Protocols in Cell Biology” (Morgan, K. Ed.) John Wiley & Sons, 2002; In “Current Protocols in Nucleic Acid Chemistry” (Harkins, E. W. Ed.) John Wiley & Sons, 2002; In “Current Protocols in Protein Science” (Taylor, G. Ed.) John Wiley & Sons, 2002; In “Current Protocols in Pharmacology” (Taylor, G. Ed.) John Wiley & Sons, 2002; In “Current Protocols in Cytometry” (Robinson, J. P. Ed.) John Wiley & Sons, 2002; In “Current Protocols in Immunology” (Coico, R. Ed.) John Wiley & Sons, 2002]. For example, a gene and/or a gene fragment encoding the enzyme of interest may be isolated and/or amplified through polymerase chain reaction (“PCR™”) technology. Often such nucleic acid sequence is readily available from a public database and/or a commercial vendor, as previously described.

Nucleic acid sequences, called codons, encoding for each amino acid are used to copy and/or mutate a nucleic acid sequence to produce a desired mutant in an expressed amino acid sequence. Codons comprise nucleotides such as adenine (“A”), cytosine (“C”), guanine (“G”), thymine (“T”) and uracil (“U”). The common amino acids are generally encoded by the following codons: alanine is encoded by GCU, GCC, GCA, or GCG; arginine is encoded by CGU, CGC, CGA, CGG, AGA, or AGG; aspartic acid is encoded by GAU or GAC; asparagine is encoded by AAU or AAC; cysteine is encoded by UGU or UGC; glutamic acid is encoded by GAA or GAG; glutamine is encoded by CAA or CAG; glycine is encoded by GGU, GGC, GGA, or GGG; histidine is encoded by CAU or CAC; isoleucine is encoded by AUU, AUC, or AUA; leucine is encoded by UUA, UUG, CUU, CUC, CUA, or CUG; lysine is encoded by AAA or AAG; methionine is encoded by AUG; phenylalanine is encoded by UUU or UUC; proline is encoded by CCU, CCC, CCA, or CCG; serine is encoded by AGU, AGC, UCU, UCC, UCA, or UCG; threonine is encoded by ACU, ACC, ACA, or ACG; tryptophan is encoded by UGG; tyrosine is encoded by UAU or UAC; and valine is encoded by GUU, GUC, GUA, or GUG.

A mutation in a nucleic acid encoding a proteinaceous molecule may be introduced into the nucleic acid sequence through any technique in the art. Sucha mutation may be bioengineered to a specific region of a nucleic acid comprising one or more codons using a technique such as site-directed mutagenesis or cassette mutagenesis. Numerous examples of phosphoric triester hydrolase mutants have been produced using site-directed mutagenesis or cassette mutagenesis, and are described herein.

It is contemplated that for recombinant expression, the choice of codons may be made to mimic the host cell's molecular biological activity, to improve the efficiency of expression from an expression vector. For example, codons may be selected to match the preferred codons used by a host cell in expressing endogenous proteins. In some aspects, the codons selected may be chosen to approximate the G-C content of an expressed gene and/or a gene fragment in a host cell's genome, or the G-C content of the genome itself. In other aspects, a host cell may be genetically altered to recognize more efficiently use a variety of codons, such as Escherichia coli host cells that are dnaY gene positive (Brinkmann, U. et al., 1989).

1. General Expression Vector Components and Use

An expression vector may comprise specific nucleic acid sequences such as a promoter, a ribosome binding site, an enhancer, a transcription terminator, an origin of replication, or other nucleic acid sequence, including but not limited to those described herein, in various combinations. A nucleic acid sequence can be “exogenous,” which means that it is foreign to the cell into which the vector is being introduced or that the sequence is homologous to a sequence in the cell, but in a position within the host cell nucleic acid in which the sequence is ordinarily not found. An expression vector may have one or more nucleic acid sequences removed by restriction enzyme digestion, modified by mutagenesis, and/or replaced with another more appropriate nucleic acid sequence, for transcription and/or translation in a host cell suitable for the expression vector selected.

One of skill in the art can construct a vector through standard recombinant techniques in the art. Further, one of skill in the art would know how to express a vector to transcribe a nucleic acid sequence and/or translate its cognate proteinaceous molecule. One of skill in the art would further understand the conditions under which to incubate all of the above described host cells to maintain them and to permit replication of a vector. Also understood and known are techniques and conditions that would allow large-scale production of a vector, as well as production of a nucleic acid sequence encoded by a vector into an RNA molecule and/or translation of the RNA molecule into a cognate proteinaceous molecule.

In certain embodiments, a cell may express multiple gene and/or gene fragment products from the same vector, and/or express more than one vector. Often this occurs simply as part of the normal function of a multi-vector expression system. For example, one gene or gene fragment is often used to produce a repressor that suppresses the activity of a promoter that controls the expression of a gene or a gene fragment of interest. The repressor gene and the desired gene may be on different vectors. However, multiple gene, gene fragment and/or expression systems may be used to express an enzymatic sequence of interest and another gene or gene fragment that is desired for a particular function. In an example, recombinant Pseudomonas putida has co-expressed OPH from one vector, and the multigenes encoding the enzymes for converting p-nitrophenol to β-ketoadipate from a different vector. The expressed OPH catalyzed the cleavage of parathion to p-nitrophenol. The additionally expressed recombinant enzymes converted the p-nitrophenol, which is a moderately toxic compound, to β-ketoadipate, thereby detoxifying both an OP compound and the byproducts of its hydrolysis (Walker, A. W. and Keasling, J. D., 2002). In a further example, Escherichia coli cells expressed a cell surface targeted INPNC-OPH fusion protein from one vector to detoxify OP compounds, and co-expressed from a different vector a cell surface targeted Lpp-OmpA-cellulose binding domain fusion protein to immobilize the cell to a cellulose support (Wang, A. A. et al., 2002). In an additional example, a vector co-expressed an antisense RNA sequence to the transcribed stress response gene σ³² and OPH in Escherichia coli. The antisense σ³² RNA was used to reduce the cell's stress response, including proteolytic damage, to an expressed recombinant proteinaceous molecule. A six-fold enhanced specific activity of expressed OPH enzyme was seen (Srivastava, R. et al., 2000). In a further example, multiple OPH fusion proteins were expressed from the same vector using the same promoter but separate ribosome binding sites (Wu, C.-F. et al., 2001b).

An expression vector generally comprises a plurality of functional nucleic acid sequences that either comprise a nucleic acid sequence with a molecular biological function in a host cell, such as a promoter, an enhancer, a ribosome binding site, a transcription terminator, etc, and/or encode a proteinaceous sequence, such as a leader peptide, a polypeptide sequence with enzymatic activity, a peptide or polypeptide with a binding property, etc. A nucleic acid sequence may comprise a “control sequence,” which refers to a nucleic acid sequence that functions in the transcription and possibly translation of an operatively linked coding sequence in a particular host cell. As used herein, an “operatively linked” or “operatively positioned” nucleic acid sequence refers to the placement of one nucleic acid sequence into a functional relationship with another nucleic acid sequence. Vectors and expression vectors may further comprise one or more nucleic acid sequences that serve other functions as well and are described herein.

The various functional nucleic acid sequences that comprise an expression vector are operatively linked so to position the different nucleic acid sequences for function in a host cell. In certain cases, the functional nucleic acid sequences may be contiguous such as placement of a nucleic acid sequence encoding a leader peptide sequence in correct amino acid frame with a nucleic acid sequence encoding a polypeptide comprising a polypeptide sequence with enzymatic activity. In other cases, the functional nucleic acid sequences may be non-contiguous such as placing a nucleic acid sequence comprising an enhancer distal to a nucleic acid sequence comprising such sequences as a promoter, an encoded proteinaceous molecule, a transcription termination sequence, etc. One or more nucleic acid sequences may be operatively linked using methods in the art, particularly ligation at restriction sites that may pre-exist in a nucleic acid sequence or be added through mutagenesis.

A “promoter” is a control sequence that is a region of a nucleic acid sequence at which initiation and rate of transcription are controlled. In the context of a nucleic acid sequence comprising a promoter and an additional nucleic acid sequence, particularly one encoding a gene or gene fragment's product, the phrases “operatively linked,” “operatively positioned,” “under control,” and “under transcriptional control” mean that a promoter is in a correct functional location and/or orientation in relation to the additional nucleic acid sequence to control transcriptional initiation and/or expression of the additional nucleic acid sequence. A promoter may contain genetic elements at which regulatory proteins and molecules may bind such as RNA polymerase and other transcription factors. A promoter employed may be constitutive, tissue-specific, inducible, and/or useful under the appropriate conditions to direct high level expression of the introduced nucleic acid sequence, such as is in the large-scale production of a recombinant proteinaceous molecule. Examples of a promoter include a lac, a tac, an amp, a heat shock promoter of a P-element of Drosophila, a baculovirus polyhedron gene promoter, or a combination thereof. In a specific example, the nucleic acids encoding OPH have been expressed using the polyhedron promoter of a baculoviral expression vector (Dumas, D. P. et al., 1990). In a further example, a Cochliobolus heterostrophus promoter, prom1, has been used to express a nucleic acid encoding OPH (Dave, K. I. et al., 1994b).

The promoter may be endogenous or heterologous. An “endogenous promoter” comprises one naturally associated with a gene or sequence, as may be obtained by isolating the 5′ non-coding sequences located upstream of the coding segment and/or exon. Alternatively, the coding nucleic acid sequence may be positioned under the control of a “heterologous promoter” or “recombinant promoter,” which refers to a promoter that is not normally associated with a nucleic acid sequence in its natural environment.

A specific initiation signal also may be required for efficient translation of a coding sequence by the host cell. Such a signal may include an ATG initiation codon (“start codon”) and/or an adjacent sequence. Exogenous translational control signals, including the ATG initiation codon, may need to be provided. Techniues of the art would readily be capable of determining this and providing the signals. The initiation codon should be “in-frame” with the reading frame of the desired coding sequence to ensure translation of the entire insert. The exogenous translational control signal and/or an initiation codon can be either natural or synthetic. The efficiency of expression may be enhanced by the inclusion of an appropriate transcription enhancer.

A promoter may or may not be used in conjunction with an “enhancer,” which refers to a cis-acting regulatory sequence involved in the transcriptional activation of a nucleic acid sequence. An enhancer may be one naturally associated with a nucleic acid sequence, located either downstream or upstream of that sequence. A recombinant or heterologous enhancer refers also to an enhancer not normally associated with a nucleic acid sequence in its natural environment. Such a promoter and/or enhancer may include a promoter and/or enhancer of another gene, a promoter and/or enhancer isolated from any other prokaryotic, viral, or eukaryotic cell, a promoter and/or enhancer not “naturally occurring,” i.e., a promoter and/or enhancer comprising different elements of different transcriptional regulatory regions, and/or mutations that alter expression. In addition to producing a nucleic acid sequence comprising a promoter and/or enhancer synthetically, a sequence may be produced using recombinant cloning and/or nucleic acid amplification technology, including PCR™, in connection with the compositions disclosed herein (U.S. Pat. No. 4,683,202, U.S. Pat. No. 5,928,906).

It is contemplated to employ a promoter and/or enhancer that effectively directs the expression of the nucleic acid sequence in the cell type, chosen for expression. Those of skill in the art of molecular biology generally know the use of promoters, enhancers, and cell type combinations for expression. Furthermore, it is contemplated the control sequences that direct transcription and/or expression of sequences within non-nuclear organelles, including eukaryotic organelles such as mitochondria, chloroplasts, and the like, can be employed as well.

Vectors can include a multiple cloning site (“MCS”), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme which functions at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable an exogenous nucleic acid sequence to be ligated to the vector. “Ligation” refers to the process of forming phosphodiester bonds between two nucleic acid fragments, which may or may not be contiguous with each other. Techniques involving restriction enzymes and ligation reactions in the art of recombinant technology may be applied.

A “fusion protein,” as used herein, is an expressed contiguous amino acid sequence comprising a proteinaceous molecule of interest and one or more additional peptide or polypeptide sequences. The additional peptide or polypeptide sequence generally provides an useful additional property to the fusion protein, including but not limited to, targeting the fusion protein to a particular location within or external to the host cell (e.g., a signal peptide); promoting the ease of purification and/or detection of the fusion protein (e.g., a tag, a fusion partner); promoting the ease of removal of one or more additional sequences from the peptide or polypeptide of interest (e.g., a protease cleavage site); and separating one or more sequences of the fusion protein to allow improved activity or function of the sequence(s) (e.g., a linker sequence).

As used herein a “tag” is a peptide sequence operatively associated to the sequence of another peptide or polypeptide sequence. Examples of a tag include a His-tag, a strep-tag, a flag-tag, a T7-tag, a S-tag, a HSV-tag, a polyarginine-tag, a polycysteine-tag, a polyaspartic acid-tag, a polyphenylalanine-tag, or a combination thereof. A His-tag is 6 or 10 amino acids in length, and can be incorporated at the N-terminus, C-terminus or within an amino acid sequence for use in detection and purification. A His tag binds affinity columns comprising nickel, and is eluted using low pH conditions or with imidazole as a competitor (Unger, T. F., 1997). A strep-tag is 10 amino acids in length, and can be incorporated at the C-terminus. A strep-tag binds streptavidin or affinity resins that comprise streptavidin. A flag-tag is 8 amino acids in length, and can be incorporated at the N-terminus or C-terminus of an amino acid sequence for use in purification. A T7-tag is 11 or 16 amino acids in length, and can be incorporated at the N-terminus or within an amino acid sequence for use in purification. A S-tag is 15 amino acids in length, and can be incorporated at the N-terminus, C-terminus or within an amino acid sequence for use in detection and purification. A HSV-tag is 11 amino acids in length, and can be incorporated at the C-terminus of an amino acid sequence for use in purification. The HSV tag binds an anti-HSV antibody in purification procedures (Unger, T. F., 1997). A polyarginine-tag is 5 to 15 amino acids in length, and can be incorporated at the C-terminus of an amino acid sequence for use in purification. A polycysteine-tag, is 4 amino acids in length, and can be incorporated at the N-terminus of an amino acid sequence for use in purification. A polyaspartic acid-tag can be 5 to 16 amino acids in length, and can be incorporated at the C-terminus of an amino acid sequence for use in purification. A polyphenylalanine-tag is 11 amino acids in length, and can be incorporated at the N-terminus of an amino acid sequence for use in purification.

In one example, a (His)6 tag sequence has been used to purify fusion proteins comprising GFP-OPH or OPH using immobilized metal affinity chromatography (“IMAC”) (Wu, C.-F. et al., 2000b; Wu, C.-F. et al., 2002). In a further example, a (His)6 tag sequence followed by a thrombin cleavage site has been used to purify fusion proteins comprising squid-type DFPase using IMAC (Hartleib, J. and Ruterjans, H., 2001a). In a further example, an OPH fusion protein comprising a C-terminal flag has been expressed (Wang, J. et al., 2001).

As used herein a “fusion partner” is a polypeptide that is operatively associated to the sequence of another peptide or polypeptide of interest. Properties that a fusion partner can confer to a fusion protein include, but are not limited to, enhanced expression, enhanced solubility, ease of detection, and/or ease of purification of a fusion protein. Examples of a fusion partner include a thioredoxin, a cellulose-binding domain, a calmodulin binding domain, an avidin, a protein A, a protein G, a glutathione-S-transferase, a chitin-binding domain, an ELP, a maltose-binding domain, or a combination thereof. Thioredoxin can be incorporated at the N-terminus or C-terminus of an amino acid sequence for use in purification. A cellulose-binding domain binds a variety of resins comprising cellulose or chitin (Unger, T. F., 1997). A calmodulin-binding domain binds affinity resins comprising calmodulin in the presence of calcium, and allows elution of the fusion protein in the presence of ethylene glycol tetra acetic acid (“EGTA”) (Unger, T. F., 1997). Avidin is useful in purification or detection. A protein A or a protein G binds a variety of anti-bodies for ease of purification. Protein A is generally bound to an IgG sepharose resin (Unger, T. F., 1997). Streptavidin is useful in purification or detection. Glutathione-S-transferase can be incorporated at the N-terminus of an amino acid sequence for use in detection or purification. Glutathione-S-transferase binds affinity resins comprising glutathione (Unger, T. F., 1997). An elastin-like polypeptide comprises repeating sequences (e.g., 78 repeats) which reversibly converts itself, and thus the fusion protein, from an aqueous soluble polypeptide to an insoluble polypeptide above an empirically determined transition temperature. The transition temperature is affected by the number of repeats, and can be determined spectrographically using techniques known in the art, including measurements at 655 nano meters (“nm”) over a 4° C. to 80° C. range (Urry, D. W. 1992; Shimazu, M. et al., 2002). A chitin-binding domain comprises an intein cleavage site sequence, and can be incorporated at the C-terminus for purification. The chitin-binding domain binds affinity resins comprising chitin, and an intein cleavage site sequence allows the self-cleavage in the presence of thiols at reduced temperature to release the peptide or polypeptide sequence of interest (Unger, T. F., 1997). A maltose-binding domain can be incorporated at the N-terminus or C-terminus of an amino acid sequence for use in detection or purification. A maltose-binding domain sequence usually further comprises a ten amino acid poly asparagine sequence between the maltose binding domain and the sequence of interest to aid the maltose-binding domain in binding affinity resins comprising amylose (Unger, T. F., 1997).

In an example, a fusion protein comprising an elastin-like polypeptide sequence and an OPH sequence has been expressed (Shimazu, M. et al., 2002). In a further example, a cellulose-binding domain-OPH fusion protein has also been recombinantly expressed (Richins, R. D. et al., 2000). In an additional example, a maltose binding protein-E3 carboxylase fusion protein has been recombinantly expressed (Claudianos, C. et al., 1999)

A protease cleavage site promotes proteolytic removal of the fusion partner from the peptide or polypeptide of interest. Often, a fusion protein is bound to an affinity resin, and cleavage at the cleavage site promotes the ease of purification of a peptide or polypeptide of interest with most or all of the tag or fusion partner sequence removed (Unger, T. F., 1997). Examples of protease cleavage sites used in the art include the factor Xa cleavage site, which is four amino acids in length; the enterokinase cleavage site, which is five amino acids in length; the thrombin cleavage site, which is six amino acids in length; the rTEV protease cleavage site, which is seven amino acids in length; the 3C human rhino virus protease, which is eight amino acids in length; and the PreScission™ cleavage site, which is eight amino acids in length. In an example, an enterokinase recognition site was used to separate an OPH sequence from a fusion partner (Wu, C.-F. et al., 2000b; Wu, C.-F. et al., 2001b).

In an eukaryotic expression system (e.g., a fungal expression system), the “terminator region” or “terminator” may also comprise a specific DNA sequence that permits site-specific cleavage of the new transcript so as to expose a polyadenylation site. This signals a specialized endogenous polymerase to add a stretch of adenosine nucleotides (“polyA”) of about 200 in number to the 3′ end of the transcript. RNA molecules modified with this polyA tail appear to more stable and are translated more efficiently. Thus, in other embodiments involving an eukaryote, in some embodiments a terminator comprises a signal for the cleavage of the RNA, and in some aspects the terminator signal promote polyadenylation of the message. The terminator and/or polyadenylation site elements can serve to enhance message levels and/or to minimize read through from the cassette into other sequences.

A terminator contemplated includes any known terminator of transcription, including but not limited to those described herein. For example, a termination sequence of a gene, such as for example, a bovine growth hormone terminator or a viral termination sequence, such as for example a SV40 terminator. In certain embodiments, the termination signal may be a lack of transcribable or translatable sequence, such as due to a sequence truncation. In one example, a trpC terminator from Aspergillus nidulans has been used in the expression of recombinant OPH (Dave, K. I. et al., 1994b).

In expression, particularly eukaryotic expression, one will typically include a polyadenylation signal to effect proper polyadenylation of the transcript. Any such sequence may be employed. Some embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

To propagate a vector in a host cell, it may contain one or more origins of replication sites (“ori”), which is a specific nucleic acid sequence at which replication is initiated. Alternatively an autonomously replicating sequence (“ARS”) can be employed if the host cell is yeast.

Various types of prokaryotic and/or eukaryotic expression vectors are known in the art. Examples of types of expression vectors include a bacterial artificial chromosome (“BAC”), a cosmid, a plasmid [e.g., a pMB1/colE1 derived plasmid such as pBR322, pUC18; a Ti plasmid of Agrobacterium tumefaciens derived vector (Rogers, S. G. et al., 1987)], a virus (e.g., a bacteriophage such as a bacteriophage M13, an animal virus, a plant virus), or a yeast artificial chromosome (“YAC”). Some vectors, known herein as “shuttle vectors” may employ control sequences that allow it to be replicated and/or expressed in both prokaryotic and eukaryotic cells [e.g., a wheat dwarf virus (“WDV”) pW1-11 or pW1-GUS shuttle vector (Ugaki, M. et al., 1991)]. An expression vector operatively linked to a nucleic acid sequence encoding an enzymatic sequencemay be constructed using techniques known to those of skill in the art in light of the present disclosures [In “Molecular Cloning” (Sambrook, J., and Russell, D. W., Eds.) 3rd Edition, Cold Spring Harbor, N.Y.: Cold Spring Harbor Laboratory Press, 2001; In “Current Protocols in Molecular Biology” (Chanda, V. B. Ed.) John Wiley & Sons, 2002; In “Current Protocols in Nucleic Acid Chemistry” (Harkins, E. W. Ed.) John Wiley & Sons, 2002; In “Current Protocols in Protein Science” (Taylor, G. Ed.) John Wiley & Sons, 2002; In “Current Protocols in Cell Biology” (Morgan, K. Ed.) John Wiley & Sons, 2002].

Numerous expression systems exist that comprise at least a part or all of the compositions discussed above. Prokaryote- and/or eukaryote-based systems can be employed to produce nucleic acid sequences, or their cognate polypeptides, proteins and peptides. Many such systems are widely available, including those provide by commercial vendors, as would be known to those of skill in the art. For example, an insect cell/baculovirus system can produce a high level of protein expression of a heterologous nucleic acid sequence, such as described in U.S. Pat. Nos. 5,871,986, 4,879,236, both incorporated herein by reference, and which can be bought, for example, under the name MAXBAC® 2.0 from INVITROGEN® and BACPACK™ BACULOVIRUS EXPRESSION SYSTEM FROM CLONTECH®. In an additional example of an expression system include STRATAGENE®'S COMPLETE CONTROL™ Inducible Mammalian Expression System, which involves a synthetic ecdysone-inducible receptor, or its pET Expression System, an Escherichia coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. In a specific example, E3 carboxylase enzymatic sequences and phosphoric triester hydrolase functional equivalents have been recombinantly expressed in a BACPACK™ Baculovirus Expression System From CLONTECH® (Newcomb, R. D. et al., 1997; Campbell, P. M. et al., 1998). In certain embodiments, a biomolecule may be expressed in a plant cell (e.g., a corn cell), using techniques such as those described in U.S. Pat. Nos. 6,504,085, 6,136,320, 6,087,558, 6034,298, 5,914,123, and 5,804,694.

2. Prokaryotic Expression Vectors and Use

In some embodiments, a prokaryote such as a bacterium comprises a host cell. In specific aspects, the bacterium host cell comprises a Gram-negative bacterium cell. Various prokaryotic host cells have been used in the art with expression vectors, and it is contemplated that any prokaryotic host cell known in the art may be used to express a peptide or polypeptide comprising an enzyme sequence.

An expression vector for use in prokaryotic cells generally comprises nucleic acid sequences such as, a promoter, a ribosome binding site (e.g., a Shine-Delgarno sequence), a start codon, a multiple cloning site, a fusion partner, a protease cleavage site, a stop codon, a transcription terminator, an origin of replication, a repressor, and/or any other additional nucleic acid sequence that would be used in such an expression vector in the art [see, for example, Makrides, S. C., 1996; Hannig, G. and Makrides, S. C., 1998; Stevens, R. C., 2000; In “Molecular Cloning” (Sambrook, J., and Russell, D. W., Eds.) 3rd Edition, Cold Spring Harbor, N.Y.: Cold Spring Harbor Laboratory Press, 2001; In “Current Protocols in Molecular Biology” (Chanda, V. B. Ed.) John Wiley & Sons, 2002; In “Current Protocols in Nucleic Acid Chemistry” (Harkins, E. W. Ed.) John Wiley & Sons, 2002; In “Current Protocols in Protein Science” (Taylor, G. Ed.) John Wiley & Sons, 2002; In “Current Protocols in Cell Biology” (Morgan, K. Ed.) John Wiley & Sons, 2002].

A promoter generally is positioned 10 to 100 nucleotides 5′ to a nucleic acid sequence comprising a ribosome binding site. Examples of promoters that have been used in a prokaryotic cell includes a T5 promoter, a lac promoter, a tac promoter, a trc promoter, an araBAD promoter, a P_{L} promoter, a T7 promoter, a T7-lac operator promoter, and variations thereof. The T5 promoter is regulated by the lactose operator. A lac promoter (e.g., a lac promoter, a lacUV5 promoter), a tac promoter (e.g., a taci promoter, a tacII promoter), a T7-lac operator promoter or a trc promoter are each suppressed by a lacI repressor, a more effective lacI^{Q} repressor or an even stronger lacI^{Q1} repressor (Glascock, C. B. and Weickert, M. J., 1998). Isopropyl-β-D-thiogalactoside (“IPTG”) is used to induce lac, tac, T7-lac operator and trc promoters. An araBAD promoter is suppressed by an araC repressor, and is induced by 1-arabinose. A P_{L} promoter or a T7 promoter are each suppressed by a λcIts857 repressor, and induced by a temperature of 42° C. Nalidixic acid may be used to induce a P_{L} promoter.

In an example, recombinant amino acid substitution mutants of OPH have been expressed in Escherichia coli using a lac promoter induced by IPTG (Watkins, L. M. et al., 1997b). In another example, recombinant wild type and a signal sequence truncation mutant of OPH was expressed in Pseudomonas putida under control of a lactac and tac promoters (Walker, A. W. and Keasling, J. D., 2002). In a further example, an OPH-Lpp-OmpA fusion protein has been expressed in Escherichia coli strains JM105 and XL1-Blue using a constitutive lpp-lac promoter or a tac promoter induced by IPTG and controlled by a lacI^{Q} repressor (Richins, R. D. et al., 1997; Kaneva, I. et al., 1998; Mulchandani, A. et al., 1999b). In an additional example, a cellulose-binding domain-OPH fusion protein has also been recombinantly expressed under the control of a T7 promoter (Richins, R. D. et al., 2000). In a further example, recombinant Altermonas sp. JD6.5 OPAA has been expressed under the control of a tre promoter in Escherichia coli (Cheng, T.-C. et al., 1999). In an additional example, a (His)6 tag sequence-thrombin cleavage site-squid-type DFPase has been expressed using a Ptac promoter in Escherichia coli (Hartleib, J. and Ruterjans, H., 2001a).

A ribosome binding site functions in transcription initiation, and is usually positioned 4 to 14 nucleotides 5′ from the start codon. A start codon signals initiation of transcription. A multiple cloning site comprises restriction sites for incorporation of a nucleic acid sequence encoding a peptide or polypeptide of interest.

A stop codon signals translation termination. The vectors or constructswill generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be used in vivo to achieve desirable message levels. A transcription terminator signals the end or transcription and often enhances mRNA stability. Examples of a transcription terminator include a rrnB T1 or a rrnB T2 transcription terminator (Unger, T. F., 1997). An origin of replication regulates the number of expression vector copies maintained in a transformed host cell.

A selectable marker usually provides a transformed cell resistance to an antibiotic. Examples of a selectable marker used in a prokaryotic expression vector include a β-lactamase, which provides resistance to antibiotic such as an ampicillin or a carbenicillin; a tet gene product, which provides resistance to a tetracycline, or a Km gene product, which provides resistance to a kanamycin. A repressor regulatory gene suppresses transcription from the promoter. Examples of repressor regulatory genes include the lacI, lacI^{q}, or lacI^{Q1} repressors (Glascock, C. B. and Weickert, M. J., 1998). Often, the host cell's genome, or additional nucleic acid vector co-transfected into the host cell, may comprise one or more of these nucleic acid sequences, such as, for example, a repressor.

It is contemplated that an expression vector for a prokaryotic host cell will comprise a nucleic acid sequence that encodes a periplasmic space signal peptide. In some aspects, this nucleic acid sequence will be operatively linked to a nucleic acid sequence comprising an enzymatic peptide or polypeptide, wherein the periplasmic space signal peptide directs the expressed fusion protein to be translocated into a prokaryotic host cell's periplasmic space. Fusion proteins secreted in the periplasmic space may be obtained through simplified purification protocols compared to non-secreted fusion proteins. A periplasmic space signal peptide are usually operatively linked at or near the N-terminus of an expressed fusion protein. Examples of a periplasmic space signal peptide include the Escherichia coli ompA, ompT, and malel leader peptide sequences and the T7 caspid protein leader peptide sequence (Unger, T. F., 1997).

Mutated and/or recombinantly altered bacterium that release a peptide or polypeptide comprising an enzyme sequenceinto the environment may be for purification and/or contact of enzyme with a target chemical substrate. It is contemplated that a strain of bacteria, such as, for example, a bacteriocin-release protein mutant strain of Escherichia coli, may be used to promote release of expressed proteins targeted to the periplasm into the extracellular environment (Van der Wal, F. J. et al., 1998). In other aspects, it is contemplated that a bacterium may be transfected with an expression vector that produces a gene and/or a gene fragment product that promotes the release of a protenaceous molecule of interest from the periplasm into the extracellular environment. For example, a plasmid encoding the third topological domain of TolA has been described as promoting the release of endogenous and recombinantly expressed proteins from the periplasm (Wan, E. W. and Baneyx, F., 1998).

### D. Host Cells

Many host cells from various cell types and organisms are available and would be known to one of skill in the art. As used herein, the terms “cell,” “cell line,” and “cell culture” may be used interchangeably. All of these terms also include their progeny, which includes any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene and/or gene fragment encoded by a vector. A host cell can, and has been, used as a recipient for vectors. A host cell may be “transfected” or “transformed,” which refers to a process by which exogenous nucleic acid sequence is transferred or introduced into the host cell. A transformed cell includes the primary subject cell and its progeny. Techniques for transforming a cell include, for example calcium phosphate precipitation, cell sonication, diethylaminoethanol (“DEAE”)-dextran, direct microinjection, DNA-loaded liposomes, electroporation, gene bombardment using high velocity microprojectiles, receptor-mediated transfection, viral-mediated transfection, or a combination thereof [In “Molecular Cloning” (Sambrook, J., and Russell, D. W., Eds.) 3rd Edition, Cold Spring Harbor, N.Y.: Cold Spring Harbor Laboratory Press, 2001; In “Current Protocols in Molecular Biology” (Chanda, V. B. Ed.) John Wiley & Sons, 2002].

Once a suitable expression vector is transformed into a cell, the cell may be grown in an appropriate environment, and in some cases, used to produce a tissue or whole multicellular organism. As used herein, the terms “engineered” and “recombinant” cells or host cells are intended to refer to a cell into which an exogenous nucleic acid sequence has been introduced. Therefore, engineered cells are distinguishable from naturally occurring cells that do not contain a recombinantly introduced exogenous nucleic acid sequence. Engineered cells are thus cells having a nucleic acid sequence introduced through the hand of man. Recombinant cells include those having an introduced cDNA or genomic gene and/or a gene fragment positioned adjacent to a promoter not naturally associated with the particular introduced nucleic acid sequence, a gene, and/or a gene fragment. An enzyme or proteinaceous molecule produced from the introduced gene and/or gene fragment is referred to as a recombinant enzyme or recombinant proteinaceous molecule, respectively. All tissues, offspring, progeny or descendants of such a cell, tissue, and/or organism that comprise the transformed nucleic acid sequence thereof may be used.

Though it is possible to purify an expressed enzyme from cellular material, some embodiments disclosed herein use the properties of an enzyme composition comprising, an enzyme expressed and retained within a cell, whether naturally or through recombinant expression. In certain embodiments, an enzyme is produced using recombinant nucleic acid expression systems in the cell. Cells are known herein based on the type of enzyme expressed within the cell, whether endogenous or recombinant, so that, for example, a cell expressing an enzyme of interest would be known as an enzyme cell, a cell expressing a lipase would be known herein as a “lipase cell,” etc. Additional examples of such nomenclature include a carboxylesterase cell, an OPAA cell, a human phospholipase A₁ cell, a carboxylase cell, a cutinase cell, an aminopeptideases cell, etc., respectively denoting cells that comprise, a carboxylesterase, an OPAA, a human phospholipase A₁, a carboxylase, a cutinase, an aminopeptideases, etc.

In some embodiments, a cell comprises a bacterial cell, a fungal cell (e.g., a yeast cell), an insect cell, a plant cell, or a combination thereof. In some aspects, the cell comprises a cell wall. Contemplated enzyme that comprise within a cell walls include, but are not limited to, a bacterial cell, a fungal cell, a plant cell, or a combination thereof. In some facets, a microorganism comprises the enzyme. Examples of contemplated microorganisms include a bacterium, a fungus, or a combination thereof. Examples of a bacterial host cell that have been used with expression vectors include an Aspergillus niger, a Bacillus (e.g., B. amyloliquefaciens, B. brevis, B. licheniformis, B. subtilis), an Escherichia coli, a Kluyveromyces lactis, a Moraxella sp., a Pseudomonas (e.g., fluorescens, putida), Flavobacterium cell, a Plesiomonas cell, an Alteromonas cell, or a combination thereof. Examples of a yeast cell include a Streptomyces lividans cell, a Gliocladium virens cell, a Saccharomyces cell, or a combination thereof.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection, which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Examples of a bacterial cell used as a host cell for vector replication and/or expression include DH5a, JM109, and KC8, as well as a number of commercially available bacterial hosts such as Novablue™ Escherichia coli cells (NOVAGENE®), SURE® Competent Cells and SOLOPACK™ Gold Cells (STRATAGENE®). However, Escherichia coli cells have been the common cell types used to express both wild type and mutant forms of OPH (Dumas, D. P. et al., 1989a; Dave, K. I. et al., 1993; Lai, K. et al., 1994; Wu, C.-F. et al., 2001a). In an example, the OPH I106A/F132A/H257Y and G60A mutants have been expressed in Escherichia coli BL-21 host cells (Kuo, J. M. and Raushel, F. M., 1994; Li, W.-S. et al., 2001). In a further example, maltose-binding domain-E3 carboxylase and phosphoric triester hydrolase functional equivalents have been expressed in Escherichia coli TB1 cells (Claudianos, C. et al., 1999). In another example, the OPH mutants designated W131F, F132Y, L136Y, L140Y, H257L, L271Y, F306A, and F306Y each have been expressed in Novablue™ Escherichia coli cells (Gopal, S. et al., 2000). In an additional example, OPAA from Alteromonas sp JD6.5 has been recombinantly expressed in Escherichia coli cells (Hill, C. M., 2000). In a further example, recombinant Altermonas sp. JD6.5 OPAA has been expressed in Escherichia coli (Cheng, T.-C. et al., 1999). In a further example, the mpd gene has been recombinantly expressed in Escherichia coli, and the encoded enzyme demonstrated methyl parathion degradation activity (Zhongli, C. et al., 2001). In an additional example, a recombinant squid-type DFPase fusion protein has been expressed Escherichia coli BL-21 cells (Hartleib, J. and Ruterjans, H., 2001a). Alternatively, bacterial cells such as Escherichia coli LE392 could be used as host cells for phage viruses. Of course, one of skill in the art may select a bacterium species to express a proteinaceous molecule due to a particular desirable property. In an example, Moraxella sp. that degrades p-nitrophenol, a toxic cleavage product of parathion and methyl parathion, has been used to recombinantly express an OPH-InaV fusion protein. The resulting recombinant bacterial degrades both toxic OP compounds and their cleavage byproduct (Shimazu, M. et al., 2001b).

Examples of eukaryotic host cells for replication and/or expression of a vector include yeast cells HeLa, NIH3T3, Jurkat, 293, Cos, CHO, Saos, and PC12. In an example, OPH has been expressed in the host yeast cells of Streptomyces lividans (Steiert, J. G. et al., 1989). In another example, OPH has been expressed in host insect cells, including Spodoptera frugiperda sf9 cells (Dumas, D. P. et al., 1989b; Dumas, D. P. et al., 1990). In a further example, OPH has been expressed in the cells of Drosophila melanogaster (Phillips, J. P. et al., 1990). In an additional example, OPH has been expressed in the fungus Gliocladium virens (Dave, K. I. et al., 1994b). In a further example, the gene for human paraoxonase, PON1, has been recombinantly expressed in human embryonic kidney cells (Josse, D. et al., 2001; Josse, D. et al., 1999). In a further example, E3 carboxylase and phosphoric triester hydrolase functional equivalents have been expressed in host insect Spodoptera frugiperda sf9 cells (Campbell, P. M. et al., 1998; Newcomb, R. D. et al., 1997). In an additional example, a phosphoric triester hydrolase functional equivalent of a butyrylcholinesterase has been expressed in Chinese hamster ovary (“CHO”) cells (Lockridge, O. et al., 1997). In certain embodiments, an eukaryotic cell that may be selected for expression is a plant cell, such as, for example, a corn cell.

### E. Production of Expressed Proteinaceous Molecules

It is contemplated that any size flask or fermentor may be used to grow a tissue or organism that can express a recombinant proteinaceous molecule. In certain embodiments, bulk production of compositions with enzymatic sequences is contemplated.

In an example, a fusion protein comprising, N-terminus to C-terminus, a (His)6 polyhistidine tag, a green fluorescent protein (“GFP”), an enterokinase recognition site, and a OPH lacking the 29 amino acid leader sequence, has been expressed in Escherichia coli. The GFP sequence produced fluorescence that was proportional both the quantity of the fusion protein, and the activity of the OPH sequence. The fusion protein was more soluble than OPH expressed without the added sequences, and was expressed within the cells (Wu, C.-F. et al., 2000b; Wu, C.-F. et al., 2001a).

It is contemplated that the temperature selected may influence the rate and/or quality of recombinant enzyme production. It is contemplated that in some embodiments, expression of an enzyme may be conducted at 4° C. to 50° C., including all intermediate ranges and combinations thereof. Such combinations may include a shift from one temperature (e.g., 37° C.) to another temperature (e.g., 30° C.) during the induction of the expression of proteinaceous molecule. For example, both eukaryotic and prokaryotic expression of OPH may be conducted at temperatures 30° C., which has increased the production of enzymatically active OPH by reducing protein misfolding and inclusion body formation in some instances (Chen-Goodspeed, M. et al., 2001b; Wang, J. et al., 2001; Omburo, G. A. et al., 1992; Rowland, S. S. et al., 1991). In an additional example, prokaryotic expression of recombinant squid-type DFPase fusion protein at 30° C. also enhanced yields of active enzyme (Hartleib, J. and Ruterjans, H., 2001a). It is contemplated that fed batch growth conditions at 30° C., in a minimal media, using glycerol as a carbon source, will be suitable for expression of various enzymes.

### F. Production of Cells and Viruses

It is contemplated that any technique in the art may be used in the isolation, growth and storage of a virus, a cell, a microorganism, and a multicellular organism from which a biomolecular composition may be derived, including those where endogenously or recombinantly produce biomolecules are desired. Such techniques of cell isolation, characterization, genetic manipulation, preservation, small-scale Folid medium or liquid medium production growth, growth optimization, large (“industrial”) scale production of a biomolecule (“fermentation”), separation of a biomolecule from a cell or visa versa, etc. for various cell types (e.g., microorganisms, Eubacteria, fungi, protozoa cells, algae cells, extremophile cells, insect cells, plant cells, mammalian cells, recombinantly modified viruses or cells) are used in the art [see, for example, in “Manual of Industrial Microbiology and Biotechnology, 2^{nd} Edition (Demain, A. L. and Davies, J. E., Eds.), 1999; “Maintenance of Microorganism and Cultured Cells—A Manual of Laboratory Methods, 2^{nd} Edition” (Kirsop, B. E. and Doyle, A., Eds.), 1991; Walker, G. M. “Yeast Physiology and Biotechnology,” 1998; “Molecular Industrial Mycology Systems and Applications for Filamentous Fungi” (Leong, S. A. and Berka, R. M., Eds.), 1991; “Recombinant Microbes for Industrial and Agricultural Applications” (Murooka, Y. and Imanaka, T., Eds.), 1994; “Handbook of Applied Mycology Fungal Biotechnology Volume 4” (Arora, D. K., Elander, R. P., Mukerji, K. G., Eds.), 1992; “Genetics and Breeding of Industrial Microorganisms” (Ball, C., Ed.), 1984; “Microbiological Methods Seventh Edition” (Collins, C. H., Lyne, P. L., Grange, J. M., Eds.), 1995; “Handbook of Microbiological Media” (Parks, L. C., Ed.), 1993; Waites, M. J. et al., “Microbiology—An Introduction,” 2001; “Rapid Microbiological Methods in the Pharmaceutical Industry,” (Easter, M. C., Ed.), 2003; “Handbook of Microbiological Quality Control Pharmaceuticals and Medical Devices” (Baird, R. M., Hodges, N. A., Denyer, S. P., Eds.), 2000; “Bioreactor System Design” (Asenjo, J. A. and Marchuk, J. C., Eds.), 1995; Endress, R. “Plant Cell Biotechnology,” 1994; Slater, A. et al., “Plant Biotechnology—The genetic manipulation of plants,” 2003; “Molecular Cloning” (Sambrook, J., and Russell, D. W., Eds.), 3rd Edition, 2001; and “Current Protocols in Molecular Biology” (Chanda, V. B. Ed.), 2002.]. In embodiments wherein a cell or virus is pathogenic (e.g., pathogenic to a desirable organism) may be produced, techniques in the art may be used for handling pathogens, including identification of a pathogen, production of a pathogen, sterilizing a pathogen, attenuating a pathogen, as well as conducting cell preparation to reduce the quantity of a pathogen in non-pathogenic material [see, for example, In “Manual of Commercial Methods in Clinical Microbiology” (Truant, A. L., Ed.), 2002; “Manual of Clinical Microbiology 8^{th} Edition Volume 1” (Murray P. R., Baron, E. J., Jorgensen, J. H., Pfaller, M. A., Yolken, R. H., Eds.), 2003; “Manual of Clinical Microbiology 8th Edition Volume 2” (Murray P. R., Baron, E. J., Jorgensen, J. H., Pfaller, M. A., Yolken, R. H., Eds.), 2003; and “Biological Safety Principles and Practice 3^{rd} Edition” (Fleming, D. O. and Hunt, D. L., Eds.), 2000].

In certain embodiments, cells that endogenously or recombinantly produce a biomolecule (e.g., an enzyme) that is thermophilic, psychrophilic or mesophilic may be selected to produce a biomolecular composition for use in environments that match or overlap the conditions the biomolecule can function. It is contemplated that any enzyme for use in the embodiments may be so selected. For example, a cell or plurality of cells that produce one or more mesophilic lipolytic enzymes, psychrophilic lipophilic enzymes, and thermophilic lipolytic enzymes may be incorporated into a coating or surface treatment to confer lipolytic activity over a wide range of temperature conditions for use in temperate environmental conditions. In a further example, a cell that endogenously or recombinantly produces a thermophilic lipolytic enzyme may be selected for production of a biomolecular composition that comprises the thermophilic lipolytic enzyme. The biomolecular composition can then be incorporated into a coating or surface treatment to confer a lipolytic property that will be active in a thermophilic temperature, such as, for example, a coating for use in kitchens near hot stoves where oils and fats are heated. Examples of thermophiles that are contemplated for use are shown at the tables below.

TABLE 6
Examples of Archaea Thermophiles and Culture Sources

| | Examples of Culture |
|---|---|
| Genus (growth range) | Collection Strains |
| Acidianus (45° C.-96° C.) | DSMZ Nos. 3772, 1651 and 3191 |
| Archaeoglobus (65° C.-95° C.) | DSM 4304, 4139, 5631 and 11195 |
| Desulfurococcus (70° C.-95° C.) | DSMZ Nos. 3822, 2161 and 2162 |
| Hyperthermus (95° C.-107° C.) | DSMZ No. 5456 |
| Metallosphaera (50° C.-80° C.) | DSMZ Nos. 10039 and 5348 |
| Methanobacterium (37° C.-68° C.) | DSMZ Nos. 3387, 863, 7095, 5982, |
| | 1535, 2611, 11106, 3108, 2257, |
| | 11074, 3266 and 2956 |
| Methanococcus (35° C.-91° C.) | DSMZ Nos. 2067, 1224 and 1537 |
| Methanohalobium (50° C.-55° C.) | DSMZ Nos. 3721 and 5814 |
| Methanosarcina (30° C.-55° C.) | DSMZ Nos. 2834, 14042, 800, |
| | 13486, 2053, 12914, 3028, 4659, |
| | 1825, 2834, and 1232, ATCC 35395 |
| Methanothermus (83° C.-88° C.) | DSMZ Nos. 2088 and 3496 |
| Methanosaeta (55° C.-60° C.) | DSMZ Nos. 2139, 3013, 6752, |
| | 17206, 4774 |
| Methanothrix (35° C.-65° C.) | DSMZ Nos. 6194 |
| Pyrobaculum (74° C.-103° C.) | DSMZ Nos. 7523, 13514, 4184, |
| | 13380 and 4185 |
| Pyrococcus (70° C.-103° C.) | DSMZ Nos. 3638, 12428 and 3773 |
| Pyrodictium (80° C.-110° C.) | DSMZ Nos. 6158, 2708 and 2709 |
| Staphylothermus (65° C.-98° C.) | DSMZ Nos. 12710 and 3639 |
| Sulfolobus (55° C.-87° C.) | DSMZ Nos. 639, 7519, 6482, 5389, |
| | 1616T, 1617, 5354, 5833 and 1616 |
| Thermococcus (50° C.-98° C.) | DSMZ Nos. 11906, 12767, 12819, |
| | 10322, 11836, 2476, 10152, 12820, |
| | 10395, 11113, 5473, 10394, 10343, |
| | 9503, 12597, 12349, 5262, 12768 |
| | and 2770 |
| Thermofilum (70° C.-95° C.) | DSMZ Nos. 2475 |
| Thermoproteus (70° C.-97° C.) | DSMZ Nos. 2338, 2078 and 5263 |

TABLE 7
Examples of Gram-negative Thermophiles and Culture Sources

| | |
|---|---|
| Acetomicrobium (58-73° C.) | ATCC Nos. 43122; DSMZ Nos. |
| | 20678 and 20664 |
| Chlorobium tepidum | ATCC Nos. DSMZ No. 245, 266 |
| (55° C. to 56° C.) | and 269 |
| Chloroflexus aurantiacus | ATCC Nos. 29365 and 29366; |
| (20-66° C.) | DSMZ Nos. 635, 636, 637 |
| | and 638) |
| Desulfurella (52-57° C.) | ATCC Nos. 51451; DSMZ Nos. 5264, |
| | 10409 and 10410 |
| Dichotomicrobium (35-55° C.) | ATCC Nos. 49408; DSMZ No. 5002 |
| Fervidobacterium (40-80° C.) | ATCC Nos. 35602 and 49647 |
| Flexibacter (18-47° C.) | ATCC Nos. 23079, 23086, 23087, |
| | 23090 and 23103 |
| Isosphaera (35-55° C.) | ATCC Nos. 43644; DSMZ No. 9630 |
| Methylococcus (30-50° C.) | ATCC Nos. 19069 |
| Microscilla (30-45° C.) | ATCC Nos. 23129, 23134, |
| | 23182 and 23190 |
| Oscillatoria (56-60° C.) | ATCC Nos. 27906 and 27930 |
| Thermodesulfobacterium | DSMZ Nos. 2178, 12571, 14290, |
| (65-70° C.) | 1276 and 8975 |
| Thermoleophilum (45-70° C.) | ATCC Nos. 35263 and 35268 |
| Thermomicrobium (45-80° C.) | DSMZ No. 5159 |
| Thermonema (60-70° C.) | ATCC Nos. 43542; DSMZ Nos. 5718 |
| | and 10300 |
| Thermosipho (33-77° C.) | DSMZ No. 5309, 13481, 12029 and |
| | 6568 |
| Thermotoga (55-90° C.) | ATCC Nos. 43589, 51869, BAA-301, |
| | BAA-488 and BAA-489 |
| Thermus (70-75° C.) | ATCC Nos. 25105, 27634, 27978, |
| | 31556 and 31674 |
| Thiobacillus aquaesulis | ATCC Nos. 23642, 23645, 27977 |
| (40-50° C.) | and 43788 |

TABLE 8
Examples of Gram-positive Thermophiles and Culture Sources

| Clostridium (10° C.-65° C.) | ATCC Nos. 10000, 10092, 10132, 10388 |
|---|---|
| | and 49002 |
| Desulfotomaculum | ATCC Nos. 19858, 23193, 49208, 49756 |
| (20° C.-70° C.) | and 700205 |
| Rubrobacter (46° C.-48° C.) | ATCC No. 51242; DSMZ Nos. 5868 and |
| | 9941 |
| Saccharococcus | ATCC No. 43124; DSMZ No. 4749 |
| (68° C.-78° C.) | |
| Sphaerobacter (55° C.) | DSMZ No. 20745 |
| Thermacetogenium | DSMZ No. 12270 |
| (55° C.-58° C.) | |
| Thermoanaerobacter | ATCC Nos. 31936, 31960, 33488, 35047 |
| (35° C.-78° C.) | and 49915 |
| Thermoanaerobium | DSMZ Nos. 7040, 1457, 9766, 9003 and |
| (45° C.-75° C.) | 9769 |

Examples of psychrophiles and culture sources include Moritella (ATCC Nos. 15381 and BAA-105; DSMZ No. 14879), Leifsonia aurea (DSMZ No. 15303, CIP No. 107785, MTCC No. 4657), and Methanococcoides burtonii (DSM No.: 6242). Examples of halophiles and culture sources include Halobacterium (DSMZ Nos. 3754 and 3750), Halococcus (DSMZ Nos. 14522, 1307, 5350 and 8989), Haloferax (DSMZ Nos. 4425, 4427, 1411 and 3757), Halogeometricum (DSMZ No. 11551; JCM No. 10706), Haloterrigena (DSMZ Nos. 11552 and 5511), Halorubrum (DSMZ Nos. 10284, 5036, 1137, 3755, 14210 and 8800), and Haloarcula (ATCC 43049, DSMZ Nos. 12282, 4426, 6131, 3752, 11927, 8905 and 3756).

In other embodiments, cells that endogenously or recombinantly produce a petroleum lipolytic enzyme may be selected to produce a biomolecular composition for use in aiding removal of petroleum lipids from a surface having a coating or surface treatment. Examples of such microorganism genera and strains contemplated for use in production of a petroleum lipolytic enzyme (e.g., a cell-based particulate material comprising a petroleum lipolytic enzyme) include Azoarcus [DSMZ Nos. 12081, 14744, 6898, 9506 (sp. strain T) and 15124], Blastochloris (DSMZ Nos. 133, 134, 136, 729 and 13255 (ToP1)), Burkholderia (DSMZ Nos. 9511, 50341, 13243, 13276 and 11319), Dechloromonas (ATCC No. 700666; DSMZ No. 13637), Desulfobacterium [ATCC Nos. 43914, 43938 and 49792; DSMZ: 6200 (cetonicum strain Hxd3)], Desulfobacula (ATCC No. 43956; DSMZ Nos. 3384 and 7467), Geobacter [DSMZ Nos. 12179, 13689 (grbiciae TACP-2T), 13690 (grbiciae TACP-5), 7210 (metallireducens GS15), 12255, and 12127], Mycobacterium (ATCC Nos. 10142, 10143, 11152, 11440 and 11564), Pseudomonas (ATCC Nos. 10144, 10145, 10205, 10757 and 27853), Rhodococcus (ATCC Nos. 10146, 11048, 12483, 12974 and 14346), Sphingomonas (DSMZ Nos. 7418, 10564, 1805, 13885 and 6014), Thauera [DSMZ Nos. 14742, 12138, 12266, 14743, 12139 and 6984 (aromatica K172)], Vibrio (ATCC Nos. 11558, 14048, 14126, 14390 and 15338), or a combination thereof. Examples of microorganism strains for petroleum lipolytic enzyme production, and target substrates, include Azoarcus sp. strain EB1 (ethylbenzene), Azoarcus sp. strain T (toluene, m-xylene), Azoarcus tolulyticus Td15 (toluene, m-xylene), Azoarcus tolulyticus To14 (toluene), Blastochloris sulfoviridis ToP1 (toluene), Burkholderia sp. strain RP007 (naphthalene phenanthrene), Dechloromonas sp. strain JJ (benzene, toluene), Dechloromonas sp. strain RCB (benzene, toluene), Desulfobacterium cetonicum (toluene), Desulfobacterium cetonicum strain AK-01 (C13-C18 alkanes), Desulfobacterium cetonicum strain Hxd3 (C12-C₂₀ alkanes, 1-hexadecene), Desulfobacterium cetonicum strain mXyS1 (toluene, m-xylene, m-ethyltoluene, m-cymene), Desulfobacterium cetonicum strain NaphS2 (naphthalene), Desulfobacterium cetonicum strain oXyS1 (toluene o-xylene, o-ethyltoluene), Desulfobacterium cetonicum strain Pnd3 (C₁₄-C₁₇ alkanes, 1-hexadecene), Desulfobacterium cetonicum strain PRTOL1 (toluene), Desulfobacterium cetonicum strain TD3 (C6-C16 alkanes), Desulfobacula toluolica To12 (toluene), Geobacter grbiciae TACP-2T (toluene), Geobacter grbiciae TACP-5 (toluene), Geobacter 7210 metallireducens GS15 (toluene), Mycobacterium sp. strain PYR-1 (anthracene, benzopyrene, fluoranthene, phenanthrene, pyrene, 1-nitropyrene), Pseudomonas putida NCIB9816 (naphthalene), Pseudomonas putida OUS82 (naphthalene, phenanthrene, various cyclic hydrocarbons), Pseudomonas sp. strain C18 (dibenzothiophene, naphthalene phenanthrene), Pseudomonas sp. strain EbN1 (ethylbenzene, toluene), Pseudomonas sp. strain HdN1 (C14-C20 alkanes), Pseudomonas sp. strain HxN1 (C6-C8 alkanes), Pseudomonas sp. strain M3 (toluene, m-xylene), Pseudomonas sp. strain mXyN1 (toluene, m-xylene), Pseudomonas sp. strain NAP-3 (naphthalene), Pseudomonas sp. strain OcN1 (C8-C12 alkanes), Pseudomonas sp. strain PbN1 (ethylbenzene, propylbenzene), Pseudomonas sp. strain pCyN1 (p-Cymene, toluene, p-ethyltoluene), Pseudomonas sp. strain pCyN2 (p-Cymene), Pseudomonas sp. strain T3 (toluene), Pseudomonas sp. strain ToN1 (toluene), Pseudomonas sp. strain U2 (naphthalene), Pseudomonas stutzeri AN10 (naphthalene, 2-methylnaphthalene), Rhodococcus sp. strain 124 (indene, naphthalene, toluene), Sphingomonas paucimobilis var. EPA505 (anthracene, fluoroanthene, naphthalene, phenanthrene, pyrene), Thauera aromatica K172 (toluene), Thauera aromatica T1 (toluene), Vibrio sp. strain NAP-4 (naphthalene), or a combination thereof.

### G. Cell-Based Biomolecular Compositions

After production of a living cell, the cell may be used as a biomolecular composition. Such a biomolecular composition is known herein as a “crude cell preparation”. A crude cell preparation comprises a desired biomolecule (e.g., an active biomolecule such as a lipase), within or otherwise in contact with a cell and/or cellular debris. In certain aspects, it is contemplated that the total content of desired biomolecule may range from 0.0001% to 99.9999% of a crude cell preparation, including all intermediate ranges and combinations thereof, by volume or dry weight, depending upon factors such as expression efficiency of the biomolecule in the cell and the amount of processing and/or purification steps. A higher content of desired biomolecule in the biomolecular composition is selected in specific embodiments when conferring activity to a surface treatment such as a coating. But, in certain embodiments, the biomolecular composition comprises cellular components, particularly cell wall and/or cell membrane material, to provide material that may be protective to the biomolecule, enhances the particulate nature of the biomolecular composition, or a combination thereof. Thus, the biomolecular composition may comprise 0.0001% to 99.9999% of cellular components, including all intermediate ranges and combinations thereof, by volume or dry weight. However, in certain embodiments, lower ranges of cellular components are used, as the biomolecular composition would therefore comprise a greater percentage of a desired biomolecule.

In embodiments wherein the cellular material is derived from a microorganism, such as through expression of the biomolecule by a microorganism, the biomolecular composition is known herein as a “microorganism based particulate material”. The association of a biomolecule with a cell or cellular material is generally produced through endogenous expression, expression due to recombinant engineering, or a combination thereof. In some embodiments, a crude cell preparation comprises a biomolecule partly or whole encapsulated by a cell membrane and/or cell wall, whether naturally so and/or through recombinant engineering. Such a biomolecule (e.g., the active biomolecule) encapsulated within or as a part of a cell wall and/or cell membrane is referred to herein as a “whole cell material” or “whole cell particulate material.”

An embodiment of the cell-based particulate material is the material in the form of a “whole cell material,” which refers to particulate material resembling an intact living cell upon microscopic examination, in contrast to cell fragments of varying shape and size. It is contemplated that such whole cell particulate material will encapsulate an expressed biomolecule (e.g., an enzyme) located in and/or internal to a cell wall and/or cell membrane. In certain aspects, the encapsulation of a biomolecule by a whole cell particle may provide greater protection of the biomolecule from a coating component (e.g., a solvent, a binder, an additive), a coating related chemical reaction (e.g., thermosetting film formation), a potentially damaging agent a coating and/or film may contact (e.g., a chemical, a solvent, a detergent, etc.), or a combination thereof, relative to a biomolecule located on the external surface of a cell or otherwise not comprised within and/or encapsulated by a cell wall and/or cell membrane. Any preparation of a cell will comprise a certain percentage of cell fragments, which comprise pieces of a cell wall, cell membrane, and other cell components (e.g., an expressed biomolecule). The whole cell particulate material will comprise 50% to 100%, including all intermediate ranges and combinations thereof of whole cell material. The percentage of whole cell material and cell fragments may be determined by any applicable technique in the art such as microscopic examination, centrifugation, etc, as well as any technique described herein for determining the properties of a pigment, extender, or other particulate material either alone or comprises in a coating. It is contemplated that in some aspects, cell fragments may be used as cell-based particulate material. The cell fragment cell-based particulate material will comprise 50% to 100%, including all intermediate ranges and combinations thereof of cell fragment material.

In some embodiments, a multicellular organism (e.g., a plant) may undergo a processing step wherein one or more cells are physically, chemically, and/or enzymatically separated to produce a material with desired particulate properties for a coating or other surface treatment formulation. In certain embodiments, cells and/or cell components may be separated using a disrupting step, described herein. As microorganisms are generally unicellular or oligocellular in nature, they are used in many embodiments, as it is contemplated that the number of processing steps used to prepare a cell-based particulate material from such an organism will be fewer than for a cell from a multicellular organism. For example, a particulate material for a coating or other surface treatment may be selected for properties such as ease of dispersal, particle size, particle shape, etc. It is contemplated that a microorganism may be selected for cell shape, cell size, ease of dispersal, due to poor affinity for other cells relative to a cell embedded in a multicellular organism, or a combination thereof, to produce a cell-based particulate material with desired particulate material properties using fewer processing steps and/or with greater ease than a multicellular organism.

In certain embodiments, a cell-based particulate materialmay comprise various cellular components (e.g., cell wall material, cell membrane material, nucleic acids, sugars, polysaccharides, peptides, polypeptides, proteins, lipids, etc.). Such cell or virus biomolecule components have been described (see, for example, CRC Handbook of Microbiology. Volume 1, bacteria; Volume 2, fungi, algae, protozoa, and viruses; Volume 3, microbial compositions: amino acids, proteins, and nucleic acids; Volume 4, microbial compositions: carbohydrates, lipids, and minerals; Volume 5, microbial products; Volume 6, growth and metabolism; Volume 7, microbial transformation; Volume 8. toxins and enzymes; Volume 9, pt. A. antibiotics—Volume. 9, pt. B. antimicrobial inhibitors; 1977). In certain embodiments, the cell-based particulate material comprises a cell wall and/or cell membrane material, to enhance the particulate nature of the cell-based particulate material. However, in many aspects the cell-based particulate material comprises cell wall material, as it is contemplated that the cell wall is the dominant cellular component for conferring particulate material properties such as shape, size, and insolubility.

Depending upon the type of processing used various cell components may be partly or fully removed from the organism to produce a cell-based particulate material. In particular, a processing step wherein a cell is contacted with a liquid (e.g., an organic liquid) is contemplated to dissolve many cell components. Removal of the solvent would thereby remove (“extract”) the dissolved cell components from the particulate matter. However, it is additionally contemplated that a large biomolecule, particularly a polymer that comprises a cell wall, such as peptidoglycan, teichoic acid, lipopolysacharide or a combination thereof, will be resistant to extraction with a non-aqueous or aqueous solvent, and thus be retained as a component of the particulate matter. In particular embodiments, it is contemplated that a biomolecule of extremely large size, such as greater than 1,000 kDa molecular mass, will be retained in the particulate matter. Further, it is contemplated that in certain embodiments, greater than 50% of the dry weight of such particulate matter will comprise a biomolecule of extremely large size and/or cell wall polymers after processing.

It is contemplated that a large biomolecule, particularly a cell wall polymer, will be at or near the interface of the particulate matter and the external environment. As this interface is primary area of contact between the particulate matter and other coating or other surface treatment components, it is contemplated that a biomolecule will contribute to the properties of the particulate matter produced from a cell used in a coating or other surface treatment. Examples of such properties include the size range of particulate matter, the shape of the particulate matter, the solubility of the particulate matter, the permeability and/or impermeability of the particulate matter to a chemical, the chemical reactivity of the particulate matter, or a combination thereof. It is also contemplated that a chemical moiety of the biomolecule at the interface of the particulate matter and the external environment may chemically react with a second coating or other surface treatment component. In certain embodiments, such reactions may be desirable, such as, for example, the chemical crosslinking of a cell-based particulate material to a binder in a thermosetting coating or surface treatment. By participating in such crosslinking reactions, it is contemplated that a cell-based particulate material may be selected for use as a binder in a coating or surface treatment.

In addition to the biomolecules described above that are contemplated as contributing to the particulate nature and/or potential chemical reactivity of a cell-based particulate material, such a composition may comprise other desirable biomolecules (e.g., a colorant, an enzyme, an antibody, a receptor, a transport protein, structural protein, an ligand, a prion) that may confer desirable properties to a surface treatment. Such biomolecules may be an endogenously produced cell component, or a product of expression of a recombinant nucleic acid in the virus or cell [see, for example, “Molecular Cloning,” 2001; and “Current Protocols in Molecular Biology,” 2002].

### H. Processing of Cells and Expressed Biomolecules

After production of a biomolecule by a living cell, the composition comprising the biomolecule may undergo one or more processing steps to prepare a biomolecular composition. Examples of such steps include concentrating, drying, applying physical force, extracting, resuspending, controlling temperature, permeabilizing, disrupting, chemically modifying, encapsulating, enzyme purification, immobilizing, or a combination thereof. Various embodiments of a biomolecular composition are contemplated after one or more such processing steps. However, it is further contemplated that each processing step may increase economic costs and/or reduce total biomolecule yield, so that embodiments comprising fewer steps may reduce costs. It is further contemplated that the order of steps may be varied and still produce a biomolecular composition.

It is contemplated that a biomolecule prepared as a crude cell preparation (e.g., a whole cell particulate material) may have greater stability than a preparation wherein the biomolecule has been substantially separated from a cell membrane and/or cell wall. It is further contemplated that a biomolecule prepared as a crude cell preparation, wherein the biomolecule is localized between the cell wall and cell membrane and/or within the cell so that the cell wall separates the biomolecule from the extracellular environment, may have greater stability than a preparation wherein the biomolecule has been substantially separated from a cell membrane and/or cell wall.

1. Sterilization/Attenuation

A processing step may comprise sterilizing an biomolecular composition. Sterilizing (“inactivating”) kills living matter (e.g., a cell, a virus), while attenuation reduces the virulence of living matter. A sterilizing and/or attenuating step may be desirable as continued post expression growth of a cell and/or a contaminating organism may detrimentally affect the composition. For example, one or more properties of a coating or other surface treatment may be undesirably altered by the presence of a living organism. Additionally, sterilizing reduces the ability of a living recombinant organism to be introduced into the environment, when such an event is not desired. A cell-based particulate material wherein the majority of material by dry or wet weight or volume has been sterilized or attenuated, is known herein as a “sterilized cell-based particulate material” or “attenuated cell-based particulate material,” respectively.

In certain embodiments, it contemplated that sterilization or attenuation may be accomplished in a surface treatment (e.g., a coating) by contact with biologically detrimental surface treatment components such as solvents or chemically reactive surface treatment components (e.g., a binder). In further embodiments, sterilizing or attenuation of a cell-based particulate material or a surface treatment comprising such a material may be accomplished by any method known in the art, and are commonly applied in the food, medical, and pharmaceutical arts to sterilize or attenuate pathogenic microorganisms [see, for example, “Food Irradiation: Principles and Applications,” 2001; “Manual of Commercial Methods in Clinical Microbiology” (Truant, A. L., Ed.), 2002; “Manual of Clinical Microbiology 8th Edition Volume 1” (Murray P. R., Baron, E. J., Jorgensen, J. H., Pfaller, M. A., Yolken, R. H., Eds.), 2003; “Manual of Clinical Microbiology 8th Edition Volume 2” (Murray P. R., Baron, E. J., Jorgensen, J. H., Pfaller, M. A., Yolken, R. H., Eds.), 2003; and “Biological Safety Principles and Practice 3 dEdition” (Fleming, D. O. and Hunt, D. L., Eds.), 2000]. Examples of sterilizing or attenuating may include contacting the living matter with a toxin, irradiating the living matter, heating the living matter above a temperature suitable for life (e.g., 100° C. in many cases, more for temperature extremophiles), or a combination thereof. In some embodiments sterilizing or attenuating comprises irradiating the living matter, as radiation generally does not leave a toxic residue, and is not contemplated to detrimentally affect the stability of a desired biomolecule (e.g., a colorant, an enzyme) that might be present in the cell-based particulate material, to the same degree as other sterilizing or attenuating techniques (e.g., heating). Examples of radiation include infrared (“IR”) radiation, ionizing radiation, microwave radiation, ultra-violet (“UV”) radiation, particle radiation, or a combination thereof. Particle radiation, UV radiation and/or ionizing radiation are contemplated in some embodiments, and particle radiation is contemplated for some facets. Examples of particle radiation include alpha radiation, electron beam/beta radiation, neutron radiation, proton radiation, or a combination thereof.

The pathogenicity of a cell or virus may be reduced or eliminated through genetic alteration (e.g., an attenuated virus with reduced pathogenicity, infectivity, etc.), processing techniques such as partial or complete sterilization and/or attenuation using techniques in the art (e.g., heat treatment, irradiation, contact with chemicals), passage of a virus through cell not typically a host cell for the virus, or a combination thereof, and such a cell or virus is used in some facets. In many embodiments, the majority (e.g., 50% to 100%, including all intermediate ranges and combinations thereof) of the cell-based particulate material has been sterilized and/or attenuated, with 100% or as close to 100% as is practically accomplishable, selected for specific facets.

However, in alternative embodiments, it is contemplated that partly or non-sterilized or attenuated cell-based particulate material will be suitable for a temporary coating (e.g., a non-film forming coating) or other temporary surface treatment. In particular aspects, the damage produced by living cells or viruses in a coating, film or other surface treatment may make the composition more suitable for use as a temporary coating or other surface treatment. For example, the cell-based particulate material may reduce the durability of the coating, film or other surface treatment over time (e.g., degrade a binder molecule), enhance ease of removal of the coating, film or other surface treatment (e.g., reduce resistance to a solvent), etc.

2. Concentrating

A processing step may comprise concentrating a biomolecular composition. As used herein, “concentrating” refers to any process wherein the volume of a composition is reduced. Often, undesired components that comprise the excess volume are removed; the desired composition is localized to a reduced volume, or a combination thereof.

For example, it is contemplated that a concentrating step may be used to reduce the amount of a growth and/or expression medium component from a composition. It is contemplated that nutrients, salts and other chemicals that comprise a biological growth and/or expression medium may be unnecessary and/or unsuitable in a composition, and reducing the amount of such compounds is contemplated. A growth medium may promote undesirable microorganism growth in a composition, while salts or other chemicals may undesirably alter the formulation of a coating or other surface treatment.

Concentrating a biomolecular composition (e.g., cell-based particulate material) may be by any method known in the art, including, for example, washing, filtrating, a gravitational force, a gravimetric force, or a combination thereof. An example of a gravitational force is normal gravity. An example of a gravimetric force is the force exerted during centrifugation. Often a gravitational or gravimetric force is used to concentrate a biomolecular composition from undesired components that are retained in the volume of a liquid medium. After desired biomolecules (e.g., cell based particulate materials) are localized to the bottom of a centrifugation devise, the media may be removed via such techniques as decanting, aspiration, etc.

3. Drying

In additional embodiments, the biomolecular composition is dried. Such a drying step may remove undesired liquids, in particular from a cell-based particulate material. Examples of drying include freeze-drying, lyophilizing, or a combination thereof. In some aspects, a cryoprotectant may be added to the biomolecular composition during a drying step (e.g., lyophilizing). In certain embodiments, it is contemplated that a drying step may enhance the particulate nature of the material. For example, reduction of a liquid in the cell-based particulate material may reduce the tendency of particles of the material to adhere to each other (e.g., agglomerate, aggregate), or a combination thereof. It is also contemplated that in some aspects, the particulate material may be in a form (e.g., a powder) sufficiently liquid free (“dry”) that it is suitable for convenient storage at ambient conditions without need for desiccation.

4. Physical Force

It is contemplated that an application of physical force (e.g., grinding, milling, shearing) may enhance the particulate nature of the material by converting multicellular material (e.g., a plant) into oligocellular and/or unicellular material, or convert oligocellular material into unicellular material. Such an application of physical force generally will be referred to as “milling” herein, particularly the claims. Further, the average particle size may be reduced to a desired range, including the conversion of cells into disrupted cells and/or cell debris. It is also contemplated that such physical force may produce a powder form of a cell-based particulate material. Physical force may also be used in processing steps dealing with purified or semi-purified biomolecules (e.g., enzymes).

5. Extraction

It is contemplated that a biomolecule may be removed by extraction of a biomolecular composition (e.g., a cell-based particulate material). For example, it is contemplated that a lipid and/or an aqueous component of a cell-based particulate material may be partly or fully removed by extraction with appropriate solvents. Such extraction may be desirable to dry the cell-based particulate material by removal of liquid (e.g., water, lipids), remove of a biotoxin, sterilize/attenuate living material in the composition, disrupt and/or permeablize a cell, alter the physical and/or chemical characteristics of the cell-external environment interface, or a combination thereof. For example, lipids such as phospholipids are often present at or within a cell wall and/or membrane, and an extraction step may partly or fully remove those lipids likely to chemically react with other surface treatment components. Additionally, such an extraction of surface lipids may alter (e.g., increase or decrease) the hydrophobicity or hydrophilicity of a cell-based particulate material to enhance its suitability (e.g., disperability) for a specific coating or other surface treatment.

6. Resuspending

A purification step may comprise resuspending a precipitated composition comprising biomolecule (e.g., a desired enzyme) from cell debris. In certain embodiments, a composition comprising a coating and an enzyme prepared by the following steps: obtaining a culture of cells that express the enzyme; concentrating the cells and removing the culture media; disrupting the cell structure; drying the cells; and adding the cells to the coating. In some aspects, the composition is prepared by the additional step of suspending the disrupted cells in a solvent prior to adding the cells to the coating.

In certain aspects, the composition is prepared by adding the cell culture powder to glycerol, admixing with glycerol and/or suspending in glycerol. In other facets, the glycerol is at a concentration of about 50%. In specific facets, the cell culture powder comprised in glycerol at a concentration of about 3 mg of the milled powder to 3 ml of 50% glycerol. In certain facets, the composition is prepared by adding the powder comprised in glycerol to the paint at a concentration of about 3 ml glycerol comprising powder to 100 ml of paint. The powder may also be added to a liquid component such as glycerol prior to addition to the paint. The numbers are exemplary only and do not limit the use. The concentration was chosen merely to be compatible with the amount of substance that can be added to one example of paint without affecting the integrity of the paint itself. Any compatible amount may used.

A processing step may comprise resuspending the composition comprising a biomolecular composition (e.g., a cell-based particulate material). It is contemplated that the material to be resuspended will have undergone a prior processing step, such as concentration (e.g., precipitation), drying, extraction, etc., and is resuspended into a form suitable for storage, further processing, and/or addition to a coating or other surface treatment. In certain aspects, the resuspension medium is a liquid component of a coating or other surface treatment described herein, a cryopreservative (“cryoprotector”), a xeroprotectant, or a combination thereof. A cryopreservative is a substance, typically a liquid, that reduces the ability of a cell wall or cell membrane to rupture, particularly during a freezing and thawing process, while a xeroprotectant is a substance, typically a liquid, that reduces damage to a composition (e.g., a desirable biomolecular composition), during a drying process (e.g., a drying processing step, physical film formation). In some embodiments, a cryopreservative, a xeroprotectant, or a combination thereof, may be used as an additive to a coating or other surface treatment. Examples of a cryopreservative include glycerol, dimethyl sulfoxide (“DMSO”), a protein (e.g., an animal serum albumin), a sugar of 4 to 10 carbons (e.g., sucrose), or a combination thereof. Examples of a xeroprotectant include glycerol, a glycol such as a polyethylene glycol (e.g., PEG₈₀₀₀), a mineral oil, a bicarbonate (e.g., ammonium bicarbonate), DMSO, a sugar of 4 to 10 carbons (e.g., trehalose), or a combination thereof. Often, a cryopreservative and/or a xeroprotectant is in an aqueous liquid, and may comprise a pH buffer (e.g., a phosphate buffer). A substance (e.g., a cryopreservative, a xeroprotectant) included as part of a surface treatment with or as part of biomolecular composition that may alter the physical (e.g., hydrophobicity, hydrophilicity, dispersal of particulate material, etc.) or chemical properties (e.g., reactivity with a surface treatment component) of a surface treatment, and a surface treatment's formulation may be improved using the techniques described herein or the art to account for these additional coating or other surface treatment components. In certain embodiments, the amount of cryopreservative and/or a xeroprotectant will comprise 0.000001% to 80%, including all intermediate ranges and combinations thereof, of a biomolecular composition. In specific facets, a biomolecular composition, a cryopreservative and/or a xeroprotectant may comprise 0.000001% to 66% a glycerol or a glycol (e.g., a polyethylene glycol), including all intermediate ranges and combinations thereof. In other embodiments, a biomolecular composition, a cryopreservative and/or a xeroprotectant may comprise 0.000001% to 10% DMSO, including all intermediate ranges and combinations thereof. In further embodiments, a biomolecular composition, a surface treatment, a cryopreservative and/or a xeroprotectant may comprise 0.000001 M to 1.5 M bicarbonate, including all intermediate ranges and combinations thereof.

7. Temperatures

It is contemplated that in some embodiments, a processing step may comprise maintaining a biomolecular composition (e.g., a composition comprising an enzyme) at a temperature at or less than the optimum temperature for the activity of a living organism and/or enzyme that may detrimentally affect an enzyme. For example, often 37° C. is the maximum temperature for the processing of a human biomolecule (e.g., an enzyme). Thus temperatures at or less than 37° C. are contemplated in such aspects, during processing of materials derived from a human cell. Controlling the range of temperatures a biomolecular composition is exposed to during processing can be modified accordingly for thermophiles, a mesophiles, or a psychrophile derived biomolecular composition.

8. Permeabilization/Disruption

In some aspects, a biomolecular composition comprises a cell preparation (e.g., crude cell, whole cell, etc.) wherein the cell membrane and/or cell wall has been altered through a permeabilizing process, a disruption process, or a combination thereof. An example of such an altered cell preparation includes crude cells, disrupted cells, whole cells, permeabilized cells, or a combination thereof. As used herein, a “disrupted cell” is a cell preparation wherein the cell membrane and/or cell wall has been altered through a disruption process. As used herein, a “permeabilized cell” is a cell preparation wherein the cell membrane and/or cell wall has been altered through a permeabilizing process. Permeabilization and/or disruption may promote the separation of cells, reduce the average particle size of the material, allow greater access to a biomolecule in a cell (e.g., to promote ease of extraction), or a combination thereof.

A processing step may comprise a permeabilizing step, wherein a cell is contacted with a permeabilizing agent such as DMSO, ethylenediaminetetraacetic acid (“EDTA”), tributyl phosphate, or a combination thereof. A permeabilizing step may increase the mass transport of a substance (e.g., a substrate) into the interior of a cell, where an enzyme localized inside the cell can catalyze a chemical reaction with the substrate. (Martinez, M. B. et al., 1996; Martinez, M. B. et al., 2001; Hung, S.-C. and Liao, J. C., 1996). Cell permeabilizing using EDTA has been described (Leduc, M. et al., 1985).

In some embodiments, a processing step comprises disrupting a cell. A cell may be disrupted by any method known in the art, including, for example, a chemical method, a mechanical method, a biological method, or a combination thereof. Examples of a chemical cell disruption method include suspension in a solvent for certain cellular components. In specific facets, such a solvent may comprise an organic solvent (e.g., acetone), a volatile solvent, or a combination thereof. In a particular facet, a cell may be disrupted by acetone (Wild, J. R. et al., 1986; Albizo, J. M. and White, W. E., 1986). In certain facets, the cells are disrupted in a volatile solvent for ease in evaporation. Examples of a mechanical cell disruption method include pressure (e.g., processing through a French press), sonication, mechanical shearing, or a combination thereof. An example of a pressure cell disruption method includes processing through a French press. Examples of a biological cell disruption method include contacting the cell with one or more proteins/polypeptides that are known to possess such disrupting activity including porins and enzymes such as a lysozyme, as well as contact/cell infection with a virus that weakens, damages, and/or permeabilizes a cell membrane, cell wall or combination thereof. Cell-based particulate materialcomprising cells and/or cellular components may be homogenized, sheared, undergo one or more freeze thaw cycles, be subjected to enzymatic and/chemical digestion of cellular materials (e.g., cell walls, sugars, etc.), undergo extraction with organic or aqueous solvents, etc., to weaken interactions between the cellular materials. A processing step may comprise sonicating a composition. Other disrupting and drying will be done by freeze-drying with a reduced or absent cryoprotector (e.g., a sugar).

9. Chemical Modification

In certain embodiments, a biomolecular composition (e.g., a cell based particulate material) may be chemically modified for a specific physical (e.g., hydrophobicity, hydrophilicity, dispersal of particulate material, etc.) or chemical properties (e.g., reactivity with a surface treatment component) to enhance suitability in a coating or other surface treatment. In embodiments wherein a cell based particulate material is used, it is contemplated that such chemical (e.g., organic chemistry) modification will primarily affect a cell-external environment interface. Such modifications include for example, acylatylation, amination, hydroxylation, phosphorylation, methylation, adding a detectable label such as a fluorescein isothiocyanate, covalent attachment of a poly ethylene glycol, a derivation of an amino acid by a sugar moiety, a lipid, a phosphate, or a farnysyl group; or a combination thereof, as well as others in the art [see, Greene, T. W. and Wuts, P. G. M. “Productive Groups in Organic Synthesis,” Second Edition, pp. 309-315, John Wiley & Sons, Inc., USA, 1991; and co-pending U.S. patent application Ser. No. 10/655,345 “Biological Active Coating Components, Coatings, and Coated surfaces, filed Sep. 4, 2003; in “Molecular Cloning,” 2001; “Current Protocols in Molecular Biology,” 2002]. Additional modifications, particularly those more suited for purified enzymes are described herein.

10. Encapsulation

Additionally, it is contemplated that a biomolecular composition may be encapsulated using a microencapsulation technique. Such encapsulation may enhance or confer the particulate nature of the biomolecular composition, provide protection to the biomolecular composition, increase the average particle size to a desired range, allow release of a cellular component (e.g., a biomolecule) from the encapsulating material, alter surface charge, hydrophobicity, hydrophilicity, solubility and/or disperability of the particulate material, or a combination thereof. Examples of microencapsulation (e.g., microsphere) compositions and techniques are described in Wang, H. T. et al., J. of Controlled Release 17:23-25, 1991; and U.S. Pat. Nos. 4,324,683, 4,839,046, 4,988,623, 5,026,650, 5153,131, 6,485,983, 5,627,021 and 6,020,312.

11. Other Processing Steps/Enzyme Purification

In other embodiments, a proteinaceous molecule may be a purified a proteinaceous molecule. A “purified proteinaceous molecule” as used herein refers to any proteinaceous molecule removed in any degree from other extraneous materials (e.g., cellular material, nutrient or culture medium used in growth and/or expression, etc). In certain aspects, removal of other extraneous material may produce a purified proteinaceous molecule (e.g., a purified enzyme) wherein its concentration has been enhanced 2- to 1,000,000-fold or more, including all intermediate ranges and combinations thereof, from its original concentration in a material (e.g., a recombinant cell, a nutrient or culture medium, etc). In other embodiments, a purified proteinaceous molecule may comprise 0.001% to 100%, including all intermediate ranges and combinations thereof of a composition comprising a proteinaceous molecule. The degree or fold of purification may be determined using any method known to those of skill in the art or described herein. For example, it is contemplated that techniques such as measuring specific activity of a fraction by an assay described herein, relative to the specific activity of the source material, or fraction at an earlier step in purification, may be used.

Some techniques for preparation of a proteinaceous molecule are described herein. However, it is contemplated that one or more additional methods for purification of biologically produced molecule(s) (e.g., ammonium sulfate precipitation, ultrafiltration, polyethyleneglycol suspension, hexanol extraction, methanol precipitation, Triton X-100 extraction, acrinol treatment, isoelectric focusing, alcohol treatment, acid treatment, acetone precipitation, etc.) that are known in the art or described herein may be used to obtain a purified proteinaceous molecule [Azzoni, A. R. et al., 2002; In “Current Protocols in Molecular Biology” (Chanda, V. B. Ed.) John Wiley & Sons, 2002; In “Current Protocols in Nucleic Acid Chemistry” (Harkins, E. W. Ed.) John Wiley & Sons, 2002; In “Current Protocols in Protein Science” (Taylor, G. Ed.) John Wiley & Sons, 2002; In “Current Protocols in Cell Biology” (Morgan, K. Ed.) John Wiley & Sons, 2002; In “Current Protocols in Pharmacology” (Taylor, G. Ed.) John Wiley & Sons, 2002; In “Current Protocols in Cytometry” (Robinson, J. P. Ed.) John Wiley & Sons, 2002; In “Current Protocols in Immunology” (Coico, R. Ed.) John Wiley & Sons, 2002; In “Methods and Molecular Biology, Volume 109 Lipase and Phospholipase Protocols.” (Mark Doolittle and Karen Reue, Eds.), 1999; pancreatic lipase via recombinant expression in a baculoviral system in “Methods and Molecular Biology, Volume 109 Lipase and Phospholipase Protocols.” (Mark Doolittle and Karen Reue, Eds.), 1999; In “Lipases their Structure, Biochemistry and Application” (Paul Woolley and Steffen B. Peterson, Eds.), 1994; Brockerhoff, Hans and Jensen, Robert G. “Lipolytic Enzymes,” 1974; “Lipases” (Borgstrom, B. and Brockman, H. L., Eds), 1984; In “Lipases and Phospholipases in Drug Development from Biochemistry to Molecular Pharmacology.” (Müller, G. and Petry, S. Eds.), 2004]. A biological material comprising a proteinaceous molecule may be homogenized, sheared, undergo one or more freeze thaw cycles, be subjected to enzymatic and/chemical digestion of cellular materials (e.g., cell walls, sugars, etc), undergo extraction with organic or aqueous solvents, etc, to weaken interactions between the proteinaceous molecule and other cellular materials and/or partly purify the proteinaceous molecule. A processing step may comprise sonicating a composition comprising an enzyme.

Cellular materials may be further fractionated to separate a proteinaceous molecule from other cellular components using chromatographic e.g., affinity chromatography (e.g., antibody affinity chromatography, lectin affinity chromatography), fast protein liquid chromatography, high performance liquid chromatography “HPLC”), ion-exchange chromatography, exclusion chromatography; or electrophoretic (e.g., polyacrylamide gel electrophoresis, isoelectric focusing) methods. It is contemplated that a proteinaceous molecule may be precipitated using antibodies, salts, heat denaturation, centrifugation and the like. A purification step may comprise dialyzing a composition comprising an enzyme from cell debris. For example, heparin-Sepharose chromatography has been used to enhance purification of lipolytic enzymes such as diacyglycerol lipase, triacylglycerol lipase, lipoprotein lipase, phospholipase A₂, phospholipase C, and phospholipase D [see for example, in “Methods and Molecular Biology, Volume 109 Lipase and Phospholipase Protocols.” (Mark Doolittle and Karen Reue, Eds.), pp. 133-143, 1999].

For example, the molecular weight of a proteinaceous molecule can be calculated when the sequence is known, or estimated when the approximate sequence and/or length is known. SDS-PAGE and staining (e.g., Coomassie Blue) has been commonly used to determine the success of recombinant expression and/or purification of OPH, as described (Kolakowski, J. E. et al., 1997; Lai, K. et al., 1994).

In certain embodiments, an enzyme may be in the form of a crystal. In other aspects, one or more enzyme crystals may be cross-linked for from a CLEC (Hoskin, F. C. G. et al., 1999; Lalonde, J. J. et al., 1995; Persichetti, R. A., 1996)).

12. Immobilization

Immobilization refers to attachment (i.e., by covalent or non-covalent interactions) of an enzyme to a solid support (“carrier”) or crosslinking enzymes (e.g., CLEC). Immobilization of an enzyme generally refers to covalent attachment of the enzyme to a material's surface at the molecular level or scale, to limit conformational changes in the presence of solvents that result in loss of activity, prevent enzyme aggregation, improve enzyme resistance to proteolytic digestion by limiting conformational changes and exposure of cleavage sites, and to increase the surface area of an exposed enzyme to a substrate for catalytic activity [In “Engineering of/with Lipases” (F. Xavier Malcata., Ed.) pp. 457-458, 1996; “Methods in non-aqueous enzymology” (Gupta, M. N., Ed.) p. 37, 2000]. Immobilization of an enzyme may be used to improve stability against oxidation (e.g., autooxidation) solvent, solute, or shear force denaturation, or self digestion; prevent loss of enzymes by dissolving into water or other solvents and being washed away, and providing an increased concentration of enzyme in a local area for highest yield of products. Often other properties such a selectivity, pH and temperature optimums, Km, etc. may be altered by immobilization. Various types of substrates for enzyme immobilization include reverse micelles, zeolite, Celite Hyflo Supercel, anion exchange resin, Celite® (diatomaceous earth), polyurethane foam particles, macroporous polypropylene Accurel® EP 100, macroporous anionic resin beads, polypropylene membrane, acrylic membrane, nylon membrane, cellulose ester membrane, polyvinylidene difuoride membrane, filter paper, teflon membrane, reverse micelles, ceramic membrane, macroporous packing particulate, polyamide, cellulose hollow fibre, resin or carrier, polypropylene membrane pretreated with a blocked copolymer, immunoglobins via enzyme-linked immunosorbent assay, agarose, ion-exchange resin, sol-gel (In “Engineering of/with Lipases” (F. Xavier Malcata., Ed.) pp. 298, 408, 409, 414, 422, 447, 448, 451, 461, 494, 501, 516, 546, 549, 1996; U.S. Pat. No. 4,939,090; Lopez, M. et al., 1998; “Methods in non-aqueous enzymology” (Gupta, M. N., Ed.) pp. 41-51, 63-65, 2000]. For example, a lipase incorporated in sol-gel had 100-fold improved activity (Reetz, M. et al., 1995). Though many immobilized lipolytic enzymes are purified enzymes, immobilized whole cell lipase biocatalysts have been described [In “Engineering of/with Lipases” (F. Xavier Malcata., Ed.), p. 88, 1996]. In some cases, enzymes or cells are immobilized by entrapment into gels formed from alginate, a carragenan, or polyacrylamide (Karube, I. et al., 1985; Qureshi, N. et al., 1985; Umemura, I. et al., 1984; Fukui, S, and Tanaka, A. 1984; Mori, T. et al., 1972; Martinek, K. et al., 1977).

Methods of immobilization include, for example, absorption, ionic binding, covalent attachment, or cross-linking, entrapment into a gel, entrapment into a membrane compartment, or a combination thereof (Kurt Faber, “Biotransformations in Organic Chemistry, a Textbook, Third Edition.” pp. 345-356, 1997). Lysine amino moieties and aspartate and glutamate carboxyl moieties can be used to chemically bind an enzyme to a solid support. For example, nitrobenzenic acid derivates may be used to acylate the active side lysine of phospholipase A2 to improve activity, and immobilize the enzyme to Reacti-Gel [see for example, in “Methods and Molecular Biology, Volume 109 Lipase and Phospholipase Protocols.” (Mark Doolittle and Karen Reue, Eds.), pp. 303-307, 1999]. Immobilization of epoxy-activated Candida rugosa lipase produces monoalkylation of lysine moieties that improves enzyme stability by enhancing resistance to other chemical reactions, and modifies substrate selectivity (Kurt Faber, “Biotransformations in Organic Chemistry, a Textbook, Third Edition.” Springer-verlag Berlin Heidelberg, p. 313, 1997; Beger, B. and Faber, 1991).

Absorption may be used to attach an enzyme onto a material where it is held by non-covalent (e.g., hydrogen bonding, Van der Waals forces) interactions. Examples of materials that may be used for absorption of an enzyme include a woodchip, an activated charcoal, an aluminum oxide, a diatomaceous earth (e.g., Celite), a cellulose material, a controlled pore glass, a siliconized glass bead, or a combination thereof. In some cases, the buffering capacity of an immobilization carrier, such as diatomaceous earth (e.g., Celite), can improve the catalytic rate or selectivity of a lipolytic enzyme (e.g., Pseudomonas sp. lipase), as acids produced by ester hydrolysis can alter local pH to detrimentally effect the reaction (Kurt Faber, “Biotransformations in Organic Chemistry, a Textbook, Third Edition.”, p. 114-115, 1997; “Lipases” (Borgstrom, B. and Brockman, H. L., Eds), p. 196, 1984].

Ion exchange resins, such as cation (e.g., carboxymethyl cellulose, Amberlite IRA), anion (e.g., sephadex, diethyl-aminoethylcellulose), or a combination thereof, may be used to immobilize an enzyme. Covalent bonding immobilization generally involves chemical reactions on an amino acid residue at an amino moiety (e.g., lysine's epsilon amino group), a phenolic moiety, a suflhydryl moiety, a hydroxyl moiety, a carboxy moiety, or a combination thereof, usually with a spacer chemical that is used to bind to the enzyme to a carrier. Examples of carriers that may be used to immobilize an enzyme by covalent bonds include porous glass via a spacer (e.g., an aminoalkylethoxy-chlorosilane, an aminoalkyl-chlorosilane); a polysaccharide polymer carrier (e.g., agarose, chitin, cellulose, dextran, starch) via reaction cyanogens bromide reactions; a synthetic co-polymer (e.g., polyvinyl acetate) via epichlorohydrin activation reactions; an epoxy-activate resin; a cation exchange resin activated to covalently bond by acid chloride conversion of carboxylic acids, or a combination thereof. Cross-linking enzymes may be interconnect an enzyme to a like or different enzyme, sometimes with a filler protein (e.g., an albumin) separating the enzyme molecules, via a biofunctional agent (e.g., a glutardialdehyde, dimethyl adipimidate, dimethyl suberimidate and hexamethylenediisocyanate). Gel entrapment includes incorporation of enzymes or cells into gel matrices (e.g., an alginate, a carragenan gel, a polyacrylamide gel, or a combination thereof) that can be formed into various shapes (Karube, I. et al., 1985; Qureshi, N. et al., 1985; Umemura, I. et al., 1984; Fukui, S. and Tanaka, A. 1984; Mori, T. et al., 1972; Martinek, K. et al., 1977; Kurt Faber, “Biotransformations in Organic Chemistry, a Textbook, Third Edition.” pp. 350-352, 1997). Membrane entrapment refers to restricting the space an enzyme functions in by being placed in a compartment, often imitating the separation of an enzyme that occurs inside a living cell (e.g., localization of an enzyme inside an organelle). Examples of membrane entrapment compositions include a micelle, a reversed micelle, a vesicle (e.g., a liposome), a synthetic membrane (e.g., a polyamide, a polyethersulfone) with a pore size smaller than the enzyme sequestering an enzyme (e.g., a membrane enclosed enzymatic catalysis or “MEEC”). However, a MEEC may reduce the function of many lipolytic enzymes, possibly due to interference with the interfacial activation process by this type of environment (Kurt Faber, “Biotransformations in Organic Chemistry, a Textbook, Third Edition.” pp. 345-356, 1997).

### I. Coatings

In some embodiments, a coating or surface treatement comprises a biomolecular composition. Coating and other surface treatments, and antimicrobial peptide compositions, and their preparation which may be used in light of the present disclosures have been described in U.S. patent application Ser. Nos. 10/655,345, 10/792,516, and 10/884,355, each incorporated by reference). A coating (“coat,” “surface coat,” “surface coating”) is “a liquid, liquefiable or mastic composition that is converted to a solid protective, decorative, or functional adherent film after application as a thin layer” (“Paint and Coating Testing Manual, Fourteenth Edition of the Gardner-Sward Handbook” (Koleske, J. V. Ed.), p. 696, 1995; and in “ASTM Book of Standards, Volume 06.01, Paint—Tests for Chemical, Physical, and Optical Properties; Appearance,” D16-00, 2002). Additionally, a thin layer is 5 um to 1500 um thick, including all intermediate ranges and combinations thereof. However, in many embodiments, it is contemplated that a coating will form a thin layer 15 um to 150 um thick, including all intermediate ranges and combinations thereof. Examples of a coating include a clear coating or a paint.

A surface is the outer layer of any solid object. The term “substrate,” in the context of a coating, is synonymous with the term “surface.” However, as “substrate” has a different meaning to those of skill in arts of enzymology and coatings, the term “surface” will be preferentially used herein for clarity. A surface wherein a coating has been applied, whether or not film formation has occurred, is known herein as a “coated surface.”

A coating generally comprises one or more materials that contribute to the properties of the coating, the ability of a coating to be applied to a surface, the ability of the coating to undergo film formation, and/or the properties of the produced film. Examples of such coating components include a binder, a liquid component, a colorizing agent, an additive, or a combination thereof, and such materials are contemplated for used in a coating. A coating typically comprises a material often referred to as a “binder,” which is the primary material in a coating capable of film formation. Often the binder is the coating component that dominates conferring a physical and/or chemical property to a coating and/or film. Examples of properties of a binder typically affects include chemical reactivity, minimum film formation temperature, minimum T_{g}, volume fraction solids, a rheological property (e.g., viscosity), film moisture resistance, film UV resistance, film heat resistance, film weathering resistance, adherence, film hardness, film flexibility, or a combination thereof. Consequently, different categories of coatings may be identified herein by the binder used in the coating. For example, a binder may be an oil, a chlorinated rubber, or an acrylic, and examples of a coating comprising such binders include an oil coating, a chlorinated rubber-topcoat, an acrylic-lacquer, etc. In certain embodiments, a biomolecular composition may function as a binder, particularly in aspects wherein the coating comprises another thermosetting binder that may crosslink to the chemical moieties (e.g., hydroxyl moieties, amine moieties, polyols, carboxyl moieties, fatty acids, double bonds, etc.) typically found in cells.

In many embodiments, a coating will comprise a liquid component (e.g., a solvent, a diluent, a thinner), which often confers and/or alters the coating's rheological properties (e.g., viscosity) to ease the application of the coating to a surface. In some embodiments, a coating will comprise a colorizing agent (e.g., a pigment), which usually functions to alter an optical property of a coating and/or film. In certain embodiments, a biomolecular composition is a colorizing agent. In particular embodiments, a colorizing agent comprising a biomolecular composition is an extender, a pigment, or a combination thereof. In other embodiments, a coating comprises a colorizing agent that comprises a biomolecular composition. A coating will often comprise an additive which is a composition incorporated into a coating to reduce and/or prevent the development of a physical, chemical, and/or aesthetic defect in the coating and/or film; confer some additional desired property to a coating and/or film; or a combination thereof. Examples of an additive include an accelerator, an adhesion promoter, an antioxidant, an antiskinning agent, a coalescing agent, a defoamer, a dispersant, a drier, an emulsifier, a fire retardant, a flow control agent, a gloss aid, a leveling agent, a marproofing agent, a slip agent, a thickener, a UV stabilizer, a viscosity control agent, a wetting agent, or a combination thereof. In certain embodiments, a biomolecular composition comprises an additive. In particular embodiments, an additive comprising a biomolecular composition comprises a viscosity control agent, a dispersant, or a combination thereof. In other embodiments, a coating comprises an additive that comprises a biomolecular composition. A contaminant is a material that is unintentionally added to a coating, and may be volatile and/or non-volatile component of a coating and/or film. A coating component may be categorized as possessing more than one defining characteristic, and thereby simultaneously functioning in a coating composition as a combination of a binder, a liquid component, a colorizing agent, and/or additive. Different coating compositions are described herein as examples of coatings with varying sets of properties.

In certain embodiments, a coating may be stored in a container (“pot”) prior to application. In certain aspects, the coating is a multi-pack coating which is a coating wherein different components are stored in a plurality of containers (e.g., a kit). Typically, this is done to reduce film formation during storage for certain types of coatings. The components are admixed prior to and/or during application. However, in certain embodiments, it is specifically contemplated that a coating comprising a biomolecular composition is a multi-pack coating. In specific aspects, the coating is a two-pack coating, three-pack coating, four-pack coating, five-pack coating, or more wherein the coating components are stored in separate containers. In certain aspects, 0.000001% to 100%, including all intermediate ranges and combinations thereof, of the biomolecular composition is stored in a separate container from a coating component. It is contemplated that separate storage may reduce undesirable microoganism growth in the coating component, damage to the biomolecular composition by the coating component, increase the storage life (“pot life”) of a coating, reduce the amount of a preservative in a coating, or a combination thereof. In certain facets, it is contemplated that the coating components of a container holding the biomolecular composition materialmay further include a coating component such as a preservative, a wetting agent, a dispersing agent, a liquid component, a rheological modifier, or a combination thereof. It is contemplated that a preservative may reduce undesirable growth of a microoganism, whether the microoganism is derived from the biomolecular composition or a contaminating microorganism. It is contemplated that a wetting agent, a dispersing agent, a liquid component, a rheological modifier, or a combination thereof, may promote ease of admixing of coating components in a multi-pack coating. In certain aspects, a three-pack coating or four-pack coating may be used, wherein the first container and the second container contain coating components separated to reduced film formation during storage, and a third container comprises 0.001% to 100%, including all intermediate ranges and combinations thereof, of the biomolecular composition. In certain facets, a multi-pack coating may be used to separate two or more preparations of the biomolecular composition.

A coating may be applied to a surface using any technique known in the art. A in the context of a coating, “application,” “apply,” or “applying” is the process of transferring of a coating to a surface to produce a layer of coating upon the surface. As known herein, an “applicator” is a devise that is used to apply the coating to a surface. Examples of an applicator include a brush, a roller, a pad, a rag, a spray applicator, etc. Application techniques that are contemplated as suitable for a user of little or no particular skill include, for example, dipping, pouring, siphoning, brushing, rolling, padding, ragging, spraying, etc. Certain types of coatings may be applied using techniques contemplated as more suitable for a skilled artisan such as anodizing, electroplating, and/or laminating of a polymer film onto a surface.

In certain embodiments, the layer of coating undergoes film formation (“curing,” “cure”), which is the physical and/or chemical change of a coating to a solid when in the form of a layer upon the surface. In certain aspects, a coating may be prepared, applied and cured at an ambient condition, a baking condition, or a combination thereof. An ambient condition is a temperature range between −10° C. to 40° C., including all intermediate ranges and combinations thereof. As used herein, a “baking condition” or “baking” is contacting a coating with a temperature above 40° C. and/or raising the temperature of a coating above 40° C., typically to promote film formation. Examples of baking the coating include contacting a coating and/or raising the temperature of coating to 40° C. to 300° C., or more, including all intermediate ranges and combinations thereof. Various coatings may be applied and/or cured at ambient conditions, baking conditions, or a combination thereof.

It is contemplated that in general embodiments, a coating comprising a biomolecular composition may be prepared, applied and cured at any temperature range described herein or would be applicable in the art in light of the present disclosures. An example of such a temperature range is −100° C. to 300° C., or more, including all intermediate ranges and combinations thereof. However, a biomolecular composition material may further comprise a desired biomolecule (e.g., a colorant, an enzyme), whether endogenously or recombinantly produced, that may have a reduced tolerance to temperature. It is contemplated that the temperature that can be tolerated by a biomolecule will vary depending on the specific biomolecule used in a coating, and will generally be within the range of temperatures tolerated by the living organism from which the biomolecule was derived. For example, a coating comprising a biomolecular composition, wherein the biomolecular composition comprises an enzyme, that the coating is prepared, applied and cured at −100° C. to 110° C., including all intermediate ranges and combinations thereof. For example, it is contemplated that a temperature of −100° C. to 40° C. including all intermediate ranges and combinations thereof, will be suitable for many enzymes (e.g., a wild-type sequence and/or a functional equivalent) derived from an eukaryote, while temperatures up to, for example −100° C. to 50° C. including all intermediate ranges and combinations thereof, may be tolerated by enzymes derived from many prokaryotes.

The type of film formation that a coating may undergo depends upon the coating components. A coating may comprise, for example, volatile coating components, non-volatile coating components, or a combination thereof. In certain aspects, the physical process of film formation comprises loss of 1% to 100%, including all intermediate ranges and combinations thereof, of a volatile coating component. In general embodiments, a volatile component is lost by evaporation. In certain aspects, loss of a volatile coating component during film formation reaction is promoted by baking the coating. Examples of volatile coating components include a coalescing agent, a solvent, a thinner, a diluent, or a combination thereof. A non-volatile component of the coating remains upon the surface. In specific aspects, the non-volatile component forms a film. Examples of non-volatile coating components include a binder, a colorizing agent, a plasticizer, a coating additive, or a combination thereof. It is contemplated that a cell-based particulate material will be a non-volatile coating component. In specific aspects, a coating component may undergo a chemical change to form a film. In general embodiments, a binder undergoes a cross-linking (e.g., polymerization) reaction to produce a film. In general embodiments, a chemical film formation reaction occurs spontaneously under ambient conditions. In other aspects, a chemical film formation reaction is promoted by irradiating the coating, heating the coat, or a combination thereof. In some embodiments, irradiating the coating comprises exposing the coating to electromagnetic radiation, particle radiation, or a combination thereof. Examples of electromagnetic radiation used to irradiate a coating include UV radiation, infrared radiation, or a combination thereof. Examples of particle radiation used to irradiate a coating include electron-beam radiation. Often irradiating the coating induces an oxidative and/or free radical chemical reaction that cross-links of one or more coating components.

However, in some alternate embodiments, it is contemplated that a coating undergoes a reduced amount of film formation than such a solid film is not produced, or does not undergo film formation to a measurable extent during the period of time it is used on a surface. Such a coating is referred to herein as a “non-film forming coating.” Such a non-film forming coating may be prepared, for example, by increasing the non-volatile component in a thermoplastic coating (e.g., increasing plasticizer content in a liquid component), reducing the amount of a coating component that contributes to the film formation chemical reaction (e.g., a binder, a catalyst), increasing the concentration of a component that inhibits film formation (e.g., an antioxidant/radical scavenger in an oxidation/radical cured thermosetting coating), reducing the contact with an external a curing agent (e.g., radiation, baking), selection of a non-film formation binder produced from components that lack crosslinking moieties, selection of a non-film formation binder that lack sufficient size to undergo thermoplastic film formation, or a combination thereof. As used herein, a “non-film formation binder” refers to a molecule that is chemically similar to a binder, but lacks sufficient size and/or crosslinking moiety to undergo film formation. For example, a coating may be prepared by selection of an oil-based binder that lacks sufficient double bonds to undergo sufficient crosslinking reactions to produce a film. In another example, a non-film formation binder may be selected that lacks sufficient crosslinking moieties such as an epoxide, an isocyanate, a hydroxyl, a carboxyl, an amine, an amide, a silicon moiety, etc., to produce a film by thermosetting. Such a non-film formation binder may be prepared by chemical modification of a binder, such as, for example, a crosslinking reaction with a small molecule (e.g., less than 1 kDa) that comprises a moiety capable of reaction with a binder's crosslinking moiety, to produce a chemically blocked binder moiety that is inert to a further crosslinking reaction. In another example, a thermoplastic binder typically comprises a molecule 29 kDa to 1000 kDa or more in size, though more specific, ranges for different binders (e.g., acrylics, polyvinyls, etc.) are described herein. Film formation may be reduced or prevented by selection of a like molecule that is too small to effectively undergo thermoplastic film formation. An example would be selection of a non-film formation binder molecule between 1 kDa to 29 kDa in molecular weight, including all intermediate ranges and combinations thereof.

In other alternative embodiments, a coating may undergo film formation, but produce a film whose properties makes it more suited for a temporary use. Such a temporary film will generally possess a poor and/or low rating for a property that would confer longevity in use. For example, a film with a poor abrasion (e.g., scrub) resistance, a poor solvent resistance, a poor water resistance, a poor weathering property (e.g., UV resistance), a poor adhesion property, a poor microorganism/biological resistance, or a combination thereof, may be selected as a temporary film. Such a “poor” or “low” property would be determined by standards in the art, and often the detection of the coating property (e.g., a change in the coating's color, gloss, loss of coating material) and/or is a rating in the half of a standard test rating scale and/or a detectable that is associated with a reduced longevity of use. In one aspect, a film may have poor adhesion for a surface, allowing ease of removal by stripping and/or peeling. In certain aspects, a poor or low adhesion rating on a scale of 0 (lowest adhesion) to 5 is denoted 2A, 1A, 0A, 2B, 1B, 0B, including all intermediate ranges and combinations thereof, as described in “ASTM Book of Standards, Volume 06.01, Paint—Tests for Chemical, Physical, and Optical Properties; Appearance,” D3359-97, 2002. Other examples of standard adhesion assays that may be used to determine a poor or low adhesion property rating include “ASTM Book of Standards, Volume 06.01, Paint—Tests for Chemical, Physical, and Optical Properties; Appearance,” D5179-98 and D2197-98, 2002; “ASTM Book of Standards, Volume 06.02, Paint—Products and Applications; Protective Coatings; Pipeline Coatings,” D4541-02, D3730-98, D4145-83, D4146-96, and D6677-01, 2002; and “ASTM Book of Standards, Volume 06.02, Paint—Products and Applications; Protective Coatings; Pipeline Coatings,” D5064-01, 2002. In other aspects, a poor or low abrasion rating for a coating is denoted as a detectable gloss, color and/or material erosion, such as an increase (“I”), large increase (“LI”), decrease (“D”), or large decrease (“LD”) gloss change, a slightly darker (“SD”), considerably darker (“CD”), slightly lighter (“SL”) or considerably lighter (“CL”) color change, a slight (“S”) or moderate (“M”) erosion change, including all intermediate ranges and combinations thereof for gloss, color and/or erosion, as described in “ASTM Book of Standards, and Volume 06.02, Paint—Products and Applications; Protective Coatings; Pipeline Coatings,” D4828-94, 2002. Additional examples of standard abrasion tests that may be used to determine a poor or low abrasion resistance property rating include those described in “ASTM Book of Standards, Volume 06.01, Paint—Tests for Chemical, Physical, and Optical Properties; Appearance,” D968-93 and D4060-01, 2002; and “ASTM Book of Standards, and Volume 06.02, Paint—Products and Applications; Protective Coatings; Pipeline Coatings,” D3170-01, D4213-96, D2486-00, D3450-00, D6736-01, and D6279-99e1, 2002. Weathering resistance is described in “ASTM Book of Standards, Volume 06.01, Paint—Tests for Chemical, Physical, and Optical Properties; Appearance,” D4141-01, D1729-96, D660-93, D661-93, D662-93, D772-86, D4214-98, D3274-95, D714-02, D1654-92, D2244-02, D523-89, D1006-01, D1014-95, and D1186-01, 2002; “ASTM Book of Standards, Volume 06.02, Paint—Products and Applications; Protective Coatings; Pipeline Coatings,” D3719-00, D610-01, D1641-97, D2830-96, and D6763-02, 2002; and “ASTM Book of Standards, Volume 06.01, Paint—Tests for Chemical, Physical, and Optical Properties; Appearance,” D822-01, D4587-01, D5031-01, D6631-01, D6695-01, D5894-96, and D4141-01, 2002; “ASTM Book of Standards, Volume 06.02, Paint—Products and Applications; Protective Coatings; Pipeline Coatings,” D5722-95, D3361-01 and D3424-01, 2002. Examples of poor weathering resistance includes a blistering rating of dense (“D”), medium dense (“MD”), medium (“M”) blistering, a failure at scribe, which is a measure of corrosion and paint loss at the site of contact with a tool known as a scribe, in the range of 0 to 5, a rating of the unscribed areas of 0 to 5, a rust grade rating of a coated steel surface of 0 to 5, a general appearance rating of 0 to 5, a cracking rating of 0 to 5, a checking rating of 0 to 5, a dulling rating of 0 to 5, and/or a discoloration rating of 0 to 5, including all intermediate ranges and combinations thereof, respectively, as described in “ASTM Book of Standards, Volume 06.01, Paint—Tests for Chemical, Physical, and Optical Properties; Appearance,” D714-02 and D1654-92, 2002; and “ASTM Book of Standards, Volume 06.02, Paint—Products and Applications; Protective Coatings; Pipeline Coatings,” D610-01 and D1641-97, 2002. In additional aspects, a poor or low solvent resistance rating for a coating is denoted as a solvent resistance rating of 0 to 2, a coating removal efficiency rating of 3 to 5, an effect of coating removal on the condition of the surface of 0 to 2, including all intermediate ranges and combinations thereof, respectively, as described in “ASTM Book of Standards, Volume 06.02, Paint—Products and Applications; Protective Coatings; Pipeline Coatings,” D4752-98, 2002; and “ASTM Book of Standards, Volume 06.02, Paint—Products and Applications; Protective Coatings; Pipeline Coatings,” D6189-97, 2002. An additional example of a standard solvent resistance assay is described in “ASTM Book of Standards, Volume 06.02, Paint—Products and Applications; Protective Coatings; Pipeline Coatings,” D5402-93, 2002. In further aspects, a poor or low water resistance rating for a coating is denoted as a discernable change in a coating's color, blistering, adhesion, softening, and/or embrittlement upon conducting an assay as described in “ASTM Book of Standards, Volume 06.01, Paint—Tests for Chemical, Physical, and Optical Properties; Appearance,” D2247-02 and D4585-99, 2002. Further assays for water resistance are described in “ASTM Book of Standards, Volume 06.01, Paint—Tests for Chemical, Physical, and Optical Properties; Appearance,” D870-02, D1653-93, D1735-02, 2002; and “ASTM Book of Standards, Volume 06.02, Paint—Products and Applications; Protective Coatings; Pipeline Coatings,” D2065-96, D2921-98, D3459-98, and D6665-01, 2002.

In particular aspects, growth of cells, particularly microorganisms, may produce a coating or film with reduced stability, film formation capability, durability, etc. Such a non-film formatting film and/or temporary film may be prepared by the inclusion of the cell-based particulate material, particularly in embodiments wherein the cell-based particulate material is not a sterilized cell-based particulate material, the coating has a reduced concentration of biocide (e.g., 0% to 99.9999%, including all intermediate ranges and combinations thereof, a typically used concentration for a coating comprising the cell-based particulate material), the coating comprises a nutrient (e.g., a cell-based particulate material, other digestible material, vitamins, trace minerals, etc.) as a coating component (e.g., an additive) that promotes cell growth, or a combination thereof.

In additional aspects, a poor or low microorganism/biological resistance rating for a coating is denoted as a colony recovery/growth rating of 2 to 4, a discoloration/disfigurement rating of 0 to 5, a fouling resistance (“F.R.”) or antifouling film (“A.F”) rating of 0 to 70, and observed growth (e.g., fungal growth) on specimens of 2 to 4, including all intermediate ranges and combinations thereof, respectively, as described in “ASTM Book of Standards, Volume 06.01, Paint—Tests for Chemical, Physical, and Optical Properties; Appearance,” D3274-95, D2574-00, D3273-00, D5589-97 and D5590-00, 2002; and in “ASTM Book of Standards, Volume 06.02, Paint—Products and Applications; Protective Coatings; Pipeline Coatings,” D3623-78a, 2002. An additional example of a standard microorganism/biological resistance assay is described in “ASTM Book of Standards, Volume 06.01, Paint—Tests for Chemical, Physical, and Optical Properties; Appearance,” D4610-98 and D3456-86, 2002; in “ASTM Book of Standards, Volume 06.02, Paint—Products and Applications; Protective Coatings; Pipeline Coatings,” D4938-89, D4939-89, D5108-90, D5479-94, D6442-99, D6632-01, D4940-98 and D5618-94, 2002; and “ASTM Book of Standards, Volume 06.03, Paint—Pigments, Drying Oils, Polymers, Resins, Naval Stores, Cellulosic Esters, and Ink Vehicles,” D912-81 and D964-65, 2002.

In another example, a film may have a poor resistance to an environmental factor, and subsequently fail (e.g., crack, peel, chalk, etc.) to remain a viable film upon the surface. For example, a film that undergoes chalking is specifically contemplated. Chalking is the erosion a coating, typically by degradation of the binder due to various environmental forces (e.g., UV irradiation). It is contemplated that in some embodiments, chalking may be desirable, to remove a contaminant from the surface of a film and/or expose a component of the film (e.g., a biomolecular composition) to the surface of the coating. In some aspects, a chalking coating has a chalking rating on a “Wet Finger Method” of visible or severe and/or a chalk reflectance rating of 0 to 5, including all intermediate ranges and combinations thereof, as described in “ASTM Book of Standards, Volume 06.01, Paint—Tests for Chemical, Physical, and Optical Properties; Appearance,” D4214-98, 2002. A self-cleaning coating is a film with a desirable high chalking property. It is further contemplated that in many aspects the layer of non-film forming coating, a temporary film and/or a self-cleaning film may be removed from a surface with ease. In such embodiments, a non-film forming coating, a temporary film, a self-cleaning film, or a combination thereof would be more suitable for a temporary use upon a surface, due to the ability to be applied as a layer and easily removed when its presence is no longer desired. In these embodiments, it is contemplated that the non-film forming coating, the temporary film, the self-cleaning film, or a combination thereof, is desired for a use upon a surface that lasts a temporary period of time, such as, for example, 1 to 60 seconds, 1 to 24 hours, 1 to 7 days, 1 to 10 weeks, 1 to 6 months, including all intermediate ranges and combinations thereof, respectively.

In some embodiments, a plurality of coating layers, known herein as a “multicoat system” (“multicoating system”), may be applied upon a surface. The coating selected for use in a specific layer may differ from an additional layer of the multicoat system. This selection of coatings with differing components and/or properties is typically done to sequentially confer, in a desired pattern, the properties of differing coatings to a coated surface and/or multicoat system. Examples of a coating that may be selected for use, either alone or in a multicoat system, include a sealer, a water repellent, a primer, an undercoat, a topcoat, or a combination thereof. A sealer is coating applied to a surface to reduce or prevent absorption by the surface of a subsequent coating layer and/or a coating component thereof, and/or to prevent damage to the subsequent coating layer by the surface. A water repellant is a coating applied to a surface to repel water. A primer is a coating that is applied to increase adhesion between the surface and a subsequent layer. In typical embodiments a primer-coating, a sealer-coating, a water repellent-coating, or a combination thereof is applied to porous surface. Examples of a porous surface include drywall, wood, plaster, masonry, damaged and/or degraded film, corroded metal, or a combination thereof. In certain aspects, the porous surface is not coated or lacks a film prior to application of a primer, sealer, water repellent, or combination thereof. An undercoat is a coating applied to a surface to provide a smooth surface for a subsequent coat. A topcoat (“finish”) is a coating applied to a surface for a protective and/or decorative purpose. Of course, a sealer, water repellent, primer, undercoat, and/or topcoat may possess additional protective, decorative, and/or functional properties. Additionally, the surface a sealer, water repellent, primer, undercoat, and/or topcoat are applied to may be a coated surface such as a coating and/or film of a layer of a multicoat system. In certain embodiments, a multicoat system may comprise any combination of a sealer, water repellent, primer, undercoat, and/or topcoat. For example, a multicoat system may comprise any of the following combinations: a sealer, a primer and a topcoat; a primer and topcoat; a water repellent, a primer, undercoat, and topcoat; an undercoat and topcoat; a sealer, an undercoat, and a topcoat; a sealer and topcoat; a water repellent and topcoat, etc. In particular aspects, a coating layer may comprise properties that would be a combination of those associated with different coating types such as a sealer, water repellent, primer, undercoat, and/or topcoat. In such instances, such a combination coating and/or film is designated by a backslash “/” separating the individual coating designations encompassed by the layer. Examples of such a coating layer comprising a plurality of functions include a sealer/primer coating, a sealer/primer/undercoat coating, a sealer/undercoat coating, a primer/undercoat coating, a water repellant/primer coating, an undercoat/topcoat coating, a primer/topcoat coating, a primer/undercoat/topcoat coating, etc. In embodiments wherein the coated surface comprises a particular type of coating, then the coated surface may be known herein by the type of coating such as a “painted surface,” a “clear coated surface,” a “lacquered surface,” a “varnished surface,” a “water repellant/primered surface,” an “primer/undercoat-topcoated surface,” etc.

In specific aspects, a multicoat system may comprise a plurality of layers of the same type, such as, for example, 1 to 10 layers, including all intermediate ranges and combinations thereof, of a sealer, water repellent, primer, undercoat, topcoat, or any combination thereof. In specific facets, a multicoat system comprises a plurality of layers of the same coating type, such as, for example, 1 to 10 layers, including all intermediate ranges and combinations thereof, of a sealer, water repellent, primer, undercoat, or topcoat. In embodiment where a coating does not comprise a multicoat system, but a single layer of coating applied to a surface, such a layer, regardless of typical function in a multicoat system, is regarded herein as a topcoat.

1. Paints

A paint is a “pigmented liquid, liquefiable or mastic composition designed for application to a substrate in a thin layer which is converted to an opaque solid film after application. Used for protection, decoration or identification, or to serve some functional purpose such as the filling or concealing of surface irregularities, the modification of light and heat radiation characteristics, etc.” [“Paint and Coating Testing Manual, Fourteenth Edition of the Gardner-Sward Handbook” (Koleske, J. V. Ed.), p. 696, 1995]. However, as certain coatings disclosed herein are non-film forming coatings, this definition is modified herein to encompass a coating with the same properties of a film forming paint, with the exception that it does not produce a solid film. In particular embodiments, a non-film forming paint possesses a hiding power sufficient to concealing surface feature comparable to an opaque film.

Hiding power is the ability of a coating and/or film to prevent light from being reflected from a surface, particularly to convey the suface's visual pattern. Opacity is the hiding power of a film. An example of hiding power would be the ability of a paint-coating to visually block the appearance of grain and color of a wooden surface, as opposed to a clear varnish-coating allowing the relatively unobstructed appearance of wood to pass through the coating. Standard techniques for determining the hiding power of a coating and/or film (e.g., paint, a powder coating) are described, for example, in “ASTM Book of Standards, Volume 06.01, Paint—Tests for Chemical, Physical, and Optical Properties; Appearance,” E284-02b, D344-97, D2805-96a, D2745-00 and D6762-02a 2002; “ASTM Book of Standards, Volume 06.02, Paint—Products and Applications; Protective Coatings; Pipeline Coatings,” D5007-99, D5150-92 and D6441-99, 2002; and “Paint and Coating Testing Manual, Fourteenth Edition of the Gardner-Sward Handbook” (Koleske, J. V. Ed.), pp. 481-506, 1995.

2. Clear-Coatings

A clear-coating is a coating that is not opaque and/or does not produce an opaque solid film after application. A clear-coating and/or film may be transparent or semi-transparent (e.g., translucent). A clear-coating may be colored or non-colored. In certain embodiments, reducing the content of a pigment in a paint composition may produce a clear-coating. Additionally, a clear-coating may comprise a lacquer, a varnish, a shellac, a stain, a water repellent coating, or a combination thereof. Though some opaque coatings are referred to in the art as a lacquer, a varnish, a shellac, or a water repellent coating, all such opaque coatings are considered as paints herein (e.g., a lacquer-paint, a varnish-paint, a shellac-paint, a water repellent paint).

a. Varnishes

A varnish is a thermosetting coating that converts to a transparent or translucent solid film after application. In general embodiments, a varnish is a wood-coating. A varnish comprises an oil and a dissolved binder. In general embodiments, the oil comprises a drying oil, wherein the drying oil functions as an additional binder. In other embodiments, the binder is solid at ambient conditions prior to dissolving into the oil and/or an additional liquid component of the varnish. Examples of a dissolvable binder include resins obtained from a natural source (e.g., a Congo resin, a copal resin, a damar resin, a kauri resin), a synthetic resin, or a combination thereof. In specific aspects, the additional liquid component comprises a solvent such as a hydrocarbon solvent. In some facets, the solvent is added to reduce viscosity of the varnish. A varnish may further comprise a coloring agent, including a pigment, for such purposes as conferring or altering a color, gloss, sheen, or a combination thereof. A varnish undergoes thermosetting film formation by oxidative cross-linking. In certain aspects, a varnish may additionally undergo film-formation by evaporation of a volatile component. The dissolved binder generally functions to shorten the time to film-formation relative to certain measures (e.g., dryness, hardness), though the final cross-linking reaction time may not be significantly or measurably shortened. Standards for determining a varnish-coating and/or film's properties are described in, for example, “ASTM Book of Standards, Volume 06.03, Paint—Pigments, Drying Oils, Polymers, Resins, Naval Stores, Cellulosic Esters, and Ink Vehicles,” D154-85, 2002.

b. Lacquers

A lacquer is a thermoplastic, solvent-borne coating that converts to a transparent or translucent solid film after application. In general embodiments, a lacquer is a wood-coating. A lacquer-coating comprises a thermoplastic binder dissolved in a liquid component comprising an active solvent. Examples of a thermoplastic binder include a cellulosic binder (e.g., nitrocellulose, cellulose acetate), a synthetic resin (e.g., an acrylic), or a combination thereof. In certain aspects, a liquid component comprises an active solvent, a latent solvent, diluent, a thinner, or a combination thereof. In certain embodiments, a lacquer is nonaqueous dispersion (“NAD”) lacquer, wherein the content of solvent is not sufficient to fully dissolve the thermoplastic binder. In certain aspects, a lacquer may comprise an additional binder (e.g., an alkyd), a colorant, a plasticizer, or a combination thereof. Film formation of a lacquer occurs by loss of the volatile components, typically through evaporation.

Standards for a lacquer-coating and/or film's composition (e.g., a lacquer, a pigmented-lacquer, a nitrocellulose lacquer, a nitrocellulose-alkyd lacquer), physical and/or chemical properties (e.g., heat and cold resistance, hardness, film-formation time, stain resistance, particulate material dispersion), and procedures for testing a lacquer's composition/properties, are described in, for example, in “ASTM Book of Standards, Volume 06.02, Paint—Products and Applications; Protective Coatings; Pipeline Coatings,” D333-01, D2337-01, D3133-01, D365-01, D2091-96, D2198-02, D2199-82, D2571-95 and D2338-02, 2002.

c. Shellacs

A shellac is similar to a lacquer, but the binder does not comprise a nitrocellulose binder, and the binder is soluble in alcohol, and the binder is obtained from a natural source. In some embodiments, a binder comprises Laciffer lacca beetle secretion. In general embodiments, a shellac comprises a liquid component (e.g., alcohol). In specific aspects, the additional liquid component comprises a solvent. In some facets, the liquid component is added to reduce viscosity of the varnish. In other embodiments, a shellac undergoes rapid film formation. Standards for a shellac-coating and/or film's composition and properties are described in, for example, “ASTM Book of Standards, Volume 06.03, Paint—Pigments, Drying Oils, Polymers, Resins, Naval Stores, Cellulosic Esters, and Ink Vehicles,” D29-98 and D360-89, 2002.

d. Stains

A stain is a clear or semitransparent coating formulated to change the color of surface. In general embodiments, a stain is a wood-coating designed to color or protect a wood surface but not conceal the grain pattern or texture. A stain comprises a binder such as an oil, an alkyd, or a combination thereof. Often a stain comprises a low solid content. A low solids content for a wood stain is less than 20% volume of solids. The low solid content of a stain promotes the ability of the coating to penetrate the material of the wooden surface. This property is often used to, for example, to promote the incorporation of a fungicide that may be comprised within the stain into the wood. In certain alternative aspects, a stain comprises a high solids content stain, wherein the solid content is 20% or greater, may be used on a surface to produce a film possessing the property of little or no flaking. In other alternative aspects, a water-borne stain may be used such as a stain comprising a water-borne alkyd. A stain typically further comprises a liquid component (e.g., a solvent), a fungicide, a pigment, or a combination thereof. In other aspects, a stain comprises a water repellent hydrophobic compound so it functions as a water repellent-coating (“stain/water repellent-coating”). Examples of a water repellent hydrophobic compound a stain may comprise include a silicone oil, a wax, or a combination thereof. Examples of a fungicide include a copper soap, a zinc soap, or a combination thereof. Examples of a pigment include a pigment that is similar in color to wood. Examples of such pigments include a red pigment (e.g., a red iron oxide) a yellow pigment (e.g., a yellow iron oxide), or a combination thereof. Standards procedures for testing a stain's (e.g., an exterior stain) properties, are described in, for example, in “ASTM Book of Standards, Volume 06.02, Paint—Products and Applications; Protective Coatings; Pipeline Coatings,” D6763-02, 2002.

e. Water Repellent-Coatings

A water repellent-coating is a coating that comprises hydrophobic compounds that repel water. A water repellent-coating is typically applied to a surface susceptible to water damage, such as metal, masonry, wood, or a combination thereof. A water repellent-coating typically comprises a hydrophobic compound and a liquid component. In specific embodiments, a water repellent-coating comprises 1% to 65% hydrophobic compound, including all intermediate ranges and combinations thereof. Examples of a hydrophobic compound that may be selected include an acrylic, a siliconate, a metal-searate, a silane, a siloxane, a parafinnic wax, or a combination thereof. A water repellent may be a water-borne coating, or a solvent-borne coating. A solvent-borne water repellent-coating typically comprises a solvent that dissolves the hydrophobic compound. Examples of solvents include an aliphatic, an aromatic, a chlorinated solvent, or a combination thereof.

In certain embodiments, a water repellent-coating, undergoes film formation, penetrates pores, or a combination thereof. In certain aspects, an acrylic-coating, a silicone-coating, or a combination thereof, undergoes film formation. In other aspects, a metal-searate, a silane, a siloxane, a parafinnic wax, or a combination thereof, penetrates pores in a surface. In some facets, a water repellent-coating (e.g., a silane, a siloxane) covalently bonds to a surface and/or pore (e.g., masonry). Standards for a water repellent-coating and/or film's composition and properties are described in, for example, “ASTM Book of Standards, Volume 06.02, Paint—Products and Applications; Protective Coatings; Pipeline Coatings,” D2921-98, 2002; and in “Paint and Coating Testing Manual, Fourteenth Edition of the Gardner-Sward Handbook,” (Koleske, J. V. Ed.), pp. 748-750, 1995. Alternatively, standards for a sealer-coating (e.g., a floor sealer) and/or film's composition and properties are described in, for example, “ASTM Book of Standards, Volume 06.02, Paint—Products and Applications; Protective Coatings; Pipeline Coatings,” D1546-96, 2002;

3. Coating Categories by Use

In light of the present disclosures, a coating may be prepared and applied to any surface. However, it is contemplated that the coating components and methods described herein are selected for a particular application to provide a coating and/or film with properties best suited for a particular use. For example, a coating used in an external environment could comprise a coating component of superior UV resistance than a coating used in interior environment. In another example, a film used upon a surface of a washing machine could comprise a component that confers superior moisture resistance than a component of a film for use upon a ceiling surface. In a further example, a coating applied to the surface of an assembly line manufactured product could comprise components suitable for application by a spray applicator. Various properties of coating components are described herein to provide guidance to the selection of specific coating compositions with a suitable set of properties for a particular use.

A coating may be classified by its end use, including, for example, as an architectural coating, an industrial coating, a specification coating, or a combination thereof. An architectural coating is “an organic coating intended for on-site application to interior or exterior surfaces of residential, commercial, institutional, or industrial buildings, in contrast to industrial coatings. They are protective and decorative finishes applied at ambient conditions” [“Paint and Coating Testing Manual, Fourteenth Edition of the Gardner-Sward Handbook” (Koleske, J. V. Ed.), p. 686, 1995)] An industrial coating is a coating applied in a factory setting, typically for a protective and/or aesthetic purpose. A specification coating (“specification finish coating”) is a coating formulated to a “precise statement of a set of requirements to be satisfied by a material, produce, system, or service that indicates the procedures for determining whether each of the requirements are satisfied” [“Paint and Coating Testing Manual, Fourteenth Edition of the Gardner-Sward Handbook” (Koleske, J. V. Ed.), p. 891, 1995]. Often, a coating may be categorized as a combination of an architectural coating, an industrial coating, and/or a specification coating. For example, a coating for the metal surfaces of ships may be classified as specification coating, as specific criteria of water resistance and corrosion resistance are required in the film, but typically such a coating can be classified as an industrial coating, since it would typically be applied in a factory. Various examples of an architectural coating, an industrial coating and/or a specification coating and coating components are described herein. Additionally, architectural coatings, industrial coatings, specification coatings examples are described, for example, in “Paint and Surface Coatings: Theory and Practice” 2^{nd} Edition, pp. 190-192, 1999; in “Paints, Coatings and Solvents” 2^{nd} Edition, pp. 330-410, 1998; in “Organic Coatings: Science and Technology, Volume 1: Film Formation, Components, and Appearance” 2 d Edition, pp. 138 and 317-318.

a. Architectural Coatings

An architectural coating (“trade sale coating,” “building coating,” “decorative coating,” “house coating”) is a coating suitable to coat surface materials commonly found as part of buildings and/or associated objects (e.g., furniture). Examples of a surface an architectural coating is typically applied to include, a plaster surface, a wood surface, a metal surface, a composite particle board surface, a plastic surface, a coated surface (e.g., a painted surface), a masonry surface, a floor, a wall, a ceiling, a roof, or a combination thereof. Additionally, an architectural coating may be applied to an interior surface, an exterior surface, or a combination thereof. An interior coating generally possesses properties such as minimal odor (e.g., no odor, very low VOC), good blocking resistance, print resistance, good washability (e.g., wet abrasion resistance), or a combination thereof. An exterior coating typically is selected to possess good weathering properties. Examples of coating type commonly used as an architectural coating include an acrylic-coating, an alkyd-coating, a vinyl-coating, a urethane-coating, or a combination thereof. In certain aspects, a urethane-coating is applied to a piece of furniture. In other facets, an epoxy-coating, a urethane-coating, or a combination thereof, is applied to a floor. In some embodiments, an architectural coating is a multicoat system. In certain aspects, an architectural coating is a high performance architectural coating (“HIPAC”). A HIPAC is architectural coatings that produce a film with a combination of good abrasion resistance, staining resistance, chemical resistance, detergent resistance, and mildew resistance. Examples of binders suitable for producing a HIPAC include a two-pack epoxide or urethane, or a moisture cured urethane. In general embodiments, an architectural coating comprises a liquid component, an additive, or a combination thereof. In certain aspects, an architectural coating is a water-borne coating or a solvent-borne coating. In other aspects, an architectural coating comprises a pigment. In some aspects, such an architectural coating is formulated to comprise a reduced amount or lack a toxic coating component. Examples of a toxic coating component include a heavy metal (e.g., lead), formaldehyde, a nonyl phenol ethoxylate surfactant, a crystalline silicate, or a combination thereof.

In certain embodiments, a water-borne coating has a density of 1.20 kg/L to 1.50 kg/L, including all intermediate ranges and combinations thereof. In other embodiments, a solvent-borne coating has a density of 0.90 kg/L to 1.2 kg/L, including all intermediate ranges and combinations thereof. The density of a coating can be empirically determined, for example, as described in “ASTM Book of Standards, Volume 06.01, Paint—Tests for Chemical, Physical, and Optical Properties; Appearance,” D1475-98, 2002. In certain embodiments, the course particle content of an architectural coating, by weight, is 0.5% or less. The coarse particle (e.g., coarse contaminants, pigment agglomerates) content of a coating can be empirically determined, for example, as described in “ASTM Book of Standards, Volume 06.03, Paint—Pigments, Drying Oils, Polymers, Resins, Naval Stores, Cellulosic Esters, and Ink Vehicles,” D185-84, 2002. In some embodiments, the viscosity for an architectural coating at relatively low shear rates used during typical application, in Krebs Units (“Ku”), is 72 Ku to 95 Ku, including all intermediate ranges and combinations thereof.

In typical use, an architectural coating is often stored in a container for months or even years prior to first use, and/or between different uses. In many embodiments, it will be contemplated that a building coating will retain a desirable set properties of a coating, film formation, film, or a combination thereof, for a period of 12 months or greater in a container at ambient conditions. Properties that are contemplated for storage include settling resistance, skinning resistance, coagulation resistance, viscosity alteration resistance, or a combination thereof. Storage properties can be empirically determined for a coating (e.g., an architectural coating) as described, for example, in “ASTM Book of Standards, Volume 06.02, Paint—Products and Applications; Protective Coatings; Pipeline Coatings,” D869-85 and D1849-95, 2002.

It is contemplated that application and/or film formation of an architectural coating occurs at ambient conditions to provide ease of use to a casual user of the coating, as well as reduce potential damage to the target surface and the surrounding environment (e.g., unprotected people and objects). In general embodiments, it is contemplated that an architectural coating does not undergo film formation by a temperature greater than 40° C. to reduce possible heat and fire damage. In other embodiments, it is contemplated that an architectural coating is suitable to be applied by using hand-held applicator. Hand-held applicators are generally can be used without difficulty by many users of a coating, and examples include a brush, a roller, a sprayer (e.g., a spray can), or a combination thereof.

Specific procedures for determining the suitability of a coating and/or film for use as an architectural coating (e.g., a water-borne coating, a solvent-borne coating, an interior coating, an exterior paint, a latex paint), and specific assays for properties typically desired in an architectural coating (e.g., blocking resistance, hiding power, print resistance, washability, weatherability, corrosion resistance) have been described, for example, in “ASTM Book of Standards, Volume 06.02, Paint—Products and Applications; Protective Coatings; Pipeline Coatings,” D5324-98, D5146-98, D3730-98, D1848-88, D5150-92, D2064-91, D4946-89, D6583-00, D3258-00, and D3450-00, 2002; “ASTM Book of Standards, Volume 06.01, Paint—Tests for Chemical, Physical, and Optical Properties; Appearance,” D660-93, D4214-98, D772-86, D662-93, and D661-93, 2002; and in “Paint and Coating Testing Manual, Fourteenth Edition of the Gardner-Sward Handbook” (Koleske, J. V. Ed.), pp. 696-705, 1995.

(1) Wood Coatings

A wood coating is often selected to protect the wood from damage, as well as aesthetic purposes. For example, wood is susceptible to damage from bacteria and fungi. Examples of fungi that damage wood include Aureobasidium pullulans, and Ascomycotina, Deutermycotina, Basidiomycetes, Coniophora puteana, Serpula lac ymans, and Dacymyces stillatus. In some embodiments, a wooden surface is impregnated with a preservative such as a fungicide, prior to application of a coating. However, much of the wood that is contemplated as a surface for a coatingis provided this way from wood suppliers. Specific procedures for determining the presence of a preservative and/or water repellent in wood have been described, for example, in “ASTM Book of Standards, Volume 06.02, Paint—Products and Applications; Protective Coatings; Pipeline Coatings,” D2921-98, 2002.

Typically, wood surfaces are coated with a paint, a varnish, a stain, or a combination thereof. Often, the choice of coating is based on the ability of a coating to protect the wood from damage by moisture. Generally, a paint, a varnish, and a stain generally have progressively greater permeability to moisture, and moisture penetration of a wooden surface can which can lead to undesirable alterations in wood structure (e.g., splitting); undesirable alteration in piece of wood's dimension (“dimensional movement”) such as shrinking, swelling, and/or warping; promote the growth of a microorganism such as fungi (e.g., wet rot, dry rot); or a combination thereof. Additionally, UV light irradiation damages a wood surface by depolymerizing lignin comprised in the wood. It is contemplated that in embodiments wherein a wood surface is irradiated by UV light (e.g., sunlight), the wood coating comprises a UV protective agent such as a pigment that absorbs UV light. An example of a UV absorbing pigment includes a transparent iron oxide.

In specific embodiments, a paint for use on a wood surface comprises an oil-paint, an alkyd-paint, or a combination thereof. A type of alkyd-paint for use on a wood surface comprises a solvent-borne paint. In some embodiments, a paint system comprises a combination of a primer, an undercoat, and a topcoat. A film produced by a paint is often moisture impermeable. A film produced by paint upon a wooden surface may crack, flake, trap moisture that can encourage wood decay, be expensive to repair, or a combination thereof.

(2) Masonry Coatings

Masonry coatings refer to coatings used on a masonry surface, such as, for example, stone, brick, tile, cement-based materials (e.g., concrete, mortar), or a combination thereof. In general embodiments, a masonry coating is selected to confer resistance to water (e.g., salt water), resistance to acid conditions, alteration of appearance (e.g., color, brightness), or a combination thereof. Typically, a masonry coating comprises a multicoat system. In specific embodiments, a masonry multicoat system comprises a primer, a topcoat, or a combination thereof. Examples of a masonry primer include a rubber primer (e.g., a styrene-butadiene copolymer primer). In certain embodiments, a topcoat comprises a water-borne coating or a solvent borne coating. Examples of a water-borne coating that may be selected for a masonry topcoat include a latex coating, a water reducible polyvinyl acetate-coating, or a combination thereof. In certain aspects, a solvent-borne topcoat comprises a thermoplastic coating, a thermosetting coating, or a combination thereof. Examples of a thermosetting coating include an oil, an alkyd, a urethane, an epoxy, or a combination thereof. In certain aspects, a thermosetting coating is a multi-pack coating, such as, for example, an epoxy, a urethane, or a combination thereof. In specific aspects, a thermosetting coating undergoes film formation at ambient conditions. In other aspects, a thermosetting coating undergoes film formation at film formation at an elevated temperature such as a baking alkyd, a baking acrylic, a baking urethane, or a combination thereof. Examples of a thermoplastic coating include an acrylic, cellulosic, a rubber-derivative, a vinyl, or a combination thereof. In specific aspects, a thermoplastic coating is a lacquer.

A masonry surface that is basic in pH, such as, for example, cement-based material and/or a calcareous stone (e.g., marble, limestone) may be damaging to certain coatings. Specific procedures for determining the pH of a masonry surface have been described, for example, in “ASTM Book of Standards, Volume 06.02, Paint—Products and Applications; Protective Coatings; Pipeline Coatings,” D4262, 2002. Due to porosity and/or contact with an external environment, a masonry surface often accumulates dirt and other loose surface contaminants, which typically are removed prior to application of a coating. Specific procedures for preparative cleaning (e.g., abrading, acid etching) of a masonry surface (e.g., sandstone, clay brick, concrete) have been described, for example, in “ASTM Book of Standards, Volume 06.02, Paint—Products and Applications; Protective Coatings; Pipeline Coatings,” D4259-88, D4260-88D, 5107-90, D5703-95, D4261-83, and D4258-83, 2002. In certain embodiments, moisture at or near a masonry surface may be undesirable during application of a coating (e.g., a solvent-borne coating). Specific procedures for determining the presence of such moisture upon a masonry surface have been described, for example, in “ASTM Book of Standards, Volume 06.02, Paint—Products and Applications; Protective Coatings; Pipeline Coatings,” D4263-83, 2002. Specific procedures for determining the suitability of a coating and/or film, particularly in conferring water resistance to a masonry surface, have been described, for example, in “ASTM Book of Standards, Volume 06.02, Paint—Products and Applications; Protective Coatings; Pipeline Coatings,” D6237-98, D4787-93, D5860-95, D6489-99, D6490-99, and D6532-00, 2002. Additional procedures for determining the suitability of a coating and/or film for use as a masonry coating have been described, for example, in “Paint and Coating Testing Manual, Fourteenth Edition of the Gardner-Sward Handbook,” (Koleske, J. V. Ed.), pp. 725-730, 1995.

(3) Artist's Coatings

Artist coatings refer to a coating used by artists for a decorative purpose. Often, an artist's coating (e.g., paint) is selected for durability for decades or centuries at ambient conditions, usually indoors. Coatings such as an alkyd coating, an oil coating, an oleoresinous coating, an emulsion (e.g., acrylic emulsion) coating, or a combination thereof, are typically selected for use as an artist's coating. Specific standards for physical properties, chemical properties, and/or procedures for determining the suitability (e.g., lightfastness) of a coating and/or film for use as an artist's coating have been described, for example, in “ASTM Book of Standards, Volume 06.02, Paint—Products and Applications; Protective Coatings; Pipeline Coatings,” D4236-94, D5724-99, D4302-99, D4303-99, D4941-89, D5067-99, D5098-99, D5383-02, D5398-97, D5517-00, and D6801-02a, 2002; and in “Paint and Coating Testing Manual, Fourteenth Edition of the Gardner-Sward Handbook,” (Koleske, J. V. Ed.), pp. 706-710, 1995.

b. Industrial Coatings

An industrial coating is a coating applied to a surface of a manufactured product in a factory setting. An industrial coating typically undergoes film formation to produce a film with a protective and/or aesthetic purpose. Industrial coatings share some similarities to an architectural coating, such as comprising similar coating components, being applied to the same material types of surfaces, being applied to an interior surface, being applied to an exterior surface, or a combination thereof. Examples of coating types that are commonly used for an industrial coating include an epoxy-coating, a urethane-coating, alkyd-coating, a vinyl-coating, chlorinated rubber-coating, or a combination thereof. Examples of a surface commonly coated by an industrial coating include metal (e.g., aluminum, zinc, copper, an alloy, etc); glass; plastic; cement; wood; paper; or a combination thereof. An industrial coating may be storage stable for 12 months or more, applied at ambient conditions, applied using a hand-held applicator, undergo film formation at ambient conditions, or a combination thereof.

However, an industrial coating often does not meet one or more of these characteristics previously described for an architectural coating. For example, an industrial coating may have a storage stability of days, weeks, or months, as due to a more rapid use rate in coating factory prepared items. An industrial coating may be applied and/or undergo film formation at baking conditions. An industrial coating may be applied using techniques such as, for example, spraying by a robot, anodizing, electroplating, and/or laminating of a coating and/or film onto a surface. In some embodiments, an industrial coating undergoes film formation by irradiating the coating with non-visible light electromagnetic radiation and/or particle radiation such as UV radiation, infrared radiation, electron-beam radiation, or a combination thereof.

In certain embodiments, an industrial coating comprises an industrial maintenance coating, which is a coating that produces a protective film with excellent heat resistance (e.g., 121° C. or greater), solvent resistance (e.g., an industrial solvent, an industrial cleanser), water resistance (e.g., salt water, acidic water, alkali water), corrosion resistance, abrasion resistance (e.g., mechanical produced wear), or a combination thereof. An example of an industrial maintenance coating includes a high-temperature industrial maintenance coating, which is applied to a surface intermittently or continuously contacted with a temperature of 204° C. or greater. An additional example of an industrial maintenance coating is an industrial maintenance anti-graffiti coating, which is a two-pack clear coating applied to an exterior surface that is intermittently contacted with a solvent and abrasion. Examples of coating types that are commonly used for an industrial maintenance coating include an epoxy-coating, a urethane-coating, alkyd-coating, a vinyl-coating, chlorinated rubber-coating, or a combination thereof.

Industrial coatings (e.g., coil coatings) and their use have been described in the art (see, for example, in “Paint and Surface Coatings: Theory and Practice,” 2^{nd} Edition, pp. 502-528, 1999; in “Paints, Coatings and Solvents,” 2^{nd} Edition, pp. 330-410, 1998; in “Organic Coatings: Science and Technology, Volume 1: Film Formation, Components, and Appearance,” 2^{nd} Edition, pp. 138, 317-318). Standard procedures for determining the properties of an industrial coating (e.g., an industrial wood coating, an industrial water-reducible coating) have been described, for example, in “ASTM Book of Standards, Volume 06.02, Paint—Products and Applications; Protective Coatings; Pipeline Coatings,” D4712-87a, D6577-00a, D2336-99, D3023-98, D3794-00, D4147-99, and D5795-95, 2002.

(1) Automotive Coatings

Automotive coatings refer to coatings used on automotive vehicles, particularly those for civilian use. The manufacturers of a vehicle typically require that a coating conform to specific properties of weatherability (e.g., UV resistance) and/or appearance. Typically, an automotive coating comprises a multicoat system. In specific embodiments, an automotive multicoat system comprises a primer, a topcoat, or a combination thereof. Examples of an automotive primer include a nonweatherable primer, which lack sufficient UV resistance for single layer use, or a weatherable primer, which possesses sufficient UV resistance to be used without an additional layer. Examples of a topcoat include an interior topcoat, an exterior topcoat, or a combination thereof.

Examples of a nonweatherable automotive primer include a primer applied by electrodeposition, a conductive (“electrostatic”) primer, or a nonconductive primer. In certain embodiments, a primer is applied by electrodeposition, wherein a metal surface is immersed in a primer, and electrical current promotes application of a primer component (e.g., a binder) to the surface. An example of a metal primer suitable for electrodeposition application includes a primer comprising an epoxy binder comprising an amino moiety, a blocked isocyanate urethane binder, and a 75% to 95% aqueous liquid component. In other embodiments, a primer is a conductive primer, which allows additional coating layers to be applied using electrostatic techniques. A conductive primer typically is applied to a plastic surface, including a flexible plastic surface or a nonflexible plastic surface. Such primers vary in their respective flexibility property to better suit use upon the surface. An example of a flexible plastic conductive primer includes a primer comprising polyester binder, a melamine binder and a conductive carbon black pigment. An example of a nonflexible plastic primer includes a primer that comprises an epoxy ester binder and/or an alkyd binder, a melamine binder and conductive carbon black pigment. In certain embodiments, a melamine binder may be partly or fully replaced with an aromatic isocyanate urethane binder, wherein the coating is a two-pack coating. A nonconductive primer is similar to a conductive primer, except the carbon-black pigment is absent or reduced in content. In certain embodiments, a nonconductive primer is a metal primer, a plastic primer, or a combination thereof. In specific aspects, the nonconductive primer comprises a pigment for colorizing purposes.

Examples of a weatherable automotive primer include a primer/topcoat or a conductive primer. An example of a primer/topcoat includes a flexible plastic primer, with suitable weathering properties (e.g., UV resistance) to function as a single layer topcoat. Examples of a flexible plastic primer include a primer comprising an acrylic and/or polyester binder and a melamine binder. In certain embodiments, a melamine binder may be partly or fully replaced with an aliphatic isocyanate urethane binder, wherein the coating is a two-pack coating. A weatherable conductive primer typically is similar to a weatherable primer/topcoat, including a conductive pigment. In specific aspects, a weatherable automotive primer comprises a pigment for colorizing purposes.

An interior automotive topcoat typically is applied to a metal surface, a plastic surface, a wood surface, or a combination thereof. In certain aspects, an interior automotive topcoat is part of a multicoat system further comprising a primer. Examples of an interior automotive topcoat include a coating comprising a urethane binder, an acrylic binder, or a combination thereof.

An exterior automotive topcoat is typically applied to a metal surface, a plastic surface, or a combination thereof. In certain aspects, an exterior automotive topcoat is part of a multicoat system further comprising a primer, sealer, undercoat, or a combination thereof. In certain embodiments, an exterior automotive topcoat comprises a binder capable of thermosetting in combination with a melamine binder. Examples of such a thermosetting binder include an acrylic binder, an alkyd binder, a urethane binder, polyester binder, or a combination thereof. In certain embodiments, a melamine binder may be partly or fully replaced with a urethane binder, wherein the coating is a two-pack coating. In typical embodiments, an exterior automotive topcoat further comprises a light stabilizer, a UV absorber, or a combination thereof. In general aspects, an exterior automotive topcoat further comprises a pigment.

Specific procedures for determining the suitability of a coating (e.g., a nonconductive coating) and/or film for use as an automotive coating, including spray application suitability, coating VOC content and film properties (e.g., corrosion resistance, weathering) have been described, for example, in “ASTM Book of Standards, Volume 06.01, Paint—Tests for Chemical, Physical, and Optical Properties; Appearance,” D5087-02, D6266-00, and D6675-01, 2002; and “ASTM Book of Standards, Volume 06.02, Paint—Products and Applications; Protective Coatings; Pipeline Coatings,” D5066-91, D5009-02, D5162-01, and D6486-01, 2002; and in “Paint and Coating Testing Manual, Fourteenth Edition of the Gardner-Sward Handbook,” (Koleske, J. V. Ed.), pp. 711-716, 1995.

(2) Can Coatings

Can coatings refer to coatings used on a container (e.g., an aluminum container, a steel container), for food, chemicals, or a combination thereof. The manufacturers of a can typically require that a coating conform to specific properties of corrosion resistance, inertness (e.g., to prevent flavor alterations in food, a chemical reaction with a container's contents, etc), appearance, durability, or a combination thereof. Typically, a can coating comprises an acrylic-coating, an alkyd-coating, an epoxy-coating, a phenolic-coating, a polyester-coating, a poly(vinyl chloride)-coating, or combination thereof. Though a can may be made of the same or similar material, different surfaces of a can may require coatings of differing properties of inertness, durability and/or appearance. For example, a coating for a surface of the interior of a can that contacts the container's contents may be selected for a chemical inertness property, a coating for a surface at the end of a can may be selected for a physical durability property, or a coating for a surface on the exterior of a can may be selected for an aesthetic property. To meet the varying can surface requirements, a can coating may comprise a multicoat system. In specific embodiments, a can multicoat system comprises a primer, a topcoat, or a combination thereof. In certain embodiments, an epoxy-coating, a poly(vinyl chloride-coating), or a combination thereof is selected as a primer for a surface at the end of a can. In other embodiments, an oleoresinous-coating, a phenolic-coating, or a combination thereof is selected as a primer for a surface in the interior of a can. In some aspects, a water-borne epoxy and acrylic-coating is selected as a topcoat for a surface of an interior of a can. In additional embodiments, an acrylic-coating, an alkyd-coating, a polyester-coating, or a combination thereof is selected as an exterior coating. In certain facets, a can coating (e.g., a primer, a topcoat) will further comprise an amino resin, a phenolic resin, or a combination thereof for cross-linking in a thermosetting film formation reaction. In certain embodiments, a can coating is applied to a surface by spray application. In other embodiments, a can coating undergoes film formation by UV irradiation. Specific procedures for determining the suitability of a coating and/or film for use as a can coating, have been described, for example, in “Paint and Coating Testing Manual, Fourteenth Edition of the Gardner-Sward Handbook,” (Koleske, J. V. Ed.), pp. 717-724, 1995.

(3) Sealant Coatings

Sealant coatings refer to coatings used to fill a joint to reduce or prevent passage of a gas (e.g., air), water, a small material (e.g., dust), a temperature change, or a combination thereof. A sealant coating (“sealant”) may be thought of as a coating that bridges by contact two or more surfaces. A joint is a gap or opening between two or more surfaces, which may or may not be of the same material type (e.g., metal, wood, glass, masonry, plastic, etc). In typical embodiments, a joint has a width, depth, breadth, or a combination thereof, of 0.64 mm to 5.10 mm, including all intermediate ranges and combinations thereof.

In certain embodiments, a sealant coating comprises an oil, a butyl, an acrylic, a blocked styrene, a polysulfide, a urethane, a silicone, or a combination thereof. A sealent may be a solvent-borne coating or a water-borne coating (e.g., a latex). In certain aspects, a sealant comprises a latex (e.g., an acrylic latex). In other embodiments, a sealant is selected for flexibility, as one or more of the joint surfaces may move during normal use. Examples of a flexible sealant include a silicone, a butyl, an acrylic, a blocked styrene, an acrylic latex, or a combination thereof. An oil sealent typically comprises a drying oil, an extender pigment, a thixotrope, and a drier. A solvent-borne butyl sealent typically comprises a polyisobytylene and/or a polybutene, an extender pigment (e.g., talc, calcium carbonate), a liquid component, and an additive (e.g., an adhesion promoter, an antioxidant, a thixotrope). A solvent-borne acrylic sealent typically comprises a polymethylacrylate (e.g., polyethyl, polybutyl), a colorant, a thixotrope, an additive, and a liquid component. A solvent-borne blocked styrene sealant typically comprises styrene, styrene-butadiene, isoprene, or a combination thereof, and a liquid component. A solvent-borne acrylic sealant, blocked styrene sealant, or a combination thereof typically is selected for aspects wherein UV resistance is desired. A urethane sealant may be a one-pack or two-pack coating. A solvent-borne one-pack urethane sealant typically comprises a urethane that comprises a hydroxyl moiety, a filler, a thixotrope, an additive, an adhesion promoter, and a liquid component. A solvent-borne two-pack urethane sealent typically comprises a polyether that comprises an isocyanate moiety in one-pack and a binder comprising a hydroxyl moiety in a second pack. A solvent-borne two-pack urethane sealent typically also comprises a filler, an adhesion promoter, an additive (e.g., a light stabilizer), or a combination thereof. In certain aspects, a solvent-borne urethane sealent is selected for a sealent with a good abrasion resistance. A polysulfide sealant may be a one-pack or two-pack coating. A solvent-borne one-pack polysulfide sealant typically comprises a urethane that comprises a hydroxyl moiety, a filler, a thixotrope, an additive, an adhesion promoter, and a liquid component. A solvent-borne two-pack polysulfide sealent typically comprises a first pack, which typically comprises a polysulfide, an opacifing pigment, a colorizer (e.g., a pigment), clay, a thixotrope (e.g., a mineral), and a liquid component; and a second pack, which typically comprises a curing agent (e.g., lead peroxide), an adhesion promoter, an extender pigment, and a light stabilizer. A silicone sealant typically comprises a polydimethyllsiloxane and a methyltriacetoxy silane, a methyltrimethoxysilane, a methyltricyclorhexylaminosilane, or a combination thereof. A water-borne acrylic latex sealant typically comprises a thermoplastic acrylic, a filler, a surfactant, a thixotrope, an additive, and a liquid component. Procedures for determining the suitability of a coating and/or film for use as an sealant coating have been described, for example, in “Paint and Coating Testing Manual, Fourteenth Edition of the Gardner-Sward Handbook,” (Koleske, J. V. Ed.), pp. 735-740, 1995.

(4) Marine Coatings

A marine coating is a coating used on a surface that contacts water, or a surface that is part of a structure continually near water (e.g., a ship, a dock, an drilling platform for fossil fuels, etc). Typically, such surfaces comprise metal, such as aluminum, high tensile steel, mild steel, or a combination thereof. For embodiments wherein a surface contacts water, the type of marine coating is selected to resist fouling, corrosion, or a combination thereof. Fouling is an accumulation of aquatic organisms, including microorganisms, upon a marine surface. Fouling can damage a film, and as many marine coatings are formulated with a preservative, an anti-corrosion property (e.g., an anticorrosion pigment), or a combination thereof, as such damage often leads to corrosion of metal surfaces. Additionally, a marine coating may be selected to resist fire, such as a coating applied to a surface of a ship. Further properties that are often desirable for a marine coating include chemical resistance, impact resistance, abrasion resistance, friction resistance, acoustic camouflage, electromagnetic camouflage, or a combination thereof.

To achieve the various properties of a marine coating, often a multicoat system is used. For metal surfaces, a primer known as a blast primer is typically applied to the surface within seconds of blast cleaning. Examples of a blast primer include a polyvinyl butyral (“PVB”) and phenolic resin coating, a two-pack epoxy coating, or a two-pack zinc and ethyl silicate coating. A marine metal surface undercoat or topcoat typically comprises an alkyd coating, a bitumen coating, a polyvinyl coating, or a combination thereof. Marine coatings and their use are known in the art (see, for example, in “Paint and Surface Coatings: Theory and Practice,” 2^{nd} Edition, pp. 529-549, 1999; in “Paints, Coatings and Solvents,” 2^{nd} Edition, pp. 252-258, 1998; in “Organic Coatings: Science and Technology, Volume 1: Film Formation, Components, and Appearance,” 2^{nd} Edition, pp. 138, 317-318). Specific procedures for determining the purity/properties of a marine coating, anti-fouling coating, or coating component thereof (e.g., cuprous oxide, copper powder, organotin) under marine conditions (e.g., submergence, water based erosion, seawater biofouling resistance, barnacle adhesion resistance) and/or film have been described, for example, in “ASTM Book of Standards, Volume 06.02, Paint—Products and Applications; Protective Coatings; Pipeline Coatings,” D3623-78a, D4938-89, D4939-89, D5108-90, D5479-94, D6442-99, D6632-01, D4940-98, and D5618-94, 2002; and “ASTM Book of Standards, Volume 06.03, Paint—Pigments, Drying Oils, Polymers, Resins, Naval Stores, Cellulosic Esters, and Ink Vehicles,” D912-81 and D964-65, 2002.

c. Specification Coatings

It is contemplated that, in light of the present disclosures, a specification coating may be formulated by selection of coating components fulfill a set of requirements prescribed by a consumer. Examples a specification finish coating include a military specified coating, a Federal agency specified coating (e.g., Department of Transportation), a state specified coating, or a combination thereof. A specification coating such as a Chemical agent resistant coatings (“CARC”), a camouflage coating, or a combination thereof would be selected in certain embodiments for incorporation of a biomolecular composition. A camouflage coating is a coating that is formulated with materials (e.g., pigments) that reduce the visible differences between the appearance of a coated surface from the surrounding environment. Often, a camouflage coating is formulated to reduce the detection of a coated surface by a devise that measures nonvisible light (e.g., infrared radiation). Various sources of specification coating requirements are described in, for example, “Paint and Coating Testing Manual, Fourteenth Edition of the Gardner-Sward Handbook,” (Koleske, J. V. Ed.), pp. 891-893, 1995).

(1) Pipeline Coatings

An example of a specification coating is a pipeline (e.g., a metal pipeline) coating used to convey a fossil fuel. A pipeline coating should possess corrosion resistance, and an example of a pipeline coating includes a coal tar-coating, a polyethylene-coating, an epoxy powder-coating, or a combination thereof. A coal tar-coating may comprise, for example, a coal tar mastic-coating, a coal tar epoxide-coating, a coal tar urethane-coating, a coal tar enamel-coating, or a combination thereof. A coal tar mastic-coating typically comprises an extender, a vicosifier, or a combination thereof. In general aspects, a coal tar mastic-coating layer is 127 mm to 160 mm thick, including all intermediate ranges and combinations thereof. In embodiments wherein superior water resistance is desired, a coal tar epoxide-coating may be selected. In embodiments wherein rapid film formation is desired (e.g., pipeline repair), a coal tar urethane-coating may be selected. In embodiments wherein good water resistance, heat resistance up to 82° C., bacterial resistance, poor UV resistance, or a combination thereof, is suitable, a coal tar enamel may be selected. In embodiments wherein cathodic protection, physical durability, or a combination thereof is desired, an epoxide powder-coating may be selected. In certain embodiments, an electrostatic spray applicator may be used to apply the powder coating. In certain embodiments, a pipeline coating comprises a multicoat system. In specific aspects, a pipeline multicoat system comprises an epoxy powder primer, a two-pack epoxy primer, a chlorinated rubber primer, or a combination thereof and a polyethylene topcoat. Specific procedures for determining the suitability of a coating and/or film for use as a pipeline coating, including coating storage stability (e.g., settling) and film properties (e.g., abrasion resistance, water resistance, flexibility, weathering, film thickness, impact resistance, chemical resistance, cathodic disbonding resistance, heat resistance) have been described, for example, in “ASTM Book of Standards, Volume 06.02, Paint—Products and Applications; Protective Coatings; Pipeline Coatings,” G6-88, G9-87, G10⁻⁸³, G11-88, G12-83, G13-89, G20-88, G70-81, G8-96, G17-88, G18-88, G19-88, G42-96, G55-88, G62-87, G80-88, G95-87, and D6676-01e1, 2002; and in “Paint and Coating Testing Manual, Fourteenth Edition of the Gardner-Sward Handbook,” (Koleske, J. V. Ed.), pp. 731-734, 1995.

(2) Traffic Marker Coatings

A traffic marker coating is a coating (e.g., a paint) used to visibly convey information on a surface usually subjected to weathering and abrasion (e.g., a pavement). A traffic marker coating may be a solvent-borne coating or a water-borne coating. Examples of a solvent-borne traffic marker coating include an alkyd, a chlorinated rubber, or a combination thereof. In certain aspects, a solvent-borne coating is applied by spray application. In some embodiments, a traffic marker coating is a two-pack coating, such as, for example, an epoxy-coating, a polyester-coating, or a combination thereof. In other embodiments, a traffic marker coating comprises a thermoplastic coating, a thermosetting coating, or a combination thereof. Examples of a combination thermoplastic/thermosetting coating include a solvent-borne alkyd and/or solvent-borne chlorinated rubber-coating. Examples of a thermoplastic coating include a maleic-modified glycerol ester-coating, a hydrocarbon-coating, or a combination thereof. In certain aspects, a thermoplastic coating comprises a liquid component, wherein the liquid component comprises a plasticizer, a pigment, and an additive (e.g., a glass bead).

Specific procedures for determining the suitability of a coating and/or film for use as a traffic marker paint, including coating storage stability (e.g., settling), glass bead properties (e.g., reflectance), film durability (e.g., adhesion, pigment retention, solvent resistance, fuel resistance) and particularly relevant film visual properties (e.g., retroreflectance, fluorescence) have been described, for example, in “ASTM Book of Standards, Volume 06.02, Paint—Products and Applications; Protective Coatings; Pipeline Coatings,” D713-90, D868-85, D969-85, D1309-93, D2205-85, D2743-68, D2792-69, D4796-88, D4797-88, D1155-89, D1214-89, and D4960-89, 2002; in “ASTM Book of Standards, Volume 06.01, Paint—Tests for Chemical, Physical, and Optical Properties; Appearance,” F923-00, E1501-99e1, E1696-02, E1709-00e1, E1710-97, E1743-96, E2176-01, E808-01, E809-02, E810-01, E811-95, D4061-94, E2177-01, E991-98, and E1247-92, 2002; and in “Paint and Coating Testing Manual, Fourteenth Edition of the Gardner-Sward Handbook,” (Koleske, J. V. Ed.), pp. 741-747, 1995.

(3) Aircraft Coatings

An aircraft coating protects and/or decorates a surface (e.g., metal, plastic) of an aircraft. Typically, an aircraft coating is selected for excellent weathering properties, excellent heat and cold resistance (e.g., −54° C. to 177° C.), or a combination thereof. Specific procedures for determining the suitability of a coating and/or film for use as aircraft coating, are described in, for example, in “Paint and Coating Testing Manual, Fourteenth Edition of the Gardner-Sward Handbook,” (Koleske, J. V. Ed.), pp. 683-695, 1995.

(4) Nuclear Power Plant Coatings

An additional example of a specification coating is a coating for a nuclear power plant, which generally should possess particular properties (e.g., gamma radiation resistance, chemical resistance), as described in “ASTM Book of Standards, Volume 06.02, Paint—Products and Applications; Protective Coatings; Pipeline Coatings,” D5962-96, D5163-91, D5139-90, D5144-00, D4286-90, D3843-00, D3911-95, D3912-95, D4082-02, D4537-91, D5498-01, and D4538-95, 2002.

### J. Coating Components

In addition to the disclosures herein, the preparation and/or chemical syntheses of coating components, other than the biomolecular compositionhave been described [see, for example, “Paint and Coating Testing Manual, Fourteenth Edition of the Gardner-Sward Handbook,” (Koleske, J. V., Ed.) (1995); “Paint and Surface Coatings: Theory and Practice, Second Edition,” (Lambourne, R. and Strivens, T. A., Eds.) (1999); Wicks, Jr., Z. W., Jones, F. N., Pappas, S. P. “Organic Coatings, Science and Technology, Volume 1: Film Formation, Components, and Appearance,” (1992); Wicks, Jr., Z. W., Jones, F. N., Pappas, S. P. “Organic Coatings, Science and Technology, Volume 2: Applications, Properties and Performance,” (1992); “Paints, Coatings and Solvents, Second, Completely Revised Edition,” (Stoye, D. and Freitag, W., Eds.) (1998); “Handbook of Coatings Additives,” 1987; In “Waterborne Coatings and Additives” 1995; “ASTM Book of Standards, Volume 06.01, Paint—Tests for Chemical, Physical, and Optical Properties; Appearance,” (2002); “ASTM Book of Standards, Volume 06.02, Paint—Products and Applications; Protective Coatings; Pipeline Coatings,” (2002); “ASTM Book of Standards, Volume 06.03, Paint—Pigments, Drying Oils, Polymers, Resins, Naval Stores, Cellulosic Esters, and Ink Vehicles,” (2002); and “ASTM Book of Standards, Volume 06.04, Paint—Solvents; Aromatic Hydrocarbons,” (2002)].

However, coating components are typically obtained from commercial vendors, which is a method of obtaining a coating component commonly used due to ease and reduced cost. Various texts, for example, Flick, E. W. “Handbook of Paint Raw Materials, Second Edition,” 1989, describes over 4,000 coating components (e.g., an antifoamer, an antiskinning agent, a bactericide, a binder, a defoamer, a dispersant, a drier, an extender, a filler, a flame/fire retardant, a flatting agent, a fungicide, a latex emulsion, an oil, a pigment, a preservative, a resin, a rheological/viscosity control agent, a silicone additive, a surfactant, a titanium dioxide, etc) provided by commercial vendors; and Ash, M. and Ash, I. “Handbook of Paint and Coating Raw Materials, Second Edition,” 1996, which describes over 18,000 coating components (e.g., an accelerator, an adhesion promoter, an antioxidant, an antiskinning agent, a binder, a coalescing agent, a defoamer, a diluent, a dispersant, a drier, an emulsifier, a fire retardant, a flow control agent, a gloss aid, a leveling agent, a marproofing agent, a pigment, a slip agent, a thickener, a UV stabilizer, viscosity control agent, a wetting agent, etc) provided by commercial vendors.

Specific commercial vendors are referred to herein as examples, and include Acima™ AG, Im Ochsensand, CH-9470 Buchs/SG; Air Products and Chemicals, Inc., 7201 Hamilton Boulevard, Allentown, Pa. 18195-1501; Arch Chemicals, Inc., 350 Knotter Drive, Cheshire, Conn., 06410 U.S.A.; Avecia Inc., 1405 Foulk Road, PO Box 15457, Wilmington, Del. 19850-5457, U.S.A.; Bayer Corporation, 100 Bayer Rd., Pittsburgh, Pa. 15205-9741, U.S.A.; Buckman Laboratories, Inc., 1256 North McLean Blvd., Memphis, Tenn. 38108-0305, U.S.A.; BYK-Chemie GmbH, Abelstrasse 45, P.O. Box 100245, D-46462 Wesel, Germany; Ciba Specialty Chemicals, 540 White Plains Road, P.O. Box 2005, Tarrytown, N.Y. 10591-9005, U.S.A.; Clariant LSM (America) Inc., 200 Rodney Building, 3411 Silverside Road, Wilmington, Del. 19810 U.S.A.; Cognis Corporation, 5051 Estecreek Drive, Cincinnati, Ohio 45232-1446, U.S.A.; Condea Servo LLC., 4081 B Hadley Road, South Plainfield, N.J. 07080-1114, U.S.A.; Cray Valley Limited, Waterloo Works, Machen, Caerphilly CF838YN United Kingdom; Dexter Chemical L.L.C., 845 Edgewater Road, Bronx, N.Y. 10474, U.S.A.; Dow Chemical Company, 2030 Dow Center, Midland, Mich. 48674 U.S.A.; Elementis Specialties, Inc., PO Box 700, 329 Wyckoffs Mill Road, Hightstown, N.J. 08520 U.S.A.; Goldschmidt Chemical Corp., 914 East Randolph Road PO Box 1299 Hopewell, Va. 23860 U.S.A.; Hercules Incorporated, 1313 North Market Street, Wilmington, Del. 19894-0001, U.S.A.; International Specialty Products, 1361 Alps Road, Wayne, N.J. 07470, U.S.A.; Octel-Starreon LLC USA, North American Headquarters, 8375 South Willow Street, Littleton, Colo. 80124, U.S.A.; Rohm and Haas Company, 100 Independence Mall West, Philadelphia, Pa. 19106-2399, U.S.A.; Solvay Advanced Functional Minerals, Via Varesina 2-4, 1-21021 Angera (VA); Troy Corporation, 8 Vreeland Road, PO Box 955, Florham Park, N.J., 07932 U.S.A.; R. T. Vanderbilt Company, Inc., 30 Winfield Street, Norwalk, Conn. 06855, U.S.A; Union Carbide Chemicals and Plastics Co., Inc., 39 Old Ridgebury Road, Danbury, Conn. 06817-0001, U.S.A.

1. Binders

A binder (“polymer,” “resin,” “film former”) is a molecule capable of film formation. Film formation is a physical and/or chemical change of a binder in a coating, wherein the change converts the coating into a film. Often, a binder converts into a film through a polymerization reaction, wherein a first binder molecule covalently bonds with at least a second binder molecule to form a larger molecule, known as a “polymer.” As this process is repeated a plurality of times, the composition converts from a coating comprising a binder into a film comprising a polymer.

A binder may comprise a monomer, an oligomer, a polymer, or a combination thereof. A monomer is a single unit of a chemical species that can undergo a polymerization reaction. However, a binder itself is often a polymer, as such larger binder molecules are more suitable for formulation into a coating capable of both being easily applied to a surface and undergoing an additional polymerization reaction to produce a film. An oligomer comprises 2 to 25 polymerized monomers, including all intermediate ranges and combinations thereof.

A homopolymer is a polymer that comprises monomers of the same chemical species. A copolymer is a polymer that comprises monomers of at least two different chemical species. A linear polymer is an unbranched chain of monomers. A branched polymer is a branched (“forked”) chain of monomers. A network (“cross-linked”) polymer is a branched polymer wherein at least one branch forms an interconnecting covalent bond with at least one additional polymer molecule.

A thermoplastic binder and/or coating reversibly softens and/or liquefies when heated. Film formation for a thermoplastic coating generally comprises a physical process, typically the loss of the volatile (e.g., liquid) component from a coating. As a volatile component is removed, a solid film may be produced through entanglement of the binder molecules. In many aspects, a thermoplastic binder is generally a higher molecular mass than a comparable thermosetting binder. In many aspects, a thermoplastic film is often susceptible to damage by a volatile component that can be absorbed by the film, which can soften and/or physically expand the film. In certain facets, a thermoplastic film may be removed from a surface by use of a volatile component. However, in many aspects, damage to a thermoplastic film may be repaired by application of a thermoplastic coating into the damaged areas and subsequent film formation.

A thermosetting binder undergoes film formation by a chemical process, typically the cross-linking of a binder into a network polymer. In certain embodiments, a thermosetting binder does not possess significant thermoplastic properties.

The glass transition temperature is the temperature wherein the rate of increase of the volume of a binder or a film changes. Binders and films often do not convert from solid to liquid (“melt”) at a specific temperature (“Tₘ”), but rather possess a specific glass transition temperature wherein there is an increase in the rate of volume expansion with increasing temperature. At temperatures above the glass transition temperature, a binder or film becomes increasingly rubbery in texture until it becomes a viscous liquid. In certain embodiments described herein, a binder, particularly a thermoplastic binder, may be selected by its glass transition temperature, which provides guidance to the temperature range of film formation, as well as thermal and/or heat resistance of a film. The lower the T_{g}, the “softer” the resin, and generally, the film produced from such a resin. A softer film typically possesses greater flexibility (e.g., crack resistance) and/or poorer resistance to dirt accumulation than a harder film.

In certain embodiments, a coating comprises a low molecular weight polymer, a high molecular weight polymer, or a combination thereof. Examples of a low molecular weight polymer include an alkyd, an amino resin, a chlorinated rubber, an epoxide resin, an oleoresinous binder, a phenolic resin, a urethane, a polyester, a urethane oil, or a combination thereof. Examples of a high molecular weight polymer include a latex, a nitrocellulose, a non-aqueous dispersion polymer (“NAS”), a solution acrylic, a solution vinyl, or a combination thereof. Examples of a latex include an acrylic, a polyvinyl acetate (“PVA”), a styrene/butadiene, or a combination thereof.

In addition to the disclosures herein, a binder, methods of binder preparation, commercial vendors of binder, and techniques in the art for using an binder in a coating may be used (see, for example, Flick, E. W. “Handbook of Paint Raw Materials, Second Edition,” pp. 287-805 and 879-998, 1989; in “Paint and Coating Testing Manual, Fourteenth Edition of the Gardner-Sward Handbook,” (Koleske, J. V. Ed.), pp. 23-29, 39-67, 74-84, 87, 268-285, 410, 539-540, 732, 735-736, 741, 770, 806-807, 845-849, and 859-861, 1995; in “Paint and Surface Coatings, Theory and Practice, Second Edition,” (Lambourne, R. and Strivens, T. A., Eds.), pp. 2-3, 7-10, 21, 24-40, 40-54, 60-71, 76, 81-86, 352, 358, 381-394, 396, 398, 405, 433-448, 494-497, 500, 537-540, 700-702, and 734, 1999; Wicks, Jr., Z. W., Jones, F. N., Pappas, S. P. “Organic Coatings, Science and Technology, Volume 1: Film Formation, Components, and Appearance,” pp. 39, 49-57, 62, 65-67, 67, 76-80, 83, 91, 104-118, 155, 168, 178, 182-183, 200, 202-203, 209, 214-216, 220 and 250, 162-186, 215-216 and 232, 59-60, 183-184, 133-143, 39, 144-161, 203, 219-220 and 239, 23, 110, 120-132, 122-130, 198, 202-203, 209 and 220, 60-62, 83-103, 164-167, 173, 177-178, 184-187, 195, 206, and 216-219, 1992; Wicks, Jr., Z. W., Jones, F. N., Pappas, S. P. “Organic Coatings, Science and Technology, Volume 2: Applications, Properties and Performance,” pp. 13-14, 18-19, 26, 33-34, 36, 41, 57, 77, 92, 95, 116-119, 143-145, 156, 161-165, 179-180, 191-193, 197-203, 210-211, 213-214, 216, 219-222, 230-239, 260-263, 269-271, 276-284, 288-293, 301-307, 310, 315-316, 319-321, and 325-346, 1992; and in “Paints, Coatings and Solvents, Second, Completely Revised Edition,” (Stoye, D. and Freitag, W., Eds.) pp. 5, 11-22, 37-50, 54-55, 72, 80-87, 96-98, 108, 126, and 136, 1998.

a. Oil-Based Binders

Certain binders, such as, for example, an oil (e.g., a drying oil), an alkyd, an oleoresinous binder, a fatty acid epoxide ester, or a combination thereof, are prepared and/or synthesized from an oil and/or a fatty acid, and undergo film formation by thermosetting oxidative cross-linking of fatty acids, and will be referred to herein as an “oil-based binder.” These types of binders often possess similar properties (e.g., solubility, viscosity). An oil-based binder coating often further comprises a drier, an antiskinning agent, an alkylphenolic resin, a pigment, an extender, a liquid component (e.g., a solvent), or a combination thereof. A drier, such as a primary drier, secondary drier, or a combination thereof, may be selected to promote film formation. In certain facets, an oil-based binder coating may comprise an anti-skinning agent, which is typically used to control undesirable film-formation caused by a primary drier and/or oxidation. A liquid component may be selected, for example, to alter a rheological property (e.g., flow), wetting and/or dispersion of particulate material, or a combination thereof. In certain embodiments, a liquid component comprises a hydrocarbon. In particular embodiments, the hydrocarbon comprises an aliphatic hydrocarbon, an aromatic hydrocarbon (e.g., toluene, xylene), or a combination thereof. In some facets, the liquid component comprises, by weight, 5% to 20% of an oil-based binder coating, including all intermediate ranges and combinations thereof.

In alternative embodiments, an oil-based temporary coating (e.g., a non-film forming coating) may be produced, for example, by inclusion of an antioxidant, reduction of the amount of a drier, selection of a oil-based binder that comprises fewer or no double bonds, or a combination thereof.

An oil-based binder coating may be selected for embodiments wherein a relatively low viscosity is desired, such as, for example, application to a corroded metal surface, a porous surface (e.g., wood), or a combination thereof, due to the penetration power of a low viscosity coating. In certain facets, it is contemplated that application of an oil-binder coating produces a layer having less than 25 μm on vertical surfaces and 40 μm on horizontal surfaces to reduce shrinkage, wrinkling. Additionally, in aspects wherein the profile of the wood surface is to be retained, such a thin film thickness is contemplated. In specific aspects, an oil-binder coating may be selected as a wood stains, a topcoat, or a combination thereof. In particular facets, a wood stain comprises an oil (e.g., linseed oil) coating, an alkyd, or a combination thereof. Often, wood coating comprises a lightstabilizer (e.g., UV absorber).

(1) Oils

An oil is a polyol esterified to at least one fatty acid. A polyol (“polyalcohol,” “polyhydric alcohol”) is an alcohol comprising more than one hydroxyl moiety per molecule. In certain embodiments, an oil comprises an acylglycerol esterified to one fatty acid (“monacylglycerol”), two fatty acids (“diacylglycerol”), or three fatty acids (“triacylglycerol,” “triglyceride”). Typically, however, an oil will comprise a triacylglycerol. A fatty acid is an organic compound comprising a hydrocarbon chain that includes a terminal carboxyl moiety. A fatty acid may be unsaturated, monounsaturated, and polyunsaturated referring to whether the hydrocarbon chain possess no carbon double bonds, one carbon double bond, or a plurality of carbon double bonds (e.g., 2, 3, 4, 5, 6, 7, or 8 double bonds), respectively.

In typical use in a coating, a plurality of fatty acids forms covalent cross-linking bonds to produce a film in coatings comprising oil binders and/or other binders comprising a fatty acid. Usually oxidation through contact with atmospheric oxygen is used to promote film formation. Exposure to light also enhances film formation. The ability of an oil to undergo film formation by chemical cross-linking is related to the content of chemically reactive double bonds available in its fatty acids. Oils are generally a mixture of chemical species, comprising different combinations of fatty acids esterified to glycerol. The overall types and percentages of particular fatty acids that are comprised in oils affect the ability of the oil to be used as a binder. Oils can be classified as a drying oil, a semi-drying oil, or a non-drying oil depending upon the ability of the oil to cross-link into a dry film without additives (e.g., driers) at ambient conditions and atmospheric oxygen. A drying oil forms a dry film to touch upon cross-linking, a semi-drying oil forms a sticky (“tacky”) film to touch upon cross-linking, while a non-drying oil does not produce a tacky or dry film upon cross-linking. In certain facets, it is contemplated that film-formation of a non-chemically modified oil-binder coating will typically take from 12 hours to 24 hours at ambient conditions, air, and lighting. Procedures for selection and testing of drying oils for a coating are described in, for example, “ASTM Book of Standards, Volume 06.03, Paint—Pigments, Drying Oils, Polymers, Resins, Naval Stores, Cellulosic Esters, and Ink Vehicles,” D555-84, 2002.

Drying oils comprise at least one polyunsaturated fatty acid to promote cross-linking. Polyunsaturated fatty acids (“polyenoic fatty acids”) include, but are not limited to, 7,10,13-hexadecatrienoic (“16:3 n-3”); linoleic [“9,12-octadecadienoic,” “18:2(n-6)”]; γ-linolenic [“6,9,12-octadecatrienoic,” “18:3(n-6)”]; a trienoic 20:3(n-9); dihomo-γ-linolenic [“8,11,14-eicosatrienoic,” “20:3(n-6)”]; arachidonic [“5,8,11,14-eicosatetraenoic,” “20:4(n-6)”]; a licanic, (“4-oxo 9c11t13t-18:3”); 7,10,13,16-docosatetraenoic [“22:4(n-6)”]; 4,7,10,13,16-docosapentaenoic [“22:5(n-6)”]; α-linolenic [“9,12,15-octadecatrienoic,” “18:3(n-3)”]; stearidonic [“6,9,12,15-octadecatetraenoic,” “18:4(n-3)”]; 8,11,14,17-eicosatetraenoic [“20:4(n-3)”]; 5,8,11,14,17-eicosapentaenoic [“EPA,” “20:5(n-3)”]; 7,10,13,16,19-docosapentaenoic [“DPA,” “22:5 (n-3)”]; 4,7,10,13,16,19-docosahexaenoic [“DHA,” “22:6(n-3)”]; 5,8,11-eicosatrienoic [“Mead acid,” “20:3(n-9)”]; taxoleic (“all-cis-5,9-18:2”); pinolenic (“all-cis-5,9,12-18:3”); sciadonic (“all-cis-5,11,14-20:3”); dihomotaxoleic (“7,11-20:2”); cis-9, cis-15 octadecadienoic (“9,15-18:2”); retinoic; or a combination thereof.

Drying oils can be further characterized as non-conjugated or conjugated drying oils depending upon whether their abundant fatty acid comprises a polymethylene-interrupted double bond or a conjugated double bond, respectively. A polymethylene-interrupted double bond is two double bonds separated by two or more methylene moieties. A polymethylene-interrupted fatty acid is a fatty acid comprising such a configuration of double bonds. Examples of polymethylene-interrupted fatty acids include taxoleic, pinolenic, sciadonic, dihomotaxoleic, cis-9, cis-15 octadecadienoic, retinoic, or a combination thereof.

A conjugated double bond is a moiety wherein a single methylene moiety connects pair of carbon chain double bonds. A conjugated fatty acid is a fatty acid comprising such a pair of double bonds. A conjugated double bond is more prone to cross-linking reactions than non-conjugated double bonds. A conjugated diene fatty acid, a conjugated triene fatty acid or a conjugated tetraene fatty acid, possesses two, three or four conjugated double bonds, respectively. An example of a common conjugated diene fatty acid is a conjugated linoleic. Examples of a conjugated triene fatty acid include an octadecatrienoic, a licanic, or a combination thereof. Examples of an octadecatrienoic acid include an α-eleostearic comprising the 9c,11t,13t isomer, a calendic comprising a 8t,10t,12c isomer, a catalpic comprising the 9c, 11t,13c isomer, or a combination thereof. An example of a conjugated tetraene fatty acid is a-parinaric comprising the 9c,11t,13t,15c isomer, and β-parinaric comprising the 9t,11t,13t,15t isomer, or a combination thereof.

Oils for use in coatings are generally obtained from renewable biological sources, such as plants, fish or a combination thereof. Examples of plant oils commonly used in coatings or coating components include cottonseed oil, linseed oil, oiticica oil, safflower oil, soybean oil, sunflower oil, tall oil, rosin, tung oil, or a combination thereof. An example of a fish oil commonly used in coatings or coating components include caster oil. A colder environment generally promotes a higher polyunsaturated fatty acid content in an organism (e.g., sunflowers). Cottonseed oil comprises about 36% saturated fatty acids, 24% oleic, and 40% linoleic. Castor oil comprises about 3% saturated fatty acids, 7% oleic, 3% linoleic, and 87% ricinoleic (“12-hydroxy-9-octadecenoic”). Linseed oil comprises about 10% saturated fatty acids, 20% to 24% oleic (“cis-9-octadecenoic”), 14% to 19% linoleic, and 48% to 54% linolenic. Oiticica oil comprises about 16% saturated fatty acids, 6% oleic, and 78% licanic. Safflower oil comprises about 11% saturated fatty acids, 13% oleic, 75% linoleic, and 1% linolenic. Soybean oil comprises about 14% to 15% saturated fatty acids, 22% to 28% oleic, 52% to 55% linoleic, and 5% to 9% linolenic. Tall oil, which is a product of paper production and generally is not in the form of a triglyceride, often comprises about 3% saturated fatty acids, 30% to 35% oleic, 35% to 40% linoleic, 2% to 5% linolenic, and 10% to 15% of a combination of pinolenic and conjugated linoleic. Rosin is a combination of acidic compounds isolated during paper production, such as, for example, abietic acid, neoabietic acid, dihydroabietic acid, tetraabietic acid, isodextropimaric acid, dextropimaric acid, dehydroabietic acid, and levopimaric acid. Tung oil comprises about 5% saturated fatty acids, 8% oleic, 4% linoleic, 3% linolenic, and 80% α-elestearic. Standards for physical properties, chemical properties, and/or procedures for testing the purity/properties of various oils (e.g., caster, linseed, oiticica, safflower, soybean, sunflower, tall, tung, rosin, dehydrated caster, boiled linseed, a drying oil, a fish oil, a heat-bodied drying oil) for use in a coating are described, for example in “ASTM Book of Standards, Volume 06.03, Paint—Pigments, Drying Oils, Polymers, Resins, Naval Stores, Cellulosic Esters, and Ink Vehicles,” D555-84, D960-02a, D961-86, D234-82, D601-87, D1392-92, D1462-92, D12-88, D1981-02, D5768-95, D3169-89, D260-86, D124-88, D803-02, D1541-97, D1358-86, D1950-86, D1951-86, D1952-86, D1954-86, D1958-86, D464-95, D465-01, D1959-97, D1960-86, D1962-85, D1964-85, D1965-87, D1966-69, D1967-86, D3725-78, D1466-86, D890-98, D1957-86, D1963-85, D5974-00, D1131-97, D1240-02, D889-99, D509-98, D269-97, D1065-96, and D804-02, 2002.

In certain embodiments, an oil comprises a chemically modified oil, which is an oil altered by a reaction thought to promote limited cross-linking. Generally, such a modified oil possesses an altered property, such as a higher viscosity, which may be more suitable for a particular coating application. Examples of a chemically modified oil include a bodied oil, a blown oil, a dimer acid, or a combination thereof A bodied oil (“heat bodied oil,” “stand oil”) is produced, for example, by heating a nonconjugated oil (e.g., 320° C.) or a conjugated oil (e.g., 240° C.) in an chemically unreactive atmosphere to promote limited cross-linking. A blown oil is produced, for example, by passing air through a drying oil at, for example, 150° C. A dimer acid is produced, for example, by acid catalyzed dimerization or oligomerization of a polyunsaturated acid.

In certain embodiments, an oil comprises a synthetic conjugated oil, which is an oil altered by a reaction thought to produce a conjugated double bond in a fatty acid of the oil. Conjugated fatty acids have been produced from nonconjugated fatty acids by alkaline hydroxide catalyzed reactions. However, a synthetic conjugated oil is generally semi-drying in air catalyzed film formation at ambient conditions, and a coating comprising such an oil is typically cured by baking. Additionally richinoleic acid, which is prevalent in castor oil, can be dehydrogenated to produce a mixture of conjugated and non-conjugated fatty acids. Dehydrogenated castor oil comprises about 2% to 4% saturated fatty acids, 6% to 8% oleic, 48% to 50% linoleic, and 40% to 42% conjugated linoleic.

Certain other compounds comprising a fatty acid and polyol are classified herein as an oil for use as a binder such as a high ester oil, a maleated oil, or a combination thereof. A high ester oil comprises a polyol capable of comprising greater than three fatty acid esters per molecule and at least one fatty acid ester. However, a high ester oil comprising four or more fatty acid esters per molecule is contemplated in many embodiments. Examples of such a polyol include a pentaerythritiol, a dipentaerythritiol, a tripentaerythritiol, or a styrene/allyl alcohol copolymer. These high ester oils generally form films more rapidly than acylglycerol based oil, as the opportunity for cross-linking reactions between fatty acids increases with the number of fatty acids attached to a single polyol. A maleated oil is an oil modified by a chemical reaction with maleic anhydride. Maleic acid and an unsaturated or polyunsaturated fatty acid react to produce a fatty acid with additional acid moieties. A maleated oil generally is more hydrophilic and/or has a faster film formation time than a comparative non-maleated oil.

(2) Alkyd Resins

In certain embodiments, a binder can comprise an alkyd resin. In general embodiments, an alkyd-coating may be selected as an architectural coating, a metal coating, a plastic coating, a wood coating, or a combination thereof. In certain aspects, an alkyd coating may be selected for use as a primer, an undercoat, a topcoat, or a combination thereof. In particular aspects, an alkyd coating comprises a pigment, an additive, or a combination thereof.

An alkyd resin comprises a polyester prepared from a polyol, a fatty acid, and a polybasic (“polyfunctional”) organic acid or acid anhydride. An alkyd resin is generally produced by first preparing monoacylpolyol, which is a polyol esterified to one fatty acid. The monoacylpolyol is polymerized by ester linkages with a polybasic acid to produce an alkyd resin of desired viscosity in a solvent. Examples of a polyol include 1,3-butylene glycol; diethylene glycol; dipentaerythritol; ethylene glycol; glycerol; hexylene glycol; methyl glucoside; neopentyl glycol; pentaerythritol; pentanediol; propylene glycol; sorbitol; triethylene glycol; trimethylol ethane; trimethylol propane; trimethylpentanediol; or a combination thereof. In certain aspects, a polyol comprises ethylene glycol; glycerol; neopentyl glycol; pentaerythritol; trimethylpentanediol; or a combination thereof. Examples of a polybasic acid or an acid anhydride include adipic acid, azelaic acid, chlorendic anhydride, citric acid, fumaric acid, isophthalic acid, maleic anhydride, phthalic anhydride, sebacic acid, succinic acid, trimelletic anhydride, or a combination thereof. In certain aspects, a polybasic acid or an acid anhydride comprises isophthalic acid, maleic anhydride, phthalic anhydride, trimelletic anhydride, or a combination thereof. Examples of a fatty acid include abiatic, benzoic, caproic, caprylic, lauric, linoleic, linolenic, oleic, a tertiary-butyl benzoic acid, a fatty acid from an oil/fat (e.g., castor, coconut, cottonseed, tall, tallow), or a combination thereof. In certain aspects, a fatty acid comprises benzoic, a fatty acid from tall oil, or a combination thereof. In specific aspects, an oil is used in the reaction directly as a source of a fatty acid and/or a polyol. Examples of an oil include castor oil, coconut oil, corn oil, cottonseed oil, dehydrated castor oil, linseed oil, safflower oil, soybean oil, tung oil, walnut oil, sunflower oil, menhaden oil, palm oil, or a combination thereof. In some aspects, an oil comprises coconut oil, linseed oil, soybean oil, or a combination thereof.

In addition to the standards and analysis techniques previously described for an oil, standards for physical properties, chemical properties, and/or procedures for testing the purity/properties of various fatty acids (e.g., coconut, corn, cottonseed, dehydrated caster, linseed, soybean, tall oil fatty acids, rosin fatty acids) and a polyol (e.g., pentaerythritol, hexylene glycol, ethylene glycol, diethylene glycol, propylene glycol, dipropylene glycol) and acid anhydrides (e.g., phthalic anhydride, maleic anhydride) for use in an alkyd or other coating components are described, for example, in “ASTM Book of Standards, Volume 06.03, Paint—Pigments, Drying Oils, Polymers, Resins, Naval Stores, Cellulosic Esters, and Ink Vehicles,” D1537-60, D1538-60, D1539-60, D1841-63, D1842-63, D1843-63, D5768-95, D1981-02, D1982-85, D1980-87, D804-02, D1957-86, D464-95, D465-01, D1963-85, D5974-00, D1466-86, D2800-92, D1585-96, D1467-89, and D1983-90, 2002; and in “ASTM Book of Standards, Volume 06.04, Paint—Solvents; Aromatic Hydrocarbons,” D2403-96, D3504-96, D2930-94, D3366-95, D3438-99, D2195-00, D2636-01, D2693-02, D2694-91, D5164-91, D1257-90, and D1258-95, 2002. Further, the composition, properties and/or purity of an alkyd resin and/or a solution comprising an alkyd resin selected for use in a coating such as phthalic anhydride content, isophthalic acid content, unsaponifiable matter content, fatty acid content/identification, polyhydric alcohol content/identification, glycerol, ethylene glycol and/or pentaerythirol content, and silicon content can be empirically determined (see, for example, “ASTM Book of Standards, Volume 06.03, Paint—Pigments, Drying Oils, Polymers, Resins, Naval Stores, Cellulosic Esters, and Ink Vehicles,” D2689-88, D563-88, D2690-98, D2998-89, D1306-88, D1397-93, D1398-93, D2455-89, D1639-90, D1615-60, and D2456-91, 2002).

(i) Oil Length Alkyd Binders

In specific embodiments, an alkyd resin may be selected based on the materials used in its preparation, which typically affect the alkyd's properties. In general aspects, an alkyd resin is often classified and/or selected for use in a particular application by its oil content, as the oil content affects the alkyd resin properties. Oil content is the amount of oil relative to the solvent-free alkyd resin. Based on oil content, an alkyd resin may be classified as a very long oil alkyd resin, a long oil alkyd resin, a medium oil alkyd resin, or a short oil alkyd resin. Generally, the greater the oil content classification of an alkyd resin that is comprised in a coating, the greater the ease of brush application, the slower the rate of film formation, the greater the film's flexibility, the poorer the chemical resistance of the film, the poorer the retention of gloss in exterior environments, or a combination thereof. A short oil alkyd, a medium oil alkyd, a long oil alkyd, and a very long oil alkyd has an oil content range of 1% to 40%, 40% to 60%, 60% to 70%, and 70% to 85%, respectively, including all intermediate ranges and combinations thereof, respectively. In typical embodiments, a short oil alkyd, a medium oil alkyd, a long oil alkyd, and a very long oil alkyd resin and/or coating comprise 50%, 45% to 50%, 60% to 70%, or 85% to 100% nonvolatile component, respectively.

In certain embodiments, a short oil alkyd coating may be selected as an industrial coating. In certain aspects, a short oil alkyd is synthesized from an oil, wherein the oil comprises castor, dehydrated castor, coconut, linseed, soybean, tall, or a combination thereof. In some aspects, the oil of a short oil alkyd comprises a saturated fatty acid. Examples of a saturated fatty acid include, but are not limited to, caproic (“hexanoic,” “6:0”); caprylic (“octanoic,” “8:0”); lauric (“dodecanoic,” “12:0”); or a combination thereof. In particular facets, a short oil alkyd coating comprises a solvent, wherein the solvent comprises an aromatic hydrocarbon, isobutanol, VMP naphtha, xylene, or a combination thereof. In other facets, the aromatic solvent comprises a high boiling aromatic solvent. In some aspects, a short oil alkyd is insoluble or poorly soluble in an aliphatic hydrocarbon. In further embodiments, a short oil alkyd coating undergoes film formation by baking.

In certain embodiments, a medium oil alkyd coating may be selected as a farm implement coating, a railway equipment coating, a maintenance coating, or a combination thereof. In certain aspects, a medium oil alkyd is synthesized from an oil, wherein the oil comprises linseed, safflower, soybean, sunflower, tall, or a combination thereof. In some aspects, the oil of a medium oil alkyd comprises a monounsaturated fatty acid (e.g., oleic acid). In particular facets, a medium oil alkyd coating comprises a solvent, wherein the solvent comprises an aliphatic hydrocarbon, an aromatic hydrocarbon, or a combination thereof.

In certain embodiments, a tall oil alkyd coating may be selected as an architectural coating, a maintenance coating, a primer, a topcoat, or a combination thereof. In certain aspects, a tall oil alkyd is synthesized from an oil, wherein the oil comprises linseed, safflower, soybean, sunflower, tall, or a combination thereof. In some aspects, the oil of a long oil alkyd comprises a polyunsaturated fatty acid. In particular facets, a tall oil alkyd coating comprises a solvent, wherein the solvent comprises an aliphatic hydrocarbon.

In certain embodiments, a very long oil alkyd coating may be selected as a latex architectural coating, a wood stain, or a combination thereof. In certain aspects, a very long oil alkyd is synthesized from an oil, wherein the oil comprises linseed, soybean, tall, or a combination thereof. In some aspects, the oil of a long oil alkyd comprises a polyunsaturated fatty acid. In particular facets, a very long oil alkyd coating comprises a solvent, wherein the solvent comprises an aliphatic hydrocarbon.

(ii) High Solid Alkyd Coatings

A high solid alkyd possesses a reduced viscosity, a lower average molecular weight, or a combination thereof. A high solid alkyd may be selected for embodiments wherein a reduced quantity liquid content (e.g., solvent) of a coating is desired. In some embodiments, a high solid alkyd coating comprises an enamel coating. In other aspects, a high solid long or very long oil alkyd coating comprises an architectural coating. In further aspects, a high solid medium oil alkyd coating comprises a transportation coating. In further aspects, a high solid short oil alkyd coating comprises an industrial coating. Additional, various chemical moieties may be incorporated in an alkyd to modify a property. Examples of such moieties include an acrylic, a benzoic acid, an epoxide, an isocyanate, a phenolic, a polyamide, a rosin, a silicon, a styrene (e.g., a paramethyl styrene), a vinyl toluene, or a combination thereof. In certain embodiments, a benzoic acid modified high solid alkyd coating comprises a coating for a tool. In other embodiments, a phenolic modified high solid alkyd coating comprises a primer. A silicone modified alkyd coating may be selected for improved weather resistance, heat resistance, or a combination thereof. In specific aspects, a silicone modified alkyd coating may comprise an additional binder capable of cross-linking with the silicone moiety (e.g., a melamine formaldehyde resin). In specific facets, a silicone modified alkyd coating may be selected as a coil coating, an architectural coating, a metal coating, an exterior coating, or a combination thereof. In certain facets, a high solid silicon-modified alkyd coating may substitute an oxygenated compound (e.g., a ketone, an ester) for an aromatic hydrocarbon liquid component. However, a high solid silicon-modified alkyd coating, to achieve cross-linking during film-formation, should comprise an additional binder capable of cross-linking. In further embodiments, a silicone modified high solid alkyd coating comprises a maintenance coating, a topcoat, or a combination thereof.

(iii) Uralkyd Coatings

An uralkyd binder (“uralkyd,” “urethane alkyd,” “urethane oil,” “urethane modified alkyd”) is an alkyd binder, with the modification that compound comprising plurality of diisocyanate moieties partly or fully replacing the dibasic acid (e.g., phthalic anhydride) in the synthesis reactions. Examples of an isocyanate comprising compounds include a 1,6-hexamethylene diisocyanate (“HDI”), a toluene diisocyanate (“TDI”), or a combination thereof. An uralkyd binder may be selected for embodiments wherein a superior abrasion resistance, superior resistance to hydrolysis, or a combination thereof, relative to an alkyd, is desired in a film. However, an uralkyd binder prepared using TDI often has greater viscosity in a coating, inferior color retention in a film, or a combination thereof, relative to an alkyd binder. Additionally, an uralkyd binder prepared using an aliphatic isocyanate generally possesses superior color retention to an uralkyd prepared from TDI. An uralkyd coating tends to undergo film formation faster than a comparable alkyd binder, due to a generally greater number of available conjugated double bonds, an increased T_{g} in an uralkyd binder prepared using an aromatic isocyanate, or a combination thereof. A film comprising an uralkyd binder tends to develop a yellow to brown color. An uralkyd binder is often used in preparation of an architectural coating such as a varnish, an automotive refinish coating, or a combination thereof. Examples of a surface where an uralkyd coating may be applied include a furniture surface, a wood surface, or a floor surface.

(iv) Water-Borne Alkyd Coatings

In general embodiments, an alkyd coating is a solvent-borne coating. However, an alkyd (e.g., a chemically modified alkyd) may be combined with a coupling solvent and water to produce a water-borne alkyd coating. Examples of a coupling solvent that may confer water reducibility to an alkyd resin includes ethylene glucol monobutyether, propylene glycol monoethylether, propylene glycol monopropylether, an alcohol whose carbon content is four carbon atoms (e.g., s-butanol), or a combination thereof. In certain embodiments, a water-borne long oil alkyd coating may be selected as a stain, an enamel, or a combination thereof. In other embodiments, a water-borne medium oil alkyd coating may be selected as an enamel, an industrial coating, or a combination thereof. In further facets, a water-borne medium oil alkyd coating may undergo film formation by air oxidation. In other embodiments, a water-borne short oil alkyd coating may be selected as an enamel, an industrial coating, or a combination thereof. In further facets, a water-borne short oil alkyd coating may undergo film formation by baking.

(3) Oleoresinous Binders

An oleoresinous binder is a type of binder prepared from heating a resin and an oil. Examples of a resin typically used in the preparation of an oleoresinous binder include resins obtained from a biological source (e.g., a wood resin, a bitumen resin); a fossil source (e.g., copal resin, a Kauri gum resin, a rosin resin, a shellac resin); a synthetic source (e.g., a rosin derivative resin, a phenolic resin, an epoxy resin); or a combination thereof. An example of an oil typically used in the preparation of an oleoresinous binder includes a vegetable oil, particularly an oil that comprises a polyunsaturated fatty acid such as tung, linseed, or a combination thereof. The type of resin and oil used can identify an oleoresinous binder such as a copal-tung oleoresinous binder, a rosin-linseed oleoresinous binder, etc. An oleoresinous binder generally is used in clear varnishes such as a lacquer, as well as in applications as a primer, an undercoat, a marine coating, or a combination thereof. In addition to the standards and analysis techniques previously described for an oil, standards for physical properties, chemical properties, and/or procedures for testing the purity/properties (e.g., glass transition temperature, molecular weight, color stability) of a hydrocarbon resin (e.g., a synthetic source resin) for use in an oleoresinous binder or other coating component are described, for example, in “ASTM Book of Standards, Volume 06.03, Paint—Pigments, Drying Oils, Polymers, Resins, Naval Stores, Cellulosic Esters, and Ink Vehicles,” E28-99, D6090-99, D6440-01, D6493-99, D6579-00, D6604-00, and D6605-00, 2002.

Similar to alkyd resins, oleoresinous binders can be categorized by oil length as a short oil or long oil oleoresinous binder, depending whether oil length is 1% to 67% or 67% to 99% oil, including all intermediate ranges and combinations thereof, respectively. Short oil oleoresinous binders generally dry fast and form relatively harder, less flexible films, and are used, for example, for floor varnishes. Long oil oleoresinous binders generally dry slower and form relatively more flexible films, and are used, for example, as an undercoat, exterior varnish, or combination thereof

(4) Fatty Acid Epoxy Esters

In certain facets, an epoxy coating may be cured by fatty acid oxidation rather than epoxide moiety or hydroxyl moiety cross-linking reactions. A fatty acid epoxide ester resin is an ester of an epoxide resin and a fatty acid, which can be used to produce an ambient cure coating that undergoes film formation by oxidative reactions as an oil-based coating. In certain embodiments, an epoxy resin may be selected with an epoxy equivalent weight of 800 to 1000, including all intermediate ranges and combinations thereof. Short, medium, and long oil epoxide ester resins comprise 30% to 50%, 50% to 70%, or 70% to 90% fatty acid esterification, including all intermediate ranges and combinations thereof, respectively, with similar, though sometimes superior, properties relative to an analogous alkyd. An epoxide ester resin generally is inferior in chemical resistance than a film produced by an epoxy and a curing agent comprising an amine. An epoxy ester resin may be selected as a substitute for an alkyd, a marine coating, an industrial maintenance coating, a floor topcoat, or a combination thereof.

b. Polyester Resins

A polyester resin (“polyester,” “oil-free alkyd”) is a polyester chemical, other than an alkyd resin, capable as use as a binder. A polyester resin is chemically very similar to an alkyd, though the oil content is about 0%. Consequently, a polyester-coating does not form cross-linking bonds by fatty acids oxidation during thermosetting film formation, but rather is combined with an additional binder to form a cross-linked film. The selection of a polyester and additional binder combination is generally determined by the polyester's crosslinkable moieties. For example, a hydroxy-terminated polyester is a polyester produced by an esterification reaction comprising a molar excess of a polyol, and may be crosslinked with a urethane, an amino resin, or a combination thereof. A hydroxy-terminated polyester's hydroxyl moiety may react with a urethane's isocyanate moiety such as at ambient conditions or low-bake conditions, while such a polyester generally undergoes film formation at baking temperatures with an amino resin. In another example, a “carboxylic acid-terminated polyester” is a polyester produced by an esterification reaction comprising an molar excess of a polycarboxylic acid, and may be crosslinked with a urethane, an amino resin, a 2-hydroxylakylamide, or a combination thereof.

In general embodiments, a polyester-coating possesses superior color retention, flexibility, hardness, weathering, or a combination thereof, relative to an alkyd-coating. In some embodiments, a polyester resin may be selected to produce a coating for a metal surface. Generally, a polyester-coating possesses a superior adhesion property on a metal surface than a thermosetting acrylic-coating. Often, a polyester-coating is a thermosetting coating, particularly in embodiments for use upon a metal surface. However, a polyester-coating generally comprises an ester linkage that is susceptible to hydrolysis, such as occurs in applications wherein such a polyester-coating contacts water.

A polyester resin is generally prepared by an acid catalyzed esterification of a polyacid (e.g., a polycarboxylic acid, an aromatic polyacid) and a polyalcohol. A “polyacid” (“polybasic acid”) is a chemical comprising more than one acid moiety. Typically, a polyacid used in the preparation of a polyester comprise two acidic moieties, such as, for example, an aromatic dibasic acid, an anhydride of an aromatic dibasic acid, an aliphatic dibasic acid, or a combination thereof. Usually, a polyester resin comprises a plurality of polycarboxylic acids and/or polyalcohols, and such a polyester resin is known herein as a “copolyester resin.” Examples of polycarboxylic acids commonly used to prepare a polyester resin includes adipic acid (“AA”); azelic acid (“AZA”); dimerized fatty acid; dodecanoic acid; hexahydrophthalic anhydride (“HHPA”); isophthalic acid (“IPA”); phthalic anhydride (“PA”); sebacid acid; terephthalic acid; trimellitic anhydride; or a combination thereof. Examples of a polyalcohol commonly used to prepare a polyester resin include 1,2-propanediol; 1,4-butanediol; 1,4-cyclohexanedimethanol (“CHDM”); 1,6-hexanediol (“HD”); diethylene glycol; ethylene glycol; glycerol; neopentyl glycol (“NPG”); pentaerythitol (“PE”); trimethylolpropane (“TMP”); or a combination thereof. In certain embodiments, a polyester may be selected that has been synthesized by an acid catalyzed esterification reaction between a plurality of polyalcohols comprising two hydroxy moieties (a “diol”), a polyalcohol comprising three hydroxy moieties (a “triol”), and a dibasic acid. An example of a diol includes 1,4-cyclohexanedimethanol; 1,6-hexanediol; neopentyl glycol; or a combination thereof. An example of a triol includes trimethylolpropane. An example of a polyol comprising four hydroxy moieties (a “tetraol”) includes pentaerythitol. In addition to the standards and analysis techniques previously described for an oil, an alkyd, a polyol, an acid anhydride standards for physical properties, chemical properties, and/or procedures for testing the purity/properties of an polyester are described, for example, in “ASTM Book of Standards, Volume 06.03, Paint—Pigments, Drying Oils, Polymers, Resins, Naval Stores, Cellulosic Esters, and Ink Vehicles,” D2690-98 and D3733-93, 2002.

The selection of a polyacid and/or a polyalcohol often affects a property of the polyester resin, such as the resistance of the polyester resin to hydrolysis, and similarly the water resistance of a coating and/or film comprising such a polyester resin. In embodiments wherein a polyester-coating is desired with a superior water resistance property relative to other types of polyester-coatings, it is contemplated for some embodiments that the coating comprises a polyester prepared with a polyol that is more difficult to esterify, and thus generally more difficult to hydrolyze. Examples of such polyols include neopentyl glycol, trimethylolpropane1,4-cyclohexanedimethanol, or a combination thereof.

In general embodiments, a polyester-coating is a solvent-borne coating. However, a polyester can be suitable for a water-borne coating. A water-borne polyester-coating generally comprises a polyester resin, wherein the acid number of the polyester resin is 40 to 60 including all intermediate ranges and combinations thereof, and wherein the acid moieties have been neutralized by an amine, and wherein the coating comprises liquid component that comprises a co-solvent. An additional water-borne binder (e.g., an an amino resin) may be used to produce thermosetting film formation. In specific aspects, a water-borne polyester-coating produces a film of excellent hardness, gloss, flexibility, or a combination thereof.

In alternative embodiments, a polyester temporary coating (e.g., a non-film forming coating) may be produced, for example, by selection of a polyester that comprises fewer or no crosslinkable moieties, selection of an additional binder that comprises fewer or no crosslinkable moieties, reducing the concentration of the polyester or additional binder, or a combination thereof.

c. Modified Cellulose Binders

In some embodiments, a chemically modified cellulose molecule (“modified cellulose,” “cellulosic”) may be used as a coating component (e.g., a binder). Cellulose is a polymer of anhydroglucose monomers that is insoluble in water and organic solvents. Various chemically modified forms of cellulose with enhanced solubility have been used as a coating component. Examples of chemically modified cellulose (“modified cellulose,” “cellulosic”) include a cellulose ester, a nitrocellulose, or a combination thereof. Examples of a cellulose ester include cellulose acetate (“CA”), cellulose butyrate, cellulose acetate butyrate (“CAB”), cellulose acetate propionate (“CAP”), a hydroxy ethyl cellulose, a carboxy methyl cellulose, cellulose acetobutyrate, ethyl cellulose, or a combination thereof. A cellulose ester coating typically produces films with excellent flame resistance, toughness, clarity, or a combination thereof. In certain embodiments, a cellulose ester coating is selected as a topcoat, a clear coating, a lacquer, or a combination thereof. A cellulose ester is often selected for embodiments wherein the coating comprises an automotive coating, a furniture coating, a wood surface coating, cable coating, or a combination thereof. A cellulose ester coating may be a thermoplastic coating, a thermosetting coating, or a combination thereof.

A cellulose ester may be selected by the properties associated with the degree and/or type of esterification. Typically, solubility in a liquid component and/or combinability with an additional binder is increased by partial esterification of an anhydroglucose's hydroxy moieties. For example, for a cellulose acetate butyrate, properties such as compatibility, diluent tolerance, flexibility (e.g., lower T_{g}), moisture resistance, solubility, or a combination thereof, increases with greater butyrate esterification. However, decreased hydroxyl content alters properties in a cellulose ester. For example, a cellulose acetate butyrate comprising a hydroxy content of 1% or below has limited solubility in many solvents, while a hydroxy content of 5% or greater allows solubility in many alcohols, and the increased number of hydroxy moieties allows a greater degree of cross-linking reactions with binders such as, for example, an amino binder, an an acrylic binder, urethane binder, or a combination thereof. A cellulose acetate butyrate acrylic-coating may be selected as lacquers, an automotive coating, a coating comprising a metallic pigment (e.g., aluminum), or a combination thereof. A cellulose acetate butyrate acrylic-coating may comprise a liquid component that comprises greater amounts of an aromatic hydrocarbon solvent with the selection of a CAB with greater butyrate ester content. Though not a cellulosic, sucrose esters may be similarly used as cellulose ester, particularly CAB.

In some embodiments, in a cellulose ester comprising an acetyl ester (e.g., comprises cellulose acetate, cellulose acetate butyrate, cellulose acetate propionate), the acetyl content will range from 0.1% to 40.5% acetate, including all intermediate ranges and combinations thereof. In certain aspects, the acetyl content of a cellulose acetate, a cellulose acetate butyrate, or a cellulose acetate propionate will range from 39.0% to 40.5%, 1.0% to 30.0%, or 0.3% to 3.0%, respectively, including all intermediate ranges and combinations thereof, respectively. In many aspects, in a cellulose ester comprising a butyryl ester (e.g., cellulose acetate butyrate), the butyryl content will range from 15.0% to 55.0% butyryl, including all intermediate ranges and combinations thereof. In other aspects, in a cellulose ester comprising a propionyl ester (e.g., cellulose acetate propionate), the propionyl content will range from 40.0% to 47.0% propionyl, including all intermediate ranges and combinations thereof. In other embodiments, the hydroxyl content of a cellulose acetate, a cellulose acetate butyrate, or a cellulose acetate propionate will range from 0% to 5.0%, including all intermediate ranges and combinations thereof.

A nitrocellulose (“cellulose nitrate”) resin comprises a cellulose molecule wherein a hydroxyl moiety has been nitrated. A nitrocellulose for use in a coating typically comprises an average of 2.15 to 2.25 nitrates per anhydroglucose monomer, and is soluble in an ester, a ketone, or a combination thereof. Additionally, nitrocellulose is soluble in a combination of a ketone, an ester, and an alcohol and/or hydrocarbon. A nitrocellulose may be selected as a lacquer, an automotive primer, automotive topcoat, a wood topcoat, or a combination thereof. Nitrocellulose coatings are typically a thermoplastic coating.

Standard procedures for determining physical and/or chemical properties (e.g., acetyl content, ash, apparent acetyl content, butyryl content, carbohydrate content, carboxyl content, color and haze, combined acetyl, free acidity, heat stability, hydroxyl content, intrinsic viscosity, solution viscosity, moisture content, propionyl content, sulfur content, sulfate content, metal content), of a cellulose and/or a modified cellulose (e.g., cellulose acetate, cellulose acetate propionate, cellulose acetate butyrate, methylcellulose, sodium carboxymethylcellulose, ethylcellulose, hydroxypropyl methylcellulose, hydroxyethylcellulose, hydroxypropylcellulose) have been described, for example, in “ASTM Book of Standards, Volume 06.03, Paint—Pigments, Drying Oils, Polymers, Resins, Naval Stores, Cellulosic Esters, and Ink Vehicles,” D1695-96 D817-96, D871-96, D1347-72, D1439-97, D914-00, D2363-79, D2364-01, D5400-93, D1343-95, D1795-96, D2929-89, D3971-89, D4085-93, D1926-00, D4794-94, D3876-96, D3516-89, D5897-96, D5896-96, D6188-97, D1348-94, and D1696-95, 2002. Specific procedures for determining purity/properties of a nitrocellulose (e.g., nitrogen content) have been described, for example, in “ASTM Book of Standards, Volume 06.03, Paint—Pigments, Drying Oils, Polymers, Resins, Naval Stores, Cellulosic Esters, and Ink Vehicles,” D301-95 and D4795-94, 2002.

In alternative embodiments, a modified cellulose temporary coating (e.g., a non-film forming coating) may be produced, for example, by selection of a modified cellulose that comprises fewer or no crosslinkable moieties, selection of an additional binder that comprises fewer or no crosslinkable moieties, reducing the concentration of the modified cellulose or additional binder, or a combination thereof.

d. Polyamide and Amidoamine Binders

A polyamide (“fatty nitrogen compound,” “fatty nitrogen product”) is a reaction product of a polyamine and a dimerized and/or trimerized fatty acid. In typical embodiments, a polyamide is an oligomer. An amide resin comprises a terminal amine moiety capable of cross-linking with an epoxy moiety, and it is contemplated for some embodiments that a polyamide binder is combined with an epoxide binder. In other aspects, a polyamide may be considered an additive (e.g., a curing agent, a hardening agent, a coreactant) of an epoxide coating. A polyamine-epoxy coating may be used as an industrial coating (e.g., an industrial maintenance coating), a marine coating, or a combination thereof. A polyamide-epoxide coating may be applied to a surface such as, for example, wood, masonry, metal (e.g., steel), or a combination thereof. However, in some embodiments, a surface is thoroughly cleaned prior to application to promote adhesion. Such surface preparation in the art may be used, and include, for example, removal of rust, degraded film, grease, etc. A polyamide-epoxy coating typically is a solvent-borne coating. Examples of solvents for a polyamide include an alcohol, an aromatic hydrocarbon, a glycol ether, a ketone, or a combination thereof. In certain embodiments, a polyamide-epoxy coating may comprise a two-pack coating, wherein coating component(s) comprising the polyamide resin are stored in one container, and coating components comprising the epoxy resin are stored in a second container. Such a two-pack coating is admixed immediately before application, as the stoichiometric mix ratio of resin is formulated to promote a rapid cure. However, in other embodiments, a polyamide-epoxy coating may be a single container coating. Such a solvent-borne polyamine-epoxy coating may be formulated for a storage life of a year or more. An aluminum and or stainless steel container is suitable, though a carbon steel container may alter coating and/or film color. However, such a coating typically undergoes film formation in stages, wherein the liquid component is physically lost by evaporation while thermosetting produces a physically durable film in about 8 to 10 hours, a chemically resistant film in three to four days, and final cross-linking completed in about three weeks. In some embodiments, a polyamine-epoxy coating may undergo chalking upon exterior weathering.

Though a polyamide is prepared from a fatty acid, it is not classified as an oil-based binder herein due to the chemistry of film formation for polyamide binder. The dimerized (“dibasic”) or trimerized fatty acid generally comprises a polyunsaturated fatty acid, a monounsaturated fatty acid, or a combination thereof. In certain aspects, the fatty acid is a linseed oil fatty acid, soybean oil fatty acid, tall oil fatty acid, or a combination thereof. In specific facets, the fatty acid is an 18-carbon fatty acid. However, to reduce the volatile organic compounds of solvent-borne coating, a polyamide binder may be partly or fully substituted, such as 0% to 100% substitution, including all intermediate ranges and combinations thereof, with an amidoamine binder. An amidomine binder differs from a polyamide binder by the use of a fatty acid rather than a dimerized fatty acid in the synthesis of the resin. The selection of the polyamine in the preparation of a polyamide can affect the properties of the polyamide. The polyamine may be linear (e.g., diethylenetriamine), branched or cyclic (e.g., aminoethylpiperazine). Standards for physical properties, chemical properties, and/or procedures for testing the purity/properties (e.g., amine value) of a polyamide and/or an amidoamine are described, for example, in “ASTM Book of Standards, Volume 06.03, Paint—Pigments, Drying Oils, Polymers, Resins, Naval Stores, Cellulosic Esters, and Ink Vehicles,” D2071-87, D2073-92, D2082-92, D2072-92, D2074-92, D2075-92, D2076-92, D2077-92, D2078-86, D2079-92, D2080-92, D2081-92, and D2083-92, 2002.

In general embodiments, a polyamine comprises a polyethylene amine. A polyamide produced from diethylenetriamine can be prepared to comprise a varying amount, typically 35% to 85%, including all intermediate ranges and combinations thereof, of an imidazoline moiety. In other embodiments, the amount of amine moiety capable of cross-linking with an epoxy moiety may vary from 100 to 400 amine value, including all intermediate ranges and combinations thereof. However, the amine value is converted into units known as “active hydrogen equivalent weight,” which varies from 550 to 140, including all intermediate ranges and combinations thereof, for comparison to the epoxy resins epoxide equivalent weight for determining the stoichiometric mix ratio of a polyamide-epoxy combination. The stoichiometric mix ratio affects coating and film properties. As the polyamide to epoxy stoichiometric mix ratio increases from a ratio of less than one to a ratio of greater than one, properties such as excellent impact resistance, excellent chemical resistance, or a combination thereof, decrease while film flexibility increases. Examples of polyamide to epoxy stoichiometric mix ratio include 2:1 to 1:2, including all intermediate ranges and combinations thereof.

In alternative embodiments, a polyamide and/or amidoamine temporary coating (e.g., a non-film forming coating) may be produced, for example, by selection of a polyamide and/or amidoamine that comprises fewer or no crosslinkable moieties, selection of an additional binder that comprises fewer or no crosslinkable moieties, reducing the concentration of the polyamide and/or amidoamine or additional binder, selection of a stoichiometric ratio that is less suitable for crosslinking reactions, or a combination thereof.

e. Amino Resins

An amino resin (“amino binder,” “aminoplast,” “nitrogen resin”) is a reaction product of formaldehyde, an alcohol and a nitrogen compound such as, for example, urea, melamine (“1:3:5 triamino triazine”), benzoguanamine, glucoluril, or a combination thereof. An amino resin may be used to a thermosetting coating. An amino resin comprises an alkoxymethyl moiety capable of cross-linking with a hydroxyl moiety of an additional binder such as an acrylic binder, an alkyd resin, a polyester binder, or a combination thereof, and in certain embodiments an amino resin is combined with a binder that comprises a hydroxyl moiety in a coating. In some aspects wherein the coating comprises an amino resin and an alkyd resin, the amino:alkyd resin ratio is 1:1 to 1:5, including all intermediate ranges and combinations thereof. An amino resin coating typically is a solvent-borne coating. Examples of solvents for an amino resin include an alcohol (e.g., butanol, isobutanol, methanol, isopropanol), a ketone, hydroxyl functional glycol ether, or a combination thereof. Additionally, an amino resin generally possesses limited solubility in a hydrocarbon (e.g., xylene), which may be added to a solvent-borne coating's liquid component. In certain aspects, an amino resin coating may be a water-borne coating, wherein water is a solvent for an amino resin comprising a plurality of methylol moieties. In other embodiments, a water-borne amino resin coating may comprise a water-reducible coating, particularly wherein the liquid component comprises a glycol ether, an alcohol, or a combination thereof. In certain embodiments, an amino coating comprises an acid catalyst.

An amino resin coating generally is cured by baking at a temperature of 82° C. and 204° C., including all intermediate ranges and combinations thereof. Baking generally promotes reactions between amino resins, though it does improve the reaction rate between an amino resin and an additional binder. In some embodiments wherein the coating comprises an additional binder, the additional resin comprises less hydroxyl moieties and/or the amino resin is polar amino resin (e.g., a conventional amino resin) when cured by baking than embodiments wherein an acid catalyst is used. An amino resin coating undergoes rapid film formation, typically lasting 30 seconds and 30 minutes, wherein a higher temperature and/or acid catalyst shortens film formation time. An amino resin prepared from urea is generally undergoes film formation faster than an amino resin prepared from melamine. However, an amino resin coating generally produces an alcohol (e.g., methanol, butanol) and formaldehyde during film formation as byproducts.

An amino resin for use in a coating may be classified by content of a liquid component (e.g., a solvent) as a high solids amino resin or a conventional amino resin. The liquid component is generally used to reduce the viscosity of the resin for coating preparation. A high solids amino resin comprises 80% to 100%, by weight, an amino resin, with the balance a liquid component. A high solids amino resin is are relatively less polar, less polymeric, lower in viscosity, or a combination thereof, relative to a conventional amino resin. The lower viscosity allows the use of little or no liquid component. Additionally, a high solids amino resin may be water-soluble and/or water reducible. A conventional amino resin comprises less than 80% amino resin, by weight, with the balance a liquid component. Properties of a high solids or conventional amino resin selected for use in a coating such as the amount of amino resin and liquid component, the amount of unreacted formaldehyde in the resin preparation, the viscosity of the resin, the ability of the resin to accept additional liquid component as a solvent, can be empirically determined (see, for example, “ASTM Book of Standards, Volume 06.03, Paint—Pigments, Drying Oils, Polymers, Resins, Naval Stores, Cellulosic Esters, and Ink Vehicles,” D4277-83, D1545-98, D1979-97, and D1198-93, 2002; and “ASTM Book of Standards, Volume 06.01, Paint—Tests for Chemical, Physical, and Optical Properties; Appearance,” D2369-01e1, 2002).

In embodiments wherein an amino resin coating comprises an amino resin prepared from urea, the coating may be used as wood coating (e.g., furniture coating), an industrial coating (e.g., an appliance coating), an automotive primer, a clear coating, or a combination thereof. However, an amino resin film, wherein the resin was prepared from urea, generally produces a film with poor resistance to moisture, and is contemplated as an internal coating and/or as part of a multicoat system. In certain embodiments, an amino resin prepared from melamine, generally produces films with good resistance to moisture, temperature, UV irradiation, or a combination thereof. A melamine-based amino coating may be applied to a metal surface. In specific aspects, such a melamine amino resin coating may be an automotive coating, a coil coating, a metal container coating, or a combination thereof. In embodiments wherein an amino resin coating comprises an amino resin prepared from benzoguanamine, the film produced generally possesses poor weathering resistance, good corrosion resistance, water resistance, detergent resistance, flexibility, hardness, or a combination thereof. A benzoguanamine amino resin may be used as an industrial coating, particularly for indoor applications (e.g., an appliance coating). In embodiments wherein an amino resin coating comprises an amino resin prepared from, glycoluril, a higher baking temperature and/or acid catalyst may be used during film formation, but less byproducts may be released. A glycoluril-based amino-coating typically produces a film with excellent corrosion resistance, humidity resistance, or a combination thereof. A glycoluril-based amino-coating may be selected as a metal coating.

In alternative embodiments, an amino resin temporary coating (e.g., a non-film forming coating) may be produced, for example, by selection of an amino resin that that comprises fewer or no crosslinkable moieties, selection of an additional binder that comprises fewer or no crosslinkable moieties, reducing the concentration of the amino resin and/or additional binder, selection of a binder ratio that is less suitable for crosslinking reactions, using a bake cured amino resin coating at temperatures less than is needed for curing (e.g., ambient conditions) or a combination thereof.

f. Urethane Binders

A urethane binder (“polyurethane binder,” “urethane,” “polyurethane”) is a binder prepared from compounds that comprise an isocyanate moiety. The urethane binder's urethane moiety can form intermolecular hydrogen bonds between urethane binder polymers, and these non-covalent bonds confer useful properties in a coating or film comprising a urethane binder. The hydrogen bonds can be broken by mechanical stress, but will reform, thereby conferring a property of abrasion resistance. Additionally, a urethane binder can form some hydrogen bonds with water, conferring a plasticizing property to the coating. In certain embodiments, a urethane binder comprises an isocyanate moiety. The isocyanate moiety is reactive (e.g., crosslinkable) with a moiety comprising a chemically reactive hydrogen. Examples of a chemically reactive hydrogen moiety include a hydroxyl moiety, an amine moiety, or a combination thereof. Examples of an additional binder include a polyol, an amine, an epoxide, silicone, vinyl, phenolic, or a combination thereof. In certain embodiments, a urethane coating is a thermosetting coating. In specific aspects, a urethane coating comprises a catalyst (e.g., dibutyltin dilaurate, stannous octoate, zinc octoate). In specific facets, the coating comprises 10 to 100 parts per million catalyst, including all intermediate ranges and combinations thereof. In some embodiments, such a coating will undergo film formation at ambient conditions or slightly greater temperatures. A binder comprising an isocyanate moiety is often selected to produce a coating with durability in an external environment. A urethane coating typically possesses good flexibility, toughness, abrasion resistance, chemical resistance, water resistance, or a combination thereof. An aliphatic urethane coating may be selected for the additional property of good lightfastness.

In general embodiments, a urethane binder may be selected based on the materials used in its preparation, which typically affect the urethane binder's properties. An example of a urethane binder includes an aromatic isocyanate urethane binder, an aliphatic isocyanate urethane binder, or a combination thereof. Aliphatic isocyanate urethane binders are often selected for embodiments wherein a superior exterior durability, color stability, good lightfastness, or a combination thereof relative to an aromatic isocyanate binder is desired. Examples of an aliphatic isocyanate urethane binder includes a hydrogenated bis(4-isocyanatophenyl)methane (“4,4′dicyclohexylmethane diisocyanate,” “HMDI”), HDI, a combination of 2,2,4-trimethyl hexamethylene diisocyanate and 2,4,4-trimethyl hexamethylene diisocyanate (“TMHDI”), 1,4-cyclohexane diisocyanate (“CHDI”), isophorone diisocyanate (“3-isocyanatomethyl-3,5,5-trimethylcyclohexyl isocyanate,” “IPDI”), or a combination thereof. In certain aspects, a HDI derived binder is prepared from excess HDI reacted with water, known as “HDI biuret.” In certain aspects, a HDI derived binder may be prepared from a 1,6-hexamethylene diisocyanate isocyanurate, wherein such a HDI derived binder produces a coating with generally superior heat resistance and/or exterior durability is desired relative to other HDI derived binders. Standards for physical properties, chemical properties, and/or procedures for testing the purity/properties of urethane precursor components (e.g., toluene) and urethane resins (e.g., isocyanate moieties) for use in a coating are described, for example in “ASTM Book of Standards, Volume 06.04, Paint—Solvents; Aromatic Hydrocarbons,” D5606-01, 2002; and “ASTM Book of Standards, Volume 06.03, Paint—Pigments, Drying Oils, Polymers, Resins, Naval Stores, Cellulosic Esters, and Ink Vehicles,” D3432-89 and D2572-97, 2002.

In certain embodiments, a urethane coating comprises a urethane binder capable of a self-crosslinking reaction. An example is a moisture-cure urethane, which comprises an isocyanate moiety. Contact between an isocyanate moiety and a water molecule produces an amine moiety capable of bonding with an isocyanate moiety of another urethane binder molecule in a linear polymerization reaction. In certain aspects, a moisture cure urethane coating is baked at 100° C. to 140° C., including all intermediate ranges and combinations thereof, to promote crosslinking reactions between the linear polymers. In certain embodiments, a moisture-cure urethane coating is a solvent-borne coating. In specific aspects, a moisture-cure urethane coating comprises a dehydrator. In general aspects, moisture-cure urethane coating typically is a one-pack coating, prepared for storage of the coating in anhydrous conditions.

In certain embodiments, a urethane coating comprises a blocked isocyanate urethane binder, wherein the isocyanate moiety has been chemically modified by a hydrogen donor to be inert until contacted with a baking temperature. Such a blocked isocyanate urethane coating typically is a one-pack coating, as it is designed for stability at ambient conditions. Additionally, a blocked isocyanate urethane coating may be a powder coating.

In certain embodiments, a urethane coating comprises an additional binder. In certain embodiments, a urethane may be combined with a binder such as an amine, an epoxide, silicone, vinyl, phenolic, a polyol, or a combination thereof, wherein the binder comprises a reactive hydrogen moiety. In specific embodiments, selection of a second binder to crosslink with the urethane binder affects coating and/or film properties. In certain aspects, a coating comprising a urethane and an epoxide, vinyl, phenolic, or a combination thereof produces a film with good chemical resistance. In other aspects, a coating comprising a urethane and a silicone produces a coating with good thermal resistance. In some aspects, a coating comprises a urethane and a polyol. A primary hydroxyl moiety, secondary hydroxyl moiety, and tertiary hydroxyl moiety of a polyol are respectively the fastest, moderate, and slowest to react with a urethane. Steric hindrance from a neighboring moiety may slow the reaction with a hydroxyl moiety. In an additional example, use of a polyol may increase flexibility of a urethane coating. Often, a selected polyol has a molecular weight from 200 Da to 3000 Da, including all intermediate ranges and combinations thereof. Generally, a lower molecular weight polyol increases the hardness property, lowers the flexibility property, or a combination thereof, of a urethane polyol film. Examples of a polyol include a glycol, a triol (e.g., 1,4-butane-diol, diethylene glycol, trimethylolpropane), a tetraol, a polyester polyol, a polyether polyol, an acrylic polyol, a polylactone polyol, or a combination thereof. Examples of a polyether polyol include a poly (propylene oxide) homopolymer polyol, a poly (propylene oxide) and ethylene oxide copolymer polyol, or a combination thereof.

In certain embodiments, a urethane binder comprises a thermoplastic urethane binder. Typically, a thermoplastic urethane binder is from 40 kDa to 100 kDa, including all intermediate ranges and combinations thereof. In particular aspects, a thermoplastic urethane binder comprises little or no isocyanate moieties. In general aspects, a thermoplastic urethane coating is a solvent borne coating. In specific facets, a thermoplastic urethane coating is a lacquer, a high gloss coating, or a combination thereof.

In certain embodiments, a urethane binder is a urethane acrylate (“acrylated urethane”) binder. A urethane acrylate binder generally comprises an acrylate moiety at an end of the polymeric binder. The acrylate moiety is typically part of an acrylate monomer, wherein the monomer comprises a hydroxyl moiety (e.g., a 2-hydroxy-ethyl acrylate). A urethane acrylate coating generally comprises another binder for crosslinking reactions. Examples of a suitable binder include a triacrylate (e.g., teimethylolpropane). A urethane acrylate coating generally also comprises a viscosifier, wherein the viscosifier reduces viscosity. Examples of such a viscosifer include an acrylate monomer, a N-vinyl pyrrolidone, or a combination thereof. A urethane acrylate coating is cured by irradiation. Examples of irradiation include UV light, electron beam, or a combination thereof. In embodiments wherein UV light is a curing agent, a urethane acrylate coating typically comprises a photoinitiator. Examples of a suitable initiator include 2,2,-diethoxyacetophenone, a combination of benzophenone and an amine synergist, or a combination thereof. In specific facets, a urethane acrylate coating is applied to a plastic surface. In other facets, a urethane acrylate coating floor coating, an electronic circuit board coating, or a combination thereof.

In alternative embodiments, a urethane temporary coating (e.g., a non-film forming coating) may be produced, for example, by selection of a urethane resin that that comprises fewer or no crosslinkable moieties, selection of an additional binder that comprises fewer or no crosslinkable moieties, reducing the concentration of the urethane resin and/or additional binder, using a bake cured urethane resin coating at temperatures less than is needed for curing (e.g., ambient conditions), selection of a size range for a thermoplastic urethane resin coating that is less suitable for film formation (e.g., 1 kDa to 40 kDa), or a combination thereof.

(1) Water-Borne Urethanes

The previous discussion of urethane coatings is focused on solvent-borne urethane coatings. A water-borne urethane coating typically comprises a water-dispersible urethane binder such as a cationic modified urethane binder and/or anionic modified urethane binder. A cationic modified urethane binder is a urethane binder chemically modified by a diol comprising an amine, such as, for example, diethanolamine, methyl diethanolamine, N,N-bis(hydroxyethyl)-α-aminopyridine, lysine, N-hydroxyethylpiperidine, or a combination thereof. An anionic modified urethane binder is a urethane binder chemically modified by a diol comprising a carboxylic acid such as dimethylolpropionic acid (2,2-bis(hydroxymethyl)propionic acid), dihydroxybenzoic acid, and/or a sulfonic acid (e.g., 2-hydroxymethyl-3-hydroxy-propanesulfonic acid), or a combination thereof.

(2) Urethane Powder Coatings

A urethane powder coating refers to a polyester and/or acrylic coating, wherein the binder has been modified to comprise a urethane moiety. Such a coating is typically a thermosetting, bake cured coating, an industrial coating (e.g., an appliance coating), or a combination thereof.

g. Phenolic Resins

A phenolic resin (“phenolic binder,” “phenolic”) is reaction product of a phenolic compound and an aldehyde. A type of aldehyde is formaldehyde, and such a phenolic resin is known as a “phenolic formaldehyde resin” (“PF resin”). The properties of a phenolic resin are affected by the phenolic compound and reaction conditions used during synthesis. A resole resin (“resole phenolic”) is prepared by a reaction of a molar excess of a phenolic compound with formaldehyde under alkaline conditions. A novolac resin (“novolac phenolic”) is prepared by a reaction of a molar excess of formaldehyde with a phenolic compound under acidic conditions. Examples of phenolic compounds used in preparing a phenolic resin include phenol; orthocresol (“o-cresol”); metacresol, paracresol (“p-cresol”); a xylenol (e.g., 4-xylenol); bisphenol-A [“2,2-bis(4-hydroxylphenyl)propane”; “diphenylol propane”); p-phenylphenol; p-tert-butylphenol; p-tert-amylphenol; p-tert-octyl phenol; p-nonylphenol; or a combination thereof. Standards for physical properties, chemical properties, and/or procedures for testing the purity/properties of various compounds used in phenolic resins (e.g., bisphenol A, a phenol, a cresol, formaldehyde) for use in a coating are described, for example in “ASTM Book of Standards, Volume 06.04, Paint—Solvents; Aromatic Hydrocarbons,” D6143-97, D3852-99, D4789-94, D2194-02, D2087-97, D2378-02, D2379-99, D2380-99, D1631-99, D6142-97, D4493-94, D4297-99, and D4961-99, 2002. Standards for physical properties, chemical properties, and/or procedures for testing the purity/properties of phenolic resins for use in a coating are described, for example in “ASTM Book of Standards, Volume 06.03, Paint—Pigments, Drying Oils, Polymers, Resins, Naval Stores, Cellulosic Esters, and Ink Vehicles,” D1312-93, D4639-86, D4706-93, D4613-86 and D4640-86, 2002.

In alternative embodiments, a phenolic resin temporary coating (e.g., a non-film forming coating) may be produced, for example, by selection of a phenolic resin that comprises fewer or no crosslinkable moieties, selection of an additional binder that comprises fewer or no crosslinkable moieties, reducing the concentration of the phenolic resin and/or additional binder, using a bake cured phenolic resin coating at temperatures less than is needed for curing (e.g., ambient conditions), or a combination thereof.

(1) Resole

A resole resin is the more commonly used PF resin. A solvent-borne phenolic formaldehyde coating typically comprises an alcohol, an ester, a glycol ether, a ketone, or a combination thereof, as a PF solvent. However, a phenolic resin prepared from phenolic compound comprising an alkyd moiety, such as, for example, p-tert-butylphenol p-tert-amylphenol p-tert-octyl phenol, or a combination thereof, typically has solubility in an aromatic compound and/or able to tolerate an aliphatic diluent. Often, a phenolic-resin coating comprises an additional binder such as an alkyd resin, an amino resin, a blown oil, an epoxy resin, a polyamide, a polyvinyl resin [e.g., poly(vinyl butyral)], or a combination thereof. An example of a phenolic-resin coating includes a varnish, an industrial coating, or a combination thereof. A phenolic resin-coating may be selected for embodiments wherein a film possessing solvent resistance, corrosion resistant, of a combination thereof, is desired. Examples of surfaces wherein such properties are often desirable include a surface of a metallic container (e.g., a can, a pipeline, a drum, a tank), a coil coating, or a combination thereof. In specific aspects, a phenolic coating produces a film 0.2 to 1.0 mil thick, including all intermediate ranges and combinations thereof. In specific aspects, coating comprising a phenolic-binder and additional binder undergoes thermosetting cross-linking reactions between the binders during film formation. In certain embodiments, a phenolic-resin coating undergoes cure by baking, such as, for example, 135° C. to 204° C., including all intermediate ranges and combinations thereof. In specific aspects, a baking cure time is one minute to four hours, with shorter cure times at high temperatures. A phenolic-resin film generally possesses excellent hardness property (e.g., glass-like), excellent resistance to solvents, water, acids, salt, electricity, heat resistance, as well as thermal resistance up to 370° C. for a period of minutes.

However, a phenolic-resin film is poorly resistant to alkali unless made from a coating that also comprised an epoxy binder. In certain embodiments, a phenolic-epoxy coating comprises a binder ratio of 15:85 to 50:50 phenolic binder:epoxy binder, including all intermediate ranges and combinations thereof. In certain aspects, a phenolic-epoxy coating possesses superior flexibility, toughness, or a combination thereof relative to a phenolic coating. In specific facets, a phenolic-epoxy coating is cured at 200° C. for 10 to 12 minutes.

In other aspects, a phenolic coating comprises a blown oil, an alkyd, or a combination thereof. In some aspects, such a coating comprises a phenolic resin prepared fromp-tert-butylphenol p-tert-amylphenol p-tert-octyl phenol, or a combination thereof. In specific aspects, such a coating is applied to electrical coil, electrical equipment, or a combination thereof

(2) Novolak

In other aspects, wherein a film is desired, a novolak coating may be used. However, a novolak resin is generally a non-film forming resin. In some specific aspects the coating comprise an epoxy resin. It is also contemplated in some facets that the coating comprise a basic catalyst. A film produced from such a novolak-epoxy coating typically possesses good resistance to chemicals, water, heat, or a combination thereof. In specific facets, a novolak-epoxy coating may be a high solids coating, a powder coating, a pipeline coating, or a combination thereof.

A novolak resin prepared from phenolic compound comprising an alkyd moiety such as p-tert-butylphenol p-tert-amylphenol p-tert-octyl phenol, or a combination thereof, typically has solubility in an oil. Additionally, a PF resin may be modified by reaction with an oil to produce an oil modified PF resin, which is also oil soluble. An alkyd phenol-formaldehyde resin, an oil modified phenol-formaldehyde resin, is generally a non-film forming resin. A coating capable of producing a film may be formulated by combining such a resin with a drying oil, an alkyd, or a combination thereof. In specific aspects, an alkyd phenol-formaldehyde resin, an oil modified phenol-formaldehyde resin undergoes cross-linking with an oil and/or an alkyd. Such a coating may further comprise a liquid component (e.g., a solvent), a drier, a UV absorber, an anti-skinning agent, or a combination thereof. In certain facets, such a coating undergoes film formation under ambient conditions or by baking. In particular aspects, such a coating comprises a varnish, a wood coating, or a combination thereof. In specific facets, such a coating comprises a pigment.

h. Epoxy Resins

An epoxy resin (“epoxy binder,” “epoxy”) is a compound comprising an epoxide (“oxirane”) moiety. An epoxide resin may be used in a thermosetting coating, thermoplastic coating, or a combination thereof. An epoxide coating typically is a solvent borne coating, though examples of a water-borne and powder epoxy coating are described herein. An epoxide coating generally possesses excellent properties of adhesion, corrosion resistance, chemical resistance, or a combination thereof. An epoxide coating may be selected for various surfaces, particularly a metal surface.

An epoxide resin (e.g., a bisphenol A epoxy resin) generally comprises one or two epoxide moieties per resin molecule. An epoxide resin may additionally comprise a monomer, oligomer, or polymer of repeating chemical units, each generally lacking an epoxide moiety, but comprising a hydroxy moiety. The number of monomer(s) present is expressed “n” value, wherein an average increase of one monomer per epoxide resin molecule increase the n value by one. The chemical and/or physical properties of an epoxide resin are affected by the n value. For example, as the n value increases, the chemical reactions selected for film formation in a thermosetting coating may become more dominated by reactions with the increasing numbers of hydroxyl moieties, and less dominated by the epoxide moieties. Often, an epoxide resin is classified by an epoxide equivalent weight, which is the grams of resin required to provide 1 M epoxide moiety equivalent. In certain embodiments, the epoxide equivalent weight is 182 to 3050, including all intermediate ranges and combinations thereof. Additionally, an epoxide resin may be used in a thermoplastic coating, particularly wherein the n value is greater than 25. In certain embodiments, an epoxide resin may possess an n value of 0 to 250, including all intermediate ranges and combinations thereof. Standards for physical properties, chemical properties, and/or procedures for testing the purity/properties of epoxy resins (e.g., epoxy moiety content) for use in a coating are described, for example in “ASTM Book of Standards, Volume 06.03, Paint—Pigments, Drying Oils, Polymers, Resins, Naval Stores, Cellulosic Esters, and Ink Vehicles,” D4142-89, D1652-97, D1726-90, D1847-93, and D4301-84, 2002.

An epoxide moiety is chemically reactive with a variety of other moieties, such as, for example, an amine, a carboxyl, a hydroxyl or a phenol. An epoxide coating may comprise an additional binder capable of undergoing a cross-linking reaction with the epoxide during film formation. Various such additional binders in the art are often referred to as a “curing agent” or “hardener.” The selection of a curing agent and/or an epoxide can affect whether the coating undergoes film formation at ambient conditions or by baking.

In alternative embodiments, an epoxide resin temporary coating (e.g., a non-film forming coating) may be produced, for example, by selection of an epoxide resin that comprises fewer or no crosslinkable moieties, selection of an additional binder that comprises fewer or no crosslinkable moieties, reducing the concentration of the epoxide resin and/or additional binder, using a bake cured an epoxide resin at temperatures less than is needed for curing (e.g., ambient conditions), not irradiating the coating, or a combination thereof.

(1) Ambient Condition Curing Epoxies

In certain embodiments, a curing agent suitable for curing at ambient conditions comprises an amine moiety such as a polyamine adduct, which is an epoxy resin modified to comprise an amine moiety, a polyamide, a ketimine, an aliphatic amine, or a combination thereof. Examples of an aliphatic amine include ethylene diamine (“EDA”), diethylene triamine (“DETA”), triethylene tetraamine (“TETA”), or a combination thereof. Selection of a polyamine adduct generally produces a film with excellent solvent resistance, corrosion resistance, acid resistance, flexibility, impact resistance, or a combination thereof. Selection of a polyamide generally produces a film with superior adhesion, particularly to a moist or poorly prepared surface, good solvent resistance, excellent corrosion resistance, good acid resistance, superior flexibility retention, superior impact resistance retention, or a combination thereof. A ketimine is a reaction product of a primary amine and a ketone, and produces a coating and/or film with similar properties as a polyamine or amine adduct. However, the pot life is longer with a ketimine, and moisture (e.g., atmospheric humidity) activates this cure agent. Examples of an epoxide selected for curing at ambient conditions includes a low mass epoxide resins with an n value from 0 to 2.0, including all intermediate ranges and combinations thereof. In certain embodiments, an epoxy resin may be selected with an epoxy equivalent weight of 182 to 1750, including all intermediate ranges and combinations thereof. In specific aspects, the greater the n value of an epoxide resin, the longer the pot life in a two-pack coating, the greater the coating leveling property, the lower the film solvent resistance, the lower the film chemical resistance, the greater the film flexibility, or a combination thereof. In certain aspects, an ambient curing epoxide coating is a two-pack coating, wherein the epoxide resin is in one container and the curing agent in a second container. In typical aspects, the pot life upon admixing the coating components is two hours to two days. An ambient cure epoxide may be selected for an industrial coating (e.g., industrial maintenance coating), a marine coating, an aircraft primer, a pipeline coating, a HIPAC, or a combination thereof.

(2) Bake Curing Epoxies

In other embodiments, a curing agent suitable for curing by baking includes an amino resin (e.g., a urea or melamine-based amino resin), a phenolic resin, or a combination thereof. Since baking is generally needed to promote film formation, an epoxy coating comprising such a curing agent typically is a one-pack coating. In certain embodiments, an epoxy resin may be selected with an epoxy equivalent weight of 1750 to 3050, including all intermediate ranges and combinations thereof. An epoxy resin coating that comprises an amino resin cure agent typically is selected for a lower cure temperature. Such a coating may be selected as a can coating, a metal coating, an industrial coating (e.g., equipment, appliances), or a combination thereof. An epoxy coating comprises an phenolic resin cure agent typically possesses greater chemical resistance and/or solvent resistance, and is typically selected for a can coating, a pipeline coating, a wire coating, an industrial primer, or a combination thereof. Examples of an epoxide selected for curing by baking includes a higher mass epoxide resins with an n value from 9.0 to 12.0, including all intermediate ranges and combinations thereof. In certain embodiments, a heat-cured epoxy coating is a water-borne coating. Such a water-borne coating comprises a higher mass epoxide resin modified to comprise a terpolymer that comprises monomers of styrene, methacrylic, acrylate, or a combination thereof, and an amino resin, a phenolic resin, or a combination thereof. Such a water-borne coating is typically selected as a can coating.

(3) Electrodeposition Epoxies

Another example of a water-borne epoxide coating is an electrodeposition epoxy coating. In certain embodiments, an epoxy resin may be selected with an epoxy equivalent weight of 500 to 1500, including all intermediate ranges and combinations thereof. An anionic and/or cationic epoxy resin is electrically attracted to a surface for application. The surface removed from the coating bath, and the coating is baked cured into a film upon the surface. Such a water-borne coating may be selected for an automotive primer, described elsewhere herein.

(4) Powder Coating Epoxies

An epoxy coating may be a powder coating, wherein the various nonvolatile coating components are admixed. Examples of typical admixed components include an epoxy resin, a curing agent, and a pigment, an additive, or a combination thereof. In certain embodiments, an epoxy resin may be selected with an epoxy equivalent weight of 550 to 750, including all intermediate ranges and combinations thereof. The mixture is then melted, cooled, and powderized. The powder coating is typically applied by attraction to an electrostatic charge of a surface. The thermosetting coating is cured by baking. An epoxy powder coating may be selected as a pipe coating, an electrical devise coating, an industrial coating (e.g., appliance coating, automotive coating, furniture coating), or a combination thereof.

(5) Cycloaliphatic Epoxies

A cycloaliphatic epoxy binder possesses a ring structure, rather than the linear structure for the epoxy embodiments described above. Examples of a cycloaliphatic epoxide is ERL-4221 (“3,4-epoxycyclohexylmethyl 3,4-epoxycyclohexane carboxylate”), which has an epoxy equivalent weight of 131 to 143, bis(3,4-epoxycyclohexylmethyl)adipate, which has an epoxy equivalent weight of 190 to 210, 2-(3,4-epoxycyclohexyl-5,5-spiro-3,4-epoxy)cyclohexane-m-dioxane, which has an epoxy equivalent weight of 133-154, 1-vinyl-epoxy-3,4-epoxycyclohexane, which has an epoxy equivalent weight of 70 to 74, or a combination thereof. Usually, a cycloaliphatic epoxy coating is combined with another binder, such as a polyol, a polyol modified to comprise a carboxyl moiety, or a combination thereof. An acid may be used to initiate crosslinking, particularly with a polyol. A cycloaliphatic epoxy polyol coating may comprise a triflic acid salt (e.g., diethylammonium triflate) to produce a one-pack coating with a pot life of up to eight months. In certain embodiments, a cycloaliphatic epoxy coating is a UV radiation cured coating, wherein the coating comprises a compound that converts to a strong acid upon UV irradiation (e.g., an onium salt). In certain aspects, a UV radiation cured cycloaliphatic epoxy coating is a one-pack coating. A UV radiation cured cycloaliphatic epoxy coating generally possesses excellent flame resistance, water resistance, or a combination thereof, and may be selected as a can coating or an electrical equipment coating. A compound comprising a carboxyl moiety (e.g., a carboxyl modified polyol) readily crosslinks with a cycloaliphatic epoxy binder. However, such a cycloaliphatic epoxy coating comprising such an additional binder generally has a short pot life (e.g., less than eight hours). In certain aspects, a cycloaliphatic epoxy carboxylic acid binder coating is a two-pack coating. A cycloaliphatic epoxy carboxylic acid polyol coating generally possesses excellent adhesion, toughness, gloss, hardness, solvent resistance, or a combination thereof.

i. Polyhydroxyether Binders

A polyhydroxyether binder (“polyhydroxyether resin,” “phenoxy binder,” “phenoxy”) chemically resembles a bisphenol A epoxy resin, though a polyhydroxyether binder lacks an epoxide moiety, and about 30 kDa in size. A polyhydroxyether coating is typically a thermoplastic coating. The polyhydroxyether binder comprises a hydroxyl moiety, and can be cross-linked with an additional binder such as an epoxide, a polyurethane comprising an isocyanate moiety, an amino resin, or a combination thereof. A thermosetting polyhydroxyether coating typically possesses excellent physical resistance properties, excellent chemical resistance, modest solvent resistance, or a combination thereof. In alternative embodiments, a polyhydroxyether binder temporary coating (e.g., a non-film forming coating) may be produced, for example, by selection of a polyhydroxyether binder that comprises fewer or no crosslinkable moieties, selection of an additional binder that comprises fewer or no crosslinkable moieties, reducing the concentration of the polyhydroxyether binder and/or additional binder, or a combination thereof.

j. Acrylic Resins

An acrylic resin (“acrylic polymer,” “acrylic binder,” “acrylic”) is a binder comprising a polymer of an acrylate ester monomer, a methacrylate ester monomer, or combination thereof. An acrylic-coating generally possesses a superior property of water resistance and/or exterior use durability than a polyester-coating. Other properties that an acrylic-coating typically possesses include color stability, chemical resistance, resistance to a UV light, or a combination thereof. An acrylic resin may further comprise an additional monomer to confer a desirable property to the resin, coating and/or film. For example, a styrene, a vinyltoluene, or a combination thereof, generally improve alkali resistance. Examples of such properties include the acrylic resin's chemical reactivity (e.g., cross-linkability), acidity, alkalinity, hydrophobicity, hydrophilicity, glass transition temperature, or a combination thereof. However, a thermoplastic acrylic film generally possesses poor solvent (e.g., acetone, toluene) resistance. Like other thermoplastic films, a thermoplastic acrylic film is generally easy to repair by application of additional acrylic coating to an area of solvent damage. An acrylic-coating is often suitable for various surfaces (e.g., metal), and examples of such coatings include an aerosol lacquer, an automotive coating, an architectural coating, a clear coating, a coating for external environment, an industrial coating, or a combination thereof. An acrylic resin may be used to prepare a thermoplastic coating, a thermosetting coating, or a combination thereof. In certain aspects, an acrylic-coating is selected for use as a thermosetting coating, particularly in embodiments for use upon a metal surface. Acrylic resins generally are soluble in a solvent with a similar solubility parameter. Examples of solvents typically used to dissolve an acrylic resin include an aromatic hydrocarbon (e.g., toluene, a xylene); a ketone (e.g., methyl ethyl ketone), an ester, or a combination thereof.

The thermoplastic and/or thermosetting properties of an acrylic resin are related to the monomers that are comprised in the selected resin. Examples of an acrylate ester monomer include a butylacrylate, an ethylacrylate (“EA”), ethylhexylacrylate (“EHA”), or a combination thereof. Examples of a methacrylate ester monomer include a butylmethacrylate (“BMA”), an ethylmethacrylate, a methylmethacrylate (“MMA”), or a combination thereof. Standards for physical properties, chemical properties, and/or procedures for empirically determining the purity/properties of various acrylic monomers (e.g., acrylate esters, 2-ethylhexyl acrylate, n-butyl acrylate, ethyl acrylate, methacrylic acid, acrylic acid, methyl acrylate) include, for example, “ASTM Book of Standards, Volume 06.04, Paint—Solvents; Aromatic Hydrocarbons,” D3362-93, D3125-97, D4415-91, D3541-91, D3547-91, D3548-99, D3845-96, D4416-89, and D4709-02, 2002).

In alternative embodiments, an acrylic resin temporary coating (e.g., a non-film forming coating) may be produced, for example, by selection of an acrylic resin that comprises fewer or no crosslinkable moieties, selection of an additional binder that comprises fewer or no crosslinkable moieties, reducing the concentration of the acrylic resin and/or additional binder, using a bake cured acrylic resin coating at temperatures less than is needed for curing (e.g., ambient conditions), selection of a size range for a thermoplastic acrylic resin coating that is less suitable for film formation (e.g., 1 kDa to 75 kDa), selection of a thermoplastic acrylic resin with T_{g} that is lower than the temperature ranges herein and/or 20° C. lower than the temperature range of use, or a combination thereof.

(1) Thermoplastic Acrylic Resins

A strait acrylic resin (“strait acrylic polymer,” “strait acrylic binder”) is a homopolymer or copolymer comprising an acrylate ester monomer and/or a methacrylate ester monomer. A strait acrylic resin may be used to formulate a thermoplastic coating, as cross-linking reactions are absent or limited without additional reactive moieties in the monomers. Generally, a thermoplastic film produced from an acrylic resin-coating will possess a lower elongation, an increased hardness, an increased tensile strength, greater UV resistance (e.g., chalk resistance), color retention, a greater T_{g}, or a combination thereof, with increasing methacrylate ester monomer content in the acrylic resin. However, the ester of a monomer may comprise various alcohol moieties, and an alcohol moiety of larger size generally reduces the T_{g}. Examples a T_{g} value for a homopolymer strait acrylic resins with the include −100° C., poly(octadecyl methacrylate); −72° C., poly(tetradecyl methacrylate); −65° C., poly(lauryl methacrylate); −60° C., poly(heptyl acrylate); −60° C., poly(n-decyl methacrylate); −55° C., poly(n-butyl acrylate); −50° C., poly(2-ethoxyethyl acrylate); −50° C., poly(2-ethylbutyl acrylate); −50° C., poly(2-ethylhexyl acrylate); −45° C., poly(propyl acrylate); −43° C., poly(isobutyl acrylate); −38° C., poly(2-heptyl acrylate); −24° C., poly(ethyl acrylate); −20° C., poly(n-octyl methacrylate); −20° C., poly(sec-butyl acrylate); −20° C., poly(ethylthioethyl methacrylate); −10° C., poly(2-ethylhexyl methacrylate); −5° C., poly(n-hexyl methacrylate); −3° C., poly(isopropyl acrylate); 6° C., poly(methyl acrylate); 11° C., poly(2-ethylbutyl methacrylate); 16° C., poly(cyclohexyl acrylate); 20° C., poly(n-butyl methacrylate); 35° C., poly(hexadecyl acrylate); 35° C., poly(n-propyl methacrylate); 43° C., poly(t-butyl acrylate); 53° C., poly(isobutyl methacrylate); 54° C., poly(benzyl methacrylate); 60° C., poly(sec-butyl methacrylate); 65° C., poly(ethyl methacrylate); 79° C., poly(3,3,5-trimethylcyclohexylmethacrylate); 81° C., poly(isopropyl methacrylate); 94° C., poly(isobornyl acrylate); 104° C., poly(cyclohexyl methacrylate); 105° C., poly(methyl methacrylate); 107° C., poly(t-butyl methacrylate); and 110° C., poly(phenyl methacrylate). Additionally, an estimated T_{g} of a copolymer comprising one or more monomers of an acrylate and/or methyacrylate monomer can be made by using the following equation: 1/T_{g}=W₁/T_{g1}+W₂/T_{g2}, wherein W₁ and W₂ are the are the molecular weight ratios of the first and second monomer, respectively; and wherein T_{g1} and T_{g2} are glass transition temperatures of the first and second monomer, respectively (Fox, T. G., 1956). For many embodiments (e.g., solvent-borne coatings), it is contemplated that a T_{g} of 40° C. to 60° C., including all intermediate ranges and combinations thereof, will be suitable.

The thermoplastic properties of an acrylic resin are also related to the molecular mass of the selected resin. Increasing the polymer size of an acrylic resin promotes physical polymer entanglement during film formation. Typically, a thermoplastic film produced from an acrylic-coating will possess a lower flexibility, an increased exterior durability, an increased hardness, an increased solvent resistance, an increased tensile strength, a greater T_{g}, or a combination thereof, with increasing polymer size of the acrylic resin. However, increasing polymer size of an acrylic resin generally increases viscosity of a solution comprising a dissolved acrylic resin, which may make application to a surface more difficult, such as cobwebbing of coating during spray application and the changes of film properties generally will reach a plateau at 100 kDa. In many embodiments, it is contemplated that an acrylic resin will range in mass from 75 kDa to 100 kDa, including all intermediate ranges and combinations thereof.

Examples of such a thermoplastic acrylic-coating include a lacquer. In specific facets, the lacquer possesses a good, high, or spectacular gloss. In specific aspects, such a thermoplastic acrylic-coating further comprises a pigment. In specific aspects, a wetting agent is less likely to be used in a coating comprising an acrylic resin and a pigment, due to the ease of dispersion of a pigment with an acrylic resin. In certain aspects, a thermoplastic acrylic-coating may be selected to coat a metal surface, a plastic surface, or a combination thereof. However, in particular aspects, a thermoplastic acrylic coating is an automotive coating. Such an automotive coating may comprise an acrylic binder with a high temperature T_{g} to produce a film of sufficient durability (e.g., hardness) for external use and contact with heated surfaces. In certain aspects, a thermoplastic acrylic coating comprises a binder with a T_{g} to 90° C. to 110° C., including all intermediate ranges and combinations thereof. In additional aspects, an automotive coating comprises a plasticizer, a metallic pigment, or a combination thereof. In specific aspects, a binder for an automotive coating comprises a methylmethacrylate ester monomer. In specific facets, an automotive coating comprises poly(methyl methacrylate).

(2) Water-Borne Thermoplastic Acrylic Coatings

The thermoplastic acrylic coatings described above are solvent-borne coatings. In other embodiments, a thermoplastic acrylic resin may be a waterborne coating. A water-borne acrylic (“acrylic latex”) typically is an emulsion, wherein the acrylic binder is dispersed in the liquid component. In general embodiments, an emulsifier (e.g., a surfactant) promotes dispersion. In certain embodiments, an acrylic latex coating comprises 0% to 20% coalescent per weight of binder. In many embodiments, it is contemplated that a water-borne acrylic resin will range in mass from 100 kDa to 1000 kDa, including all intermediate ranges and combinations thereof. In certain embodiments, a water-borne acrylic coating comprises an associative thickener (“rheology modifier”), which may enhance flow, brushability, splatter resistance, film build, or a combination thereof. A water-borne acrylic may be selected as an architectural coating. An associative thickener forms a network with acrylic resin latex particles by hydrophobic interactions. Hydroxyethyl cellulose (“HEC”) changes the coating rheology by promoting flocculation, which tends to reduce gloss, flow, or a combination thereof. Selection of an acrylic resin with smaller size, greater hydrophobicity, or a combination thereof, and an associative thickener may produce higher gloss, better flow, lower roller splatter, or a combination thereof.

(i) Architectural Coatings

A flat interior coating typically comprises a vinyl acetate and a lesser amount of acrylate (e.g., butyl acrylate) monomers, which generally produces a film with suitable scrub resistance. A copolymer of acrylate and methacrylate may be selected for a semigloss or gloss coating. In certain embodiments, the acrylate resin has a T_{g} to 20° C. to 50° C., including all intermediate ranges and combinations thereof. In some aspects, such a coating generally possesses good block resistance good print resistance, or a combination thereof. An acrylic resin that comprises a monomer that comprises a ureide moiety may be selected for enhanced film adhesion (e.g., to a coated surface), blistering resistance, or a combination thereof. An acrylic resin that comprises a styrene monomer may be selected for enhanced film water resistance.

An exterior latex coating typically produces a film with greater flexibility than an interior latex due to temperature changes and/or dimensional movement of a substrate (e.g., wood). In certain embodiments, the acrylic resin has a T_{g} to 10° C. to 35° C., including all intermediate ranges and combinations thereof. The selection of a T_{g} may be influenced by the selection of the amount particulate material (e.g., pigment) in the coating to achieve a particular visual appearance. For example, a higher the pigment volume content (“PVC”) that is typically selected to reduce gloss. However, to retain properties such as flexibility, a binder with a lower T_{g} may be selected for combination with the higher PVC. For example, a flat exterior latex coating generally possesses a pigment volume content of 40% to 60% and a T_{g} of 10° C. to 15° C., including all intermediate ranges and combinations thereof, respectively. In another example, a semigloss or gloss exterior latex binder of a coating generally possesses a T_{g} of 20° C. to 35° C., including all intermediate ranges and combinations thereof, respectively. In other embodiments, the exterior latex binder particle size is selected to be relatively small such as 90 nm to 110 nm, including all intermediate ranges and combinations thereof. In certain facets, a smaller latex particle size promotes adhesion of the coating and/or film, particularly to a surface that comprises a degraded (e.g., chalking) film. In certain other embodiments, a larger latex particle size may be selected to increase the coating and/or film's build (e.g., thickness). In certain aspects, a larger latex particle size ranges from, for example 325 nm to 375 nm, including all intermediate ranges and combinations thereof.

(ii) Industrial Coatings

A water-borne thermoplastic acrylic latex industrial coating typically comprises a binder with a T_{g} of 30° C. to 70° C., including all intermediate ranges and combinations thereof. Such a coating typically is applied to a metal surface, and thus often further comprises a surfactant, an additive, or a combination thereof to improve an anti-corrosion property. In specific aspects, the industrial coating comprises an anti-corrosion pigments, anti-corrosion pigment enhancers, or a combination thereof. In contrast, a water-borne acrylic latex industrial maintenance coating typically is similar to an exterior flat architectural coating in selection of binders, though they may comprise anti-corrosion pigments, anti-corrosion pigment enhancers, and other anti-corrosion components for use on a metal surface.

(3) Thermosetting Acrylic Resins

Unless otherwise noted, the following thermosetting acrylic resins and/or coatings are typically solvent-borne coatings. In certain embodiments an acrylic coating comprises a thermosetting acrylic resin. A thermosetting acrylic coating typically possesses superior hardness, superior toughness, superior temperature resistance, superior resistance to a solvent, superior resistance to a stain, superior resistance to a detergent, higher application of solids, relative to a thermoplastic acrylic coating. The average size of a thermosetting acrylic resin is typically less than a thermoplastic acrylic resin, which promotes a relatively lower viscosity and/or higher application of solids in a solution comprising a thermosetting acrylic resin. In certain embodiments, a thermosetting acrylic resin is from 10 kDa to 50 kDa, including all intermediate ranges and combinations thereof.

A thermosetting acrylic resin comprises a moiety capable of undergoing a cross-linking reaction. A monomer may comprise the moiety, and be incorporated into the polymer structure of an acrylic resin during resin synthesis (e.g., a styrene, a vinyltoluene), and/or the acrylic resin may be chemically modified after polymerization to comprise a chemical moiety. In additional embodiments, an acrylic resin may be selected to comprise chemical moieties, such as an amine, a carboxyl, an epoxy, a hydroxyl, an isocyanate, or a combination thereof, to confer a desirable property to the acrylic resin produced. Examples of such properties include the acrylic resin's chemical reactivity (e.g., crosslinkability), acidity, alkalinity, hydrophobicity, hydrophilicity, glass transition temperature, or a combination thereof. In general embodiments, an acrylic resin comprising a carboxyl moiety, a hydroxyl moiety, or a combination thereof, promotes a crosslinking reaction with another binder. In other embodiments, an acrylic resin may be chemically modified to comprise a methylol and/or methylol ether group, which is a resin capable of self-crosslinking.

(i) Acrylic-Epoxy Combinations

In certain embodiments, a thermosetting acrylic resin may be combined with an epoxide resin. In general embodiments, an acrylic resin comprising a carboxyl moiety may be selected for cross-linking with an epoxy resin. In specific aspects, an acrylic resin comprises 5% to 20% including all intermediate ranges and combinations thereof, of a monomer that comprises a carboxyl moiety, such as of an acrylic acid monomer, a methacrylic acid monomer, or a combination thereof. The carboxyl moiety may undergo a cross-linking reaction with an epoxide resin (e.g., a bisphenol A/epichlorohydrin epoxide resin) during film formation. In certain aspects, an epoxide resin cross-linked with an acrylic resin generally produces a film with good hardness, good alkali resistance, greater solvent resistance to a film, poorer UV resistance, or a combination thereof.

A thermosetting acrylic-epoxy coating may be selected for application to a metal surface. Examples of surfaces that an acrylic-epoxy coating is selected for use include an indoor surface, an indoor metal surface (e.g., an appliance), or a combination thereof. In certain aspects, an epoxide resin cross-linked with an acrylic resin generally produces a film with good hardness, good alkali resistance, greater solvent resistance to a film, poorer UV resistance, or a combination thereof. In some facets, an acrylic resin may be combined with an aliphatic epoxide resin to produce a film with relatively superior UV resistance than a bisphenol A/epichlorohydrin based epoxide resin. In another facet, an acrylic resin polymerized with an allyl glycidyl ether monomer, a glycidyl acrylate monomer, a glycidyl methacrylate monomer, or a combination thereof, may undergo a cross-linking reaction with an epoxide resin during film formation. In specific facets, a film produced from cross-linking an epoxide other than a bisphenol A/epichlorohydrin epoxide resin and an acrylic resin comprising an allyl glycidyl ether monomer, a glycidyl acrylate monomer, a glycidyl methacrylate monomer, or a combination thereof possesses a relatively superior UV resistance.

In certain embodiments, an acrylic epoxy coating comprises a catalyst to promote cross-linking during film formation. In specific aspects, the catalyst is a base such as a dodecyl trimethyl ammonium chloride, a tri(dimethylaminomethyl)phenol, a melamine-formaldehyde resin, or a combination thereof. In other embodiments, an acrylic epoxy coating is cured by baking at 150° C. to 190° C., including all intermediate ranges and combinations thereof. In particular aspects, film formation time of an acrylic epoxy coating is from 15 minutes to 30 minutes, including all intermediate ranges and combinations thereof. In certain embodiments, a thermosetting coating comprises an acrylic epoxide melamine-formaldehyde coating, wherein an acrylic resin, an epoxide resin and a melamine-formaldehyde resin undergo cross-linking during film formation.

(ii) Acrylic-Amino Combinations

In other embodiments, a thermosetting acrylic resin may be combined with an amino resin. In general embodiments, an acrylic resin comprising an acid (e.g., carboxyl) moiety, a hydroxyl moiety, or a combination thereof, may be selected for cross-linking with an amino resin. An acrylic amino coating, wherein the acrylic resin comprises an acid moiety, may be cured by baking at, for example 150° C. for 30 minutes. However, an acid moiety acrylic amino coating typically undergoes a greater degree of reactions between amino resins, which reduces properties such as toughness. In specific aspects, an acrylic resin comprises a monomer that comprises a hydroxyl moiety such as a hydroxyethyl acrylate (“HEA”), a hydroxyethyl methacrylate (“HEMA”), or a combination thereof. An acrylic amino coating, wherein the acrylic resin comprises a hydroxyl moiety, typically comprises an acid catalyst to promote curing by baking at, for example 125° C. for 30 minutes. An acrylic amino coating, wherein the amino resin was prepared from urea, generally produces a film with lower gloss, less chemical resistance, or a combination thereof, than an amino resin prepared from another nitrogen compound. Selection of a melamine and/or benzoguanamine based amino coating generally produces a film with excellent weathering resistance, excellent solvent resistance, good hardness, good mar resistance, or a combination thereof, and such an acrylic amino coating may be selected for an automotive topcoat.

(iii) Acrylic-Urethane Combinations

In other embodiments, a thermosetting acrylic resin may be combined with a urethane resin. In general embodiments, an acrylic resin comprising an acid moiety, a hydroxyl moiety, or a combination thereof, may be selected for crosslinking with a urethane resin. In specific embodiments, an acrylic resin comprises a hydroxyl moiety, such as, for example, a moiety provided by a HEA monomer, a HEMA monomer, or a combination thereof. Selection of an aliphatic isocyanate urethane (e.g., hexamethylene diisocyanate based) generally produces a film with superior color, weathering, or a combination thereof relative to other urethanes. An acrylic urethane coating may comprise a catalyst, such as, for example, triethylene diamine, zinc naphthenate, dibutyl tin-di-laurate, or a combination thereof. An acrylic urethane coating cures at ambient conditions. However, an acrylic urethane coating typically is a two-pack coating to separate the reactive binders until application. An acrylic urethane coating generally produces a film with good weathering, good hardness, good toughness, good chemical resistance, or a combination thereof. An acrylic urethane coating may be selected an aircraft coating, an automotive coating, an industrial coating (e.g., an industrial maintenance coating), or a combination thereof.

(iv) Water-Borne Thermosetting Acrylics

In other embodiments, a thermosetting acrylic coating may be a waterborne coating (e.g., a latex coating). Typically, such a thermosetting acrylic coating comprises an acrylic resin with a hydroxyl moiety, an acid moiety, or a combination thereof. An acrylic resin may further comprise an additional monomer such as a styrene, a vinyltoluene, or a combination thereof. The acrylic resin typically is combined in a coating with an amino resin, an epoxy resin, or a combination thereof as previously described. A film produced from a water-borne thermosetting acrylic coating is similar in properties as a solvent-borne counterpart. Such a coating may be selected for surfaces such as masonry, wood, metal, or a combination thereof.

k. Polyvinyl binders

A polyvinyl binder (“polyvinyl,” “vinyl binder,” “vinyl”) is a binder comprising a polymer of a vinyl chloride monomer, a vinyl acetate monomer, or combination thereof. A solvent-borne polyvinyl coating may comprise a ketone, ester, chlorinated hydrocarbon, nitroparaffin, or a combination thereof, as a solvent. A solvent-borne polyvinyl coating may comprise a hydrocarbon (e.g., aromatic, aliphatic) as a diluent. A polyvinyl binder is generally insoluble in an alcohol, however, in embodiments wherein a solvent-borne polyvinyl coating that comprises an additional alcohol soluble binder, alcohol may comprise 0% to 20% of the liquid component. In embodiments wherein solvent-borne polyvinyl coating is cured by baking, a glycol ether and/or glycol ester may be used in the liquid component to enhance a rheological property. In other embodiments, the liquid component of a polyvinyl coating may comprise a plasticizer (e.g., a phthalate, a phosphate, a glycol ester), wherein the plasticizer if 1 to 25 parts per hundred parts polyvinyl binder, including all intermediate ranges and combinations thereof, for a non-plastisol or non-organosol coating. A polyvinyl-coating may be used to prepare a thermoplastic coating, a thermosetting coating, or a combination thereof. In specific aspects, a thermoplastic polyvinyl binder coating possesses a T_{g} of 50° C. to 85° C., including all intermediate ranges and combinations thereof. However, in some aspects, a polyvinyl-coating/film possesses moderate resistance to heat, UV irradiation, or a combination thereof. In specific aspects, a polyvinyl-coating comprises a light stabilizer, a pigment, or a combination thereof. In particular facets, the light stabilizer, the pigment (e.g., titanium dioxide), or the combination thereof, improves the polyvinyl-coating and/or film's resistance to heat, UV irradiation, or a combination thereof.

In embodiments wherein a polyvinyl coating comprises a solvent-borne coating, it is contemplated that a polyvinyl resin will range in mass from 2 kDa to 45 kDa, including all intermediate ranges and combinations thereof. A typical solvent-borne polyvinyl coating comprises a polyvinyl resin, a liquid component wherein the liquid component comprises a solvent, and a plasticizer. A solvent-borne polyvinyl coating may additionally comprise a colorizing agent (e.g., a pigment), a light stabilizer, an additional binder, a cross-linker, or a combination thereof.

A polyvinyl binder typically possesses excellent adhesion for a plastic surface, an acrylic and/or acrylic coated surface, paper, or a combination thereof. A thermoplastic polyvinyl coating may be selected as a lacquer, a topcoat of a can coating (e.g., can interior surface), or a combination thereof. In some embodiments, an polyvinyl-coating may be selected to produce a film with such properties, for example, as excellent water resistance, excellent resistance to various solvents (e.g., an aliphatic hydrocarbon, an alcohol, an oil), excellent resistance to acid pH, excellent resistance to basic pH, inertness relative to food, or a combination thereof.

In many aspects, a polyvinyl resin is a copolymer that comprises a combination of a vinyl chloride monomer and vinyl acetate monomer. Often during resin synthesis (e.g., polymerization), a polyvinyl resin is prepared to further comprise monomers with specific chemical moieties to confer a property such as solubility in water, solubility in a solvent, compatibility with another coating component (e.g., a binder), or a combination thereof. In certain embodiments, a polyvinyl resin comprises a monomer comprising carboxyl moiety, a hydroxyl moiety (e.g., a hydroxyalkyl acrylate monomer), a monomer comprising an epoxy moiety, a monomer comprising a maleic acid, or a combination thereof. A carboxyl moiety may confer an increased adhesion property (e.g., excellent adhesion to metal). However, a polyvinyl resin comprising a carboxyl moiety is generally not compatible with a basic pigment. A thermosetting polyvinyl coating comprising a polyvinyl binder that comprises a carboxyl moiety and a polyvinyl binder that comprises an epoxy moiety generally possesses one or more excellent physical properties (e.g., flexibility), and may be selected as a coil coating. A hydroxyl moiety may confer cross-linkability, compatibility with another coating component, an increased adhesion property (e.g., good adhesion to aluminum), or a combination thereof. Additionally, after polymer synthesis, a polyvinyl resin can be chemically modified to comprise such a specific chemical moiety. In some embodiments, a polyvinyl resin is chemically modified to comprise a secondary hydroxyl moiety, an epoxy moiety, a carboxyl moiety, or a combination thereof. A polyvinyl resin comprising a secondary hydroxyl moiety may be combined with another binder such as an alkyd, a urethane, an amino-formaldehyde, or a combination thereof. A thermosetting polyvinyl amino-formaldehyde coating comprising a polyvinyl binder that comprises a hydroxyl moiety generally possesses good corrosion resistance, water resistance, solvent resistance, chemical resistance, and may be selected as a can coating, a coating for an interior wood surface, or a combination thereof. Standards for physical properties, chemical properties, and/or procedures for testing the purity/properties of various polyvinyl monomers (e.g., vinyl acetate) and polyvinyl resins (e.g., polymer components, polymer mass, shear viscosity for a higher mass resin, chlorine content) are described, for example, in “ASTM Book of Standards, Volume 06.04, Paint—Solvents; Aromatic Hydrocarbons,” D2190-97, D2086-02, D2191-97, and D2193-97, 2002; “ASTM Book of Standards, Volume 06.03, Paint—Pigments, Drying Oils, Polymers, Resins, Naval Stores, Cellulosic Esters, and Ink Vehicles,” D4368-89, D3680-89, and D1396-92, 2002; and in “ASTM Book of Standards, Volume 06.01, Paint—Tests for Chemical, Physical, and Optical Properties; Appearance,” D2621-87, 2002.

In alternative embodiments, a polyvinyl resin temporary coating (e.g., a non-film forming coating) may be produced, for example, by selection of a polyvinyl resin that comprises fewer or no crosslinkable moieties, selection of an additional binder that comprises fewer or no crosslinkable moieties, reducing the concentration of the polyvinyl resin and/or additional binder, using a bake cured polyvinyl resin coating at temperatures less than is needed for curing (e.g., ambient conditions), selection of a size range for a plastisol or organisol polyvinyl resin coating that is less suitable for film formation (e.g., 1 kDa to 60 kDa), selection of a polyvinyl resin with T_{g} that is lower than the temperature ranges herein and/or 20° C. lower than the temperature range of use, or a combination thereof.

(1) Plastisols and Organisols

A polyvinyl resin of 60 kDa to 110 kDa, including all intermediate ranges and combinations thereof, may be selected for use as an organosol or a plastisol. A plastisol is a coating comprising a vinyl homopolymer binder and a liquid component, wherein the liquid component generally comprises a plasticizer comprising a minimum of 55 parts or more of plasticizer per hundred parts of homopolymer binder in the coating. In certain embodiments, a plastisol comprises, by weight, 0% to 10% including all intermediate ranges and combinations thereof, of a thinner (e.g., an aliphatic hydrocarbon). A plastisol coating typically comprises an additional vinyl binder. A plastisol may comprise a pigment, however, a low oil absorption pigment may be used to avoid undesirable increase in coating viscosity given the liquid component used for a plastisol.

An organosol is similar to a plastisol, except the less than 55 parts of plasticizer per hundred parts of homopolymer binder is used in the coating. In typical embodiments, the liquid component comprises a weak solvent that may act as a dispersant and a thinner (e.g., a hydrocarbon). In typical aspects, the reduced content of plasticizer produced a film with a superior hardness property relative to a plastisol. In additional embodiments, the nonvolatile component of an organisol is 50% to 55%, including all intermediate ranges and combinations thereof. An organosol coating typically comprises a second binder. In specific aspects, the second binder is a vinyl copolymer, an acrylic, or a combination thereof. In certain aspects, the second binder comprises a carboxyl moiety, a hydroxyl moiety, or a combination thereof. In further aspects, an organisol may comprise a third binder. In specific facets, the third binder comprises an amino resin, a phenolic resin prepared from formaldehyde, or a combination thereof. In additional facets, a second binder that comprises a hydroxyl moiety may undergo a thermosetting cross-linking reaction with a third binder. An organisol may comprise a pigment suitable for general polyvinyl coatings.

A plastisol or organisol typically is cured by baking. In general embodiments, baking is at a temperature of 175° C. to 180° C., including all intermediate ranges and combinations thereof. In general embodiments, a plastisol or organisol comprises a heat stabilizer. The heat stabilizer may protect a vinyl binder during baking. Examples of a suitable heat stabilizer include a combination of a metal salt of an organic acid and an epoxidized oil or a liquid epoxide binder. However, in an embodiment wherein the plastisol or organisol comprises a binder that comprises a carboxyl moiety, a metal salt is less likely to be used due to possible gellation of the coating, and may be substituted with a merapto tin and/or tin ester compound.

In embodiments wherein a plastisol or organisol comprise a binder with good adhesion properties for a surface such as a binder comprising carboxy moiety, the plastisol or organisol may be used as a single layer coating. For example, such an organisol may be selected to coat the end of a can. However, a plastisol or organisol typically is part of a multicoat system that comprises a primer to promote adhesion. In specific aspects, the primer comprises a vinyl resin comprising a carboxy moiety. In specific facets, the primer further comprises a thermosetting binder such as an amino-formaldehyde, phenolic, or a combination thereof, to enhance solvent resistance. In certain facets, it is contemplated that a primer or other coat layer of a multicoat system possesses good solvent resistance to the plasticizers of the organosol and/or plastisol coat layer.

(2) Powder Coatings

A polyvinyl binder may be selected as a powder coating. Typically, coating components such as a polyvinyl binder and a plasticizer, colorizing agent, additive, or a combination thereof, admixed to prepare a powder coating. Such a powder coating is usually applied by a fluidized bed applicator, a spray applicator, or a combination thereof. In some aspects, the coating components are melted then ground into a powder. Such a powder coating is usually applied by an electrostatic spray applicator. The coating is cured by baking. A polyvinyl powder coating may be selected to coat a metal surface.

(3) Water-Borne Coatings

The previous discussions of polyvinyl coatings focused upon solvent-borne and powder coatings. A polyvinyl binder with a T_{g} of 75° C. to 85° C., including all intermediate ranges and combinations thereof, may be selected for use in a dispersion waterborne coating. The liquid component may comprise a cosolvent such as a glycol ether, a plasticizer, or a combination thereof. Examples of a cosolvent include ethylene glycol monobutyl ether. The dispersion water-borne polyvinyl coating may be used as described for a solvent-borne polyvinyl coating. In another example, an organisol may be prepared with a plasticizer as a latex coating. Such a latex is suitable for selection as a primer coating. The latex coating is cured by baking.

1. Rubber Resins

In certain embodiments, a coating may comprise a rubber resin as a binder. A rubber may be either obtained from a biological source (“natural rubber”), synthesized from petroleum (“synthetic rubber”), or a combination thereof. Examples of synthetic rubber include polymers of styrene monomers, butadiene monomers, or a combination thereof. In alternative embodiments, a rubber temporary coating (e.g., a non-film forming coating) may be produced, for example, by selection of rubber resin that comprises fewer or no crosslinkable moieties, selection of an additional binder that comprises fewer or no crosslinkable moieties, reducing the concentration of the rubber resin and/or additional binder, or a combination thereof.

(1) Chlorinated Rubber Resins

In general embodiments, a rubber resin comprises a chlorinated rubber resin, wherein a rubber isolated from a biological source has been chemically modified by reaction with chlorine to produce a resin comprising 65% to 68% chlorine by weight, including all intermediate ranges and combinations thereof. A chlorinated rubber resins generally are in a molecular weight range of 3.5 kDa to 20 kDa, including all intermediate ranges and combinations thereof. A chlorinated rubber coating may comprise another binder, such as, for example, an acrylic resin, an alkyd resin, a bituminous resin, or a combination thereof. In specific aspects, a chlorinated rubber resin comprises 10% to 50%, by weight, including all intermediate ranges and combinations thereof, of the binder when in combination with an acrylic resin, an alkyd resin, or a combination thereof. In general embodiments, a chlorinated rubber coating is a solvent-borne coating. In certain aspects, a chlorinated rubber coating comprises a liquid component, such as, for example, a solvent, a diluent, a thinner, a plasticizer, or a combination thereof. A chlorinated rubber coating may be a thermoplastic coating. To reduce the T_{g} of a film produced from a chlorinated rubber resin, the liquid component generally comprises a plasticizer. In certain aspects, a chlorinated rubber coating comprises 30% to 40%, by weight, including all intermediate ranges and combinations thereof, of plasticizer. In certain facets, a plasticizer is selected for water resistance (e.g., hydrolysis resistance) such as a bisphenoxyethylformal. In certain facets, a chlorinated rubber coating comprises light stabilizer, an epoxy resin, an epoxy plasticizer (e.g., epoxidized soybean oil), or combination thereof, to chemically stabilize a chlorinated resin, coating and/or film. In other embodiments, a chlorinated rubber coating comprises a pigment, an extender, or a combination thereof. In particular aspects, the pigment is a corrosion resistant pigment. A chlorinated rubber film are generally has good chemical resistance (e.g., acid resistance, alkali resistance), water resistance, or a combination thereof. Coatings comprising chlorinated rubber resins may be used, for example, on surfaces that contact gaseous, liquid and/or solid external environments. Examples of such uses include a coating for an architectural coating (e.g., a masonry coating), a traffic marker coating, a marine coating (e.g., a marine vehicle, a swimming pool), a metal primer, a metal topcoat, or a combination thereof.

(2) Synthetic Rubber Resins

Examples of synthetic rubber include polymers comprising a styrene monomer, a methylstyrene (e.g., α-methylstyrene) monomer, or a combination thereof. A polystyrene and/or polymethylstyrene coating may be a solvent-borne coating. Examples of a solvent include an aliphatic hydrocarbon, an aromatic hydrocarbon, a ketone, an ester, or a combination thereof. A polystyrene and/or polymethylstyrene coating may possess good water resistance, good chemical resistance, or a combination thereof. A polystyrene and/or polymethylstyrene coating may be selected as a primer, a lacquer, a masonry coating, or a combination thereof. A polystyrene homopolymer has a T_{g} of 100° C., and in certain embodiments, a polystyrene coating is bake cured. Standards for physical properties, chemical properties, and/or procedures for testing the purity/properties of a styrene monomer, a methylstyrene monomer, (e.g., α-methylstyrene), a resin comprising a styrene and/or methylstyrene monomer, are described, for example, in “ASTM Book of Standards, Volume 06.04, Paint—Solvents; Aromatic Hydrocarbons,” D2827-00, D6367-99, D6144-97, D4590-00, D2119-96, D2121-00, and D2340-96, 2002.

Similar to the variability of T_{g} previously described for a thermoplastic acrylic resin, a styrene copolymer with a lower a T_{g} than polystyrene or other altered properties can be produced from polymerization with other monomers such as a butadiene monomer, an acrylic monomer, a maleate ester, an acrylonitrile, an allyl alcohol, a vinyltoluene, or a combination thereof. For example, a butadiene monomer decreases lightfastness, but confers self-crosslinkability to the resin. In another example, an acrylic resin increases the resin's solubility in an alcohol. In a further example, an allyl alcohol monomer confers crosslinkability in combination with a polyol. In certain embodiments, a styrene-butadiene copolymer resin may be selected. In certain aspects, a styrene-butadiene resin comprises a carboxyl moiety to improve an adhesion property, dispersibility in a liquid component, or a combination thereof. In particular facets, a styrene-butadiene coating comprises an emulsifier to increase dispersion in a liquid component, a light stabilizer, or a combination thereof. A styrene-butadiene coating may be a thermosetting coating, due to oxidative crosslinking of a butadiene double bond moiety. However, styrene-butadiene film may have poor chalking resistance, poor color stability, poor UV resistance, or a combination thereof. A styrene-butadiene coating may be selected as a corrosion resistant primer, a wood primer, or a combination thereof. A styrene-vinnyltoluene-acrylate copolymer coating may be selected for an exterior coating, a traffic marker paint, a metal coating (e.g., a metal lacquer), a masonry coating, or a combination thereof.

m. Bituminous Binders

A bituminous binder (“bituminous”) is a binder comprising a hydrocarbon soluble in carbon disulfide, is black or dark colored, and is obtained from a bitumen deposit and/or as a product of petroleum processing. A bituminous binder typically is used in asphalt, tar, and other construction materials. However, in certain embodiments, a bituminous binder may be used in a coating, particularly in embodiments wherein good resistance to a chemical such as a petroleum based solvent, an oil, water, or a combination thereof, is desired. Examples of a bituminous binder include a coal tar, a petroleum asphalt, a pitch, an asphaltite, or a combination thereof. In certain embodiments, a coal tar and/or pitch is combined with an epoxy resin to form a thermosetting coating. Such as coating may be selected as a pipeline coating. In other embodiments, an asphaltite and/or petroleum asphalt may be selected for use as an automotive coating (e.g., an underbody part coating). An asphaltite and/or petroleum asphalt coating may further comprise an additional binder such as an epoxy. In certain aspects, an asphaltite and/or petroleum asphalt coating is a solvent-borne coating. In specific aspects, an asphaltite and/or petroleum asphalt coating comprises a plasticizer. In further aspects, an asphaltite and/or petroleum asphalt coating comprises a wax to increase abrasion resistance.

In further embodiments, a bituminous coating may be selected as a roof coating. Typically, a bituminous roof coating comprises an extender, a thixotrope, or a combination thereof. Examples of a thixotrope additive include asbestos, a silicon extender, a celluosic, a glass fiber, or a combination thereof. In some aspects, a bituminous roof coating comprises a solvent-borne coating or a water-borne coating. Examples of solvents that may be selected include a mineral spirit, an aliphatic hydrocarbon (e.g., a naphtha, a mineral spirit), an aromatic solvent (e.g., xylene, toluene) or a combination thereof. A bituminous roof coating may be selected as a primer, a topcoat, or a combination thereof. A bituminous roof topcoat typically further comprises a metallic pigment.

In certain aspects, a solvent-borne or water-borne bituminous coating is an emulsion comprising water and a bituminous binder. In specific facets, the emulsion further comprises a solvent, an extender (e.g., a silica), an emusifier (e.g., a surfactant), or a combination thereof. The extender typically functions to stabilize the emulsion. In particular facets, the emulsion bituminous coating is a roof coating, a road coating, a sealer, a primer, a topcoat, or a combination thereof. In facets wherein an emulsion bituminous coating is selected as a sealer, an additional binder may be added to increase solvent resistance.

In alternative embodiments, a bituminous temporary coating (e.g., a non-film forming coating) may be produced, for example, by selection of an additional binder that comprises fewer or no crosslinkable moieties, reducing the concentration of the bituminous resin and/or additional binder, or a combination thereof.

n. Polysulfide Binders

A polysulfide binder is a polymer produced from a reaction of sodium polysufide, bis(2-chlorethyl)formal and 1,2,3-trichloropropane. Typically, a polysulfide binder is 1 kDa to 8 kDa, including all intermediate ranges and combinations thereof. A polysulfide binder comprises a thiol (“mercaptan”) moiety capable of crosslinking with an additional binder. A polysulfide may undergo crosslinking by an oxidative reaction with an additional binder comprising a peroxide (e.g., dicumen hydroperoxide), a manganese dioxide, p-quinonedioxime, or a combination thereof. A polysulfide binder may be crosslinked with a glycidyl epoxide, though a tertiary amine may be used as part of the coating to promote this reaction. A polysulfide may undergo crosslinking with a binder comprising an isocyanate moiety, though the binder may comprise a plurality of isocyanates. A polysulfide film typically possesses excellent UV resistance, good general weatherability properties, good chemical resistance, or a combination thereof.

In alternative embodiments, a polysulfide temporary coating (e.g., a non-film forming coating) may be produced, for example, by selection of an additional binder that comprises fewer or no crosslinkable moieties, reducing the concentration of the bituminous resin and/or additional binder, or a combination thereof.

o. Silicone Binders

The previous described binders are molecules based on carbon, and are considered herein as “organic binders”. A silicone binder (“silicone”) is a binder molecule based on silicone. Examples of a silicone binder include a polydimethyllsiloxane and a methyltriacetoxy silane, a methyltrimethoxysilane, a methyltricyclorhexylaminosilane, a fluorosilicone, a trifluoropropyl methyl polysiloxane, or a combination thereof. In general embodiments, a silicone binder comprises a crossreactive silicon moiety, examples of which are described below. A silicone coating may be selected for excellent resistance to irradiation (e.g., UV, infrared, gamma), excellent weatherability, excellent biodegradation resistance, flame resistance, excellent dielectric property, which is poor electrical conductivity with little detrimental effect on an electrostatic field, or a combination thereof. In specific aspects, a silicon coating is an industrial coating. In particular facets, a silicon coating is applied to an appliance part, a furnace part, a jet engine part, an incinerator part, or a missile part. In other embodiments, a silicon coating comprises an organic binder. In particular aspects, a silicon organic binder coating possesses superior heat resistance to an organic binder coating. In other aspects, the greater the silicon binder to organic binder ratio, the greater the crosslinking reactions, greater film hardness, reduced flexibility, or a combination thereof.

In general embodiments, a silicone coating is a thermosetting coating. Often, a silicon coating is a multi-pack coating due to a limited pot life when the coating components are admixed. The crosslinking reaction depends upon the binder's specific silicon moiety. A plurality of binders may be used, each comprising one or more crosslinking moieties. A binder comprising crosslinking SiOH and HOSi moieties generally comprises a cure agent such as a lead octoate, a zinc octoate, or a combination thereof. In general aspects, the thermosetting SiOH and HOSi silicon coating is bake cured (e.g., 250° C. for one hour). A binder comprising crosslinking SiOH and HSi moieties typically comprises a tin catalyst. A binder comprising crosslinking SiOH and ROSi moieties, wherein RO is an alkoxy moiety, also typically comprises a tin catalyst. A coating prepared using SiOH and ROSi silicon binder typically further comprises an iron oxide, a glass microballon, or a combination thereof to improve heat resistance. This type of silicon may be selected for rocket and jet engine parts. A binder comprising crosslinking SiOH and CH₃COOSi moieties is moisture cured, and typically comprises a tin catalyst (e.g., an organotin compound). A binder comprising crosslinking SiOH and R₂NOSi moieties, wherein R₂NO is an oxime moiety, is also moisture cured, and typically comprises a tin catalyst. The moisture cured silicon coatings may be selected for one-pack silicon coatings, though film formation is generally slower than other types of silicon thermosetting coatings. A binder comprising crosslinking SiCH═CH₂ and R₂NOSi moieties, wherein R₂NO is an oxime moiety, typically comprises a platinum catalyst, and may be bake cured. A film produced by a SiCH═CH₂ and R₂NOSi silicon coating possesses excellent toughness, flame resistance, or a combination thereof. Such a coating may be selected for a rocket part. However, coating components such as a rubber, a tin compound (e.g., an organotin), or a combination thereof, may inhibit platinum catalyzed film formation in this silicon coating.

In certain embodiments, a silicone coating is a solvent-borne coating. Examples of liquid components that may function as a silicon solvent include a chlorinated hydrocarbon (e.g., 1,1,1-trichloroethane), an aromatic hydrocarbon (e.g., a VMP naphtha, xylene), an aliphatic hydrocarbon, or a combination thereof. A silicone binder typically is insoluble or poorly soluble in an oxygenated compound such as an alcohol, a ketone, or a combination thereof, of relatively low molecular weight (e.g., ethanol, isopropanol, acetone). However, a fluorosilicone, which is a silicone binder that comprises a fluoride moiety, may be combined with a liquid component comprising a ketone such as methyl ethyl ketone, methyl isobutyl ketone, or a combination thereof. A fluorosilicone binder may be selected for producing a film with excellent solvent resistance. A silicon coating often comprises a pigment. In specific embodiments, a pigment comprises zinc oxide, titanium dioxide, zinc orthotitanate, or a combination thereof, which may improve a film's resistance to extreme temperature variations, such as those of outerspace. In specific embodiments, a silicon coating may comprise a silica extender (e.g., fumed silica), which often increases durability.

In certain embodiments, a silicon binder comprises a trifluoropropyl methyl polysiloxane binder. In certain aspects, a trifluoropropyl methyl polysiloxane binder may be selected for producing a film with excellent resistance to petroleum products (e.g., automotive fuel, aircraft fuel), but poor resistance to an acid or an alkali, particularly at baking conditions.

In alternative embodiments, a silicon temporary coating (e.g., a non-film forming coating) may be produced, for example, by selection of an additional binder that comprises fewer or no crosslinkable moieties, reducing the concentration of the silicon resin and/or additional binder, using a bake-cured silicon coating at non-baking conditions, inclusion of a rubber, a tin compound (e.g., an organotin), or a combination thereof.

2. Liquid Components

A liquid component is a chemical composition that is in a liquid state while comprised in a coating and/or film. A liquid component is typically added to a coating composition, for example, to improve a rheological property for ease of application, alter the period of time that thermoplastic film formation occurs, alter an optical property (e.g., color, gloss) of a film, alter a physical property of a coating (e.g., reduce flammability) and/or film (e.g., increase flexibility), or a combination thereof.

Often a liquid component comprises a volatile liquid that is partly or fully removed (e.g., evaporated) from the coating during film formation. In many embodiments, 0% to 100%, including all intermediate ranges and combinations thereof, of the liquid component is lost during film formation. Examples of a volatile liquid include a volatile organic compound (“VOC”), water, or a combination thereof. Various environmental laws and regulations have encouraged the reduction of volatile organic compound use in coatings [see “Paint and Coating Testing Manual, Fourteenth Edition of the Gardner-Sward Handbook,” (Koleske, J. V. Ed.), pp. 3-12, 1995]. As a consequence, a coating may comprise a solvent-borne coating, which typically comprises a VOC and was the coating usually selected prior to enactment of the environmental laws, a high solids coating, which is generally a solvent-borne coating formulated with a minimum amount of a VOC, a water-borne coating, which comprises water and typically even less VOC, or a powder coating, which comprises little or no VOC.

In many embodiments, a liquid component may comprise a liquid composition classified based upon function such as a solvent, a thinner, a diluent, a plasticizer, or a combination thereof. A solvent is a liquid component used to dissolve one or more coating components. A thinner is a liquid component used to reduce the viscosity of a coating, and often additionally confers one or more properties to the coating, such as, for example, dissolving a coating component (e.g., a binder), wetting a colorizing agent, acting as an antisettling agent, stabilizing a coating in storage, acting as an antifoaming agent, or a combination thereof. A diluent is a liquid component that does not dissolve a binder.

Liquid components can be classified, based on their chemical composition, as an organic compound, an inorganic compound, or a combination thereof. In many embodiments, organic compounds include a hydrocarbon, an oxygenated compound, a chlorinated hydrocarbon, a nitrated hydrocarbon, a miscellaneous organic liquid component, or a combination thereof. A hydrocarbon consists of or consists essentially of one or more carbon and/or hydrogen atoms. Examples of a hydrocarbon include an aliphatic hydrocarbon, an aromatic hydrocarbon, a naphthene, a terpene, or a combination thereof. An oxygenated compound comprises of one or more carbon, hydrogen and/or oxygen atoms. Examples of an oxygenated compound include an alcohol, an ether, an ester, a glycol ester, a ketone, or a combination thereof. A chlorinated hydrocarbon comprises one or more carbon, hydrogen and/or chlorine atoms, but does not comprise an oxygen atom. A nitrated hydrocarbon comprises one or more carbon, hydrogen and/or nitrogen atoms, but does not comprise an oxygen atom. A miscellaneous organic liquid component is a liquid other than a chlorinated hydrocarbon and/or a nitrated hydrocarbon that comprises one or more carbon, hydrogen and/or other atoms. In certain aspects, a miscellaneous organic liquid component does not comprise an oxygen atom. In typical embodiments, inorganic compounds include ammonia, hydrogen cyanide, hydrogen fluoride, hydrogen cyanide, sulfur dioxide, or a combination thereof. However, an inorganic compound generally is used at temperatures less than ambient conditions, and at pressures greater than atmospheric pressure.

In certain embodiments, a liquid component may comprise an azeotrope. An azeotrope (“azeotropic mixture”) is a solution of two or more liquid components at concentrations that produces a constant boiling point for the solution. An azeotrope BP (“A-BP”) is the boiling point of an azeotrope. Often, the boiling point (“BP”) of the majority component of an azeotrope is higher than the A-BP, and in some embodiments, such an azeotrope evaporates from a coating faster than a similar coating that does not comprise the azeotrope. However, in some aspects, a coating comprising an azeotrope with a superior evaporation property may possess a lower flash point temperature, a lower explosion limit, a reduced coating flow, greater surface defect formation, or a combination thereof, relative to a similar coating that does not comprise the azeotrope. Alternatively, an azeotrope may be selected for embodiments wherein a component's BP is increased. In specific aspects, a coating comprising such an azeotrope may have a relatively slower evaporation rate than a similar coating that does not comprise the azeotrope. It is contemplated that the greater the percentage of liquid component is an azeotrope, the greater the conference of an azeotrope's property to a coating. Thus, a specific range of 50% to 100%, 90% to 100%, or 95% to 100%, including all intermediate ranges and combinations thereof, may be sequentially selected in embodiments wherein an azeotrope's property is desired as a property of a coating.

In some embodiments, a chemically non-reactive (“inert”) liquid component may be selected. Typically, a liquid component is selected that is inert relative to a particular chemical reaction to prevent an undesirable chemical reaction with other coating components. An example of such an undesirable chemical reaction is a binder-liquid component reaction that is inhibitory to a desired binder-binder film-formation reaction. Examples of a liquid component that are generally inert in an acetal formation reaction include benzene, hexane, or a combination thereof. An example of a liquid component that is generally inert in a decarboxylation reaction includes quinoline. Examples of a liquid component that are generally inert in a dehydration reaction include benzene, toluene, xylene, or a combination thereof. An example of a liquid component that is generally inert in a dehydrohalogenation reaction includes quinoline. Examples of a liquid component that are generally inert in a diazonium compound coupling reaction include ethanol, glacial acetic acid, methanol, pyridine, or a combination thereof. Examples of a liquid component that are generally inert in a diazotization reaction include benzene, dimethylformamide, ethanol, glacial acetic acid, or a combination thereof. Examples of a liquid component that are generally inert in an esterification reaction include benzene, dibutyl ether, toluene, xylene, or a combination thereof. Examples of a liquid component that are generally inert in a Friedel-Crafts reaction include benzene, carbon disulfide, 1,2-dichloroethane, nitrobenzene, tetrachloroethane, tetrachloromethane, or a combination thereof. An example of a liquid component that is generally inert in a Grignard reaction includes diethyl ether. Examples of a liquid component that are generally inert in a halogenation reaction include dichlorobenzene, glacial acetic acid, nitrobenzene, tetrachloroethane, tetrachloromethane, trichlorobenzene, or a combination thereof. Examples of a liquid component that are generally inert in a hydrogenation reaction include an alcohol, dioxane, a hydrocarbon, glacial acetic acid, or a combination thereof. Examples of a liquid component that are generally inert in a ketene condensation reaction include acetone, benzene, diethyl ether, xylene, or a combination thereof. Examples of a liquid component that are generally inert in a nitration reaction include dichlorobenzene, glacial acetic acid, nitrobenzene, or a combination thereof. Examples of a liquid component that are generally inert in an oxidation reaction include glacial acetic acid, nitrobenzene, pyridine, or a combination thereof. Examples of a liquid component that are generally inert in a sulfonation reaction include dioxane, nitrobenzene, or a combination thereof.

A solvent-borne coating is a coating wherein 50% to 100%, the including all intermediate ranges and combinations thereof, of a coating's liquid component is not water. Generally, the liquid component of a solvent-borne coating comprises an organic compound, an inorganic compound, or a combination thereof. The liquid component of a solvent-borne coating may function as a solvent, a thinner, a diluent, a plasticizer, or a combination thereof. In certain embodiments, a solvent-borne coating may comprise water. In specific aspects, the water may function as a solvent, a thinner, a diluent, or a combination thereof. The water component of a solvent-borne coating may comprise 0% to 49.999%, the including all intermediate ranges and combinations thereof, of the liquid component. In certain embodiments, the water component of a water-borne coating may be fully or partly miscible in the non-aqueous liquid component. Examples of the percent of water that is miscible, by weight at 20° C., in various liquids typically used in solvent-borne coatings include 0.01% water in tetrachloroethylene; 0.02% water in ethylbenzene; 0.02% water in p-xylene; 0.02% water in tricholorethylene; 0.05% water in 1,1,1-tricholoroethane; 0.05% water in toluene; 0.1% water in hexane; 0.16% water in methylene chloride; 0.2% water in dibutyl ether; 0.2% water in tetrahydronaphthalene; 0.42% water in diisobutyl ketone; 0.5% water in cyclohexyl acetate; 0.5% water in nitropropane; 0.6% water in 2-nitropropane; 0.62% water in butyl acetate; 0.72% water in dipentene; 0.9% water in nitroethane; 1.2% water in diethyl ether; 1.3% water in methyl tert-butyl ether; 1.4% water in trimethylcyclohexanone; 1.65% water in isobutyl acetate; 1.7% water in butyl glycol acetate; 1.9% water in isopropyl acetate; 2.4% water in methyl isobutyl ketone; 3.3% water in ethyl acetate; 3.6% water in cyclohexanol; 4.0% water in trimethylcyclohexanol; 4.3% water in isophorone; 5.8% water in methylbenzyl alcohol; 6.5% water in ethyl glycol acetate; 7.2% water in hexanol; 7.5% water in propylene carbonate; 8.0% water in methyl acetate; 8.0% water in cyclohexanone; 12.0% water in methyl ethyl ketone; 16.2% water in isobutanol; 19.7% water in butanol; 25.0% water in butyl glycolate; or 44.1% water in 2-butanol.

Various examples of such liquid components are described herein, including properties often used to select a chemical composition for use as a liquid component for a particular coating composition. Additionally, standards for physical properties, chemical properties, and/or procedures for testing purity/properties, are described for various types of liquid components (e.g., hydrocarbons, cycloaliphatic hydrocarbons, aromatic hydrocarbons, alcohols, ketones, esters, glycol ethers, mineral spirits, miscellaneous solvents, plasticizers) in, for example, “ASTM Book of Standards, Volume 06.04, Paint—Solvents; Aromatic Hydrocarbons,” D4790-99, D268-01, D3437-99, D1493-97, D235-02, D1836-02, D3735-02, D3054-98, D5309-02, D4734-98, D2359-02, D4492-98, D4077-00, D3760-02, D6526-00, D841-02, D843-97, D5211-01, D5471-97, D5871-98, D5713-00, D852-02, D1685-00, D4735-02, D3797-00, D3798-00, D5135-02, D5136-00, D5060-95, D3193-96, D3734-01, D1152-97, D770-95, D3622-95, D1007-00, D1719-95, D304-95, D319-95, D2635-01, D1969-01, D2306-00, D1612-95, D5008-01, D268-01, D1078-01, D329-02, D1363-94, D740-94, D2804-02, D1153-94, D3329-99, D2917-02, D3893-99, D4360-90, D2627-02, D2916-88, D2192-96, D4614-95, D3545-02, D3131-02, D3130-95, D1718-98, D4615-95, D3540-90, D1617-90, D2634-02, D5137-01, D3728-99, D4835-93, D4773-02, D3128-02, D331-95, D330-93, D4837-02, D4773-02, D4836-95, D5776-99, D5808-95, D5917-02, D6069-01, D6212-99, D6313-99, D6366-99, D6428-99, D6621-00, D6809-02, D5399-95, D6229-01, D6563-00, D6269-98, D3257-01, D847-96, D1613-02, D848-02, D1614-95, D4367-02, D4534-99, D2360-00, D1353-02, D1492-02, D849-02, D3961-98, D1364-02, D3160-96, D1476-02 and D1722-98, D853-97, D5194-96, D363-90, D1399-95, D1468-93, D3620-98, D3546-90, and D1721-97, 2002.

a. Solvents, Thinners, and Diluents

A coating may comprise a liquid component that may function as a solvent, a thinner, a diluent or a combination thereof. In one embodiment of a coating, a particular liquid component may function as a solvent, while in another coating composition comprising, for example, a different binder the same liquid component may function as a thinner and/or a diluent. Whether a liquid component functions primarily as a solvent, a thinner, or a diluent depends considerably upon the particular solvent and/or Theological property the liquid component confers to a specific coating composition. For example, the ability of the liquid component to function as a solvent, or lack thereof of such ability, relative to the other coating components generally differentiates a solvent from a diluent. A thinner is primarily included into a coating composition in combination with a solvent and/or diluent to alter a Theological property such as to reduce viscosity, enhance flow, enhance leveling, or a combination thereof. In addition to the additional techniques in the art to discern such differences of use for a specific liquid composition in a coating, examples of differing solubility properties for specific categories of liquid components, and empirical techniques for determining the solubility properties of a specific liquid component, relative to another coating component, are described herein.

A solute is a coating component dissolved by a solvent liquid component. A solute may be in solid, liquid or gas from prior to being dissolved. Solvency (“solvent power”) is the ability of a solvent to dissolve a solute, maintain a solute in solution upon addition of a diluent, and reduce the viscosity of a solution. A solvent is typically used to produce a solvent-borne coating, wherein the coating possesses desirable a rheological property for application to a surface and/or creation of a film of a desirable thickness. Additionally, a solvent may contribute to an appearance property, a physical property, a chemical property, or a combination thereof, of a coating and/or film. In many embodiments, a solvent is a volatile component of a coating, wherein 50% to 100%, including all intermediate ranges and combinations thereof, of the solvent is lost (e.g., evaporates) during film formation. In certain aspects, the rate of solvent loss slows during application and/or film formation. Such a change in solvent loss rate may promote a desirable rheologically related property during application and/or initial film formation, such as ease of application, minimum sag, reduce excessive flow, or a combination thereof, while still promoting a desirable rheologically related property post-application, such as a desirable leveling property, a desirable adhesion property, or a combination thereof.

Depending upon the ability of a liquid component to dissolve, partly dissolve, or unsuccessfully dissolve a coating component, a coating may comprise, a real solution, a colloidal solution or a dispersion, respectively. Often the ability of a liquid component to dissolve a coating component is detrimentally affected by increasing particulate matter size (e.g., pigment size, cell-based particulate material size, etc.) and/or molecular mass of the coating component. For example, a real solution comprises a clear and/or homogenous liquid solution. In typical embodiments, a real solution is produced when a potential solute of 1.0 nm or less in diameter is combined with a solvent. A colloidal solution comprises a physically non-homogenous solution, which may be a clear to opalescent in appearance. Often, a colloidal solution is produced when a potential solute of between 1.0 nm to 100 nm (“0.1 μm”) in diameter is combined with a solvent. A dispersion is a composition comprising two liquid and/or solid phases, which is typically turbid to milky in appearance. Generally, a dispersion is produced when a potential solute of greater than 0.1 μm in diameter is combined with a solvent. In many aspects, a coating composition may comprise a combination of a real solution, a colloidal solution and/or a dispersion, depending upon the various solubility's of coating components and liquid components. For example, a paint may comprise a real solution of a binder and a liquid component, and a dispersion of a pigment within the liquid component.

Depending upon other coating components, a liquid component may function as an active solvent or a latent solvent. An active solvent is capable of dissolving a solute. Additionally, an active solvent often reduces viscosity of a coating composition. In certain embodiments, an ester, a glycol ether, a ketone, or a combination thereof may be selected for use as an active solvent. A latent solvent, in pure form, does not demonstrate solute dissolving ability. However, the latent solvent may demonstrate the ability to dissolve a solute in a combination of an active solvent and the latent solvent; confer a synergistic improvement in the dissolving ability of an active solvent when combined with the active solvent, or a combination thereof. In certain embodiments, an alcohol may be selected for use as a latent solvent. In certain embodiments, a latent solvent is a thinner. A diluent, whether in pure form or in combination with an active solvent and/or a latent solvent, does not demonstrate solute dissolving ability, but may be combined with an active solvent and/or latent solvent to produce a liquid component with a suitable ability to dissolve a coating component. In certain embodiments, hydrocarbon may be selected for use as a diluent. In particular aspects, a hydrocarbon diluent comprises an aromatic hydrocarbon, an aliphatic hydrocarbon, or a combination thereof. In particular facets, an aromatic hydrocarbon diluent may be selected, due to a generally greater tolerance by a many solvents relative to an aliphatic hydrocarbon. In certain aspects, a diluent is used to alter a rheological property (e.g., reduce viscosity) of a coating composition, reduce cost of a coating composition, or a combination thereof.

The ability of a solvent to dissolve a potential solute is related to the intermolecular interactions between the solvent molecules, between the potential solute molecules, between the solvent and the potential solute, as well as the molecular size of the potential solute. Examples of intermolecular interactions include, for example, ionic (“Coulomb”), dipole-dipole (“directional”), ionic-dipole, induction (“permanent dipole/induced dipole”), dispersion (“nonpolar,” “atomic dipole,” “London-Van der Walls”), hydrogen bond, or a combination thereof. The sum of intramolecular interactions for a compound, relevant for the preparation of a solution, is the solubility parameter (“δ”). The solubility parameter is a measure of the total energy needed to separate molecules of a liquid. Such a separation of molecules of a solvent occurs during the incorporation of the molecules of a solute during the dissolving process. The solubility parameter is the square root of the molar energy of vaporization of a liquid divided by the molar volume of a liquid, measured at 25° C. Additionally, the solubility parameter can also be expressed as the square root of the sum of the squares of the dispersion (“δ_{d}”), polar (“δₚ”) and hydrogen bond (“δₕ”) solubility parameters.

Often, preparation of a coating composition may be aided by comparing the solubility parameter of a potential solvent and a potential solute (e.g., a binder) to ascertain the theoretical ability of a coating composition comprising a solution to be created. In many embodiments, coating components, wherein at least one coating component comprises a liquid, with a solubility parameter that is less than an absolute value of 6 are able to form a solution. The closer this value is to 0, the greater the general ability to form a solution. Additionally, the lower the individual absolute difference (e.g., six or less) between the dispersion solubility parameters of coating components, the polar solubility parameter of coating components, and/or the hydrogen bond solubility parameter of coating components, the generally greater ability to form a solution. The solubility parameter, dispersion solubility parameter, polar solubility parameter, and hydrogen bond solubility parameter, and methods for determining such values, and additional methods for determining the theoretical ability of coating components to form a solution have been described (see, for example, in “ASTM Book of Standards, Volume 06.03, Paint—Pigments, Drying Oils, Polymers, Resins, Naval Stores, Cellulosic Esters, and Ink Vehicles,” D3132-84, 2002).

However, due to exceptions to the ability of certain liquid components and potential solute coating components to form solutions, empirically determining the ability of a solute to dissolve in a solvent may be desirable in certain embodiments. Standard techniques in the art may be used for determining the ability of a liquid component comprising one or more liquids to function as an active solvent, a latent solvent, a diluent, or a combination thereof, relative to one or more potential solutes. For example, the solvency of a liquid component comprising an active solvent (e.g., an oxygenated compound), a latent solvent, a diluent (e.g., a hydrocarbon), or a combination thereof, particularly for use in a lacquer coating, may be determined as described in “ASTM Book of Standards, Volume 06.04, Paint—Solvents; Aromatic Hydrocarbons,” D1720-96, 2002). In an additional example, the solvency for a liquid component that primarily comprises a hydrocarbon, and comprises little or lacks an oxygenated compound, may be determined as described in “ASTM Book of Standards, Volume 06.04, Paint—Solvents; Aromatic Hydrocarbons,” D1133-02, 2002). In a further example, the solvency of a solution comprising a liquid component and an additional coating component (e.g., a binder) may be determined, as described in “ASTM Book of Standards, Volume 06.03, Paint—Pigments, Drying Oils, Polymers, Resins, Naval Stores, Cellulosic Esters, and Ink Vehicles,” D1545-98, D1725-62, D5661-95, D5180-93, D6038-96, D5165-93, and D5166-97, 2002. In a supplemental example, the dilutability of a solution comprising liquid component (e.g., a solvent and diluent) and an additional coating component (e.g., a binder) may be determined, as described in “ASTM Book of Standards, Volume 06.03, Paint—Pigments, Drying Oils, Polymers, Resins, Naval Stores, Cellulosic Esters, and Ink Vehicles,” D5062-96, 2002.

In certain embodiments, a liquid component may be selected on the basis of evaporation rate. The evaporation rate of a coating directly affects a physical aspect of film formation caused by loss of a liquid component, as well as the pot life of a coating, such as after a coating container is opened. Though the evaporation rate is known for various pure chemicals, empirical determination of the evaporation rate of a liquid component and/or a coating may be done, as described, for example, in “ASTM Book of Standards, Volume 06.01, Paint—Tests for Chemical, Physical, and Optical Properties; Appearance,” D3539-87, 2002. Additionally, the boiling point range of a liquid component often is useful in estimating whether the liquid component will evaporate faster or slower relative to another liquid component. Examples of methods for measuring a boiling point for a liquid component (e.g., a hydrocarbon, a chlorinated hydrocarbon) are described in “ASTM Book of Standards, Volume 06.04, Paint—Solvents; Aromatic Hydrocarbons,” D1078-01 and D850-02e1, 2002. The evaporation rate is also related to the flash point of a liquid component and/or coating. In certain embodiments, a liquid component may be selected on the basis of flash point and/or fire point, which is a measure of the danger of use of a flammable coating composition in, for example, storage, application in an indoor environment, etc. A flash point is the “lowest temperature at which the liquid gives off enough vapor to form an ignitable mixture with air to produce a flame when a source of ignition is brought close to the surface of the liquid under specified conditions of test at standard barometric pressure (760 mmHG, 101.3 kPa),” and a fire point is “the lowest temperature at which sustained burning of the sample takes place for at least 5 seconds” [“Paint and Coating Testing Manual, Fourteenth Edition of the Gardner-Sward Handbook” (Koleske, J. V. Ed.), pp. 140 and 142, 1995]. Examples of methods for measuring the flash point and/or fire point for a liquid component and/or a coating are described in and “ASTM Book of Standards, Volume 06.01, Paint—Tests for Chemical, Physical, and Optical Properties; Appearance,” D1310-01, D3934-90, D3941-90, and D3278-96e1, 2002.

Though it is contemplated that much or all liquid components will be lost from a coating composition during film formation, a liquid component may still contribute to the visual properties of a coating and/or film. In embodiments wherein a liquid component is selected as a colorizing agent, the color and/or darkness of the liquid may be empirically measured (see, for example, “ASTM Book of Standards, Volume 06.04, Paint—Solvents; Aromatic Hydrocarbons,” D1209-00, D1686-96, and D5386-93b, 2002); and “ASTM Book of Standards, Volume 06.01, Paint—Tests for Chemical, Physical, and Optical Properties; Appearance,” D1544-98, 2002. In some embodiments, a liquid component and/or coating may be selected on the basis of odor (e.g., faint odor, pleasant odor, etc.). A coating or coating component can be evaluated for suitability in a particular application based on odor using, for example, techniques described in “ASTM Book of Standards, Volume 06.04, Paint—Solvents; Aromatic Hydrocarbons,” D1296-01, 2002; and “ASTM Book of Standards, Volume 06.01, Paint—Tests for Chemical, Physical, and Optical Properties; Appearance,” D6165-97, 2002.

(1) Hydrocarbons

A hydrocarbon is typically obtained as a petroleum product, a vegetable product, or a combination thereof. As a consequence of imperfect purification (e.g., distillation) from these sources, a hydrocarbon is often a mixture of chemical components. A hydrocarbon may be selected as an active solvent to dissolve an oil (e.g., a drying oil), an alkyd, an asphalt, a rosin, a petroleum product, or a combination thereof. A hydrocarbon is more suitable as a latent solvent or diluent in embodiments wherein an acrylic resin, an epoxide resin, a nitrocellulose resin, a urethane resin, or a combination thereof is to be dissolved. However, a hydrocarbon generally is immiscible in water.

(i) Aliphatic Hydrocarbons

In general embodiments, an aliphatic hydrocarbon may be selected as an active solvent for an alkyd, an oil, wax, a polyisobutene, a polyethylene, a poly(butyl acrylate), a poly(butyl methacrylate), a poly(vinyl ethers), or a combination thereof. In other embodiments, an aliphatic hydrocarbon may be selected as a diluent in combination with an additional liquid component. In alternative embodiments wherein an aliphatic hydrocarbon is selected as a non-solvent liquid component, a composition comprising a polar binder, a cellulose derivative, or a combination thereof, is usually insoluble. An aliphatic hydrocarbon is often selected as a liquid component in embodiments wherein a chemically inert liquid component is desired. Examples of an aliphatic hydrocarbon include, a petroleum ether, pentane (CAS No. 109-66-0), hexane (CAS No. 110-54-3), heptane (CAS No. 142-82-5), isododecane (CAS No. 13475-82-6), a kerosene, a mineral spirit, a VMP naphthas or a combination thereof. A hexane, a heptane, or a combination thereof, may be selected for a coating wherein rapid evaporation of such a liquid component is desired (e.g., a fast drying lacquer). An example of an azeotrope comprising an aliphatic hydrocarbon includes an azeotrope comprising hexane. Examples of an azeotrope comprising a majority of hexane (BP 65° C. to 70° C.) include those comprising 2.5% isobutanol (azeotrope BP 68.3° C.); 5.6% water (A-BP 61.6° C.); 21% ethanol (A-BP 58.7° C.); 22% isopropyl alcohol (A-BP 61.0° C.); 26.9% methanol (A-BP 50.0° C.); 37% methyl ethyl ketone (A-BP 64.2° C.); or 42% ethyl acetate (A-BP 65.0° C.).

An aliphatic hydrocarbon can comprise a petroleum distillation product of heterogeneous chemical composition. Such an aliphatic hydrocarbon may be classified by a physical and/or chemical property (e.g., boiling point range, flash point, evaporation rate) (see, for example, “ASTM Book of Standards, Volume 06.04, Paint—Solvents; Aromatic Hydrocarbons,” D235-02 and D3735, 2002). In certain embodiments, such a petroleum distillation product aliphatic hydrocarbon may be classified, for example, as a mineral spirit, a VMP naphthas or a kerosene (e.g., deodorized kerosene). A mineral spirit (“white spirit,” “petroleum spirit”) is a petroleum distillation fraction with a boiling point between 149° C. to 204° C., including all intermediate ranges and combinations thereof, and a flash point of 38° C. or greater. A mineral spirit may further be classified as a regular mineral spirit, which possesses the properties previously described for a mineral spirit; a high flash mineral spirit, which possesses a higher minimum flash point (e.g., 55° C. or greater); a low dry point mineral spirit (“Stoddard solvent”), which typically evaporates 50% faster than a regular mineral spirit; or an odorless mineral spirit, which generally possesses less odor than a regular mineral spirit, but may also possess relatively weaker solvency property. A mineral spirit may be selected for embodiments wherein a solvent and/or diluent is desired for an alkyd coating, a chlorinated rubber coating, an oil-coating, a vinyl chloride copolymer coating, or a combination thereof. A VMP naphtha possess a similar solvency property as a mineral spirit, but evaporates faster with a BP of 121° C. to 149° C., including all intermediate ranges and combinations thereof, and typically has a flash point of 4° C. or greater. A VMP naphtha may further be classified as a regular VMP naphtha, which possesses the properties previously described for a VMP naphtha; a high flash VMP naphtha, which possesses a higher minimum flash point (e.g., 34° C. or greater); or an odorless VMP naphtha, which generally possesses less odor than a regular mineral spirit. A VMP naphtha may be selected for a coating that is spray applied, an industrial coating, or a combination thereof. A petroleum ether is a petroleum distillation fraction with a boiling point between 35° C. to 80° C., including all intermediate ranges and combinations thereof, with a low flash point (e.g., −46° C.), and may be used in embodiments wherein rapid evaporation is desired.

(ii) Cycloaliphatic Hydrocarbons

In embodiments wherein a cycloaliphatic hydrocarbon is selected as a solvent, a composition comprising an oil, alkyd, bitumen, rubber, or a combination thereof, usually can be dissolved. In alternative embodiments wherein a cycloaliphatic hydrocarbon is selected as a non-solvent liquid component, a composition comprising a polar binder such as a urea-formaldehyde binder, a melamine-formaldehyde binder, a phenol-formaldehyde binder; a cellulose derivative, such as, a cellulose ester binder; or a combination thereof, is usually insoluble. A cycloaliphatic hydrocarbon is generally soluble in other organic solvents, but not soluble in water. Examples of a cycloaliphatic hydrocarbon include cyclohexane (CAS No. 110-82-7); methylcyclohexane (CAS No. 108-87-2); ethylcyclohexane (CAS No. 1678-91-7); tetrahydronaphthalene (CAS No. 119-64-2); decahydronaphthalene (CAS No. 91-17-8); or a combination thereof. Tetrahydronaphthalene is often selected for coatings wherein oxidation of a binder is often occurs during film formation; a high gloss is typically occurs in a film, smooth surface is generally aproperty in a film, or a combination thereof. An example of an azeotrope comprising a cycloaliphatic hydrocarbon includes an azeotrope comprising cyclohexane. Examples of an azeotrope comprising a majority of cyclohexane (BP 80.5° C. to 81.5° C.) include those comprising 8.5% water (A-BP 69.8° C.); 10% butanol (A-BP 79.8° C.); 14% isobutanol (A-BP 78.1° C.); 20% propanol (A-BP 74.3° C.); 37% methanol (A-BP 54.2° C.); or 40% methyl ethyl ketone (A-BP 72.0° C.).

(iii) Terpene Hydrocarbons

A terpene typically possesses a superior solvency property, stronger odor, or a combination thereof, relative to an aliphatic hydrocarbon. Examples of a terpene include wood terpentine oil (CAS No. 8008-64-2); pine oil (CAS No. 8000-41-7); α-pinene (CAS No. 80-56-8); β-pinene; dipentene (CAS No. 138-86-3); D-limonene (CAS No. 5989-27-5); or a combination thereof. Dipentene may be selected for embodiments wherein a superior solvency property, a slower evaporation rate, or a combination thereof, relative to a turpentine, is desired. Pine oil may be classified as an oxygenated compound, but is described under hydrocarbons due to convention by those of skill in the art. Pine oil generally comprises a terpene alcohol. Pine oil may be selected for embodiments wherein a greater range of solvency for solutes, a slow evaporation rate, or a combination thereof, is desired. An example of an azeotrope comprising a terpene includes an azeotrope comprising α-pinene. An example of an azeotrope comprising a majority of α-pinene (BP 154.0° C. to 156.0° C.) includes an azeotrope comprising 35.5% cyclohexanol (A-BP 149.9° C.).

A terpene hydrocarbon (“terpene”) can comprise a by-product from pines tree and/or citrus processing of heterogeneous chemical composition. Such a terpene hydrocarbon (e.g., a terpentine) may be classified by a physical and/or chemical property (see, for example, “ASTM Book of Standards, Volume 06.03, Paint—Pigments, Drying Oils, Polymers, Resins, Naval Stores, Cellulosic Esters, and Ink Vehicles,” D804-02, D13-02, D233-02, D801-02, D802-02, and D6387-99, 2002. Examples of a terpentine include a gum turpentine, a steam-distilled wood turpentine, a sulfate wood turpentine, a destructively distilled wood turpentine, or a combination thereof. Both a gum turpentine and a sulfate wood turpentine generally comprise a combination of α-pinene and a lesser quantity of β-pinene. A steam-distilled wood terpentine generally comprises α-pinene and a lesser component of dipentene and one or more other terpenes. Destructively distilled wood turpentine generally comprises various aromatic hydrocarbons and a lesser quantity of one or more terpenes.

(iv) Aromatic Hydrocarbons

An aromatic hydrocarbon typically possesses a greater solvency property and/or odor relative to other hydrocarbon types. Examples of an aromatic hydrocarbon include benzene (CAS No. 71-43-2); toluene (CAS No. 108-88-3; “methylbenzene”); ethylbenzene (CAS No. 100-41-4); xylene (CAS No. 1330-20-7); cumene (“isopropylbenzene”; CAS No. 98-82-8); a type I high flash aromatic naphthas; a type II high flash aromatic naphthas; mesitylene (CAS No. 108-67-8); pseudocumene (CAS No. 95-63-6); cymol (CAS No. 99-87-6); styrene (CAS No. 100-42-5); or a combination thereof. Xylene typically comprises o-xylene (CAS No. 56004-61-6); m-xylene (CAS No. 108-38-3); p-xylene (CAS No. 41051-88-1); and trace ethylbenzene. Toluene may be selected for embodiments wherein rapid evaporation is desired. In specific aspects, toluene may be selected for a spray applied coating, an industrial coating, or a combination thereof. Xylene may be selected for embodiments wherein a moderate evaporation rate is desired. In specific aspects, xylene may be selected for an industrial coating. An aromatic hydrocarbon may comprise a petroleum-processing product of heterogeneous chemical composition such as a high flash aromatic naphtha (e.g., type I, type II). A type I high flash aromatic naphtha and type II high flash aromatic naphtha possess a minimum flash point of 38° C. and 60° C., respectively. Standards for the characteristic chemical an/or physical property of an aromatic naphtha have been described (see, for example, “ASTM Book of Standards, Volume 06.04, Paint—Solvents; Aromatic Hydrocarbons,” D3734, 2002). A high flash naphtha typically has a slow evaporation rate. In specific embodiments, a high flash aromatic naphtha may be used in an industrial coating, a coating that is baked, or a combination thereof. An example of a high flash aromatic is Solvesso 100 (CAS No. 64742-95-6). Examples of an azeotrope comprising an aromatic hydrocarbon include an azeotrope comprising toluene or m-xylene. Examples of an azeotrope comprising a majority of toluene (BP 110° C. to 111° C.) include those comprising 27% butanol (A-BP 105.6° C.); or 44.5% isobutanol (A-BP 100.9° C.). Examples of an azeotrope comprising a majority of m-xylene (BP 137.0° C. to 142.0° C.) include those comprising 14% cyclohexanol (A-BP 143.0° C.); or 40% water (A-BP 94.5° C.).

(2) Oxygenated Compounds

An oxygenated compound (“oxygenated liquid compound”) is typically chemically synthesized by standard chemical manufacturing techniques. As a consequence, an individual oxygenated compound is typically an extremely homogenous chemical composition, with singular, rather than a range of, chemical and physical properties. The oxygen moiety of an oxygenated compound generally enhances the strength and breadth of solvency for potential solutes relative to a hydrocarbon. Additionally, an oxygenated compound typically has some or complete miscibility with water. Examples of an oxygenated compound include an alcohol, an ester, a glycol ether, a ketone, or a combination thereof. A liquid component often comprises a combination of an alcohol, an ester, a glycol ether, a ketone and/or an additional liquid to produce suitable chemical and/or physical properties for a coating and/or film.

(i) Alcohols

An alcohol comprises an alcohol moiety. However, a typical “alcohol” comprises a single hydroxyl moiety. The alcohol moiety confers miscibility with water. Consequentially, increasing molecular size of an alcohol comprising a single alcohol moiety generally reduces miscibility with water. Alcohols typically possess a mild and/or pleasant odor. An alcohol is typically a poor primary solvent, though ethanol is an exception relative to a solute comprising a phenolic and/or polyvinyl resin. An alcohol may be selected as a latent solvent, co-solvent, a coupling solvent, a diluent, or combination thereof such as with solute comprising a nitrocellulose lacquer, melamine-formaldehyde, urea formaldehyde, alkyd, or combination thereof. Examples of an alcohol include methanol (CAS No. 67-56-1); ethanol (CAS No. 64-17-5); propanol (CAS No. 71-23-8); isopropanol (CAS No. 67-63-0); 1-butanol (CAS No. 71-36-3); isobutanol (CAS No. 78-83-1); 2-butanol (CAS No. 78-92-2); tert-butanol (CAS No. 75-65-0); amyl alcohol (CAS No. 71-41-0); isoamyl alcohol (123-51-3); hexanol (25917-35-5); methylisobutylcarbinol (CAS No. 108-11-2); 2-ethylbutanol (CAS No. 97-95-0); isooctyl alcohol (CAS No. 26952-21-6); 2-ethylhexanol (CAS No. 104-76-7); isodecanol (CAS No. 25339-17-7); cylcohexanol (CAS No. 108-93-0); methylcyclohexanol (CAS No. 583-59-5); trimethylcyclohexanol; benzyl alcohol (CAS No. 100-51-6); methylbenzyl alcohol (CAS No. 98-85-1); furfuryl alcohol (CAS No. 98-00-0); tetrahydrofurfuryl alcohol (CAS No. 97-99-4); diacetone alcohol (CAS No. 123-42-2); trimethylcyclohexanol (116-02-9); or a combination thereof. Furfuryl alcohol and tetrahydrofurfuryl alcohol may be selected as a primary solvent for a polyvinyl binder. Examples of an azeotrope comprising an alcohol include an azeotrope comprising butanol, ethanol, isobutanol, or methanol. Examples of an azeotrope comprising a majority of butanol (BP 117.7° C.) include those comprising 97% butanol and 3% hexane (A-BP 67° C.); 32% p-xylene (A-BP 115.7° C.); 32.8% butyl acetate (A-BP 117.6° C.); 44.5% water (A-BP 93° C.); or 50% isobutyl acetate (A-BP 114.5° C.). Examples of an azeotrope comprising a majority of ethanol (BP 78.3° C.) include those comprising 4.4% water (A-BP 78.2° C.); or 32% toluene (A-BP 76.7° C.). Examples of an azeotrope comprising a majority of isobutanol (BP 107.7° C.) include those comprising 2.5% hexane (A-BP 68.3° C.); 5% isobutyl acetate (A-BP 107.6° C.); 17% p-xylene (A-BP 107.5° C.); 33.2% water (A-BP 89.9° C.); or 48% butyl acetate (A-BP 80.1° C.). An example of an azeotrope comprising a majority of methanol (BP 64.6° C.) includes an azeotrope comprising 30% methyl ethyl ketone (A-BP 63.5° C.).

(ii) Ketones

A ketone comprises a ketone moiety. However, a typical ketone comprises a single ketone moiety. A ketone generally possesses some miscibility with water, and a strong odor. In general embodiments, a ketone may be selected as a primary solvent, thinner, or combination thereof. Examples of a ketone include acetone (CAS No. 67-64-1); methyl ethyl ketone (CAS No. 78-93-3); methyl propyl ketone (CAS No. 107-87-9); methyl isopropyl ketone (CAS No. 563-80-4); methyl butyl ketone (CAS No. 591-78-6); methyl isobutyl ketone (CAS No. 108-10-1); methyl amyl ketone (CAS No. 110-43-0); methyl isoamyl ketone (CAS No. 110-12-3); diethyl ketone (CAS No. 96-22-0); ethyl amyl ketone (CAS No. 541-85-5); dipropyl ketone (CAS No. 110-43-0); diisopropyl ketone (CAS No. 565-80-0); cyclohexanone (CAS No. 108-94-1); methylcylcohexanone (CAS No. 1331-22-2); trimethylcyclohexanone (CAS No. 873-94-9); mesityl oxide (CAS No. 141-79-7); diisobutyl ketone (CAS No. 108-83-8); isophorone (CAS No. 78-59-1); or a combination thereof. Acetone may be selected for complete miscibility in water, fast evaporation, or a combination thereof. In certain embodiments, acetone may be used as a liquid component in an aerosol, a spray-applied coating, or a combination thereof. In specific aspects, acetone may be used as a thinner. In other aspects, acetone may be used in a coating wherein nitrocellulose, an acrylic, or a combination thereof, is dissolved. Methyl ethyl ketone, methyl isobutyl ketone, and isophorone may be selected in embodiments wherein a fast evaporation rate, moderate evaporation rate, or slow evaporation rate, respectively, is desired. In specific facets, isophorone may be selected for a baked coating, an industrial coating, or a combination thereof. Examples of an azeotrope comprising a ketone include an azeotrope comprising acetone, methyl ethyl ketone or methyl isobutyl ketone. Examples of an azeotrope comprising a majority of acetone (BP 56.2° C.) include those comprising 12% methanol (A-BP 55.7° C.); or 41% hexane (A-BP 49.8° C.). Examples of an azeotrope comprising a majority of methyl ethyl ketone (BP 79.6° C.) include those comprising 11% water (A-BP 73.5° C.); 32% isopropyl alcohol (A-BP 77.5° C.); or 34% ethanol (A-BP 74.8° C.). Examples of an azeotrope comprising a majority of methyl isobutyl ketone (BP 114° C. to 117° C.) include those comprising 24.3% water (A-BP 87.9° C.); or 30% butanol (A-BP 114.35° C.).

(iii) Esters

An ester may comprise an alkyl acetate, an alkyl propionate, a glycol ether acetate, or a combination thereof. An ester generally possesses a pleasant odor. In general embodiments, an ester possesses a solubility property that decreases with increasing molecular weight. A glycol ester acetate typically possesses a slow evaporation rate. In specific aspects, a glycol ester acetate may be selected as a retarder solvent, a coalescent, or a combination thereof. Examples of an ester include methyl formate (CAS No. 107-31-3); ethyl formate (CAS No. 109-94-4); butyl formate (CAS No. 592-84-7); isobutyl formate (CAS No. 542-55-2); methyl acetate (CAS No. 79-20-9); ethyl acetate (CAS No. 141-78-6); propyl acetate (CAS No. 109-60-4); isopropyl acetate (CAS No. 108-21-4); butyl acetate (CAS No. CAS-No. 123-86-4); isobutyl acetate (CAS No. 110-19-0); sec-butyl acetate (CAS No. 105-46-4); amyl acetate (CAS No. 628-63-7); isoamyl acetate (CAS No. 123-92-2); hexyl acetate (CAS No. 142-92-7); cyclohexyl acetate (CAS No. 622-45-7); benzyl acetate (CAS No. 140-11-4); methyl glycol acetate (CAS No. 110-49-6); ethyl glycol acetate (CAS No. 111-15-9); butyl glycol acetate (CAS No. 112-07-2); ethyl diglycol acetate (CAS No. 111-90-0); butyl diglycol acetate (CAS No. 124-17-4); 1-methoxypropyl acetate (CAS No. 108-65-6); ethoxypropyl acetate (CAS No. 54839-24-6); 3-methoxybutyl acetate (CAS No. 4435-53-4); ethyl 3-ethoxypropionate (CAS No. 763-69-9); isobutyl isobutyrate (CAS No. 97-85-8); ethyl lactate (CAS No. 97-64-3); butyl lactate (CAS No. 138-22-7); butyl glycolate (CAS No. 7397-62-8); dimethyl adipate (CAS No. 627-93-0); glutarate (CAS No. 119-40-0); succinate (CAS No. 106-65-0); ethylene carbonate (CAS No. 96-49-1); propylene carbonate (CAS No. 108-32-7); butyrolactone (CAS No. 96-48-0); or a combination thereof. Ethylene carbonate and propylene carbonate generally possess a high flash point, a slow evaporation rate, a weak odor, or a combination thereof. Ethylene carbonate is typically used for use in coatings at temperatures greater than 25° C. Examples of an azeotrope comprising an ester include an azeotrope comprising butyl acetate, ethyl acetate or methyl acetate. Examples of an azeotrope comprising a majority of butyl acetate (BP 124° C. to 128° C.) include those comprising 27% water (A-BP 90.7° C.) or 35.7% ethyl glycol (A-BP 125.8° C.). Examples of an azeotrope comprising a majority of ethyl acetate (BP 76° C. to 77° C.) include those comprising 5% cyclohexanol (A-BP 153.8° C.); 8.2% water (A-BP 70.4° C.); 22% methyl ethyl ketone (A-BP 76.7° C.); 23% isopropyl alcohol (A-BP 74.8° C.); or 31% ethanol (A-BP 71.8° C.). An example of an azeotrope comprising a majority of methyl acetate (BP 55.0° C.-57.0° C.) includes an azeotrope comprising 19% methanol (A-BP 54° C.).

(iv) Glycol Ethers

A glycol ether comprises an alcohol moiety and an ether moiety. The glycol ether generally possesses good solvency, high flash point, slow evaporation rate, mild odor, miscibility with water, or a combination thereof. In some embodiments, a glycol ether may be selected as a coupling solvent, a thinner, or a combination thereof. In particular aspects, a glycol ether may be selected as a liquid component of a lacquer. Examples of a glycol ether include methyl glycol (CAS No. 109-86-4); ethyl glycol (CAS No. 110-80-5); propyl glycol (CAS No. 2807-30-9); isopropyl glycol (CAS No. 109-59-1); butyl glycol (CAS No. 111-76-2); methyl diglycol (111-77-3); ethyl diglycol (CAS No. 111-90-0); butyl diglycol (CAS No. 112-34-5); ethyl triglycol (CAS No. 112-50-5); butyl triglycol (CAS No. 143-22-6); diethylene glycol dimethyl ether (CAS No. 111-96-6); methoxypropanol (CAS No. 107-98-2); isobutoxypropanol (CAS No. 23436-19-3); isobutyl glycol (CAS No. 4439-24-1); propylene glycol monoethyl ether (CAS No. 52125-53-8); 1-isopropoxy-2-propanol (CAS No. 3944-36-3); propylene glycol mono-n-propyl ether (CAS No. 30136-13-1); propylene glycol n-butyl ether (CAS No. 5131-66-8); methyl dipropylene glycol (CAS No. 34590-94-8); methoxybutanol (CAS No. 30677-36-2); or a combination thereof. An example of an azeotrope comprising a glycol ether includes an azeotrope comprising ethyl glycol. An example of an azeotrope comprising a majority of ethyl glycol (BP 134° C. to 137° C.) includes an azeotrope comprising 50% dibutyl ether (A-BP 127° C.).

(v) Ethers

Examples of an ether include diethyl ether (CAS No. 60-29-7); diisopropyl ether (CAS No. 108-20-3); dibutyl ether (CAS No. 142-96-1); di-sec-butyl ether (CAS No. 6863-58-7); methyl tert-butyl ether (CAS No. 1634-04-4); tetrahydrofuran (CAS No. 109-99-9); 1,4-dioxane (CAS No. 123-91-1); metadioxane (CAS No. 505-22-6); or a combination thereof. Tetrahydrofuran may be selected as a primary solvent for a polyvinyl binder. An example of an azeotrope comprising an ether includes an azeotrope comprising tetrahydrofuran. An example of an azeotrope comprising a majority of tetrahydrofuran (BP 66° C.) includes an azeotrope comprising 5.3% water (A-BP 64.0° C.).

(3) Chlorinated Hydrocarbons

A chlorinated hydrocarbon generally comprises a hydrocarbon, wherein the hydrocarbon comprises a chloride atom moiety. A chlorinated hydrocarbon generally possesses a high degree of non-flammability, and consequently lacks a flash point. A chlorinated hydrocarbon may be selected for embodiments where high flash point is desired. In particular facets, a chlorinated hydrocarbon may be added to a liquid component to reduce the liquid component's flash point. In certain facets, a chlorinated hydrocarbon may be combined with a mineral spirit, methylene chloride, or a combination thereof, for a reduction of the flash point. In particular aspects, a chlorinated hydrocarbon (e.g., methylene chloride, trichloroethylene) may be selected as a solvent for removal of hydrophobic material from a surface (e.g., grease, an undesired coating and/or film). However, a chlorinated hydrocarbon may be subject to an environmental regulation or law. Examples of a chlorinated hydrocarbon include methylene chloride (CAS No. 75-09-2; “dichloromethane”); trichloromethane (CAS No. 67-66-3); tetrachloromethane (CAS No. 56-23-5); ethyl chloride (CAS No. 75-00-3); isopropyl chloride (CAS No. 75-29-6); 1,2-dichloroethane (CAS No. 107-06-2); 1,1,1-trichloroethane (CAS No. 71-55-6; “methylchloroform”); trichloroethylene (CAS No. 79-01-6); 1,1,2,2-tetrachlorethane (CAS No. 79-55-6); 1,2-dichloroethylene (CAS No. 75-35-4); perchloroethylene (CAS No. 127-18-4); 1,2-dichloropropane (CAS No. 78-87-5); chlorobenzene (CAS No. 108-90-7); or a combination thereof. Methylene chloride may be selected for embodiments wherein a fast evaporation rate is desired. 1,1,1-trichloroethane may be selected for embodiments wherein a photochemically inert liquid component is desired. Additionally, methylene chloride may be selected as a coating remover. Examples of an azeotrope comprising a chlorinated hydrocarbon include an azeotrope comprising methylene chloride, trichloroethylene or 1,1,1-trichloroethane. Examples of an azeotrope comprising a majority of methylene chloride (BP 40.2° C.) include those comprising 1.5% water (A-BP 38.1° C.); 3.5% ethanol (A-BP 41.0° C.); or 8% methanol (A-BP 39.2° C.). Examples of an azeotrope comprising a majority of trichloroethylene (BP 86.7° C.) include those comprising 6.6% water (A-BP 72.9° C.); 27% ethanol (A-BP 70.9° C.); or 36% methanol (A-BP 60.2° C.). An example of an azeotrope comprising a majority of 1,1,1-trichloroethane (BP 74.0° C.) includes an azeotrope comprising 4.3% water (A-BP 65.0° C.).

(4) Nitrated Hydrocarbon

A nitrated hydrocarbon comprises a hydrocarbon, wherein the hydrocarbon comprises a nitrogen atom moiety. Examples of a nitrated hydrocarbon include a nitroparaffin, N-methyl-2-pyrrolidone (“NMP”), or a combination thereof. Examples of a nitroparaffin include nitroethane, nitromethane, nitropropane, 2-nitropropane (“2NP”), or a combination thereof. 2-nitropropane may be selected for embodiments as a substitute for butyl acetate relative to a solvent property, but wherein a greater evaporation rate is desired. N-methyl-2-pyrrolidone may be selected for embodiments wherein a strong solvent property, miscibility with water, high flash point, biodegradability, low toxicity, or a combination thereof is desired. In certain aspects, N-methyl-2-pyrrolidone may be used in a water-borne coating, a coating remover, or a combination thereof.

(5) Miscellaneous Organic Liquids

A miscellaneous organic liquid is a liquid comprising carbon that are useful as a liquid component for a coating, but are not readily classified as a hydrocarbon, an oxygenated compound, a chlorinated hydrocarbon, a nitrated hydrocarbon, or a combination thereof. Examples of a miscellaneous organic liquid include carbon dioxide; acetic acid, methylal (CAS No. 109-87-5); dimethylacetal (CAS No. 534-15-6); N,N-dimethylformamide (CAS No. 68-12-2); N,N-dimethylacetamide (CAS No. 127-19-5); dimethylsulfoxide (CAS No. 67-68-5); tetramethylene suflone (CAS No. 126-33-0); carbon disulfide (CAS No. 75-15-0); 2-nitropropane (CAS No. 79-46-9); N-methylpyrrolidone (CAS No. 872-50-4); hexamethylphosphoric triamide (CAS No. 680-31-9); 1,3-dimethyl-2-imidazolidinone (CAS No. 80-73-9); or a combination thereof. Carbon dioxide may function as a liquid component when prepared under pressure and temperature conditions to form a supercritical liquid. A supercritical liquid has properties between that of a liquid and a gas, and can be used in spray application of a coating wherein the appropriate pressure conditions can be maintained. Supercritical carbon dioxide may be formulated with a coating using the tradename technique Unicarb™ (Union Carbide Chemicals and Plastics Co., Inc.). Supercritical carbon dioxide may be selected as a substitute for a hydrocarbon diluent in embodiments wherein chemical inertness, non-flammability, rapid evaporation, or a combination thereof, is desirable. In certain aspects, 0% to 30%, including all intermediate ranges and combinations thereof, of a hydrocarbon liquid component may be replaced with supercritical carbon dioxide.

b. Plasticizers

In certain embodiments, a coating may comprise a plasticizer. A plasticizer may be selected for embodiments wherein the resin possesses an unsuitable brittleness and/or low flexibility property upon film formation. Properties a plasticizer typically confers to a coating and/or film include, for example, enhancing a flow property of a coating, lowering a film-forming temperature range, enhancing the adhesion property of a coating and/or film, enhancing the flexibility property of a film, lowering the T_{g}, improving film toughness, enhancing film heat resistance, enhancing film impact resistance, enhancing UV resistance, or a combination thereof. Since a function of a plasticizer typically is to alter a film's properties, many plasticizer's possess a high (e.g., baking temperature) boiling point, as such a compound is generally less volatile, with increasing boiling point temperature. In certain aspects, a plasticizer may function as a solvent, thinner, diluent, plasticizer, or combination thereof, for a coating composition and/or film at a temperature greater than ambient conditions.

A plasticizer is thought to interact with a binder by a polar interaction, but is chemically inert relative to the binder. A plasticizer typically will lower the T_{g} of a binder below the temperature a coating comprising the binder will be applied to a surface. In many embodiments, a plasticizer have a vapor pressure less than 3 mm at 200° C., a mass of 200 Da to 800 Da, a specific gravity of 0.75 to 1.35, a viscosity of 50 cSt to 450 cSt, a flash point temperature greater than 120° C., or a combination thereof. Certain plasticizers comprise an organic liquid (e.g., an ester). Standards for physical properties, chemical properties, and/or procedures for testing purity/properties, are described for plasticizers (e.g., undesired acidity, color, undesired copper corrosion, boiling point, ester content, odor, undesirable water contamination) in, for example, “ASTM Book of Standards, Volume 06.04, Paint—Solvents; Aromatic Hydrocarbons,” D1613-02, D1209-00, D849-02, D1078-01, D1617-90, D1296-01, D608-90, and D1364-02, 2002; and “ASTM Book of Standards, Volume 06.01, Paint—Tests for Chemical, Physical, and Optical Properties; Appearance,” D1544-98, 2002. Compatibility of a plasticizer with a binder and/or a solvent has been described (see, for example, Riley, H. E., “Plasticizers,” Paint Testing Manual, American Society for Testing Materials, 1972). Additionally, techniques previously described for estimating solubility for liquid and an additional coating component may be used for a plasticizer.

Various plasticizers comprise an ester of a monoalcohol and an acid (e.g., a dicarboxylic acid). In many embodiments, the monoalcohol comprises 4 to 13 carbons. In specific aspects, the monoalcohol comprises butanol, 2-ethylhexanol, isononanol, isooctyl, isodecyl, or a combination thereof. Examples of an acid include an azelaic acid, a phthalic acid, a sebacic acid, a trimellitic acid, an adipic acid, or a combination thereof. Examples of such plasticizers include di(2-ethylhexyl) azelate (“DOZ”); di(butyl) sebacate (“DBS”); di(2-ethylhexyl) phthalate (“DOP”); di(isononyl) phthalate (“DINP”); dibutyl phthalate (“DBP”); butyl benzyl phthalate (“BBP”); di(isooctyl) phthalate (“DIOP”); di(idodecyl) phthalate (“DIDP”); tris(2-ethylhexyl) trimellitate (“TOTM”); tris(isononyl) trimellitate (“TINTM”); di(2-ethylhexyl) adipate (“DOA”); di(isononyl) adipate (“DINA”); or a combination thereof.

A plasticizer may be classified by a moiety, such as, for example, as an adipate (e.g., DOA, DINA), an azelate (e.g., DOZ), a citrate, a chlorinated plasticizer, an epoxide, a phosphate, a sebacate (e.g., DBS), a phthalate (e.g., DOP, DINP, DIOP, DIDP), a polyester, or a trimellitate (e.g., TOTM, TINTM). An example of a citrate plasticizer includes acetyl tri-n-butyl citrate. Examples of an epoxide plasticizer include an epoxy modified soybean oil (“ESO”), 2-ethylhexyl epoxytallate (“2EH tallate”), or a combination thereof. Examples of a phosphate plasticizer include isodecyl diphenyl phosphate, tricresyl phosphate (“TPC”), isodecyl diphenyl phosphate, tri-2-ethylhexyl phosphate (“TOP”), or a combination thereof. Tricresyl phosphate may function as a plastizer, confer flame resistance, confer fungi resistance, or a combination thereof to a coating. Examples of a polyester plasticizer include an adipic acid polyester, an azelaic acid polyester, or a combination thereof. In certain aspects, a plasticizer is selected for water resistance (e.g., hydrolysis resistance, inertness toward water) such as a bisphenoxyethylformal.

c. Water-Borne Coatings

A water-borne coating (“water reducible coating”) refers to a coating wherein components such as a pigment, a binder, an additive, or a combination thereof are dispersed in water. Often, an additional solvent, surfactant, emulsifier, wetting agent, dispersant, or a combination thereof promotes dispersion of a coating component. A latex coating refers to a water-borne coating wherein the binder is dispersed in water. Typically, a binder of a latex coating comprises a high molecular weight binder. Often a latex coating (e.g., a paint, a lacquer) is a thermoplastic coating. Film formation occurs by loss of the liquid component, typically through evaporation, and fusion of dispersed thermoplastic binder particles. Often, a latex coating further comprises a coalescing solvent (e.g., diethylene glycol monobutyl ether) that promotes fusion of the binder particles. In some embodiments, a film produced from a latex coating is more porous, possesses a lower moisture resistance property, is less compact (e.g., thicker), or a combination thereof, relative to a solvent-borne coating comprising similar non-volatile components. Specific procedures for determining the purity/properties of a latex coating, coating component (e.g., solids content, nonvolatile content, vehicles), and/or film have been described, for example, in “ASTM Book of Standards, Volume 06.01, Paint—Tests for Chemical, Physical, and Optical Properties; Appearance,” D4747-02 and D4827-93, 2002; “ASTM Book of Standards, Volume 06.02, Paint—Products and Applications; Protective Coatings; Pipeline Coatings,” D3793-00, 2002; and “ASTM Book of Standards, Volume 06.03, Paint—Pigments, Drying Oils, Polymers, Resins, Naval Stores, Cellulosic Esters, and Ink Vehicles,” D5097-90 D4758-92, and D4143-89, 2002.

In certain embodiments, a water-borne coating is a coating wherein 50% to 100%, the including all intermediate ranges and combinations thereof, of a coating's liquid component is water. In general embodiments, the water component of a water-borne coating may function as a solvent, a thinner, a diluent, or a combination thereof. In certain embodiments, a water-borne coating may comprise an additional non-aqueous liquid component. In specific aspects, such an additional liquid component may function as a solvent, a thinner, a diluent, a plasticizer, or a combination thereof. An additional liquid component of a water-borne coating may comprise 0% to 49.999%, the including all intermediate ranges and combinations thereof, of the liquid component. Examples of additional liquid components in a water-borne coating include a glycol ether, an alcohol, or a combination thereof.

In certain embodiments, an additional liquid component of a water-borne coating may be fully or partly miscible in water. Examples of a liquid that is completely miscible in water, and visa versa, include methanol, ethanol, propanol, isopropyl alcohol, tert-butanol, ethylene glycol, methyl glycol, ethyl glycol, propyl glycol, butyl glycol, ethyl diglycol, methoxypropanol, methyldipropylene glycol, dioxane, tetrahydrorfuran, acetone, diacetone alcohol, dimethylformamide or dimethyl sulfoxide. Examples of a liquid that is partly miscible in water, by weight at 20° C., include 0.02% ethylbenzene; 0.02% tetrachloroethylene; 0.02% p-xylene; 0.035% toluene; 0.04% diisobutyl ketone; 0.1% tricholorethylene; 0.19% trimethylcyclohexanol; 0.2% cyclohexyl acetate; 0.3% dibutyl ether; 0.3% trimethylcyclohexanone; 0.44% 1,1,1-tricholoroethane; 0.53% hexane; 0.58% hexanol; 0.67% isobutyl acetate; 0.83% butyl acetate; 1.2% isophorone; 1.4% nitropropane; 1.5% butyl glycol acetate; 1.7% 2-nitropropane; 2.0% methylene chloride; 2.0% methyl isobutyl ketone; 2.3% cyclohexanone; 2.9% isopropyl acetate; 2.9% methylbenzyl alcohol; 3.6% cyclohexanol; 4.5% nitroethane; 4.8% methyl tert-butyl ether; 6.1% ethyl acetate; 6.9% diethyl ether; 7.5% butanol; 7.5% butyl glycolate; 8.4% isobutanol; 12.5% 2-butanol; 21.4% propylene carbonate; 23.5% ethyl glycol acetate; 24% methyl acetate; or 26.0% methyl ethyl ketone. Examples of an azeotrope comprising a majority of water (BP 100° C.) include those comprising 16.1% isophorone (A-BP 99.5° C.); 20% 2-ethylhexanol (A-BP 99.1° C.); 20% cyclohexanol (A-BP 97.8° C.); 20.8% butyl glycol (A-BP 98.8° C.); or 28.8% ethyl glycol (A-BP 99.4° C.).

3. Colorants

A colorant (“colorizing agent”) is a composition that confers a desirable optical property to a coating. Examples of desirable optical properties, depending upon the application, include a reflection property, a light absorption property, a light scattering property, or a combination thereof. A colorant that increases the reflection of light may increase gloss. A colorant that increased light scattering may increase the opacity and/or confer a color to a coating and/or film. Light scattering of a broad spectrum of wavelengths can confer a white color to a coating and/or film. Scattering of a certain wavelength may confer a color associated with the wavelength to a coating and/or film. Light absorption also affects opacity and/or color. Light absorption over a broad spectrum confers a black color to a coating and/or film. Absorbance of a certain wavelength may eliminate the color associated with the wavelength from the appearance of a coating and/or film. Examples of colorants include pigments, dyes, extenders, or a combination thereof. Colorants (e.g., pigments, dyes) and procedures for determining the optical properties and physical properties (e.g., hiding power, transparency, light absorption, light scattering, tinting strength, color, particle size, particle dispersion, pigment content, color matching) of a colorant, coating component, coating and/or film are described in, for example, (in “Industrial Color Testing, Fundamentals and Techniques, Second, Completely Revised Edition,” 1995; “Colorants for Non-Textile Applications,” 2000). Various colorants in the art may be used, and are often identified by their Colour Index (“CI”) number (see, for example, “Colour Index International,” 1971; and “Colour Index International,” 1997). In some cases, a common name for a colorant encompasses several related colorants, which can be differentiated by CI number.

a. Pigments

A pigment is a composition that is insoluble in the other components of a coating, and further confers a desirable optical properties, confers a property affecting the application of the coating (e.g., a rheological property), confers a performance property to a coating, reduces the cost of the coating, or a combination thereof. In certain embodiment, a pigment confers a performance property to a coating such as a desirable corrosion resistance property, magnetic property, or a combination thereof. Examples of a pigment include an inorganic pigment, an organic pigment, or a combination thereof.

Pigments possess a variety of properties in addition to color that aid in the selection of a particular pigment for a specific application. Examples of such properties include a tinctorial property, an insolubility property, a corrosion resistance property, a durability property, a heat resistance property, an opacity property, a transparency property, or a combination thereof. A tinctorial property is the ability of a composition to produce a color, wherein a greater tinctorial strength indicating less of the composition is needed to achieve the color. A insolubility property is the ability of a composition to remain in a solid form upon contact with another coating component (e.g., a liquid component), even during a curing process involving chemical reactions (e.g., thermosetting, baking, irradiation). A corrosion resistance property is the ability of a composition to reduce the damage of a chemical (e.g., water, acid) that contacts metal.

Pigments (e.g., extenders, titanium pigments, inorganic pigments, surface modified pigments, bismuth vanadates, cadmium pigments, cerium pigment, complex inorganic color pigments, metallic pigments, benzimidazolone pigments, diketopyrrolopyrrole pigments, dioxazine violet pigments, disazocondensation pigments, isoindoline pigments, isoindolinone pigments, perylene pigments, phthalocyanine pigments, quinacridone pigments, quinophthalone pigments, thiazine pigments, oxazine pigments, zinc sulfide pigments, zinc oxide pigments, iron oxide pigments, chromium oxide pigments, cadmium pigments, cadmium sulfide, cadmium yellow, cadmium sulfoselenide, cadmium mercury sulfide, bismuth pigments, chromate pigments, chrome yellow, molybdate red, molybdate orange, chrome orange, chrome green, fast chrome green, ultramarine pigments, iron blue pigments, black pigments, carbon black, specialty pigments, magnetic pigments, cobalt-containing iron oxide pigments, chromium dioxide pigments, metallic iron pigments, barium ferrite pigments, anti-corrosive pigments, phosphate pigments, zinc phosphate, aluminum phosphate, chromium phosphate, metal phosphates, multiphase phosphate pigments, borosilicate pigments, borate pigments, chromate pigments, molybdate pigments, lead cyanamide pigments, zinc cyanamide pigments, iron-exchange pigments, metal oxide pigments, red lead pigment, red lead, calcium plumbate, zinc ferrite pigments, calcium ferrite pigments, zinc oxide pigments, powdered metal pigments, zinc dust, lead powder, flake pigments, nacreous pigments, interference pigments, natural pearl essence pigment, basic lead carbonate pigment, bismuth oxychloride pigment, metal oxide-mica pigments, metal effect pigments, transparent pigments, transparent iron oxide pigments, transparent iron blue pigment, transparent cobalt blue pigment, transparent cobalt green pigment, transparent iron oxide, transparent zinc oxide, luminescent pigments, inorganic phosphor pigments, sulfide pigments, selenide pigments, oxysulfide pigments, oxygen dominant phosphor pigments, halide phosphor pigments, azo pigments, monoazo yellow pigments, monoazo orange pigment, disazo pigments, β-naphthol pigments, naphthol AS pigments, salt-type azo pigments, benzimidazolone pigments, disazo condensation pigments, metal complex pigments, isoindolinone pigments, isoindoline pigments, polycyclic pigments, phthalocyanine pigments, quinacrindone pigments, perylene pigments, perinone pigments, diketopyrrolo pyrrole pigments, thioindigo pigments, anthrapyrimidine pigments, flavanthrone pigments, pyranthrone pigments, anthanthrone pigments, dioxanzine pigments, triarylcarbonium pigments, quinophthalone pigments) and their chemical properties, physical properties and/or optical properties (e.g., color, tinting strength, lightening power, scattering power, hiding power, transparency, light stability, weathering resistance, heat stability, chemical fastness, interactions with a binder), in coating component, coating and/or film, and techniques for determining such properties, have been described (see, for example, Solomon, D. H. and Hawthorne, D. G., “Chemistry of Pigments and Fillers,” 1983; “High Performance Pigments,” 2002; “Industrial Inorganic Pigments,” 1998; “Industrial Organic Pigments, Second, Completely Revised Edition,” 1993).

Specific standards for physical properties, chemical properties, purity, and/or procedures for testing the purity/properties of various pigments (e.g., lead chromate, chromium oxide, phthalocyanine green, a phthalocyanine blue, molybdate orange, white zinc, zinc oxide, calcium carbonate, barium sulfate, aluminum silicate, diatomaceous silica, magnesium silicate, mica, calcium borosilicate, zinc hydroxy phosphite, aluminum powder, micaceous iron oxide, zinc phosphate, basic lead silicochromate, strontium chromate, ochre, lampblack, orange shellac, raw umber, burnt umber, raw sienna, burnt sienna, bone black, carbon black, red iron oxide, brown iron oxide, basic carbonate, white lead, white titanium dioxide, iron blue, ultramarine blue, chrome yellow, chrome orange, hydrated yellow iron oxide, zinc chromate yellow, red lead, para red toner, toluidine red toner, chrome oxide green, zinc dust, cuprous oxide, mercuric oxide, iron oxide, anhydrous aluminum silicate, black synthetic iron oxide, gold bronze powder, aluminum powder, strontium chromate pigment, basic lead silicochromate) for use in a coating are described, for example in “ASTM Book of Standards, Volume 06.03, Paint—Pigments, Drying Oils, Polymers, Resins, Naval Stores, Cellulosic Esters, and Ink Vehicles,” D280-01, D2448-85, D126-87, D305-84, D3021-01, D3256-86, D2218-67, D3280-85, D50-90, D79-86, D1199-86, D602-81, D715-86, D603-66, D718-86, D604-81, D719-91, D605-82, D717-86, D607-82, D716-86, D4288-02, D4487-90, D4462-02, D4450-85, D962-81, D5532-94, D6280-98, D1648-86, D1649-01, D85-87, D209-81, D237-57, D763-01, D765-87, D210-81, D561-82, D3722-82, D3724-01, D34-91, D81-87, D1301-91, D1394-76, D261-75, D262-81, D1135-86, D211-67, D768-01, D444-88, D3872-86, D478-02, D1208-96, D83-84, D49-83, D3926-80, D475-67, D656-87, D970-86, D3721-83, D263-75, D520-00, D521-02, D283-84, D284-88, D3720-90, D3619-77, D769-01, D476-00, D267-82, D480-88, D1845-86, D1844-86, and D279-O₂, 2002; and in “ASTM Book of Standards, Volume 06.01, Paint—Tests for Chemical, Physical, and Optical Properties; Appearance,” D5381-93 and D6131-97 2002.

(1) Corrosion Resistance Pigments

Addition of certain pigments may improve the corrosion resistance of a coating and/or film, or specifically, the protection of a metal surface coated with a coating and/or film from corrosion. Often, a primer comprises such pigments. Examples of corrosion resistance pigments include aluminum flake, aluminum triphosphate, aluminum zinc phosphate, ammonium chromate, barium borosilicate, barium chromate, barium metaborate, basic calcium zinc molybdate, basic carbonate white lead, basic lead silicate, basic lead silicochromate, basic lead silicosulfate, basic zinc molybdate, basic zinc molybdate-phosphate, basic zinc molybdenum phosphate, basic zinc phosphate hydrate, bronze flake, calcium barium phosphosilicate, calcium borosilicate, calcium chromate, calcium plumbate (CI Pigment Brown 10), calcium strontium phosphosilicate, calcium strontium zinc phosphosilicate, dibasic lead phosphite, lead chromosilicate, lead cyanamide, lead suboxide, lead sulfate, mica, micaceous iron oxide, red lead (CI Pigment Red 105), steel flake, strontium borosilicate, strontium chromate (CI Pigment Yellow 32), tribasic lead phophosilicate, zinc borate, zinc borosilicate, zinc chromate (CI Pigment Yellow 36), zinc dust (CI Pigment Metal 6), zinc hydroxy phosphite, zinc molybdate, zinc oxide, zinc phosphate (CI Pigment White 32), zinc potassium chromate, zinc silicophosphate hydrate, zinc tetraoxylchromate, or a combination thereof.

The selection of a corrosion resistant pigment may be made based on the mechanism of corrosion resistance it confers to a coating and/or film. Corrosion often occurs as a cathodic process wherein a metal surface acts as a cathode and passes electrons to an electron accepter moiety of a corrosive chemical, such as, for example, hydrogen, oxygen, or a combination thereof. Corrosion can also occur as an anodic process wherein ionized metal atoms then enter solution. Pigments such as, for example, mica, micaceous iron oxide, metallic flake pigments (e.g., aluminum, bronze, steel), or a combination thereof confer corrosion resistance to a coating and/or film by acting as a physical barrier between a metal surface and corrosive chemicals. However, a chemically reactive pigment such as a metal flake pigment be used in an environment at or near neutral pH (e.g., pH 6 to pH 8). Micaceous iron oxide can be selected for a primer, a topcoat, or a combination thereof, and can also function as a UV absorber. Aluminum flake may be selected for an industrial coating, an automotive coating, an architectural coating, a primer, or a combination thereof. Aluminum flake may additionally confer heat resistance, moisture resistance, UV resistance, or a combination thereof to a coating and/or film. Aluminum flake may also be stearate modified for use in a topcoat. However, aluminum flake may produce gas in a coating comprising more than 0.15% water. A metallic zinc pigment (e.g., zinc flake, zinc dust) acts by functioning as an anode instead of the metal surface (e.g., steel). However, the effectiveness of a coating's corrosion resistance fades as the zinc pigment is used up in protective reactions. A metallic zinc primer may be selected for a primer, particularly in combination with an epoxy topcoat, a urethane topcoat, or a combination thereof.

Red lead and/or basic lead silicochromate can confer an orange color, and may be selected for combination with an oil-based coating (e.g., a primer), as the pigment chemically reacts with an oil-based binder to produce a corrosion resistant lead soap in the coating and/or film. Red lead and/or basic lead is typically selected for a primer in an industrial steel coating.

A barium metaborate pigment acts by retarding an anodic process. A barium metaborate pigment is usual chemically modified by combination with silica to reduce solubility. A zinc borate combined with a zinc phosphate, a modified barium metaborate, or combination thereof demonstrates synergistic enhancement of corrosion resistance, as well as flame retardancy.

Zinc potassium chromate may confer a yellow color as well as an anticorrosive property. Zinc tetraoxylchromate can also confer a yellow color, and is typically selected for use in a two pack poly(viny butyryl) primer. Zinc oxide may be selected for an oleoresinous coating, a water-borne coating, a primer, or a combination thereof, and may be combined with a zinc chromate and/or calcium borosilicate, and additionally may improve thermosetting crosslinking density and/or act as a UV absorber. Strontium chromate may confer a yellow color, and may be selected for an aluminum surface, an aircraft primer, or a combination thereof. Strontium chromate may be combined with a zinc chromate in a water-borne coating, though in some embodiments the total chromate content is less from 0.001% to 2%. Ammonium chromate, barium chromate and calcium chromate may be selected as a corrosion inhibitor, particularly as a flash rust inhibitor.

A zinc molybdate, zinc phosphate, zinc hydroxy phosphite, or a combination thereof may confer a white color. These zinc pigments function by reducing an anodic process, though zinc hydroxy phosphite may form corrosion resistant soap in an oleoresinous-coating. Basic zinc molybdate typically is selected for an alkyd-coating, an epoxide-coating, an epoxy ester-coating, a polyester-coating, a solvent-borne coating, or a combination thereof. Basic zinc molybdate-phosphate is similar to basic zinc molybdate, though it may provide superior corrosion resistance for a rusted steel surface. Basic calcium zinc molybdate may be selected for a water-borne coating, a two-pack polyurethane coating, a two-pack epoxy coating, or a combination thereof. A combination of basic calcium zinc molybdate and zinc phosphate may confer a superior adhesion property to a surface comprising iron, and may be selected for a water-borne coating or a solvent-borne coating. A zinc phosphate may be selected for an alkyd coating, a water-reducible coating, a coating cured by an acid and baking, or a combination thereof. A zinc phosphate is often less selected for a marine coating for salt water embodiments. A modified zinc phosphate, such as, for example, aluminum zinc phosphate, basic zinc phosphate hydrate, zinc silicophosphate hydrate, basic zinc molybdenum phosphate, or a combination thereof may confer improved corrosion resistance for a salt water embodiment. Zinc hydroxy phosphite may be selected for a solvent-borne coating.

An aluminum triphosphate typically confers a white color, acts by chelating iron ions, and may be used for a surface that comprises iron. A grade I aluminum triphosphate is modified with zinc and silicate, and may be selected for an alkyd-coating, an epoxy coating, a solvent-borne coating, a primer, or a combination thereof. A grade II aluminum triphosphate is modified with zinc and silicate, and may be selected for a water-borne coating or a solvent-borne coating. A grade III aluminum triphosphate is modified with zinc, and may be selected for a water-borne coating or a solvent-borne coating.

A silicate pigment such as barium borosilicate, calcium borosilicate, strontium borosilicate, zinc borosilicate, a calcium barium phosphosilicate, a calcium strontium phosphosilicate, a calcium strontium zinc phosphosilicate, or a combination thereof, typically acts through inhibiting an anodic or cathodic process, as well as forming a corrosion resistant soap in an oleoresinous-coating. A grade I and/or III calcium borosilicate may be selected for a medium oil alkyd-coating, a long oil alkyd, an epoxy ester-coating, a solvent-borne coating, an architectural coating, an industrial coating, or a combination thereof, but may be less selected for a marine coating, an epoxide-coating, a water-borne coating, or a combination thereof. Calcium barium phosphosilicate grade I pigment may be selected for a solvent-borne epoxy-coating, to confer an antisettling property to a primer comprising zinc, or a combination thereof. Calcium barium phosphosilicate grade II pigment may be selected for a water-borne coating, an alkyd-coating, or a combination thereof. Calcium strontium phosphosilicate may be selected for a water-borne acrylic lacquer, a water-borne sealant, or a combination thereof. In aspects wherein a water-borne acrylic lacquer comprises calcium strontium phosphosilicate, it is contemplated that a 1:1 ratio of zinc phosphate pigment is included. Calcium strontium zinc phosphosilicate may be selected for an alkyd-coating, an epoxide coating, a coating cured by a catalyst and baking, a water-borne coating, or a combination thereof.

(2) Camouflage Pigments

A camouflage pigment refers to a pigment typically selected to camouflage a surface (e.g., a military surface) from visual and, in specific facets, infrared detection. Examples of a camouflage pigment include an anthraquinone black, a chromium oxide green, or a combination thereof. A chromium oxide green may be selected for embodiments wherein good chemical resistance, dull color, good heat stability, good infrared reflectance, good light fastness, good opacity, good solvent resistance, low tinctorial strength, or a combination thereof, is suitable. Anthraquinone black (CI Pigment Black 20) may be selected for good light fastness and moderate solvent resistance, and is often selected for camouflage coatings, due to its infrared absorption property.

(3) Color Property Pigments

A color property is the ability of a composition to confer a visual color and/or metallic appearance to a coating and/or a coated surface. Color pigments are often categorized by a common name recognized within the art, which often encompasses several specific color pigments, each identified by a CI number.

(i) Black Pigments

A black pigment is a pigment that confers a black color to a coating. Examples of black pigments, identified by common name with examples of specific pigments in parentheses, include aniline black; anthraquinone black; carbon black; copper carbonate; graphite; iron oxide; micaceous iron oxide; manganese dioxide; or a combination thereof.

Aniline black (e.g., CI Pigment Black 1); may be selected for a deep black color (e.g., strong light absorption, low light scattering) and/or fastness. Coatings comprising aniline black typically comprise relatively higher concentrations of binder, and thus often possesses a matt property.

Anthraquinone black (e.g., CI Pigment Black 20) may be selected for good light fastness and moderate solvent resistance.

Carbon black (e.g., CI Pigment Black 6, CI Pigment Black 7, CI Pigment Black 8) generally possesses properties such as chemical stability, good light fastness, good solvent resistance, heat stability, or a combination thereof. Carbon black is often categorized into separate grades, based on the intensity of black color (“jetness”). To reduce flocculation in preparing a coating comprising a carbon black pigment, such pigments may be incrementally added to a coating during preparation, chemically modified by surface oxidation, chemically modified by an organic compound (e.g., a carboxylic acid), or a combination thereof. Additionally, a carbon black pigment may absorb certain other coating components such as a metal soap drier. Typically, increasing the concentration of the susceptible component by, for example, two-fold will reduce this effect. A high jet channel black pigment is often selected for use in an automotive coating wherein a high jetness is desired. The other grades of carbon black pigments are often selected for architectural coatings.

Graphite (e.g., CI Pigment Black 10) may be selected for properties such as relative chemically inertness, low in color intensity, low in tinctorial strength, an anti-corrosive property, an increase in coating spreading rate, or a combination thereof.

Iron oxide (e.g., CI Pigment Black 11) may be selected for properties such as good chemical resistance, relative inertness, good solvent resistance, limited heat resistance, low tinctorial strength, or a combination thereof. Iron oxide possesses superior floating resistance than carbon black, particularly in combination with titanium dioxide.

Micaceous iron oxide may be selected for properties such as relative inertness, grayish appearance, shiny appearance, function as a UV absorber, function as an anti-corrosive pigment due to resistance to oxygen and moisture passage. However, over-dispersal of a micaceous iron oxide during coating preparation may damage the pigment.

(ii) Brown Pigments

A brown pigment is a pigment that confers a brown color to a coating. Examples of a brown pigment include azo condensation (CI Pigment Brown 23, CI Pigment Brown 41, CI Pigment Brown 42); benzimidazolone (CI Pigment Brown 25); iron oxide; metal complex brown; or a combination thereof. A synthetically produced iron oxide brown (CI Pigment Brown 6, CI Pigment Brown 7) may be selected for embodiments wherein a rich brown color, good lightfastness, or a combination thereof is suitable. A metal complex brown (CI Pigment Brown 33) may be selected for embodiments wherein high heat stability, good fastness, or a combination thereof is suitable. A metal complex brown may be used, for example, in a coil coating, a coating for a ceramic surface, or a combination thereof.

(iii) White Pigments

A white pigment is a pigment that confers a white color to a coating. Examples of a white pigment include antimony oxide; basic lead carbonate (CI Pigment White 25); lithopone; titanium dioxide; white lead; zinc oxide; zinc sulphide (CI Pigment White 7); or a combination thereof.

Antimony oxide (CI Pigment White 11) is chemically inert, and used in fire resistant coatings. In some embodiments, antimony oxide may be combined with titanium dioxide, particularly in a coating where chalking is undesirable and a white color in the coating is desired.

Titanium dioxide (CI Pigment White 6) is resistant to heat, many chemicals, and organic solvents, allowing use in many different applications where such properties are desirable. Titanium dioxide may be in the form of a crystal, such as an anatase crystal, a rutile crystal, or a combination thereof. Rutile is more opaque than anatase. Anatase has a greater ability to chalk and is whiter in color than rutile. In aspects wherein chalking is undesirable, a titanium dioxide crystal may be reacted with an inorganic oxide to enhance chalking resistance. Examples of such inorganic oxides include aluminum oxide, silicon oxide, zinc oxide, or a combination thereof.

White lead (CI Pigment White 1) is chemically reactive with acidic binders to form strong films with elastic properties, but also chemically reacts with sulphur to become black in color. It is less selected in certain coatings due to the toxic nature of lead.

Zinc oxide (CI Pigment White 4) confers desirable properties such as resistance to mildew, as well as chemically reacting with oleoresin binders in film formation to enhance resistance to abrasion, to enhance resistance to moisture, to enhance hardness, and/or reduce chalking. However, these reactions may undesirably occur during storage. In some embodiments, it may be combined with titanium dioxide, particularly in a coating comprising an oleoresin binder when chalking is undesirable and a white color in the coating is desired.

Zinc sulfide (CI Pigment White 7) is chemically inert, and confers a strong chalking property. In certain embodiments, a zinc sulfide comprises a lithopone. A lithopone (CI Pigment White 5) comprises a mixture of ZnS and barium sulphate (BaSO₄), usually from 30% to 60% ZnS and 70% to 40% BaSO₄.

(iv) Pearlescent Pigments

A pearlescent pigment is a pigment that confers a pearl-like appearance to a coating. Examples of a white pigment include titanium dioxide and ferric oxide covered mica, bismuth oxychloride crystal, or a combination thereof.

(v) Violet Pigments

A violet pigment is a pigment that confers a violet color to a coating. However, a violet pigment is often used in combination with a red pigment or a blue pigment to produce a desirable color of an intermediate hue between red and blue. Additionally, a violet pigment is often combined with titanium dioxide to balance the slight yellow color of that white pigment. An example of a violet pigment includes dioxanine violet (CI Pigment Violet 23; CI Pigment Violet 37). A dioxazine violet may be selected for embodiments wherein high heat stability, good light fastness, good solvent fastness, or a combination thereof is suitable. CI Pigment Violet 23 (“carbazole violet”) is relatively transparent and bluer than CI Pigment 37, and is typically used in a metallic coating. A dioxazine violet is susceptible to flocculation, loss in a powder coating, or a combination thereof, due to small particle size.

(vi) Blue Pigments

A blue pigment is a pigment that confers a blue color to a coating. Examples of a blue pigment include carbazol Blue; carbazole Blue; cobalt blue; copper phthalocyanine; dioxanine Blue; indanthrone; phthalocyanin blue; Prussian blue; ultramarine; or a combination thereof.

A cobalt blue (CI Pigment Blue 36) may be selected for embodiments wherein good chemical resistance, good lightfastness, good solvent fastness, or a combination thereof, is suitable. An indanthrone (CI Pigment Blue 60) may be selected for embodiments wherein a redish-blue hue, good chemical resistance, good heat resistance, good solvent fastness, transparency, superior resistance to flocculation relative to a copper phthalocyanine, or a combination thereof, is suitable.

A copper phthalocyanine (CI Pigment Blue 15, CI Pigment Blue 15:1, CI Pigment Blue 15:2, CI Pigment Blue 15:3, CI Pigment Blue 15:4, CI Pigment Blue 15:6, CI Pigment Blue 16) may be selected for embodiments wherein good color strength, good tinctorial strength, good heat stability, good lightfastness, good solvent resistance, transparency, or a combination thereof, is suitable. CI Pigment Blue 15 is redish in hue, but is chemically unstable upon contact with an aromatic hydrocarbon, and converts to a greenish blue compound. CI Pigment Blue 15:1 is a form of CI Pigment Blue 15 chemically stabilized by chlorination, greener, and tinctorially weaker than CI Pigment Blue 15. CI Pigment Blue 15:2 is a modified form of CI Pigment Blue 15 that is resistant to flocculation. CI Pigment Blue 15:3 is greenish-blue, while CI Pigment Blue 15:4 is a modified form of CI Pigment Blue 15:3 that is resistant to flocculation. CI Pigment Blue 16 is relatively transparent. Examples of coatings wherein copper phthalocyanine is used include a metallic automotive coating. However, as described above, a copper phthalocyanine may be susceptible to flocculation due to small primary particle size, and various modified forms are known wherein flocculation is reduced. Examples of modifications used to reduce flocculation adding a sulfonic acid moiety; a sulfonic acid moiety and a long chain amine moiety; an aluminum benzoate; an acidic binder (e.g., a rosin); a chloromethyl moiety; or a combination thereof, to the phthalocyanine. A modified phthalocyanine may be selected for embodiments wherein superior color shade, dispersibility, gloss, or a combination thereof is suitable.

A Prussian blue (CI Pigment Blue 27) may be selected for embodiments wherein a strong color, good heat stability, good solvent fastness, or a combination thereof is suitable. However, a Prussian blue is chemically unstable in alkali conditions. An ultramarine (CI Pigment Blue 29) may be selected wherein a strong color, good heat stability, good light fastness, good solvent resistance, or a combination thereof is suitable. However, an ultramarine is chemically unstable in acidic conditions.

(vii) Green Pigments

A green pigment is a pigment that confers a green color to a coating. However, often a “green pigment” comprises a mixture of a yellow pigment and a blue pigment, with the properties of each component pigment generally retained. Examples of a green pigment include chrome green; chromium oxide green; halogenated copper phthalocyanine; hydrated chromium oxide; phthalocyanine green; or a combination thereof.

A chrome green (“Brunswick green,” CI Pigment Green 15) comprises a combination of a Prussian blue and/or a copper phthalocyanine blue and a chrome yellow. A coating comprising a chrome green may be susceptible to floating and flooding defects. A chromium oxide green (CI Pigment Green 17) may be selected for embodiments wherein good chemical resistance, dull color, good heat stability, good infrared reflectance, good light fastness, good opacity, good solvent resistance, low tinctorial strength, or a combination thereof is suitable. A hydrated chromium oxide (CI Pigment Green 18) is similar to chromium oxide, and may be selected for embodiments wherein good light fastness, relatively brighter appearance, relatively greater transparency, relatively less heat stability, relatively less acid stability, or a combination thereof, is suitable. A phthalocyanine green (CI Pigment Green 7, CI Pigment Green 36) may be selected for embodiments wherein good chemical resistance, good heat stability, good light fastness, good solvent resistance, good tinctorial strength, color transparency, or a combination thereof is suitable. CI Pigment Green 7 may be selected for a bluish green color, while CI Pigment Green 36 may be selected for a yellower-greenish color. A phthalocyanine green is often selected for an automotive coating (e.g., a metallic coating), an industrial coating, an architectural coating, a powder coating, or a combination thereof.

(viii) Yellow Pigments

In certain embodiments, a coating may comprise a yellow pigment. A “yellow pigment” is a pigment that confers a yellow color to a coating. Examples of a yellow pigment include anthrapyrimidine; arylamide yellow; barium chromate; benzimidazolone yellow; bismuth vanadate (CI Pigment Yellow 184); cadmium sulfide yellow (CI Pigment Yellow 37); complex inorganic color pigment; diarylide yellow; disazo condensation; flavanthrone; isoindoline; isoindolinone; lead chromate; nickel azo yellow; organic metal complex; quinophthalone; yellow iron oxide; yellow oxide; zinc chromate; or a combination thereof.

An anthrapyrimidine pigment (CI Pigment Yellow 108) may be selected for embodiments wherein, moderate light fastness, moderate solvent resistance, a dull color, transparency, or a combination thereof is suitable.

An arylamide yellow (“Hansa® yellow,” CI Pigment Yellow 1, CI Pigment Yellow 3, CI Pigment Yellow 65, CI Pigment Yellow 73, CI Pigment Yellow 74, CI Pigment Yellow 75, CI Pigment Yellow 97, CI Pigment Yellow 111) may be selected for embodiments wherein, poor heat stability, good light fastness, poor solvent resistance, moderate tinctorial strength, or a combination thereof is suitable. CI Pigment 1 and CI Pigment 74 are mid-yellow in hue. CI Pigment Yellow 3 is greenish in hue. CI Pigment Yellow 73 is mid-yellow in hue, and resistant to recrystallization during dispersion. CI Pigment 97 possesses superior solvent fastness than other arylamide yellow pigments, and has been used in a stoving enamel, an automotive coating, or a combination thereof. Other arylamide yellow pigments may be used in a water-borne coating, a coating comprising a white spirit liquid component, or a combination thereof.

A benzimidiazolone yellow (CI Pigment Yellow 120, CI Pigment Yellow 151, CI Pigment Yellow 154, CI Pigment Yellow 175, CI Pigment Yellow 181, CI Pigment Yellow 194) may be selected for embodiments wherein, good chemical resistance, good heat stability, good light fastness, good solvent resistance, or a combination thereof is suitable. A benzimidiazolone with larger particle size been used in an automotive coating, a powder coating, or a combination thereof.

A cadmium sulfide yellow (CI Pigment Yellow 37) may be selected for embodiments wherein good stability in basic pH, good heat stability, good light fastness, good opacity, good solvent fastness, or a combination thereof is suitable. However, a cadmium yellow comprises cadmium, which may limit suitability relative to an environmental law or regulation.

A complex inorganic color pigment (“mixed phase metal oxide,” CI Pigment Yellow 53, CI Pigment Yellow 119, CI Pigment Yellow 164); may be selected for embodiments wherein, good chemical stability, good heat resistance, good light fastness, good opacity, good solvent fastness, or a combination thereof is suitable. However, a complex inorganic color pigment generally produces a pale color, and is often combined with an additional pigment (e.g., an organic pigment). A complex inorganic color pigment is often selected for an automotive coating, a coil coating, or a combination thereof. A bismuth vanadate is similar to a complex inorganic pigment, but possesses superior color of green-yellow hue, poorer light fastness, and greater use in a powder coating. A bismuth vanadate is often combined with a light stabilizer.

A diarylide yellow (CI Pigment Yellow 12, CI Pigment Yellow 13, CI Pigment Yellow 14, CI Pigment Yellow 17, CI Pigment Yellow 81, CI Pigment Yellow 83) may be selected for embodiments wherein, good chemical resistance, poor light fastness, good solvent resistance, good tinctorial strength, or a combination thereof is suitable. A diarylide yellow is not stable at a temperature of 200° C. or greater. CI Pigment Yellow 83 has superior light fastness than other diarylide yellow pigments, and has been used in an industrial coating, a powder coating, or a combination thereof.

A diazo condensation pigment (CI Pigment Yellow 93, CI Pigment Yellow 94, CI Pigment Yellow 95, CI Pigment Yellow 128, CI Pigment Yellow 166) may be selected for embodiments wherein, good chemical resistance, good heat stability, good solvent resistance, good tinctorial strength, or a combination thereof is suitable. A diazo condensation pigment typically is used in plastics, though CI Pigment Yellow 128 has been used in a coating such as an automotive coating.

A flavanthrone pigment (CI Pigment Yellow 24) may be selected for embodiments wherein, good heat stability, moderate light fastness, a reddish yellow hue superior to an anthrapyrimidine, transparency, or a combination thereof is suitable.

An isoindoline yellow pigment (CI Pigment Yellow 139, CI Pigment Yellow 185) may be selected for embodiments wherein, good chemical resistance, good heat stability, good light fastness, good solvent resistance, moderate tinctorial strength, or a combination thereof is suitable. An isoindolinone yellow pigment (CI Pigment Yellow 109, CI Pigment Yellow 110, CI Pigment Yellow 173) typically has been used in an automotive coating or an architectural coating. An isoindoline yellow pigment may be selected for embodiments wherein, good light fastness, good tinctorial strength, or a combination thereof is suitable. However, an isoindoline pigment is not stable in a basic pH. An isoindoline yellow pigment typically has been used in an industrial coating.

A lead chromate (CI Pigment Yellow 34) may be selected for embodiments wherein moderate heat stability, low oil absorption, good opacity, good solvent resistance, or a combination thereof is suitable. However, a lead chromate is susceptible to an acidic or a basic pH, and a lower light fastness so that the pigment darkens upon irradiation by light. The pH and lightfastness properties of commercially produced lead chromate are often improved by treatment of a lead chromate with silica, antimony, alumina, metal, or a combination thereof. Additionally, a lead chromate comprises lead and/or chromium, which may limit suitability relative to an environmental law or regulation. A lead chromate may comprise a lead sulfate, which is used to modify color. Examples of lead chromates include a lemon chrome, which comprises from 20% to 40% lead sulfate and is greenish yellow in color; a middle chrome, which comprises little lead sulfate and is reddish yellow in color; orange chrome, which comprises no detectable lead sulfate; and primrose chrome, which comprises from 45% to 55% lead chrome and is greenish yellow in color.

An organic metal complex (CI Pigment Yellow 129, CI Pigment Yellow 153) may be selected for embodiments wherein good solvent resistance is suitable. An organic metal complex typically is transparent and dull in color.

A quinophthalone pigment (CI Pigment Yellow 138) may be selected for embodiments wherein, good heat stability, good light fastness, good solvent resistance, a reddish yellow hue, or a combination thereof is suitable. A quinophthalone can be either opaque or transparent. A quinophthalone pigment has been used as a substitute for chrome as a pigment.

A yellow iron oxide (CI Pigment Yellow 42, CI Pigment Yellow 43) may be selected for embodiments wherein good covering power, good disperability, good resistance to chemicals, good light fastness, good solvent resistance, a yellow with greenish hue is desired, or a combination thereof is suitable. A yellow iron oxide can function as a U.V. absorber. However, a yellow iron oxide is generally of duller color relative to other pigments, and is susceptible to temperatures of 105° C. or greater. Additionally, a yellow iron oxide may comprise a α-crystal, a β-crystal, a γ-crystal, or a combination thereof. Overdispersion may damage the needle-shape crystal structure, which can reduce the color intensity. Additionally, a transparent yellow iron oxide can be prepared by selecting particles with minimum size, and such a pigment is used, for example, in an automotive coating or a wood coating.

(ix) Orange Pigments

In certain embodiments, a coating may comprise an orange pigment. An “orange pigment” is a pigment that confers an orange color to a coating. Examples of an orange pigment include perinone orange; pyrazolone orange; or a combination thereof.

A perinone orange pigment (CI Pigment Orange 43) may be selected for embodiments wherein very good resistance to heat, good light fastness, good solvent resistance, high tinctorial strength, or a combination thereof is suitable.

A pyrazolone orange pigment (CI Pigment Orange 13, CI Pigment Orange 34) is similar to a diarylide yellow pigment, and may be selected for embodiments wherein moderate resistance to heat, poor light fastness, moderate solvent resistance, high tinctorial strength, or a combination thereof is suitable. However, CI Pigment Orange 34 possesses greater lightfastness relative to CI Pigment Orange 13, and has been used in an industrial coating and/or a replacement for chrome.

(x) Red Pigments

In certain embodiments, a coating may comprise a red pigment. A “red pigment” is a pigment that confers a red color to a coating. Examples of an red pigment include anthraquinone; benzimidazolone; BON arylamide; cadmium red; cadmium selenide; chrome red; dibromanthrone; diketopyrrolo-pyrrole pigment (CI Pigment Red 254, CI Pigment Red 255, CI Pigment Red 264, CI Pigment Red 270, CI Pigment Red 272); disazo condensation pigment (CI Pigment Red 144, CI Pigment Red 166, CI Pigment Red 214, CI Pigment Red 220, CI Pigment Red 221, CI Pigment Red 242); lead molybdate; perylene; pyranthrone; quinacridone; quinophthalone; red iron oxide; red lead; toluidine red; tonor pigment (CI Pigment Red 48, CI Pigment Red 57, CI Pigment Red 60, CI Pigment Red 68); β-naphthol red; or a combination thereof.

A lead molybdate red pigment (CI Pigment Red 104) may be selected for embodiments wherein good resistance to heat, moderate resistance to basic pH, good opacity, excellent solvent resistance, or a combination thereof is suitable. A molybdate red is bright in color, and is often combined with an organic pigment to extend a color range. However, a molybdate is easy to disperse, and overdispersion may damage this pigment. Additionally, a molybdate red comprising lead and/or chromium may have limited suitability relative to an environmental law or regulation.

A cadmium red pigment (CI Pigment Red 108) may be selected for embodiments wherein excellent resistance to heat, good lightfastness, poor resistance to acidic pH, good opacity, excellent solvent resistance, or a combination thereof is suitable. However, a cadmium red comprises cadmium, and may have limited suitability relative to an environmental law or regulation.

A red iron oxide pigment (CI Pigment Red 101, CI Pigment Red 102) may be selected for embodiments wherein excellent resistance to heat, good lightfastness, poor resistance to acidic pH, good opacity, excellent solvent resistance, or a combination thereof is suitable. However, a cadmium red comprises cadmium, and may have limited suitability relative to an environmental law or regulation.

β-naphthol red (CI Pigment Red 3) may be selected for embodiments wherein modest heat resistance, good lightfastness, modest solvent resistance, or a combination thereof is suitable.

BON arylamide (CI Pigment Red 2, CI Pigment Red 5, CI Pigment Red 12, CI Pigment Red 23, CI Pigment Red 112, CI Pigment Red 146, CI Pigment Red 170) comprises various pigments that generally have good lightfastness, good solvent resistance, or a combination thereof.

Tonor pigment (CI Pigment Red 48, CI Pigment Red 57, CI Pigment Red 60, CI Pigment Red 68) comprises various pigments that generally have good solvent resistance, but often have poor acid resistance, poor alkali resistance, or a combination thereof.

Benzimidazolone (CI Pigment Red 171, CI Pigment Red 175, CI Pigment Red 176, CI Pigment Red 185, CI Pigment Red 208) comprises various pigments that generally have good heat stability, excellent solvent resistance, or a combination thereof.

Disazo condensation pigment (CI Pigment Red 144, CI Pigment Red 166, CI Pigment Red 214, CI Pigment Red 220, CI Pigment Red 221, CI Pigment Red 242) comprises various pigments that generally have excellent heat stability, good solvent resistance, or a combination thereof.

Quinacridone (CI Pigment Red 122, CI Pigment Red 192, CI Pigment Red 202, CI Pigment Red 207, CI Pigment Red 209) comprises a various pigments that generally have bright color, excellent heat stability, excellent solvent resistance, excellent chemical resistance, good lightfastness, or a combination thereof.

Perylene (CI Pigment Red 123, CI Pigment Red 149, CI Pigment Red 178, CI Pigment Red 179, CI Pigment Red 190, CI Pigment Red 224) comprises a various pigments that generally have excellent heat stability, excellent solvent resistance, excellent lightfastness, or a combination thereof.

Anthraquinone (CI Pigment Red 177) has a bright color, good heat stability, good solvent resistance, good lightfastness, or a combination thereof.

Dibromanthrone (CI Pigment Red 168) has a bright color, moderate heat stability, good solvent resistance, excellent lightfastness, or a combination thereof.

Pyranthrone (CI Pigment Red 216, CI Pigment Red 226) has a dull color, moderate heat stability, good solvent resistance, poor lightfastness in combination with titanium dioxide, or a combination thereof.

Diketopyrrolo-pyrrole pigment (CI Pigment Red 254, CI Pigment Red 255, CI Pigment Red 264, CI Pigment Red 270, CI Pigment Red 272) comprises a various pigments that generally have a bright color, good opacity, excellent heat stability, excellent solvent resistance, or a combination thereof

(xi) Metallic Pigments

In certain embodiments, a coating may comprise a metallic pigment. A “metallic pigment” is a pigment that confers a metallic appearance to a coating, and as previously described, is often a corrosion resistance pigment. A metallic pigment may be selected for a topcoat, particularly to confer a metallic appearance, a primer, particularly to confer a corrosion resistance property, an automotive coating, an industrial coating, or a combination thereof. Metallic flake pigments may be selectd for embodiments wherein UV and/or infrared resistance is to be conferred to a coating. Additionally, as some enzymes comprise a metal atom in the active site, inclusion of a metallic pigment and/or other composition comprising a metal during coating preparation, or addition later (e.g., a multipack coating) may stimulate a desired enzyme activity. Examples of a metallic pigment include aluminum flake (CI Pigment Metal 1); aluminum non-leafing, gold bronze flake, zinc dust, stainless steel flake, nickel (e.g., flake, powder), or a combination thereof.

(4) Extender Pigments

An extender pigment (“inert pigment,” “extender,” “inert,” “filler”) is a substance that is insoluble in the other components of a coating, and further confers a desirable optical property (e.g., opacity, gloss), a rheological property, physical property, an antisettling property, or a combination thereof, to the coating and/or film. An extender pigment is often white or near white in color, and typically are used to provide a cheap partial substitute for a more expensive white pigment (e.g., titanium dioxide). Often an extender has a refractive index below 1.7. In some aspects, an extenders refractive index is 1.30 to 1.70, including all intermediate ranges and combinations thereof. Examples of an inorganic extender include a barium sulphate (CI Pigment White 21, CI Pigment White 22); 1); a calcium carbonate (CI Pigment White 18); a calcium sulphate; a silicate (CI Pigment White 19, CI Pigment White 26); a silica (CI Pigment White 27); or a combination thereof.

Calcium carbonate (“calcite,” “whiting,” “limestone,” CI Pigment White 18) is generally chemically inert with the exception of reactions with an acid. Calcium carbonate may be used in a water-borne coating or a solvent-borne coating. Properties specifically associated with calcium carbonate include conferring settling resistance, sag resistance, or a combination thereof. Precipitated calcium carbonate obtained from processing of limestone, and may have superior opacity.

Kaolin (“china clay”) is typically selected for a latex coating, an alkyd coating, an architectural coating, or a combination thereof. In addition to the typical properties of an extender (e.g., opacity), kaolin can confer scrub resistance to a coating.

Talc is a hydrated magnesium aluminum silicate, and is soluble in water. Talc may be selected for an architectural coating (e.g., interior, exterior), a primer, a traffic marker coating, an industrial coating, or a combination thereof. Talc comprising a platy particle shape can confer chemical resistance, water resistance, improved flow property, or a combination thereof.

Silica is silicon dioxide, and may be classified as crystalline silica, diatomaceous silica or synthetic silica. Crystalline silica is produced from crushed and ground quartz, and may be selected for an architectural coating, an industrial coating, a primer, a latex coating, a powder coating, or a combination thereof. Crystalline silica may confer burnish resistance to a coating and/or film. Diatomaceous silica (“diatomaceous earth,” “diatomite”) is the mineral fossil of diatoms which were single celled aquatic plants. Diatomaceous silica may be selected for an architectural coating, a latex coating, or a combination thereof. Diatomaceous silica may also function as a flattening agent. Synthetic silica is produced from chemical reactions, and includes, for example, precipitated silica, fumed silica, or a combination thereof. Precipitated silica may be selected for an industrial coating, a solvent-borne coating, or a combination thereof. Precipitated silica may also function as a flattening agent. Fumed silica may be selected for an industrial coating. Fumed silica may also function as a flattening agent, a rheology modifier, or a combination thereof.

Mica is a hydrous silica aluminum potassium silicate, and typically comprises plate shaped particles. Mica may be selected for an architectural coating, an exterior coating, a traffic marker coating, a primer, or a combination thereof. Mica may also confer durability, moisture resistance, corrosion resistance, heat resistance, chemical resistance, cracking resistance, sagging resistance, or a combination thereof, to a coating and/or film.

Barium sulfate may be classified as baryte or a blanc fixe. Baryte may be selected for an automotive coating, an industrial coating, a primer, an undercoat, or a combination thereof. Blanc fixe has good opacity for an extender, and may be selected for an automotive coating, an industrial coating, or a combination thereof.

Wollastonite is a calcium metasilicate, and may be selected for a latex coating. Wollasonite may also function as an alkali pH buffer. Surface modified wollasonite may be selected for an industrial coating.

Nepheline syenite is an anhydrous sodium potassium aluminum silicate, and may be selected for an architectural coating, a latex coating, an interior coating, an exterior coating, or a combination thereof. Nepheline syenite may function may confer cracking resistance, scrub resistance, or a combination thereof.

Sodium aluminosilicate may be selected for a latex coating, an architectural coating, or a combination thereof. Sodium aluminosilicate may also function as a flattening agent.

Alumina trihydrate may be selected for an architectural coating, a thermoplastic coating, a thermosetting coating, or a combination thereof. Alumina trihydrate may confer flame retardancy to a film.

b. Dyes

A dye is a composition that is soluble in the other components of a coating, and further confers a desirable color property to the coating. It is contemplated that many of the compounds that give a biomolecular composition (e.g., a microorganism derived particulate material) color, such as photosynthetic pigment and/or carotenoid pigment, will be partly or fully soluble in many non-aqueous liquids described herein. It is further contemplated that a cell-based material is added to a coating comprising such a liquid component, the material may act as a dye, as well as a pigment and/or extender, due to the dissolving of colored compounds into the liquid component.

4. Coating Additives

A coating additive is any material which is added to a coating to confer a desirable property other than that described for a binder, a liquid component, a colorizing agent, or a combination thereof. It is contemplated that, in addition to the examples of additives described herein, any additive in the art, in light of the present disclosures, may be included in a composition.

Examples of coating additives include a biomolecular composition, as well as an antifloating agent, an antiflooding agent, an antifoaming agent, an antisettling agent, an antiskinning agent, a catalyst, a corrosion inhibitor, a film-formation promoter, a leveling agent, a matting agent, a neutralizing agent, a preservative, a thickening agent, a wetting agent, or a combination thereof. The content for an individual coating additive in a coating generally is 0.000001% to 20.0%, including all intermediate ranges and combinations thereof. However, in many embodiments, it is contemplated the concentration of a single additive in a coating will comprise between 0.000001% and 10.0%, including all intermediate ranges and combinations thereof.

a. Preservatives

A coating may comprise a preservative to reduce or prevent the deterioration of a coating and/or film by a microorganism. A microorganism is generally considered a contaminant capable damaging a film and/or coating to the point of suitable usefulness in a given embodiment. An aspect is the suitability of a cell based particulate material, particularly a microorganism based particulate material, for use as a purposefully added coating component. However, it is contemplated that a coating comprising a cell-based particulate material also comprises a preservative. It is contemplated that continued growth of a microorganism from a biomolecular composition would be detrimental to a coating and/or film, and a preservative may reduce or prevent such growth. It is further contemplated that a contaminating microorganism could use the cell-based particulate material as a readily available source of nutrients for growth, and a preservative may reduce or prevent such growth. It is also contemplated that the amount of preservative added to a coating comprising a cell-based particulate material may be increased relative to a preservative content of a similar coating lacking such an added cell-based particulate material. In certain aspects, it is contemplated that the amount of preservative may be increased 1.01 to 10-fold or more, including all intermediate ranges and combinations thereof, the amount of an example of a preservative content described herein or used in the art, in light of the present disclosures.

Examples of preservatives include a biocide, which kills an organism, a biostatic, which reduces or prevents the growth of an organism, or a combination thereof. Examples of a biocide include, for example, a bactericide, a fungicide, an algaecide, or a combination thereof. Examples of bacteria commonly found to contaminate a coating and/or film include Pseudomonas spp., Aerobacter spp., Enterobacter spp., Flavobacterium spp. (e.g., Flavobacterium marinum), Bacillus spp., or a combination thereof. Examples of fungi commonly found to contaminate a coating and/or film include Aureobasidium pullulans, Alternaria dianthicola, Phoma pigmentivora, or a combination thereof. Examples of algae commonly found to contaminate a coating and/or film include Oscillotoria sp., Scytonema sp., Protoccoccus sp., or a combination thereof. Techniques for determining microbial contamination of a coating and/or coating component have been described (see, for example, “ASTM Book of Standards, Volume 06.01, Paint—Tests for Chemical, Physical, and Optical Properties; Appearance,” D5588-97, 2002).

In addition to the disclosures herein, a preservative and use of a preservative in a coating is known to those of skill in the art, and all such materials and techniques for using a preservative in a coating may be used (see, for example, Flick, E. W. “Handbook of Paint Raw Materials, Second Edition,” 263-285 and 879-998, 1989; in “Paint and Coating Testing Manual, Fourteenth Edition of the Gardner-Sward Handbook,” (Koleske, J. V. Ed.), pp 261-267 and 654-661, 1995; in “Paint and Surface Coatings, Theory and Practice, Second Edition,” (Lambourne, R. and Strivens, T. A., Eds.), pp. 193-194, 371-382 and 543-547, 1999; Wicks, Jr., Z. W., Jones, F. N., Pappas, S. P. “Organic Coatings, Science and Technology, Volume 1: Film Formation, Components, and Appearance,” pp. 318-320, 1992; Wicks, Jr., Z. W., Jones, F. N., Pappas, S. P. “Organic Coatings, Science and Technology, Volume 2: Applications, Properties and Performance,” pp. 145, 309, 319-323 and 340-341, 1992; and in “Paints, Coatings and Solvents, Second, Completely Revised Edition,” (Stoye, D. and Freitag, W., Eds.) pp 6, 127 and 165, 1998; and in “Handbook of Coatings Additives,” pp. 177-224, 1987).

A coating, film, surface, or a combination thereof may be detrimentally affected by the presence of a living microorganism. For example, a living microorganism can alter viscosity due to damage to a cellulosic viscosifier; alter a rheological property by increasing the gelling of a coating; produce an undesirable color alteration (“discoloration”) by production of a colorizing agent; produce undesirable gas and increase foam; produce an undesirable odor; lower pH; damage a preservative; produce slime; reduce adhesion by a film; increase corrosion of a metal surface by moisture production by an organism; increase corrosion of a metal surface by film damage; damage a wooden surface by colonization (e.g., fungal colonization); or a combination thereof. These changes can lead to the coating and/or film becoming unsuitable for use. The undesirable growth of a microorganism is generally more prevalent in a water-borne coating, as the solvent component of a solvent borne-coating usually acts as a preservative. However, a film is generally susceptible to such damage by growth of a microorganism after loss of a solvent (e.g., evaporation) during film formation. Additionally, various bacteria (e.g., Bacillus spp.) and fungi produce spores, which are cells that are relatively durable to unfavorable conditions (e.g., cold, heat, dehydration, a biocide), and may persist in a coating and/or film for months or years prior to germinating into a damaging colony of cells.

In certain embodiments, a preservative may comprise an in-can preservative, an in-film preservative, or a combination thereof. An in-can preservative is a composition that reduces or prevents the growth of a microorganism prior to film formation. Addition of an in-can preservative during a water-borne coating production typically occurs with the introduction of water to a coating composition. Typically, an in-can preservative is added to a coating composition for function during coating preparation, storage, or a combination thereof. An in-film preservative is a composition that reduces or prevents the growth of a microorganism after film formation. In many embodiments, an in-film preservative is the same chemical as an in-can preservative, but added to a coating composition at a higher (e.g., two-fold) concentration for continuing activity after film formation.

Examples of preservatives that have been used in coatings include a metal compound (e.g., an organo-metal compound) biocide, an organic biocide, or a combination thereof. Examples of a metal compound biocide include barium metaborate (CAS No. 13701-59-2), which is a fungicide and bactericide; copper(II) 8-quinolinolate (CAS No. 10380-28-6), which is a fungicide; phenylmercuric acetate (CAS No. 62-38-4), tributyltin oxide (CAS No. 56-35-9), which may be less selected for use against Gram-negative bacteria; tributyltin benzoate (CAS No. 4342-36-3), which is a fungicide and bactericide; tributyltin salicylate (CAS No. 4342-30-7), which is a fungicide; zinc pyrithione (“zinc 2-pyridinethiol-N-oxide”; CAS No. 13463-41-7), which is a fungicide; zinc oxide (CAS No. 1314-13-2), which is a fungistatic/fungicide and algaecide; a combination of zinc-dimethyldithiocarbamate (CAS No. 137-30-4) and zinc 2-mercaptobenzothiazole (CAS No. 155-04-4), which acts as a fungicide; zinc pyrithione (CAS No. 13463-41-7), which is a fungicide; a metal soap; or a combination thereof. Examples of metals comprised in a metal soap biocide include copper, mercury, tin, zinc, or a combination thereof. Examples of an organic acid comprised in a metal soap biocide include a butyl oxide, a laurate, a naphthenate, an octoate, a phenyl acetate, a phenyl oleate, or a combination thereof.

An example of an organic biocide that acts as an algaecide includes 2-methylthio-4-tert-butylamino-6-cyclopropylamino-s-triazine (CAS No. 28159-98-0). Examples of an organic biocide that acts as a bactericide include a combination of 4,4-dimethyl-oxazolidine (CAS No. 51200-87-4) and 3,4,4-trimethyloxazolidine (CAS No. 75673-43-7); 5-hydroxy-methyl-1-aza-3,7-dioxabicylco(3.3.0.) octane (CAS No. 59720-42-2); 2(hydroxymethyl)-aminoethanol (CAS No. 34375-28-5); 2-(hydroxymethyl)-amino-2-methyl-1-propanol (CAS No. 52299-20-4); hexahydro-1,3,5-triethyl-s-triazine (CAS No. 108-74-7); 1-(3-chloroallyl)-3,5,7-triaza-1-azonia-adamantane chloride (CAS No. 51229-78-8); 1-methyl-3,5,7-triaza-1-azonia-adamantane chloride (CAS No. 76902-90-4); p-chloro-m-cresol (CAS No. 59-50-7); an alkylamine hydrochloride; 6-acetoxy-2,4-dimethyl-1,3-dioxane (CAS No. 828-00-2); 5-chloro-2-methyl-4-isothiazolin-3-one (CAS No. 26172-55-4); 2-methyl-4-isothiazolin-3-one (CAS No. 2682-20-4); 1,3-bis(hydroxymethyl)-5,5-dimethylhydantoin (CAS No. 6440-58-0); hydroxymethyl-5,5-dimethylhydantoin (CAS No. 27636-82-4); or a combination thereof. Examples of an organic biocide that acts as a fungicide include a parabens; 2-(4-thiazolyl)benzimidazole (CAS No. 148-79-8); N-trichloromethyl-thio-4-cyclohexene-1,2-dicarboximide (CAS No. 133-06-2); 2-n-octyl-4-isothiazoline-3-one (CAS No. 26530-20-1); 2,4,5,6-tetrachloro-isophthalonitrile (CAS No. 1897-45-6); 3-iodo-2-propynyl butyl carbamate (CAS No. 55406-53-6); N-(trichloromethyl-thio)phthalimide (CAS No. 133-07-3); tetrachloroisophthalonitrile (CAS No. 1897-45-6); potassium N-hydroxy-methyl-N-methyl-dithiocarbamate (CAS No. 51026-28-9); sodium 2-pyridinethiol-1-oxide (CAS No. 15922-78-8); or a combination thereof. Examples of a parbens include butyl parahydroxybenzoate (CAS No. 94-26-8); ethyl parahydroxybenzoate (CAS No. 120-47-8); methyl parahydroxybenzoate (CAS No. 99-76-3); propyl parahydroxybenzoate (CAS No. 94-13-3); or a combination thereof. Examples of an organic biocide that acts as an bactericide and fungicide include 2-mercaptobenzo-thiazole (CAS No. 149-30-4); a combination of 5-chloro-2-methyl-3(2H)-isothiazoline (CAS No. 26172-55-4) and 2-methyl-3(2H)-isothiazolone (CAS No. 2682-20-4); a combination of 4-(2-nitrobutyl)-morpholine (CAS No. 2224-44-4) and 4,4′-(2-ethylnitrotrimethylene dimorpholine (CAS No. 1854-23-5); tetra-hydro-3,5-di-methyl-2H-1,3,5-thiadiazine-2-thione (CAS No. 533-74-4); potassium dimethyldithiocarbamate (CAS No. 128-03-0); or a combination thereof. An example of an organic biocide that acts as an algaecide and fungicide includes diiodomethyl-p-tolysulfone (CAS No. 20018-09-1). Examples of an organic biocide that acts as an algaecide, bactericide and fungicide include glutaraldehyde (CAS No. 111-30-8); methylenebis(thiocyanate) (CAS No. 6317-18-6); 1,2-dibromo-2,4-dicyanobutane (CAS No. 35691-65-7); 1,2-benzisothiazoline-3-one (“1,2-benzisothiazolinone”; CAS No. 2634-33-5); 2-(thiocyanomethyl-thio)benzothiazole (CAS No. 21564-17-0); or a combination thereof. An example of an organic biocide that acts as an algaecide, bactericide, fungicide and molluskicide includes 2-(thiocyanomethyl-thio)benzothiozole (CAS No. 21564-17-0) and methylene bis(thiocyanate) (CAS No. 6317-18-6).

In certain embodiments an environmental law or regulation may encourage the selection of an organic biocide such as a benzisothiazolinone derivative. An example of a benzisothiazolinone derivative is Busan 1264 (Buckman Laboratories, Inc.), Proxel™ GXL, Proxel™ TN, Proxel™ XL2, Proxel™ BD20 and Proxel™ BZ (Avecia Inc.), Preventol® VP OC 3068 (Bayer Corporation), or Mergal® K10N (Troy Corp.) which comprises 1,2-benzisothiazoline-3-one (CAS No. 2634-33-5). In the case of Busan™ 1264, the primary use is a bactericide and/or fungicide at 0.03% to 0.5% in a water-borne coating. Proxel™ TN comprises 1,2-benzisothiazoline-3-one (CAS No. 2634-33-5) and hexahydro-1,3,5-tris(2-hydroxyethyl)-s-triazine (“triazine”; CAS No. 4719-04-4), Proxel™ GXL, Proxel™ XL2 and Proxel™ BD20 comprises 1,2-benzisothiazoline-3-one (CAS No. 2634-33-5), Proxel™ BZ comprises 1,2-benzisothiazoline-3-one (CAS No. 2634-33-5) and zinc pyrithione (CAS No. 13463-41-7), and are typically used in industrial coatings and water-based coatings as a bactericide/fungicide. Mergal® KION comprises 1,2-benzisothiazoline-3-one (CAS No. 2634-33-5), and is typically used in water-borne coatings as a bactericide/fungicide.

Often, a preservative is a proprietary commercial formulation and/or a compound sold under a tradename. Examples include organic biocides under the tradename Nuosept® (International Specialty Products), which are typically used in a water-borne coating. Specific examples of a Nuosept® biocide includes Nuosept® 95, which comprises a mixture of bicyclic oxazolidines, and is typically added to 0.2% to 0.3% concentration to a coating composition; Nuosept® 145, which comprises an amine reaction product, and is typically added to 0.2% to 0.3% concentration to a coating composition; Nuosept® 166, which comprises 4,4-dimethyloxazolidine (CAS No. 51200-87-4), and is typically added to 0.2% to 0.3% concentration to a basic pH water-borne coating composition; or a combination thereof. A further example is Nuocide® (International Specialty Products) biocides, which are typically used fungicides and/or algaecides. Examples of a Nuocide® biocide is Nuocide® 960, which comprises 96% tetrachlorisophthalonitrile (CAS No. 1897-45-6), and is typically used at 0.5% to 1.2% in a water-borne or solvent-borne coating as a fungicide; Nuocide® 2010, which comprises chlorothalonil (CAS No. 1897-45-6) and IPBC (CAS No. 55406-53-6) at 30%, and is typically used at 0.5% to 2.5% in a coating as a fungicide and algaecide; Nuocide® 1051 and Nuocide® 1071, each which comprises 96% N-cyclopropyl-N-(1-dimethylethyl)-6-(methylthio)-1,3,5-triazine-2,4-diamine (CAS No. 28159-98-0), and is typically used as an algaecide in antifouling coatings at 1.0% to 6.0% or water-based coatings at 0.05% to 0.2%, respectively; and Nuocide® 2002, which comprises chlorothalonil (CAS No. 1897-45-6) and a triazine compound at 30%, and is typically used at 0.5% to 2.5% in a coating and/or a film as a fungicide and algaecide.

An additional example of a tradename biocide for coatings includes Vancide® (R. T. Vanderbilt Company, Inc.). Examples of a Vancide® biocide include Vancide® TH, which comprises hexahydro-1,3,5-triethyl-s-triazine (CAS No. 108-74-7), and is generally used in a water-borne coating; Vancide® 89, which comprises N-trichloromethylthio-4-cyclohexene-1,2-dicarboximide (CAS No. 133-06-2) and related compounds such as captan (CAS No. 133-06-2), and is used as a fungicide in a coating composition; or a combination thereof. A bactericide and/or fungicide for coatings, particularly a water-borne coating, is a Dowicil™ (Dow Chemical Company). Examples of a Dowicil™ biocide include Dowicil™ QK-20, which comprises 2,2-dibromo-3-nitrilopropionamide (CAS No. 10222-01-2), and is used as a bactericide at 100 ppm to 2000 ppm in a coating; Dowicil™ 75, which comprises 1-(3-chloroallyl)-3,5,7-triaza-1-azoniaadamantane chloride (CAS No. 51229-78-8), and is used as a bactericide at 500 ppm to 1500 ppm in a coating; Dowicil™ 96, which comprises 7-ethyl bicyclooxazolidine (CAS No. 7747-35-5), and is used as a bactericide at 1000 ppm to 2500 ppm in a coating; Bioban™ CS-1135, which comprises 4,4-dimethyloxazolidine (CAS No. 51200-87-4), and is used as a bactericide at 100 ppm to 500 ppm in a coating; or a combination thereof. An additional example of a tradename biocide for coatings includes Kathon® (Rohm and Haas Company). An example of a Kathon® biocide includes Kathon® LX, which typically comprises 5-chloro-2-methyl-4-isothiazolin-3-one (CAS no 26172-55-4) and 2-methyl-4-isothiazolin-3-one (CAS no 2682-20-4) at 1.5%, and is added from 0.05% to 0.15% in a coating. Examples of tradename fungicides and algaecides include those described for Fungitrol® and Biotrend® (International Specialty Products), which are often formulated for solvent-borne and water-borne coatings, and in-can and film preservation. An example is Fungitrol® 158, which comprises 15% tributyltin benzoate (CAS No. 4342-36-3) (15%) and 21.2% alkylamine hydrochlorides, and is typically used at 0.35% to 0.75% in a water-borne coating for in-can and film preservation. An additional example is Fungitrol® 11, which comprises N-(trichloromethylthio) phthalimide (CAS No. 133-07-3), and is typically used at 0.5% to 1.0% as a fungicide for solvent-borne coating. A further example is Fungitrol® 400, which comprises 98% 3-iodo-2-propynl N-butyl carbamate (“IPBC”) (Cas No. 55406-53-6), and is typically used at 0.15% to 0.45% as a fungicide for a water-borne or a solvent-borne coating.

Further examples of a tradename biocide for coatings includes various Omadine® or Triadine® products (Arch chemicals, Inc.), Densil™ P, Densil™ C404, Densil™ DN, Densil™ DG20 and Vantocil™ IB (Avecia Inc.), Polyphase® 678, Polyphase® 663, Polyphase® CST, Polyphase® 641, Troysan® 680 (Troy Corp.), Rocima® 550, Rocima® 607, Rozone® 2000 and Skane™ M-8 (Rohm and Haas Company) and Myacide™ GDA, Myacide™ GA 15, Myacide™ Ga 26, Myacide™ 45, Myacide™ AS Technical, Myacide™ AS 2, Myacide AS 30, Myacide™ AS15, Protectol™ PE, Daomet™ Technical and Myacide™ HT Technical (BASF Corp.). Zinc Omadine® (“zinc pyrithione”; CAS No. 13463-41-7) is a fungicide/algaecide typically used as an in-film preservative and/or anti-fouling preservative; sodium Omadine® (“sodium pyrithione”; CAS No. 3811-73-2) is typically used as a fungicide/algaecide in-film preservative; copper Omadine® (“copper pyrithione”; CAS No. 14915-37-8) is typically used as a fungicide/algaecide in-film preservative and/or anti-fouling preservative; Triadine® 174 (“triazine,” “1,3,5-triazine-(2H,4H,6H)-triethanol”; “hexahydro-1,3,5-tris(2-hydroxyethyl)-s-triazine “; Cas No. 4719-04-4) is a bacteria biostatic/bactericide typically used in water-borne coatings; Densil™ P comprises dithio-2,2-bis(benzmethylamide) (CAS No. 2527-58-4) and is typically used in industrial coatings, water-based coatings and films thereof as a fungicide/bactericide; Densil™ C404 comprises 2,4,5,6-tetrachloroisophthalonitrile (“chlorothalonil”; CAS No. 1897-45-6) and is used as a fungicide; Densil™ DN and Densil™ DG20 comprise N-butyl-1,2-benzisothiazolin-3-one (CAS No. 4299-07-4), and each may be used as a fungicide; Vantocil™ IB comprises poly(hexamethylene biguanide) hydrochloride (CAS No. 27083-27-8) and is a micobiocide; Polyphase® 678 comprises carbendazim (CAS No. 10605-21-7) and 3-iodo-2-propynyl butyl carbamate (CAS No. 55406-53-6) and is typically used as an antimicrobial biocide for exterior coatings and surface treatments; Polyphase® 663 comprises 3-iodo-2-propynyl butyl carbamate (CAS No. 55406-53-6), carbendazim (CAS No. 10605-21-7) and diuron (CAS No. 330-54-1) and is typically used as a fungicide/algaecide in exterior coatings; Rocima® 550 comprises 2-methyl-4-isothiazolin-3-one (CAS No. 2682-20-4), and is typically used as a bactericide/fungicide for water-borne coatings; Rozone® 2000 comprises 4,5-dichloro-2-N-octyl-3(2H)-isothiazolone (CAS No. 64359-81-5) and is used as a microbiocide for latex coatings; Skane™ M-8 comprises 2-Octyl-4-isothiazolin-3-one (CAS No. 26530-20-1), and may be used as an in-film fungicide; Myacide™ GDA Technical, Myacide™ GA 15, Myacide™ Ga 26 and Myacide™ 45 each comprise glutaraldehyde (CAS No. 111-30-8) and are typically used as an algaecide/bactericide/fungicide; Myacide™ AS Technical, Myacide™ AS 2, Myacide™ AS 30, Myacide™ AS15 each comprise 2-bromo-2-nitropropane-1,3-diol (“bronopol”; Cas No. 52-51-7) and are typically used as an algaecide; Protectol™ PE comprises phenoxyethanol (CAS No. 122-99-6) and can be used as microbiocide/fungicide; Dazomet™ Technical comprises 3,5-dimethyl-2H-1,3,5-thiadiazinane-2-thione (“dazomet”; CAS No. 533-74-4) and may be used as a microbiocide/fungicide; Myacide™ HT Technical comprises 1,3,5-tris-(2-hydroxyethyl)-1,3,5-hexahydrotriazine (CAS No. 4719-04-4) and can be used as a microbiocide/fungicide.

Determination of whether damage to a coating and/or film is due to microorganisms (e.g., film algal defacement, film fungal defacement), as well as the efficacy of addition of a preservative to a coating and/or film composition in reducing microbial damage to a coating and/or film, may be empirically determined by techniques such as those that are described in “ASTM Book of Standards, Volume 06.01, Paint—Tests for Chemical, Physical, and Optical Properties; Appearance,” D3274-95, D4610-98, D2574-00, D3273-00, D3456-86, D5589-97, and D5590-00, 2002; and in “Paint and Coating Testing Manual, Fourteenth Edition of the Gardner-Sward Handbook,” (Koleske, J. V. Ed.), pp. 654-661, 1995. Examples of microorganisms typically selected in such procedures as positive controls of a coating and/or film damaging microorganism include, for example, Aspergillus oryzae (ATCC 10196), Aspergillus flavus (ATCC 9643), Aspergillus niger (ATCC 9642), Pseudomonas aeruginosa (ATCC 10145), Aureobasidium pullulans (ATCC 9348), Penicillium citrinum (ATCC 9849), Penicillium funiculosum (ATCC 9644), or a combination thereof.

b. Wetting Additives and Dispersants

It is contemplated that one or more types of particulate matter (e.g., a pigment, a cell-based particulate material) may be incorporated into a coating composition. Physical force and/or chemical additives are used to promote a desirable level of dispersion of particulate matter in a coating composition, for purposes such as coating homogeneity and ease of application. Depending upon whether such an additive is admixed earlier or latter in a coating composition, such an additive is known as a wetting agent or a dispersant, respectively, though it is common that an additive has dual classification. A wetting agent and/or a dispersant often can be used to reduce the particulate matter grinding time during coating preparation, improve wetting of particulate matter, improve dispersion of particulate matter, improve gloss, improve leveling, reduce flooding, reduce floating, reduce viscosity, reduce thixotropy, or a combination thereof.

It is contemplated that in certain embodiments, a biomolecular composition (e.g., a cell-based particulate material) may be used as a wetting additive and/or dispersant. Though this use may be conter-intuitive, it is contemplated that embodiments such as a cell-based particulate material may promote the separation of particulate material (e.g., a pigment, an additional preparation of a cell-based particulate material) by acting as a physical barrier between particles of particulate material. It is further contemplated that in embodiments wherein the cell-based particulate material is used as a wetting additive and/or dispersant, it may, of course, be combined with a traditional wetting additive and/or dispersant, examples of which are described below.

(1) Wetting Additives

Preparation of a Coating Comprising Particulate Material Often Comprises a Step Wherein the particulate material is dispersed in an additional coating component. An example of this type of dispersion step is the dispersion of a pigment into a combination of a liquid component and a binder to form a material known as a millbase. A wetting additive (“wetting agent”) is a composition added to promote dispersion of particulate material during coating preparation.

In certain embodiments, a wetting agent is a molecule that comprises a polar region and a nonpolar region. An example is an ethylene oxide molecule comprising a hydrophobic moiety. Such a wetting agent is thought to act by reducing interfacial tension between a liquid component and particulate matter. In specific aspects, a wetting agent comprises a surfactant. Examples of such a wetting agent include pine oil, which is typically added at 1% to 5% of the total coating liquid component, including all intermediate ranges and combinations thereof. Other examples of wetting agents include a metal soap, such as, for example, calcium octoate, zinc octoate, aluminum stearate, zinc stearate, or a combination thereof. An additional example of a wetting agent is bis(2-ethylhexyl)sulfosuccinate (“Aerosol OT”) (Cas No. 577-11-7); (octylphenoxy)polyethoxyethanol octylphenyl-polyethylene glycol (“Igepal-630”) (Cas no. 9036-19-5); nonyl phenoxy poly (ethylene oxy)ethanol (“Tergitol NP-14”) (Cas No. 9016-45-9); ethylene glycol octyl phenyl ether (“Triton X-100”) (CAS No. 9002-93-1); or a combination thereof.

Often a wetting agent and/or dispersant is a proprietary formulation and/or commonly available under a trade name. Examples include an Anti-Terra® or Disperbyk® (BYK-Chemie GmbH) and EnviroGem® or Surfynol® (Air Products and Chemicals, Inc.) wetting agents and/or dispersants. An example is Anti-Terra®-U, which comprises a 50% solution of an unsaturated polyamine amide salt and a lower molecular weight acid, dissolved in xylene and isobutanol, and may be selected for used in a solvent-borne coating. Anti-Terra®-U is typically added from 1% to 2% to an inorganic pigment, 1% to 5% to an organic pigment, and at 0.5% to 1.0% to titanium dioxide, and 30% to 50% to a bentonite. An example of a Disperbyk® is Disperbyk®, which comprises a polycarboxylic acid polymer alkylolammonium salt and water, and is added to 0.3% to 1.5% to the solvent-borne or water-borne coating composition. A further example is Disperbyk®-101, which comprises a 52% solution of a long chain polyamine amide salt and a polar acidic ester, dissolved in a mineral spirit and butylglycol, and may be used in a solvent-borne coating. The ranges for addition to particulate material for Disperbyk®-101 is similar to Anti-Terra®-U. An additional example is Disperbyk®-108, which comprises over 97% of a hydroxyfunctional carboxylic acid ester that includes moieties with pigment affinity, and is typically added from 3% to 5% to an inorganic pigment, 5% to 8% to an organic pigment. However, Disperbyk®-108 is typically added at 0.8% to 1.5% to titanium dioxide, or 8% to 10% to a carbon black, and may be used for coatings lacking a non-aqueous solvent. A supplemental example is EnviroGem® AD01, which comprises a non-ionic wetting agent with a defoaming property, and is added to 0.1% to 2% to a water-borne coating composition. An additional example is Surfynol® TG (Air Products and Chemicals, Inc.), which comprises a non-ionic wetting agent, and is added to 0.5% to 5% to a water-borne coating composition. A further example is Surfynol® 104 (Air Products and Chemicals, Inc.), which comprises a non-ionic wetting agent, dispersant, and defoamer, and is added to 0.05% to 3% to a water-borne coating composition.

(2) Dispersants

Maintenance of the dispersal of particulate matter comprised within a coating composition is often promoted by the addition of a dispersant. A dispersant (“dispersing additive,” “deflocculant,” “antisettling agent”) is a composition that is added to promote continuing dispersal of particulate matter. In specific aspects, a dispersant is added to a coating composition to reduce or prevent flocculation. Flocculation is the process wherein a plurality of primary particles that have been previously dispersed form an agglomerate. In other aspects, a dispersant is added to a coating composition to prevent sedimentation of particulate matter. Standard procedures to determining the degree of settling by particulate matter in a coating (e.g., paint) are described, for example, in “ASTM Book of Standards, Volume 06.02, Paint—Products and Applications; Protective Coatings; Pipeline Coatings,” D869-85, 2002.

Often a dispersant is a compound comprising phosphate, such as, for example, tetra-potassium pyrophosphate or “TKPP” (CAS No. 7320-34-5). Examples of a tradename/proprietary phosphate compounds are those known as a Strodex™ (Dexter Chemical L.L.C.), including Strodex™ PK-90, Strodex™ PK-0VOC, and/or Strodex™ MOK-70, which comprise a phosphate ester surfactant.

In some aspects, a dispersant may be a particulate material. Examples include Winnofil® SPT Premium, Winnofil® S, Winnofil® SPM, and Winnofil® SPT (Solvay Advanced Functional Minerals), which comprise 97.4% calcium carbonate (CAS No. 471-34-1) coated with 2.6% fatty acid (CAS No. 64755-01-7) and generally used at 2% to 3%.

Various preparations of modified montmorillonite clay are known in the art as a dispersant. Examples include those under the name Bentone® (Elementis Specialties, lnc). Bentone® 34 (Elementis Specialties, Inc), which comprises tetraallyl ammonium bentonite, and is prepared with 33% or more polar solvent prior to addition to a coating composition. M-P-A® 14 (Elementis Specialties, Inc.), which comprises a montmorillonite clay modified by and organic chemical, and is prepared with 33% or more polar solvent prior to addition to a solvent-borne coating composition. Bentone® SD-1 (Elementis Specialties, Inc.), which comprises a montmorillonite clay modified by and organic chemical, and typically added from 0.2% to 2% by weight to a solvent-borne coating composition, particularly those comprising an aliphatic liquid component.

A further example of a dispersant is a castor wax formulation under the trade names Crayvallac® SF, Crayvallac® MT, and Crayvallac® AntiSettle CVP (Cray Valley Limited), each of which are typically added from 0.2% to 1.5% as a dispersant, thixotropy additive, anti-sagging agent, or a combination thereof. Crayvallac® AntiSettle CVP comprises caster wax (“hydrogenated caster oil”), and is suitable for a solvent free epoxy-coating and a mineral spirit liquid component. Crayvallac® SF and Crayvallac® MT each comprise amide modified caster wax, and may be used in an epoxy-coating, an acrylic-coating, a chlorinated rubber-coating, or a combination thereof. Crayvallac® SF and Crayvallac® MT may be used with a liquid component comprising an aromatic hydrocarbon, an alcohol, a glycol ether, or a combination thereof with Crayvallac® MT being also may be used with a mineral spirit.

c. Buffers

In certain embodiments, it is desirable to maintain a coating's pH within a certain range. The pH may range from 0 to 14, including all intermediate ranges and combinations thereof. A coating may be acidic, which is a pH between 0 and 7, including all intermediate ranges and combinations thereof, or basic, which is a pH between 7 and 14, including all intermediate ranges and combinations thereof. A neutral pH is pH 7.0, and it is contemplated that a coating may have a neutral pH, or a pH that is near neutral, which is a pH between 6.5 and 7.5, including all intermediate ranges and combinations thereof. A buffer may be added to maintain a coating's pH in a desired range, such as, for example, acidic, basic, neutral, or near neutral. In some embodiments, the pH buffer is selected to help maintain a coating or surface treatment's pH to promote the activity of a biomolecular composition, such as an enzyme's activity. For example, in certain aspects, a basic pH may improve the function of an enzyme, such as, for example, a lipolytic enzyme or OPH that functions better in basic pH range. For example, the acids released by a lipolytic ezyme's activity may detrimentally alter the local pH relative to optimum conditions for activity, and a buffer may reduce this effect. Alternatively, the buffer may be selected for biomolecular compositions that function at neutral or basic pH, or to effect the function of other coating or surface treatment components, such as, for example, the curing process. Examples of buffers include a bicarbonate (e.g., an ammonium bicarbonate), a monobasic phosphate buffer, a dibasic phosphate buffer, Trizma base, a 5 zwitterionic buffer, triethanolamine, or a combination thereof. In particular facets, a buffer such as a bicarbonate, may provide a ligand or co-substrate (e.g., water) on activator (e.g., carbon dioxide) to an enzyme to promote an enzymatic reaction. In particular facets, it is contemplated that a buffer will comprise 0.000001 M to 2.0 M, including all intermediate ranges and combinations thereof, in a coating or other surface treatment.

d. Rheology Modifiers

A rheology modifier (“rheology control agent,” “rheology additive,” “thickener and rheology modifier,” “TRM,” “Theological and viscosity control agent,” “viscosifier,” “viscosity control agent,” “thickener”) is a composition that alters (e.g., increases, decreases, maintains) a rheological property of a coating. A thickener (“thickening agent”) increases and/or maintains viscosity. A rheological property is a property of flow and/or deformation. Examples of a rheological property include viscosity, brushability, leveling, sagging, or a combination thereof. Viscosity is a measure of a fluid's resistance to flow (e.g., a shear force). Brushability is the ease a coating can be applied using an applicator (e.g., a brush). Leveling is the ability of a coating to flow into and fill uneven areas of coating thickness (e.g., brush marks) after application to a surface and before sufficient film formation to end such flow. Sagging is the gravitationally induced downward flow of a coating after application to a surface and before sufficient film formation to end such flow. It is specifically contemplated that a cell-based particulate material may be added to a coating as a rheology modifier. It is further contemplated that in embodiments wherein the cell-based particulate material is used as a rheology modifier, it may, of course, be combined with a traditional rheology modifier, examples of which are described below.

A rheology modifier that alters viscosity (e.g., increases, decreases, maintains) is known as a “viscosifier.” During preparation, the viscosity of a coating (“medium-shear viscosity,” “mid-shear viscosity,” “coating consistency”) is often measured to verify a viscosity that is often suitable for a coating during storage, application, etc. The typical range of shear force for measuring mid-shear viscosity is between 10 s⁻¹ to 10³ s⁻¹. In many embodiments, particularly for architectural coatings, a medium shear viscosity will be between 60 Ku and 140 Ku, including all intermediate ranges and combinations thereof. During application (“high-shear”), a coating is usually subjected to a shear force 10³ s⁻¹ to 10⁴ s⁻¹ by techniques such as brush application, and a shear force up to or greater than 10⁶ s⁻¹ by techniques including, for example, blade application, high-speed roller application, spray application, or a combination thereof. A coating typically is formulated to possess a viscosity upon the shear force of application (“high-shear viscosity”) that promotes the ease of application. An example of a high shear viscosity during application is between 0.5 P (“50 mPa s”) to 2.5 P (“250 mPa s”), including all intermediate ranges and combinations thereof. In certain aspects, a coating may possess a viscosity greater or lower than this range, however, it is contemplated such a viscosity may make the coating more difficult to apply using the above application techniques. Post-preparation and/or post-application, a coating is usually subjected to a shear force of 10 s⁻¹ to 10⁻³ s⁻¹ produced, for example, by forces such as gravity, capillary pressure, or a combination thereof. In embodiments wherein a coating's viscosity (“low-shear viscosity”) is too high at these levels of shear force (“low-shear”), leveling during and/or after application may be undesirably low. In embodiments wherein a viscosity is too low at these levels of shear force, a coating may suffer in-can settling, sagging during or after application, or a combination thereof. In some embodiments, viscosity of a coating post-preparation and/or application is between 100 P (“10 Pa s”) to 1000 P (“100 Pa s”), the including all intermediate ranges and combinations thereof. In other aspects, the coating has a viscosity of 100 P to 1000 P, including all intermediate ranges and combinations thereof, upon a surface immediately after application. In some embodiments, the viscosity of the coating varies during preparation (“mixing”), during storage (e.g., in a container), during application, and upon a surface. The medium-shear viscosity (“coating consistency”) refers to the viscosity of a coating during preparation, and in most embodiments will be between 60 Ku and 140 Ku, including all intermediate ranges and combinations thereof. Specific examples of medium-shear viscosity intermediate ranges and combinations thereof include 70 Ku to 110 Ku, 80 Ku to 100 Ku, 90 Ku to 95 Ku, 72 Ku to 95 Ku, etc. During storage and upon a surface, a coating is typically subject to lower shear forces (e.g., gravity), and a coating may possess a viscosity and other rheological properties (e.g., leveling, sag, syneresis, settling) to retain suitable dispersion of coating components during storage and form a uniform layer upon a surface. It is contemplate that in most embodiments, the low-shear viscosity (e.g., the viscosity prior to application, viscosity upon a surface immediately after application) of a coating will be between 100 P to 3000 P, including all intermediate ranges and combinations thereof. Specific examples of low-shear viscosity intermediate ranges and combinations thereof include 100 P to 2500 P, 100 P to 2000 P, 100 P to 1500 P, 100 P to 1000 P, 125P to 3000 P, 150P to 3000 P, 175P to 3000P, 200P to 3000 P, 225P to 3000 P, 250P to 3000P, 275P to 3000 P, 300P to 3000 P, 125P to 2500 P, 150P to 2000 P, 175P to 1500 P, 200P to 1000 P, 250 P to 1000 P, etc. The high-shear viscosity (“application viscosity”) refers to the viscosity of a coating during application, and typically is less than the low-shear viscosity to allow ease of application. In particular aspects, the coating has a high-shear viscosity of 0.5 P to 2.5 P, including all intermediate ranges and combinations thereof. Specific examples of high-shear viscosity intermediate ranges and combinations thereof include 0.5 P to 2.0 P, 0.5 P to 1.5 P, 0.5 P to 1.0 P, 0.5 P to 0.75 P, 0.6 P to 2.5 P, 0.75 P to 2.5 P, 1.0 P to 2.5 P, 1.5 P to 2.5 P, 2.0 P to 2.5 P, 0.75 P to 2.0 P, 1.0 P to 2.0 P, etc. Of course, the viscosity of a coating will change post-application in embodiments wherein film formation occurs; however, the post-application viscosity refers to the viscosity prior to completion of film formation, and may be determined immediately post-application (e.g., within seconds, within minutes) as appropriate to the coating, using technique in the art. In certain aspects, a coating may possess a viscosity greater or lower than this range, however, it is contemplated such a viscosity may make the coating more prone to sagging and/or settling defects. Techniques for measuring viscosity (e.g., low-shear viscosity, medium-shear viscosity, high-shear viscosity) are known to those of skill in the art [see, for example, “ASTM Book of Standards, Volume 06.01, Paint—Tests for Chemical, Physical, and Optical Properties; Appearance,” D562-01, D2196-99, D4287-00, 2002; and in “Paint and Coating Testing Manual, Fourteenth Edition of the Gardner-Sward Handbook,” (Koleske, J. V. Ed.), 1995].

A rheology modifier is typically added to alter and/or maintain a rheology property within a desired range post-formulation, during application, post-application, or a combination thereof. In specific embodiments, a rheology modifier alters viscosity at or above 103 s⁻¹ and/or at or below 10 s⁻¹. Viscosity, including non-Newtonian (e.g., shear-thinning) viscosity for coatings and/or coating components (e.g., binders, binder solutions, vehicles) upon formulation with or without a viscosity modifier can be empirically determined, particularly for shear rates comparable to application techniques (e.g., blade, brush, roller, spray) by standard techniques such as in “ASTM Book of Standards, Volume 06.01, Paint—Tests for Chemical, Physical, and Optical Properties; Appearance,” D562-01, D2196-99, D4287-00, D4212-99, D1200-94, D5125-97, and D5478-98, 2002; “ASTM Book of Standards, Volume 06.02, Paint—Products and Applications; Protective Coatings; Pipeline Coatings,” D4958-97, 2002; and “ASTM Book of Standards, Volume 06.03, Paint—Pigments, Drying Oils, Polymers, Resins, Naval Stores, Cellulosic Esters, and Ink Vehicles,” D1545-98, D1725-62, D6606-00 and D6267-98, 2002. Additionally, other rheological properties can be determined to aid formulation of a coating using techniques in the art. For example, brush drag, which is the resistance during coating (e.g., a latex) application using a brush, can be determined by standard techniques, such as, for example, in “ASTM Book of Standards, Volume 06.02, Paint—Products and Applications; Protective Coatings; Pipeline Coatings,” D4040-99, 2002. In an additional example, leveling and sagging can be empirically determined for a coating by standard techniques such as in “ASTM Book of Standards, Volume 06.02, Paint—Products and Applications; Protective Coatings; Pipeline Coatings,” D4062-99 and D4400-99, 2002.

The addition of a coating component to a coating composition typically alters a rheological property, and many coating components have multiple classifications to include function as a rheology modifier. Examples of coating components more commonly added for function as a rheology modifier include an inorganic rheology modifier, an organometallic rheology modifier, an organic rheology modifier, or a combination thereof. An example of an inorganic rheology modifier includes a silicate such as a montmorillonite silicate. An example of a montomorillonite silicate includes aluminum silicate, a bentonite, magnesium silicate, or a combination thereof. A silicate rheology modifier typically confers a superior washfastness property, a superior abrasion resistance property, or a combination thereof, to a coating relative to an organic rheology modifier. An example of an organic rheology modifier includes a cellulose ether, a hydrogenated oil, a polyacrylate, a polyvinylpyrrolidone, a urethane, or a combination thereof. Organic rheology modifiers of a polymeric nature (e.g., a cellulose ether, a urethane, a polyacrylate, etc.) are sometimes used as an associative thickener, and may be used for a latex coating. An organic rheology modifier typically confers a greater water retention capacity property (“open time”) to a coating relative to a silicate rheology modifier. A common example of a cellulose ether is a methyl cellulose, a hydroxyethyl cellulose, or a combination thereof. An example of a hydroxyethyl cellulose includes Natrosol® (Hercules Incorporated); Cellosize™ (Dow Chemical Company); or a combination thereof. An example of hydrogenated oil includes hydrogenated castor oil. An example of a urethane rheology modifier (“associative thickener”) includes a hydrophobically modified ethylene oxide urethane (“HEUR”), which comprises a polyethylene glycol block covalently linked by urethane, and has both a hydrophilic and hydrophobic regions capable of use in an aqueous environment. An example of a HEUR includes a block of polyethylene oxide linked by a urethane and modified with a nonyl phenol hydrophobe (Rohm and Haas Company). Often a urethane rheology modifier confers a superior leveling property over another type of organic rheology modifier. An example of an organometallic rheology modifier includes a titanium chelate, a zirconium chelate, or a combination thereof.

In addition to the disclosures herein, a rheology modifier and use of a rheology modifier in a coating is known to those of skill in the art, and such compositions and techniques may be included (see, for example, Flick, E. W. “Handbook of Paint Raw Materials, Second Edition,” 808-843 and 879-998, 1989; in “Paint and Coating Testing Manual, Fourteenth Edition of the Gardner-Sward Handbook,” (Koleske, J. V. Ed.), pp 268-285 and 348-349, 1995; in “Paint and Surface Coatings, Theory and Practice, Second Edition,” (Lambourne, R. and Strivens, T. A., Eds.), pp. 73, 218, 227, 352, 558-559 and 718, 1999; Wicks, Jr., Z. W., Jones, F. N., Pappas, S. P. “Organic Coatings, Science and Technology, Volume 2: Applications, Properties and Performance,” pp. 42, 215, 293, 315, 320 and 323-328, 1992; and in “Paints, Coatings and Solvents, Second, Completely Revised Edition,” (Stoye, D. and Freitag, W., Eds.) pp 6, 128 and 166-167, 1998.

e. Defoamers

A coating sometimes comprises a gas capable of forming a bubble (“foam”) that can undesirably alter a physical and/or aesthetic property. Undesirable gas incorporation into a coating composition is often a side effect of coating preparation processes, and a particular bane of latex coatings. Often, a wetting agent and/or a dispersant used in a coating may promote creation or retention of foam. Additionally, cells (e.g., microorganisms) can produce gas, and in certain embodiments, a coating comprising a cell-based particulate material may also comprise a defoamer. A defoamer (“antifoaming agent,” “antifoaming additive”) is a composition that releases gas (e.g., air) and/or reduces foaming in a coating during production, application, film formation, or a combination thereof. A defoamer often acts by lowering the surface tension around a bubble, allowing merging of a bubble with a second bubble, which produces a larger and less stable bubble that collapses. It is contemplated that in certain coating compositions, a cell-based particulate material may act as a defoamer by destabilizing a bubble in a coating. It is further contemplated that in embodiments wherein the cell-based particulate material is used as a defoamer, it may, of course, be combined with a traditional defoamer, examples of which are described below.

Examples of a defoamer include an oil (e.g., a mineral oil, a silicon oil), a fatty acid ester, dibutyl phosphate, a metallic soap, a siloxane, a wax, an alcohol comprising between six to ten carbons, or a combination thereof. An example of an oil defoamer is pine oil. In some aspects, an antifoaming agent is combined with an emulsifier, a hydrophobic silica, or a combination thereof. Examples of a tradename defoamer is a TEGO® Foamex 8050 (Goldschmidt Chemical Corp.), which comprises a polyether siloxane copolymer and fumed silica, and typically is used at 0.1% to 0.5% during coating preparation; and BYK®-31 (BYK-Chemie), which comprises a paraffin mineral oil and hydrophobic compounds, and typically is used at 0.1% to 0.5% in a coating.

f. Catalysts

A catalyst is an additive that promotes film formation by catalyzing a cross-linking reaction in a thermosetting coating. Examples of a catalyst include a drier, an acid or a base, and the selection of the type of catalyst is specific to the chemistry of the film formation reaction.

(1) Driers

A drier (“siccative”) catalyzes is an oxidative film formation reaction, such as those that occur in an oil-based coating. In addition to the disclosures herein, an drier and use of a drier in a coating is known to those of skill in the art, and such materials and techniques for using a drier in a coating may be used (see, for example, Flick, E. W. “Handbook of Paint Raw Materials, Second Edition,” pp. 73-93 and 879-998, 1989; in “Paint and Coating Testing Manual, Fourteenth Edition of the Gardner-Sward Handbook,” (Koleske, J. V. Ed.), pp 30-35, 1995; in “Paint and Surface Coatings, Theory and Practice, Second Edition,” (Lambourne, R. and Strivens, T. A., Eds.), pp. 190-192, 1999; Wicks, Jr., Z. W., Jones, F. N., Pappas, S. P. “Organic Coatings, Science and Technology, Volume 1: Film Formation, Components, and Appearance,” pp. 138, 317-318, 1992; Wicks, Jr., Z. W., Jones, F. N., Pappas, S. P. “Organic Coatings, Science and Technology, Volume 2: Applications, Properties and Performance” pp. 138, 197-198, 330, 344, 1992; and in “Paints, Coatings and Solvents, Second, Completely Revised Edition,” (Stoye, D. and Freitag, W., Eds.) pp. 11, 48, 165, 1998.

A drier may comprise a metal drier, an alternative drier, a feeder drier, or a combination thereof. Usually a drier comprising a metal (“a metal drier”) catalyzes the oxidative reaction. Examples of a metal typically used in a drier includes aluminum, barium, bismuth, calcium, cerium, cobalt, iron, lanthanum, lead, manganese, neodymium, potassium, vanadium, zinc, zirconium, or a combination thereof. Examples of types of metal driers include an inorganic metal salt, a metal-organic acid salt (“soap”), or a combination thereof. A “salt” is the composition formed between the anion of an acid and the cation of a base. Typically, the acid and base of a salt interact by an ionic bond. Examples of organic acids used in such a soap include a monocarboxylic acid of 7 to 22 carbon atoms. Examples of such a monocarboxylic acid include a linoleate, a naphthenate, a neodecanoate, an octoate, a rosin, a synthetic acid, a tallate, or a combination thereof. Examples of a drier comprising a synthetic acid include those under the tradenames Troymax™ (Troy Corporation). Though many driers are water insoluble, water dispersible driers can be prepared by combining a surfactant with a naphthenate drier and/or a synthetic acid drier. However, water dispersible driers are typically obtained under a tradename such as, for example, Troykyd® Calcium WD, Troykyd® Cobalt WD, Troykyd® Manganese WD Troykyd® Zirconium WD (Troy Corporation). Additionally, a potassium soap, lithium soap, or a combination thereof, has limited aqueous solubility.

A primary drier (“surface drier,” “active drier,” “top drier”) acts at the coating-external environment interface. A secondary drier (“auxiliary drier,” “through drier”) acts throughout the coating. Examples of primary driers include metal driers comprising cobalt, manganese, vanadium, or a combination thereof. Examples of secondary driers include metal driers comprising aluminum, barium, calcium, cerium, iron, lanthanum, lead, manganese, neodymium, zinc, zirconium, or a combination thereof. A rare earth drier comprises lanthanum, neodymium, cerium, or a combination thereof.

In many embodiments, it is contemplated that a coating will comprise from 0.01% to 0.1%, including all intermediate ranges and combinations thereof, of an individual metal of a primary drier, by weight of the non-volatile components of a coating composition. In many embodiments, it is contemplated that a coating will comprise from 0.1% to 1.0%, including all intermediate ranges and combinations thereof, of an individual metal of a secondary drier, by weight of the non-volatile components of a coating composition. Standard physical and/or chemical properties for various driers comprising a metal (e.g., calcium, cerium, cobalt, iron, lead, manganese, nickel, rare earth, zinc, zirconium), and procedures for determining various metals' content for a driers are described in, for example, “ASTM Book of Standards, Volume 06.04, Paint—Solvents; Aromatic Hydrocarbons,” D600-90, 2002; and “Volume 06.01, Paint—Tests for Chemical, Physical, and Optical Properties; Appearance,” D2373-85, D2374-85, D2375-85, D2613-01, D3804-02, D3969-01, D3970-80, D3988-85, and D3989-01, 2002; and ASTM Book of Standards, Volume 06.01, Paint—Tests for Chemical, Physical, and Optical Properties; Appearance,” D564-87, 2002.

In embodiments wherein a secondary drier is used, it may be combined with a primary drier, as the activity of many secondary driers are often limited when acting without the presence of a primary drier. Skinning is film-formation disproportionately at the coating-external environment interface. Skinning often results in undesirable wrinkle formation (“wrinkling”) in the film. A primary drier undesirably promotes skinning when acting without the presence of a secondary drier. In certain aspects, zinc may be selected for reducing wrinkling in thick films. In other aspects, calcium and/or zirconium may be selected instead of lead, which may be limited due to an environmental law or regulation. In some facets, an iron drier, rare earth drier, or combination thereof, may be selected for use during film formation by baking. However, an iron drier may darken a coating. In further aspects, an aluminum drier may be selected for an alkyd-coating.

An alternative drier is a type of drier developed for use in a high solid and/or water-borne coating, due to the inefficiency of a metal-soap drier in these types of coatings. Often, an alternative drier is combined with a metal-soap drier. An example of a metal soap drier include a 1,10-phenanthronine, 2,2′-dipyridyl. A feeder drier is a type of drier designed to prolong the pot life of a coating in embodiments wherein a metal soap drier is absorbed by a coating component such as a carbon black pigment, an organic red pigment, or a combination thereof. A feeder drier dissolves over time into the coating, thereby providing a continual supply of drier. An example a feeder drier include a tradename composition such as Troykyd® Perma Dry (Troy Corporation).

(2) Acids

An acid catalyzes amino resin cross-linking between a plurality of amino resins and/or an amino resin and an additional resin, though an acid is more effective in promoting cross-linking between the additional resin and an amino resin. A coating may comprise a strong acid, a weak acid, or a combination thereof. Examples of an acid include a strong acid or a weak acid. The rate of curing is typically accelerated by selection of a strong acid over a weak acid. Examples of a strong acid include, p-toluenesulfonic acid (“PTSA”), dodecylbenzenesulfonic acid (“DDBSA”), or a combination thereof. Examples of a weak acid include phenyl acid phosphate (“PAP”), butyl acid phosphate (“BAP”), or a combination thereof.

(3) Bases

A base catalyzes cross-linking between an acrylic resin and an epoxy resin in film formation. In specific aspects, the base comprises, for example, a dodecyl trimethyl ammonium chloride, a tri(dimethylaminomethyl)phenol, a melamine-formaldehyde resin, or a combination thereof.

(iv) Urethane Catalysts

In specific aspects, a urethane coating comprises a catalyst to accelerate the reaction between an isocyanate moiety and a reactive hydrogen moiety. Examples of such a urethane catalyst include a tin compound, a zinc compound, a tertiary amine, or a combination thereof. Examples of a zinc compound include zinc octoate, zinc naphthenate, or a combination thereof. Examples of a tin compound include dibutyltin dilaurate, stannous octoate, or a combination thereof. An example of a tertiary amine includes a triethylene diamine.

g. Antiskinning Agent

An antiskinning agent is a composition, other than a drier, that reduces film-formation at the coating-external environment interface, reduce shrinkage (“wrinkling”), or a combination thereof. Such antiskinning agents are often used to protect coatings from undesired film-formation after a container of coating has been opened, during normal film-formation, or a combination thereof. Examples of antiskinning agents, with commonly used coating concentrations in parentheses, include butyraloxime (0.2%), cyclohexanone oxime, dipentene, exkin 1, exkin 2, exkin 3, guaiacol (0.001% to 0.1%), methyl ethyl ketoxime (0.2%), pine oil (1% to 2%), or a combination thereof. Generally, an antiskinning agent acts by reducing the rate of film-formation and/or promotes even film-formation throughout a coating by slowing an oxidative reaction that occurs as part of film formation. Examples of antioxidant antiskinning agents include a phenolic antioxidant, an oxime, or a combination thereof. Example of a phenolic antioxidant includes guaiacol, 4-tert-butylphenol, or a combination thereof. Oximes tend to evaporate such as during film formation, are colorless, do not affect a coating's color property, and generally do not significantly alter the time of film-formation. Examples of an oxime include butyraldoxime, methyl ethyl ketoxime, cyclohexanone oxime, or a combination thereof. In certain facets, an oxime is used to slow skinning promoted by a copper drier.

h. Light Stabilizers

A coating, a film and/or a surface may be undesirably altered by contact with an environmental agent such as, for example, oxygen, pollution, water (e.g., moisture), and/or irradiation with light (e.g., UV light). To reduce such damaging alterations to a coating and/or film, it is contemplated that a coating composition may comprise a light stabilizer. A light stabilizer (“stabilizer”) is a composition that reduces or prevents damage to a coating, film and/or surface by an environmental agent. Such agents may alter the color, cause a separation between two layers of film (“delamination”), promote chalking, promote crack formation, reduce gloss, or a combination thereof. This is a particular problem for a film in an exterior environment, such as, for example, an automotive film. Additionally, wood surfaces are susceptible to damage by environmental agents, particularly UV light.

Typically, a light stabilizer may comprise a UV absorber, a radical scavenger, or a combination thereof. A UV absorber is a composition that absorbs UV light. Examples of UV absorbers include a hydroxybenzophenone, a hydroxyphenylbenzotriazole, a hydrozyphenyl-S-triazine, an oxalic anilide, yellow iron oxide, or a combination thereof. A hydroxyphenylbenzotriazole generally demonstrates the broadest range of UV wavelength absorption, and converts the absorbed UV light into heat. Additionally, a hydroxyphenylbenzotriazole and/or a hydrozyphenyl-S-triazine usually have the longest effective use in a film due to a higher resistance to photochemical reactions, relative to a hydroxybenzophenone or an oxalic anilide.

A radical scavenger light stabilizer (e.g., a sterically hindered amine) is a composition that chemically reacts with a radical (“free radical”). Examples of a sterically hindered amine (“hindered amine light stabilizer,” “HALS”) include the ester derivatives of decanedioic acid, such as HALS I [“bis(1,2,2,6,6,-pentamethyl-4-poperidinyl)ester”], which is used in non-acid catalyzed coatings; HALS II [“bis(2,2,6,6,-tetramethyl-1-isooctyloxy-4-piperidinyl)ester”], which is typically used in an acid catalyzed coating.

For embodiments wherein a coating, film, and/or surface is primarily located in-doors, a range of 1% to 3%, including all intermediate ranges and combinations thereof, of a light stabilizer relative to binder content is contemplated. A range of 1% to 5%, including all intermediate ranges and combinations thereof, of a light stabilizer relative to binder content is contemplated for exterior uses. Additionally, a combination of a UV absorber and a radical scavenger light stabilizer are contemplated in some embodiments, as the heat released by a UV absorber may promote radical formation. Light stabilizers are often commercially produced, and examples of UV absorber and/or a radical scavenger light stabilizer sold under a tradename include Tinuvin® (Ciba Specialty Chemicals) or Sanduvor® [Clariant LSM (America) Inc.].

i. Corrosion Inhibitors

A coating comprising a liquid component that comprises water, particularly a water-borne coating, may promote corrosion in a container comprising iron, particularly at the lining, seams, handle, etc. A corrosion inhibitor reduces corrosion by water or another chemical. Examples of a corrosion inhibitor includes a chromate, a phosphate, a molybdate, a wollastonite, a calcium ion-exchanged silica gel, a zinc compound, a borosilicate, a phosphosilicate, a hydrotalcite, or a combination thereof.

In certain embodiments, a corrosion inhibitor is an in-can corrosion inhibitor, a flash corrosion inhibitor, or a combination thereof. An in-can corrosion inhibitor (“can-corrosion inhibitor”) is a composition that reduces or prevents such corrosion. Examples of an in-can corrosion inhibitor are sodium nitrate, sodium benzoate, or a combination thereof. These compounds are typically used at a concentration of 1% each in a coating composition. In-can corrosion inhibitor are often commercially produced, and an example includes SER-AD® FA179 (Condea Servo LLC.), typically used at 0.3% in a coating composition. A flash corrosion inhibitor (“flash rust inhibitor”) is a composition that reduces or prevents corrosion produced by application of a coating comprising water to a metal surface (e.g., an iron surface). Often, in-can corrosion inhibitors at increased concentrations are added to a coating composition to act as a flash corrosion inhibitor. An example of a flash corrosion inhibitor includes sodium nitrite, ammonium benzoate, 2-amino-2-methyl-propan-1-ol (“AMP”), SER-AD® FA179 (Condea Servo LLC.), or a combination thereof. Standard procedures to determining the effectiveness of corrosion inhibition by a coating comprising a flash rust inhibitor are described, for example, in “ASTM Book of Standards, Volume 06.02, Paint—Products and Applications; Protective Coatings; Pipeline Coatings,” D5367-00, 2002.

j. Dehydrators

In some embodiments, preventing moisture from contacting coating component such as a binder, solvent, pigment, or a combination thereof, may be desired. For example, certain urethane coatings undergo film-formation in the presence of moisture, as well as produce a film with increased yellowing, increased hazing and/or decreased gloss. A dehydrator may be added during coating production and/or storage to reduce contact with moisture. Examples of a dehydrator include Additive TI (Bayer Corporation), Additive OF (Bayer Corporation), or a combination thereof. Additive TI comprises a compound with one reactive isocyanate moiety, and it is capable of reacting with compounds with a chemically reactive hydrogen such as water, an alcohol, a phenol, or an amide. However, in a reaction with water, the reaction products typically are carbon dioxide and toluenesulfonamide. The toluenesulfonamide is generally inert relative to a urethane binder, and soluble in many non-aqueous liquid components. In certain embodiments, a urethane coating may comprise 0.5% to 4% Additive TI. Additive OF is a dehydrator generally used in a urethane coating. In certain embodiments, a urethane coating may comprise 1% to 3% Additive OF.

k. Electrical Additives

In some embodiments, it is desirable to include an additive to alter an electrical property of a coating (e.g., electrical conductivity, electrical resistance). Examples of an additive to alter an electrical property of a coating and/or coating component include an anti-static additive, an electrical resistance additive, or a combination thereof. An anti-static additive may be included in a coating composition comprising a flammable component to reduce the chance of an electrostatic spark occurring and igniting the coating. An anti-static additive is a composition that increases the electrical conductivity of a coating. An example of a flammable component is a hydrocarbon solvent. Examples of an anti-static additive include Stadis® 425 (Octel-Starreon LLC USA), Stadis® 450 (Octel-Starreon LLC USA), or a combination thereof. An electrical resistance additive is a composition that reduces the resistance to electricity by a coating. An electrical resistance additive may be included in a coating to improve the ability of a coating to be applied to a surface using an electrostatic spray applicator. For example, an oxygenated compound (e.g., a glycol ether) often possesses a high electrical conductivity, which can make use of an electrostatic spray applicator to apply a coating comprising an oxygenated compound relatively more difficult than a similar coating lacking an oxygenated compound. Examples of an electrical resistance additive include Ramsprep, Byk-ES 80 (BYK-Chemie GmbH), or a combination thereof. Byk-ES 80 comprises, for example, an unsaturated acidic carboxylic acid ester alkylolammonium salt, and typically is added between 0.2% and 2% to a coating composition. Additionally, techniques in the art for determining an electrical property (e.g., electrical resistance) of a coating comprising an electrical additive may be used (see, for example, “ASTM Book of Standards, Volume 06.01, Paint—Tests for Chemical, Physical, and Optical Properties; Appearance,” D5682-95, 2002).

1. Anti-Insect Additives

Certain coatings may serve a protective role for a surface or surrounding environment against insects, and thus may comprise an anti-insect agent. An example of a surface where a coating comprising an anti-insect agent may be desirable is a wooden surface. Examples of an area where coating comprising an anti-insect agent may be desirable would be a storage facility, such as a cargo hold of a ship or railcar. An anti-insect agent is a composition that, upon contact, is detrimental to the well-being (e.g., life, reproduction) of an invertebrate pest (e.g., an insect, an arachnid, etc). Examples of anti-insect additives that have been used in coatings include copper naphthenate, tributyl tin oxide, zinc oxide, 6-chloro epoxy hydroxy naphthalene, 1-dichloro 2,2′bis-(p-chlorophenyl)ethane, or a combination thereof.

5. Coating Preparation

A coating may comprise insoluble particulate material. Particulate material may comprise a primary particle, an agglomerate, an aggregate, or a combination thereof. A primary particle is a single particle not in contact with a second particle. An agglomerate is two or more particles in contact with each other, and generally can be separated by a dispersion technique, a wetting agent, a dispersant, or a combination thereof. An aggregate is two or more particles in contact with each other, which are generally difficult to separate by a dispersion technique, a wetting agent, a dispersant, or a combination thereof.

Usually, a pigment, an extender, certain types of rheology modifiers, certain types of dispersants, or a combination thereof are the major sources of particulate material in a coating. A cell-based particulate material will also be a source of particulate material in a coating. In certain embodiments, a cell-based particulate matter may be used in combination with and/or as a substitute for a pigment, an extender, a rheology modifier, a dispersant, or a combination thereof. In specific facets, a cell-based particulate matter may substitute for 0.000001% to 100%, including all intermediate ranges and combinations thereof, of a pigment, an extender, a rheology modifier, a dispersant, or a combination thereof. In certain embodiments, it is contemplated that a coating or other surface treatment wherein the cell-based particulate material tends to be at or near the coating/surface treatment-external environment interface. Preparation of such a coating or surface treatment wherein a particulate material is at or near the coating/surface treatment-external environment interface may be accomplished by formulation to enhance the ballooning, blooming, floating, flooding, etc. of the particulate material. It is contemplated that any technique used in the preparation of a coating that comprises a pigment, extender or any other form of particulate material described herein or in the art may be used in the preparation of a coating comprising the cell-based particulate material. Incorporation of particulate materials (e.g., pigments), assays for determining a rheological property and/or a related property (e.g., viscosity, flow, molecular weight, component concentration, particle size, particle shape, particle surface area, particle spread, dispersion, flocculation, solubility, oil absorption values, CPVC, hiding power, corrosion resistance, wet abrasion resistance, stain resistance, optical properties, porosity, surface tension, volatility, settling, leveling, sagging, slumping, draining, floating, flooding, cratering, foaming, splattering) of a coating component and/or a coating (e.g., pigment, binder, vehicle, surfactant, dispersant, paint) and procedures for determining such properties, as well as procedures for large scale (e.g., industrial) coating preparation (e.g., wetting, pigment dispersion into a vehicle, milling, letdown) are described in, for example, in Patton, T. C. “Paint Flow and Pigment Dispersion, A Rheological Approach to Coating and Ink Technology,” 1979.

In many embodiments, dispersion of the particulate material is promoted by application of physical force (e.g., impact, shear) to the composition. Techniques such as grinding and/or milling are typically used to apply physical force for dispersion of particulate matter. Though it is contemplated that such application of physical force may be used in the dispersal of the cell-based particulate material, such force may damage the structural integrity of the cell wall and/or cell membrane that confers size and shape to the material. The average particle size and shape will be altered by the degree of damage to the cell wall and/or cell membrane, which may alter a physical property, a chemical property, an optical property, or a combination thereof, of a cell-based particulate material. Examples of a physical property that may be altered by cell fragmentation include a rheological property, such as the contribution to viscosity, flow, etc., the tendency to form a primary particle, an agglomerate, an aggregate, etc. An example of a chemical property that may be altered includes allowing greater contact between amine and hydroxyl moieties of internally located biomolecules (e.g., a proteinaceous molecule) with a coating component, which may undergo a chemical reaction (e.g., crosslinking) with a binder. An example of an optical property that may be altered includes an alteration in the gloss characteristic of a coating and/or film by a reduction in particle size due to cell fragmentation.

For example, during typical preparation of a water-borne and/or solvent-borne coating comprising particulate material such as a pigment and/or extender, the particulate material is dispersed into a paste known as a “grind” or “millbase.” A combination of a binder and a liquid component know as a “vehicle” is used to disperse the particulate material into the grind. Often, a wetting additive is included to promote dispersion of the particulate material. Additional vehicle and/or additives are admixed with the grind in a stage referred to as the “letdown” to produce a coating of a desired composition and/or properties. These techniques and others for coating preparation in the art include, for example, in “ASTM Book of Standards, Volume 06.01, Paint—Tests for Chemical, Physical, and Optical Properties; Appearance,” D6619-00, 2002; in “Paint and Surface Coatings, Theory and Practice, Second Edition,” (Lambourne, R. and Strivens, T. A., Eds.), pp. 286-329, 1999; and in “Paints, Coatings and Solvents, Second, Completely Revised Edition,” (Stoye, D. and Freitag, W., Eds.) pp. 178-193, 1998. It is specifically contemplated that these techniques may be used in preparing a coating comprising the cell-based particulate matter, wherein the particulate matter is treated as a pigment, extender, or other such particulate material dispersed into a coating.

In another example, the effectiveness of the convertion of agglomerates and/or an aggregates into primary particles in the grind (e.g., pigments, pigment-vehicle combinations, pastes), and latter stages (e.g., lacquer, paint) are typically measured to insure quality, using techniques such as, for example, those described in “ASTM Book of Standards, Volume 06.01, Paint—Tests for Chemical, Physical, and Optical Properties; Appearance,” D1210-96, 2002; “ASTM Book of Standards, Volume 06.02, Paint—Products and Applications; Protective Coatings; Pipeline Coatings,” D2338-02, D1316-93, and D2067-97, 2002; and in “ASTM Book of Standards, Volume 06.03, Paint—Pigments, Drying Oils, Polymers, Resins, Naval Stores, Cellulosic Esters, and Ink Vehicles,” D185-84, 2002. It is specifically contemplated that these techniques for the preparation of coatings comprising a pigment, exender, or other particulate material may be used in the preparation of a coating comprising a cell-based particulate material.

In a further example, a cell-based particulate material may be adapted for use in standard coating formulation techniques to improve a coating composition for desired properties. The pigment volume concentration is the volume of pigment in the total volume solids of a dry film. The volume solids is the fractional volume of binder and pigment in the total volume of a coating. It is contemplated that in calculating the PVC, the content of a cell-based particulate material would be included in this or related calculations as a pigment or extender. A related calculation to the PVC that is specifically contemplated is the critical pigment volume concentration (“CPVC”) is the formulation of pigment and binder wherein the coating comprises the minimum amount of binder to fill the voids between the pigment particles. A pigment to binder concentration that exceeds the CVPC threshold produces a coating with empty spaces wherein gas (e.g., air, evaporated liquid component), may be trapped. Various properties rapidly change above the CPVC. For example, corrosion resistance, abrasion (e.g., scrub resistance), stain resistance, opacity, moisture resisitance, rigidity, gloss, or a combination thereof, are more rapidly reduced above the CPVC, while reflectance is often increased. However, in certain embodiments, coating may be formulated above the CPVC and still preduce a film suitable for given use upon a surface. Standard procedures for determining CPVC in the art may be used [see, for example, in “ASTM Book of Standards, Volume 06.01, Paint—Tests for Chemical, Physical, and Optical Properties; Appearance,” D1483-95, D281-95, and D6336-98, 2002; and in “Paint and Coating Testing Manual, Fourteenth Edition of the Gardner-Sward Handbook,” (Koleske, J. V. Ed.), pp. 252-258, 1995].

The physical and/or optical properties of a coating are affected by the size of particulate material comprised within the coating. For example, inclusion of a physically hard particulate material, such as a silica extender, may increase the abrasion resistance of a film. In another example, gloss is reduced when particulate material of a larger average particle size increases the roughness of the surface of a coating and/or film. Standard procedures for determining particle properties (e.g., size, shape) in the art may be used (see, for example, “ASTM Book of Standards, Volume 06.03, Paint—Pigments, Drying Oils, Polymers, Resins, Naval Stores, Cellulosic Esters, and Ink Vehicles,” D1366-86 and D3360-96, 2002; and in “Paint and Coating Testing Manual, Fourteenth Edition of the Gardner-Sward Handbook,” (Koleske, J. V. Ed.), pp. 305-332, 1995).

It is also contemplated that a biomolecular composition, particularly one prepared as a particulate or powder material, may be incorporated into a powder coating. Specific procedures for determining the properties (e.g., particle size, surface coverage, optical properties) of a powder coating and/or film have been described, for example, in “ASTM Book of Standards, Volume 06.02, Paint—Products and Applications; Protective Coatings; Pipeline Coatings,” D3451-01, D2967-02a, D4242-02, D5382-02 and D5861-95, 2002.

In some embodiments, the dispersion of particulate material (“fineness of grind”) in a coating is, in Hegman units (“Hu”), 0.0 Hu to 8.0 Hu, including all intermediate ranges and combinations thereof. The dispersion of particulate material content of a coating can be empirically determined, for example, as described in “ASTM Book of Standards, Volume 06.01, Paint—Tests for Chemical, Physical, and Optical Properties; Appearance,” D1210-96, 2002. The size of particulate matter in a coating can affect gloss, with smaller particle size generally more conducive for a higher gloss property of a coating and/or film. It is contemplated that a whole cell particulate material will possess similar size and shape as the organism from which it was derived. For example, E. coli is about 2 μm in length and 0.8 μm in diameter, maize cells vary more in size, but a size of about 65 μm in diameter may be found in some cell types, and Saccaromyces cerivsia is about 10 μm in diameter. Of course, processing and purifying techniques may reduce the particle size by fragmentation of the cell wall and membrane, and it is contemplated that a biomolecular composition may be prepared to an average particle size for a specific purpose (e.g., gloss). In certain facets, a visibly coarse and/or low gloss coating (e.g., a low gloss finish, a flat latex paint) has a dispersion of particulate material of 2.0 Hu to 4.0 Hu. A particle size of 100 μm to 50 μm is associated with a dispersion of 0.0 Hu to 4.0 Hu. In some aspects, a semi-gloss or gloss coating has a dispersion of particulate material of 5.0 Hu to 7.5 Hu. A particle size of 50 μm to 40 μm, 40 μm to 26 μm, 26 μm to 13 μm, and 13 μm to 6 μm is associated with a dispersion of 4.0 Hu to 5.0 Hu, 5.0 Hu to 6.0 Hu, 6.0 Hu to 7.0 Hu, and 7.0 Hu to 7.5 Hu, respectively. In other aspects, a high gloss coating has a dispersion of particulate material of 7.5 Hu to 8.0 Hu. A particle size of 6 μm to 3 μm and 3 μm to 0.1 μm is associated with a dispersion of 7.5 Hu to 7.75 Hu and 7.75 Hu to 8.0 Hu, respectively. In embodiments wherein a coating comprises a combination of particulate materials, wherein the different particulate materials such as a combination of a cell-based particulate material and one or more of different pigments, with each type of particulate material possessing a different average particle size, it is contemplated that the gloss will be affected by the particle size of the largest type of particulate material added. However, gloss can also be empirically determined for a coating and/or film, as described herein or by techniques in the art in light of the present disclosures.

6. Empirically Determining the Properties of Biomolecule, Coatings and/or Film

A coating with a desired set of properties for a particular use may be prepared by varying the ranges and/or combinations of coating components, and such coating selection and preparation may be done in light of the present disclosures. For example, a variety of assays are available to measure various properties of a coating, coating application, and/or a film to determine the degree of suitability of a coating composition for use in a particular use (see, for example, in “Hess's Paint Film Defects: Their Causes and Cure,” 1979).

It is contemplated that in general embodiments, a coating comprising a biomolecular composition may be subjected to one or more of such assays. Additionally, a biomolecular composition may further comprise a desired biomolecule (e.g., a colorant, an enzyme), whether endogenously or recombinantly produced, that may confer a desired property to a coating and/or film. As used herein, “bioactivity” refers to desired property such as color, enzymatic activity, etc, conferred to a coating by a biomolecular composition. As used herein, “bioactivity resistance” refers to the ability of a biomolecular composition to confer a desired property during and/or after contact with a stress condition normally assayed for in a standard coating and/or film assay procedure. Examples of such a stress condition includes, for example, a temperature (e.g., a baking condition), contact with a coating component (e.g., an organic liquid component), contact with a chemical reaction (e.g., thermosetting film formation), contact with coating and/or film damaging agent (e.g., weathering, detergents, solvents), etc. In specific facets, wherein a biomolecular composition comprises a desired biomolecule, a biomolecule may possess a greater bioactivity resistance such as determined with standard assay procedure.

It is contemplated that such bioactivity resistance may be determined using a standard procedure for a coating and/or film described herein or in the art, in light of the present disclosures. In an example, any assay described herein or in the art in light of the present disclosures may be used to determine the bioactivity resistance wherein an enzyme retains detectable enzymatic activity upon contact with a condition typically encountered in a standard assay. Additionally, in certain aspects, it is contemplated that a coating and/or film comprising an enzyme may lose part of all of a detectable, desirable bioactivity during the period of time of contact with standard assay condition, but regain part or all of the enzymatic bioactivity after return to non-assay conditions. An example of this process is the thermal denaturation of an enzyme at an elevated temperature range into a configuration with lowered or absent bioactivity, followed by refolding of an enzyme, upon return to a more suitable temperature range for the enzyme, into a configuration possessing part or all of the enzymatic bioactivity detectable prior to contact with the elevated temperature. In another example, an enzyme may demonstrate such an increase in bioactivity upon removal of a solvent, chemical, etc.

In some embodiments, an enzyme identified as having a desirable enzymatic property for one or more target substrates may be selected for incorporation into a composition. The determination of an enzymatic property may be conducted using any technique described herein or in the art, in light of the present disclosures. For example, the determination of the rate of cleavage of a substrate, with or without a competitive or non-competitive enzyme inhibitor, can be utilized in determining the enzymatic properties of an enzyme, such as Vₘₐₓ, Kₘ, K_{cat}/Kₘ and the like, using analytical techniques such as Lineweaver-Burke analysis, Bronsted plots, etc Brockerhoff, Hans and Jensen, Robert G. “Lipolytic Enzymes”, pp 10-24, 1974; Dumas, D. P. et al., 1989a; Dumas, D. P. et al., 1989b; Dumas, D. P. et al., 1990; Caldwell, S. R. and Raushel, F. M., 1991c; Donarski, W. J. et al., 1989; Raveh, L. et al., 1992; Shim, H. et al., 1998; Watkins, L. M. et al., 1997a; diSioudi, B. et al., 1999; Hill, C. M., 2000; Hartleib, J. and Ruterjans, H., 2001b; Lineweaver, H. and Burke, D., 1934; Segel, I. H., 1975). It is contemplated that any such analysis may be used to identify an enzyme with a specifically desirable enzymatic property for one or more substrates.

For example, lipolytic enzymes and phosphoric triester hydrolases have demonstrated the ability to degrade a wide variety of lipids and OP compounds, respectively. Methods for measuring the ability of an enzyme to degrade a lipid or an OP compound are described herein as well as are in the art. It is contemplated that any such technique may be utilized to determine enzymatic activity of a composition for a particular lipid or OP compound.

For example, techniques for measuring the enzymatic degradation, specificity (e.g., positional specificity) for various lipids comprising an ester or other hydrolysable moiety, including a triglyceride such as triolein, olive oil, or tributyrin; chromogenic substrates such as 4-methylumbelliferone, or 4-methylumbelliferone; or radioactively labeled glycerol ester substrates, such as glycerol [³H]oleic acid esters; are known to those of skill in the art (see, for example, Brockerhoff, Hans and Jensen, Robert G. “Lipolytic Enzymes.” pp-25-34, 1974). To measure lipolytic enzyme activity against substrates, molecular monolayers of lipid substrates may be used to control variables such as pressure, charge potential, density, interfacial characteristics, enzyme binding, the effects of an inhibitor, in measuring lipolytic enzyme kinetics [see for example, Gargouri, Y. et al., 1989; Melo, E. P. et al., 1995; In “Methods and Molecular Biology, Volume 109 Lipase and Phospholipase Protocols.” (Mark Doolittle and Karen Reue, Eds.), pp 279-302, 1999].

Measuring the activity, stability, and other properties of lipolytic enzymes are known to those of skill in the art. For example, methods for measuring the activity of phospholipase A₂ and phospholipase C by the thin layer chromatography product separation, the fluorescence change of labeled substrates (e.g., dansyl-labeled glycerol, pyrene-PI, pyrene-PG), the release of products from radiolabled substrates (e.g., [³H]Plasmenylcholine) have been described [see for example, in “Methods and Molecular Biology, Volume 109 Lipase and Phospholipase Protocols.” (Mark Doolittle and Karen Reue, Eds.), pp. 1-17, 31-48, 1999]. Similarly, the release of fluorogenic products from substrates such as, for example, 1-trinitrophenyl-aminododecanoyl-2-pyrenedecanoyl-3-O-hexadecyl-sn-glycerol, or radioactive products from radiolabled substrates such as, for example, [³H]triolein; glycerol tri[9, 10(n)-[³H]oleate; cholesterol-[1-¹⁴C]-oleate; 1(3)-mono-[³H]oleoyl-2-O-mono-oleyleglycerol (a.k.a. [³H]-MOME) and 1(3)-mono-oleoyl-2-O-mono-oleylglycerol (a.k.a. MOME); by lipolytic enzymes that catalyze hydrolysis of tri, di, or monoacylglycerols and sterol esters may be used to measure such enzymes' activity [see for example, in “Methods and Molecular Biology, Volume 109 Lipase and Phospholipase Protocols.” (Mark Doolittle and Karen Reue, Eds.), pp. 18-30, 59-121, 1999]. Other assays using radiolabeled E. coli membranes to measure phospholipase activity in comparison to photometric and other assays has also been described [In “Esterases, Lipases, and Phospholipases from Structure to Clinical Significance.” (Mackness, M. I. and Clerc, M., Eds.), pp 263-272, 1994].

It is contemplated that one of skill in the art may readily modify these types of techniques by replacement of a purified or immobilized enzyme typically assayed with compositions such as, for example, a biomolecular composition, a coating, a surface treatment, to assay and characterize the enzymatic activity of such a composition. Such measurements of the enzymatic activity of compositions may be used to select formulations with the desired activity properties of stability, activity, and such like, in different environmental conditions (e.g., pressure, interfacial characteristics, the effects of an inhibitor, temperature, detergent, organic solvent, etc.) or after contact with different substrates (e.g., contact with substrates mimicking vegetable oil properties vs. those for a sterol) to assess properties such as the substrate preference, enantiomeric specificity, kinetic properties, etc. of a composition.

Techniques for measuring the kinetics of enzymatic degradation for various OP-compounds comprising a P—S bond at the phosphorous center (e.g., an OP-phosphonothiolate) such as VX [“EA 1701,” “TX60,” “O-ethyl-S-(diisopropylaminoethyl)methylphosphonothioate”]; Russian VX [“R-VX,” “O-isobutyl-S-(diisopropylaminoethyl)methylphosphonothioate”], tetriso [“O,O-diisopropyl S-(2-diisoprpylaminoethyl)phosphorothiolate”], echothiophate (“phospholine,” “O,O-diethyl-phosphorothiocholine”), malathion [“phosphothion,” “S-(1,2-dicarbethoxyethyl)-O,O-dimethyl dithiophosphate”], dimethoate [“Cygon®,” “Dimetate®,” “O,O-dimethyl-S-(N-methylcarbomoylmethyl)phosphorodithioate”], EA 5533 [“OSDMP,” “O,S-diethyl methylphosphonothioate”], IBP (“Kitazin P,” “O,O-diisopropyl-5-benzylphosphothioate”), acephate (“O,S-dimethyl acetyl phosphoroamidothioate”), azinophos-ethyl [“S-(3,4-dihydro-4-oxobenzo(d)-1,2,3-triazin-3-yl methyl-O,O-diethyl)phosphorothioate”], demeton S [“VX analogue,” “O,O-diethyl-S-2-ethylthio]ethyl phosphorothioate”], malathion [“Phosphothion,” “S-(1,2-dicarbethoxyethyl)-O,O-dimethyl dithiophosphate”], and phosalone [“O,O-diethyl-S-(6-chloro-2-oxobenzoxazolin-3-yl-methyl)phosphorodithioate”], are known to those of skill in the art (see, for example, diSioudi, B. D. et al., 1999; Hoskin, F. C. G. et al., 1995; Watkins, L. M. et al., 1997a; Kolakowski, J. E. et al., 1997; Gopal, S. et al., 2000; and Rastogi, V. K. et al., 1997).

Techniques for measuring the kinetics of enzymatic detoxification for various OP-compounds comprising a P—F bond at the phosphorous center (e.g., an OP-phosphonofluoridate) such as soman (“1,2,2-trimethylpropyl-methylphosphonofluoridate”), sarin (“isopropylmethylphosphonofluoridate”), DFP (“O,O-diisopropyl phosphorofluoridate”), alpha (“1-ethylpropylmethylphosphonofluoridate”), and mipafox (“N,N′-diisopropylphosphorofluorodiamidate”) have been described (see, for example Dumas, D. P. et al., 1990; Li, W.-S. et al., 2001; diSioudi, B. D. et al., 1999; Hoskin, F. C. G. et al., 1995; Gopal, S. et al., 2000; and DeFrank, J. and Cheng, T., 1991).

A technique for measuring the kinetics of enzymatic detoxification for an OP-compound comprising a P—CN bond at the phosphorous center (e.g., an OP-phosphonocyanate) such as tabun (“ethyl N,N-demethylamidophosphorocyanidate”) has been described (see, for example, Raveh, L. et al., 1992).

Techniques for measuring the kinetics of enzymatic detoxification for various OP-compounds comprising a P-0 bond at the phosphorous center (e.g., an OP-triester) such as paraoxon (“diethyl p-nitrophenylphosphate”), the soman analogue O-pinacolyl p-nitrophenyl methylphosphonate, the sarin analogue O-isopropyl p-nitrophenyl methylphosphonate, NPPMP (“p-nitrophenyl-o-pinacolyl methylphosphonate”), coumaphos [“O,O-diethyl O-(3-chloro-4-methyl-2-oxo-2H-lbenzyran-7-yl)phosphorothioate], cyanophos [“O,O-dimethyl p-cyanophenyl phosphorothioate”], diazinon (“O,O-diethyl 0-2-iso-propyl-4-methyl-6-pyrimidyl phosphorothiate”), dursban (“O,O-diethyl 0-3,5,6-trichloro-2-pyridyl phosphorothioate”), fensulfothion {“O,O-diethyl [p-(methylsulfinyl)phenyl] phosphorothioate”}, parathion (“O,O-diethyl O-p-nitrophenyl phosphorothioate”), methyl parathion (“O,O-dimethyl p-nitrophenyl phosphorothioate”), ethyl parathion [“O,O-diethyl-O-(4-nitrophenyl)phosphorothioate”], EPN (“O-ethyl O-(4-nitrophenyl)phenylphosphonothioate”), DEPP (“diethylphenylphosphate”), NPEPP (“p-nitrophenylethylphenylphosphinate”) have been described (see, for example, Dumas, D. P. et al., 1990; Li, W.-S. et al., 2001; diSioudi, B. D. et al., 1999; Watkins, L. M. et al., 1997a; Gopal, S. et al., 2000; Mulbry, W. and Karns, J., 1989; Hong, S.-B. and Raushel, F. M., 1996; and Dumas, D. P. et al., 1989b).

In one example, the cleavage rate of a phosphonothiolate OP substrate comprising a P—S bond can be measured using a method known as the Ellman reaction. All such substrates produce a P—S bond cleavage product comprising a free thiol group, which can chemically react with the Ellman's reagent, 5,5′-dithio-bis-2-nitrobenzoic acid (“DTNB”). This reaction produces 5′-thiol-2-nitrobenzoate anions with a maximum absorbency at 412 nm. P—S cleavage can be determined by the appearance of the free thiol group, measured using a spectrophotometer (Rastogi, V. H. et al., 1997; Gopal, S. et al., 2000; diSioudi, B. et al., 1999; Watkins, L. M. et al., 1997a; Hoskin, F. C. G. et al., 1995; Chae, M. Y. et al., 1994; Ellman, G. L. et al., 1961).

In an additional example, the cleavage of an OP substrate can be measured by detecting the production of a cleavage product that comprises a released ion. In a further example, the cleavage of a phosphonofluoridate can be measured by the release of cleavage product comprising a fluoride ion (F⁻) using a fluoride ion specific electrode and a pH/mV meter (Hartleib, J. and Ruterjans, H., 2001a; Gopal, S. et al., 2000; diSioudi, B. et al., 1999; Watkins, L. M. et al., 1997a; DeFrank, J. and Cheng, T., 1991; Dumas, D. P. et al., 1990; Dumas, D. P. et al., 1989a). In another example, the cleavage of a phosphonocyanate can be measured by the release of a cleavage product comprising a cyanide ion (CN⁻) using a cyanide selective electrode with a pH meter (Raveh, L. et al., 1992).

In another example, cleavage of an OP substrate can be measured, for example, by ³¹P NMR spectroscopy. For example, the disappearance of VX and the formation of the cleavage product ethyl methylphosphonic acid (“EMPA”), has been measured using this technique (Kolakowski, J. E. et al., 1997; Lai, K. et al., 1995). In another example, the disappearance of tabun and the appearance of the N,N-dimethylamindophosphosphoric acid cleavage product has been measured by ³¹P NMR spectroscopy (Raveh, L. et al., 1992). In a further example, the disappearance of DFP and appearance of a F— cleavage product has been determined using ¹⁹F⁻ and ³¹P NMR spectroscopy (Dumas, D. P. et al., 1989a).

The cleavage of many OP compounds' such as paraoxon, coumaphos, cyanophos, diazinon, dursban, fensulfothion, parathion, methyl parathion, DEPP, and various phosphodiesters, can be determined by measuring the production of a cleavage product spectrophotometrically at visible or UV wavelengths (Dumas, D. P. et al., 1989b). For example, the cleavage of DEPP can be measured at 280 nm, using a spectrophotometer to detect a phenol cleavage product (Watkins, L. M. et al., 1997a; Hong, S.-B. and Raushel, F. M., 1996). In a further example, various phosphodiesters (e.g., ethyl-4-nitrophenyl phosphate) have been made to evaluate OPH cleavage rates, and their cleavage measured at 280 nm by the production of a substituted phenol cleavage product (Shim, H. et al., 1998). In a further example, paraoxon is often used as to measure OPH activity, because it is both rapidly hydrolyzed by the enzyme and produces a visible cleavage product. To determine kinetic properties, the production of paraoxon's cleavage product, p-nitrophenol, is measured with a spectrophotometer at 400 nm or 420 nm (Dumas, D. P. et al., 1990; Kuo, J. M. and Raushel, F. M., 1994; Watkins, L. M. et al., 1997a; Gopal, S. et al., 2000). In an additional example, NPPMP cleavage can also be measured by the release of p-nitrophenol as a cleavage product (diSioudi, B. et al., 1999). In a further example, chiral and non-chiral phosphotriesters have been created to produce p-nitrophenol as a cleavage product, and thus adapt the method used in measuring paraoxon cleavage in determining the general binding and/or cleavage preference of an enzyme for a phosphoryl group Sₚ enantiomer, Rₚ enantiomer or non-chiral substrate (Chen-Goodspeed, M. et al., 2001a; Chen-Goodspeed, M. et al., 2001b; Wu, F. et al., 2000a; Steubaut, W. et al., 1975). In an example, chiral sarin and soman analogues have been created wherein the fluoride comprising moiety of the P—F bond has been replaced byp-nitrophenol, allowing detection of the CWA analogs' cleavage rates using the adapted method for paraoxon cleavage measurement (Li, W.-S. et al., 2001).

Other techniques are known to those of skill in the art for measuring OP detoxification activity, such as, for example, determining the loss of acetylcholinesterase inhibitory potency of an OP compound due to contact with an enzyme (Hoskin, F. C. G., 1990; Luo, C. et al., 1999; Ashani, Y. et al., 1998).

General procedures for empirically determining the purity/properties of various coating components and/or coating compositions in the art may be used. Such procedures include measurement of density, volume solids and/or specific gravity, of a coating component and/or coating composition, for purposes such as verification of component identity, aid in coating formulation, maintaining coating batch to batch consistency, etc. Examples of standard techniques for determining density of various solvents, liquids (e.g., a liquid coating), pigments, coatings (e.g., a powder coating) include those described in “ASTM Book of Standards, Volume 06.04, Paint—Solvents; Aromatic Hydrocarbons,” D2935-96, D1555M-00, D1555-95, and D3505-96, 2002; “ASTM Book of Standards, Volume 06.01, Paint—Tests for Chemical, Physical, and Optical Properties; Appearance,” D1475-98 and D215-91, 2002; “ASTM Book of Standards, Volume 06.03, Paint—Pigments, Drying Oils, Polymers, Resins, Naval Stores, Cellulosic Esters, and Ink Vehicles,” D153-84 and D153-84, 2002; “ASTM Book of Standards, Volume 06.02, Paint—Products and Applications; Protective Coatings; Pipeline Coatings,” D5965-02, 2002; and “Paint and Coating Testing Manual, Fourteenth Edition of the Gardner-Sward Handbook,” (Koleske, J. V. Ed.), pp. 289-304, 1995.

Standard surface specification and/or procedures for preparing a surface (e.g., glass, wood, steel) for empirically measuring a physical and/or visual property of a coating (e.g., a paint, a varnish, a lacquer) and/or film are have been described (see, for example, “ASTM Book of Standards, Volume 06.01, Paint—Tests for Chemical, Physical, and Optical Properties; Appearance,” D3891-96, D609-00, and D2201-99, 2002; and “ASTM Book of Standards, Volume 06.02, Paint—Products and Applications; Protective Coatings; Pipeline Coatings,” D358-98, D4227-99, and D4228-99, 2002). Specific procedures for preparing a metal surface and an evaluating a coating (e.g., a primer, a paint) applied to a metal surface from the art may be used (see, for example, “ASTM Book of Standards, Volume 06.02, Paint—Products and Applications; Protective Coatings; Pipeline Coatings,” D3276-00, D5161-96, D4417-93, D3322-82, D2092-95, D5065-01, D5723-95, D6386-99, and D6492-99, 2002). Specific procedures for evaluating a coating applied to a plastic surface from the art may be used (see, for example, “ASTM Book of Standards, Volume 06.02, Paint—Products and Applications; Protective Coatings; Pipeline Coatings,” D3002-02, 2002).

Standard procedures for determining the stability of a coating (e.g., a water-borne coating, a UV irradiation cured coating) in a container prior and/or after opening the container from the art may be used (see, for example, “ASTM Book of Standards, Volume 06.02, Paint—Products and Applications; Protective Coatings; Pipeline Coatings,” D2243-95 and D4144-94, 2002).

Standard procedures for evaluating an applicator (e.g., a brush, a roller, a fabric, a spray applicator, an electrocoat bath) and/or a coating being applied by an applicator may be used (see, for example, “ASTM Book of Standards, Volume 06.02, Paint—Products and Applications; Protective Coatings; Pipeline Coatings,” D6737-01, D5913-96, D5959-96, D5301-92, D5068-02, D5069-92, D4707-97, D5286-01, D6337-98, D4285-83, and D5327-97, 2002; and “ASTM Book of Standards, Volume 06.01, Paint—Tests for Chemical, Physical, and Optical Properties; Appearance,” D1978-91, D5794-95, D4370-01, D4399-90, and D4584-86, 2002.

Standard procedures for preparing a coating (e.g., a paint, a varnish, a lacquer) and/or film layer upon a surface for empirically measuring a physical and/or visual property may be used (see, for example, “ASTM Book of Standards, Volume 06.01, Paint—Tests for Chemical, Physical, and Optical Properties; Appearance,” D3924-80, D823-95, and D4708-99, 2002; “ASTM Book of Standards, Volume 06.02, Paint—Products and Applications; Protective Coatings; Pipeline Coatings,” D6206-97, D1734-93, and D4400-99, 2002; and “Paint and Coating Testing Manual, Fourteenth Edition of the Gardner-Sward Handbook,” (Koleske, J. V. Ed.), pp. 415-423, 1995.

Standard procedures for empirically determining the degree and duration of film formation of various coating compositions in the art may be used. Example of a standard technique for determining the degree/duration of film formation by loss of a volatile coating component and/or a cross-linking reaction for a coating (e.g., an oil-coating, a UV cured coating, an thermosetting powder coating) include those described in “ASTM Book of Standards, Volume 06.01, Paint—Tests for Chemical, Physical, and Optical Properties; Appearance,” D3539-87, D1640-95 and D5895-01e1, 2002; “ASTM Book of Standards, Volume 06.02, Paint—Products and Applications; Protective Coatings; Pipeline Coatings,” D4217-02, D3732-82, D2091-96, D711-89, D4752-98, and D5909-96a, 2002; “ASTM Book of Standards, Volume 06.03, Paint—Pigments, Drying Oils, Polymers, Resins, Naval Stores, Cellulosic Esters, and Ink Vehicles,” D2575-70 and D2354-98, 2002; and “Paint and Coating Testing Manual, Fourteenth Edition of the Gardner-Sward Handbook,” (Koleske, J. V. Ed.), pp. 407-414, 1995. Additionally, the temperature generated by a film formation reaction by a coating (e.g., a wood coating) may also be determined by techniques in the art (see, for example, “ASTM Book of Standards, Volume 06.02, Paint—Products and Applications; Protective Coatings; Pipeline Coatings,” D3259-95, 2002). Further, standard techniques for evaluating baking conditions on an organic coating and/or film may be used (see, for example, “ASTM Book of Standards, Volume 06.01, Paint—Tests for Chemical, Physical, and Optical Properties; Appearance,” D2454-95, 2002).

In embodiments wherein film formation at ambient conditions is used for a coating, a standard procedure in that art may be used for measuring film formation rate and/or stages (see for example, “ASTM Book of Standards, Volume 06.01, Paint—Tests for Chemical, Physical, and Optical Properties; Appearance,” D1640-95, 2002. In certain aspects wherein the ability of an oil to undergo film formation is to be determined, a standard procedure described in “ASTM Book of Standards, Volume 06.03, Paint—Pigments, Drying Oils, Polymers, Resins, Naval Stores, Cellulosic Esters, and Ink Vehicles,” D1955-85, 2002, may be used. In embodiments wherein the hardness of a film produced by a coating composition is measured (e.g., an organic coating), a standard procedure such as, for example, “ASTM Book of Standards, Volume 06.01, Paint—Tests for Chemical, Physical, and Optical Properties; Appearance,” D3363-00, D4366-95, and D1474-98, 2002.

Examples of a standard technique for determining the coating and/or film thickness after application to various surface types are described in “ASTM Book of Standards, Volume 06.01, Paint—Tests for Chemical, Physical, and Optical Properties; Appearance,” D1212-91, D4414-95, D1005-95, D1400-00, D1186-01, and D6132-97, 2002; “ASTM Book of Standards, Volume 06.02, Paint—Products and Applications; Protective Coatings; Pipeline Coatings,” D5235-97, D4138-94, D2200-95, and D5796-99, 2002; and “Paint and Coating Testing Manual, Fourteenth Edition of the Gardner-Sward Handbook,” (Koleske, J. V. Ed.), pp. 424-438, 1995.

Examples of a standard technique for determining the adhesion of a coating and/or film to various surface types are described in “ASTM Book of Standards, Volume 06.01, Paint—Tests for Chemical, Physical, and Optical Properties; Appearance,” D3359-02, D5179-98, and D2197-98, 2002; “ASTM Book of Standards, Volume 06.02, Paint—Products and Applications; Protective Coatings; Pipeline Coatings,” D4541-02 D3730-98, D4145-83, D4146-96, and D6677-01, 2002; and “Paint and Coating Testing Manual, Fourteenth Edition of the Gardner-Sward Handbook,” (Koleske, J. V. Ed.), pp. 513-524, 1995. Additionally, standard procedures for determining the ability of one or more layers of a multicoat system to function (e.g., adhere, weather) together are described in, for example, “ASTM Book of Standards, Volume 06.02, Paint—Products and Applications; Protective Coatings; Pipeline Coatings,” D5064-01, 2002.

Various standard techniques for determining the physical properties (e.g., flexibility, tensile strength, toughness, impact resistance, hardness, mar resistance, blocking resistance) relevant to the durability of a film and/or the degree of film formation in the art may be used. Such procedures may be used to empirically characterize a film, and determine whether a coating composition produces a film suitable for a given application. Flexibility is the film's ability to undergo stress from bending and/or flexing without discernable damage (e.g., cracking). Examples of a standard technique for determining the flexibility of a film under mechanical or temperature stress are described in “ASTM Book of Standards, Volume 06.01, Paint—Tests for Chemical, Physical, and Optical Properties; Appearance,” D522-93a and D4145-83, 2002; “ASTM Book of Standards, Volume 06.02, Paint—Products and Applications; Protective Coatings; Pipeline Coatings,” D4145-83, D4146-96, and D1211-97, 2002; and “Paint and Coating Testing Manual, Fourteenth Edition of the Gardner-Sward Handbook,” (Koleske, J. V. Ed.), pp. 547-554, 1995. Related to flexibility is the tensile strength of a film, which is the ability of a film to undergo tensile deformation without developing discernable damage (e.g., cracking, tearing). Examples of a standard technique for determining the tensile strength of a film are described in “ASTM Book of Standards, Volume 06.01, Paint—Tests for Chemical, Physical, and Optical Properties; Appearance,” D2370-98 and D522-93a, 2002; and “Paint and Coating Testing Manual, Fourteenth Edition of the Gardner-Sward Handbook,” (Koleske, J. V. Ed.), pp. 534-545, 1995. Toughness is the film's ability to undergo strain imposed in a short period of time (e.g., one second or less) without discernable damage (e.g., breaking, tearing). Examples of a standard technique for determining the toughness of a film (e.g., a film for a pipeline) are described in “ASTM Book of Standards, Volume 06.01, Paint—Tests for Chemical, Physical, and Optical Properties; Appearance,” D2794-93, 2002; “ASTM Book of Standards, Volume 06.02, Paint—Products and Applications; Protective Coatings; Pipeline Coatings,” G14-88, 2002; and “Paint and Coating Testing Manual, Fourteenth Edition of the Gardner-Sward Handbook,” (Koleske, J. V. Ed.), pp. 547-554, 1995. Impact resistance is the ability of a film to undergo impact with an indenter without developing discernable damage at the dimple site (e.g., cracking). Examples of a standard technique for determining the impact resistance of a film (e.g., a film for a pipeline) are described in “ASTM Book of Standards, Volume 06.01, Paint—Tests for Chemical, Physical, and Optical Properties; Appearance,” D2794-93, 2002; “ASTM Book of Standards, Volume 06.02, Paint—Products and Applications; Protective Coatings; Pipeline Coatings,” G13-89 and G14-88, 2002; and “Paint and Coating Testing Manual, Fourteenth Edition of the Gardner-Sward Handbook,” (Koleske, J. V. Ed.), pp. 553-554, 1995. Hardness is the film's ability to undergo an applied static force without developing discernable damage (e.g., a scratch, an indentation). Examples of a standard technique for determining the hardness of a film are described in “ASTM Book of Standards, Volume 06.01, Paint—Tests for Chemical, Physical, and Optical Properties; Appearancei” D1640-95, D1474-98, D2134-93, D4366-95, and D3363-00, 2002; and “Paint and Coating Testing Manual, Fourteenth Edition of the Gardner-Sward Handbook,” (Koleske, J. V. Ed.), pp. 555-584, 1995. Mar resistance (“mar abrasion resistance”) is the film's ability to undergo an applied dynamic force without developing a change in the film surface appearance (e.g., gloss) due to a permanent deformation (e.g., an indentation). Examples of a standard technique for determining the mar resistance of a film are described in “ASTM Book of Standards, Volume 06.01, Paint—Tests for Chemical, Physical, and Optical Properties; Appearance,” D5178-98 and D6037-96, 2002; and “Paint and Coating Testing Manual, Fourteenth Edition of the Gardner-Sward Handbook,” (Koleske, J. V. Ed.), pp. 525-533 and 579-584, 1995. Abrasion resistance (“wear abrasion resistance”) is the film's ability to undergo an applied dynamic force (e.g., washing) without removal of film material. Examples of a standard technique for determining the abrasion resistance (e.g., burnish resistance) of a film are described in “ASTM Book of Standards, Volume 06.01, Paint—Tests for Chemical, Physical, and Optical Properties; Appearance,” D968-93 and D4060-01, 2002; “ASTM Book of Standards, Volume 06.02, Paint—Products and Applications; Protective Coatings; Pipeline Coatings,” D3170-01, D4213-96, D5181-91, D4828-94, D2486-00, D3450-00, D6736-01, and D6279-99e1, 2002; and “Paint and Coating Testing Manual, Fourteenth Edition of the Gardner-Sward Handbook,” (Koleske, J. V. Ed.), pp. 525-533, 1995. Blocking resistance (“block resistance”) is the ability of a film to resist adhering to a second film, particularly when the two films are pressed together (e.g., a coated door and coated doorframe). Examples of a standard technique for determining the blocking resistance of a film are described in “ASTM Book of Standards, Volume 06.02, Paint—Products and Applications; Protective Coatings; Pipeline Coatings,” D2793-99 and D3003-01, 2002. Abrasion resistance (“wear abrasion resistance”) is the film's ability to undergo an applied dynamic force (e.g., washing) without removal of film material. Slip resistance is a coating's (e.g., a floor coating) slipperiness, and can be evaluated as described in “Paint and Coating Testing Manual, Fourteenth Edition of the Gardner-Sward Handbook,” (Koleske, J. V. Ed.), pp. 600-606, 1995.

Weathering resistance is film's ability to endure and/or protect a surface from an external environmental condition. Examples of environmental conditions that may damage a film and/or surface include contact with varying conditions of temperature, moisture, sunlight (e.g., UV resistance), pollution, biological organisms, or a combination thereof. Examples of a standard technique for determining the weathering resistance of a film (e.g., an automotive film, an external architectural film, a varnish, a wood coating, a steel coating) by evaluating the degree of damage (e.g., fungal growth, color alteration, dirt accumulation, gloss loss, chalking, cracking, blistering, flaking, erosion, surface rust), are described in “ASTM Book of Standards, Volume 06.01, Paint—Tests for Chemical, Physical, and Optical Properties; Appearance,” D4141-01, D1729-96, D660-93, D661-93, D662-93, D772-86, D4214-98, D3274-95, D714-02, D1654-92, D2244-02, D523-89, D1006-01, D1014-95, and D1186-01, 2002; “ASTM Book of Standards, Volume 06.02, Paint—Products and Applications; Protective Coatings; Pipeline Coatings,” D3719-00, D610-01, D1641-97, D2830-96, and D6763-02, 2002; and “Paint and Coating Testing Manual, Fourteenth Edition of the Gardner-Sward Handbook,” (Koleske, J. V. Ed.), pp. 619-642, 1995. Additionally, standard techniques in the art for determining the resistance of a film to artificial weathering conditions may be used. These procedures are used to contact a film with a simulated weathering condition (e.g., heat, moisture, light, UV irradiation) at an accelerated timetable are described in “ASTM Book of Standards, Volume 06.01, Paint—Tests for Chemical, Physical, and Optical Properties; Appearance,” D822-01, D4587-01, D5031-01, D6631-01, D6695-01, D5894-96, and D4141-01, 2002; “ASTM Book of Standards, Volume 06.02, Paint—Products and Applications; Protective Coatings; Pipeline Coatings,” D5722-95, D3361-01 and D3424-01, 2002; and “Paint and Coating Testing Manual, Fourteenth Edition of the Gardner-Sward Handbook” (Koleske, J. V. Ed.), pp. 643-653, 1995.

Standard techniques for determining a film's resistance to damage by various chemicals in the art may be used. Examples of chemicals that can be used in such procedures include an acid (e.g., 3% acetic acid), a base, an alcohol (e.g., 50% ethyl alcohol, hydrochloric acid, sulfuric acid), a detergent (e.g., a sodium phosphate solution), gasoline, a glycol based antifreeze, an oil (e.g., a vegetable oil, a lubricating petroleum oil, a grease), a solvent, water (e.g., a salt solution, a salt vapor), a polish abrasive, another coating (e.g., graffiti), or a combination thereof. Standard techniques for determining the chemical resistance of a film (e.g., an architectural film, an automotive film, a paint, a lacquer, a varnish, a traffic-coating, a metal surface-film) by evaluating possible damage (e.g., adhesion loss, alteration of gloss, blistering, discoloration, loss of hardness, staining, swelling, wrinkling) are described in, for example, “ASTM Book of Standards, Volume 06.02, Paint—Products and Applications; Protective Coatings; Pipeline Coatings,” D1308-02, D2571-95, D2792-69, D4752-98, D3260-01, D6137-97, D6686-01, D6688-01, and D6578-00, 2002; “ASTM Book of Standards, Volume 06.01, Paint—Tests for Chemical, Physical, and Optical Properties; Appearance,” D2370-98, D2248-Ola, and D870-02, 2002; “ASTM Book of Standards, Volume 06.03, Paint—Pigments, Drying Oils, Polymers, Resins, Naval Stores, Cellulosic Esters, and Ink Vehicles,” D1647-89, 2002; and “Paint and Coating Testing Manual, Fourteenth Edition of the Gardner-Sward Handbook,” (Koleske, J. V. Ed.), pp. 662-666, 1995. Additionally, examples of a standard technique for determining the solvent resistance of a film are described in “ASTM Book of Standards, Volume 06.02, Paint—Products and Applications; Protective Coatings; Pipeline Coatings,” D4752-98 and D5402-93, 2002.

Standard techniques for determining a film's and/or surface's (e.g., metal, wood) resistance to water permeability and/or damage (e.g., corrosion, blistering, adhesion reduction, hardness alteration, color alteration, gloss alteration) by contact with water and/or moisture are described in, for example, “ASTM Book of Standards, Volume 06.01, Paint—Tests for Chemical, Physical, and Optical Properties; Appearance,” D870-02, D1653-93, D1735-02, D2247-02, and D4585-99, 2002; and “ASTM Book of Standards, Volume 06.02, Paint—Products and Applications; Protective Coatings; Pipeline Coatings,” D2065-96, D2921-98, D3459-98, and D6665-01, 2002.

Standard techniques for determining a film's resistance to damage by a temperature greater than ambient condition in the art may be used. Thermal resistance is the film's ability to undergo stress from a temperature at or below 200° C. without discernable damage, while heat resistance is the film's ability to undergo stress from a temperature above 200° C. (e.g., fire resistance, fire retardancy, flame resistance) without discernable damage. Standard techniques for determining the thermal and/or heat resistance of a film (e.g., a metal-film, a wood-lacquer) by evaluating possible damage (e.g., adhesion loss, alteration of gloss, blistering, chalking, discoloration) are described in, for example, “ASTM Book of Standards, Volume 06.01, Paint—Tests for Chemical, Physical, and Optical Properties; Appearance,” D2370-98, D2485-91, D1360-98, D4206-96, and D3806-98, 2002; and “ASTM Book of Standards, Volume 06.02, Paint—Products and Applications; Protective Coatings; Pipeline Coatings,” D1211-97 and D6491-99, 2002.

In some embodiments, it may be desirable to measure the component composition of a coating and/or film such as to verify the presence, absence and/or amount of one or more coating components in a particular formulation. Standard procedures for sampling a coating and/or film, and analyzing the material composition (e.g., a pigment, a binder, liquid component, toxic material), have been described in, for example, “ASTM Book of Standards, Volume 06.01, Paint—Tests for Chemical, Physical, and Optical Properties; Appearance,” D2371-85, D5380-93, D2372-85, D2698-90, D3723-84, D4451-02, D4563-02, D5145-90, D3925-02, D2348-02, D2245-90, D3624-85a, D3717-85a, D2349-90, D2350-90, D2351-90, D2352-85, D3271-87, D3272-76, D4017-02, D3792-99, D4457-02, D6133-00, D6191-97, D4764-01, D3718-85a, D3335-85a, D6580-00, E848-94, D4834-88, D4358-84, D2621-87, D3618-85a, D6438-99, D4359-90, D3168-85, and D4948-89, 2002; “ASTM Book of Standards, Volume 06.02, Paint—Products and Applications; Protective Coatings; Pipeline Coatings,” D5702-02, 2002; and “ASTM Book of Standards, Volume 06.03, Paint—Pigments, Drying Oils, Polymers, Resins, Naval Stores, Cellulosic Esters, and Ink Vehicles,” D1469-00, 2002.

The nonvolatile content of a coating component and/or coating (“total solids content”) can provide an estimate, for example, of the volume of film that will be produced by a coating or coating component (e.g., a paint, a clear coating, an electrocoat bath applied coating, a binder solution, an emulsion, a varnish, an oil, a drier, a solvent) and/or the surface area a coating can cover relative to a film's thickness. The nonvolatile content of coating and/or coating component can be determined by any technique known in the art (see, for example, “ASTM Book of Standards, Volume 06.01, Paint—Tests for Chemical, Physical, and Optical Properties; Appearance,” D6093-97, D2697-86, D1259-85, D1644-01, D2832-92, and D4209-82 D5145-90, 2002; “ASTM Book of Standards, Volume 06.02, Paint—Products and Applications; Protective Coatings; Pipeline Coatings,” D4713-92, D5095-91, 2002; and “ASTM Book of Standards, Volume 06.03, Paint—Pigments, Drying Oils, Polymers, Resins, Naval Stores, Cellulosic Esters, and Ink Vehicles,” D4139-82, 2002. Additionally, the volatile component of a coating can provide an estimate, for example, of VOC release and/or thermoplastic film formation time. The nonvolatile content of coating and/or coating component (e.g., a paint, a clear coating, an automotive coating, an emulsion, a binder solution, a varnish, an oil, a drier, a solvent) can be determined by any technique known in the art (see, for example, “ASTM Book of Standards, Volume 06.01, Paint—Tests for Chemical, Physical, and Optical Properties; Appearance,” D2369-01e1, D2832-92, D3960-02, D4140-82, D4209-82, D5087-02 and D6266-00a, 2002; and “ASTM Book of Standards, Volume 06.02, Paint—Products and Applications; Protective Coatings; Pipeline Coatings,” D5403-93, 2002.

Standard procedures for determining the visual appearance of a coating component, coating and or film (e.g., reflectance, retroreflectance, fluorescence, photoluminescent light transmission, color, tinting strength, whiteness, measurement instruments, computerized data analysis) have been described, for example, in “ASTM Book of Standards, Volume 06.01, Paint—Tests for Chemical, Physical, and Optical Properties; Appearance,” E284-02b, E312-02, E805-Ola, E179-96, E991-98, E1247-92, E308-01, E313-00, E808-01, E1336-96, E1341-96, E1347-97, E1360-90, D332-87, D387-00, E1455-97, E1477-98a, E1478-97 E1164-02, E1331-96, E1345-98, E1348-02, E1349-90, D5531-94, D3964-80, E1651-94, E1682-96, E1708-95, E1767-95, E1808-96, E1809-01, E2022-01, E2072-00, E2073-02, E2152-01, E2153-01, D1544-98, E259-98, D3022-84, D1535-01, E2175-01, E2214-02, and E2222-02, 2002; “ASTM Book of Standards, Volume 06.02, Paint—Products and Applications; Protective Coatings; Pipeline Coatings,” D4838-88 and D5326-94a, 2002; and “ASTM Book of Standards, Volume 06.03, Paint—Pigments, Drying Oils, Polymers, Resins, Naval Stores, Cellulosic Esters, and Ink Vehicles,” D2090-98, D2090-98 and D6166-97, 2002. Specific techniques for matching two or more colored coatings and/coating components to minimalize differences (e.g., metamerism) have been described, for example, in “ASTM Book of Standards, Volume 06.01, Paint—Tests for Chemical, Physical, and Optical Properties; Appearance,” D4086-92a, E1541-98 D2244-02 2002. Specific techniques for determining differences in the color of a coatings and/coating components, particularly to insure color consistency of a coating composition, “ASTM Book of Standards, Volume 06.01, Paint—Tests for Chemical, Physical, and Optical Properties; Appearance,” D1729-96, D2616-96, E1499-97, and D3134-97, 2002.

Gloss is the film's “angular selectivity of reflectance, involving surface-reflected light, responsible for the degree to which reflected highlights or images of objects may be seen as superimposed on a surface” (“ASTM Book of Standards, Volume 06.01, Paint—Tests for Chemical, Physical, and Optical Properties; Appearance,” E284-02b, 2002). An example of a high gloss coating would be a paint film with a glass-like surface apparance, as opposed to a low-gloss (“flat”) paint. Standard techniques for determining the gloss (e.g., specular gloss, sheen, haze, image clarity, waviness, directionality) of a coating and/or film are described, for example, in “ASTM Book of Standards, Volume 06.01, Paint—Tests for Chemical, Physical, and Optical Properties; Appearance,” E284-02b, D523-89, D4449-90, E167-96, E430-97, D4039-93, D5767-95, and D2244-02, 2002; “ASTM Book of Standards, Volume 06.02, Paint—Products and Applications; Protective Coatings; Pipeline Coatings,” D3928-00a, 2002; and “Paint and Coating Testing Manual, Fourteenth Edition of the Gardner-Sward Handbook,” (Koleske, J. V. Ed.), pp. 470-480, 1995.

7. Additional Enzyme Uses of the Invention

In certain embodiments, the compositions and methods that comprise a biomolecular composition with organophosphorus compound degradation ability will have use in three primary markets that will benefit from a susceptible surface covered with a self-decontaminating coating: domestic military, friendly foreign military/civilian, and domestic civilian. It is contemplated that for military use, a self-decontaminating coating has utility on a surface of a vehicle, a trailer, a barrack, a decontamination shelter, a piece of equipment (e.g., a piece of electronic equipment) or a combination thereof.

It is further contemplated that a biomolecular composition may have dual military and/or civilian use in a method for facilitating the disposal of a chemical waste, including but not limited to, a CWA, a pesticide or a combination thereof. A particular dual use embodiment includes coating a surface that may be in a facility where there would be an unexceptable delay to the use of a piece of equipment, a space (e.g., a room, a command center, a computer center), a vehicle (e.g., a public transportation vehicle, an emergency vehicle) or a combination thereof if the facility was subjected to or suspected of exposure to, a dangerous chemical (e.g., a nerve agent). In some aspects, the piece of equipment, the space, and/or the vehicle may be used by a military personnel, an emergency personnel or a combination thereof. It is contemplated that such a facility may be contacted with a chemical from a chemical weapon attack (e.g., a CWA gas attack), an accidental release of a chemical, or a combination thereof. Examples of such facilities include a control room at a military base, an airport, a nuclear power plant, a hospital, or a combination thereof. It is an aspect of the disclosure that a facility (i.e., a space, a vehicle, a piece of equipment) that may be subject to exposure to a chemical (e.g., a nerve agent) may be coated with the disclosed compositions and would then be detoxified and safe after contact with the chemical.

Civilian applications contemplated include a coating of a surface in contact with air, such as for example, a ventilation intake or an air filter, as well as a surface (e.g., an interior surface, an exterior surface) comprised in a hospital clean room, a community safe room, a control room for a nuclear plant, a control room for a chemical plant, a control room for a power plant, a control room for a water plant, a government building, an industrial building, a facility for public transportation (e.g., a train, a subway, a plane, an airport), and a surface of an equipment by a first responder, or any combination of the forgoing.

It is contemplated that for each formulation of a coating and a biomolecular composition, enzymatic decontamination parameters based on chemical (e.g., CWA simulant) degradation assessment will be established in a range of exterior weathering conditions. If a specific formulation of enzyme composition in a coating remains active after exposure to exterior weathering conditions, there is a significant utility for using the bioactive painted surfaces in exterior and field application. For example, it is contemplated that in some embodiments a biomolecular composition incorporated in standard formulations of water-based or latex-based paint will result in minimal to no changes in the durability of the paint based on standard exterior weathering conditions. In a general aspect, a weathering study may indicate a need to reformulate a composition to improve a particular property (e.g., enhance biomolecular composition stability). In this aspect, it is contemplated that standard methods, known to those of skill in the art (e.g., encapsulation), may be used to increase stability and re-test the resulting formulation. Application of such methods can be used to modify various formulations to produce a composition with one or more properties suited for a particular application, as described herein and as understood in the art in light of the present disclosures.

8. Additional Enzyme Uses—Combinations of Decontamination Compositions and Methods

In certain embodiments, a composition or method that possesses an organophosphorus degradation ability may be combined with another composition method for decontamination (e.g., detoxification, degradation) of a chemical. In some aspects, the additional composition or method comprises one for decontamination of a pesticide or chemical warfare agent. Such additional compositions and methods are known in the art (Yang, Y. C. et al., 1992), and may be applied prior, during and/or after application of a composition and/or method. In particularly additional embodiments, such a combination of a composition and/or method disclosed herein with a traditional composition and/or method produces greater decontamination than that achieved without such a combination.

Additional compositions that are contemplated include, but are not limited to, a caustic agent; a decontaminating foam (e.g., Sandia, Decon Green); an application of intensive heat and carbon dioxide for a sustained period; an incorporation of a material into a coating that, when exposed to sustained high levels of UV light, degrades a chemical; a chemical agent resistant coating; or a combination thereof. Examples of a caustic agent include, a bleaching agent, DS2, or a combination thereof.

As used herein, a “caustic agent” is a composition capable of destroying usually via a chemical reaction, a material, unfortunately including animal tissue such as skin. Thus, application of a caustic agent is often accompanied by the wearing of protective gear for those not contaminated or suspected of being contaminated. Certain caustic agents, such as for example, a bleaching agent or decontamination solution 2 (“DS2”), have specifically been formulated and/or used to decontaminate chemical warfare agents. Both G agents and VX can be decontaminated with these caustic agents. As used herein, a “bleaching agent” refers to a reactive chemical compound capable breaking a double bond in another chemical compound, which is often a useful property for degrading a toxic or otherwise undesirable chemical. Examples of a bleaching agent include a bleach powder, a bleach solution, or a combination thereof. A bleach powder may comprise, but is not limited to, Ca(OCl)Cl and Ca(OCl)₂ (“high test hypochlorite,” “HTH”); Ca(OCl)₂ and CaO (“super tropical bleach,” “STB”); Ca(OCl)₂ and MgO (“Dutch powder”); or a combination thereof. A bleach solution may comprise, but is not limited to, NaOCl (“bleach”), usually 2% to 6% wt in water; a HTH slurry, usually 7% HTH wt in water; a STB slurry, usually 7% to 70% wt in water; activated solution of hypochlorite (“ASH”), usually 0.5% Ca(OCl)₂ and 0.5% sodium dihydrogen phosphate buffer and 0.05% detergent in water; self-limited activated solution of hypochlorite (“SLASH”), usually 0.5% Ca(OCl)₂ and 1.0% sodium citrate and 0.2% citrate acid and 0.05% detergent in water; or a combination thereof. Bleach, Dutch powder, ASH and SLASH are generally applied to skin and equipment for decontamination, while HTH and STB are generally applied to equipment and terrain for decontamination. VX is may be decontaminated at an acid pH, wherein it is more soluble (Yang, Y. C. et al., 1992).

DS2 was developed to function at various temperatures (i.e., −25° C. to 52° C.), particularly those below the freezing point of many aqueous compositions. It usually comprises 70% diethylenetriamine (H₂NCH₂CH₂NHCH₂CH₂NH₂), 28% ethylene glycol monomethyl ether (CH₃OCH₂CH₂OH), and 2% sodium hydroxide (NaOH). DS2 is noncorrosive to many metals, but is damaging to many paints, leathers, rubber materials, plastics and skin. Contact with a paint is generally limited to 30 minutes or less. An aqueous rinse is generally used to remove DS2, and exposure to air and/or water degrades DS2 (Yang, Y. C. et al., 1992).

Various other decontamination compositions and methods are known to those of skill in the art. Examples of a decontaminating foam include Sandia, Decon Green, or a combination thereof. Examples of an incorporation of a material include incorporation of TiO₂ and porphyrins into acetonitrile coatings that, when exposed to a sustained high level of UV light in an oxygen environment (e.g., air), degrade a chemical agent (e.g., mustard). Addition of water to the acetonitrile coating comprising TiO₂ and porphyrins will aid the degradation of VX to non-toxic compounds (Buchanan, J. H. et al., 1989; Fox, M. A., 1983). Additionally, CARCs have been developed to withstand repeated decontamination efforts.

Decontamination compositions are often prepared and packaged in equipment for easy of handling. Such an equipment packages include, but are not limited to, kits (e.g., a towelette package), and delivery apparatus (e.g., a sprayer). Examples of specific decontamination equipment packages that may be used in combination with a composition or method include a ABC-M11 portable decontamination apparatus, which comprises DS2, a devise for spraying DS2, and a vehicle mounting bracket; a ABC-M12A1 power-driven, skid-mounted decontamination apparatus, which comprises a personnel shower unit, a pump, a tank, a M2 water heater, and delivers water, foam, DS2, STB, and/or deicing liquid; a M258A1 personal decontamination kit, which comprises towelettes soaked with a decontamination solution (i.e., 72% ethanol, 10% phenol, 5% NaOH, 0.2% ammonia, and 12% water), ampules of a decontaminating solution (5% ZnCl₂, 45% ethanol, 50% water) for adding to a towlette soaked with chloramines-B (PhS(O)₂NClNa), packing foil, and a plastic carrying case; a M280 individual equipment decontamination kit, which comprises twenty fold the contents of the M258A1 kit; a M291 skin decontamination kit, which comprises six XE-555 resin (i.e., styrene/divinyl benzene copolymer, a strong acid cation-exchange resin and a strong base anion-exchange resin for absorption and chemical detoxification) filled fiber pads packaged in foil; a M13 portable decontamination apparatus, which comprises DS2, a container and an equipment/vehicle mount, and is capable of dispensing DS2; a M17 lightweight, transportable decontamination apparatus, which comprises hoses, cleaning jets, personnel showers, a collapsible rubberized fabric tank, and is capable of dispensing water; or a combination thereof. The ABC-M11, M13 and M280 decontamination equipment packages are generally used for equipment (e.g., vehicles), the M258A1 and M17 decontamination equipment packages are generally used for equipment and/or personnel, and the ABC-M12A1 and M291 decontamination equipment packages are generally used for personnel (Yang, Y. C. et al., 1992).

9. Removing a Coating or Film

In certain embodiments, it may be desirable to remove a coating and/or film from a surface such as a non-film forming coating, a temporary film, a self-cleaning film, a coating and/or film that has been damaged, or is otherwise no longer desired or no longer is suitable for use. Various coating removers (e.g., a paint remover) in the art may be used, and often comprise solvents described herein capable of dissolving a coating component (e.g., a binder) integral to a film's structural integrity. Standard procedures for determining the effectiveness of a coating remover have been described, for example, in “ASTM Book of Standards, Volume 06.02, Paint—1Products and Applications; Protective Coatings; Pipeline Coatings,” D6189-97, 2002.

The general effectiveness of various embodiments is demonstrated in the following Examples. Some methods for preparing compositions are illustrated. Starting materials are made according to procedures known in the art or as illustrated herein. The following Examples are provided so that the embodiments might be more fully understood. These Examples are illustrative only and should not be construed as limiting in any way, as other coating formulations (e.g., a different paint formulation) or polymeric materials comprising different biomolecular compositions (e.g., a different purified or partly purified enzyme, a different cell-based particulate material comprising an enzyme) may be prepared.

### EXAMPLE 1

This Example demonstrates the effectiveness of lysozyme in lysing the bacterium Micrococcus lysodeikticus. M. lysodeikticus was used as a lysozyme substrate in a liquid suspension in the assay. The assay measured the rate of decrease in the absorbance as a relative measure of the amount/availability/activity of a lysozyme present in a material. As cell lysis occurs, the turbidity of a cell suspension decreased, and therefore, the absorbance of a cell suspension decreased. Materials and reagents that were used are shown in the table below.

TABLE 9
Materials and Reagents

2 M sodium phosphate buffer (NaH₂PO₄), pH 6.4, or Tris-HCL
Buffer, pH 7.0
Micrococcus lysodeikticus cell (Worthington Biochemicals, #8736)
Lysozyme (chicken egg white) (Sigma Product #L 6876,
CAS 12650-88-3)
96-well plate
Thermo Multiskan Ascent Plate Reader
Pipettes and Pipetteman
Microtubes

The reagents that were prepared included a M. lysodeikticus cell suspension comprising 9 mg M. lysodeikticus in 25 mL sodium phosphate buffer, and a lysozyme solution comprising a 5 mg/mL stock solution.

The assay procedure included diluting the lysozyme stock solution with buffer to create the following samples: 5 mg/mL (undiluted); 2.5 mg/mL; 1 mg/mL; 0.5 mg/mL; 0.1 mg/mL; 0.05 mg/mL; 0.01 mg/mL; 0.005 mg/mL; 0.001 mg/mL; 0.0005 mg/mL; 0.0001 mg/mL; and 0.00005 mg/mL. Control samples included: 3 replicates of 200 μL M. lysodeikticus cell suspension and 3 replicates of 200 μL buffer that were pipetted into 6 wells total in a 96-well microplate. A 194 μL Micrococcus cell suspension was pipetted into 3 rows of 12 wells each. 6 μL of each lysozyme concentration assayed was then added to the M. lysodeikticus cell suspension using a multi-pipette and mixed. The plate was immediately placed into the Thermo Multiskan Ascent Plate Reader; each well was read every 10 seconds for 30 minutes to determine the absorbance at 450 nm.

TABLE 10
Lysis of M. lysodeikticus (Ml) over a concentration range of
lysozyme

| Lysozyme | | Time | | | Ml lysed |
|---|---|---|---|---|---|
| (mg × 10⁻³) | Abs | (sec) | dAbs | dAbs/sec | (mg × 10⁻⁶)/sec |
| 0.01 | 0.37 | 1800 | 0.015 | 8.33 × 10⁻⁶ | 1.6 |
| 0.02 | 0.35 | 1800 | 0.035 | 1.94 × 10⁻⁵ | 3.6 |
| 0.1 | 0.31 | 1800 | 0.075 | 4.17 × 10⁻⁵ | 7.8 |
| 0.2 | 0.22 | 1800 | 0.165 | 9.17 × 10⁻⁵ | 17.1 |
| 1 | 0.275 | 300 | 0.11 | 3.67 × 10⁻⁴ | 68.6 |
| 2 | 0.13 | 520 | 0.255 | 4.9 × 10⁻⁴ | 91.7 |
| 10 | 0.26 | 2 | 0.125 | 6.25 × 10⁻² | 11688.3 |
| 20 | 0.23 | 2 | 0.155 | 7.75 × 10⁻² | 14493.5 |
| 100 | 0.165 | 2 | 0.22 | 1.1 × 10⁻¹ | 20571.4 |

TABLE 11
Summary of Activity

| | |
|---|---|
| Abs | 0.38 |
| [Ml] | 0.36 mg/ml |
| Vol | 0.2 ml |
| | 0.187 dmg/dOD |
| Rate | 0.047 dmg Ml/sec/mg |
| | lysozyme |

The results for the lysozyme assay under the conditions as described: 1 mg of lysozyme was able to lyse 0.047 mg of M. lysodeikticus per sec. The lysozyme was effective in lysing M. lysodeikticus cells, and these results were consistent under both conditions evaluated (Tris vs NaH₂PO₄)

### EXAMPLE 2

This Example demonstrates the ability of a lysozyme to survive the incorporation process into a coating, demonstrates lysozyme hydrolytic activity in a coating environment, and demonstrates the ability of lysozyme to survive in can conditions for 48 hours. A Sherwin-Williams Acrylic Latex paint was used. Materials, reagents and equipment used are shown in the tables below.

TABLE 12
Materials and Reagents

0.1 M potassium phosphate buffer, pH 6.4
Micrococcus lysodeikticus (Worthington Biochemicals, #8736)
Sherwin-Williams Acrylic Latex paint
Lysozyme (chicken egg white) (Sigma Product #L 6876,
CAS 12650-88-3)
15 mL plastic test tubes

TABLE 13
Equipment

Paint spreader (1-8 mil)
Polypropylene blocks
Lightnin Labmaster Mixer
Rotator shaker
Pipettes and Pipetteman
Klett-Sumerson Colorimeter (Filter D35: 540 nm)

The reagents prepared included a Micrococcus cell suspension comprising 9 mg M. lysodeikticus in 25 mL sodium phosphate buffer, and a lysozyme solution comprising a 5 mg/mL stock solution. The paint formulations used are shown in the table below.

TABLE 14
Paint Preparation

Sherwin-Williams Acrylic Latex Control (no additive)
Sherwin-Williams Acrylic Latex with 1 mg/mL lysozyme

The paint was mixed with a glass stirring rod and a paint mixer. Each film was immediately drawn onto polypropylene surfaces with a thickness of 8 mil. Cure time for the Sherwin-Williams was 72 hrs. To demonstrate in can durability, the Sherwin-Williams Acrylic Latex comprising lysozyme wet paint was sealed and shelf stored at ambient temperature. After 48 hrs in can, films were drawn onto polypropylene surfaces with a thickness of 8 mils and were allowed to cure 72 hrs prior to assay. Coupons were generated as free films from the polypropylene surface. Films were generated in three sizes: 2 cm²: 1 cm by 2 cm; 4 cm²: 1 cm by 4 cm; or 6 cm²: 1 cm by 6 cm.

For qualitative assessment, individual films were placed into labeled 15 mL tubes. Films of each size (2, 4 and 6 cm²) were evaluated in triplicate. In addition to a control paint with no additive, two other controls were utilized, a positive control and a negative control. The positive control comprised: lysozyme in buffer added to each of three 15 mL tubes in concentrations approximating the amount of lysozyme in the films (i.e., 40 μg, 80 μg, and 120 μg). Each amount was assayed in triplicate. The negative control comprised: 5 mL of 0.36 mg/mL M. lysodeikticus cell suspension pipetted into a single 15 mL tube. 5 mL 0.36 mg/mL Micrococcus lysodeikticus cell suspension was added to all reaction tubes to begin the reaction. The tubes were placed on a rocker at ambient conditions for approximately 22 hours. Where possible, the films were removed from the suspension and determine opacity using the Klett-Summerson Colorimeter (turbidity unit: Klett Unit or KU).

Particulate matter in the samples interfered with quantitation; photographs of each set of 2 cm² paint films and controls following 22 hour contact to M. lysodeikticus cell suspension were taken, and observations recorded in the Tables below.

TABLE 15
Qualitative Observations (visual assessments)

| Sample¹ | Lysozyme (μg) | Film Size (cm²) | Clarity |
|---|---|---|---|
| Suspension/Solution | | | |
| Controls | | | |
| M. lysodeikticus | — | — | Translucent |
| Lysozyme | 40 | — | Transparent² |
| | 80 | — | Transparent |
| | 120 | — | Transparent |
| Control Films | | | |
| S-W | | 2, 4, 6 | Translucent |
| Films Comprising | | | |
| Lysozyme | | | |
| S-W | | 2, 4, 6 | Transparent |

¹Each evaluation was performed in triplicate
²Thinned in opacity, with some suspended particulate matter

The strips comprising lysozyme of all three sizes of coupons cleared the M. lysodeikticus suspension, indicating that the lysozyme maintains activity in the coating environment. Cleared suspensions (lysozyme comprising coupons and controls) comprised large particles which interfere with the quantitation of the cleared suspensions. The particulate matter was less obvious in the 2 cm² set comprising lysozyme, so this size coupon was used for the quantitative demonstrations.

TABLE 16
Quantiative Assessment of Lysozyme In-Film Activity
(2 cm² film, 4 hr time point, 3 independent assays,
each performed in triplicate.)

| | | Replicate 1 | | Replicate 2 | | Replicate 3 | |
|---|---|---|---|---|---|---|---|
| | In can | | Cell | | Cell | | Cell |
| Formulation | (hrs) | KU | lysis | KU | lysis | KU | lysis |
| Suspension Controls | | | | | | | |
| M. lysodeikticus | | 81.5 | 0.0% | 101 | 0% | | |
| Lysozyme | | 17 | | 27 | | | |
| S-W Acrylic Latex | | | | | | | |
| Control Films | — | 75 | 18% | 74 | 19% | 71 | 22% |
| | — | 79 | 13% | 82 | 10% | 76 | 17% |
| | — | 83 | 9% | 81 | 11% | 73 | 20% |
| Films Comprising | — | 8 | 91% | 20 | 78% | 11 | 88% |
| Lysozyme | — | 13 | 86% | 11 | 88% | 15 | 84% |
| | — | 13 | 86% | 5 | 95% | 0 | 100% |
| Control Films | 48 hrs | 82 | 10% | 65 | 29% | 68 | 25% |
| Films Comprising | 48 hrs | 36 | 61% | 26 | 72% | 37 | 59% |
| Lysozyme | | | | | | | |

KU = Klett Units, measure of turbidity at 540 nm.

A lysozyme in Sherwin-Williams Acrylic Latex was able to lyse about 88% of theM. lysodeikticus culture over 4 hours, relative to the control which exhibited about a 15% drop in opacity. After in-can shelving for 48 hrs (i.e., the lysozyme was mixed into the Sherwin-Williams Acrylic Latex, capped and shelved for 48 hrs prior to drawing down the films), the lysozyme remained active, lysing about 64% of the M. lysodeikticus culture relative to the about 21% lysis exhibited by the control panels.

### EXAMPLE 3

This Example demonstrates the retention of lysozyme vs. loss due to leaching in a paint film in a saturated condition at 1, 2 and 24 hours after submersion. Materials, reagents and equipment used are shown in the tables below.

TABLE 17
Materials and Reagents

0.1 M potassium phosphate buffer, pH 6.4
Micrococcus lysodeikticus (Worthington Biochemicals, #8736)
Lysozyme (chicken egg white) (Sigma Product #L 6876,
CAS 12650-88-3)
Sherwin-Williams Acrylic Latex paint
15 mL plastic test tubes

TABLE 18
Equipment

Paint spreader (1-8 mil)
Polypropylene blocks
Lightnin Labmaster Mixer
Rotator shaker
Pipetter and tips
Klett-Sumerson Colorimeter (Filter D35: 540 nm)

The reagents prepared included a Micrococcus cell suspension comprising 9 mg M. lysodeikticus in 25 mL sodium phosphate buffer, and a lysozyme solution comprising a 5 mg/mL stock solution.

The paint formulations that were prepared included a Sherwin-Williams Acrylic Latex Control (no additive), and a Sherwin-Williams Acrylic Latex comprising 1 mg/mL lysozyme. Each paint was mixed with a glass stirring rod and a paint mixer. Each film was immediately drawn onto polypropylene surfaces with a thickness of 8 mil. Cure time was 120 hrs. The Sherwin-Williams Acrylic Latex comprising a lysozyme wet paint was sealed and shelf stored at ambient temperature. After 48 hrs in can storage, films were drawn onto polypropylene surfaces with a thickness of 8 mils and were allowed to cure 72 hrs prior to assay. Materials for assay were generated from the polypropylene surface as a 2 cm² (1×2 cm) free film.

The assay procedure included placing individual films into labeled 15 mL tubes. 24 hours prior to addition of Micrococcus lysodeikticus cell suspension, 5 mL KPO₄ buffer was added to the 24-hour control and coupon comprising a lysozyme tube, as well as one tube comprising 41 μg lysozyme solution (positive control) and one tube comprising 5 mL of the M. lysodeikticus cell suspension (negative control). These tubes were placed on the shaker for 24 hrs.

2 hours prior to addition of M. lysodeikticus, 5 mL potassium phosphate buffer was added to the 2-hour control and lysozyme tubes each comprising a coupon, as well as one tube comprising 41 μg lysozyme solution (positive control) and one tube comprising 5 mL of the M. lysodeikticus cell suspension (negative control). These tubes were placed on the shaker for 2 hrs.

1 hour prior to addition of M. lysodeikticus cell suspension, 5 mL potassium phosphate buffer was added to 1-hour control and coupon comprising a lysozyme tubes, as well as one tube comprising 41 μg lysozyme solution (positive control) and one tube comprising 5 mL of the M. lysodeikticus cell suspension (negative control). These tubes were placed on the shaker for one hour.

The paint coupons were then transferred from each tube to a second reaction tube. 5 mL of the M. lysodeikticus cell suspension was added to both film and KPO₄ buffer incubation buffer. The tubes were placed on the rotating shaker horizontally and shaken for approximately 4 hours, at which time each tube was measured in a Klett-Summerson Photoelectric Colorimeter to determine opacity.

TABLE 19
Assessment of lysis and enzyme leaching (free film) after 1, 2 and 24 hr,
relative to the internal control (i.e., the no lysozyme films).

| | | Replicate 1 | | Replicate 2 | | Replicate 3 | | Average | |
|---|---|---|---|---|---|---|---|---|---|
| | | | Cell | | Cell | | Cell | | Cell |
| | Time | | lysis | | lysis | | lysis | | Lysis |
| Formulation | (hrs) | KU | (dKU) | KU | (dKU) | KU | (dKU) | KU | (dKU) |
| KPO₄ Buffer | | | | | | | | | |
| Control | 1 hr | 110 | 0% | 90 | 0% | 104 | 0% | 101 | 0% |
| Lysozyme | 1 hr | 62 | 39% | 42 | 59% | 52 | 49% | 52 | 49% |
| Control | 2 hr | 92 | 0% | 102 | 0% | 106 | 0% | 100 | 0% |
| Lysozyme | 2 hr | 74 | 26% | 65 | 35% | 65 | 35% | 68 | 32% |
| Control | 24 hr | 95 | 0% | 95 | 0% | 92 | 0% | 94 | 0% |
| Lysozyme | 24 hr | 80 | 15% | 62 | 34% | 55 | 41% | 66 | 30% |
| Film | | | | | | | | | |
| Control | 1 hr | 64 | 0% | 54 | 0% | 38 | 0% | 52 | 0% |
| Lysozyme | 1 hr | 3 | 94% | 40 | 23% | 4 | 92% | 16 | 81% |
| Control | 2 hr | 63 | 0% | 73 | 0% | 72 | 0% | 69 | 0% |
| Lysozyme | 2 hr | 10 | 86% | 23 | 67% | 45 | 35% | 26 | 54% |
| Control | 24 hr | 65 | 0% | 65 | 0% | 68 | 0% | 66 | 0% |
| Lysozyme | 24 hr | 30 | 55% | 52 | 21% | 52 | 21% | 45 | 32% |

KU = Klett Unit, measure of turbidity at 540 nm

At the three time points assayed, lysozyme leached out of films that comprised a lysozyme. The ability of the films comprising a lysozyme to lyse M. lysodeikticus was inversely related to the time the coupon was submerged. Over the first 2 hrs the films lost approximately 21%±3% of the lytic activity per hour. This loss decreased substantially over the following 22 hrs, with the loss slowing to approximately 3% per hour. After 24 hours of liquid submersion, approximately one-third of the activity of a coupon comprising a lysozyme was retained. Though reduction of activity due to leaching may continue, activity may also be permanently retained in the films. The total percentage lysis by coupon and buffer pairs decreased with increasing leaching time.

### EXAMPLE 4

This Example demonstrates the surface efficacy of paint films comprising a lysozyme in actively lyse M. lysodeikticus in a minimally hydrated environment. Materials, reagents and equipment used are shown in the tables below.

TABLE 20
Materials and Reagents

0.1 M potassium phosphate buffer, pH 6.4
Micrococcus lysodeikticus (Worthington Biochemicals, #8736)
Lysozyme (chicken egg white) (Sigma Product #L 6876,
CAS 12650-88-3)
Sherwin-Williams Acrylic Latex paint
15 mL plastic test tubes

TABLE 21
Equipment

Paint spreader (1-8 mil)
Polypropylene blocks
Lightnin Labmaster Mixer
Rotator shaker
Pipetter and tips
Klett-Sumerson Colorimeter (Filter D35: 540 nm)

The reagents prepared included a Micrococcus cell suspension comprising 9 mg Micrococcus lysodeikticus in 25 mL sodium phosphate buffer, and a lysozyme solution comprising a 5 mg/mL stock solution.

The paint formulations prepared for the assay included a Sherwin-Williams Acrylic Latex Control (no additive), and a Sherwin-Williams Acrylic Latex with 1 mg/mL lysozyme. Each paint was mixed with a glass stirring rod and a paint mixer. Each film was immediately drawn onto polypropylene surfaces with a thickness of 8 mil. Cure time was 72 hrs. Assay materials were generated from the polypropylene surface as a 2 cm² (1×2 cm) free film.

The assay procedure included placing individual coupons into separate Petri dishes. Each set of control coupons and coupons comprising a lysozyme was assayed in triplicate. Two controls were set up for this experiment: a M. lysodeikticus suspension control comprising 90 μL 20 mg/mL M. lysodeikticus cell suspension that was pipetted into a petri dish; and a 1 mg/mL lysozyme control comprising 40.64 μL 1 mg/mL lysozyme solution (an amount approximately equal to the amount of lysozyme in the 2 cm² coupon comprising a lysozyme) that was pipetted into a petri dish. M. lysodeikticus cell suspension was distributed onto the surface of each individual coupon in a minimal volume (90 μL). Petri dishes were kept on a flat surface. After 4 hours, KPO₄ buffer was added to all samples to recover the unlysed portion of the M. lysodeikticus cell suspension. The suspension was removed from each dish with a pipette and placed into individual test tubes. Each suspension was read in the Klett-Summerson Photoelectric Colorimeter, using potassium phosphate buffer as a control.

TABLE 22
Surface Efficacy of Films comprising lysozyme in
a low hydration environment.

| | Replicate 1 | | Replicate 2 | | Replicate 3 | | Average | |
|---|---|---|---|---|---|---|---|---|
| | | Cell | | Cell | | Cell | | Cell |
| Formulation | KU | lysis | KU | lysis | KU | lysis | KU | Lysis |
| Suspension/ | | | | | | | | |
| Solution | | | | | | | | |
| Controls | | | | | | | | |
| M. | 80 | | | | | | | |
| lysodeikticus | | | | | | | | |
| Lysozyme | 10 | | | | | | | |
| S-W Acrylic | | | | | | | | |
| Latex | | | | | | | | |
| Control Films | 75 | 6% | 70 | 13% | 78 | 3% | 74 | 7% |
| Lysozyme | 35 | 56% | 19 | 76% | 31 | 61% | 28 | 65% |
| Films | | | | | | | | |

KU = Klett units, measure of turbidity at 540 nm.

The paint comprising a lysozyme contacted with 0.18 mg of a M. lysodeikticus suspension for 4 hours lysed 65%±10% of the Micrococcus cells, compared to only 7%±5% of cells lysed by the paint controls. This demonstrated that lysozyme can function in the low water (i.e., a minimally hydrated) environment of a coating. It is contemplated that a biological assay including a spray application of an assay organism would also demonstrate biostatic and/or biocidal activity.

### EXAMPLE 5

This Example demonstrates the ability of a chymotrypsin to survive the incorporation process into a coating and demonstrates chymotrypsin activity in a coating environment. A chymotrypsin free film assay was used for determining the activity of chymotrypsin, as measured by ester hydrolysis (esterase) activity of a p-nitrophenyl acetate substrate, in free-films using a plate reader. A functioning vent hood was used for the assay as needed for material handling. A Sherwin-Williams Acrylic Latex paint was used. Equipment and reagents that were used are shown in the tables below.

TABLE 23
Equipment

Plate Reader
2 ml microtubes

TABLE 24
Reagents

α-Chymotrypsin from bovine pancreas, Type II
(Sigma Cat# C4129)
4-Nitrophenyl acetate, MW 181.15 (Sigma Cat# N8130)
Trizma base (Sigma Cat# T1503)

Sample preparation included: 14.5 mM p-nitrophenyl acetate (66 mg/25 ml) in isopropyl alcohol, and 200 mM TRIS; pH 7.1 (adjust to pH 7.1 with HCl).

The paint formulations that were prepared included a Sherwin-Williams Acrylic Latex control (no additive), and a Sherwin-Williams Acrylic Latex comprising 200 mg/mL a-Chymotrypsin. Each paint was mixed with a glass stirring rod and a paint mixer. Each film was immediately drawn onto polypropylene surfaces with a thickness of 8 mil. Cure time was 24 days. Materials for assay were generated from the polypropylene surface as 1 cm², 2 cm² and 3 cm free films.

The plate reader assay comprised: cutting free films into appropriate size pieces; adding 600 μL ddH2O into a 2 ml microtube; then adding 750 μL 200 mM TRIS to each microtube; adding 150 μL of 14.5 mM p-nitrophenyl acetate to each tube; and taking the 0 time sample, then adding the free film to the tube (control sample is free film with no chymotrypsin).

The analysis included: taking out 100 μl and reading the absorbance at 405 nm, at the appropriate time points; and determining the initial rate slope by plotting absorbance vs. time to calculate chymotrypsin activity.

TABLE 25A
Absorbance at 405 nm Chymotrypsin in
Sherwin-Williams Acrylic Latex

| Time | Blank | | | 3 cm × 1 cm Control | | |
|---|---|---|---|---|---|---|
| 0 | 0.0480 | 0.0429 | 0.0446 | 0.0480 | 0.0429 | 0.0446 |
| 15 | 0.0482 | 0.0489 | 0.0479 | 0.0518 | 0.0541 | 0.0541 |
| 30 | 0.0571 | 0.0558 | 0.0555 | 0.0596 | 0.0612 | 0.0609 |
| 45 | 0.0608 | 0.0617 | 0.0617 | 0.0679 | 0.0709 | 0.0690 |
| 60 | 0.0683 | 0.0690 | 0.0679 | 0.0773 | 0.0826 | 0.0781 |
| Slope | 0.0004 | 0.0004 | 0.0004 | 0.0005 | 0.0006 | 0.0005 |

TABLE 25B
Absorbance at 405 nm Chymotrypsin in
Sherwin-Williams Acrylic Latex

| Time | 3 cm × 1 cm Enzyme | | | 2 cm × 1 cm Enzyme | | |
|---|---|---|---|---|---|---|
| 0 | 0.0480 | 0.0429 | 0.0446 | 0.0480 | 0.0429 | 0.0446 |
| 15 | 0.2364 | 0.2356 | 0.2347 | 0.1690 | 0.1801 | 0.1749 |
| 30 | 0.4504 | 0.4375 | 0.4208 | 0.3040 | 0.3149 | 0.3172 |
| 45 | 0.6395 | 0.6267 | 0.6441 | 0.4348 | 0.4579 | 0.4474 |
| 60 | 0.8358 | 0.7957 | 0.7970 | 0.5682 | 0.5942 | 0.5930 |
| Slope | 0.0132 | 0.0126 | 0.0128 | 0.0087 | 0.0092 | 0.0091 |

TABLE 25C
Absorbance at 405 nm Chymotrypsin in
Sherwin-Williams Acrylic Latex

| Time | 1 cm × 1 cm Enzyme | | |
|---|---|---|---|
| 0 | 0.0480 | 0.0429 | 0.0446 |
| 15 | 0.1156 | 0.1155 | 0.1164 |
| 30 | 0.1886 | 0.1932 | 0.1872 |
| 45 | 0.2688 | 0.2745 | 0.2684 |
| 60 | 0.3427 | 0.3479 | 0.3578 |
| Slope | 0.0050 | 0.0051 | 0.0052 |

TABLE 26A
Absorbance Averages Chymotrypsin in
Sherwin-Williams Acrylic Latex

| | Absorbance Average | | | | |
|---|---|---|---|---|---|
| | | | Chymo- | | |
| | | Control | trypsin | Chymotrypsin | Chymotrypsin |
| Time | Blank | 3 cm² | 3 cm² | 2 cm² | 1 cm² |
| 0 | 0.0452 | 0.0452 | 0.0452 | 0.0452 | 0.0452 |
| 15 | 0.0483 | 0.0533 | 0.2356 | 0.1747 | 0.1158 |
| 30 | 0.0561 | 0.0606 | 0.4362 | 0.3120 | 0.1897 |
| 45 | 0.0614 | 0.0693 | 0.6368 | 0.4467 | 0.2706 |
| 60 | 0.0684 | 0.0793 | 0.8095 | 0.5851 | 0.3495 |

TABLE 26B
Absorbance Averages Standard Deviations Chymotrypsin in
Sherwin-Williams Acrylic Latex

| | Absorbance Standard Deviation | | | | |
|---|---|---|---|---|---|
| | | | Chymo- | | |
| | | Control | trypsin | Chymotrypsin | Chymotrypsin |
| Time | Blank | 3 cm² | 3 cm² | 2 cm² | 1 cm² |
| 0 | 0.0026 | 0.0026 | 0.0026 | 0.0026 | 0.0026 |
| 15 | 0.0005 | 0.0013 | 0.0009 | 0.0056 | 0.0005 |
| 30 | 0.0009 | 0.0009 | 0.0148 | 0.0071 | 0.0031 |
| 45 | 0.0005 | 0.0015 | 0.0090 | 0.0116 | 0.0034 |
| 60 | 0.0006 | 0.0029 | 0.0228 | 0.0147 | 0.0077 |

TABLE 27
Absorbance vs. Time Slope

| | Slope | U | | |
|---|---|---|---|---|
| Sample | (A/min) | (umol/min) | U Average | U Deviation |
| Blank | 0.0004 | 0.0776 | 0.09 | 0.01 |
| | 0.0004 | 0.0949 | | |
| | 0.0004 | 0.0881 | | |
| Control 3 cm² | 0.0005 | 0.1090 | 0.12 | 0.02 |
| | 0.0006 | 0.1404 | | |
| | 0.0005 | 0.1195 | | |
| Chymotrypsin 3 cm² | 0.0132 | 2.8876 | 2.82 | 0.06 |
| | 0.0126 | 2.7679 | | |
| | 0.0128 | 2.7935 | | |
| Chymotrypsin 2 cm² | 0.0087 | 1.9062 | 1.97 | 0.06 |
| | 0.0092 | 2.0145 | | |
| | 0.0091 | 1.9983 | | |
| Chymotrypsin 1 cm² | 0.0050 | 1.0837 | 1.11 | 0.03 |
| | 0.0051 | 1.1222 | | |
| | 0.0052 | 1.1359 | | |

A chymotrypsin in Sherwin-Williams Acrylic Latex was able to hydrolyze the model substrate at rate 20× faster than the control. The test coupons demonstrate a dose response which corresponds to a hydrolytic capacity of 0.86 umol/min/cm², as formulated in this demonstration.

Quality control included reading and become familiar with the operating instructions for equipment used in the analysis. Operating instructions and preventive maintenance records were placed near the relevant equipment, and kept in a labeled central binder in the work area. Working solutions which are out of date or prepared incorrectly were disposed of and not used.

Safety procedures and precautions included wearing a full length laboratory coat; and not eating, drinking, smoking, use of tobacco products or application of cosmetics near the procedure. Consumables and disposable items that come in contact with or are used in conjunction with samples disposal were in the proper hazard containers. This includes, but is not limited to, pipette tips, bench-top absorbent paper, diapers, kimwipes, test tubes, etc. Biohazard containers were considered full when their contents reach three-quarters of the way to the top of the bag or box. Bench-top biohazard bags were placed into a large biohazard burn box when full. Biohazard containers were not filled to overflowing. Biohazard bags were disposed of by closing with autoclave tape, and autoclaving immediately. Spills and spatters were immediately cleaned from durable surfaces by applying 70% ethanol (for bacteriological spills) to the spill, followed by wiping or blotting. All equipment used in sample analyses were wiped down on a daily basis or whenever tests were performed. Absorbent pads were placed under samples when necessary. Hands were washed with antibacterial soap before exiting the room, when a test was finished, and before the end of the day. The Material Safety Data Sheet (“MSDS”) applicable to each chemical was read. MSDS documents have been prominently posted in the laboratory. During a fire alarm during laboratory operations, evacuation procedures were followed. Nitrile protective gloves were worn whenever handling organophosphates. All organophosphate waste was disposed of properly.

### EXAMPLE 6

This Example demonstrates the ability of a cellulase to survive the incorporation process into a coating and demonstrates cellulase activity in a coating environment. A Glidden Latex paint was used. A plate reader was used to assay a free-film comprising a cellulase for the enzyme's activity. Equipment and reagents that were used are shown in the table below.

TABLE 28
Equipment and Reagents

Equipment
Plate Reader
Reagents
Sodium Acetate (Sigma Cat# S8625)
4-Nitrophenyl β-D-cellobioside (Sigma Cat# N5759)
Cellulase (TCI Cat# C0057)
Sodium Hydroxide

Sample preparation included: 14.5 mM 4-Nitrophenyl β-D-cellobioside in ddH2O; 50 mM sodium acetate buffer; pH 5.0 (adjust to pH 5.0 with HCl); and 2 N NaOH in ddH2O.

The plate reader assay comprised: placing free films into 2 ml microtubes; add 1.2 ml 50 mM sodium acetate buffer, 0.15 ml 14.5 mM 4-Nitrophenyl β-D-cellobioside and 0.15 ml ddH2O, in the 2 ml microtube; placing tubes on rocker; taking out 100 μl from the tubes into a 96-well plate at desired time points; adding 200 μl of 2 N NaOH and reading the absorbance at 405 nm; and determining the initial rate slope by plotting absorbance vs. time to calculate cellulase activity.

The paint formulations that were prepared included a Sherwin-Williams Acrylic Latex control (no additive), and a Sherwin-Williams Acrylic Latex comprising 100 g/gal, 200 g/gal and 300 g/gal cellulase. Each paint was mixed with a glass stirring rod and a paint mixer. Each film was immediately drawn onto polypropylene surfaces with a thickness of 8 mil. Cure time was 24 hrs. Materials for assay were generated from the polypropylene surface as a 3 cm² free film.

TABLE 29A
Glidden Latex Cellulase Free Films -
Dose Response - pNP Absorbance at 405 nm

| Time | | | | | | | |
|---|---|---|---|---|---|---|---|
| (min) | Blank | Control | | | 100 g/gal | | |
| 0 | 0.0600 | 0.0600 | 0.0600 | 0.0600 | 0.0600 | 0.0600 | 0.0600 |
| 30 | 0.0496 | 0.0588 | 0.0488 | 0.0476 | 0.0744 | 0.0753 | 0.0716 |
| 60 | 0.0496 | 0.0605 | 0.0505 | 0.0532 | 0.0975 | 0.1158 | 0.1007 |
| 120 | 0.0507 | 0.0519 | 0.0522 | 0.0514 | 0.1691 | 0.1823 | 0.1672 |
| 180 | 0.0550 | 0.0643 | 0.0583 | 0.0511 | 0.2351 | 0.2312 | 0.2073 |
| 240 | 0.0512 | 0.0614 | 0.0518 | 0.0548 | 0.2876 | 0.2919 | 0.2720 |
| 300 | 0.0491 | 0.0574 | 0.0601 | 0.0575 | 0.3187 | 0.3123 | 0.3083 |
| 360 | 0.0528 | 0.0680 | 0.0540 | 0.0655 | 0.3322 | 0.3215 | 0.3309 |
| Slope | −0.0001 | −0.0001 | 0.0000 | 0.0000 | 0.0009 | 0.0011 | 0.0009 |
| (A/ | | | | | | | |
| min) | | | | | | | |

TABLE 29B
Glidden Latex Cellulase Free Films -
Dose Response - pNP Absorbance at 405 nm

| Time (min) | 200 g/gal | | | 300 g/gal | | |
|---|---|---|---|---|---|---|
| 0 | 0.0600 | 0.0600 | 0.0600 | 0.0600 | 0.0600 | 0.0600 |
| 30 | 0.0986 | 0.0866 | 0.0927 | 0.1207 | 0.1170 | 0.1146 |
| 60 | 0.1387 | 0.1341 | 0.1432 | 0.1637 | 0.1711 | 0.1670 |
| 120 | 0.2285 | 0.2219 | 0.2364 | 0.2864 | 0.2685 | 0.2965 |
| 180 | 0.2891 | 0.2740 | 0.3071 | 0.3304 | 0.3262 | 0.3833 |
| 240 | 0.3174 | 0.3281 | 0.3270 | 0.3543 | 0.3638 | 0.4118 |
| 300 | 0.3449 | 0.3467 | 0.3511 | 0.3759 | 0.3891 | 0.4051 |
| 360 | 0.3714 | 0.3588 | 0.3632 | 0.3808 | 0.3964 | 0.3651 |
| Slope (A/min) | 0.0014 | 0.0014 | 0.0015 | 0.0019 | 0.0017 | 0.0020 |

TABLE 30A
Glidden Latex Cellulase Free Films - Dose
Response - pNP Absorbance at 405 nm Averages

| | Average | | | | |
|---|---|---|---|---|---|
| Time (min) | Blank | Control | 100 g/gal | 200 g/gal | 300 g/gal |
| 0 | 0.0600 | 0.0600 | 0.0600 | 0.0600 | 0.0600 |
| 30 | 0.0496 | 0.0517 | 0.0738 | 0.0926 | 0.1189 |
| 60 | 0.0496 | 0.0547 | 0.1047 | 0.1387 | 0.1674 |
| 120 | 0.0507 | 0.0518 | 0.1729 | 0.2289 | 0.2775 |
| 180 | 0.0550 | 0.0579 | 0.2245 | 0.2901 | 0.3283 |
| 240 | 0.0512 | 0.0560 | 0.2838 | 0.3242 | 0.3591 |
| 300 | 0.0491 | 0.0583 | 0.3131 | 0.3476 | 0.3825 |
| 360 | 0.0528 | 0.0625 | 0.3282 | 0.3645 | 0.3886 |

TABLE 30B
Glidden Latex Cellulase Free Films - Dose
Response - pNP Absorbance at 405 nm Averages' Deviations

Deviation

| Time (min) | Control | 100 g/gal | 200 g/gal | 300 g/gal |
|---|---|---|---|---|
| 0 | 0.0000 | 0.0000 | 0.0000 | 0.0000 |
| 30 | 0.0061 | 0.0019 | 0.0060 | 0.0026 |
| 60 | 0.0052 | 0.0098 | 0.0046 | 0.0052 |
| 120 | 0.0004 | 0.0082 | 0.0073 | 0.0127 |
| 180 | 0.0066 | 0.0151 | 0.0166 | 0.0030 |
| 240 | 0.0049 | 0.0105 | 0.0059 | 0.0067 |
| 300 | 0.0015 | 0.0052 | 0.0032 | 0.0093 |
| 360 | 0.0075 | 0.0058 | 0.0064 | 0.0110 |

A cellulase in a Glidden Latex was able to hydrolyze the model substrate at a rate approximately 100× faster than the control. Quality control and safety procedures were as described in Example 5.

### EXAMPLE 7

This Example demonstrates preparation of technical papers coated with a latex coating comprising an antimicrobial enzyme additive, an antimicrobial peptide additive, or a combination thereof. The additives may be embedded in the coating. The antimicrobial enzyme additive comprised lysozyme, and the antimicrobial peptide additive comprised ProteCoat® (Reactive Surfaces, Ltd.; also described in U.S. patent application Ser. Nos. 10/884,355; 11/368,086; and 11/865,514, each incorporated by reference). Materials that were used are shown in the tables below.

TABLE 31
Materials

30 mM Potassium Phosphate Buffer, was prepared by weighing out 416 mg of potassium
phosphate into 2 × 50 mL conical tubes, and adding 50 mL of water to each tube.
Micrococcus Iysodeikticus (Worthington Biochemicals, #8736), was prepared by weighing out
18 mg of Micrococcus into a single 50 mL conical tube, adding KP0₄ buffer to 50 mLs, and
mixing by inversion.
Lysozyme from chicken egg white (Sigma Product #L 6876; CAS no. 12650-88-3), was
prepared by weighing out 1 g, 0.5 g and 0.1 g lysozyme into 3 × 2 mL eppendorf tubes.
Dilute Acetic Acid Solution was prepared by measuring 1 mL of glacial acetic acid into 11 mLs
of water into a 15 mL conical tube, and adding 50 μl of the dilute acetic acid to 1 mL of water.
ProteCoat ® was used at 125 mg ProteCoat ® per g coating, dispensed as 250 mg ProteCoat ®,
and resuspended in 2 mL dilute acetic acid solution as needed.
5 × 15 mL conical tubes, glass stir rod
P1000 and P200 Pipetteman and Tips
5 × 15 mL conical tubes

Paint formulations comprising enzyme were prepared as follows: 1g lysozyme per 100 g coating; 0.5 g lysozyme per 100 g coating; 0.19 lysozyme per 100 g coating; and a negative control (no additive). Paint formulations comprising a peptide additive were prepared as follows: 125 mg ProteCoat® per 1g coating; 250 mg ProteCoat® per 1g coating; 375 mg ProteCoat® per 1g coating; or a negative control (no additive). Paint formulations comprising peptide and lysozyme were prepared as follows: 375 mg ProteCoat® per 1 g lysozyme (1g) coating; 250 mg ProteCoat® per 1 g lysozyme (0.5 g) coating; 375 mg ProteCoat® per 1 g lysozyme (0.1 g) coating, and a negative Control (no additive). All paint formulations were mixed well. The paper was cut into quarters, coatings drawn onto paper surfaces with a spreader, and wet weight determined. The coated paper was dried at about 37.8° C. for approximately 10 min, and dry weight determined.

A single coating material and one paper stock was evaluated. The paper comprised celluosic fibers typically used in technical paper applications, and had an acrylic latex coating added to the fibers.

TABLE 32
Coating dry components added to paper

| Ingredient | % Dry Weight |
|---|---|
| Kaolin Clay (filler/pigment) | About 0.000000001% to about 90% |
| Titanium Dioxide (pigment) | About 0.000000001% to about 90% |
| Calcium Carbonate (filler/pigment) | About 0.000000001% to about 90% |
| Acrylic Latex (Binder) | About 0.000000001% to about 80% |

To prepare the antimicrobial paper (“AM-Paper”), the antimicrobial additives were formulated for each coating on percentage dry weight to standardize the coating for comparison. The antimicrobial additives are listed in the table below.

TABLE 33
Formulation details for antimicrobial papers

| | | | Final | |
|---|---|---|---|---|
| | | Additive | Dry Weight | |
| Antimicrobial | Designation | Formulation | (gsm) | Additive (%) |
| Control | | | 17.6 | None |
| | | | 21 | None |
| Enzymatic | | | | |
| | A | Powder | 21.9 | 0.2% |
| | B | Powder | 19.4 | 1% |
| | C | Powder | 23.2 | 2% |
| | D | Suspension | 23 | 0.2% |
| | E | Suspension | 23 | 1% |
| | F | Suspension | 20.7 | 2% |
| ProteCoat ® | | | | |
| | G | Suspension | 18.6 | 1% |
| | H | Powder | 23.9 | 2.5% |
| | I | Suspension | 20.6 | 0.5% |
| | J | Powder | 20.9 | 1.25% |
| | K | Powder | 20.9 | 0.25% |
| | L | Powder | 20.7 | 0.75% |
| Enzyme + | | | | |
| ProteCoat ® | | | | |
| | | Powder | 22.5 | 2% + 0.5% |
| | | Powder | 21.9 | 1% + 0.25% |

The antimicrobial additives were weighed out, added to pre-weighed coating suspensions and mixed by hand for 10 to 20 minutes. After mixing, the coating was applied by draw down, in which approximately 3-5 mLs of coating was applied along one 8.5″ edge of an 8.5″×11″ pre-weighed paper, and then spread evenly over the surface of the paper with a calibrated rod by drawing the rod down the full length of the paper. The coated paper was then placed into a 100C oven for 10 to 15 minutes to dry. After drying, the coated paper was weighed to determine the amount of coating on each sheet.

To conduct an assay to qualitatively assess antimicrobial activity, a paper strip of approximately 1 cm×5 cm was cut from the control and each antimicrobial paper. 5 mL of the M. Iysodeikticus suspension was poured into each of 4×15 mL conical tubes. The prepared strip was dropped into the suspension, and mixed occasionally by inversion. Clearing changes were observed.

### EXAMPLE 8

This Example demonstrates and provides a standard spectrophotometric assay procedure for lysozyme activity in a plate reader. Equipment and reagents that were used are shown in the table below.

TABLE 34
Equipment and Reagents

Equipment
Thermo Multiskan Ascent Plate Reader
96-well assay plates
Multi-channels and single-channel pipettes and tips
Reagents
Tris(hydroxymethyl)aminomethane hydrochloride (Tris-HCI): [Sigma, cat # T3253, Molecular
Formula: NH₂C(CH₂OH)₃•HCI, Molecular Weight: 157.60, CAS Number 1185-53-1, pKa
(25° C.) 8.1]
Micrococcus Iysodeikticus cell (Worthington Biochemicals, cat #8736)
Lysozyme: chicken egg white, Sigma cat #L6876; 50,000 U/mg; CAS 12650-88-3; molecular
weight: 14.3 kD; solubility (H₂O) 10 mg/mL; stability-1 month at 2-8° C. Standard: 25 μI of a
500,000 units (10 mg)/mL (10 mM Tris-HCI) will typically lyse E. coli from >1 mL of culture
media cell pellet resuspended in 350 μl buffer (10 mM Tris HCI, pH 8.0, with 0.1 M NaCI, 1 mM
EDTA, and 5% [w/v] Triton X-100). Typical incubation conditions for lysis are 30 min at
37° C.

Micrococcus Iysodeikticus cell suspension was made by adding 9 mg Micrococcus Iysodeikticus to 25 mL 10 mM Tris-HCl, pH 8.0 and mixing well. Lysozyme solution was prepared by adding 10 mg lysozyme in 1 mL 10 mM Tris-HCl, pH 8.0, and mixing well. Reaction buffer was 10 mM Tris-HCl, pH 8.0, with an alternative reaction buffer being 0.1 M KP0₄ pH 6.4.

A standard curve of the M. Iysodeikticus was prepared. The lysozyme stock solution was diluted with the reaction buffer to create the following series: 10 mg/mL (undiluted); 5.0 mg/mL; 2.5 mg/mL; 1 mg/mL; 0.5 mg/mL; 0.1 mg/mL; 0.05 mg/mL; 0.01 mg/mL; 0.005 mg/mL; 0.001 mg/mL; 0.0005 mg/mL; 0.0001 mg/mL, and 0 mg/mL. The controls included 3 replicates of 194 μL M. Iysodeikticus cell suspension plus 6 μL buffer; and 3 replicates of 200 μL buffer.

Analysis of samples included determining activity by monitoring the clearing of the cell suspension at 570 nm and determining the best fit to a standard curve. For a 200 μL assay, 180 μL M. Iysodeikticus in reaction buffer was added to each well 1 to 12 of 3 rows. The reaction was started by adding 20 μL of each lysozyme dilution to each well in the triplicate series. The plate was immediately placed into the reader, and the changes in absorbance at 570 nm (OD₅₇₀) recorded. The number of reads may be 10-20 with second intervals. The plate reader's velocity table contained data for reaction rate in mOD/min. This assay can be scaled by increasing each suspension proportionately (e.g., a 2 mL reaction is used for material strip analysis).

Analysis of the data included calculating the initial velocities for the recorded slopes: [mOD₅₄₀/min]/[slope standard curve (mOD/mg M. Iysodeikticus]/[Iysozyme].

TABLE 35
Assay Standardization

| | |
|---|---|
| Coupon Size | None |
| Test Organism | Micrococcus lysodeikticus |
| Contamination level | 2.5 × 10⁸ cells/mL |
| Assay Time | 4 hr |

TABLE 36
Standardization of Assay

| [Lysozyme], (μg/mL)^{a} | OD₅₇₀ | % Lysis |
|---|---|---|
| 0 | 0.3 | 0.00 |
| 0.78 | 0.26 | 13.33 |
| 1.56 | 0.07 | 76.67 |
| 3.13 | 0.02 | 93.33 |
| 6.25 | 0.005 | 98.33 |
| 12.5 | 0.005 | 98.33 |
| 25 | 0.011 | 96.33 |
| 50 | 0.065 | 78.33 |

^{a}μg/mL = ppm

The M. lysodeikticus assay as described can detect lytic activity down to the fractional to low ppm range. The rate of lysis, in suspension, is 32% (about 8.0×10⁷ cells) of the M. lysodeikticus suspension per μg lysozyme.

### EXAMPLE 9

This Example demonstrates a spectrophotometric assay for antimicrobial paper with a lytic additive. Lysozyme was used as the lytic additive. Equipment and reagents that were used are shown in the table below.

TABLE 37
Equipment and Reagents

Equipment
Spectrophotometer (Thermo Multiskan Ascent Plate Reader)
Cuvettes (96-well assay plates)
Multi-channels and single-channel pipettes and tips
Reagents
Tris(hydroxymethyl)aminomethane hydrochloride (Tris-HCI): [Sigma, cat # T3253,
Molecular Formula: NH₂C(CH₂OH)₃•HCI, Molecular Weight: 157.60, CAS Number 1185-53-
1, pKa (25° C.) 8.1]
Micrococcus Iysodeikticus cell (Worthington Biochemicals, cat #8736)
Lysozyme: chicken egg white, Sigma cat #L6876; 50,000 U/mg; CAS 12650-88-3;
molecular weight: 14.3 kD; solubility (H₂O) 10 mg/mL; stability-1 month at 2-8° C. Standard:
25 μI of a 500,000 units (10 mg)/mL (10 mM Tris-HCI) will typically lyse E. coli from >1 mL
of culture media cell pellet resuspended in 350 μl buffer (10 mM Tris HCI, pH 8.0, with 0.1 M
NaCI, 1 mM EDTA, and 5% [w/v] Triton X-100). Typical incubation conditions for lysis are 30 min
at 37° C.

Micrococcus Iysodeikticus cell suspension was made by adding 9 mg M. Iysodeikticus to 25 mL 10 mM Tris-HCl, pH 8.0 and mixing well. Lysozyme solution was prepared by adding 10 mg lysozyme in 1 mL 10 mM Tris-HCl, pH 8.0, and mixing well. Reaction buffer was 10 mM Tris-HCl, pH 8.0, with an alternative reaction buffer being 0.1 M KP0₄ pH 6.4. Antimicrobial paper coated with a coating comprising lysozyme and control paper was prepared in accordance with Example 7.

A standard curve of the M. Iysodeikticus was prepared. The lysozyme stock solution was diluted with the reaction buffer to create the following series: 10 mg/mL (undiluted); 5.0 mg/mL; 2.5 mg/mL; 1 mg/mL; 0.5 mg/mL; 0.1 mg/mL; 0.05 mg/mL; 0.01 mg/mL; 0.005 mg/mL; 0.001 mg/mL; 0.0005 mg/mL; 0.0001 mg/mL and 0 mg/ml. The controls included 3 replicates of 194 μL M. Iysodeikticus cell suspension plus 6 μL buffer; and 3 replicates of 200 μL buffer. Pipet tips used fitted the pipette (e.g., multichannel pipettes). The liquid level was correct in the tips, as air bubbles, etc may alter volume. Quality control and safety procedures were as described in Example 5.

Antimicrobial paper was cut into appropriately sized strips from both the antimicrobial and control paper. For a 5 mL assay in a 15 mL tube, standard sizes included 5×10 mm, 5×20 mm, and 5×40 mm. These strips could be combined to provide a desired step series.

Analysis of samples included determining activity by monitoring the clearing of the cell suspension at OD₅₇₀ and determining the best fit to a standard curve. For a 5 mL assay, M. Iysodeikticus was added in reaction buffer to an OD₆₀₀ of 0.5. The reaction was started with the addition of the stripes. The tubes were immediately placed at 28° C. for a designated time (e.g., 4 hr and 24 hr). The absorbance at 570 nm was recorded.

Analysis of the data included calculating the initial velocities for the recorded slopes: [OD₆₀₀ min]/[slope standard curve (OD/mg M. Iysodeikticus]/[Iysozyme]

### EXAMPLE 10

This Example demonstrates a biological assay for antimicrobial activity of paper strips comprising an antimicrobial enzyme additive against a microorganism. The antimicrobial enzyme additive comprised lysozyme, the microorganism used was vegetative, gram-positive M. Iysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 38
Equipment and Reagents

Equipment:
Petri Plates
Reagents:
Nutrient Yeast Extract (NBY) NBY Soft Agar
Lysozyme: chicken egg white, Sigma cat #L6876; 50,000 U/mg;
CAS 12650-88-3; molecular weight: 14.3 kD; solubility (H₂O)
10 mg/mL; stability-1 month at 2-8° C. Standard: 25 μI of a
500,000 units (10 mg)/mL (10 mM Tris-HCI) will typically lyse E. coli
from >1 mL of culture media cell pellet resuspended in 350 μl buffer
(10 mM Tris HCI, pH 8.0, with 0.1 M NaCI, 1 mM EDTA, and 5%
[w/v] Triton X-100). Typical incubation conditions for lysis are
30 min at 37° C.

Micrococcus Iysodeikticus cell suspension was made by adding 9 mg Micrococcus Iysodeikticus to NBY and mixing well, with OD₆₀₀ about 0.5. Antimicrobial paper coated with a latex coating comprising lysozyme and control paper was prepared in accordance with Example 7.

The assay include cutting appropriated sized strips of both antimicrobial and control papers (e.g., a. 10×10 mm, 20×20 mm, 40×40 mm, or 50×50 mm). 100 L of the prepared M. Iysodeikticus suspension was transferred to 15 mL tube containing 5 mL NBY Soft Agar, held molten at 55° C., and mixed well. Pipet tips used fitted the pipette (e.g., multichannel pipettes). The liquid level was correct in the tips, as air bubbles, etc may alter volume. The mixture was immediately poured over a prepared sterile agar plate, rotating the dish to completely cover the agar with the M. Iysodeikticus overlay. The dish was covered and allowed to solidify on level surface. The prepared antimicrobial paper(s) were placed (face down) on the soft agar overlay. Coupon(s) up to 20×20 mm were able to be paired with a control on a single petri dish. The dishes were left at 28° C. overnight, and visually evaluated for a zone of clearance around the antimicrobial coupon(s) relative to the control. Quality control and safety procedures were as described in Example 5.

### EXAMPLE 11

This Example demonstrates a biological assay for the antimicrobial activity of a paper strip comprising ProteCoat® against fungal spores. The assay was adapted from ASTM 02020-92, Method A, Standard Test for Mildew (Fungus) Resistance of Paper and Paperboard (Reapproved 2003). Equipment and reagents that were used are shown in the table below.

TABLE 39
Equipment and reagents

Equipment:
Petri Plates
Incubator
Autoclave
Preval Sprayer
Reagents:
Nutrient Yeast Extract (NBY)
NBY Soft Agar
Micrococcus Iysodeikticus cell (Worthington Biochemicals, cat #8736)
ProteCoat ® was used at 125 mg ProteCoat ® per g coating,
dispensed as 250 mg ProteCoat ®, and resuspended in 2 mL
dilute acetic acid solution as needed.

Fusarium oxysporium spores were prepared by maintaining cultures of Fusarium oxysporum f. sp. lycoperici race 1 (RM-1)[FOLRM-1 on Potato Dextrose Agar (PDA) slants. Microconidia of the Fusarium oxysporum f. sp. lycoperici, were obtained by isolating a small portion of an actively growing culture from a PDA plate and transferring to 50 ml a mineral salts medium FLC (Esposito and Fletcher, 1961). The culture was incubated with shaking (125 rpm) at 25° C. After 960 h the fungal slurry consisting of mycelia and microconidia were strained twice through eight layers of sterile cheese cloth to obtain a microconidial suspension. The microcondial suspension was then calibrated with a hemacytometer. All fungal inocula were tested for the absence of contaminating bacteria before their use in experiments. Antimicrobial paper coated with a latex coating comprising ProteCoat® and control paper was prepared in accordance with Example 7.

The assay procedure included: cutting appropriated sized strips of both antimicrobial and control papers (e.g., 40×40 mm or 50×50 mm); centering the strips on a sterile Potato Dextrose Agar plate, treated side up; diluting spores to 2×10³ per mL Potato Dextrose broth; transferring to a calibrated preval sprayer (i.e. dispense 50 μL per single pump action); dispersing spores in a hood onto the agar and paper surface with a single pump action (delivers approximately 100 spores to the area); covering and leaving at ambient conditions; and observing growth over several days, though time of assay will depend on organism. Pipet tips fitted the pipette (e.g., multichannel pipettes). The liquid level was correct in the tips, as air bubbles, etc may alter volume. Quality control and safety procedures were as described in Example 5.

### EXAMPLE 12

This Example demonstrates a paper coating comprising an antimicrobial enzyme additive. The antimicrobial enzyme comprised a lysozyme. Assay standardization and data are shown in the following tables.

TABLE 40
Assay Enzymatic Additive - Lysozyme

| | |
|---|---|
| Example Techniques Used | Example 7 and 9 |
| Coupon Size | Variable, 200-600 mm² |
| Paper Age | 3 months |
| Test Organism | Micrococcus lysodeikticus |
| Contamination level | 2.5 × 10⁸ cells/mL |
| Assay Time | 4 and 24 hrs |

TABLE 41A
Test Strips and Data

| Paper | | | |
|---|---|---|---|
| Type | Paper coupon (mm × mm) | Area (mm²) | [lysozyme], μg |
| 0 | | 0 | |
| 0.2% | 5 × 40 | 200 | 8.76 |
| 1.0% | 5 × 40 | 200 | 38.80 |
| 2.0% | 5 × 40 | 200 | 92.80 |
| 2.0% | 5 × 40 + 5 × 10 | 250 | 116.00 |
| 2.0% | 5 × 40 + 5 × 20 | 300 | 139.20 |
| 2.0% | 5 × 40 + 5 × 40 | 400 | 185.00 |
| 2.0% | 5 × 40 + 5 × 40 + 5 × 10 | 450 | 208.80 |
| 2.0% | 5 × 40 + 5 × 40 + 5 × 20 | 500 | 232.00 |
| 2.0% | 5 × 40 + 5 × 40 + 5 × 40 | 600 | 278.40 |

TABLE 41B
Antimicrobial Strips and Data

| Paper | Paper coupon | 4 hrs | | 24 hrs | |
|---|---|---|---|---|---|
| Type | (mm × mm) | OD₅₇₀ | % Lysis | OD₅₇₀ | % Lysis |
| 0 | | 0.305 | 0.00 | 0.27 | 0.00 |
| 0.2% | 5 × 40 | 0.301 | 1.31 | 0.275 | −1.85 |
| 1.0% | 5 × 40 | 0.277 | 9.18 | 0.2 | 25.93 |
| 2.0% | 5 × 40 | 0.172 | 43.61 | 0.0015 | 99.44 |
| 2.0% | 5 × 40 + 5 × 10 | 0.099 | 67.54 | 0.001 | 99.63 |
| 2.0% | 5 × 40 + 5 × 20 | 0.136 | 55.41 | 0.0025 | 99.07 |
| 2.0% | 5 × 40 + 5 × 40 | 0.017 | 94.43 | 0.005 | 99.81 |
| 2.0% | 5 × 40 + 5 × 40 + 5 × 10 | 0.023 | 92.46 | 0.001 | 99.63 |
| 2.0% | 5 × 40 + 5 × 40 + 5 × 20 | 0.024 | 92.13 | 0.001 | 99.63 |
| 2.0% | 5 × 40 + 5 × 40 + 5 × 40 | 0.015 | 95.08 | 0.0015 | 99.44 |

The rate of lysis upon contact with a coupon cut from antimicrobial treated paper, is approximately 0.5% (1.35×10⁷ cells) per μg lysozyme. This corresponds to a reduction in activity, per μg of lysozyme, of approximately 65% over that observed in suspension. Treated papers of identical size with antimicrobial loadings of 0.2%, 1.0% and 2.0%, demonstrated antimicrobial function. The antimicrobial concentration on a per unit of area for those loadings, is provided in the following table.

TABLE 42
Antimicrobial concentration per unit area

| | | | Lysozyme | | |
|---|---|---|---|---|---|
| Paper | Coating (gsm) | % lysozyme | g/m² | μg/m² | μg/mm² |
| A | 21.9 | 0.2% | 0.0438 | 4.38 × 10⁻⁸ | 0.0438 |
| B | 19.4 | 1.0% | 0.194 | 1.94 × 10⁻⁷ | 0.194 |
| C | 23.2 | 2.0% | 0.464 | 4.64 × 10⁻⁷ | 0.464 |

### EXAMPLE 13

This Example qualitatively demonstrates an antimicrobial enzyme additive combined with an antimicrobial peptide additive to provide antimicrobial functionality to a paper coating formulation. An adaptation of ASTM 02020-92 was used as the assay to demonstrate the growth of a microorganism in a petri dish was inhibited by contact with the treated paper. The antimicrobial enzyme additive comprised lysozyme, and the antimicrobial peptide additive comprised ProteCoat® Reactive Surfaces, Ltd.; also described in U.S. patent application Ser. Nos. 10/884,355; 11/368,086; and 11/865,514, each incorporated by reference).

The spectrophotometric lysozyme assay uses Micrococcus lysodeikticus bacterial cells as a substrate, and measures the change in the turbidity of the cell suspension as described in Example 8 and Example 9. The efficacy of an antimicrobial peptide (e.g., ProteCoat™) may be monitored biologically. Though the contemplated mechanism of action for an antimicrobial or antifouling peptide is similar, i.e. disruption of the structural components of the microbial cell, the cell wall may remain relatively intact. As an antifungal or antimicrobial peptide's biocidal or biostatic activity inhibits the cell, the cell may not lyse for detection of a change in turbidity. Biological assay conditions are shown in the table below.

TABLE 43
Enzymatic Additive - Lysozyme (Qualitative)

| | |
|---|---|
| Example Techniques Used | Example 10 |
| Coupon Size | 100 mm² |
| Paper Age | 3 months |
| Test Organism | Micrococcus lysodeikticus |
| Growth Conditions | 28° C. |

A zone of clearing was seen around the antimicrobial paper in contact with a petri dish covered by M. lysodeikticus, whereas the control paper had no such zone. The coupon of paper was about half the size of the smallest coupons in the quantitative M. lysodeikticus assay, yet growth inhibition was seen.

Assay conditions for Fusarium oxysporum is shown at the table below.

TABLE 44
Enzymatic Additive - ProteCoat ® (Qualitative)

| | |
|---|---|
| Example Techniques Used | Example 11 |
| Coupon Size | 40 × 40 mm |
| Paper Age | 3 months |
| Test Organism | Fusarium oxysporum |
| Contamination level | 100 spore, aerosol delivery |
| Growth Conditions | Ambient |

Overgrowth of both test and control ProteCoat® paper by the fungus, Fusarium oxysporium, was observed. The developmental state of the mycelium on the antimicrobial paper was retarded over that seen in the control paper, indicative of biostatic, and possibly biocide activity.

### EXAMPLE 14

This Example demonstrates synergism between an antimicrobial enzyme additive combined with an antimicrobial peptide additive in a coating applied to papers, and to demonstrate antimicrobial activity of a paper comprising the antimicrobial peptide. The antimicrobial enzyme additive comprised lysozyme, and the antimicrobial peptide additive comprised ProteCoat® (Reactive Surfaces, Ltd.; also described in U.S. patent application Ser. Nos. 10/884,355; 11/368,086; and 11/865,514, each incorporated by reference). Assay conditions are shown at the tables below.

TABLE 45
Enzymatic Additive -
2% Lysozyme + 0.5% ProteCoat ® (Titration Assay)

| | |
|---|---|
| Example Techniques Used | Example 9 |
| Coupon Size | Variable, 0-400 mm² |
| Paper Age | 3 months |
| Test Organism | Micrococcus lysodeikticus |
| Contamination level | 2.5 × 10⁸ cells/mL |
| Assay Time | 3 and 20 hrs |

TABLE 46A
Activity in Treated Papers

| | | | Lysozyme | | ProteCoat ® | |
|---|---|---|---|---|---|---|
| | Strips | Area | | μg/ | | μg/ |
| Paper | (mm × mm) | (mm²) | mg | mL | mg | mL |
| 2% | | 0 | 0 | | | |
| Lysozyme | 5 × 5 | 25 | 11.60 | 2.90 | 0.00 | 0.00 |
| | 5 × 10 | 50 | 23.20 | 5.80 | 0.00 | 0.00 |
| | 5 × 20 | 100 | 46.40 | 11.60 | 0.00 | 0.00 |
| | 5 × 40 | 200 | 92.80 | 23.20 | 0.00 | 0.00 |
| | 5 × 40 + 5 × 5 | 225 | 104.40 | 26.10 | 0.00 | 0.00 |
| | 5 × 40 + 5 × 10 | 250 | 116.00 | 29.00 | 0.00 | 0.00 |
| | 5 × 40 + 5 × 20 | 300 | 139.20 | 34.80 | 0.00 | 0.00 |
| | 5 × 40 + 5 × 40 | 400 | 185.60 | 46.40 | 0.00 | 0.00 |
| 2% | | 0 | | | | |
| Lysozyme + | 5 × 5 | 25 | 11.60 | 2.90 | 2.90 | 0.73 |
| 0.5% | 5 × 10 | 50 | 23.20 | 5.80 | 5.80 | 1.45 |
| ProteCoat ® | 5 × 20 | 100 | 46.40 | 11.60 | 11.60 | 2.90 |
| | 5 × 40 | 200 | 92.80 | 23.20 | 23.20 | 5.80 |
| | 5 × 40 + 5 × 5 | 225 | 104.40 | 26.10 | 26.10 | 6.53 |
| | 5 × 40 + 5 × 10 | 250 | 116.00 | 29.00 | 29.00 | 7.25 |
| | 5 × 40 + 5 × 20 | 300 | 139.20 | 34.80 | 34.80 | 8.70 |
| | 5 × 40 + 5 × 40 | 400 | 185.60 | 46.40 | 46.40 | 11.60 |

TABLE 46B
Activity in Treated Papers

| | | | 3 hrs | | 20 hrs | |
|---|---|---|---|---|---|---|
| | Strips (mm × | Area | | % | | % |
| Paper | mm) | (mm²) | OD₆₀₀ | Lysis | OD₆₀₀ | Lysis |
| 2% | | 0 | 0.266 | 0.00 | 0.258 | 0.00 |
| Lysozyme | 5 × 5 | 25 | 0.259 | 2.63 | 0.25 | 3.10 |
| | 5 × 10 | 50 | 0.259 | 2.63 | 0.23 | 10.85 |
| | 5 × 20 | 100 | 0.256 | 3.76 | 0.145 | 43.80 |
| | 5 × 40 | 200 | 0.228 | 14.29 | 0.038 | 85.27 |
| | 5 × 40 + 5 × 5 | 225 | 0.199 | 25.19 | 0.019 | 92.64 |
| | 5 × 40 + | 250 | 0.148 | 44.36 | 0.011 | 95.74 |
| | 5 × 10 | | | | | |
| | 5 × 40 + | 300 | 0.177 | 33.46 | 0.013 | 94.96 |
| | 5 × 20 | | | | | |
| | 5 × 40 + | 400 | 0.09 | 66.17 | 0.012 | 95.35 |
| | 5 × 40 | | | | | |
| 2% | | 0 | 0.266 | 0.00 | 0.258 | 0.00 |
| Lysozyme + | 5 × 5 | 25 | 0.255 | 4.14 | 0.23 | 10.85 |
| 0.5% | 5 × 10 | 50 | 0.248 | 6.77 | 0.057 | 77.91 |
| ProteCoat ® | 5 × 20 | 100 | 0.237 | 10.90 | 0.016 | 93.80 |
| | 5 × 40 | 200 | 0.195 | 26.69 | 0.012 | 95.35 |
| | 5 × 40 + 5 × 5 | 225 | 0.199 | 25.19 | 0.012 | 95.35 |
| | 5 × 40 + | 250 | 0.15 | 43.61 | 0.012 | 95.35 |
| | 5 × 10 | | | | | |
| | 5 × 40 + | 300 | 0.124 | 53.38 | 0.01 | 96.12 |
| | 5 × 20 | | | | | |
| | 5 × 40 + | 400 | 0.031 | 88.35 | 0.012 | 95.35 |
| | 5 × 40 | | | | | |

The concentration of lysozyme in the papers corresponded to between 2 and 50 ppm, whereas ProteCoat® was between 0.5 and 12 ppm. The comparison of lysis between the 2% lysozyme paper, and the combined paper which contained 2% lysozyme and 0.5% ProteCoat® indicates synergism between the additives. For example, the 100 mm2 coupon size exhibited 44% lysis, whereas the combined paper exhibited 93%. This is an observed/expected (93/44+0) of 2.1, indicative of significant synergism. To further demonstrate this activity, the assay was repeated by titrating the 2% lysozyme paper with individual swaths of 2.5% ProteCoat® paper. 5×10, 5×20, and 5×40 mm² lysozyme paper strips with increasing amount of Protecoat® paper were added to tubes in 4 ml total volume 2.5×10⁸ Micrococcus cells/ml. The assay conditions are shown at the tables below.

TABLE 47
Enzymatic Additive - 2% Lysozyme & 2.5% ProteCoat ® (Titration)

| | |
|---|---|
| Example Techniques Used | Example 9 |
| Coupon Size | Variable |
| Lysozyme | 0-200 mm² |
| ProteCoat ® | 0-200 mm² |
| Paper Age | 3 months |
| Test Organism | Micrococcus lysodeikticus |
| Contamination level | 2.5 × 10⁸ cells/mL |
| Assay Time | 4 and 22 hrs |

TABLE 48
Activity of Protecoat ® paper with 50, 100 and 200 mm² Lysozyme paper
against Micrococcus lysodeikticus

| | | Square | Square area | | |
|---|---|---|---|---|---|
| | Strips | area (mm²) | (mm²) | [lysozyme] | [Protecoat ®] |
| Paper | (mm × mm) | Lysozyme | Protecoat ® | (ug/ml) | (ug/ml) |
| Control | 0 | 0 | 0 | 0 (0) | 0 (0) |
| 2% | 5 × 10 | 50 | 0 | 23.2 (5.8) | 0 (0) |
| Lysozyme | | | | | |
| 2.5% | 5 × 5 | 50 | 25 | 23.2 (5.8) | 15 (3.75) |
| Protecoat ® | 5 × 10 | 50 | 50 | 23.2 (5.8) | 30 (7.5) |
| | 5 × 20 | 50 | 100 | 23.2 (5.8) | 60 (15) |
| | 5 × 40 | 50 | 200 | 23.2 (5.8) | 120 (30) |
| | 5 × 40 × 2 | 50 | 400 | 23.2 (5.8) | 240 (60) |
| Control | 0 | 0 | 0 | 0 (0) | 0 (0) |
| 2% | 5 × 20 | 100 | 0 | 46.4 (11.6) | 0 (0) |
| Lysozyme | | | | | |
| 2.5% | 5 × 5 | 100 | 25 | 46.4 (11.6) | 15 (3.75) |
| Protecoat ® | 5 × 10 | 100 | 50 | 46.4 (11.6) | 30 (7.5) |
| | 5 × 20 | 100 | 100 | 46.4 (11.6) | 60 (15) |
| | 5 × 40 | 100 | 200 | 46.4 (11.6) | 120 (30) |
| | 5 × 40 × 2 | 100 | 400 | 46.4 (11.6) | 240 (60) |
| 2% | 5 × 40 | 200 | 0 | 92.8 (23.2) | 0 (0) |
| Lysozyme | | | | | |
| 2.5% | 5 × 5 | 200 | 25 | 92.8 (23.2) | 15 (3.75) |
| Protecoat ® | 5 × 10 | 200 | 50 | 92.8 (23.2) | 30 (7.5) |
| | 5 × 20 | 200 | 100 | 92.8 (23.2) | 60 (15) |
| | 5 × 40 | 200 | 200 | 92.8 (23.2) | 120 (30) |
| | 5 × 40 × 2 | 200 | 400 | 92.8 (23.2) | 240 (60) |

An example of a calculation for the lysozyme content in 2% lysozyme paper was: 23.2×2% g/m²=0.464 g/m²=0.464 μg/mm². An example of a calculation for the Protecoat® content in 2.5% Protecoat® paper was: 23.9×2.5% g/m²=0.60 g/m²=0.60 μg/mm².

TABLE 49
Activity of Protecoat ® paper with 50, 100 and 200 mm²
Lysozyme paper against Micrococcus lysodeikticus

| | Strips | 4 hrs | | 23 hrs | |
|---|---|---|---|---|---|
| Paper | (mm × mm) | OD₆₀₀ | % Lysis | OD₆₀₀ | % Lysis |
| Control | 0 | 0.278 | 0 | 0.276 | 0 |
| 2% | 5 × 10 | 0.269 | 3.24 | 0.206 | 25.36 |
| Lysozyme | | | | | |
| 2.5% | 5 × 5 | 0.264 | 5.04 | 0.235 | 14.86 |
| Protecoat ® | 5 × 10 | 0.268 | 3.60 | 0.213 | 22.83 |
| | 5 × 20 | 0.269 | 3.24 | 0.197 | 28.62 |
| | 5 × 40 | 0.266 | 4.32 | 0.172 | 37.68 |
| | 5 × 40 × 2 | 0.24 | 13.67 | 0.027 | 90.22 |
| Control | 0 | 0.254 | 0 | 0.229 | 0 |
| 2% | 5 × 20 | 0.224 | 11.81 | 0.026 | 88.65 |
| Lysozyme | | | | | |
| 2.5% | 5 × 5 | 0.22 | 13.39 | 0.023 | 89.96 |
| Protecoat ® | 5 × 10 | 0.204 | 19.69 | 0.013 | 94.32 |
| | 5 × 20 | 0.212 | 16.54 | 0.019 | 91.70 |
| | 5 × 40 | 0.178 | 29.92 | 0.014 | 93.89 |
| | 5 × 40 × 2 | 0.194 | 23.62 | 0.027 | 88.21 |
| 2% | 5 × 40 | 0.203 | 20.08 | 0.019 | 91.70 |
| Lysozyme | | | | | |
| 2.5% | 5 × 5 | 0.181 | 28.74 | 0.009 | 96.07 |
| Protecoat ® | 5 × 10 | 0.175 | 31.10 | 0.01 | 95.63 |
| | 5 × 20 | 0.165 | 35.04 | 0.012 | 94.76 |
| | 5 × 40 | 0.128 | 49.61 | 0.012 | 94.76 |
| | 5 × 40 × 2 | 0.145 | 42.91 | 0.019 | 91.70 |

TABLE 50A
% Lysis (relative to control without
Protecoat ® added) at given time

| Square Area | 4 hr | | |
|---|---|---|---|
| (mm²) of | 50 mm² | 100 mm² | 200 mm² |
| Protecoat ® | Lysozyme | Lysozyme | Lysozyme |
| paper | paper | paper | paper |
| 0 | 3.24 | 11.81 | 20.08 |
| 25 | 5.04 | 13.39 | 28.74 |
| 50 | 3.60 | 19.69 | 31.10 |
| 100 | 3.24 | 16.54 | 35.04 |
| 200 | 4.32 | 29.92 | 49.61 |
| 400 | 13.67 | 23.62 | 42.91 |

TABLE 50B
% Lysis (relative to control without
Protecoat ® added) at given time

| Square Area | 22 hr | | |
|---|---|---|---|
| (mm²) of | 50 mm² | 100 mm² | 200 mm² |
| Protecoat ® | Lysozyme | Lysozyme | Lysozyme |
| paper | paper | paper | paper |
| 0 | 25.36 | 88.65 | 91.70 |
| 25 | 14.86 | 89.96 | 96.07 |
| 50 | 22.83 | 94.32 | 95.63 |
| 100 | 28.62 | 91.70 | 94.76 |
| 200 | 37.68 | 93.89 | 94.76 |
| 400 | 90.22 | 88.21 | 91.70 |

The assay was repeated by titrating the 2% lysozyme paper with individual swaths of 2.5% ProteCoat® paper. Lysozyme in technical papers added to an assay at concentrations greater than 10 ppm exhibited antimicrobial activity in the M. lysodeikticus assay. Lysozyme at approximately 5 ppm in the assay did not exhibit significant antimicrobial activity over the course of the assay (20 hrs). The addition of ProteCoat® papers, with between 3 and 60 ppm ProteCoat® to the assay significantly enhanced the lytic activity of lysozyme, or possibly the reverse. This was also true with the 5 ppm lysozyme, in which the lytic activity was doubled by the addition of between 3 and 60 ppm ProteCoat® to the assay. The peptide additive may be enhancing the activity of the enzyme, or the enzyme enhancing the activity of the peptide, or both, to produce these results.

### EXAMPLE 15

This Example demonstrates a spectrophotometric assay for an antimicrobial coating with a lytic additive. The lytic additive comprised a lysozyme. The antimicrobial coatings were created using acrylic latex, commercially available paints. Equipment and reagents that were used are shown in the table below.

TABLE 51
Equipment and Reagents

Equipment
Spectrophotometer (Thermo Multiskan Ascent Plate Reader)
Cuvettes (96-well assay plates)
Multi-channels and single-channel pipettes and tips
Reagents
Tris(hydroxymethyl)aminomethane hydrochloride (Tris-HCI): [Sigma,
cat # T3253, Molecular Formula: NH₂C(CH₂OH)₃•HCI,
Molecular Weight: 157.60, CAS Number 1185-53-1, pKa (25° C.) 8.1]
Micrococcus Iysodeikticus cell (Worthington Biochemicals, cat #8736)
Lysozyme: chicken egg white {Sigma cat #L6876; 50,000 U/mg;
CAS 12650-88-3; molecular weight: 14.3 kD; solubility (H₂O)
10 mg/mL; stability - 1 month at 2-8° C. Standard: 25 μI of a 500,000
units (10 mg)/mL (10 mM Tris-HCI) will typically lyse E. coli from
>1 mL of culture media cell pellet resuspended in 350 μl buffer
(10 mM Tris HCI, pH 8.0, with 0.1 M NaCI, 1 mM EDTA, and 5%
[w/v] Triton X-100). Typical incubation conditions for lysis are 30 min
at 37° C.}

A Micrococcus Iysodeikticus cell suspension was made by adding 1.5 mg Micrococcus Iysodeikticus to 1 mL 10 mM Tris pH 8.0 and mixing well. A lysozyme solution was prepared by adding 10 mg lysozyme in 1 mL ddH₂0, and mixing well.

The lysozyme stock solution was mixed into Sherwin Williams Acrylic (SW) or Glidden latex paint (1 part water:7 part paint). 4 mil, 6 mil, and 8 mil free films were created from Sherwin Williams paint comprising a lysozyme, a Glidden paint comprising a lysozyme, and controls for both. The plate controls included 3 replicates of 50 μL M. Iysodeikticus cell suspension plus 50 μL buffer; and 3 replicates of 100 μL buffer. Pipet tips used fitted the pipette (e.g., multichannel pipettes). The liquid level was correct in the tips, as air bubbles, etc may alter volume. Quality control and safety procedures were as described in Example 5.

The antimicrobial films were cut into appropriately sized strips from both the antimicrobial and control coating. For a 5 mL assay in a 15 mL tube, standard size was 1×1 cm.

Analysis of samples included determining activity by monitoring the clearing of the cell suspension at OD₄₀₅ and determining the best fit to a standard curve. The reaction was started with the addition of 5 ml of the M. Iysodeikticus stock. The tubes were immediately placed on a rocker for 3 hr; 100 μl samples were taken at 3 hr, and the absorbance at 405 nm was recorded.

TABLE 52
Sample Lysis Averages and Deviations

| | | |
|---|---|---|
| | Avg. % | |
| | Lysis at | Standard |
| Sample | 3 hr | Deviation |

| | | | |
|---|---|---|---|
| SW Control | 4 mils | 11.1057 | 0.5752 |
| | 6 mils | 12.2932 | 0.3812 |
| | 8 mils | 12.2802 | 0.5752 |
| SW Lysozyme | 4 mils | 65.0651 | 1.3638 |
| | 6 mils | 74.5744 | 3.8272 |
| | 8 mils | 84.2325 | 4.1432 |
| Glidden Control | 4 mils | 4.8514 | 0.4912 |
| | 6 mils | 5.1005 | 0.0569 |
| | 8 mils | 5.1749 | 0.6266 |
| Glidden | 4 mils | 18.3760 | 0.5846 |
| Lysozyme | 6 mils | 23.1840 | 3.6201 |
| | 8 mils | 29.1666 | 1.9095 |

Analysis of the data included calculating the initial velocities for the recorded slopes: [OD₄₀₅ min]/[slope standard curve (OD/mg M. Iysodeikticus]/[Iysozyme],

### EXAMPLE 16

This Example demonstrates a biological assay for antimicrobial activity of coatings comprising an antimicrobial enzyme additive against a microorganism. The antimicrobial enzyme additive comprised lysozyme, the microorganism used comprised vegetative, gram-positive M. Iysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 53
Equipment and Reagents

Equipment:
Petri Plates
Reagents:
Luria Broth Agar (LBA)
Lysozyme: chicken egg white, Sigma cat #L6876; 50,000 U/mg;
CAS 12650-88-3; molecular weight: 14.3 kD; solubility (H₂O) 10 mg/mL;
stability - 1 month at 2-8° C. Standard: 25 μI of a 500,000 units
(10 mg)/mL (10 mM Tris-HCI) will typically lyse E. coli from >1 mL
of culture media cell pellet resuspended in 350 μl buffer (10 mM Tris
HCI, pH 8.0, with 0.1 M NaCI, 1 mM EDTA, and 5% [w/v] Triton
X-100). Typical incubation conditions for lysis are 30 min at 37° C.

A Micrococcus Iysodeikticus cell suspension was made by adding 1.5 mg M. Iysodeikticus to 10 mM Tris, pH 8.0, and mixing well. A lawn of M. Iysodeikticus was generated by spreading 200 μl of this suspension onto an LBA plate, using a glass spreading rod. An antimicrobial latex coating comprising lysozyme and a control film was prepared in accordance with Example 15.

The assay include cutting appropriated sized strips of both antimicrobial and control latex films (e.g., a 1×1 cm). In triplicate the free films are carefully placed onto the surface of the petri dishes spaced out equally. This procedure was repeated for each of the paint film types/thicknesses.

The paint films comprising a lysozyme were active in lysing M. Iysodeikticus, producing circular zones of clearing. The difference in Zone of Clearing Diameter between the different thicknesses of film was deemed negligible.

TABLE 54
Diameter (cm) of Zones of Clearing

| Sample | 4 mils | 6 mils | 8 mils |
|---|---|---|---|
| Glidden Lysozyme | 2.8 | 2.8 | 2.8 |
| | 2.8 | 2.9 | 2.8 |
| | 2.7 | 2.9 | 2.9 |
| Glidden Control | 0 | 0 | 0 |
| | 0 | 0 | 0 |
| | 0 | 0 | 0 |
| Sherwin Williams | 2.1 | 1.9 | 2.2 |
| Lysozyme | 2.1 | 1.9 | 1.9 |
| | 2 | 2 | 1.8 |
| Sherwin Williams | 0 | 0 | 0 |
| Lysozyme | 0 | 0 | 0 |
| | 0 | 0 | 0 |

### EXAMPLE 17

This Example demonstrates a qualitative biological assay for survivability of an antimicrobial latex coating comprising an antimicrobial enzyme additive against a microorganism. The antimicrobial enzyme additive comprised lysozyme, the microorganism used comprised vegetative, gram-positive M. Iysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 55
Equipment and Reagents

Equipment:
Petri Plates
Reagents:
Luria Broth Agar (LBA)
Lysozyme: chicken egg white, Sigma cat #L6876; 50,000 U/mg;
CAS 12650-88-3; molecular weight: 14.3 kD; solubility (H₂O)
10 mg/mL; stability - 1 month at 2-8° C. Standard: 25 μI of a
500,000 units (10 mg)/mL (10 mM Tris-HCI) will typically lyse E. coli
from >1 mL of culture media cell pellet resuspended in 350 μl buffer
(10 mM Tris HCI, pH 8.0, with 0.1 M NaCI, 1 mM EDTA, and 5% [w/v]
Triton X-100). Typical incubation conditions for lysis are 30 min at 37° C.

A Micrococcus Iysodeikticus cell suspension was made by adding 1.5 mg M. Iysodeikticus to 10 mM Tris, pH 8.0, and mixing well. A lawn of M. Iysodeikticus was generated by spreading 200 μl of this suspension onto an LBA plate, using a glass spreading rod.

The paint formulations that were prepared included a Sherwin-Williams Acrylic Latex or a Glidden Acrylic Latex as controls (no additive), and both a Sherwin-Williams Acrylic Latex or a Glidden Acrylic Latex comprising 10 mg/mL Lysozyme (ddH2O). Each paint was made by adding 1 part additive to 7 parts paint, and then mixed with a glass stirring rod and a paint mixer. Each film was immediately drawn onto polypropylene surfaces with a thickness of 4 mil, 6 mil, and 8 mil. Cure time was 24 days. Materials for assay were generated from the polypropylene surface as 1 cm² free films.

The assay include cutting appropriately sized strips of both antimicrobial and control latex films (e.g., a 1×1 cm). In triplicate the free films were carefully placed onto the surface of the petri dishes spaced out equally. This procedure was repeated for each of the paint film types/thicknesses.

After 24 hrs incubation, the diameter of the zones of clearing was measured for each film. Using sterile tweezer, the films were removed and transfer to a new LBA plate spread with M. Iysodeikticus in the same orientation as the plates the films were removed from. Repeat the procedure of measuring the zones of clearing through transfer to a new plate every day for 5 days.

TABLE 56
Average Diameter (cm) of Zones of Clearing

| | | | Standard | | Standard | | |
|---|---|---|---|---|---|---|---|
| | | 4 mils | Deviation | 6 mils | Deviation | 8 mils | Standard Deviation |
| Day 1 | Glidden | N/A | N/A | N/A | N/A | 0 | 0 |
| | Control | | | | | | |
| | Glidden | 2.5667 | 0.0577 | 2.5333 | 0.0577 | 2.7000 | 0.0000 |
| | Lysozyme | | | | | | |
| Day 2 | Glidden | N/A | N/A | N/A | N/A | 0 | 0 |
| | Control | | | | | | |
| | Glidden | 2.0000 | 0.0000 | 2.0000 | 0.0000 | 2.2000 | 0.0000 |
| | Lysozyme | | | | | | |
| Day 3 | Glidden | N/A | N/A | N/A | N/A | 0 | 0 |
| | Control | | | | | | |
| | Glidden | 1.4667 | 0.0577 | 1.6667 | 0.0577 | 1.9000 | 0.0000 |
| | Lysozyme | | | | | | |
| Day 4 | Glidden | N/A | N/A | N/A | N/A | 0 | 0 |
| | Control | | | | | | |
| | Glidden | 1.4333 | 0.1155 | 1.5667 | 0.0577 | 1.8000 | 0.0000 |
| | Lysozyme | | | | | | |
| Day 5 | Glidden | N/A | N/A | N/A | N/A | 0 | 0 |
| | Control | | | | | | |
| | Glidden | 1.2667 | 0.0577 | 1.4500 | 0.0707 | 1.6333 | 0.0577 |
| | Lysozyme | | | | | | |

¹N/A in this chart just means not available/not applicable.

There were no 4 mil or 6 mil controls tested due to a limited LBA plate supply, though 8 mil control films were tested. The standard deviations for the 8 mil controls to 0, because all 3 controls produced a 0 cm zone of clearing in each case.

The paint films comprising lysozyme were active in lysing M. Iysodeikticus, producing circular zones of clearing, for five cycles of contaminant control. The difference in Zone of Clearing Diameter between the different thicknesses of each film appeared negligible.

### EXAMPLE 18

This Example demonstrates a sulfatase's activity in free-films using a plate reader. Equipment and reagents used are shown in the table below.

TABLE 57
Equipment and Reagents

Equipment
Plate Reader
96-well plate
2 ml microtubes
Reagents
Sulfatase from Aerobacter aerogenes (Sigma Cat# S1629-50UN)
Potassium 4-Nitrophenyl sulfate (MW 257.27; Sigma Cat# N3877)
Trizma base (Sigma Cat# T1503)

Samples preparation procedure included preparing: 14.5 mM potassium 4-nitrophenyl sulfate in isopropyl alcohol; and 200 mM TRIS, adjusted to pH 7.1 with HCl.

The paint formulations that were prepared included a Sherwin-Williams Acrylic Latex control (no additive), and a Sherwin-Williams Acrylic Latex comprising sulfatase. 63 enzyme units of sulfatase was admixed with 1 part water, then added to 7 parts paint. Each paint was mixed with a glass stirring rod and a paint mixer. Each film was immediately drawn onto polypropylene surfaces with a thickness of 8 mil. Cure time was 24 hours. Materials for assay were generated from the polypropylene surface as 3 cm² free films.

The plate reader assay included: cutting free films into appropriate size pieces; adding 1350 uL 200 mM TRIS into each microtube; adding 150 uL of 14.5 mM potassium 4-nitrophenyl sulfate to each tube; taking the 0 time sample; then adding the free films to the tubes, with the control sample being free film with no sulfatase. Quality control and safety procedures were as described in Example 5, including use of a hood for material handling as needed.

Analysis included: taking 100 ul at the appropriate time points from each microtube and reading the absorbance at 405 nm; and determining the initial rate slope by plotting absorbance vs. time to calculate sulfatase activity.

TABLE 58A
Absorbance at 405 nm

| Time | Blank | | |
|---|---|---|---|
| 0 | 0.0410 | 0.0408 | 0.0401 |
| 15 | 0.0414 | 0.0409 | 0.0408 |
| 30 | 0.0411 | 0.0400 | 0.0410 |
| 60 | 0.0405 | 0.0410 | 0.0410 |
| 120 | 0.0428 | 0.0409 | 0.0412 |
| Slope | 0.0000 | 0.0000 | 0.0000 |

TABLE 58B
Absorbance at 405 nm

| Time | 3 cm × 1 cm Control | | | 3 cm × 1 cm Enzyme | | |
|---|---|---|---|---|---|---|
| 0 | 0.0410 | 0.0408 | 0.0401 | 0.0410 | 0.0408 | 0.0401 |
| 15 | 0.0420 | 0.0408 | 0.0407 | 0.0595 | 0.0592 | 0.0607 |
| 30 | 0.0450 | 0.0414 | 0.0413 | 0.0800 | 0.0819 | 0.0818 |
| 60 | 0.0421 | 0.0448 | 0.0500 | 0.1243 | 0.1307 | 0.1291 |
| 120 | 0.0415 | 0.0422 | 0.0430 | 0.2024 | 0.2138 | 0.2159 |
| Slope | 0.0000 | 0.0000 | 0.0000 | 0.0014 | 0.0015 | 0.0015 |

TABLE 59A
Average Absorbance at 405 nm

| | Absorbance Average | | |
|---|---|---|---|
| Time | Blank | Control 3 cm² | Sulfatase 3 cm² |
| 0 | 0.0406 | 0.0406 | 0.0406 |
| 15 | 0.0410 | 0.0412 | 0.0598 |
| 30 | 0.0407 | 0.0426 | 0.0812 |
| 60 | 0.0408 | 0.0456 | 0.1280 |
| 120 | 0.0416 | 0.0422 | 0.2107 |

TABLE 59B
Average Absorbance at 405 nm
Standard Deviations

| | Absorbance Standard Deviation | | |
|---|---|---|---|
| Time | Blank | Control 3 cm² | Sulfatase 3 cm² |
| 0 | 0.0005 | 0.0005 | 0.0005 |
| 15 | 0.0003 | 0.0007 | 0.0008 |
| 30 | 0.0006 | 0.0021 | 0.0011 |
| 60 | 0.0003 | 0.0040 | 0.0033 |
| 120 | 0.0010 | 0.0008 | 0.0073 |

TABLE 60
Absorbance vs. Time Slope Activity Data

| | | U | U | U |
|---|---|---|---|---|
| Sample | Slope (A/min) | (umol/min) | Average | Deviation |
| Blank | 0.0000 | 0.0028 | 0.0016 | 0.0012 |
| | 0.0000 | 0.0005 | | |
| | 0.0000 | 0.0015 | | |
| Control 3 cm² | 0.0000 | −0.0009 | 0.0036 | 0.0045 |
| | 0.0000 | 0.0038 | | |
| | 0.0000 | 0.0080 | | |
| Sulfatase 3 cm² | 0.0014 | 0.2971 | 0.3133 | 0.0141 |
| | 0.0015 | 0.3200 | | |
| | 0.0015 | 0.3229 | | |

### EXAMPLE 19

This Example demonstrates a phosphodiesterase I assay using a plate reader. The equipment and reagents used are shown in the table below.

TABLE 61
Equipment and reagents

Equipment
Plate Reader
96-well plate
Reagents
Phosphodiesterase I from Crotalus adamanteus Venom
(Worthington Cat# LS003926)
Thymidine 5-monophosphate p-nitrophenyl ester sodium salt
(MW 465.3; Sigma Cat# T4510)
Trizma base (Sigma Cat# T1503)

Samples prepared included: 14.5 mM Thymidine 5-monophosphate p-nitrophenyl ester sodium salt in ddH₂O; a 124 U/ml ddH₂O enzyme solution; and 200 mM TRIS (adjusted to pH 7.1 with HCl).

The plate reader assay comprised: diluting enzyme solution 1:1 and 1:3; adding 16 ul of each enzyme dilution in triplicate into a 96-well plate, with a control sample prepared by adding 16 ul ddH₂O; adding 24 ul ddH₂O into each well; adding 50 ul 200 mM TRIS to each well; and adding 10 uL of 14.5 mM Thymidine 5-monophosphate p-nitrophenyl ester sodium salt in ddH₂O to each well. Quality control and safety procedures were as described in Example 5, including use of a hood for material handling as needed.

The analysis included: taking 500 readings every 10 seconds at 405 nm; and determining the initial rate slope by plotting absorbance vs. time to calculate phosphodiesterase I activity. Summary results are below.

TABLE 62
Phosphodiesterase Activity

| | Slope | U | | U |
|---|---|---|---|---|
| Sample | (A/min) | (umol/min) | U Average | Deviation |
| 2U | 0.1069 | 23.39 | 20.48 | 2.58 |
| | 0.0895 | 19.60 | | |
| | 0.0844 | 18.47 | | |
| 1U | 0.0764 | 16.73 | 15.27 | 1.69 |
| | 0.0715 | 15.64 | | |
| | 0.0613 | 13.42 | | |

TABLE 63
Phosphodiesterase Activity

| | Slope | U | | U |
|---|---|---|---|---|
| Sample | (A/min) | (umol/min) | U Average | Deviation |
| 0.5U | 0.0508 | 11.12 | 10.62 | 0.54 |
| | 0.0488 | 10.69 | | |
| | 0.0459 | 10.05 | | |
| Control | −0.0002 | −0.04 | −0.04 | 0.03 |
| | −0.0004 | −0.08 | | |
| | −0.0001 | −0.01 | | |

### EXAMPLE 20

This Example demonstrates a phosphodiesterase I activity assay in free-films using a plate reader.

TABLE 64
Equipment and reagents

Equipment
Plate Reader
96-well plate
2 ml microtubes
Reagents
Phosphodiesterase I from Crotalus adamanteus Venom
(Worthington Cat# LS003926)
Thymidine 5-monophosphate p-nitrophenyl ester sodium salt
(MW 465.3; Sigma Cat#
T4510)
Trizma base (Sigma Cat# T1503)

Samples prepared included: 14.5 mM Thymidine 5-monophosphate p-nitrophenyl ester sodium salt in ddH₂O; and 200 mM TRIS (adjusted to pH 7.1 with HCl).

The paint formulations that were prepared included a Sherwin-Williams Acrylic Latex control (no additive), and a Sherwin-Williams Acrylic Latex comprising phosphodiesterase I. 113 enzyme units of phosphodiesterase I was admixed with 1 part water, then added to 7 parts paint. Each paint was mixed with a glass stirring rod and a paint mixer. Each film was immediately drawn onto polypropylene surfaces with a thickness of 8 mil. Cure time was 24 hours. Materials for assay were generated from the polypropylene surface as 1 cm², 2 cm² and 3 cm² free films.

The plate reader assay comprised: cutting free films into appropriate sized pieces and place them into microtubes, though blank samples have no paint film inside the microtube; adding 600 ul ddH₂O into each microtube; adding 750 ul 200 mM TRIS into each microtube; and adding 150 uL of 14.5 mM Thymidine 5-monophosphate p-nitrophenyl ester sodium salt in ddH₂O into each microtube. Quality control and safety procedures were as described in Example 5, including use of a hood for material handling as needed.

Analysis included: taking out 100 ul from each microtube at the appropriate time points, and reading the absorbance at 405 nm; and determining the initial rate slope by plotting absorbance vs. time to calculate phosphodiesterase I activity.

TABLE 65A
Phosphodiesterase I Sample absorbance at 405 nm

| Time (min) | Blank | | | 3 cm × 1 cm Control | | |
|---|---|---|---|---|---|---|
| 0 | 0.0432 | 0.0401 | 0.0438 | 0.0432 | 0.0401 | 0.0438 |
| 30 | 0.0385 | 0.0388 | 0.0384 | 0.0425 | 0.0441 | 0.0409 |
| 60 | 0.0412 | 0.0395 | 0.0391 | 0.0485 | 0.0402 | 0.0431 |
| 120 | 0.0408 | 0.0398 | 0.0394 | 0.0443 | 0.0408 | 0.0410 |
| 240 | 0.0410 | 0.0396 | 0.0442 | 0.0411 | 0.0421 | 0.0411 |
| 1200 | 0.0464 | 0.0411 | 0.0420 | 0.0433 | 0.0418 | 0.0416 |
| Slope | 0.0000 | 0.0000 | 0.0000 | 0.0000 | 0.0000 | 0.0000 |
| (A/min) | | | | | | |

TABLE 65B
Phosphodiesterase I Sample absorbance at 405 nm

| Time (min) | 3 cm × 1 cm Enzyme | | | 2 cm × 1 cm Enzyme | | |
|---|---|---|---|---|---|---|
| 0 | 0.0432 | 0.0401 | 0.0438 | 0.0432 | 0.0401 | 0.0438 |
| 30 | 0.0582 | 0.0567 | 0.0598 | 0.0515 | 0.0486 | 0.0497 |
| 60 | 0.0807 | 0.0787 | 0.0822 | 0.0671 | 0.0628 | 0.0648 |
| 120 | 0.1459 | 0.1348 | 0.1424 | 0.1093 | 0.0997 | 0.1076 |
| 240 | 0.2720 | 0.2534 | 0.2663 | 0.2058 | 0.1854 | 0.1985 |
| 1200 | 0.6818 | 0.6674 | 0.6647 | 0.6234 | 0.5894 | 0.6073 |
| Slope (A/min) | 0.0010 | 0.0009 | 0.0010 | 0.0007 | 0.0006 | 0.0007 |

TABLE 65C
Phosphodiesterase I Sample absorbance at 405 nm

| Time (min) | 1 cm × 1 cm Enzyme | | |
|---|---|---|---|
| 0 | 0.0432 | 0.0401 | 0.0438 |
| 30 | 0.0459 | 0.0451 | 0.0455 |
| 60 | 0.0547 | 0.0509 | 0.0543 |
| 120 | 0.0800 | 0.0714 | 0.0793 |
| 240 | 0.1420 | 0.1151 | 0.1204 |
| 1200 | 0.4900 | 0.4191 | 0.4146 |
| Slope (A/min) | 0.0004 | 0.0003 | 0.0003 |

TABLE 66A
Phosphodiesterase I Sample absorbance Average at 405 nm

| | | | 3 cm² | 2 cm² | 1 cm² |
|---|---|---|---|---|---|
| Time | | 3 cm² | Phospho- | Phospho- | Phospho- |
| (min) | Blank | Control | diesterase I | diesterase I | diesterase I |
| 0 | 0.0424 | 0.0424 | 0.0424 | 0.0424 | 0.0424 |
| 30 | 0.0386 | 0.0425 | 0.0582 | 0.0499 | 0.0455 |
| 60 | 0.0399 | 0.0439 | 0.0805 | 0.0649 | 0.0533 |
| 120 | 0.0400 | 0.0420 | 0.1410 | 0.1055 | 0.0769 |
| 240 | 0.0416 | 0.0414 | 0.2639 | 0.1966 | 0.1258 |

TABLE 66B
Phosphodiesterase I Sample absorbance Deviation at 405 nm

| | | | 3 cm² | 2 cm² | 1 cm² |
|---|---|---|---|---|---|
| Time | | 3 cm² | Phospho- | Phospho- | Phospho- |
| (min) | Blank | Control | diesterase I | diesterase I | diesterase I |
| 0 | 0.0020 | 0.0020 | 0.0020 | 0.0020 | 0.0020 |
| 30 | 0.0002 | 0.0016 | 0.0016 | 0.0015 | 0.0004 |
| 60 | 0.0011 | 0.0042 | 0.0018 | 0.0022 | 0.0021 |
| 120 | 0.0007 | 0.0020 | 0.0057 | 0.0051 | 0.0048 |
| 240 | 0.0024 | 0.0006 | 0.0095 | 0.0103 | 0.0142 |

TABLE 67
Phosphodiesterase I Activity

| | Slope | U | | |
|---|---|---|---|---|
| Sample | (A/min) | (umol/min) | U Average | U Deviation |
| Blank | 0.0000 | −0.0004 | 0.00 | 0.00 |
| | 0.0000 | 0.0001 | | |
| | 0.0000 | 0.0024 | | |
| Control 3 cm² | 0.0000 | −0.0024 | 0.00 | 0.00 |
| | 0.0000 | 0.0005 | | |
| | 0.0000 | −0.0018 | | |
| Phosphodiesterase | 0.0010 | 0.2151 | 0.21 | 0.01 |
| 3 cm² | 0.0009 | 0.1987 | | |
| | 0.0010 | 0.2081 | | |
| Phosphodiesterase | 0.0007 | 0.1530 | 0.15 | 0.01 |
| 2 cm² | 0.0006 | 0.1362 | | |
| | 0.0007 | 0.1468 | | |
| Phosphodiesterase | 0.0004 | 0.0937 | 0.08 | 0.01 |
| 1 cm² | 0.0003 | 0.0703 | | |
| | 0.0003 | 0.0738 | | |

### EXAMPLE 21

This Example demonstrates lipase activity in a Glidden alkyd/oil solvent-borne coating. The materials used are shown in the table below.

TABLE 68
Materials

200 mM TRIS Buffer (Sigma Product # T1503);
brought to pH = 7.1 with HCl
4-nitrophenyl acetate (Sigma Product # N8130);
14.5 mM solution in isopropyl alcohol
Lipase from porcine pancreas (Sigma Product # L3126)
2 mL microtubes
Pipette
Pipette Tips
Plate Reader
96-well Plate

The assay procedure included: cutting appropriate coupon sizes; placing individual coupons into labeled 2 mL microtubes, with each of the coupon sizes are tested in triplicate; adding 750 ul 200 mM TRIS to each microtube; adding 600 ul ddH2O to each microtube; adding 150 ul 14.5 mM p-nitrophenyl acetate to each microtube; preparing control samples (no films) to have 750 ul 200 mM TRIS, 600 ul ddH2O, and 150 ul 14.5 mM p-nitrophenyl acetate; removing at each desired time point, 100 ul and reading the absorbance at 405 nm in a 96-well plate; and plotting absorbance vs. time to calculate the initial rate slope.

TABLE 69A
Absorbance at 405 nm

| Time | Blank | | | 3 cm × 1 cm Control | | |
|---|---|---|---|---|---|---|
| 0 | 0.04430 | 0.04260 | 0.04420 | 0.04430 | 0.04260 | 0.04420 |
| 15 | 0.05450 | 0.04840 | 0.04940 | 0.05290 | 0.05300 | 0.04810 |
| 30 | 0.05520 | 0.05400 | 0.05520 | 0.05530 | 0.05720 | 0.05160 |
| 60 | 0.06710 | 0.06520 | 0.06730 | 0.06180 | 0.06230 | 0.05970 |
| 120 | 0.07800 | 0.07690 | 0.07810 | 0.06770 | 0.06820 | 0.07120 |
| Slope | 0.00027 | 0.00029 | 0.00029 | 0.00018 | 0.00019 | 0.00023 |

TABLE 69B
Absorbance at 405 nm

| | 3 cm × 1 cm Lipase | | | 3 cm × 1 cm Lipase | | |
|---|---|---|---|---|---|---|
| Time | 200 g/gal | | | 100 g/gal | | |
| 0 | 0.04430 | 0.04260 | 0.04420 | 0.04430 | 0.04260 | 0.04420 |
| 15 | 0.07050 | 0.11020 | 0.06940 | 0.05300 | 0.05260 | 0.05300 |
| 30 | 0.07970 | 0.11690 | 0.07850 | 0.06280 | 0.06780 | 0.06270 |
| 60 | 0.10290 | 0.12410 | 0.09510 | 0.09460 | 0.08930 | 0.08780 |
| 120 | 0.13500 | 0.15060 | 0.12870 | 0.10620 | 0.12110 | 0.11940 |
| Slope | 0.00071 | 0.00069 | 0.00065 | 0.00054 | 0.00066 | 0.00064 |

TABLE 70A
Absorbance Averages

Absorbance Average

| Time | Blank | Control | 200 g/gal | 100 g/gal |
|---|---|---|---|---|
| 0 | 0.04370 | 0.04370 | 0.04370 | 0.04370 |
| 15 | 0.05077 | 0.05133 | 0.08337 | 0.05287 |
| 30 | 0.05480 | 0.05470 | 0.09170 | 0.06443 |
| 60 | 0.06653 | 0.06127 | 0.10737 | 0.09057 |
| 120 | 0.07767 | 0.06903 | 0.13810 | 0.11557 |

TABLE 70B
Absorbance Average's Standard
Deviations

Absorbance Deviation

| Time | Blank | Control | 200 g/gal | 100 g/gal |
|---|---|---|---|---|
| 0 | 0.000954 | 0.000954 | 0.000954 | 0.000954 |
| 15 | 0.003272 | 0.002801 | 0.023245 | 0.000231 |
| 30 | 0.000693 | 0.002848 | 0.021832 | 0.002916 |
| 60 | 0.001159 | 0.00138 | 0.015007 | 0.003573 |
| 120 | 0.000666 | 0.001893 | 0.011274 | 0.008156 |

TABLE 71
Absorbance vs. Time Slope

| | Slope | U | | U |
|---|---|---|---|---|
| Sample | (A/min) | (umol/min) | U Average | Deviation |
| Blank | 0.000267 | 0.0584 | 0.06 | 0.00 |
| | 0.000285 | 0.0624 | | |
| | 0.000285 | 0.0625 | | |
| Control 3 cm² | 0.000177 | 0.0388 | 0.04 | 0.01 |
| | 0.000187 | 0.0410 | | |
| | 0.000226 | 0.0494 | | |
| 200 g/gal | 0.000707 | 0.1548 | 0.15 | 0.01 |
| 3 cm² | 0.000687 | 0.1503 | | |
| | 0.000648 | 0.1418 | | |
| 100 g/gal | 0.000540 | 0.1182 | 0.13 | 0.01 |
| 3 cm² | 0.000657 | 0.1437 | | |
| | 0.000639 | 0.1399 | | |

### EXAMPLE 22

This Example demonstrates a lipase assay determining the efficacy of lipase in a coating (e.g., paint). Films of Sherwin-Williams Acrylic Latex comprising lipase were assayed 7 months after they were prepared. Materials used are shown in the table below.

TABLE 72
Materials

200 mM TRIS Buffer (Sigma Product # T1503); brought to pH = 7.1
with HCl
4-nitrophenyl acetate (Sigma Product # N8130) 14.5 mM solution in
isopropyl alcohol
Lipase from porcine pancreas (Sigma Product # L3126)
2 mL microtubes
Pipette
Pipette Tips
Plate Reader
96-well Plate

The reaction procedure included: cutting 1 cm×3 cm free film coupon sizes; placing individual coupons into labeled 2 mL microtubes, with each of the coupon samples tested in triplicate; adding 750 μl 200 mM TRIS to each microtube; adding 600 ul ddH2O to each microtube; adding 150 ul 14.5 mM p-nitrophenyl acetate to each microtube; preparing control samples that had 750 ul 200 mM TRIS, 600 ul ddH2O, and 150 ul 14.5 mM p-nitrophenyl acetate; taking out at each desired time point, 100 ul and reading the absorbance at 405 nm in a 96-well plate; and plotting absorbance vs. time to calculate the slope. Data and calculate values are shown below, demonstrating lipase activity in a cured coating's film 7 months after preparation.

TABLE 73
Absorbance at 405 nm Data

| Time | | | | | | | |
|---|---|---|---|---|---|---|---|
| (min) | Blank | Control | | | Lipase | | |
| 0 | 0.0423 | 0.0423 | 0.0423 | 0.0423 | 0.0423 | 0.0423 | 0.0423 |
| 15 | 0.0477 | 0.0475 | 0.0487 | 0.0495 | 0.1760 | 0.1933 | 0.1719 |
| 30 | 0.0562 | 0.0556 | 0.0550 | 0.0572 | 0.3353 | 0.3631 | 0.3137 |
| 45 | 0.0587 | 0.0598 | 0.0616 | 0.0624 | 0.4642 | 0.5084 | 0.4486 |
| 60 | 0.0643 | 0.0673 | 0.0684 | 0.0691 | 0.6008 | 0.6069 | 0.5565 |
| 90 | 0.0751 | 0.0762 | 0.0785 | 0.0783 | 0.7181 | 0.7896 | 0.7591 |
| Slope | 0.0004 | 0.0004 | 0.0004 | 0.0005 | 0.0095 | 0.0105 | 0.0091 |

TABLE 74
Average pNP Absorbance at 405 nm

| | | Control | Lipase | Control | Lipase |
|---|---|---|---|---|---|
| Time | Blank | Avg | Avg | SD | SD |
| 0 | 0.0423 | 0.0423 | 0.0423 | 0.0000 | 0.0000 |
| 15 | 0.0477 | 0.0486 | 0.1804 | 0.0010 | 0.0114 |
| 30 | 0.0562 | 0.0559 | 0.3374 | 0.0011 | 0.0248 |
| 45 | 0.0587 | 0.0613 | 0.4737 | 0.0013 | 0.0310 |
| 60 | 0.0643 | 0.0683 | 0.5881 | 0.0009 | 0.0275 |
| 90 | 0.0751 | 0.0777 | 0.7556 | 0.0013 | 0.0359 |

TABLE 75
Activity Data

| | Slope | U | | |
|---|---|---|---|---|
| Sample | (A/min) | (umol/min) | U Avg | U SD |
| Blank | 0.0004 | 0.0842 | 0.08 | NA |
| Control | 0.0004 | 0.0884 | 0.09 | 0.01 |
| | 0.0004 | 0.0937 | | |
| | 0.0005 | 0.0992 | | |
| Lipase (100 mg/ml | 0.0095 | 2.0796 | 2.12 | 0.15 |
| wet) | 0.0105 | 2.2884 | | |
| | 0.0091 | 1.9857 | | |

TABLE 76
Absorbance vs. Time Slope

| Sample | U (μmol/min) |
|---|---|
| Blank | 0.08 + 0.00 |
| Control | 0.09 + 0.01 |
| Lipase | 2.12 + 0.15 |

### EXAMPLE 23

This Example demonstrates the use of a coating comprising a lipase, and the enzymatic activity conferred to the coating comprising the lipase by detection of triglyceride breakdown through monitoring pH.

The equipment/reagents were as follows: pH meter; shaker; Lightin Lab Master paint mixer; phenol red (Sigma-Aldrich; Catalog #-P3532), 1.128 mM in distilled water, pH=7.0; lipase (Sigma-Aldrich; Catalog #-L₃₁₂₆), Sherwin Williams acrylic latex paint; sodium hydroxide; hydrochloric acid; isopropyl alcohol; and vegetable oil. The solutions used in measuring pH changes included a phenol red stock solution, 1.128 mM in distilled water, pH=7.0.

The procedure for preparation of the surfaces coated with paint either comprising lipase or not (control paint) was as follows: first, 100 mg/ml, 50 mg/ml, and 0 mg/ml lipase solutions in paint were made; second, solutions were mixed for 3 minutes; third, paints were spread to 8 mils thickness and allowed to dry for 96 hours, and fourth, 1 cm×4 cm coupons were cut from the paint film.

The pre-experimental set-up included the following steps: first, a 1 cm×4 cm piece of film of each lipase concentration was placed in a 15 ml eppendorf tube in triplicate; second, 10 ml ddH₂O was added inside the eppendorf tube; third, tubes on shaker were set for 24 hours, and fourth, after 24 hours, the water from the tube was removed and the film placed in a new 15 ml eppendorf tube. For measuring the control paint (no lipase) samples, the following steps were conducted: first, 5 ml of phenol red stock solution was added into a 15 ml eppendorf tube; second, 5 ml of phenol red stock solution with 100 μl vegetable oil was added into a 15 ml eppendorf tube; third, a 1 cm×4 cm piece of paint film (no lipase) from both the washed and non-washed films was added into a 15 ml eppendorf tube in triplicate; fourth, 5 ml of the phenol red stock solution was added into the 15 ml eppendorf tubes along with 100 μl vegetable oil; and fifth, the tubes were set on a shaker for 24 hours. To measure the paint samples comprising lipase: first, a 1 cm×4 cm piece of the 50 mg/ml paint film, both washed and unwashed, was added into a 15 ml eppendorf tube; second, a 1 cm×4 cm piece of the 100 mg/ml paint film, both washed and unwashed, was added into a 15 ml eppendorf tube; third, 5 ml of the Phenol Red stock solution was added into each tube along with 100 μl vegetable oil; and fourth, the tubes were set on shaker for 24 hours. For both the control paint and lipase paint samples, the pH of each sample was recorded at 24 hours.

Phenol Red is a pH indicator that is yellow in color below pH 6.8 and red in color above pH 8.2. Setting the pH at 7.0 right before the 6.8 end point would demonstrate a color change if the solution becomes slightly more acidic. If in fact the triglycerides are being broken down into free fatty acids by lipase, the pH of the solution should go down, thus exhibiting a color change. In the presence of a paint film with no lipase, the pH of the phenol red solution rose from 7 to almost 9. The pH of the tubes with lipase in them were both substantially lower than the control tubes, demonstrating that the triglycerides were broken down into fatty acids, decreasing the pH of the solutions. All lipase impregnated coatings demonstrated catalytic activity. Washing the coating films with water decreased their effectiveness but the films were still active. Further, vegetable oil was spread over panels that were either control (no lipase) or lipase impregnated. After a day, the lipase impregnated panels were dry while the control panels were still visibly full of oil. It is also contemplated that greater loads of lipase, such as, for example, 200 mg/ml, 100 mg/ml, and 50 mg/ml lipase, may be used.

TABLE 77
Samples

| | No washing cycle | 24 hr washing cycle pH at |
|---|---|---|
| Sample | pH at 24 hr | 24 hr |
| Control | 8.87 + 0.01 | 8.78 + 0.04 |
| 50 mg/ml Lipase | 6.80 + 0.05 | 7.25 + 0.21 |
| 100 mg/ml Lipase | 6.70 + 0.05 | 6.63 + 0.07 |

TABLE 78
pH Buffer

| Sample | pH |
|---|---|
| Phenol Red | 7.07 |
| Phenol Red w/oil | 7.08 |

### EXAMPLE 24

This Example demonstrates the use of a coating comprising a lipase, and the enzymatic activity conferred to the coating comprising the lipase by detection of the hydrolysis of 4-nitrophenyl palmitate through monitoring pH.

The equipment/reagents were as follows: 40 mM CHES Buffer; bring to pH=9.0 with NaOH; 4-nitrophenyl palmitate (Sigma Product # N2752), 14.5 mM solution in isopropyl alcohol; 4-nitrophenyl acetate; lipase from porcine pancreas (Sigma Product # L3126); Sherwin-Williams acrylic latex paint; 2 mL microtubes; paint spreader (1-8 mils); polypropylene blocks; Lightnin Labmaster Mixer; rotator shaker; pipettes and pipetteman; and centrifuge.

The following paint formulations were evaluated: Sherwin-Williams Acrylic Latex Control (no additive), and Sherwin-Williams Acrylic Latex with 100 mg/mL lipase. The paints were mixed in a plastic 50 ml eppendorf tube with a glass stirring rod for three minutes followed by a paint mixer for three minutes. The paints were spread with a mils spreader to 8 mils thickness onto polypropylene surfaces and were allowed to dry a minimum of 72 hours prior to assay. Coupons were generated as free films from the polypropylene surfaces.

The procedure for the preparation of the blank (control) samples was: adding 500 ul 40 mM CHES, 400 ul ddH₂O, and 100 ul 14.5 mM p-nitrophenyl palmitate to a 2 ml microtube. The procedure for preparation of the experimental (comprising lipase) samples was: cutting the following free film sizes for the 100 mg/ml lipase films—1 cm×3 cm, 1 cm×2 cm, and 1 cm×1 cm, and for the control film (no lipase)—1 cm×3 cm; placing the free films into labeled 2 mL microtubes, where each of the coupon sizes were tested in triplicate; adding 500 ul 40 mM CHES to each microtube; adding 400 ul ddH2O to each microtube; adding 100 ul 14.5 mM p-nitrophenyl palmitate to each microtube; and setting microtubes on a shaker. At each time point, tubes were placed in a centrifuge for 5 minutes at 13,000 RPM. A 100 ul was removed from each tube and the absorbance of the reaction product p-nitrophenol read at 405 nm in a 96-well plate.

The tables below shows the activity of each sample. The measured rates of reaction for the free films without any lipase were essentially baseline, exhibiting no destruction of the 4-nitrophenol palmitate. All lipase impregnated coatings demonstrated catalytic activity. The specific activity per centimeter basis was consistent within the different sample sizes.

TABLE 79A
Lipase Activity in Sherwin-Williams Latex pNP Absorbance at 405 nm

| Time | | | | | | | | | |
|---|---|---|---|---|---|---|---|---|---|
| (min) | 1 cm × 3 cm Lipase | | | 1 cm × 2 cm Lipase | | | 1 cm × 1 cm Lipase | | |
| 1 | 0.2314 | 0.3159 | 0.2781 | 0.3146 | 0.4118 | 0.3865 | 0.4265 | 0.3141 | 0.2917 |
| 30 | 0.2511 | 0.3337 | 0.2615 | 0.2850 | 0.3465 | 0.3523 | 0.3849 | 0.2723 | 0.3136 |
| 60 | 0.2625 | 0.3365 | 0.2794 | 0.2984 | 0.3451 | 0.3494 | 0.3833 | 0.2826 | 0.2873 |
| 120 | 0.2674 | 0.3351 | 0.3180 | 0.2960 | 0.3342 | 0.3361 | 0.3680 | 0.2867 | 0.2657 |
| 210 | 0.2949 | 0.3502 | 0.3057 | 0.2946 | 0.3306 | 0.3304 | 0.3527 | 0.2792 | 0.2329 |
| 1200 | 0.4051 | 0.5281 | 0.4568 | 0.3361 | 0.3308 | 0.3374 | 0.3016 | 0.3066 | 0.2159 |

TABLE 79B
Lipase Activity in Sherwin-Williams Latex pNP
Absorbance at 405 nm

| Time (min) | 1 cm × 3 cm Control | | | Blank | | |
|---|---|---|---|---|---|---|
| 1 | 0.3718 | 0.4458 | 0.2327 | 0.3154 | 0.4142 | 0.3773 |
| 30 | 0.3119 | 0.3631 | 0.2172 | 0.2757 | 0.3442 | 0.3069 |
| 60 | 0.2852 | 0.3380 | 0.2025 | 0.2674 | 0.3307 | 0.2767 |
| 120 | 0.2473 | 0.2572 | 0.1707 | 0.2748 | 0.3259 | 0.2780 |
| 210 | 0.1707 | 0.1996 | 0.1542 | 0.2621 | 0.3007 | 0.2616 |
| 1200 | 0.0541 | 0.0552 | 0.0590 | 0.2374 | 0.2640 | 0.2264 |

TABLE 80
Lipase Average Activity in
Sherwin-Williams Latex pNP
Absorbance at 405 nm

| Time | Lipase | Control | |
|---|---|---|---|
| (min) | 1 cm × 3 cm | 1 cm × 3 cm | Blank |
| 1 | 0.2751 | 0.3501 | 0.3690 |
| 30 | 0.2821 | 0.2974 | 0.3089 |
| 60 | 0.2928 | 0.2752 | 0.2916 |
| 120 | 0.3068 | 0.2251 | 0.2929 |
| 210 | 0.3169 | 0.1748 | 0.2748 |
| 1200 | 0.4633 | 0.0561 | 0.2426 |

TABLE 81A
Lipase Activity in Sherwin-Williams Latex pNP Absorbance at 405 nm

| Time | | | | | | | | | |
|---|---|---|---|---|---|---|---|---|---|
| (min) | 1 cm × 3 cm Lipase | | | 1 cm × 2 cm Lipase | | | 1 cm × 1 cm Lipase | | |
| 0 | | | | | | | | | |
| 30 | 0.1685 | 0.2200 | 0.1654 | 0.2135 | 0.1494 | 0.1457 | 0.1271 | 0.0711 | 0.1389 |
| 60 | 0.2287 | 0.1822 | 0.2027 | 0.1570 | 0.2008 | 0.1554 | 0.1500 | 0.1284 | 0.0758 |
| 120 | 0.2044 | 0.2208 | 0.2487 | 0.1694 | 0.1926 | 0.2007 | 0.1126 | 0.0771 | 0.0859 |
| 225 | 0.2521 | 0.2621 | 0.2620 | 0.2707 | 0.1920 | 0.1746 | 0.1779 | 0.1654 | 0.1611 |
| 1200 | 0.3917 | 0.3579 | 0.3735 | 0.2315 | 0.2607 | 0.2682 | 0.1335 | 0.1702 | 0.1300 |

TABLE 81B
Lipase Activity in Sherwin-Williams Latex pNP
Absorbance at 405 nm

| Time | | | | | | |
|---|---|---|---|---|---|---|
| (min) | 1 cm × 3 cm Control | | | Blank | | |
| 0 | | | | 0.1114 | 0.0981 | 0.1269 |
| 30 | 0.1551 | 0.1628 | 0.1173 | 0.1410 | 0.1022 | 0.1204 |
| 60 | 0.1198 | 0.0987 | 0.1029 | 0.0974 | 0.1278 | 0.1119 |
| 120 | 0.1365 | 0.1082 | 0.1192 | 0.1487 | 0.1284 | 0.0995 |
| 225 | 0.0680 | 0.0688 | 0.0602 | 0.1129 | 0.0788 | 0.1231 |
| 1200 | 0.0514 | 0.0521 | 0.0599 | 0.1008 | 0.1106 | 0.0626 |

TABLE 82
Lipase Activity in Sherwin-Williams Latex pNP Average Absorbance at 405 nm and Standard
Deviations

| | Average | | | | | SD | | | | |
|---|---|---|---|---|---|---|---|---|---|---|
| | Lipase | | | Control | | Lipase | | | Control | |
| | 1 cm × | 1 cm × | 1 cm × | 1 cm × | | 1 cm × | 1 cm × | 1 cm × | 1 cm × | |
| Time (min) | 3 cm | 2 cm | 1 cm | 3 cm | Blank | 3 cm | 2 cm | 1 cm | 3 cm | Blank |
| 0 | 0.1121 | 0.1121 | 0.1121 | 0.1121 | 0.1121 | 0.0144 | 0.0144 | 0.0144 | 0.0144 | 0.0144 |
| 30 | 0.1846 | 0.1695 | 0.1124 | 0.1451 | 0.1212 | 0.0307 | 0.0381 | 0.0362 | 0.0244 | 0.0194 |
| 60 | 0.2045 | 0.1711 | 0.1181 | 0.1071 | 0.1124 | 0.0233 | 0.0258 | 0.0382 | 0.0112 | 0.0152 |
| 120 | 0.2246 | 0.1876 | 0.0919 | 0.1213 | 0.1255 | 0.0224 | 0.0162 | 0.0185 | 0.0143 | 0.0247 |
| 225 | 0.2587 | 0.2124 | 0.1681 | 0.0657 | 0.1049 | 0.0057 | 0.0512 | 0.0087 | 0.0048 | 0.0232 |
| 1200 | 0.3744 | 0.2535 | 0.1446 | 0.0545 | 0.0913 | 0.0169 | 0.0194 | 0.0223 | 0.0047 | 0.0254 |

TABLE 83A
Lipase Activity in Sherwin-Williams Latex pNP
Absorbance at 405 nm and Initial Slopes

| | Lipase | | | | | | | | |
|---|---|---|---|---|---|---|---|---|---|
| Time (min) | 1 cm × 3 cm | | | 1 cm × 2 cm | | | 1 cm × 1 cm | | |
| 0 | 0.1121 | 0.1121 | 0.1121 | 0.1121 | 0.1121 | 0.1121 | 0.1121 | 0.1121 | 0.1121 |
| 225 | 0.2521 | 0.2621 | 0.2620 | 0.2707 | 0.1920 | 0.1746 | 0.1779 | 0.1654 | 0.1611 |
| Slope | 0.0006 | 0.0007 | 0.0007 | 0.0007 | 0.0004 | 0.0003 | 0.0003 | 0.0002 | 0.0002 |
| (ΔAbs/Δmin) | | | | | | | | | |
| U (umol/min) | 0.1362 | 0.1459 | 0.1458 | 0.1543 | 0.0777 | 0.0608 | 0.0640 | 0.0519 | 0.0477 |
| U/cm² | 0.0454 | 0.0486 | 0.0486 | 0.0772 | 0.0389 | 0.0304 | 0.0640 | 0.0519 | 0.0477 |

TABLE 83B
Lipase Activity in Sherwin-Williams Latex pNP Absorbance
at 405 nm and Initial Slopes

| Time (min) | 1 cm × 3 cm Control | | | Blank | | |
|---|---|---|---|---|---|---|
| 0 | 0.1121 | 0.1121 | 0.1121 | 0.1121 | 0.1121 | 0.1121 |
| 225 | 0.0680 | 0.0688 | 0.0602 | 0.1129 | 0.0788 | 0.1231 |
| Slope | −0.0002 | −0.0002 | −0.0002 | 0.0000 | −0.0001 | 0.0000 |
| (ΔAbs/Δmin) | | | | | | |
| U (umol/min) | −0.0429 | −0.0421 | −0.0505 | 0.0008 | −0.0324 | 0.0107 |
| U/cm² | | | | | | |

TABLE 84
Sample Activity

| Sample | U (μmol/min) | U (μmol/min)/cm² |
|---|---|---|
| 1 cm × 3 cm; with lipase | 0.1427 ± 0.0056 | 0.0476 ± 0.0019 |
| 1 cm × 2 cm; with lipase | 0.0976 ± 0.0498 | 0.0488 ± 0.0249 |
| 1 cm × 1 cm; with lipase | 0.0545 ± 0.0085 | 0.0545 ± 0.0085 |
| 1 cm × 3 cm; no lipase | −0.0452 ± 0.0046 | |
| Blank | −0.0070 ± 0.0226 | |

The reaction containing the 1 cm×3 cm free-film with lipase went to 50% completion. This is due to the nature of the insolubility of 4-nitrophenyl palmitate. Particles of 4-nitrophenyl palmitate were present in all microtubes due to precipitation when it comes in contacts with water. The 1 cm×1 cm free-film was likely too small a film size, although the microtube was visually yellow, the data did not support the fact that the reaction did in fact take place. 4-nitrophenyl palmitate was originally used, but it self-hydrolyzed in water. Further, vegetable oil was spread over panels that were either control (no lipase) or lipase impregnated. After a day, the lipase impregnated panels were dry while the control panels were still visibly full of oil. It is also contemplated that greater loads of lipase, such as, for example, 200 mg/ml, 100 mg/ml, and 50 mg/ml lipase, may be used.

### EXAMPLE 25

This Example is directed to additional examples of lipolytic enzyme encoding nucleic acid sequences (e.g., full length cDNAs for lipolytic genes) that are contemplated for use in the expression of recombinant lipolytic enzymes, as well as source organisms for endogenously produced lipolytic enzymes, for use in the preparation of biomolecular compositions.

TABLE 85
Lipolytic Enzyme Genes and Source Organisms

| Lipolytic Enzyme | Characteristics | Source | Accession No |
|---|---|---|---|
| carboxylesterase | CXE4 gene | Actinidia deliciosa | DQ279917 |
| carboxylesterase | CXE3 gene | Actinidia deliciosa | DQ279916 |
| carboxylesterase | | Aedes aegypti | XM_001647935 |
| carboxylesterase | carboxylesterase-6 | Aedes aegypti | XM_001656069 |
| carboxylesterase | malathion-resistant | Anisopteromalus | AF064524 |
| | | calandrae | |
| carboxylesterase | malathion-susceptible | Anisopteromalus | AF064523 |
| | | calandrae | |
| carboxylesterase | CarE-S gene | Aphis gossypii | AY049740 |
| carboxylesterase | organophosphorus | Aphis gossypii | AB245435 |
| | insecticide super-susceptible | | |
| | strain | | |
| carboxylesterase | organophosphorus | Aphis gossypii | AB245434 |
| | insecticide susceptible strain | | |
| carboxylesterase | | Arabidopsis thaliana | NM_001036026 |
| carboxylesterase | est-1 gene, GeneID: | Archaeoglobus | NC_000917 |
| | 1484085 | fulgidus | |
| carboxylesterase | estA gene, GeneD: 1484939 | Archaeoglobus | NC_000917 |
| | | fulgidus | |
| carboxylesterase | est-3 gene, GeneID: | Archaeoglobus | NC_000917 |
| | 1485568 | fulgidus | |
| carboxylesterase | est-2 gene, GeneID: | Archaeoglobus | NC_000917 |
| | 1484765 | fulgidus | |
| carboxylesterase | | Aspergillus clavatus | XM_001271426 |
| | | NRRL 1 | |
| carboxylesterase | COE gene | Athalia rosae | AB208651 |
| carboxylesterase | | Bombyx mandarina | EF157830 |
| carboxylesterase | | Bombyx mori | DQ443360 |
| carboxylesterase | carboxylesterase 2, intestine | Bos taurus | BC102288 |
| | liver | | |
| carboxylesterase | | Caenorhabditis | NM_071999 |
| | | elegans B0238.1 | |
| carboxylesterase | | Caenorhabditis | NM_068642 |
| | | elegans C17H12.4 | |
| carboxylesterase | | Caenorhabditis | NM_171976 |
| | | elegans F55F3.2a | |
| carboxylesterase | | Caenorhabditis | NM_068669 |
| | | elegans T22D1.11 | |
| carboxylesterase | CESdD1 gene, | Canis familiaris | AB023629 |
| | carboxylesterase D1 | | |
| carboxylesterase | | Cavia porcellus | AB010634 |
| carboxylesterase | CES1 gene | Felis catus | AB094147 |
| carboxylesterase | CES-K1 gene | Felis catus | AB114676 |
| carboxylesterase | GeneID: 5452002 | Fervidobacterium | NC_009718 |
| | | nodosum Rt17-B1 | |
| carboxylesterase | | Helicoverpa armigera | EF547544 |
| carboxylesterase | carboxylesterase 3, brain | Homo sapiens | BC053670 |
| carboxylesterase | CES5, carboxylesterase 5 | Homo sapiens | AY907669 |
| carboxylesterase | carboxylesterase 2, intestine, | Homo sapiens | BC032095 |
| | liver | | |
| carboxylesterase | | Homo sapiens | D50579 |
| carboxylesterase | carboxylesterase 7 | Homo sapiens | BC117126 |
| carboxylesterase | | Macaca fascicularis | AB010633 |
| carboxylesterase | CXE10 gene | Malus pumila | DQ279911 |
| carboxylesterase | CXE1 gene | Malus pumila | DQ279902 |
| carboxylesterase | | Mesocricetus auratus | D50577 |
| carboxylesterase | | Mus musculus | AB023631 |
| carboxylesterase | carboxylesterase 2 | Mus musculus | BC034182 |
| carboxylesterase | carboxylesterase ML3 | Mus musculus | AB110073 |
| carboxylesterase | | Mus musculus | M57960 |
| carboxylesterase | carboxylesterase 6 | Mus musculus | BC024491 |
| carboxylesterase | carboxylesterase 5 | Mus musculus | BC055062 |
| carboxylesterase | carboxylesterase 3 | Mus musculus | BC019198 |
| carboxylesterase | carboxylesterase 1 | Mus musculus | BC026897 |
| carboxylesterase | MdaE7 gene | Musca domestica | AF133341 |
| carboxylesterase | | Neosartorya fischeri | XM_001260356 |
| | | NRRL 181 | |
| carboxylesterase | Liver | Oryctolagus cuniculus | AF036930 |
| carboxylesterase | CXE gene | Paeonia suffruticosa | EU072921 |
| | | clone 199 | |
| carboxylesterase | Est gene | Pseudomonas | AF228666 |
| | | fluorescens | |
| carboxylesterase | liver microsomal | Rattus norvegicus | U10698 |
| carboxylesterase | kidney microsomal | Rattus norvegicus | U10697 |
| carboxylesterase | CESrRL1 gene | Rattus norvegicus | AB023630 |
| carboxylesterase | ES-4 gene | Rattus norvegicus | BC128711 |
| carboxylesterase | | Rattus norvegicus | AF479659 |
| carboxylesterase | carboxylesterase 3 | Rattus norvegicus | BC061789 |
| carboxylesterase | rCES2 gene | Rattus norvegicus | AB191005 |
| carboxylesterase | | Spodoptera exigua | EF580101 |
| carboxylesterase | | Spodoptera litura | DQ445461 |
| carboxylesterase | SshEstI gene | Sulfolobus shibatae | AB166870 |
| carboxylesterase | GeneID: 1453975 | Sulfolobus | NC_002754 |
| | | solfataricus P2 | |
| carboxylesterase | | Sus scrofa | AF064741 |
| carboxylesterase | GeneID: 2774935 | Thermus thermophilus | NC_005835 |
| | | HB27 | |
| carboxylesterase | GeneID: 2775775 | Thermus thermophilus | NC_005835 |
| | | HB27 | |
| carboxylesterase | GeneID: 3168028 | Thermus thermophilus | NC_006461 |
| | | HB8 | |
| carboxylesterase | CXE1 gene | Vaccinium | DQ279901 |
| | | corymbosum | |
| carboxylesterase | secreted salivary | Xenopsylla cheopis | EF179418 |
| | | clone XC-184 | |
| carboxylesterase/ | GeneID: 3474139 | Sulfolobus | NC_007181 |
| lipase | | acidocaldarius | |
| Lipase | | Aedes aegypti | XM_001651298 |
| Lipase | | Aedes aegypti | XM_001654736 |
| Lipase | Lip gene | Anguilla japonica | AB070722 |
| Lipase | | Antrodia cinnamomea | EF088667 |
| Lipase | | Arabidopsis thaliana | NM_202246 |
| Lipase | lipase 1, LI-tolerant, | Arabidopsis thaliana | NM_111300 |
| | carboxylesterase | | |
| Lipase | extracellular lipase 4; | Arabidopsis thaliana | NM_106241 |
| | acyltransferase/ | | |
| | carboxylesterase/lipase | | |
| Lipase | ATLIP1 gene, lipase 1, | Arabidopsis thaliana | NM_127084 |
| | galactolipase/ | | |
| | phospholipase/lipase | | |
| Lipase | ARAB-1 gene, | Arabidopsis thaliana | NM_102634 |
| | carboxylesterase | | |
| Lipase | | Arabidopsis thaliana | NM_118185 |
| Lipase | DAD1 gene | Arabidopsis thaliana | NM_130045 |
| Lipase | lipase1; carboxylesterase | Arabidopsis thaliana | NM_123464 |
| Lipase | lipB gene | Aspergillus niger | DQ680031 |
| Lipase | lipA gene | Aspergillus niger | DQ680030 |
| Lipase | | Aspergillus tamarii | EU131679 |
| | | isolate FS132 | |
| Lipase | extracellular | Aureobasidium | EU082005 |
| | | pullulans HN2.3 | |
| Lipase | | Avena sativa | AY566266 |
| Lipase | | Bombyx mandarina | AY945212 |
| Lipase | | Bombyx mori | AY945209 |
| Lipase | bile salt-stimulated lipase | Bos Taurus | BT021633 |
| Lipase | lipase 1 gene | Brassica napus | AY866419 |
| Lipase | lipase 2 | Brassica napus | AY870270 |
| lipase | SIL1 gene | Brassica rapa subsp. | AY101366 |
| | | Pekinensis | |
| Lipase | | Caenorhabditis | NM_069722 |
| | | elegans B0035.13 | |
| Lipase | | Chenopodium rubrum | AY299194 |
| Lipase | GeneID: 5292515 | Clostridium | NC_009617 |
| | | beijerinckii NCIMB | |
| | | 8052 | |
| Lipase | GeneID: 5396655 | Clostridium botulinum | NC_009697 |
| | | A str. | |
| Lipase | GeneID: 5395737 | Clostridium botulinum | NC_009697 |
| | | A str. | |
| Lipase | GeneID: 5405010 | Clostridium botulinum | NC_009699 |
| | | F str. Langeland | |
| Lipase | GeneID: 4540684 | Clostridium novyi NT | NC_008593 |
| Lipase | Hepatic | Danio rerio | BC053243 |
| Lipase | Gastric | Danio rerio | BC052131 |
| Lipase | Adipose | Gallus gallus | EU240627 |
| Lipase | FGL4 gene | Gibberella zeae | EU191903 |
| Lipase | FGL2 gene | Gibberella zeae | EU191902 |
| Lipase | | Gossypium hirsutum | EU273289 |
| Lipase | Endothelial | Homo sapiens | AF118767 |
| Lipase | | Homo sapiens | AF225418 |
| Lipase | Endothelial | Homo sapiens | BC060825 |
| Lipase | LIPH gene, lipase H | Homo sapiens | EF186229 |
| Lipase | LIPK gene, lipase K | Homo sapiens | EF426482 |
| Lipase | LIPM gene, lipase M, | Homo sapiens | EF426484 |
| Lipase | Pancreatic | Homo sapiens | BC014309 |
| Lipase | hormone-sensitive | Homo sapiens | BC070041 |
| Lipase | bile salt-stimulated lipase | Homo sapiens | BC042510 |
| Lipase | adipose, ATGL gene | Homo sapiens | AY894804 |
| Lipase | hepatic | Homo sapiens | D83548 |
| Lipase | Lip gene | Kurtzmanomyces sp. | AB073866 |
| | | I-11 | |
| Lipase | | Leishmania infantum | XM_001467534 |
| | | JPCM5 | |
| Lipase | GeneID: 1474518 | Methanosarcina | NC_003552 |
| | | acetivorans | |
| Lipase | pancreatic | Mus musculus | BC061061 |
| Lipase | member H | Mus musculus | BC037489 |
| Lipase | hormone sensitive | Mus musculus | BC021642 |
| Lipase | pancreatic | Mus musculus | AY387690 |
| Lipase | Gastric | Mus musculus | BC061067 |
| Lipase | | Mus musculus | U37386 |
| Lipase | endothelial | Mus musculus | BC020991 |
| Lipase | Liph gene, lipase H | Mus musculus | AY093499 |
| Lipase | hormone-sensitive | Mus musculus | U08188 |
| Lipase | Lipc gene, hepatic | Mus musculus | AY228765 |
| Lipase | endothelial | Mus musculus | AF118768 |
| Lipase | cytotoxic T lymphocyte | Mus musculus | M30687 |
| Lipase | | Mus musculus | AY894805 |
| Lipase | hepatic | Mus musculus | BC094050 |
| Lipase | Lipc gene, hepatic | Mus spretus | AY225159 |
| Lipase | secretory | Neosartorya fischeri | XM_001257303 |
| | | NRRL 181 | |
| Lipase | lacrimal | Oryctolagus cuniculus | AF351188 |
| Lipase | hepatic | Oryctolagus cuniculus | AF041202 |
| Lipase | | Oryctolagus cuniculus | M99365 |
| | | clone TGL-5K | |
| Lipase | alkaline | Penicillium cyclopium | AF274320 |
| Lipase | hepatic | Rattus norvegicus | BC088160 |
| Lipase | Lipg gene, endothelial | Rattus norvegicus | AY916123 |
| Lipase | lipRs gene | Rhizopus stolonifer | DQ139862 |
| Lipase | OBL2 gene | Ricinus communis | AY724687 |
| Lipase | OBL1 gene | Ricinus communis | AY360220 |
| Lipase | | Ricinus communis | EF071862 |
| | | acidic | |
| Lipase | | Samia cynthia ricini | DQ149986 |
| | | strain Banma | |
| lipase | | Schizosaccharomyces | NM_001023305 |
| | | pombe | |
| Lipase | PL-h gene, heart pancreatic | Spermophilus | AF027293 |
| | | tridecemlineatus | |
| Lipase | Pancreatic | Spermophilus | AF395870 |
| | | tridecemlineatus | |
| Lipase | PTL gene, pancreatic | Spermophilus | AF177403 |
| | | tridecemlineatus clone | |
| | | 22A4 | |
| Lipase | PTL gene, pancreatic | Spermophilus | AF177402 |
| | | tridecemlineatus clone | |
| | | 7G5 | |
| Lipase | lipP-1 gene, GeneID: | Sulfolobus | NC_002754 |
| | 1453956 | solfataricus P2 | |
| Lipase | lipP-2 gene, GeneID: | Sulfolobus | NC_002754 |
| | 1453979 | solfataricus P2 | |
| Lipase | ATGL gene, adipose | Sus scrofa | EF583921 |
| Lipase | Lip gene | Thermomyces | AF054513 |
| | | lanuginosus | |
| Lipase | LIP gene | Trichomonas | AY870437 |
| | | vaginalis | |
| Lipase | bile salt-stimulated lipase | Xenopus laevis | BC106664 |
| Lipase | | Xenopus laevis | BC054271 |
| colipase | pancreatic | Homo sapiens | BT006812 |
| colipase | | Homo sapiens | J02883 |
| colipase | pancreatic | Mus musculus | BC042935 |
| colipase | Clps gene | Mus musculus | AF414676 |
| | | C57BL/6J | |
| colipase | Clps gene | Mus musculus | AF414677 |
| | | CAST/Ei | |
| colipase | Pancreatic | Oryctolagus cuniculus | L06329 |
| colipase | Pancreatic | Spermophilus | AF395869 |
| | | tridecemlineatus | |
| colipase | Pancreatic | Sus scrofa | AF148567 |
| lipase/ | GeneID: 5186955 | Clostridium botulinum | NC_009495 |
| acylhydrolase | | A str. | |
| lipoprotein lipase | | Capra hircus | DQ370053 |
| lipoprotein lipase | | Danio rerio | BC064296 |
| lipoprotein lipase | | Felis catus | U42725 |
| lipoprotein lipase | | Homo sapiens | BT006726 |
| lipoprotein lipase | | Mesocricetus auratus | AB194713 |
| lipoprotein lipase | | Mus musculus | BC003305 |
| lipoprotein lipase | | Oncorhynchus mykiss | AF358669 |
| lipoprotein lipase | | Pagrus major | AB054062 |
| lipoprotein lipase | | Papio Anubis | U18091 |
| lipoprotein lipase | | Rattus norvegicus | L03294 |
| lipoprotein lipase | | Sparus aurata | AY495672 |
| lipoprotein lipase | | Sus scrofa breed | AY559454 |
| | | Duroc | |
| lipoprotein lipase | | Sus scrofa breed | AY686761 |
| | | Large White | |
| lipoprotein lipase | | Sus scrofa breed Mei | AY686760 |
| | | Shan | |
| lipoprotein lipase | | Sus scrofa breed | AY559453 |
| | | Tongcheng | |
| lipoprotein lipase | | Thunnus orientalis | AB370192 |
| acylglycerol lipase | | Danio rerio | BC049487 |
| acylglycerol lipase | | Danio rerio clone | AY398382 |
| | | RK135A2B08 | |
| acylglycerol lipase | | Homo sapiens | BC006230 |
| acylglycerol lipase | | Leishmania infantum | XM_001467371 |
| | | JPCM5 | |
| acylglycerol lipase | | Mus musculus | BC057965 |
| acylglycerol lipase | | Rattus norvegicus | BC107920 |
| acylglycerol lipase | Mgl2 gene | Rattus norvegicus | AY081195 |
| hormone sensitive | LIPE gene | Bos taurus | EF140760 |
| lipase | | | |
| hormone sensitive | testicular isoform | Rattus norvegicus | U40001 |
| lipase | | | |
| hormone sensitive | | Rattus norvegicus | BC078888 |
| lipase | | | |
| hormone sensitive | HSL gene | Spermophilus | AF177401 |
| lipase | | tridecemlineatus | |
| hormone sensitive | | Sus scrofa breed | AY686758 |
| lipase | | Large White | |
| hormone sensitive | | Sus scrofa breed Mei | AY686759 |
| lipase | | Shan | |
| hormone sensitive | | Tetrahymena | XM_001031360 |
| lipase | | thermophila SB210 | |
| phospholipase A₁ | | Arabidopsis thaliana | AF421148 |
| phospholipase A₁ | PLA1 gene | Aspergillus oryzae | E16314 |
| phospholipase A₁ | member A | Bos Taurus | BT020950 |
| phospholipase A₁ | phosphatidic acid-preferring | Bos Taurus | AF045022 |
| phospholipase A₁ | | Brassica rapa | EF492990 |
| phospholipase A₁ | intracellular, ipla-1 | Caenorhabditis | EU180219 |
| | | elegans | |
| phospholipase A₁ | PLA1 gene | Capsicum annuum | EF595843 |
| phospholipase A₁ | | Danio rerio | BC066406 |
| phospholipase A₁ | phosphatidylserine-specific, | Homo sapiens | AF035268 |
| phospholipase A₁ | member A | Homo sapiens | BC047703 |
| phospholipase A₁ | | Homo sapiens | E16580 |
| phospholipase A₁ | membrane-bound, | Homo sapiens | AY036912 |
| | phosphatidic acid selective | | |
| phospholipase A₁ | beta, membrane-associated | Homo sapiens | AY197607 |
| phospholipase A₁ | phosphatidylserine-specific, | Homo sapiens | AF035269 |
| | deltaC, PS-PLA1deltaC | | |
| | gene | | |
| phospholipase A₁ | Ps-pla1 gene, | Mus musculus | AF063498 |
| | phosphatidylserine-specific | | |
| phospholipase A₁ | | Mus musculus | BC030670 |
| phospholipase A₁ | | Nicotiana tabacum | AF468223 |
| phospholipase A₁ | | Polistes annularis | AF174527 |
| phospholipase A₁ | venom gland | Polybia paulista | EF101736 |
| phospholipase A₁ | phosphatidylserine-specific | Rattus norvegicus | BC078727 |
| phospholipase A₁ | extracellular | Serratia liquefaciens | M23640 |
| phospholipase A₁ | | Vespula vulgaris | L43561 |
| phospholipase A₂ | | Acanthaster planci | AB211367 |
| phospholipase A₂ | | Adamsia carciniopado | AF347072 |
| phospholipase A₂ | ipla2 gene, 85 kda calcium- | Aedes aegypti | XM_001656230 |
| | independent | | |
| phospholipase A₂ | Isozyme | Aipysurus eydouxii | AY561163 |
| | | clone c10 | |
| phospholipase A₂ | | Apis mellifera | AF438408 |
| phospholipase A₂ | phospholipase A2 alpha | Arabidopsis thaliana | AY344842 |
| phospholipase A₂ | ASPLA1 gene | Austrelaps superbus | AF184127 |
| phospholipase A₂ | | Bitis gabonica | AY429476 |
| phospholipase A₂ | group IVA, PLA2G4A gene | Bos taurus | AY363688 |
| phospholipase A₂ | lysosomal, LPLA2 gene | Bos taurus | AY072914 |
| phospholipase A₂ | Acidic | Bothriechis schlegelii | AY764137 |
| phospholipase A₂ | N6 basic | Bothriechis schlegelii | AY355168 |
| phospholipase A₂ | Hypotensive | Bothrops jararacussu | AY145836 |
| phospholipase A₂ | Myotoxic | Bothrops jararacussu | AY185201 |
| phospholipase A₂ | Cytosolic | BrachyDanio rerio | U10330 |
| phospholipase A₂ | | Bungarus caeruleus | AF297663 |
| phospholipase A₂ | Phospholipase A2 II | Bungarus fasciatus | AF387594 |
| phospholipase A₂ | Antimicrobial | Bungarus fasciatus | DQ868667 |
| phospholipase A₂ | phospholipase A2 I | Bungarus fasciatus | AF387595 |
| phospholipase A₂ | Lysosomal | Canis familiaris | AY217754 |
| phospholipase A₂ | | Cavia sp. | D00740 |
| phospholipase A₂ | | Cerrophidion | AY764139 |
| | | godmani D1E6b | |
| phospholipase A₂ | PLA2 gene | Chlamydomonas | XM_001699805 |
| | | reinhardtii | |
| phospholipase A₂ | ppla2-1 gene | Chrysophrys major | AB050632 |
| phospholipase A₂ | gillpla2 gene | Chrysophrys major | AB050633 |
| phospholipase A₂ | | Chrysophrys major | AB009286 |
| phospholipase A₂ | | Crotalus viridis viridis | AF403137 |
| | | isolate E6h | |
| phospholipase A₂ | | Crotalus viridis viridis | AF403138 |
| | | isolate N6 | |
| phospholipase A₂ | Acidic | Crotalus viridis viridis | AY120875 |
| | | strain E6e | |
| phospholipase A₂ | phospholipase A2-I | Daboia russellii | DQ365974 |
| phospholipase A₂ | Acidic | Daboia russellii from | DQ090659 |
| | | India | |
| phospholipase A₂ | Basic | Daboia russellii from | DQ090660 |
| | | India | |
| phospholipase A₂ | Acidic | Daboia russellii | DQ090654 |
| | | siamensis from | |
| | | Myanmar | |
| phospholipase A₂ | | Daboia russellii | DQ090657 |
| | | siamensis from | |
| | | Myanmar | |
| phospholipase A₂ | group VI, cytosolic, | Danio rerio | BC067375 |
| | calcium-independent | | |
| phospholipase A₂ | group XIIB | Danio rerio | BC093127 |
| phospholipase A₂ | | Echis carinatus | AY268946 |
| phospholipase A₂ | acidic, PLA2-4 gene | Echis carinatus | AF539919 |
| | | sochureki | |
| phospholipase A₂ | acidic, PLA2-5 gene | Echis ocellatus | AF539921 |
| phospholipase A₂ | acidic, PLA2-5 gene | Echis pyramidum | AF539920 |
| | | leakeyi | |
| phospholipase A₂ | plaA gene | Emericella nidulans | AB101663 |
| phospholipase A₂ | Secretory | Equus caballus | EF428565 |
| phospholipase A₂ | PLA2 gene | Equus caballus | AF092539 |
| | | cytosolic | |
| phospholipase A₂ | Cytosolic | Gallus gallus | U10329 |
| phospholipase A₂ | group VI, cytosolic, | Homo sapiens | BC051904 |
| | calcium-independent | | |
| phospholipase A₂ | Ca²⁺-independent, long | Homo sapiens | AF102989 |
| | isoform, iPLA2 gene | | |
| phospholipase A₂ | calcium-independent | Homo sapiens | AF064594 |
| phospholipase A₂ | calcium-independent | Homo sapiens | AB041261 |
| phospholipase A₂ | cPLA2 delta gene; cytosolic | Homo sapiens | AB090876 |
| phospholipase A₂ | beta, cytosolic | Homo sapiens | AF121908 |
| phospholipase A₂ | group XIIB | Homo sapiens | BC093996 |
| phospholipase A₂ | Ca²⁺⁻dependent | Homo sapiens | U03090 |
| phospholipase A₂ | group IVB, cytosolic | Homo sapiens | BC025290 |
| phospholipase A₂ | liver platelet | Homo sapiens | AY656695 |
| phospholipase A₂ | PLA2 gene, group IID | Homo sapiens | AF112982 |
| | secretory | | |
| phospholipase A₂ | | Homo sapiens | AF188625 |
| phospholipase A₂ | group IB, pancreas | Homo sapiens | BC005386 |
| phospholipase A₂ | group IIA, platelets, | Homo sapiens | BC005919 |
| | synovial fluid | | |
| phospholipase A₂ | group IID | Homo sapiens | BC025706 |
| phospholipase A₂ | group XIIA | Homo sapiens | BC017218 |
| phospholipase A₂ | group IVA, cytosolic, | Homo sapiens | BC114340 |
| | calcium-dependent | | |
| phospholipase A₂ | group X | Homo sapiens | BC106731 |
| phospholipase A₂ | group IVC, cytosolic, | Homo sapiens | BC063416 |
| | calcium-independent | | |
| phospholipase A₂ | gamma, cytosolic | Homo sapiens | AF058921 |
| phospholipase A₂ | group IVD, cytosolic | Homo sapiens | BC034571 |
| phospholipase A₂ | group IVE | Homo sapiens | BC101612 |
| phospholipase A₂ | group IVF | Homo sapiens | BC146648 |
| phospholipase A₂ | group V | Homo sapiens | BC036792 |
| phospholipase A₂ | gamma, membrane- | Homo sapiens | AF263613 |
| | associated calcium- | | |
| | independent | | |
| phospholipase A₂ | group III | Homo sapiens | BC025316 |
| phospholipase A₂ | | Lapemis hardwickii | EF405872 |
| phospholipase A₂ | pla2 gene | Laticauda | AB037409 |
| | | semifasciata | |
| phospholipase A₂ | | Micrurus corallines | AY157830 |
| phospholipase A₂ | group IB, pancreas | Mus musculus | BC145908 |
| phospholipase A₂ | Pla2g10 gene; group X | Mus musculus | AF166097 |
| | secreted | | |
| phospholipase A₂ | group V | Mus musculus | BC030899 |
| phospholipase A₂ | group IID | Mus musculus | BC111806 |
| phospholipase A₂ | group IIA, platelets, | Mus musculus | BC045156 |
| | synovial fluid | | |
| phospholipase A₂ | Fksg71 gene, group XIII | Mus musculus | AF339738 |
| | secreted | | |
| phospholipase A₂ | group VI | Mus musculus | BC052845 |
| phospholipase A₂ | group V | Mus musculus | AF162713 |
| phospholipase A₂ | group IVA, cytosolic, | Mus musculus | BC003816 |
| | calcium-dependent | | |
| phospholipase A₂ | group XIIB gene | Mus musculus | BC021592 |
| phospholipase A₂ | Pla2g5 gene, group 5 | Mus musculus | U66873 |
| phospholipase A₂ | Pla2g4e gene, cytosolic | Mus musculus | AB195277 |
| phospholipase A₂ | group XIIA | Mus musculus | BC026812 |
| phospholipase A₂ | Pla2 gene, secretory | Mus musculus | AF112984 |
| phospholipase A₂ | Pla2g4f gene cytosolic | Mus musculus | AB195278 |
| phospholipase A₂ | Lpla2, lysosomal | Mus musculus | AF468958 |
| phospholipase A₂ | sPLA2 gene, mutant | Mus musculus | U32359 |
| | secretory group II | | |
| phospholipase A₂ | non-pancreatic secreted type | Mus musculus | U28244 |
| | II | | |
| phospholipase A₂ | Pla2 gene, pancreatic | Mus musculus | AF187852 |
| phospholipase A₂ | group X | Mus musculus | BC028879 |
| phospholipase A₂ | group IVD | Mus musculus | BC113160 |
| phospholipase A₂ | group IIC | Mus musculus | BC029347 |
| phospholipase A₂ | Pla2 gene, group IID | Mus musculus | AF112983 |
| | secretory | | |
| phospholipase A₂ | group I | Mus musculus | AF162712 |
| phospholipase A₂ | group IVC, cytosolic, | Mus musculus | BC117808 |
| | calcium-independent | | |
| phospholipase A₂ | | Mus musculus | D78647 |
| phospholipase A₂ | Pla2g2f gene, group IIF | Mus musculus | AF166099 |
| | secreted | | |
| phospholipase A₂ | group IVB, cytosolic | Mus musculus | BC016255 |
| phospholipase A₂ | cytosolic, phospholipase A2 | Mus musculus | DQ888308 |
| | beta | | |
| phospholipase A₂ | Pla2g2e gene, group IIE | Mus musculus | AF166098 |
| | secreted | | |
| phospholipase A₂ | Pla2g4d gene, cytosolic | Mus musculus | AB195276 |
| phospholipase A₂ | 85 kDa calcium-independent | Mus musculus | U88624 |
| phospholipase A₂ | testis-specific low molecular | Mus musculus | U18119 |
| | weight | | |
| phospholipase A₂ | group IIF | Mus musculus | BC125567 |
| phospholipase A₂ | group IIE | Mus musculus | BC027524 |
| phospholipase A₂ | group III | Mus musculus | BC079556 |
| phospholipase A₂ | group XII-1 | Mus musculus strain | AY007381 |
| | | AKR | |
| phospholipase A₂ | | Mytilus edulis | DQ172904 |
| phospholipase A₂ | NnkPLA-II gene | Naja kaouthia | AB011389 |
| phospholipase A₂ | pla2 gene, clone 1 | Naja naja | L42006 |
| phospholipase A₂ | t1pla2 gene | Nicotiana tabacum | AB190177 |
| phospholipase A₂ | APLA2-1 gene, acidic | Ophiophagus Hannah | AF302908 |
| phospholipase A₂ | PLA2 gene | Ophiophagus Hannah | AF297034 |
| phospholipase A₂ | | Ornithodoros parkeri | EF633936 |
| | | clone OP-525 | |
| phospholipase A₂ | PLA2 gene, microsomal- | Oryctolagus cuniculus | AY739721 |
| | bound CA²⁺-independent | | |
| phospholipase A₂ | group VIB calcium- | Oryctolagus cuniculus | AY738591 |
| | independent | | |
| phospholipase A₂ | group VIA2 | Oryctolagus cuniculus | AY744674 |
| phospholipase A₂ | inpla2 gene | Pagrus major | AB236358 |
| phospholipase A₂ | | Patiria pectinifera | AB022278 |
| phospholipase A₂ | PLA2 gene | Polyandrocarpa | AB107990 |
| | | misakiensis | |
| phospholipase A₂ | | Protobothrops | DQ299948 |
| | | mucrosquamatus | |
| phospholipase A₂ | group V | Rattus norvegicus | BC085745 |
| phospholipase A₂ | group 2C | Rattus norvegicus | BC097325 |
| phospholipase A₂ | aiPLA2 gene, acidic | Rattus norvegicus | AF014009 |
| | calcium-independent | | |
| phospholipase A₂ | group IID | Rattus norvegicus | BC091221 |
| phospholipase A₂ | Pancreatic | Rattus norvegicus | D00036 |
| phospholipase A₂ | group IVA, cytosolic, | Rattus norvegicus | BC070940 |
| | calcium-dependent | | |
| phospholipase A₂ | group IVA, cytosolic, | Rattus norvegicus | BC070940 |
| | calcium-dependent | | |
| phospholipase A₂ | 14 kDa | Rattus norvegicus | U07798 |
| phospholipase A₂ | calcium-independent | Rattus norvegicus | U97146 |
| phospholipase A₂ | group VI | Rattus norvegicus | BC081916 |
| phospholipase A₂ | group X secreted | Rattus norvegicus | AF166100 |
| phospholipase A₂ | Lysosomal | Rattus norvegicus | AY490816 |
| phospholipase A₂ | Cytosolic | Rattus norvegicus | U38376 |
| phospholipase A₂ | | Sistrurus catenatus | AY508692 |
| | | tergeminus | |
| phospholipase A₂ | N6a gene, basic | Sistrurus catenatus | AY355170 |
| | | tergeminus | |
| phospholipase A₂ | G6D49 gene | Trimeresurus | AY355179 |
| | | borneensis | |
| phospholipase A₂ | Acidic | Trimeresurus | AY355178 |
| | | borneensis E6 | |
| phospholipase A₂ | | Trimeresurus | D10070 |
| | | flavoviridis | |
| phospholipase A₂ | Acidic | Trimeresurus gracilis | AY764141 |
| phospholipase A₂ | cTgPLA2-I gene | Trimeresurus | D31774 |
| | | gramineus | |
| phospholipase A₂ | | Trimeresurus | D49388 |
| | | okinavensis | |
| phospholipase A₂ | Acidic | Trimeresurus | AY355174 |
| | | puniceus E6a | |
| phospholipase A₂ | | Trimeresurus | AY355173 |
| | | puniceus G6D49 | |
| phospholipase A₂ | | Trimeresurus | AY211934 |
| | | stejnegeri | |
| phospholipase A₂ | group XIII | Tuber borchii | AF162269 |
| phospholipase A₂ | | Urticina crassicornis | EU003992 |
| phospholipase A₂ | II | Vipera russelli | AY286006 |
| | | siamensis | |
| phospholipase A₂ | I | Vipera russelli | AY256974 |
| | | siamensis | |
| phospholipase A₂ | group IVA, cytosolic, | Xenopus laevis | BC056041 |
| | calcium-dependent | | |
| phospholipase A₂ | group 6, cytosolic, calcium- | Xenopus tropicalis | BC123949 |
| | independent | | |
| phospholipase A₂ | group IVB, cytosolic | Xenopus tropicalis | BC087993 |
| phospholipase C | phospholipase C gamma | Aedes aegypti | XM_001649088 |
| phospholipase C | | Aedes aegypti | XM_001660587 |
| phospholipase C | phospholipase C beta | Aedes aegypti | XM_001653756 |
| phospholipase C | | Aplysia californica | DQ397516 |
| phospholipase C | phosphatidylglycerol | Arabidopsis thaliana | AB084296 |
| | specific, clone: PC-PLC6 | | |
| | gene | | |
| phospholipase C | phospholipase C4, | Arabidopsis thaliana | NM_111224 |
| | nonspecific, NPC4 gene | | |
| phospholipase C | | Arabidopsis thaliana | NM_101237 |
| phospholipase C | ATPLC1 gene | Arabidopsis thaliana | NM_125254 |
| phospholipase C | phospholipase C-gamma | Asterina miniata | AY486068 |
| phospholipase C | Zeta | Bos taurus | BC114836 |
| phospholipase C | delta 1 | Bos taurus | BC133304 |
| phospholipase C | Beta | Caenorhabditis | AF188477 |
| | | elegans | |
| phospholipase C | Gamma | Chaetopterus | EF185302 |
| | | pergamentaceus | |
| phospholipase C | | Chlamydomonas | XM_001696450 |
| | | reinhardtii | |
| phospholipase C | zeta, plcz gene | Coturnix japonica | AB369537 |
| phospholipase C | plc-21 gene | D. melanogaster | M60452 |
| phospholipase C | norpA gene | D. melanogaster | J03138 |
| phospholipase C | plc-21 gene | D. melanogaster | M60453 |
| phospholipase C | beta 3, plcb3 gene | Danio rerio | EF204528 |
| phospholipase C | gamma 1, plcg1 gene | Danio rerio | AY163168 |
| phospholipase C | phosphoinositide-specific, | Dictyostelium | M95783 |
| | DdPLC gene | discoideum | |
| phospholipase C | phosphoinositide-specific, | Dictyostelium | XM_629474 |
| | pipA gene | discoideum AX4 | |
| phospholipase C | gamma D | Drosophila | D29806 |
| | | melanogaster | |
| phospholipase C | zeta, PLCZ1 gene | Gallus gallus | AY843531 |
| phospholipase C | beta isoform, PLC gene | Homarus americanus | AF128539 |
| phospholipase C | beta 2 | Homo sapiens | BT006905 |
| phospholipase C | pancreas-enriched | Homo sapiens | AF117948 |
| phospholipase C | phosphoinositide-specific, | Homo sapiens | AF190642 |
| | PLC-epsilon | | |
| phospholipase C | beta 4, PLCB4 gene | Homo sapiens | L41349 |
| phospholipase C | delta 1 | Homo sapiens | BC050382 |
| phospholipase C | | Homo sapiens | D42108 |
| phospholipase C | epsilon 1 | Homo sapiens | BC151854 |
| phospholipase C | zeta 1 | Homo sapiens | BC125067 |
| phospholipase C | | Loligo pealei | AF258528 |
| phospholipase C | phospholipase C beta | Lytechinus pictus | AY550251 |
| phospholipase C | phospholipase C beta, | Meleagris gallopavo | U49431 |
| | erythrocyte | | |
| phospholipase C | phospholipase C-delta1 | Misgurnus mizolepis | AY134493 |
| phospholipase C | beta 1 | Mus musculus | BC058710 |
| phospholipase C | delta 4 | Mus musculus | AY033991 |
| phospholipase C | beta3 | Mus musculus | U43144 |
| phospholipase C | eta1c gene | Mus musculus | AY691174 |
| phospholipase C | eta1b gene | Mus musculus | AY691173 |
| phospholipase C | eta1a gene | Mus musculus | AY691172 |
| phospholipase C | beta-1b gene | Mus musculus | U85713 |
| phospholipase C | eta 1 gene | Mus musculus | BC042549 |
| phospholipase C | delta 1 gene | Mus musculus | BC015249 |
| phospholipase C | delta 3 gene | Mus musculus | BC031392 |
| phospholipase C | phosphatidylinositol- | Mus musculus | BC039627 |
| | specific, X domain | | |
| | containing 1 | | |
| phospholipase C | beta 3 | Mus musculus | BC035928 |
| phospholipase C | PLC-L2 gene | Mus musculus | AB033615 |
| phospholipase C | delta 4 | Mus musculus | BC066156 |
| phospholipase C | Gamma | Mus musculus | BC023877 |
| phospholipase C | gamma 1 | Mus musculus | BC065091 |
| phospholipase C | Zeta | Mus musculus | BC106768 |
| phospholipase C | alpha | Mus musculus | M73329 |
| phospholipase C | beta 4 | Mus musculus | BC129883 |
| phospholipase C | beta-1a | Mus musculus | U85712 |
| phospholipase C | eta2, Plc-eta2 gene | Mus musculus strain | DQ176851 |
| | | C57BL/6J | |
| phospholipase C | beta 4, Plcb4 gene | Mus musculus strain | AF332072 |
| | | ILS | |
| phospholipase C | beta 1 | Mus musculus strain | AF498250 |
| | | ISS | |
| phospholipase C | phospholipase C2 | Nicotiana tabacum | AF223573 |
| phospholipase C | PLC3 gene | Nicotiana tabacum | EF043044 |
| phospholipase C | phosphoinositide-specific | Nicotiana tabacum | EF520286 |
| phospholipase C | phosphoinositide-specific | Oryza sativa | AF332874 |
| phospholipase C | beta 2, plcb2 gene | Oryzias latipes | AB254242 |
| phospholipase C | | Petunia inflate | DQ322461 |
| phospholipase C | ISC1 gene | Pichia stipitis CBS | XM_001385548 |
| | | 6054 | |
| phospholipase C | sphingomyelin/lysocholinephospholipid | Plasmodium | AF323591 |
| | | falciparum | |
| phospholipase C | Zeta | Rattus norvegicus | AY885259 |
| phospholipase C | delta4 | Rattus norvegicus | D50455 |
| phospholipase C | splice variant PLC-b4b | Rattus norvegicus | U57836 |
| | gene, brain | | |
| phospholipase C | delta 1, long form | Rattus norvegicus | EF089258 |
| phospholipase C | delta-4 | Rattus norvegicus | U16655 |
| phospholipase C | beta4 | Rattus norvegicus | L15556 |
| phospholipase C | delta isoform, PLCdsu gene | Strongylocentrotus | AY465426 |
| | | purpuratus | |
| phospholipase C | delta 4 | Sus scrofa | AF498759 |
| phospholipase C | PLC1 gene | Torenia fournieri | EU082202 |
| phospholipase C | PLC gene | Torenia fournieri | EF198328 |
| phospholipase C | delta 1 | Toxoplasma gondii | AY830139 |
| phospholipase C | | Watasenia scintillans | AB040460 |
| phospholipase C | gamma-1a | Xenopus laevis | BC070837 |
| phospholipase C | gamma-1b | Xenopus laevis | BC068831 |
| phospholipase C | gamma-1, XPLCG1a gene | Xenopus laevis | AB287408 |
| phospholipase C | PLC gene | Zea mays | AY536525 |
| phospholipase D | | Aedes aegypti | XM_001654711 |
| phospholipase D | AtPLDdelta gene | Arabidopsis thaliana | AB031047 |
| phospholipase D | PLDbeta gene | Arabidopsis thaliana | U84568 |
| phospholipase D | phospholipase D alpha 1, | Arachis hypogaea | AB232321 |
| | plda1 gene | | |
| phospholipase D | PLD gene | Arachis hypogaea | AY274834 |
| phospholipase D | phospholipase D1, | Bos taurus | BC150123 |
| | phosphatidylcholine-specific | | |
| phospholipase D | phosphatidylinositolglycan- | Bos taurus | M60804 |
| | specific | | |
| phospholipase D | N-acyl- | Bos taurus | BT021908 |
| | phosphatidylethanolamine- | | |
| | hydrolyzing, NAPE-PLD | | |
| | gene | | |
| phospholipase D | phospholipase D2, PLD2 | Bos taurus | BT026202 |
| | gene | | |
| phospholipase D | phospholipase D1, PLD1 | Brassica oleracea | AF090445 |
| | gene | | |
| phospholipase D | phospholipase D2, PLD2 | Brassica oleracea | AF090444 |
| | gene | | |
| phospholipase D | PLD gene | Caenorhabditis | AB028889 |
| | | elegans | |
| phospholipase D | PLD1 gene, phospholipase | Cricetulus griseus | U94995 |
| | D1 | | |
| phospholipase D | PLDa1 gene, phospholipase | Cucumis melo var. | DQ267933 |
| | D-alpha | inodorus | |
| phospholipase D | | Cucumis sativus | EF363796 |
| phospholipase D | glycosylphosphatidylinositol, | Dictyostelium | XM_637715 |
| | pldG gene | discoideum AX4 | |
| phospholipase D | phospholipase D3 gene | Dictyostelium | XM_632022 |
| | | discoideum AX4 | |
| phospholipase D | phospholipase D1 gene | Dictyostelium | XM_635684 |
| | | discoideum AX4 | |
| phospholipase D | | Drosophila | AF228314 |
| | | melanogaster | |
| phospholipase D | pldA gene | Emericella nidulans | AB092651 |
| phospholipase D | Alpha | Fragaria × ananassa | AY758359 |
| phospholipase D | beta 1 isoform 1a | Gossypium hirsutum | AY138249 |
| phospholipase D | Alpha | Gossypium hirsutum | EF378946 |
| phospholipase D | | Gossypium hirsutum | AF159139 |
| phospholipase D | delta isoform | Gossypium hirsutum | AF544228 |
| phospholipase D | | Homo sapiens | AF035483 |
| phospholipase D | N-acyl- | Homo sapiens | BC071604 |
| | phosphatidylethanolamine- | | |
| | hydrolyzing, cDNA clone | | |
| | MGC: 87594 | | |
| | IMAGE: 4375696 | | |
| phospholipase D | phosphatidylcholine-specific | Homo sapiens | BC068976 |
| phospholipase D | N-acyl- | Homo sapiens | AB112352 |
| | phosphatidylethanolamine- | | |
| | hydrolyzing | | |
| phospholipase D | PLD gene | Lolium temulentum | EU293806 |
| phospholipase D | TPLD gene | Lycopersicon | AF154425 |
| | | esculentum | |
| phospholipase D | | Mus musculus | BC068144 |
| phospholipase D | N-acyl- | Mus musculus | AB112350 |
| | phosphatidylethanolamine- | | |
| | hydrolyzing | | |
| phospholipase D | Glycosylphosphatidylinositol | Mus musculus | AY081194 |
| phospholipase D | mPLD1 gene, | Mus musculus | U87868 |
| | phosphatidylcholine-specific | | |
| | phospholipase | | |
| phospholipase D | mPLD2 gene, | Mus musculus | U87557 |
| | phosphatidylcholine-specific | | |
| | phospholipase D2 | | |
| phospholipase D | japonica cultivar-group | Oryza sativa | D73411 |
| phospholipase D | PLD1 gene | Papaver somniferum | AF451979 |
| phospholipase D | PLD2 gene | Papaver somniferum | AF451980 |
| phospholipase D | PLD gene | Paralichthys | AY396567 |
| | | olivaceus | |
| phospholipase D | SPO14 gene | Pichia stipitis CBS | XM_001387066 |
| | | 6054 | |
| phospholipase D | PBPLD gene | Pimpinella | U96438 |
| | | brachycarpa | |
| phospholipase D | rPLD1 gene | Rattus norvegicus | U69550 |
| phospholipase D | PLDs gene | Rattus norvegicus | AF017251 |
| phospholipase D | 1a | Rattus norvegicus | AB003170 |
| phospholipase D | 2 | Rattus norvegicus | AB003172 |
| phospholipase D | N-acyl- | Rattus norvegicus | AB112351 |
| | phosphatidylethanolamine- | | |
| | hydrolyzing | | |
| phospholipase D | 1b | Rattus norvegicus | AB003171 |
| phospholipase D | | Rattus norvegicus | AB000779 |
| phospholipase D | | Ricinus communis | L33686 |
| phospholipase D | | Vigna unguiculata | U92656 |
| phospholipase D | alpha, PLD gene | Vitis vinifera | DQ333882 |
| phospholipase D | | Zea mays | D73410 |
| phosphoinositide | | Arabidopsis thaliana | NM_001037020 |
| phospholipase C | | | |
| phosphoinositide | | Aspergillus clavatus | XM_001272056 |
| phospholipase C | | NRRL 1 | |
| phosphoinositide | | Aspergillus fumigatus | XM_746538 |
| phospholipase C | | Af293 | |
| phosphoinositide | PLC gene | Brassica napus | AF108123 |
| phospholipase C | | | |
| phosphoinositide | gamma 2 | Homo sapiens | BC007565 |
| phospholipase C | | | |
| phosphoinositide | beta 1 | Homo sapiens | BC069420 |
| phospholipase C | | | |
| phosphoinositide | | Leishmania infantum | XM_001465631 |
| phospholipase C | | JPCM5 | |
| phosphoinositide | PLC-epsilon | Mus musculus | AB076247 |
| phospholipase C | | | |
| phosphoinositide | | Neosartorya fischeri | XM_001266832 |
| phospholipase C | | NRRL 181 | |
| phosphoinositide | PpPLC2 gene | Physcomitrella patens | AB117760 |
| phospholipase C | | | |
| phosphoinositide | PLC-1 gene | Pichia stipitis CBS | XM_001383864 |
| phospholipase C | | 6054 | |
| phosphoinositide | Epsilon | Rattus norvegicus | AF323615 |
| phospholipase C | | | |
| phosphoinositide | | Toxoplasma gondii | AY304575 |
| phospholipase C | | | |
| phosphoinositide | | Trypanosoma brucei | AY157307 |
| phospholipase C | | | |
| phosphoinositide | | Vigna unguiculata | U85250 |
| phospholipase C | | | |
| phosphoinositide | beta 1 | Xenopus tropicalis | BC118793 |
| phospholipase C | | | |
| phosphoinositide | | Zea mays | EF136661 |
| phospholipase C | | | |
| phospholipase/ | GeneID: 5826212 | Chloroflexus | NC_010175 |
| carboxylesterase | | aurantiacus J-10-fl | |
| phospholipase/ | GeneID: 5452119 | Fervidobacterium | NC_009718 |
| carboxylesterase | | nodosum Rt17-B1 | |
| phospholipase/ | GeneID: 4116934 | Rubrobacter | NC_008148 |
| carboxylesterase | | xylanophilus | |
| phospholipase | Pha2 gene, heterodimeric | Anuroctonus | EF364040 |
| | | phaiodactylus | |
| phospholipase | | Caenorhabditis | NM_061318 |
| | | elegans C03H5.4 | |
| phospholipase | | Caenorhabditis | NM_059984 |
| | | elegans F36A2.9a | |
| phospholipase | | Caenorhabditis | NM_068812 |
| | | elegans R05G6.8 | |
| phospholipase | | Caenorhabditis | NM_064039 |
| | | elegans W02B12.1 | |
| phospholipase | serine dependent | Homo sapiens | U89386 |
| phospholipase | PLDb1 gene | Lycopersicon | AY013255 |
| | | esculentum | |
| phospholipase | PLDa1 gene | Lycopersicon | AY013252 |
| | | esculentum | |
| phospholipase | | Rattus norvegicus | U03763 |
| phospholipase | C-zeta, plcz gene | Sus scrofa | AB113581 |
| lysophospholipase | plb1 gene | Aedes aegypti | XM_001651691 |
| lysophospholipase | | Argas monolakensis | DQ886863 |
| lysophospholipase | | Aspergillus clavatus | XM_001271762 |
| | | NRRL 1 | |
| lysophospholipase | | Aspergillus fumigatus | XM_746859 |
| | | Af293 | |
| lysophospholipase | plb1 gene | Aspergillus fumigatus | AY376592 |
| | | CBS14489 | |
| lysophospholipase | lysophospholipase 3, | Bos Taurus | BT021838 |
| | lysosomal phospholipase A2 | | |
| lysophospholipase | lysophospholipase I | Bos Taurus | BC105143 |
| lysophospholipase | PLB gene | Cavia porcellus | AF045454 |
| lysophospholipase | | Danio rerio | BC092832 |
| lysophospholipase | Plb gene | Dictyostelium | AF411829 |
| | | discoideum | |
| lysophospholipase | plbA gene | Dictyostelium | XM_637741 |
| | | discoideum AX4 | |
| lysophospholipase | | Emericella nidulans | AB193027 |
| lysophospholipase | | Emericella nidulans | AB193027 |
| lysophospholipase | | Giardia lamblia | XM_001709168 |
| | | ATCC 50803 | |
| lysophospholipase | | Homo sapiens | BC042674 |
| lysophospholipase | lysophospholipase II | Homo sapiens | BC017193 |
| lysophospholipase | LPL-I gene, | Homo sapiens | AF090423 |
| | lysophospholipase | | |
| lysophospholipase | LPL1 gene | Homo sapiens | AF081281 |
| lysophospholipase | lysophospholipase 3, | Homo sapiens | BC062605 |
| | lysosomal phospholipase A2 | | |
| lysophospholipase | PLB gene | Monodelphis | DQ875604 |
| | | domestica | |
| lysophospholipase | lysophospholipase II | Mus musculus | AB009653 |
| lysophospholipase | lysophospholipase I | Mus musculus | U89352 |
| lysophospholipase | lysophospholipase 2 | Mus musculus | BC068120 |
| lysophospholipase | lysophospholipase 1 | Mus musculus | BC013536 |
| lysophospholipase | lysophospholipase 3 | Mus musculus | BC019373 |
| lysophospholipase | | Mus musculus | BC033606 |
| lysophospholipase | | Neosartorya fischeri | XM_001266396 |
| | | NRRL 181 | |
| lysophospholipase | Plb gene | Pichia jadinii | AB114901 |
| lysophospholipase | lysophospholipase, PLB4 | Pichia stipitis CBS | XM_001382254 |
| | gene | 6054 | |
| lysophospholipase | PLB1 gene | Pichia stipitis CBS | XM_001383823 |
| | | 6054 | |
| lysophospholipase | PLB6 gene | Pichia stipitis CBS | XM_001385976 |
| | | 6054 | |
| lysophospholipase | 2 | Rattus norvegicus | BC070503 |
| lysophospholipase | | Rattus norvegicus | AB009372 |
| lysophospholipase | lysophospholipase II | Rattus norvegicus | AB021645 |
| lysophospholipase | 3 | Rattus norvegicus | BC098894 |
| lysophospholipase | 1 | Rattus norvegicus | BC085750 |
| lysophospholipase | | Rattus norvegicus | BC098655 |
| lysophospholipase | Liver | Rattus norvegicus | D63885 |
| lysophospholipase | | Rattus norvegicus | D63648 |
| lysophospholipase | | Schistosoma | AF091539 |
| | | japonicum | |
| lysophospholipase | nte1 gene | Schizosaccharomyces | NM_001023078 |
| | | pombe | |
| lysophospholipase | | Sclerotinia | XM_001594173 |
| | | sclerotiorum 1980 | |
| lysophospholipase | II | Xenopus tropicalis | BC075270 |
| sterol esterase | | Rattus norvegicus | BC072532 |
| retinyl palmitate | type 1 | Bos Taurus | BC102781 |
| esterase | | | |
| lipolytic enzyme | GeneID: 5825102 | Chloroflexus | NC_010175 |
| | | aurantiacus J-10-fl | |
| lipolytic enzyme | GeneID: 5824919 | Chloroflexus | NC_010175 |
| | | aurantiacus J-10-fl | |
| lipolytic enzyme | GeneID: 5291607 | Clostridium | NC_009617 |
| | | beijerinckii | |
| lipolytic enzyme | GeneID: 5744860 | Clostridium | NC_010001 |
| | | phytofermentans | |
| lipolytic enzyme | GeneID: 5743766 | Clostridium | NC_010001 |
| | | phytofermentans | |
| lipolytic enzyme | GeneID: 5452570 | Fervidobacterium | NC_009718 |
| | | nodosum Rt17-B1 | |
| lipolytic enzyme | GeneID: 4462758 | Methanosaeta | NC_008553 |
| | | thermophila PT | |
| lipolytic enzyme | GeneID: 1474583 | Methanosarcina | NC_003552 |
| | | acetivorans | |
| lipolytic enzyme | GeneID: 1475504 | Methanosarcina | NC_003552 |
| | | acetivorans | |

### EXAMPLE 26

This Example is directed to the assay for active phosphoric triester hydrolase expression in cells. Routine analysis of parathion hydrolysis in whole cells is accomplished by suspending cultures in 10 milli-Molar (“mM”) Tris hydrocholoride at pH 8.0 comprising 1.0 mM sodium EDTA (“TE buffer”). Cell-free extracts are assayed using sonicated extracts in 0.5 milliLiters (“ml”) of TE buffer. The suspended cells or cell extracts are incubated with 10 microLiters (“μl”) of substrate, specifically 100 μg of parathion in 10% methanol, and p-nitrophenol production is monitored at a wavelength of 400 nm. To induce the opd gene under lac control, 1.0 μmol of isopropyl-β-D-thiogalactopyranoside (Sigma) per ml is added to the culture media.

### EXAMPLE 27

This Example is directed to the preparation of an enzyme powder. In a typical preparation, a single colony of bacteria that expresses the opd gene is selected and cultured in a rich media. After growth to saturation, the cells are concentrated by centrifugation at 7000 rotations per minute (“rpm”) for 10 minutes for example. The cell pellet is then resuspended in a volatile organic solvent such as acetone one or two times to desiccate the cells and to remove a substantial portion of the water contained in the cell pellet. The pellet may then be ground or milled to a powder form. The powder may be frozen or stored at ambient conditions for future use, or may be added immediately to a surface coating formulation. Additionally, the powder may be freeze dried, combined with a cryoprotectant (e.g., cryopreservative), or a combination thereof.

### EXAMPLE 28

This Example is directed to the formation of an OPH powder and latex coating. In an example of use of the powder prepared as described in Example 27, 3 mg of the milled powder was added to 3 ml of 50% glycerol. The suspension was then added to 100 ml of Olympic® premium interior flat latex paint (Olympic®, One PPG Place, Pittsburg, Pa. 15272 USA). This paint with biomolecular composition was then used to demonstrate the activity of the paint biomolecular composition in hydrolysis of a pesticide or a nerve agent analog.

### EXAMPLE 29

This Example demonstrates, in a first set of assays, a paint product as prepared in Example 28 was applied to a hard, metal surface. The surface used in the present Example was a non-galvanized steel surface that was cleaned through being degreased, and pretreated with a primer coat. A control surface was painted with the identical paint with no biomolecular composition. Paraoxon, an organophosphorus nerve gas analog was used as an indicator of enzyme activity. Paraoxon, which is colorless, is degraded to form p-nitrophenol, which is yellow in color, plus diethyl phosphate, thus giving a visual indication of enzyme activity. In multiple assays, the surface with control paint remained white, indicating no production of p-nitrophenol, and the surface painted with the paint and biomolecular composition turned yellow within minutes, indicating an active OPH enzyme in the paint. This demonstration has shown that the surface remains active for more than 65 days, which was the maximum duration of the protocol.

In a further demonstration, the surfaces were treated as described above and each surface was then treated with paraoxon, an OP insecticide. Approximately 100 flies were then placed on each surface under a plastic cover. In each procedure, within three hours, virtually all the flies on the control surface with no paint biomolecular composition were killed by the paraoxon. In contrast, approximately 5% of the flies on the enzyme comprising surface had died.

In a demonstration of enzyme stability in the paint, a series of wood dowels were dipped into the paint comprising OPH enzyme composition. The dowels were then placed in tubes containing paraoxon to indicate enzyme activity as described above. In each case, a positive yellow color was seen except in those dowels painted with no biomolecular composition as controls. The control solution remained clear in every case.

To demonstrate the shelf life of both the dry biomolecular composition and the paint with biomolecular composition, the biomolecular composition was aged from 0 to 20 days prior to mixing in the paint. The mixed paint and biomolecular composition was then also aged from 0 to 20 prior to painting individual dowels. The enzyme composition retained strong activity after 20 days aging prior to being mixed in the paint, and for 20 days after mixing the maximum time used in the assay.

### EXAMPLE 30

This Example relates to a buffered enzyme. As the hydrolysis reaction that degrades nerve agents proceeds, the local pH decreases. Without being limited to any particular mechanism, it is contemplated that due to the law of mass action, or to the optimum pH of the enzyme, the reaction is slower as the pH decreases. Because this effect could prevent or inhibit some surfaces from becoming completely decontaminated, active paint formulations have been prepared that include one or more buffering agents.

In initial procedures, the following compositions were used: 10 mg enzyme powder as described in Example 27, 100 μl 0.1 M buffer, 800 μl H₂O, and 100 μl paraoxon for a 1000 μl reaction volume.

Reactions were run for 1.5 to 2 hours and both pH and product concentration were measured. The concentration of product (p-nitrophenol) is measured by absorbance at 400 nm.

Ammonium bicarbonate, both monobasic and dibasic phosphate buffers, Trizma base and five zwitterionic buffers have been used in the active paint compositions. All the buffers were effective at allowing the reaction to proceed further to completion, thus demonstrating the function of addition of a buffering agent to the active paint compositions.

### EXAMPLE 31

This Example relates to a NATO demonstration of Soman detoxification using an OPH coated surface. At the Sep. 22, 2002, meeting of the NATO Army Armaments Group in Cazaux, France, painted metal surfaces were assayed with soman using standard NATO procedures and protocols. For the assays, 10 cm×10 cm metal plates primed with standard NATO specification paints were coated with paint containing OPH. Control plates plus two different versions of the OPH enzyme composition differing in soman detoxification specificity were used. These surfaces were allowed to dry for several hours at room temperature and then assayed according to standard NATO assay protocol (described below), modified to account for the character of the surfaces treated with a paint comprising OPH.

The form of OPH in the biomolecular composition contains both the changes of the previously described H254R mutant and the H257L mutant, and is corresponding designated the “H254R, H257L mutant.” The H254R, H257L mutant demonstrates a several-fold enhanced rates of R-VX catalysis relative to either the H254R mutant or the H257L mutant, and a 20-fold enhancement of activity relative to wild-type OPH. This version of the OPH biomolecular composition has been assayed in paints treated with soman or R-VX, and are described below.

Following standard protocols, OPD painted surfaces were uniformly contaminated with an isopropanol solution containing the chemical warfare agent soman. The concentration of soman on each contaminated surface was 1.0 mg/cm². The contaminated plates were maintained at or slightly above room temperature (≧20° C.) without any forced air-flow for various periods of time. A zero-time, 15 minutes, 30 minutes, and 45 minutes sample was taken for each control and biomolecular composition-containing plate series. To terminate the reaction and isolate residual soman on the plate surface, each plate was submerged in a container of isopropanol at the end-point and placed on a shaker to thoroughly extract any residual nerve agent. The solubilized portions were then quantified for soman. These assays showed that both the forms of OPH biomolecular composition were effective in detoxifying soman on metal surfaces. The two different OPH biomolecular compositions assayed detoxified the soman at levels over 65% and 77% after 45 minutes (Nato Army Armaments Group Project Group 31 on Non-Corrosive, Biotechnology-Based Decontaminants for CBW Agents, 2002). Additional assays with a CWA simulant indicated that had the NATO assay run for one to two hours, substantially all of the soman would have been detoxified.

### EXAMPLE 32

This Example relates to a demonstration of an OPH biomolecular composition at Aberdeen Proving Ground (SBCCOM) in Aberdeen, Md. In these assays, a primed wooden stick was coated with paint containing OPH biomolecular composition. The painted sticks used were 2 milimeter (“mm”) in diameter×15 mm in length. By estimating that the paint layer was 0.25 mm thick, the resulting surface area was approximately 125 mm². After coating the stick with paint containing OPH biomolecular composition and allowing the paint to dry, the coated stick was inserted into a microfuge tube containing 100 μl of 3.24 mM Russian-VX agent in saline and 900 μl phosphate buffer at pH 8.3. The tubes containing R-VX and the painted sticks were allowed to sit overnight in a hood at room temperature. Appropriate controls were run simultaneously.

The following morning, the contents of the microfuge tubes were assayed for free thiols by the ellman method. 10 mM DTNB [molecular weight (“MW”) 396.3] was prepared in 10 mM phosphate buffer at pH 8.0 for use as the indicator of enzyme activity. OPH paint's cleavage of R-VX releases a free thiol that reacts with DNTP to produce a colored product detectable spectrophotometrically at 405 nm. Ten μl of the microfuge tube contents, 100 μl DTNB solution and 890 μl phosphate buffer at pH 8.3 were read for thiol release at 405 nm using a Varian Carey 300 Spectrophotometer. The spectrophotometer was blanked with an unpainted stick control reaction. The molar equivalent of the R-VX hydrolyzed was determined using an extinction coefficient of 14,150 and the Beer-Lambert equation to calculate the product concentration. Results indicated that overnight exposure to OPH paint coated sticks resulted in decontamination of Russian VX from 32.4 μM in the original tube to less than 1 μM.

### EXAMPLE 33

The present Example relates to the NATO protocols for organophosphorus CWA decontamination, and describes a method for determining the decontamination properties of a coating, specifically paint, comprising a phosphoric triester hydrolase biomolecular composition. NATO assay requirements will be followed as closely as possible. Although actual assaying protocols among NATO countries vary somewhat, standard to all is the level of contamination. For exterior surfaces it is 10 grams per meter squared (“g/m²”). For interiors it is 1 g/m². Basic elements of NATO assaying procedures are as follows:

A. Coated Surface—A 10×10 cm metal plate coated with a coating that may comprise a biomolecular composition.

B. Contamination—Usually achieved with a multi-channel micropipette that can dispense 1 μl drops, with 100 drops per 10×10 cm metal plate.

C. Incubation—The plates will be placed into a sealed incubator, at 25° C. or 30° C., for a period ranging from 30 minutes to 3 hours.

D. Decontamination—The decontamination protocol varies according to the system being assayed. For example, spraying of decontamination solutions will last between 5 seconds to 20 seconds, depending on the pressure of the system.

E. Sampling—For standard solution-based decontamination, the assays will be normally prepared in a way that run-off decontaminant will be collected after it comes in contact with the plates and the CWA agent or CWA simulant. A set of plates will be removed for analysis at intervals, with the most common being 15 minutes and 30 minutes. Any residual liquid on the plates will be added to the run-off. For enzyme biomolecular composition assays, the plates will be not rinsed after decontamination, although the rinse is standard with other decontaminants. This rinsate would also be collected for analysis. A set of plates without decontamination will be used as 0 minute, 15 minute, and 30 minute controls.

F. Analysis—The run-off liquid and rinsate will be immediately extracted with a solvent, such as, for example, chloroform, hexane, etc., known to dissolve the CWA agent or CWA simulant. The plates themselves can be subjected to two types of analysis: contact hazard and off-gas hazard. For contact hazard, the plates will be covered with an absorbent material. For example, the French government uses silica gel TLC plates, and the government of the USA uses a dental dam as the absorbent material. In either case, the absorbent material is held in place with a weight and incubated for 15 minutes to 30 minutes at 25° C. or 30° C. The absorbent will be removed and extracted with solvent. The plates will be then extracted with solvent to determine residual agent absorbed into the coating, and thus the contact hazard. If surface decontamination efficiency, specifically the amount of residual agent detectable, is the variable being assessed, the plates will be immediately extracted with solvent, eliminating the contact hazard step. All of the solvent samples will be analyzed by Gas Chromatography (“GC”) with a flame photometric detector (“FPD”) and a phosphorus filter for nerve agents. Some countries use Gas Chromatography-Mass Spectrometry (“GC-MS”) for the analysis.

### EXAMPLE 34

This Example is of batch fermentation to produce OPH. Batch Culture-Rich Medium comprised 24 g/L yeast extract; 12 g/L casein hydrolysate; 4 ml/L glycerol; 2.31 g/L KH₂PO₄; 12.54 g/L K₂HPO₄; 0.24 g/L CoCl₂.6H₂O; 2 g/L glucose; 0.2 ml/L PPG2000; and 100 μg/ml ampicillin.

Batch Culture-5 L scale was grown at the following conditions: 30° C.; 400-450 rpm agitation; DO controlled at 20%; uncontrolled initial pH between 6.8-6.9; 5 Lpm (1 vvm) aeration; and atmospheric pressure. Over a time period of 0 to 50 hours, the Escherichia coli strain's growth was measured by optical density at 600 nm, the specific paraoxonase activity was determined (μmol ml⁻¹ min⁻¹), the volumetric paraoxonase activity was determined (μmol ml⁻¹ min⁻¹), the pH measured over a range of pH 6 to pH 9, the agitation measured over a range of 0 rpm to 500 rpm, and the dissolved oxygen measured over a range of 0% to 100%.

Batch Culture-400 L scale was grown at the following conditions: 30° C.; 150-200 rpm agitation; DO at 0-100%; uncontrolled initial pH 6.58; 200-300 Lpm (0.5-0.75 vvm) aeration; and tank pressure at 0-10 psi. Over a time period of 0 to 30 hours, the Escherichia coli strain's growth was measured by optical density at 600 nm, the specific paraoxonase activity was determined (μmol ml⁻¹ min⁻¹), the volumetric paraoxonase activity was determined (μmol ml⁻¹ min⁻¹), the pH measured over a range of pH 6 to pH 8, the agitation measured over a range of 0 rpm to 200 rpm, the dissolved oxygen measured over a range of 0% to 100%, the aeration rate measured over a range of 0 to 300 Lpm, and the tank pressure measured over a range of 0 psi to 12 psi.

### EXAMPLE 35

The following Example is of a large-scale fed-batch fermentation to produce OPH. Fed Batch Culture-Defined Medium comprised 13.3 g/L KH₂PO₄; 4 g/L (NH₄)₂SO₄; 1.7 g/L citric acid; 10 g/L glycerol; 1.2 g/L MgSO₄.7H₂O; 0.024 g/L MnCl₂.4H₂O; 2.26 mg/L CuCl₂.H₂O; 5 mg/L H₃BO₃; 4.5 mg/LThiamine.HCl; 4 mg/L Na₂MoO₄.7H₂O; 0.06 g/L Fe(III) citrate; 8.4 mg/L EDTA; 4 mg/L CoCl₂.6H₂O; 8 mg/L Zn(acetate)₂.H₂O; and 100 μg/ml ampicillin.

Feed: 500 g/L carbon source and 10 g/L MgSO₄.7H₂O. Batch Culture-5 L scale was grown at the following conditions: 30° C.; 200-1000 rpm agitation; DO controlled at 20%; pH controlled at 6.5; 5 Lpm (1 vvm) aeration; and atmospheric pressure. Feed was initiated as the 16^{th} hour, with the feed rate profile a constant rate with stepwise increments. Over a time period of 0 to 70 hours, the Escherichia coli strain's growth was measured by optical density at 600 nm, the specific paraoxonase activity was determined (μmol ml⁻¹ min⁻¹), the volumetric paraoxonase activity was determined (μmol ml⁻¹ min⁻¹), the pH measured over a range of pH 6 to pH 9, and the addition of the feed measured from 0 ml to 1000 ml.

### EXAMPLE 36

It is contemplated that any described coating composition may be altered (e.g., by direct addition and/or coating component substitution) to incorporate the biomolecular composition. The previous embodiments primarily described compositions and techniques for preparing, testing, and using a coating prepared de novo. However, it is contemplated that the biomolecular composition may be incorporated into a standard coating by direct addition, as described in Example 28. In specific aspects, it is contemplated that such added biomolecular composition may comprise 0.000001% to 85% or more, including all intermediate ranges and combinations thereof, by weight or volume, of the final composition produced by a combination of a coating and the biomolecular composition.

Alternatively, it is contemplated that a previously described coating composition may be altered by substitution (“replacement”) of one or more coating components, particularly a binder and/or a particulate material coating component (e.g., a pigment, a rheological control agent, a dispersant) by the biomolecular composition. It is contemplated that 0.000001% to 100%, including all intermediate ranges and combinations thereof, of the binder and/or particulate material coating component may be substituted by biomolecular composition. Additionally, the concentration of a biomolecular composition may exceed 100%, by weight or volume, of the substituted coating component. In specific aspects, a coating component may be substituted with a biomolecular composition equivalent to 0.000001% to 500%, including all intermediate ranges and combinations thereof, of the coating component. For example, a 20% (e.g., 2 kg) of a dispersant may be replaced by 10% (e.g., 1 kg) of the biomolecular composition to produce a coating with similar dispersion properties as a non-substituted formulation. In an additional example, 70% of a specific pigment (e.g., 7 kg) may be replaced by the equivalent of 127% (e.g., 12.7 kg) of the biomolecular composition to produce a coating with similar hiding power as a non-substituted formulation. The various assays described herein, or in the art in light of the present disclosures, may be used to determine the properties of a coating and/or film produced by direct addition and/or coating component substitution by the biomolecular composition.

The following is an example of an exterior gloss alkyd house paint that comprises various particulate materials (e.g., silica, a shading pigment, bentonite clay) that may incorporate a biomolecular composition. This example of an exterior gloss alkyd house paint comprises a grind and a letdown. The grind comprises by weight or volume: a first alkyd 232.02 lb or 29.9 gallons; a second alkyd 154.2 lb or 20 gallons; an aliphatic solvent (e.g., duodecane) 69.55 lb or 1.7 gallons; lecithin 7.8 lb or 0.91 gallons; TiO2 185.25 lb or 5.43 gallons; 10 micron silica 59.59 lb or 2.7 gallons; bentonite clay 18.00 lb or 1.44 gallons; a second alkyd 97.22 lb or 12.61 gallons; a first alkyd 69.84 lb or 9.00 gallons; and mildewcide 7.8 lb or 0.82 gallons. The letdown comprises by weight or volume: aliphatic solvent (e.g., dudecane) 19.50 lb or 3.00 gallons; a first drier (e.g., 12% solution cobalt) 2.00 lb or 0.23 gallons; a second drier (e.g., 18% solution Zr) 2.92 lb or 0.32 gallons; a third drier 3 (e.g., 10% solution Ca) 8.00 lb or 0.98 gallons; methyl ethyl ketoxime (Anti skinning agent) 3.22 lb or 0.42 gallons; an aliphatic solvent 9.75 lb or 1.50 gallons; and a shading pigment 0.3 lb or 0.04 gallons. In some embodiments, the particulate material of the coating formulation may be partly or fully substituted by the biomolecular composition. In other embodiments, the above formulation may be enhanced by direct addition of a biomolecular composition.

In another example, the following exterior flat latex house paint may be modified to incorporate a biomolecular composition. This example of an exterior flat latex house paint formulation, in typical order of addition, by weight or volume: water, 244.5 lb or 29.47 gallons; hydroxyethylcellulose, 3 lb or 0.34 gallons; glycols, 60 lb or 6.72 gallons; polyacrylate dispersant, 6.8 lb or 0.69 gallons; biocides, 10 lb or 1 gallons; non-ionic surfactant, 1 lb or 0.11 gallons; titanium dioxide, 225 lb or 6.75 gallons; silicate mineral, 160 lb or 7.38 gallons; calcined clay, 50 lb or 2.28 gallons; acrylic latex, @60%, 302.9 lb or 34.42 gallons; coalescent, 9.3 lb or 1.17 gallons; defoamers, 2 lb or 0.26 gallons; ammonium hydroxide, 2.2 lb or 0.29 gallons; 2.5% HEC solution, 76 lb or 9.12 gallons. In some embodiments, the particulate material (e.g., silicate mineral, calcined clay, titanium dioxide) of this coating formulation may be partly or fully substituted by the biomolecular composition. In other embodiments, the above formulation may be enhanced by direct addition of a biomolecular composition.

It is contemplated that any such previously described coating formulation may be modified to incorporate a biomolecular composition. Examples of described coating compositions include over 200 industrial water-borne coating formulations (e.g., air dry coatings, air dry or force air dry coatings, anti-skid of non-slip coatings, bake dry coatings, clear coatings, coil coatings, concrete coatings, dipping enamels, lacquers, primers, protective coatings, spray enamels, traffic and airfield coatings) described in “Industrial water-based paint formulations,” 1988, over 550 architectural water-borne coating formulations (e.g., exterior paints, exterior enamels, exterior coatings, interior paints, interior enamels, interior coatings, exterior/interior paints, exterior/interior enamels, exterior/interior primers, exterior/interior stains), described in “Water-based trade paint formulations,” 1988, the over 400 solvent borne coating formulations (e.g., exterior paints, exterior enamels, exterior coatings, exterior sealers, exterior fillers, exterior primers, interior paints, interior enamels, interior coatings, interior primers, exterior/interior paints, exterior/interior enamels, exterior/interior coatings, exterior/interior varnishes) described in “Solvent-based paint formulations,” 1977; and the over 1500 prepaint specialties and/or surface tolerant coatings (e.g., fillers, sealers, rust preventives, galvanizers, caulks, grouts, glazes, phosphatizers, corrosion inhibitors, neutralizers, graffiti removers, floor surfacers) described in Prepaint Specialties and Surface Tolerant Coatings, by Ernest W. Flick, Noyes Publications, 1991.

### EXAMPLE 37

To provide a description that is both concise and clear, various examples of ranges have been identified herein with the phrase “including all intermediate ranges and combinations thereof.” Examples of specific values (e.g., %, kD, ° C., μm, kg/L, Ku) that can be within a cited range by the reference to “including all intermediate ranges and combinations thereof” include 0.000001, 0.000002, 0.000003, 0.000004, 0.000005, 0.000006, 0.000007, 0.000008, 0.000009, 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.10, 99.20, 99.30, 99.40, 99.50, 99.60, 99.70, 99.80, 99.90, 99.91, 99.92, 99.93, 99.94, 99.95, 99.96, 99.97, 99.98, 99.99, 99.999, 99.9999, 99.99999, 99.999999, 99.9999999, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5250, 5500, 5750, 6000, 6250, 6500, 6750, 7000, 7250, 7500, 7750, 8000, 8250, 8500, 8750, 9000, 9250, 9500, 9750, 10,000, 25,000, 50,000, 75,000, 100,000, 250,000, 500,000, 1,000,000, or more. Additional examples of the use of this definition to specify sub-ranges are given herein.

In embodiments wherein a value or range is denoted in exponent form, both the integer and the exponent values are included in the definition of “including all intermediate ranges and combinations thereof.” For example, a range of 1.0×10⁻¹⁷ to 2.5×10⁻⁷, including all intermediate ranges and combinations thereof, would include a description for a sub-range such as 1.24×10⁻¹⁷ to 8.7×10⁻¹¹

However, general sub-ranges for each type of unit (e.g., %, kDa, ° C., μm, kg/L, Ku) are contemplated, as the values typically found within a particular type of unit are of a sub-range of the intergers described above. For example, integers typically found within a cited percentage range, as applicable, include 0.000001% to 100%, including all intermediate ranges and combinations thereof. Examples of values that can be within a cited molecular mass range in kilo Daltons (“kDa”) as applicable for many coating components include 0.50 kDa to 110 kDa, including all intermediate ranges and combinations thereof. Examples of values that can be within a cited temperature range in degrees Celsius (“° C.”) as is typically applicable in the arts of coatings and surface treatments include −10° C. to 500° C., including all intermediate ranges and combinations thereof. Examples of values that can be within a thickness range in micrometers (“μm”) as is typically applicable to coating and/or film thickness upon a surface include 1 μm to 2000 μm, including all intermediate ranges and combinations thereof. Examples of values that can be within a cited density range in kilograms per liter (“kg/L”) as is typically applicable in the arts of coatings and surface treatments include 0.50 kg/L to 20 kDa, including all intermediate ranges and combinations thereof. Examples of values that can be within a cited shear rate range in Krebs Units (“Ku”), as is typically applicable in the arts of coatings and surface treatments, include 20 Ku to 300 Ku, including all intermediate ranges and combinations thereof.

### EXAMPLE 38

It is contemplated that a biomolecular composition may also be incorporated into an elastomer. Elastomers (“rubbers”) are polymers that can undergo large, but reversible, deformations upon a relatively low physical stress. It is contemplated that an elastomer composition may incorporate a biomolecular composition, such as by preparation with the biomolecular composition and/or direct addition such as by a multi-pack composition. Elastomers (e.g., tire rubbers, polyurethane elastomers, polymers ending in an anionic diene, segmented polyerethane-urea copolymers, diene triblock polymers with styrene-alpha-methylstyrene copolymer end blocks, poly(p-methylstyrene-b-p-methylstyrene), polydimethylsiloxane-vinyl monomer block polymers, chemically modified natural rubber, polymers from hydrogenated polydienes, polyacrylic elastomers, polybutadienes, trans-polyisoprene, polyisobutene, cis-1,4-polybutadiene, polyolefin thermoplastic elastomers, block polymers, polyester thermoplastic elastomer, thermoplastic polyurethane elastomers) and techniques of elastomer synthesis and elastomer property analysis have been described, for example, in Walker, B. M., ed., Handbook of Thermoplastic Elastomers, Van Nostrand Reinhold Co., New York, 1979; Holden, G., ed., et. al., Thermoplastic Elastomers, 2^{nd} Ed., Hanser Publishers, Verlag, 1996.

### EXAMPLE 39

A filler is a bulk material in a composition. Extender pigments are used as a filler for coatings. In certain embodiments, a biomolecular composition may be used as a filler for various compositions. Examples of compositions that use fillers that are contemplated herein for incorporation of a biomolecular composition, include a composition comprising a polymer, thermoplastic material, a thermostat material, an elastomer, or a combination thereof. Such filler comprising materials have been described in Gerard, J. F., ed., Fillers and Filled Polymenrs-Macromolecular Symposia 169, Wiley-VCH, Verlag, 2001; Slusarski, L., ed., Fillers for the New Millenium-Macromolecular Symposia 194, Wiley-VCH, Verlag, 2003; and Landrock, A. H., Adhesives Technology Handbook, Noyes Publications, New Jersey, 1985.

### EXAMPLE 40

This Example relates to the use of adhesives and sealants. An adhesive is a composition that is capable of holding at least two surfaces together in a strong and permanent manner. A sealant is a composition capable of attaching to at least two surfaces, filling the space between them to provide a barrier or protective coating. In certain embodiments, a biomolecular composition may be used as a component of an adhesive or a sealant, such as, for example, by direct addition, substitution of an adhesive or sealant component (e.g., a particulate material), or a combination thereof.

Examples of adhesives and sealants (e.g., caulks, acrylics, elastomers, phenolic resin, epoxy, polyurethane, anarobic and structural acrylic, high-temperature polymers, water-based industrial type adhesives, water-based paper and packaging adhesives, water-based coatings, hot melt adhesives, hot melt coatings for paper and plastic, epoxy adhesives, plastisol compounds, construction adhesives, flocking adhesives, industrial adhesives, general purpose adhesives, pressure sensitive adhesives, sealants, mastics, urethanes,) for various surfaces (e.g., metal, plastic, textile, paper), adhesive and sealant components (e.g., antifoams, antioxidants, extenders, fillers, pigments, flame/fire retardants, oils, polymer emulsions, preservatives, bactericides, fungicides, resins, rheological/viscosity control agents, starches, waxes, acids, aluminum silicates, antiskinning agents, calcium carbonates, catalysts, cross-linking agents, curing agents, clays, corn starch, starch derivatives, defoamers, antifoams, dispersing agents, emulsifying agents, epoxy resin diluents, lattices, polybutenes, polyvinyl acetates, preservatives, acrylic resins, epoxy resins, ester gums, ethylene/vinyl acetate resins, maleic resins, natural resins, phenolic resins, polyamide resins, polyethylene resins, polypropylene resins, polyterpene resins, powder coating resins, radiation coating resins, urethane resins, vinyl chloride resins, emulsion resins, dispersion resins, resin esters, rosins, silicas, silicon dioxide, stabilizers, surfactants/surface active agents, talcs, thickeners, thixotropic agents, waxes) techniques of preparation and assays for properties, have been described in Skeist, I., ed., Handbook of Adhesives, 3^{rd} Ed., Van Nostrand Reinhold, N.Y., 1990; Satriana, M. J. Hot Melt Adhesives: Manufacture and Applications, Noyes Data Corporation, New Jersey, 1974; Petrie, E. M., Handbook of Adhesives and Sealants, McGraw-Hill, New York, 2000; Hartshorn, S. R., ed., Structural Adhesives-Chemistry and Technology. Plenum Press, New York, 1986; Flick, E. W., Adhesive and Sealant Compound Formulations, 2^{nd} Ed., Noyes Publications, New Jersey, 1984; Flick, E., Handbook of Raw Adhesives 2^{nd} Ed., Noyes Publications, New Jersey, 1989; Flick, E., Handbook of Raw Adhesives, Noyes Publications, New Jersey, 1982; Dunning, H. R., Pressure Sensitive Adhesives—Formulations and Technology, 2^{nd} Ed., Noyes Data Corporation, New Jersey, 1977; and Flick, E. W., Construction and Structural Adhesives and Sealants, Noyes Publications, New Jersey, 1988.

### EXAMPLE 41

This Example relates to the use of textiles. It is contemplated that a biomolecular composition may also be incorporated (e.g., direct addition to a formulation, incorporation as a component of a de novo formulation during preparation, etc.) into a material applied to a textile, such as, for example, a textile finish. Materials for application to a textile, textile finishes (e.g., soil-resistant finishes, stain-resistant finishes) and finish components (e.g., antioxidants, defoamers, antimicrobials, wetting agents, flame retardants, softeners, soil repellents, hand modifiers, antistatic agents, biocides, fixatives, scouring agents, dispersants, defoamers, anticracking agents, binders, stiffeners, cohesive agents, fiber lubricants, emulsifiers, antistats, yarn to hard surface lubricants) as well as assays for determining their properties are described, for example, in Johnson, K., Antistatic Compositions for Textiles and Plastics, Noyes Data Corporation, New Jersey, 1976; Rouette, H. K., Encyclopedia of Textile Finishing, Springer, Verlag, 2001; “Textile Finishing Chemicals: An Industrial Guide,” by Ernest W. Flick, Noyes Publications, 1990; “Handbook of Fiber Finish Technology,” by Philip E. Slade, Marcel Dekker, 1998; “ASTM Book of Standards, Volume 07.01 Textiles (I),” 2003; and “ASTM Book of Standards, Volume 07.02 Textiles (II),” 2003. A specific example of a textile finish is the trademark formulations of water repellent and/or oil repellent finish known as Scotchguard™ (3M Corporate Headquarters, Maplewood, Minn., U.S.A.).

### EXAMPLE 42

This Example relates to the use of a wax and wax related materials (e.g., a polish, a wax related cleaning material, etc.). It is contemplated that a biomolecular composition may also be incorporated (e.g., direct addition to a formulation, incorporation as a component of a de novo formulation during preparation, etc.) into a material (e.g., a wax, a polish, etc.) applied to a surface or impregnated into another material after manufacture. Waxes, polishes, floor coverings, cleaning materials, and related formulations (e.g., natural waxes, fossil waxes, earth waxes, peat waxes, montana waxes, lignite paraffins, petroleum waxes, synthetic waxes, commercial modified, blended, and compounded waxes, emulsifiable waxes, waxy alcohols, waxy acids, metallic soaps, compounded waxes, paraffin wax compounds, ethyl cellulose and wax mixtures, compositions with resins and rubber) and methods of preparation of waxes, polishes, floor coverings, cleaning materials, and related formulations and assays for their properties have been described, for example, in Warth, A. H., “The Chemistry and Technology of Waxes,” Reinhold Publishing Corporation, New York, 1956; Bennet, H., “Industrial Waxes Volume II Compounded Waxes and Technology,” Chemical Publishing Co., New York, 1975; “Industrial Waxes Volume I Natural & Synthetic Waxes,” Chemical Publishing Co., New York, 1975; Flick, E. W., “Advanced Cleaning Product Formulations Household, Industrial, Automotive,” 1989; Flick, E. W., “Institutional and Industrial Cleaning Product Formulations,” 1985; Flick, E. W., “Household and Automotive Chemical Specialties Recent Formulations,” 1979; Flick, E. W., “Household, Automotive, and Industrial Chemical Formulations 2^{nd} Edition,” 1984; Flick, E. W., “Household and Automotive Cleaners and Polishes 3^{rd} Edition,” 1986; “Ullmann's Encyclopedia of Industrial Chemistry, Volume 28,” 1996; “Coatings Technology Handbook 2^{nd} Edition Revised and Expanded,” 2001; Sequeira, A. Jr., “Lubricant Base Oil and Wax Processing,” 1994; “ASTM Book of Standards, Volume 15.04 Soaps and Other Detergents; Polishes; Leather; Resilient Floor Coverings,” 2003; “ASTM Book of Standards, Volume 05.01 Petroleum Products and Lubricants (I),” 2003; “ASTM Book of Standards, Volume 05.02 Petroleum Products and Lubricants (II),” 2003; and “ASTM Book of Standards, Volume 05.03 Petroleum Products and Lubricants (III),” 2003.

### EXAMPLE 43

This Example relates an additional embodiment where it is contemplated that the following organisms produce an OPAA that may be used in a biomolecular composition: Acinetobacter calcoaceticus ATCC 19606, Aeromonas hydrophila ATCC 7966, Aeromonas proteolytica, Arm. A isolate 1, Arm. A isolate 2, Bacillus subtilis (fr. Zuberer), Bacillus subtilis, ATCC 18685, Bacillus subtilis BRB41, Bacillus subtilis Q, Bacillus thuringensis (fr. Zuberer), Burkholderia cepacia LB400, Burkholderia cepacia T, Citrobacter diversus, Citrobacter freundii ATCC 8090, Edwardsiella tarda ATCC 15947, Enterobacter aerogenes ATCC 13048, Enterobacter cloacae 96-3, Enterobacter liquefaciens 363, Enterobacter liquefaciens 670, Erwinia carotovora EC189-67, Erwinia herbicola, Erwinia herbicola (agglomerans), Escherichia coli E63, Hafnia alvei ATCC 13337, Klebsiella pneumoniae ATCC 13883, Lactobacillus casei 686, Lactococcus lactis subsp. lactis pIL253, Proteus morganaii, Proteus vulgaris ATCC 13315, Pseudomonas aeriginosa ATCC 10145, Pseudomonas aeriginosa ATCC 27853, Pseudomonas flourescens, Pseudomonas putida ATCC 18633, Pseudomonas putida PpY101, Pseudomonas sp. P, Salmonella typhimurium ATCC 14028, Serratia marcescens ATCC 8100, Serratia marcescens HY, Serratia marcescens Nima, Shigella flexneri ATCC 12022, Shigella sonnei ATCC 25931, Staphylococcus aureus ATCC 25923, Staphylococcus sp. S, Streptococcus faecalis ATCC 19433, Vibrio parahaemolyticus TAMU 109, Yersinia enterocolitica ATCC 9610, Yersinia enterocolitica TAMU 84, Yersinia frederiksenii TAMU 91, Yersinia intermedia ATCC 29909, Yersinia intermedii TAMU 86, Yersinia kristensenia ATCC 33640, Yersinia kristensenia TAMU 95, Yersinia sp. ATCC 29912, Vibrio proteolyticus ATCC 15338, Thermus sp. ATCC 31674, Streptomyces cinnamonensis subsp. Proteolyticus ATCC 19893, Deinococcus proteolyticus ATCC 35074, Clostridium proteolyticum ATCC 49002, Aeromonas jandaei ATCC 49568, Aeromonas veronii biogroup sobria ATCC 9071, Pseudoaltermonas haloplanktis ATCC 23821, Xanthomonas campestris ATCC 33913, Pseudoalteromonas espejiana ATCC 27025, Shewanella putrefasciens ATCC 8071, Stenotrophomonas maltophilus ATCC 13637, Ochrobactrum anthropi ATCC 19286, Desulfovibrio vulgaris, or a combination thereof.

### EXAMPLE 44

This Example describes assay procedure for quantitative assessment of surface activity of a composition comprising a biomolecular composition using medicine sticks/dowels. The equipment used is a U.V. Spectrophotometer, a U.V. 1 cm pathlength cuvettes, 3 ml and 100 μl volume, and 1.5 ml eppendorf tubes. The reagents used include paraoxon (MW 275.21, ChemService cat#PS-610), 99% CHES (“2-[cyclohexylamino]ethanesulfonic acid”), (MW 207.3, Sigma cat #C-2880), and CoCL₂ 6H₂O (MW 237.9, Sigma cat #C-3169). 1 M CoCl₂, sterile, can be prepared as 23.79 g CoCl₂ per 100 ml ddH₂O that is filter sterilized or autoclaved. 200 mM CHES, pH 9.0, sterile can be prepared as 4.15 g+80 ml ddH₂O, pH to 9.0 with NaOH, where the total volume with ddH₂0 is 100 ml, and can be filter sterilized or autoclaved. The assay buffer is 20 mM CHES, pH 9.0, 50 μM CoCl₂.

In a 1.5 mL Eppendorf tube add: paraoxon to 1 mM (ex: 126 μl of 12 mM paraoxon) and assay buffer to 1.5 ml (ex: 1374 μl CHES buffer). Add a 5 mm length of treated stick to start the reaction, mix by inverting. Take 10 μl samples at 1 minute intervals, diluting with 90 μL CHES buffer into a 100 μl cuvette. Record the absorbance at 400 nm (A₄₀₀ₙₘ), blanking against CHES buffer+paraoxon. A small amount of hydrolysis of paraoxon without biomolecular composition may occur. Mix by inversion before each time point.

Alternatively, in a 3 ml cuvette, add: paraoxon to 1 mM (ex: 168 μl of 12 mM paraoxon), and assay buffer to 2.0 ml (ex: 1832 μl CHES buffer). Add a 5 (or 15 mm) length of treated stick to start the reaction. Record the (A₄₀₀ₙₘ) at the following time points: 0, 15, 30, 45, 60, 120, 180, 240, 300, 360, 420 and 480 minutes. Mix by inversion at regular intervals. If absorbencies above 2.5 are observed, dilute 10 μL samples with 90 μL CHES buffer in a 100 μL cuvette.

The following results demonstrate 90% degradation of the paraoxon over the time frame of measurement by a paroxonase bimolecular additive as determined by the dowel assay.

TABLE 86
Results Paroxonase Degradation

| Time | Replicates | | | | |
|---|---|---|---|---|---|
| (seconds) | A | B | C | umoles p-NP | Std Dev |
| 0 | 0.0218 | 0.0218 | 0.0224 | 0.0220 | 0.0003 |
| 120 | 0.1794 | 0.1518 | 0.1253 | 0.1522 | 0.0271 |
| 240 | 0.4359 | 0.3953 | 0.3418 | 0.3910 | 0.0472 |
| 360 | 0.7529 | 0.6541 | 0.6218 | 0.6763 | 0.0683 |
| 480 | 0.9494 | 0.8971 | 0.8894 | 0.9120 | 0.0327 |
| 600 | 0.9724 | 0.9688 | 0.9659 | 0.9690 | 0.0032 |
| 720 | 0.9706 | 0.9706 | 0.9729 | 0.9714 | 0.0014 |
| 840 | 0.9700 | 0.9694 | 0.9782 | 0.9725 | 0.0049 |
| 960 | 0.9535 | 0.9535 | 0.9435 | 0.9502 | 0.0058 |
| 1080 | 0.9600 | 0.9935 | 0.9912 | 0.9816 | 0.0187 |
| 1200 | 0.9500 | 0.9665 | 0.9682 | 0.9616 | 0.0101 |

p-NP = reaction product

### EXAMPLE 45

This Example demonstrates the production of a biomolecular composition by fed-batch fermentation at 200L scale manufacture.

The production timeline is as follows:

TABLE 87
Production Timeline

| Day | Time | Operation | Comments |
|---|---|---|---|
| 2-4 weeks | NA | Order supplies | Ensure that all reagents and |
| before day 1 | | | supplies are ready for use |
| 1-30 days | NA | Make trace | Make sufficient trace element |
| before day 1 | | element solutions | solutions for all fermentations and |
| | | | seeds |
| 2-5 days | NA | Plasmid | The transformation should be |
| before day | | transformation | completed with sufficient time for |
| | | into host strain | the agar plate to develop discrete |
| | | | colonies before it is needed to |
| | | | inoculate seed cultures. |
| 2-14 days | NA | Make shake | At least 2 × 50 ml and 2 × 1 L flasks |
| before day 1 | | flasks for seed | are required |
| | | cultures | |
| 2-14 days | NA | Make antibiotic | At least 2.5 ml of 10% antibiotic |
| before day 1 | | for seed cultures | solution is required |
| Day 1 | 09:00 | Pre-seed culture | Inoculation of pre-seed culture |
| | | | flasks |
| Day 1 | 18:00 | Seed culture | Inoculation of seed culture flasks |
| Day 1 | 10:00 | Fermentor set up | Prepare base medium and |
| | | | fermentor, sterilize |
| Day 1 | 11:00 | Prepare feed | Prepare and sterilize solutions. |
| | | solution and | After sterilization, store, add or |
| | | other additions | attach solutions as appropriate |
| Day 2 | 09:30 | Prepare for | Get fermentor and all peripheral |
| | | inoculation | items ready for inoculation |
| Day 2 | 10:00 | Inoculate | Add 2 L of inoculum to fermentor |
| | | Fermentor | |
| Day 3 | 10:00-20:00 | Start feed | Start nutrient feed when initial |
| | | | glucose has been exhausted |
| Days 3-5 | | Monitor | Adjust feed rates, add cobalt |
| | | fermentor | chloride |
| Day 4 | 14:00 | Set up filtration | Prepare filtration system for next |
| | | system | day |
| Day 5 | 09:00 | Harvest | Diafilter with water, then |
| | | | concentrate cells |
| Day 5 | 14:00 | Package and ship | Package the concentrated cells in |
| | | | 20 L carboys or a 30 gallon drum |
| | | | and ship to Aero-Instant |
| Day 5 | 15:00 | Cleaning | Clean fermentor and filter |
| Day 5 | 14:00 | OPD assays | Do paraoxonase assays on |
| | | | fermentation and harvest samples |

The reagents and supplies needed are as follows:

TABLE 88
Reagents and Supplies Required

| Chemical | Supplier | Amount | |
|---|---|---|---|
| Yeast extract | USB | 30 | g |
| Tryptone | Difco | 30 | g |
| NaCl | Baker | 30 | g |
| Ampicillin | USB | 30 | g |
| KH₂PO₄ | Baker | 2.2 | kg |
| (NH₄)₂SO₄ | Baker | 0.7 | kg |
| Citric acid | Baker | 0.3 | kg |
| Antifoam 204 | Sigma | 250 | ml |
| CoCl₂•6H₂O | Fisher/sigma | 100 | g |
| CuCl₂•H₂O | Baker | 1 | g |
| H₃BO₃ | Baker | 2 | g |
| Na₂MoO₄ | Baker | 2 | g |
| Fe(III)citrate | Aldrich | 25 | g |
| EDTA | Baker | 5 | g |
| Glucose | USB/Pfanstiehl | 3.5 | kg |
| MgSO₄•7H₂O | Baker | 0.7 | kg |
| Thiamine•HCl | Sigma | 35 | g |
| NH₄OH | Fisher | 10 | L |
| Glycerol | Fisher | 20 | L |
| Paraoxon | | | |

TABLE 89
Supplies

| Item | Supplier | Amount |
|---|---|---|
| Sterile loops | Fisher | 1 pack |
| Erlenmeyer flasks | Fisher | 3 × 250 ml; 2 × 2 L |
| Nalgene 250 ml filter | Fisher | 5 |
| housings | | |
| Nalgene 500 ml filter | Fisher | 2 |
| housings | | |
| Size 16 silicone tubing | Fisher | 1 reel |
| Size 25 silicone tubing | Fisher | 1 reel |
| 5 m² Optisep 11,000 PS | NCSRT | 2 |
| filters, 0.5 μm | | |

Plasmid Transformation into Host strain: Transformation Day 1, do as follows: Purified OPD-RL plasmid is stored at −20° C. in a bioexpression and fermentatation facility (“BFF”) BioXpress −20° C. freezer. Remove the relevant vial(s) and thaw. Transform into E. coli DH5a (Invitrogen). Add 2 μl of plasmids to 200 μl Invitrogen DH5a competent cells. Incubate cells on ice for 25 minutes. Heat shock the cells in a water bath at 42° C. for 30 seconds, then return to the ice for 2 minutes. Aseptically add 500 μl sterile SOB (SOB: 900 ml of distilled H₂O, 20 g Bacto Tryptone, 5 g Bacto Yeast Extract, 2 ml of 5M NaCl, 2.5 ml of 1M KCl, 10 ml of 1M MgCl₂, 10 ml of 1M MgSO₄, IL with distilled H₂O). Incubate for 60 minutes at 37° C. Plate 650 μl and 50 μl of the cells in SOB medium onto LB agar with ampicillin (100 μg/ml). Spread for single colonies and incubate at 37° C. overnight. Transformation Day 2, do as follows: Remove the plates from the incubator. Store at 4° C.

Seed Production: LB Medium for Seed cultures as follows: LB medium is made in standard batches. The recipe used is as follows: 10 g/L tryptone (Difco); 10 g/L NaCl (Baker); and 10 g/L yeast extract (Difco).

Day 1, at 09:00—pre-seed the culture growth as follows: At approximately 08.30, turn on the laminar flow hood, swab with ethanol, and switch on the UV light for 10 minutes. Select 2×250 ml LB flasks each containing 50 ml of LB medium. Record the batch and chemical lot numbers of the materials that are used. Aseptically add 50 μl of 10% ampicillin stock solution to each flask, and attach a copy of the recorded material information. At 09.00, aseptically pick several colonies from the plate and resuspend in sterile medium. Incubate the flasks at 30° C. and 250 rpm in a New Brunswick Scientific Series 15 incubator/shaker for 9 h.

Day 1, at 17:30, do as follows: Remove 10 μl of culture and check microscopically to confirm that there is no contamination. If the cultures pass the microscopic examination proceed to the next seed stage. Turn on the laminar flow hood, swab with ethanol, and switch on the UV light for 10 minutes. Select two 2L LB flasks each containing 1L of LB medium. Attach a copy of record of the batch number and chemical lot numbers of the materials used. Aseptically add 1 ml of 10% ampicillin stock solution to each flask. Attach a copy of a record of the batch number and chemical lot numbers to the materials used. At 18.00, aseptically transfer 10-20 ml of the 50 ml pre-seed culture to each of the 2L flasks. Incubate the flasks at 30° C. and 250 rpm in a New Brunswick Scientific Series 15 incubator/shaker overnight. Record all information regarding times and date of procedure, materials used, personnel conducting the work, and reaction conditions, and attach a copy to the other recorded information.

Fermentor set up was as follows: Production is done at 200L scale. The approximate volumes break down as follows: 160 L batch medium; 2 L seed cultures; 30-40 L feed solution; 5-7 L base addition; 1-3 L sample removal; to produce a total volume of about 200 L.

The fermentor used is a WB Moore, Inc. 250L stainless steel fermentor equipped with an Allen Bradley PLC controller. Temperature, pH, agitation, aeration, pressure and oxygen addition are controlled. Dissolved oxygen is measured and controlled by a sequential cascade of agitation rate, aeration rate, pressure, and oxygen supplementation.

Day 1, 10:00, Prepare the fermentor as follows: Calibrate the pH probe. Check the DO probe. Replace the electrolyte and membrane if necessary. Insert the pH probe and DO probes. Add approximately 100L of DI water to the tank. Prepare the base medium. The following components are added to the fermentor prior to sterilization.

TABLE 90
Materials to be Added to Fermentor

| Chemical | Manufacturer | Amount required | |
|---|---|---|---|
| KH₂PO₄ | Baker | 2128 | g |
| (NH₄)₂SO₄ | Baker | 640 | g |
| Citric acid | Baker | 272 | g |
| Trace element | BFF | 160 | ml |
| solution A | | | |
| Trace element | BFF | 1600 | ml |
| solution B | | | |
| Antifoam 204 | Sigma | 20 | ml |
| Water | | QS to 155 | L |

Sterilize the tank at 122° C. for one hour. Cool the tank to 30° C. and set the control temperature. Record all information regarding times and date of procedure, materials used, personnel conducting the work, and reaction conditions, and attach a copy to the other recorded information. Prepare the medium additions as follows.

TABLE 91
Trace Element Solution A

| Chemical | Manufacturer | Amount required | |
|---|---|---|---|
| Citric acid | Baker | 2.5 | g |
| CoCl₂•6H₂O | Fisher/sigma | 1.0 | g |
| CuCl₂•H₂O | Baker | 0.57 | g |
| H₃BO₃ | Baker | 1.25 | g |
| Na₂MoO₄ | Baker | 1.0 | g |
| DI water | | QS to 500 | ml |

Store at 4° C. until use. Record all information regarding times and date of procedure, materials used, personnel conducting the work, and reaction conditions, and attach a copy to the other recorded information.

TABLE 92
Trace Element Solution B

| Chemical | Manufacturer | Amount required | |
|---|---|---|---|
| Fe(III) citrate | Aldrich | 24 | g |
| EDTA | Baker | 3.36 | g |
| DI water | | QS to 4 | L |

Store at 4° C. until use. Record all information regarding times and date of procedure, materials used, personnel conducting the work, and reaction conditions, and attach a copy to the other recorded information.

TABLE 93
Glucose Addition Solution

| Chemical | Manufacturer | Amount required | |
|---|---|---|---|
| Glucose | USB/Pfanstiehl | 3200 | g |
| MgSO₄•7H₂O | Baker | 192 | g |
| DI water | | QS to 6 | L |

Sterilize in an autoclave at 122° C. for one hour.

TABLE 94
Cobalt Chloride Solution

| Chemical | Manufacturer | Amount required | |
|---|---|---|---|
| CoCl₂ | Fisher/Sigma | 54.9 | g |
| DI water | | QS to 500 | ml |

Filter sterilize in two 250 ml aliquots using Nalgene 0.22 μm filter units.

TABLE 95
Thiamine Solution

| Chemical | Manufacturer | Amount required | |
|---|---|---|---|
| Thiamine•HCl | Sigma | 33.7 | g |
| DI water | | QS to 100 | ml |

Filter sterilize in using a Nalgene 0.22 μm filter unit.

Note: 2 ml of this solution will be added to the fermentor.

TABLE 96
Ampicillin Solution

| Chemical | Manufacturer | Amount required | |
|---|---|---|---|
| Ampicillin, sodium salt | USB | 20 | g |
| DI water | | QS to 250 | ml |

Filter sterilize in using a Nalgene 0.22 μm filter unit.

TABLE 97
Base Solution

| Chemical | Amount required |
|---|---|
| Aqueous NH₄OH | 7.5 L |

Sterilize an empty reservoir bottle at 122° C. for 30 minutes. When cool, empty three 2.5 L ammonium hydroxide bottles into the reservoir. Use extreme caution and wear protective clothing.

TABLE 98
Feed Solution

| Chemical | Amount required | |
|---|---|---|
| Glycerol | 20 | L |
| MgSO₄•7H₂O | 400 | g |
| DI water | QS to 40 | L |

Make up in reservoir tank fitted out for feeding the fermentor, with silicone tubing capable of feed rates of 2-40 ml/min. Sterilize the tank at 122° C. for one hour.

Fermentor Operations on, Day 2, 09:30, include: making additions to the Fermentor, adding the following solutions, in order:

TABLE 99
Fermentor Solution

| Addition | Amount | |
|---|---|---|
| Ampicillin solution | 250 | ml |
| Glucose/MgSO4 solution | 6 | L |
| Thiamine solution | 2 | ml |
| Cobalt chloride solution | 250 | ml |

With the feed bottle on the balance of the Scilog pump system, attach the feed reservoir to the feed port on the fermentor. Run the tubing through the scilog pump and prime the lines. With the base reservoir on the Ohaus balance, attach to the base port on the fermentor. Run the tubing through a peristaltic pump and prime the lines. Plug the pump into the base socket on the rear of the fermentor. Take a sample from the fermentor. Store a portion in a labeled sterile falcon tube. Check the pH of another portion offline. Adjust the pH calibration if necessary. Calibrate the dissolved oxygen probe. Check and set all fermentation parameters.

TABLE 100
Fermentation Parameters

| Parameter | Set point |
|---|---|
| Temperature | 30° C. |
| pH | 6.5 |
| Dissolved oxygen | 60 mBar (30%) |
| Air flow rate | 50-200 LPM |
| Agitation Rate | 100-350 rpm |
| Oxygen flow rate | 50 LPM (on demand) |
| Tank pressure | 0-5 psi |

Remove the seed culture flasks from the shaker and take 10 μl of culture to check microscopically to confirm that there is no contamination. Also check the OD₆₀₀ of the cultures. If the cultures pass the microscopic examination proceed to the next seed stage. Record all information regarding times and date of procedure, materials used, personnel conducting the work, and reaction conditions, and attach a copy to the other recorded information.

Day 2 10:00, inoculation, do as follows: Add the entire contents of the two seed culture flasks to the 250 L fermentor. From the harvest port, take a 20-50 ml sample. Measure the optical density at 600 nm. Using a Boehringer glucose analyzer, measure the glucose concentration of the medium. Read from the controller on the fermentor and the attached balances. Record all information regarding times and date of procedure, materials used, personnel conducting the work, and reaction conditions, and attach a copy to the other recorded information. Every 2-4 hours, take samples and process as described above. Record all information regarding times and date of procedure, materials used, personnel conducting the work, and reaction conditions, and attach a copy to the other recorded information.

Days 3-5, Start Feed as follows: When the glucose level is below 2 g/L start the feed pump. The glucose should be reduced to this level at between 24-36 hours after inoculation. At this point the sampling frequency may be reduced to 3-5 times per day.

Feed Profile is a follows: Program the following feed profile into the Scilog pump. Execute the program at the start of feeding.

TABLE 101
Feed Profile.

| | | Cumulative feed |
|---|---|---|
| Time | Feed Rate (ml/min) | added (L) |
| Feed start | 4 | 0 |
| Feed start + 2 h | 6 | 0.48 |
| Feed start + 4 h | 8 | 1.20 |
| Feed start + 6 h | 10 | 2.16 |
| Feed start + 8 h | 12 | 3.36 |
| Feed start + 10 h | 16 | 4.80 |
| Feed start + 36.67 h | 0 | 40.00 |

Samples for paraoxonase assays are as follows: From this point in the fermentation, when samples are taken, centrifuge 2×1 ml samples in eppendorf tubes and store the cells at −80° C. until testing for paraoxonase activity.

Cobalt chloride addition is as follows: When the OD₆₀₀ attains a level of 40±10, add the remaining cobalt chloride.

Fermentation Completion is as follows: The fermentation is complete when (1) the cells stop growing, as indicated by a combination of a drop in OD₆₀₀, a drop in oxygen demand and an increase in pH; (2) the feed is exhausted; (3) the elapsed fermentation time reaches 72 h. At the completion of the fermentation, turn off the feed pump and the base pump. Cool the reactor to <15° C. Note the condition of the culture at this time, as foaming is sometimes observed as the culture stops growing and is cooled. Take one or more sample from the fermentor and measure the average wet weight of the culture

Harvesting, Day 4, 14:00 is as follows: Set up the NCSRT filtration system. Use two 5m² Optisep 11,000 polysulfone filters, 0.05 μm pore size, 0.875 mm channel height. Rinse the system with at least 200 L of DI water.

Day 5, 08:00 is as follows: Fill a reservoir tank with 600 L of DI water. When the fermentation is complete and the culture has been cooled to <15° C., hook up the filtration system to the tank as follows: Release pressure from the tank and stop agitation. Attach the pump inlet to the fermentor drain. Place the filtration system return in the top of the fermentor. Connect the water reservoir to the feed inlet. Open the fermentor drain valve. Attach a line to the sample port to estimate culture volume. Estimate and record culture volume. Estimate and record cell mass in the fermentor. Keep a sample for paraoxonase assay.

Start filtration as follows: Start the filtration system pump at a low flow rate. As the system is filled, gradually increase the pump rate until the flow rate across the membrane is 300 L/min, or until the pressure at the bottom of the membrane is 10 psi, whichever comes first. Do not allow the membrane pressure to exceed 11 psi. Record all information regarding times and date of procedure, materials used, filtration data, personnel conducting the work, and reaction conditions, and attach a copy to the other recorded information. Measure and record the initial flux rate (L/min). Check that the filtrate is clear and that product is not crossing the membrane. If the filtrate is slightly cloudy reduce the flow rate and then recheck. Start adding DI water to the fermentor at a rate equal to the flux rate to maintain the culture volume. Diafilter with three volumes (600 L) of DI water, note the time at which diafiltration is complete.

When diafiltration is complete, continue filtering as before, to concentrate the washed culture. Monitor the membrane pressure, and reduce the pump rate is the pressure rises. Continue concentration until the cell density attains a level of 700±100 g/L or until the pump rate is too low to continue. Without shutting off the pump, open the system drain line and pump the product into 20L carboys. Take one or more sample of the final product and measure the wet weight, and average the wet weight. Measure the final product volume, and estimate the cell mass in product. Save a sample for a paraoxonase assay. Label the carboys and store at 4° C. ready for shipping.

Downstream Processing is as follows: The product is ready for spray drying applications. It may be shipped to other facilities on 20L carboys can be shipped with ice packs.

Cleaning is as follows: Clean the fermentor and filter system thoroughly.

The paraoxonase assay is as follows: This describes assaying of biomolecular composition for paraoxonase activity in a 96-well plate using a plate reader. The equipment and reagents used are shown on the table below.

TABLE 102
Equipment and Reagents

Equipment
Plate Reader
Reagents
Paraoxon (MW 275.21, ChemService cat#PS-610)
CHES (2-[cyclohexylamino]ethanesulfonic acid), 99% (MW 207.3, Sigma
cat# C-2880)

Sample preparation is as follows: paraoxon is prepared in the disclosures herein or by the techniques of the art; 200 mM CHES, pH 9.0, sterile is prepared by adding 4.15 g and 80 mL ddH₂O, adjusting to pH 9.0 with NaOH, bringing to 100 mL total volume with ddH₂O, and filter sterilizing or autoclaving; and working solutions prepared by diluting 200 mM CHES to 20 mM and 40 mM.

Plate Reader Assay is as follows: weighing approximately 15 mg of wet cell biomass (or dried additive) in a 1.5 mL Eppendorf tube; resuspending in appropriate volume 20 mM CHES to make 30 mg/mL suspension; prepare a serial dilution of this solution as 1:2, 1:5, and 1:10; loading 2 uL of each dilution in triplicate in the 96-well plate (i.e., wells 1-3 will have undiluted solution, 4-6 will all have 1:2, 7-9 will be 1:5 and 10-12 will be 1:10); adding 39.36 uL MilliQ H₂O to each of the wells; adding 50 uL 40 mM CHES to each well; adding 10.64 uL of 9.4 mM Paraoxon is added to each well; setting the kinetic protocol to read absorbance at 405 nm taking 50 readings, at 7 second intervals; and determining maximum velocity for analysis using usually at least 20 points.

Record personnel involved in the procedures implemented. Quality control and safety procedures were as described in Example 5, including use of a hood for material handling as needed.

### EXAMPLE 46

This Example demonstrates the harvesting of a biomolecular composition produced by fermentation.

Harvesting is as follows: Set up the NCSRT filtration system. Use two 5 m² Optisep 11,000 polysulfone filters, 0.05 μm pore size, 0.875 mm channel height. Rinse the system with at least 200 L of DI water. Fill a reservoir tank with 600 L of 100 mM sodium bicarbonate.

When the fermentation is complete and the culture has been cooled to <15° C., hook up the filtration system to the tank as follows: release pressure from the tank and stop agitation. Attach the pump inlet to the fermentor drain. Place the filtration system return in the top of the fermentor. Connect the water reservoir to the feed inlet. Open the fermentor drain valve. Attach a line to the sample port to estimate culture volume, and estimate the culture volume, cell mass in the fermentor, and keep a sample for the paraoxonase assay.

Start filtration as follows: Start the filtration system pump at a low flow rate. As the system is filled, gradually increase the pump rate until the flow rate across the membrane is 300 L/min, or until the pressure at the bottom of the membrane is 10 psi, whichever comes first. Do not allow the membrane pressure to exceed 11 psi. Record all information regarding times and date of procedure, materials used, filtration data, personnel conducting the work, and reaction conditions, and attach a copy to the other recorded information. Measure the initial flux rate (L/min). Check that the filtrate is clear and that product is not crossing the membrane. If the filtrate is slightly cloudy reduce the flow rate and then recheck. Start adding 100 mM sodium bicarbonate to the fermentor at a rate equal to the flux rate to maintain the culture volume. Diafilter with three volumes (600 L) of 100 mM sodium bicarbonate, and record the time at which diafiltration is complete.

When diafiltration is complete, continue filtering as before, to concentrate the washed culture. Monitor the membrane pressure, and reduce the pump rate is the pressure rises. Continue concentration until the cell density attains a level of 700±100 g/L or until the pump rate is too low to continue. Without shutting off the pump, open the system drain line and pump the product into 20 L carboys. Take one or more sample of the final product and measure the wet weight, and determine the average wet weight, measure the final product volume, estimate the cell mass in the product, and keep a sample for a paraoxonase assay. Label carboys and store at 4° C. ready for shipping to other facities or end users.

### EXAMPLE 47

This Example demsonstrates the preparation and chararcterization of the organophosphourus compound and OPH substrate, paraoxon for use in various other examples and assays described herein.

The equipment used is as follows: a U.V. Spectrophotometer, U.V. 1 cm pathlength cuvettes, and a stir plate.

The reagents used are as follows: Paraoxon, 200 mg (Chem Service, cat #PS-610, MW 275.21, [symbol] =8.9×10³)

Samples are prepared as follows: add 200 mgs of paraoxon, which should be as an oily liquid in 100 mg aliquots, to 50 mls ddH₂O; and letting stir in the cold for 2-3 days to be sure it is fully dispersed and dissolved, though as the paraoxon should be 14.5 mM; due to loss during pipetting, solubility, etc., the solution rarely reaches this concentration.

The analysis of samples should be conducted as follows: To determine the [paraoxon], make the following dilutions—1:100 with 10 μl paraoxon stock:990 μl ddH₂O, 1:500 with 2 μl paraoxon stock:998 μl ddH₂O, and 1:1000 with 10 μl (1:100) paraoxon:990 μl ddH₂O; read O.D. at 274 nm; with typical readings being—1:100=1, 1:500=0, and 1:1000=0. The extinction coefficient of diethyl p-nitrophenyl phosphate (paraoxon) is 8,900 M⁻¹ cm⁻¹, and the sample calculations are as follows: (1.1/8,900)*100=0.0123 μmol/μl*(0.0123 μmol/μl)*(1,000,000 μl/l)*(mm/1000 μmoles)=12.3 mM concentration of paraoxon

Procedural cautions: Make sure pipet tips fit the pipette. Check the liquid level in the tips for air bubbles, etc., particularly when using the multichannel pipettes. Quality control and safety procedures were as described in Example 5. Quality control included operating, maintaining, and maintanence of all equipment in accordance with normal practice of the art and any manuals provided from the manufacturer, and maintanence records kept; using correctly labeled working solutions prior to the date of expiration, and disposing of others which are out of date or prepared incorrectly; and disposing of leftover QC samples in the appropriate hazard container, and not using QC samples made one day on the next day.

### EXAMPLE 48

This Example relates to the use of a polymeric material. It is contemplated that a biomolecular composition (e.g., an enzyme) may also be incorporated (e.g., direct addition to a formulation, incorporation as a component of a de novo formulation during preparation, post preparation absorption, etc.) into a polymeric material. A polymeric material may comprise a plurality of polymers (“polymer blends”), an ionomer, a thermoplastic polymer, a thermoset polymer, or an elastomer. A laminate or a composite material may comprise a polymer (e.g., a thermoplastic polymer, a thermoset polymer, an elastomeric polymer). A thermoplastic comprises a thermoplastic polymer, while a thermoset plastic comprises a thermosetting polymer. A thermoplastic polymer may comprise an environmentally degradable polymers (e.g., a biodegradable polymer), a natural polymer, a photodegradable polymer, a synthetic biodegradable polymer (e.g., a poly(alkylene oxalate)s, a polyamino acid, a pseudo-polyamino acid, a polyanhydride, a polycaprolactone, a polycyanoacrylate, a polydioxanone, a polyglycolide, poly(hexamethylene-co-trans-1,4-cyclohexane dimethylene oxalate), a polyhydroxybutyrate, a polyhydroxyvalerate, a polylactide, a poly(ortho ester), a poly (p-dioxanone), a polyphosphazene, a poly(propylene fumarate), a polyvinyl alcohol), a biological degradable polymer (e.g., a collagen, a fibrinogen/fibrin, a gelatin, a polysaccharide), a cellulosic polymer (e.g., cellulose acetate, a cellulose acetate butyrate, a cellulose acetate propionate, a cellulose methylcellulose, a methylcellulose, a cellulosehydroxyethyl, an ethylcellulose, a hydroxypropylcellulose), a fluoropolymer, an ethylene chlorotrifluoroethylene, an ethylene tetrafluoroethylene, a fluoridated ethylene propylene, a polyvinylidene fluoride, a polychlorotrifluoroethylene, a polytetrafluoroethylene, a polyvinyl fluoride), a polyoxymethylene, a polyamide, an aromatic polyamide, a polyacrylonitrile, a polyamide-imide, a polyarylate, a polybenzimidazole, a polybutylene, a polycarbonate, a polyester (e.g., a liquid crystal polyester polycarbonate, a polybutylene terephthalate, a polycyclohexylenedimethylene terephthalate, a poly(ethylene terephthalate)), a polyetherimide, polyethylene (e.g., a very low-density polyethylene, a low-density polyethylene, a linear low-density polyethylene, a medium-density polyethylene, a high-density polyethylene, an ultrahigh molecular weight polyethylene, a chlorinated polyethylene, a chlorosulfonated polyethylene, a phosphorylated polyethylene, an ethylene-acrylic acid copolymer, an ethylene-methyl acrylate copolymer, an ethylene-ethyl acrylate copolymer, an ethylene-n-butyl acrylate copolymer, an ethylene-vinyl acetate copolymer, an ethylene-vinyl alcohol copolymer), a polyimide, a polyketone, a poly(methylmethacrylate), a polymethylpentene, a polyphenylene oxide, a polyphenol sulfide, a polyphthalamide, a polypropylene, a polyurethane, a polystyrene (e.g., styrene-acrylonitrile copolymer, a styrene-butadiene copolymer, an acrylonitrile butadiene styrene terpolymer, an acrylonitrile-chlorinated polyethylene-styrene terpolymer, an acrylic styrene acrylonitrile terpolymer), a polysulfone resin (e.g., a polysulfone, a polyaryl sulfone, a polyether sulfone), a polyvinyl chloride (e.g., a chlorinated polyvinyl chloride), a polyvinylidene chloride, or a combination thereof. A thermoset plastic may comprise, for example, an allyl resin, an amino resin, a bismaleimide resin, an epoxy resin, a phenolic resin, a polyester resin, a polyimide resin, a polyurethane resin, a silicon resin, or a combination thereof. Polymeric materials often comprise an additive, for example, a filler, a plasticizer, a lubricant, a flame retarder, a colorant, a blowing agent, an anti-aging additive, a cross-linking agent, or a combination thereof. Polymeric materials and methods of preparation of preparing a polymeric material and assays for a polymeric material's properties have been described, for example, “Handbook of Plastics, Elastomers, & Composites Fourth Edition” (Harper, C. A. Ed.) McGraw-Hill Companies, Inc, New York, 2002; and Tadmor, Z. and Costas, G. G. “Principles of Polymer Processing Second Edition,” John Wiley & Sons, Inc. Hoboken, N.J., 2006.

What is claimed:

1. A coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises an antimicrobial enzyme, a peptidase, an esterase, a petroleum lipolytic enzyme, or a combination thereof.

2. The coating of claim 1, wherein the antimicrobial enzyme comprises a lysozyme, a lysostaphin, a libiase, a lysyl endopeptidase, a mutanolysin, a cellulase, a chitinase, an α-agarase, an β-agarase, an N-acetylmuramoyl-L-alanine amidase, a lytic transglycosylase, a glucan endo-1,3-β-D-glucosidase, an endo-1,3(4)-β-glucanase, a β-lytic metalloendopeptidase, a 3-deoxy-2-octulosonidase, a peptide-N4-(N-acetyl-β-glucosaminyl)asparagine amidase, a mannosyl-glycoprotein endo-β-N-acetylglucosaminidase, a ι-carrageenase, a κ-carrageenase, a λ-carrageenase, an α-neoagaro-oligosaccharide hydrolase, an endolysin, an autolysin, a mannoprotein protease, a glucanase, a mannase, a zymolase, a lyticase. a lipolytic enzyme, or a combination thereof.

3. The coating of claim 1, wherein the antimicrobial enzyme comprises a lysozyme.

4. The coating of claim 1, wherein the antimicrobial enzyme comprises a lysostaphin.

5. The coating of claim 1, wherein the antimicrobial enzyme comprises a libiase.

6. The coating of claim 1, wherein the antimicrobial enzyme comprises a lysyl endopeptidase.

7. The coating of claim 1, wherein the antimicrobial enzyme comprises a mutanolysin.

8. The coating of claim 1, wherein the antimicrobial enzyme comprises a cellulase.

9. The coating of claim 8, wherein the cellulase comprises a α-cellulase.

10. The coating of claim 8, wherein the cellulase comprises a β-cellulase.

11. The coating of claim 1, wherein the antimicrobial enzyme comprises a chitinase.

12. The coating of claim 1, wherein the antimicrobial enzyme comprises an α-agarase.

13. The coating of claim 1, wherein the antimicrobial enzyme comprises an β-agarase.

14. The coating of claim 1, wherein the antimicrobial enzyme comprises an N-acetylmuramoyl-L-alanine amidase.

15. The coating of claim 1, wherein the antimicrobial enzyme comprises a lytic transglycosylase.

16. The coating of claim 1, wherein the antimicrobial enzyme comprises a glucan endo-1,3-β-D-glucosidase.

17. The coating of claim 1, wherein the antimicrobial enzyme comprises an endo-1,3(4)-β-glucanase.

18. The coating of claim 1, wherein the antimicrobial enzyme comprises a β-lytic metalloendopeptidase.

19. The coating of claim 1, wherein the antimicrobial enzyme comprises a 3-deoxy-2-octulosonidase.

20. The coating of claim 1, wherein the antimicrobial enzyme comprises a peptide-N4-(N-acetyl-β-glucosaminyl)asparagine amidase.

21. The coating of claim 1, wherein the antimicrobial enzyme comprises a mannosyl-glycoprotein endo-β-N-acetylglucosaminidase.

22. The coating of claim 1, wherein the antimicrobial enzyme comprises a ι-carrageenase.

23. The coating of claim 1, wherein the antimicrobial enzyme comprises a κ-carrageenase.

24. The coating of claim 1, wherein the antimicrobial enzyme comprises a λ-carrageenase.

25. The coating of claim 1, wherein the antimicrobial enzyme comprises an α-neoagaro-oligosaccharide hydrolase.

26. The coating of claim 1, wherein the antimicrobial enzyme comprises an endolysin.

27. The coating of claim 1, wherein the antimicrobial enzyme comprises an autolysin.

28. The coating of claim 1, wherein the antimicrobial enzyme comprises a mannoprotein protease.

29. The coating of claim 1, wherein the antimicrobial enzyme comprises a glucanase.

30. The coating of claim 1, wherein the antimicrobial enzyme comprises a mannase.

31. The coating of claim 1, wherein the antimicrobial enzyme comprises a zymolase.

32. The coating of claim 1, wherein the antimicrobial enzyme comprises a lyticase.

33. The coating of claim 1, wherein the antimicrobial enzyme comprises a lipolytic enzyme.

34. The coating of claim 33, wherein the lipolytic enzyme comprises a phospholipase.

35. The composition of claim 1, wherein the antimicrobial enzyme catalyzes a reaction that degrades a cell wall or cell membrane.

36. The composition of claim 1, wherein the esterase comprises a lipolytic enzyme, a sulfuric ester hydrolase, phosphoric triester hydrolase, or a combination thereof.

37. The composition of claim 1, wherein the peptidase possessing esterase activity.

38. The composition of claim 1, wherein the active enzyme comprises a plurality of active enzymes.

39. The composition of claim 1, wherein the coating comprises an interior coating.

40. The composition of claim 36, wherein the lipolytic enzyme comprises a carboxylesterase, a lipase, a lipoprotein lipase, an acylglycerol lipase, a hormone-sensitive lipase, a phospholipase A₁, a phospholipases A₂, a phosphatidylinositol deacylase, a phospholipase C, a phospholipase D, a phosphoinositide phospholipase C, a phosphatidate phosphatase, a lysophospholipase, a sterol esterase, a galactolipase, a sphingomyelin phosphodiesterase, a sphingomyelin phosphodiesterases D, a ceramidase, a wax-ester hydrolase, a fatty-acyl-ethyl-ester synthase, a retinyl-palmitate esterase, a 11-cis-retinyl-palmitate hydrolase, an all-trans-retinyl-palmitate hydrolase, a cutinase, an acyloxyacyl hydrolase, or a combination thereof.

41. The composition of claim 40, wherein the lipolytic enzyme comprises a combination of lipolytic enzymes.

42. The composition of claim 40, wherein the lipolytic enzyme comprises a carboxylesterase derived from Actinidia deliciosa, Aedes aegypti, Aeropyrum pernix, Alicyclobacillus acidocaldarius, Aphis gossypii, Arabidopsis thaliana, Archaeoglobus fulgidus, Aspergillus clavatus, Athalia rosae, Bacillus acidocaldarius, Bombyx mandarina, Bombyx mori, Bos taurus, Burkholderia gladioli, Caenorhabditis elegans, Canis familiaris, Cavia porcellus, Chloroflexus aurantiacus, Felis catus, Fervidobacterium nodosum, Helicoverpa armigera, Homo sapiens, Macaca fascicularis, Malus pumila, Mesocricetus auratus, Mus musculus, Musca domestica, Mycoplasma hyopneumoniae, Myxococcus xanthus, Neosartorya fischeri, Oryctolagus cuniculus, Paeonia suffruticosa, Pseudomonas aeruginosa, Rattus norvegicus, Rubrobacter xylanophilus, Spodoptera exigua, Spodoptera litura, Sulfolobus acidocaldarius, Sulfolobus shibatae, Sulfolobus solfataricus, Sus scrofa, Thermotoga maritime, Thermus thermophilus, Vaccinium corymbosum, Vibrio harveyi, Xenopsylla cheopis, Yarrowia lipolytica, or a combination thereof.

43. The composition of claim 42, wherein the lipolytic enzyme comprises a thermophilic carboxylesterase derived from Aeropyrum pernix, Alicyclobacillus acidocaldarius, Archaeoglobus fulgidus, Bacillus acidocaldarius, Pseudomonas aeruginosa, Sulfolobus shibatae, Sulfolobus solfataricus, Thermotoga maritime, or a combination thereof.

44. The composition of claim 36, wherein the lipolytic enzyme comprises a lipase derived from Acinetobacter, Aedes aegypti, Anguillajaponica, Antrodia cinnamomea, Arabidopsis rosette, Arabidopsis thaliana, Arxula adeninivorans, Aspergillus niger, Aspergillus oryzae, Aspergillus tamarii, Aureobasidium pullulans, Avena sativa, Bacillus licheniformis, Bacillus sphaericus, Bacillus stearothermophilus, Bacillus subtilis, Bacillus thermocatenulatus, Bacillus thermoleovorans, Bombyx mandarina, Bombyx mori, Bos Taurus, Brassica napus, Brassica rapa, Burkholderia cepacia, Caenorhabditis elegans, Candida albicans, Candida antarctica, Candida deformans, Candida parapsilosis, Candida rugosa, Candida thermophila, Canis domesticus, Chenopodium rubrum, Clostridium beijerinckii, Clostridium botulinum, Clostridium novyi, Danio rerio, Galactomyces geotrichum, Gallus gallus, Geobacillus, Gibberella zeae, Gossypium hirsutum, Homo sapiens, Kurtzmanomyces sp., Leishmania infantum, Lycopersicon esculentum L, Malassezia furfur, Methanosarcina acetivorans, Mus musculus, Mus spretus, Mycobacterium tuberculosis, Mycoplasma hyopneumoniae, Myxococcus xanthus, Neosartorya fischeri, Oryctolagus cuniculus, Oryza sativa, Penicillium cyclopium, Phlebotomus papatasi, Pseudomonas aeruginosa, Pseudomonasfluorescens, Pseudomonasfragi, Pseudomonas sp, Rattus norvegicus, Rhizomucor miehei, Rhizopus oryzae, Rhizopus stolonifer, Ricinus communis, Samia cynthia ricini, Schizosaccharomyces pombe, Serratia marcescens, Spermophilus tridecemlineatus, Staphylococcus simulans, Staphylococcus xylosus, Sulfolobus solfataricus, Sus scrofa, Thermomyces lanuginosus, Trichomonas vaginalis, Vibrio harveyi, Xenopus laevis, Yarrowia lipolytica, or a combination thereof.

45. The composition of claim 44, wherein the lipase comprises a themophilic lipase derived from Acinetobacter calcoaceticus, Acinetobacter sp., Bacillus sphaericus, Bacillus stearothermophilus, Bacillus thermocatenulatus, Bacillus thermoleovorans, Candida rugosa, Candida thermophila, GeoBacillus thermoleovorans Toshki, Pseudomonas fragi, Staphylococcus xylosus, Sulfolobus solfataricus, or a combination thereof.

46. The composition of claim 44, wherein the lipase comprises a psychrophilic lipase derived from Pseudomonas fluorescens.

47. The composition of claim 40, wherein the lipolytic enzyme comprises a lipoprotein lipase derived from Capra hircus, Danio rerio, Felis catus, Homo sapiens, Mesocricetus auratus, Mus musculus, Oncorhynchus mykiss, Pagrus major, Papio Anubis, Rattus norvegicus, Sparus aurata, Sus scrofa, Thunnus orientalis, or a combination thereof.

48. The composition of claim 40, wherein the lipolytic enzyme comprises an acylglycerol lipase derived from Bacillus sp., Danio rerio, Homo sapiens, Leishmania infantum, Mus musculus, Mycobacterium tuberculosis, Penicillium camembertii, Rattus norvegicus, Solanum tuberosum, or a combination thereof.

49. The composition of claim 40, wherein the lipolytic enzyme comprises a hormone sensitive lipase derived from Bos Taurus, Homo sapiens, Mus musculus, Rattus norvegicus, Spermophilus tridecemlineatus, Sus scrofa, Tetrahymena thermophila, or a combination thereof.

50. The composition of claim 40, wherein the lipolytic enzyme comprises a phospholipase A₁ derived from Arabidopsis, Aspergillus oryzae, Bos Taurus, Brassica rapa, Caenorhabditis elegans, Capsicum annuum, Danio rerio, Homo sapiens, Mus musculus, Nicotiana tabacum, Polistes annularis, Polybia paulista, Rattus norvegicus, Serratia sp., Vespula vulgaris, or a combination thereof.

51. The composition of claim 40, wherein the lipolytic enzyme comprises a phospholipase A₂ derived from Acanthaster planci, Adamsia carciniopado, Aedes aegypti, Aeropyrum pernix, Aipysurus eydouxii, Apis mellifera, Arabidopsis thaliana, Aspergillus nidulans, Austrelaps superbus, Bitis gabonica, Bos taurus, Bothriechis schlegelii, Bothropsjararacussu, BrachyDanio rerio, Bungarus caeruleus, Bungarus fasciatus, Canis familiaris, Cavia sp., Cerrophidion godmani, Chlamydomonas reinhardtii, Chrysophrys major, Crotalus viridis viridis, Daboia russellii, Danio rerio, Drosophila melanogaster, Echis carinatus, Echis ocellatus, Echis pyramidum leakeyi, Emericella nidulans, Equus caballus, Gallus gallus, Homo sapiens, Lapemis hardwickii, Laticauda semifasciata, Micrurus corallines, Mus musculus, Mytilus edulis, Naja kaouthia, Naja naja, Naja naja sputatrix, Nicotiana tabacum, Ophiophagus hannah, Ornithodoros parkeri, Oryctolagus cuniculus, Pagrus major, Patiria pectinifera, Polyandrocarpa misakiensis, Protobothrops mucrosquamatus, Rattus norvegicus, Sistrurus catenatus tergeminus, Trimeresurus borneensis, Trimeresurusflavoviridis, Trimeresurus gracilis, Trim eresurus gramineus, Trimeresurus okinavensis, Trimeresurus puniceus, Trimeresurus stejnegeri, Tuber borchii, Urticina crassicornis, Vipera russelli siamensis, Xenopus laevis, Xenopus tropicalis, or a combination thereof.

52. The composition of claim 51, wherein the phospholipase A₂ comprises a thermophilic phospholipase A₂ derived from Aeropyrum pernix.

53. The composition of claim 40, wherein the lipolytic enzyme comprises a phospholipase C derived from Aedes aegypti, Aplysia californica, Arabidopsis thaliana, Asterina miniata, Bacillus cereus, Bacillus thuringiensis, Bos taurus, Caenorhabditis elegans, Chaetopterus pergamentaceus, Chlamydomonas reinhardtii, Coturnix japonica, Danio rerio, Dictyostelium discoideum, Drosophila melanogaster, Gallus gallus, Homarus americanus, Homo sapiens, Loligo pealei, Lytechinus pictus, Meleagris gallopavo, Misgurnus mizolepis, Mus musculus, Nicotiana tabacum, Oryza sativa, Oryzias latipes, Petunia inflate, Pichia stipitis, Pisum sativum, Plasmodium falciparum, Rattus norvegicus, Strongylocentrotus purpuratus, Sus scrofa, Torenia fournieri, Toxoplasma gondii, Watasenia scintillans, Xenopus laevis, Zea mays, or a combination thereof.

54. The composition of claim 53, wherein the phospholipase C comprises a thermophilic phospholipase C derived from Bacillus cereus.

55. The composition of claim 40, wherein the lipolytic enzyme comprises a phospholipase D derived from Aedes aegypti, Arabidopsis thaliana, Arachis hypogaea, Bos taurus, Brassica oleracea, Caenorhabditis elegans, Cricetulus griseus, Cucumis melo var. inodorus, Cucumis sativus, Dictyostelium discoideum, Drosophila melanogaster, Emericella nidulans, Fragaria ananassa, Gossypium hirsutum, Homo sapiens, Lolium temulentum, Lycopersicon esculentum, Mus musculus, Oryza sativa, Papaver somniferum, Paralichthys olivaceus, Pichia stipitis, Pimpinella brachycarpa, Rattus norvegicus, Ricinus communis, Streptoverticillium cinnamoneum, Vigna unguiculata, Vitis vinifera, Zea mays, or a combination thereof.

56. The composition of claim 40, wherein the lipolytic enzyme comprises a phosphoinositide phospholipase C derived from Arabidopsis thaliana, Aspergillus clavatus, Aspergillus fumigatus, Brassica napus, Homo sapiens, Leishmania infantum, Mus musculus, Neosartoryafischeri, Physcomitrella patens, Pichia stipitis, Rattus norvegicus, Toxoplasma gondii, Trypanosoma brucei, Vigna unguiculata, Xenopus tropicalis, Zea mays, or a combination thereof.

57. The composition of claim 40, wherein the lipolytic enzyme comprises a phosphatidate phosphatase derived from Saccharomyces cerevisiae, or a combination thereof.

58. The composition of claim 40, wherein the lipolytic enzyme comprises a lysophospholipase derived from Aedes aegypti, Argas monolakensis, Aspergillus clavatus, Aspergillus fumigatus, Bos Taurus, Cavia porcellus, Clonorchis sinensis, Danio rerio, Dictyostelium discoideum, Emericella nidulans, Giardia lamblia, Homo sapiens, Monodelphis domestica, Mus musculus, Neosartoryafischeri, Pichia jadinii, Pichia stipitis, Rattus norvegicus, Schistosomajaponicum, Schizosaccharomyces pombe, Sclerotinia sclerotiorum, Xenopus tropicalis, or a combination thereof.

59. The composition of claim 40, wherein the lipolytic enzyme comprises a sterol esterase derived from Candida rugosa, Homo sapiens, Melanocarpus albomyces, Rattus norvegicus, or a combination thereof.

60. The composition of claim 40, wherein the lipolytic enzyme comprises a galactolipase derived from Homo sapiens, Solanum tuberosum, Vigna unguiculata, or a combination thereof.

61. The composition of claim 40, wherein the lipolytic enzyme comprises a sphingomyelin phosphodiesterase derived from Bacillus cereus, Homo sapiens, Pseudomonas sp., or a combination thereof.

62. The composition of claim 40, wherein the lipolytic enzyme comprises a ceramidase derived from Homo sapiens, Pseudomonas, or a combination thereof.

63. The composition of claim 40, wherein the lipolytic enzyme comprises a retinyl palmitate esterase derived from Bos Taurus.

64. The composition of claim 40, wherein the lipolytic enzyme comprises a cutinase derived from Fusarium solani pisi, Monilinia fructicola, Pseudomonas putida, or a combination thereof.

65. The composition of claim 1, wherein the active enzyme comprises a mesophilic enzyme, a psychrophilic enzyme, a thermophilic enzyme, a halophilic enzyme, or a combination thereof.

66. The composition of claim 1, wherein the coating comprises a stimulator of enzyme activity.

67. The composition of claim 1, wherein the active enzyme comprises an immobilization carrier.

68. The composition of claim 42, wherein the immobilization carrier comprises a reverse micelle, zeolite, Celite Hyflo Supercel, a resin, diatomaceous earth, a polyurethane foam particle, macroporous polypropylene Accurel® EP 100, a macroporous anionic resin bead, polypropylene membrane, acrylic membrane, nylon membrane, cellulose ester membrane, polyvinylidene difuoride membrane, filter paper, teflon membrane, ceramic membrane, a macroporous packing particulate, polyamide, cellulose hollow fibre, a polypropylene membrane pretreated with a blocked copolymer, an immunoglobin, agarose, a gel, or a combination thereof.

69. The composition of claim 1, wherein the active enzyme comprises a purified active enzyme.

70. The composition of claim 1, wherein the active enzyme comprises a cell-based particulate material.

71. The composition of claim 70, wherein the cell-based particulate material comprises a microorganism-based particulate material.

72. The composition of claim 70, wherein the cell-based particulate material is a whole cell particulate material.

73. The composition of claim 70, wherein the cell-based particulate material is a cell fragment particulate material.

74. The composition of claim 1, wherein the active enzyme is prepared in a material that is attenuated.

75. The composition of claim 1, wherein the active enzyme is prepared in a material that is sterilized.

76. The composition of claim 1, wherein the active enzyme comprises a petroleum lipolytic enzyme.

77. The composition of claim 1, wherein the active enzyme comprises about 0.1% to about 80% of the coating by weight or volume.

78. The composition of claim 1, wherein the active enzyme comprises a particulate material.

79. The composition of claim 78, wherein the average wet molecular weight or dry molecular weight of a primary particle of the particulate material is about 50 kDa to about 1.5×10¹⁴ kDa.

80. The composition of claim 69, wherein the average active enzyme content per primary particle of the particulate material is about 0.0000001% to about 100%.

81. The composition of claim 1, wherein the coating is about 5 um to about 5000 um thick upon a surface.

82. The composition of claim 1, wherein the coating comprises a paint.

83. The composition of claim 1, wherein the coating comprises a clear coating.

84. The composition of claim 93, wherein the clear coating comprises a lacquer, a varnish, a shellac, a stain, a water repellent coating, or a combination thereof.

85. The composition of claim 1, wherein the coating comprises a multicoat system.

86. The composition of claim 85, wherein the multicoat system comprises 2 to 10 layers.

87. The composition of claim 85, wherein one layer of the multicoat system comprises the active enzyme.

88. The composition of claim 85, wherein a plurality of layers of the multicoat system comprise the active enzyme.

89. The composition of claim 88, wherein at least one layer of said plurality of layers comprises a different preparation of the active enzyme than at least a second layer of said plurality of layers that comprises the active enzyme.

90. The composition of claim 85, wherein each layer of the multicoat system is coating is about 5 um to about 5000 um thick upon a surface.

91. The composition of claim 85, wherein the multicoat system comprises a sealer, a water repellent, a primer, an undercoat, a topcoat, or a combination thereof.

92. The composition of claim 85, wherein the multicoat system comprises a topcoat.

93. The composition of claim 92, wherein the topcoat comprises the active enzyme.

94. The composition of claim 1, wherein the coating is a coating that is capable of film formation.

95. The composition of claim 94, wherein film formation occurs between about −101C to about 40° C.

96. The composition of claim 94, wherein film formation occurs at baking conditions.

97. The composition of claim 94, wherein the coating comprises a volatile component and a non-volatile component.

98. The composition of claim 97, wherein the coating undergoes film formation by loss of part of the volatile component.

99. The composition of claim 94, wherein film formation occurs by crosslinking of a binder.

100. The composition of claim 99, wherein film formation occurs by crosslinking of a plurality of binders.

101. The composition of claim 94, wherein film formation occurs by irradiating the coating.

102. The composition of claim 94, wherein the coating produces a self-cleaning film.

103. The composition of claim 94, wherein the coating produces a temporary film.

104. The composition of claim 1, wherein the coating is a non-film forming coating.

105. The composition of claim 1, wherein the coating comprises architectural coating.

106. The composition of claim 105, wherein the coating comprises an architectural wood coating, an architectural masonry coating, an architectural artist's coating, an architectural plastic coating, an architectural metal coating, or a combination thereof.

107. The composition of claim 105, wherein the coating has a pot life of at least 12 months at about −10° C. to about 40° C.

108. The composition of claim 105, wherein the coating undergoes film formation between about −101C to about 40° C.

109. The composition of claim 1, wherein the coating comprises an automotive coating, a can coating, a sealant coating, or a combination thereof.

110. The composition of claim 109, wherein the coating undergoes film formation at baking conditions.

111. The composition of claim 1, wherein the coating comprises a chemical agent resistant coating, a camouflage coating, a pipeline coating, a traffic marker coating, an aircraft coating, a nuclear power plant coating, or a combination thereof.

112. The composition of claim 1, wherein the coating comprises a plastic coating.

113. The composition of claim 1, wherein the coating comprises a water-borne coating.

114. The composition of claim 113, wherein the water-borne coating is a latex coating.

115. The composition of claim 113, wherein the water-borne coating has a density of about 1.20 kg/L to about 1.50 kg/L.

116. The composition of claim 1, wherein the coating comprises a solvent-borne coating.

117. The composition of claim 116, wherein the solvent-borne coating has a density of about 0.90 kg/L to about 1.2 kg/L.

118. The composition of claim 1, wherein the coating has a low-shear viscosity of about 100 P to about 3000 P.

119. The composition of claim 1, wherein the coating comprises a binder, a liquid component, a colorant, an additive, or a combination thereof.

120. The composition of claim 119, wherein the coating comprises a binder.

121. The composition of claim 120, wherein the binder comprises a thermoplastic binder, a thermosetting binder, or a combination thereof.

122. The composition of claim 121, wherein the coating comprises a thermoplastic binder.

123. The composition of claim 122, wherein the coating is a coating capable of producing a film by thermoplastic film formation.

124. The composition of claim 121, wherein the coating comprises a thermosetting binder.

125. The composition of claim 124, wherein the coating is a coating capable of producing a film by thermosetting film formation.

126. The composition of claim 119, wherein the coating comprises a liquid component.

127. The composition of claim 126, wherein the liquid component comprises a solvent, a thinner, a diluent, a plasticizer, or a combination thereof.

128. The composition of claim 126, wherein the liquid component comprises a liquid organic compound, an inorganic compound, water, or a combination thereof.

129. The composition of claim 1, wherein the coating comprises a colorant.

130. The composition of claim 129, wherein the colorant comprises a pigment, a dye, or a combination thereof.

131. The composition of claim 131, wherein the coating comprises a metal surface coating.

132. The composition of claim 133, wherein the coating comprises an additive.

133. The composition of claim 132, wherein the additive comprises 0.000001% to 20.0% by weight, of the coating.

134. The composition of claim 133, wherein said additive comprises an accelerator, an adhesion promoter, an antifoamer, anti-insect additive, an antioxidant, an antiskinning agent, a buffer, a catalyst, a coalescing agent, a corrosion inhibitor, a defoamer, a dehydrator, a dispersant, a drier, electrical additive, an emulsifier, a filler, a flame/fire retardant, a flatting agent, a flow control agent, a gloss aid, a leveling agent, a marproofing agent, a preservative, a silicone additive, a slip agent, a surfactant, a light stabilizer, a rheological control agent, a wetting additive, a cryopreservative, a xeroprotectant, or a combination thereof.

135. The composition of claim 134, wherein the additive comprises a preservative.

136. The composition of claim 135, wherein the preservative comprises an in-can preservative, an in-film preservative, or a combination thereof.

137. The composition of claim 135, wherein the preservative comprises a biocide, a biostatic, or a combination thereof.

138. The composition of claim 137, wherein the biocide comprises a bactericide, a fungicide, an algaecide, or a combination thereof.

139. The composition of claim 135, wherein the preservative comprises 1-(3-chloroallyl)-3,5,7-triaza-1-azoniaadamantane chloride; 1,2-benzisothiazoline-3-one; 1,2-dibromo-2,4-dicyanobutane; 1,3-bis(hydroxymethyl)-5,5-dimethylhydantoin; 1-methyl-3,5,7-triaza-1-azonia-adamantane chloride; 2-bromo-2-nitropropane-1,3-diol; 2-(4-thiazolyl)benzimidazole; 2-(hydroxymethyl)-amino-2-methyl-1-propanol; 2(hydroxymethyl)-aminoethanol; 2,2-dibromo-3-nitrilopropionamide; 2,4,5,6-tetrachloro-isophthalonitrile; 2-mercaptobenzo-thiazole; 2-methyl-4-isothiazolin-3-one; 2-n-octyl-4-isothiazoline-3-one; 3-iodo-2-propynl N-butyl carbamate; 4,5-dichloro-2-N-octyl-3(2H)-isothiazolone; 4,4-dimethyloxazolidine; 5-chloro-2-methyl-4-isothiazolin-3-one; 5-hydroxy-methyl-1-aza-3,7-dioxabicylco(3.3.0.)octane; 6-acetoxy-2,4-dimethyl-1,3-dioxane; 7-ethyl bicyclooxazolidine; a combination of 1,2-benzisothiazoline-3-one and hexahydro-1,3,5-tris(2-hydroxyethyl)-s-triazine; a combination of 1,2-benzisothiazoline-3-one and zinc pyrithione; a combination of 2-(thiocyanomethyl-thio)benzothiozole and methylene bis(thiocyanate); a combination of 4-(2-nitrobutyl)-morpholine and 4,4′-(2-ethylnitrotrimethylene)dimorpholine; a combination of 4,4-dimethyl-oxazolidine and 3,4,4-trimethyloxazolidine; a combination of 5-chloro-2-methyl-4-isothiazolin-3-one and 2-methyl-4-isothiazolin-3-one; a combination of carbendazim and 3-iodo-2-propynl N-butyl carbamate; a combination of carbendazim, 3-iodo-2-propynl N-butyl carbamate and diuron; a combination of chlorothalonil and 3-iodo-2-propynl N-butyl carbamate; a combination of chlorothalonil and a triazine compound; a combination of tributyltin benzoate and alkylamine hydrochlorides; a combination of zinc-dimethyldithiocarbamate and zinc 2-mercaptobenzothiazole; a copper soap; a metal soap; a mercury soap; a mixture of bicyclic oxazolidines; a tin soap; an alkylamine hydrochloride; an amine reaction product; barium metaborate; butyl parahydroxybenzoate; carbendazim; copper(II) 8-quinolinolate; diiodomethyl-p-tolysulfone; dithio-2,2-bis(benzmethylamide); diuron; ethyl parahydroxybenzoate; glutaraldehyde; hexahydro-1,3,5-triethyl-s-triazine; hexahydro-1,3,5-tris(2-hydroxyethyl)-s-triazine; hydroxymethyl-5,5-dimethylhydantoin; methyl parahydroxybenzoate; N-butyl-1,2-benzisothiazolin-3-one; N-(trichloromethylthio)phthalimide; N-cyclopropyl-N-(1-dimethylethyl)-6-(methylthio)-1,3,5-triazine-2,4-diamine; N-trichloromethyl-thio-4-cyclohexene-1,2-dicarboximide; p-chloro-m-cresol; phenoxyethanol; phenylmercuric acetate; poly(hexamethylene biguanide) hydrochloride; potassium dimethyldithiocarbamate; potassium N-hydroxy-methyl-N-methyl-dithiocarbamate; propyl parahydroxybenzoate; sodium 2-pyridinethiol-1-oxide; tetra-hydro-3,5-di-methyl-2H-1,3,5-thiadiazine-2-thione; tributyltin benzoate; tributyltin oxide; tributyltin salicylate; zinc pyrithione; sodium pyrithione; copper pyrithione; zinc oxide; a zinc soap; or a combination thereof.

140. The composition of claim 1, wherein the coating is a multi-pack coating.

141. The composition of claim 140, wherein the multi-pack coating is stored in a two to five containers prior to application to a surface.

142. The composition of claim 140, wherein about 0.000001% to about 100% of the active enzyme is stored in a container of the multi-pack coating, and at least one coating component is stored in another container of the multi-pack coating.

143. The composition of claim 1, wherein the coating is a coating capable of being applied to a surface by a spray applicator.

144. The composition of claim 1, wherein the active enzyme is microencapsulated.

145. The composition of claim 1, wherein the coating comprises a pH indicator.

146. The composition of claim 1, wherein the esterase comprises a phosphoric triester hydrolase.

147. The composition of claim 1, wherein the esterase comprises a plurality of phosphoric triester hydrolases.

148. The composition of claim 1, wherein the active enzyme comprises an esterase.

149. The composition of claim 1, wherein the active enzyme comprises a plurality of esterases.

150. The composition of claim 1, wherein the active enzyme comprises a peptidase.

151. The composition of claim 1, wherein the active enzyme comprises a plurality of peptidases.

152. The composition of claim 150, wherein the peptidase comprises a chymotrypsin, trypsin, or a combination thereof.

153. The composition of claim 1, wherein the esterase comprises a sulfuric ester hydrolase.

154. The composition of claim 153, wherein the esterase comprises a plurality of sulfuric ester hydrolases.

155. A coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises an esterase.

156. An architectural coating composition, comprising an architectural coating that comprises an active enzyme, wherein the active enzyme comprises an antimicrobial enzyme, a peptidase, an esterase, a petroleum lipolytic enzyme, or a combination thereof.

157. An architectural wood coating composition, comprising an architectural wood coating that comprises an active enzyme, wherein the active enzyme comprises an antimicrobial enzyme, a peptidase, an esterase, a petroleum lipolytic enzyme, or a combination thereof.

158. An architectural masonry coating composition, comprising an architectural masonry coating that comprises an active enzyme, wherein the active enzyme comprises an antimicrobial enzyme, a peptidase, an esterase, a petroleum lipolytic enzyme, or a combination thereof.

159. An architectural artist's coating composition, comprising an architectural artist's coating that comprises an active enzyme, wherein the active enzyme comprises an antimicrobial enzyme, a peptidase, an esterase, a petroleum lipolytic enzyme, or a combination thereof.

160. An architectural plastic coating composition, comprising an architectural plastic coating that comprises an active enzyme, wherein the active enzyme comprises an antimicrobial enzyme, a peptidase, an esterase, a petroleum lipolytic enzyme, or a combination thereof.

161. An architectural metal coating composition, comprising an architectural metal coating that comprises an active enzyme, wherein the active enzyme comprises an antimicrobial enzyme, a peptidase, an esterase, a petroleum lipolytic enzyme, or a combination thereof.

162. A clear coating composition, comprising a clear coating that comprises an active enzyme, wherein the active enzyme comprises an antimicrobial enzyme, a peptidase, an esterase, a petroleum lipolytic enzyme, or a combination thereof.

163. An automotive coating composition, comprising an automotive coating that comprises an active enzyme, wherein the active enzyme comprises an antimicrobial enzyme, a peptidase, an esterase, a petroleum lipolytic enzyme, or a combination thereof.

164. A can coating composition, comprising a can coating that comprises an active enzyme, wherein the active enzyme comprises an antimicrobial enzyme, a peptidase, an esterase, a petroleum lipolytic enzyme, or a combination thereof.

165. A sealant coating composition, comprising a sealant coating that comprises an active enzyme, wherein the active enzyme comprises an antimicrobial enzyme, a peptidase, an esterase, a petroleum lipolytic enzyme, or a combination thereof.

166. A chemical agent resistant coating coating composition, comprising a chemical agent resistant coating coating that comprises an active enzyme, wherein the active enzyme comprises an antimicrobial enzyme, a peptidase, an esterase, a petroleum lipolytic enzyme, or a combination thereof.

167. A camouflage coating composition, comprising a camouflage coating that comprises an active enzyme, wherein the active enzyme comprises an antimicrobial enzyme, a peptidase, an esterase, a petroleum lipolytic enzyme, or a combination thereof.

168. A specification coating composition, comprising a specification coating that comprises an active enzyme, wherein the active enzyme comprises an antimicrobial enzyme, a peptidase, an esterase, a petroleum lipolytic enzyme, or a combination thereof.

169. A pipeline coating composition, comprising a pipeline coating that comprises an active enzyme, wherein the active enzyme comprises an antimicrobial enzyme, a peptidase, an esterase, a petroleum lipolytic enzyme, or a combination thereof.

170. A traffic marker coating composition, comprising a traffic marker coating that comprises an active enzyme, wherein the active enzyme comprises an antimicrobial enzyme, a peptidase, an esterase, a petroleum lipolytic enzyme, or a combination thereof.

171. An aircraft coating composition, comprising an aircraft coating that comprises an active enzyme, wherein the active enzyme comprises an antimicrobial enzyme, a peptidase, an esterase, a petroleum lipolytic enzyme, or a combination thereof.

172. A nuclear power plant coating composition, comprising a nuclear power plant coating that comprises an active enzyme, wherein the active enzyme comprises an antimicrobial enzyme, a peptidase, an esterase, a petroleum lipolytic enzyme, or a combination thereof.

173. A water-borne coating composition, comprising a water-borne coating that comprises an active enzyme, wherein the active enzyme comprises an antimicrobial enzyme, a peptidase, an esterase, a petroleum lipolytic enzyme, or a combination thereof.

174. A solvent-borne coating composition, comprising a solvent-borne coating that comprises an active enzyme, wherein the active enzyme comprises an antimicrobial enzyme, a peptidase, an esterase, a petroleum lipolytic enzyme, or a combination thereof.

175. A powder coating composition, comprising a powder coating and an active enzyme, wherein the active enzyme comprises an antimicrobial enzyme, a peptidase, an esterase, a petroleum lipolytic enzyme, or a combination thereof.

176. A multi-pack coating composition, comprising a plurality of containers, wherein at least one container comprises an active enzyme, and wherein the coating comprises an architectural wood coating, an architectural masonry coating, an architectural artist coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises an antimicrobial enzyme, a peptidase, an esterase, a petroleum lipolytic enzyme, or a combination thereof.

177. An elastomer composition, comprising an elastomer and an active enzyme, wherein the active enzyme comprises an antimicrobial enzyme, a peptidase, an esterase, a petroleum lipolytic enzyme, or a combination thereof.

178. A filler composition, comprising a filler and an active enzyme, wherein the active enzyme comprises an antimicrobial enzyme, a peptidase, an esterase, a petroleum lipolytic enzyme, or a combination thereof.

179. An adhesive composition, comprising an adhesive and an active enzyme, wherein the active enzyme comprises an antimicrobial enzyme, a peptidase, an esterase, a petroleum lipolytic enzyme, or a combination thereof.

180. A sealant composition, comprising a sealant and an active enzyme, wherein the active enzyme comprises an antimicrobial enzyme, a peptidase, an esterase, a petroleum lipolytic enzyme, or a combination thereof.

181. A textile finish composition, comprising a textile finish and an active enzyme, wherein the active enzyme comprises an antimicrobial enzyme, a peptidase, an esterase, a petroleum lipolytic enzyme, or a combination thereof.

182. A wax, comprising a wax and an active enzyme, wherein the active enzyme comprises an antimicrobial enzyme, a peptidase, an esterase, a petroleum lipolytic enzyme, or a combination thereof.

183. A method of inhibiting microbical grown on a surface, comprising the steps of: contacting a surface contaminated with a microorganism with a coating comprising an active enzyme, wherein the active enzyme comprises an antimicrobical enzyme.

184. A kit having component parts capable of being assembled comprising a container comprising an active enzyme, and a container comprising at least one component of a surface treatment, wherein the active enzyme comprises an antimicrobial enzyme, a peptidase, an esterase, a petroleum lipolytic enzyme, or a combination thereof.

185. An article of manufacture having enzyme activity, comprising an manufactured object, wherein at least one surface of the object comprises a surface treatment, wherein the surface treatment comprises an active enzyme comprising an antimicrobial enzyme, a peptidase, an esterase, a petroleum lipolytic enzyme, or a combination thereof.

186. A coating composition, comprising a coating that comprises an active enzyme, wherein the coating comprises an architectural coating, an automotive coating, a can coating, a sealant coating, a chemical agent resistant coating, a camouflage coating, a pipeline coating, a traffic marker coating, an aircraft coating, a nuclear power plant coating, or a combination thereof, and wherein the active enzyme comprises an antimicrobial enzyme.

187. A coating composition of claim 271, wherein the active enzyme comprises a peptidase, an esterase, a petroleum lipolytic enzyme, or a combination thereof.
